(19) United States
(12) Patent Application Publication
(10) Pub. No.: US 2026/0150499 A1
(43) Pub. Date: May 28, 2026

(54) Organic Compounds and Electroluminescent Device Comprising the Same

(71) Applicant: P&H TECH Co., Ltd, Yongin-si (KR)

(72) Inventors: Gwan-hee PARK, Yongin-si (KR); Yeonju CHO, Yongin-si (KR); Yukyung LEE, Yongin-si (KR)

(73) Assignee: P&H TECH Co., Ltd, Yongin-si (KR)

(21) Appl. No.: 19/396,903

(22) Filed: Nov. 21, 2025

(30) Foreign Application Priority Data
Nov. 27, 2024 (KR) 10-2024-0171649
Nov. 6, 2025 (KR) 10-2025-0165826

(51) Int. Cl. H10K 50/858; C07D 235/02; C07D 263/52; C07D 413/14; C07D 417/14; C09K 11/02

(52) U.S. Cl. CPC H10K 50/858; C07D 235/02; C07D 263/52; C07D 413/14; C07D 417/14; C09K 11/025

(57) ABSTRACT

Disclosed are a compound represented by the following [Chemical Formula I], which can be employed in a light efficiency improving layer provided in an organic light-emitting device to implement a highly efficient and long-lifetime organic light-emitting device with improved luminous efficiency, color purity, life characteristics, and the like as well as low-voltage driving characteristics, and an organic light-emitting device including the same.

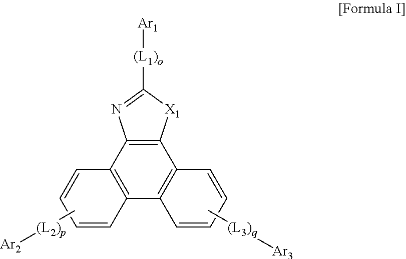

### CROSS-REFERENCE TO RELATED APPLICATIONS

This application claims priority to and the benefit of Korean Patent Application No. 10-2024-0171649 filed in the Korean Intellectual Property Office on Nov. 27, 2024, the entire contents of which are incorporated herein by reference.

### TECHNICAL FIELD

The present invention relates to a novel organic compound that is employed as a material for a light efficiency improving layer (capping layer) provided in an organic light-emitting device, and to an organic light-emitting device in which device light-emitting properties such as low voltage driving, light-emitting efficiency, color purity, and service life are remarkably improved by employing the same in the light efficiency improving layer.

### BACKGROUND ART

The organic light emitting device may be formed even on a transparent substrate, and may be driven at a low voltage of 10 V or less compared to a plasma display panel or an inorganic electroluminescence (EL) display. In addition, the device consumes relatively little power and has good color representation. The device may display three colors of green, blue, and red, and thus has recently become a subject of intense interest as a next-generation display device.

However, in order for such an organic light emitting device to exhibit the aforementioned characteristics, the materials constituting an organic layer in the device, such as hole injecting materials, hole transport materials, light emitting materials, electron transport materials, and electron injecting materials, are prerequisites for the support by stable and efficient materials. However, the development of a stable and efficient organic layer material for an organic light emitting device has not yet been sufficiently made.

Thus, further improvements in terms of efficiency and life characteristics are required for good stability, high efficiency, long lifetime, and large size of organic light emitting devices. Particularly, there is a strong need to develop materials constituting each organic layer of organic light emitting devices.

In addition, recently, research aimed at improving the characteristics of organic light emitting devices by changes in the performance of each organic layer material, as well as a technique for improving the color purity and enhancing the luminous efficiency by optimizing the optical thickness between an anode and a cathode are considered as one of the crucial factors for improving the device performance. As an example of this method, an increase in light efficiency and excellent color purity are achieved by using a capping layer on an electrode.

The efficiency of the organic light-emitting device may be divided into internal light-emitting efficiency and external light-emitting efficiency, the internal light-emitting efficiency is related to the efficiency of exciton production and light conversion in various organic layers interposed between a first electrode and a second electrode, such as a hole transport layer, a light-emitting layer, and an electron transport layer, the external light-emitting efficiency is the efficiency at which light generated in the organic layer is extracted to the outside of the organic light-emitting device, and in order to increase such a light extraction efficiency, a light efficiency improving layer (capping layer) with an optimally adjusted refractive index is applied.

However, there is an urgent need for designing and developing an optimized light efficiency improving layer such that it is possible to implement a highly efficient device without impairing process efficiency such as deposition and other light-emitting characteristics including the service life characteristics and the like of a device.

### SUMMARY OF THE INVENTION

The present disclosure has been made in an effort to provide an organic compound which may be employed in a light efficiency improving layer (capping layer) provided in an organic light-emitting device to implement excellent luminescent properties such as low-voltage driving of the device and improved luminous efficiency, and an organic light-emitting device including the same.

An embodiment of the present disclosure provides an organic compound for a light efficiency improving layer (capping layer) represented by the following [Formula I].

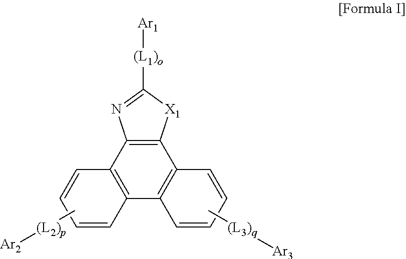

The characteristic structure of [Formula I] above and the definitions of the specific compound implemented by the same, X₁, L₁ to L₃, and Ar₁ to Ar₃ will be described later.

Another exemplary embodiment provides an organic light-emitting device including a first electrode, a second electrode, and an organic layer having one or more layers disposed between the first electrode and the second electrode, in which the organic light-emitting device further includes a light efficiency improving layer (capping layer) formed on at least one side opposite to the organic layer at the top or bottom part of the first electrode and the second electrode and the light efficiency improving layer includes the organic compound represented by [Formula I].

The organic compound according to the present invention can improve the light efficiency extracted to the outside of an organic light-emitting device, and thus, can be usefully used as a material for a light efficiency improving layer provided in an organic light-emitting device. Therefore, it is possible to implement a highly efficient and long-lifetime organic light-emitting device with improved luminous efficiency, color purity, life characteristics, and the like as well as low-voltage driving characteristics by employing the compound according to the present invention in the light efficiency improving layer, and thus, the compound can be usefully used in various lighting and display devices.

### BRIEF DESCRIPTION OF THE DRAWINGS

FIG. 1 is a schematic cross-sectional view illustrating the concept and configuration of an organic light-emitting device according to the present invention.

FIG. 2 is a schematic cross-sectional view illustrating the configuration of an organic light-emitting device according to an embodiment of the present invention.

FIG. 3 is a schematic cross-sectional view illustrating the configuration of an organic light-emitting device according to an embodiment of the present invention.

### DETAILED DESCRIPTION

Hereinafter, the present invention will be described in more detail.

The compound for a light efficiency improving layer according to the present invention is characterized by a compound in which three moieties having characteristic structures are introduced into a skeleton represented by [Formula I] below.

Due to these structural features, the organic compound represented by [Formula I] according to the present invention may be used as a material for a light efficiency improving layer (capping layer) provided in an organic light-emitting device to implement a highly efficient organic light-emitting device with improved luminous efficiency, color purity, and life characteristics, and the like as well as low-voltage driving characteristics.

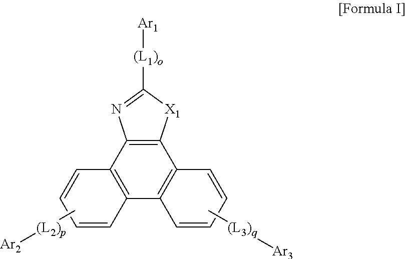

In [Formula I] above,
X₁ is any one selected from O, S, SO (sulfur monoxide), SO₂ (sulfur dioxide), NR₁, CR₂R₃, SiR₄R₅, and GeR₆R₇.
R₁ to R₇ are the same as or different from each other, and are each independently selected from hydrogen, deuterium, a substituted or unsubstituted alkyl group having 1 to 20 carbon atoms, a substituted or unsubstituted alkoxy group having 1 to 20 carbon atoms, a substituted or unsubstituted cycloalkyl group having 3 to 20 carbon atoms, a substituted or unsubstituted heterocycloalkyl group having 3 to 30 carbon atoms, a substituted or unsubstituted aryl group having 6 to 30 carbon atoms, a substituted or unsubstituted heteroaryl group having 2 to 50 carbon atoms, a substituted or unsubstituted silyl group, a substituted or unsubstituted germanium group, a cyano group, and a halogen group.
L₁ to L₃ are a divalent linker, and are a direct bond or selected from a substituted or unsubstituted arylene group having 6 to 30 carbon atoms, and a substituted or unsubstituted heteroarylene group having 2 to 50 carbon atoms, o, p, and q are an integer from 0 to 2, and when o, p, and q are 2, a plurality of L₁ to L₃ are each the same as or different from each other.
Ar₁ to Ar₃ are the same as or different from each other, and are each independently a cyano group (CN), or any one selected from a substituted or unsubstituted aryl group having 6 to 30 carbon atoms and a substituted or unsubstituted heteroaryl group having 2 to 50 carbon atoms.

According to an embodiment of the present invention, L₁ to L₃ are a divalent linking group, and may be a direct bond or a substituted or unsubstituted arylene having 6 to 30 carbon atoms, and according to a preferred embodiment, may be a direct bond or a substituted or unsubstituted phenylene group.

According to an embodiment of the present invention, Ar₁ to Ar₃ are the same as or different from each other, and may be each independently a substituted or unsubstituted aryl group having 6 to 30 carbon atoms, and according to a preferred embodiment, Ar₁ to Ar₃ may be each a substituted or unsubstituted naphthyl group.

According to an embodiment of the present invention, at least one or more of Ar₁ to Ar₃ may be the following [Structural Formula 1] or any one selected from the following [Structural Formula 2].

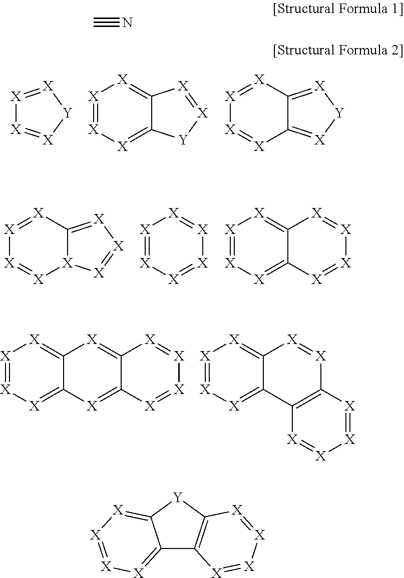

In each structural formula in [Structural Formula 2] above,
X's are the same as or different from each other, and are each independently N or CR₁₁, and Y is selected from O, S, NR₁₂, CR₁₃R₁₄, SiR₁₅R₁₆, and GeR₁₇R₁₈.

R₁₁ to R₁₈ are the same as or different from each other, and are each independently selected from hydrogen, deuterium, a cyano group, a halogen group, a substituted or unsubstituted alkyl group having 1 to 20 carbon atoms, a substituted or unsubstituted alkenyl group having 2 to 20 carbon atoms, an alkynyl group having 2 to 20 carbon atoms, a substituted or unsubstituted alkoxy group having 1 to 20 carbon atoms, a substituted or unsubstituted cycloalkyl group having 3 to 20 carbon atoms, a substituted or unsubstituted heterocycloalkyl group having 3 to 30 carbon atoms, a substituted or unsubstituted aryl group having 6 to 30 carbon atoms, a substituted or unsubstituted heteroaryl group having 2 to 50 carbon atoms, a substituted or unsubstituted amine group having 1 to 50 carbon atoms, a substituted or unsubstituted silyl group, and a substituted or unsubstituted germanium group.

The plurality of R₁₁'s may be linked to each other to form an alicyclic or aromatic monocyclic or polycyclic ring.

R₁₃ and R₁₄, R₁₅ and R₁₆, and R₁₇ and R₁₈ may be each linked to each other to form an alicyclic or aromatic monocyclic or polycyclic ring.

The carbon atoms of the formed alicyclic or aromatic monocyclic or polycyclic ring can be replaced by heteroatoms selected from O, S, N, P, Si, and Ge.

Any one selected from R₁₁ to R₁₈ in each structural formula is bonded to L₁ to L₃ in [Formula I] above.

According to an embodiment of the present invention, [Structural Formula 2] above may be more specifically represented by the following [Structural Formula 2-1], and accordingly, A1 to A4 are the same or different from each other, and may be each independently any one selected from the following [Structural Formula 2-1].

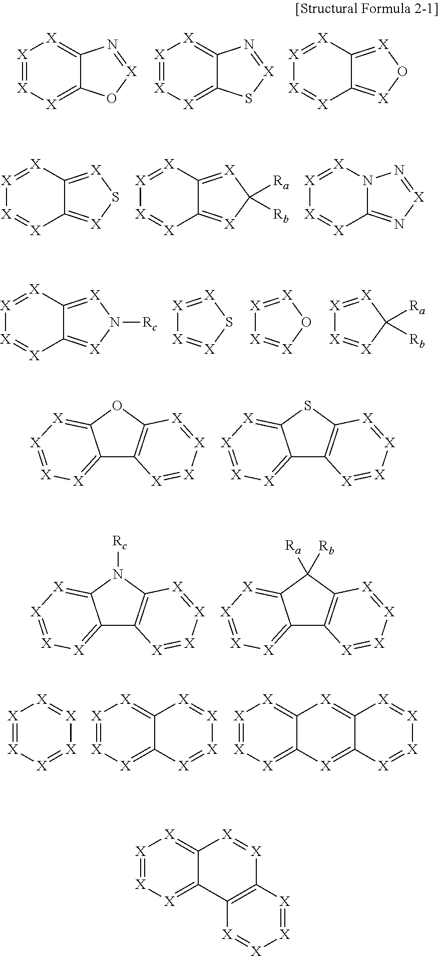

In each structural formula in [Structural Formula 2-1] above,
X's are the same as or different from each other, and are each independently N or CR₁₁, and Y is selected from O, S, NR₁₂, CR₁₃R₁₄, SiR₁₅R₁₆, and GeR₁₇R₁₈.
Rₐ to R_{c} and R₁₁ to R₁₈ are the same as or different from each other, and are each independently selected from hydrogen, deuterium, a cyano group, a halogen group, a substituted or unsubstituted alkyl group having 1 to 20 carbon atoms, a substituted or unsubstituted alkenyl group having 2 to 20 carbon atoms, an alkynyl group having 2 to 20 carbon atoms, a substituted or unsubstituted alkoxy group having 1 to 20 carbon atoms, a substituted or unsubstituted cycloalkyl group having 3 to 20 carbon atoms, a substituted or unsubstituted heterocycloalkyl group having 3 to 30 carbon atoms, a substituted or unsubstituted aryl group having 6 to 30 carbon atoms, a substituted or unsubstituted heteroaryl group having 2 to 50 carbon atoms, a substituted or unsubstituted amine group having 1 to 50 carbon atoms, a substituted or unsubstituted silyl group, and a substituted or unsubstituted germanium group.

The plurality of R₁₁'s may be linked to each other to form an alicyclic or aromatic monocyclic or polycyclic ring.

Further, Rₐ and R_{b}, R₁₃ and R₁₄, R₁₅ and R₁₆, and R₁₇ and R₁₈ may be each linked to each other to form an alicyclic or aromatic monocyclic or polycyclic ring.

In addition, the carbon atoms of the additionally formed alicyclic or aromatic monocyclic or polycyclic ring can be replaced by heteroatoms selected from O, S, N, P, Si, and Ge.

Any one selected from Rₐ to R_{c} and R₁₁ to R₁₈ in each structural formula is bonded to L₁ to L₃ in [Formula I] above.

According to an embodiment of the present invention, at least one or more of Ar₁ to Ar₃ may be the following [Structural Formula 1] or [Structural Formula 2-2].

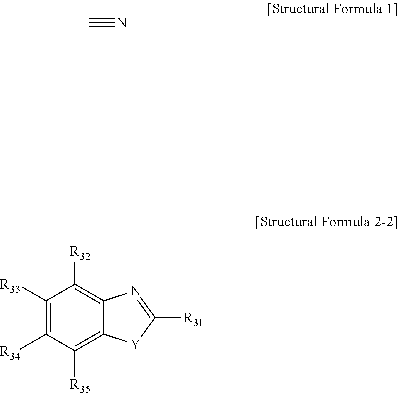

In [Structural Formula 2-2] above,
Y is selected from O, S, SO (sulfur monoxide), SO₂ (sulfur dioxide), and NR₂₁, and R₂₁ is selected from hydrogen, deuterium, a substituted or unsubstituted alkyl group having 1 to 20 carbon atoms, a substituted or unsubstituted alkoxy group having 1 to 20 carbon atoms, a substituted or unsubstituted cycloalkyl group having 3 to 20 carbon atoms, a substituted or unsubstituted heterocycloalkyl group having 3 to 30 carbon atoms, a substituted or unsubstituted aryl group having 6 to 30 carbon atoms, a substituted or unsubstituted heteroaryl group having 2 to 50 carbon atoms, a substituted or unsubstituted silyl group, a substituted or unsubstituted germanium group, a cyano group, and a halogen group.

R₃₁ to R₃₅ are the same as or different from each other, and are each independently selected from hydrogen, deuterium, substituted or unsubstituted alkyl group having 1 to 20 carbon atoms, a substituted or unsubstituted alkoxy group having 1 to 20 carbon atoms, a substituted or unsubstituted cycloalkyl group having 3 to 20 carbon atoms, a substituted or unsubstituted heterocycloalkyl group having 3 to 30 carbon atoms, a substituted or unsubstituted aryl group having 6 to 30 carbon atoms, a substituted or unsubstituted heteroaryl group having 2 to 50 carbon atoms, a substituted or unsubstituted silyl group, a substituted or unsubstituted germanium group, a cyano group, and a halogen.

Any one of R₃₁ to R₃₅ is a moiety bonded to L₁ to L₃.

In addition, R₃₂ to R₃₅ may be each linked to each other or an adjacent substituent to additionally form an alicyclic or aromatic monocyclic or polycyclic ring, and therefore, according to an embodiment of the present invention, [Structural Formula 2-2] above may be any one selected from the following [Structural Formula 3].

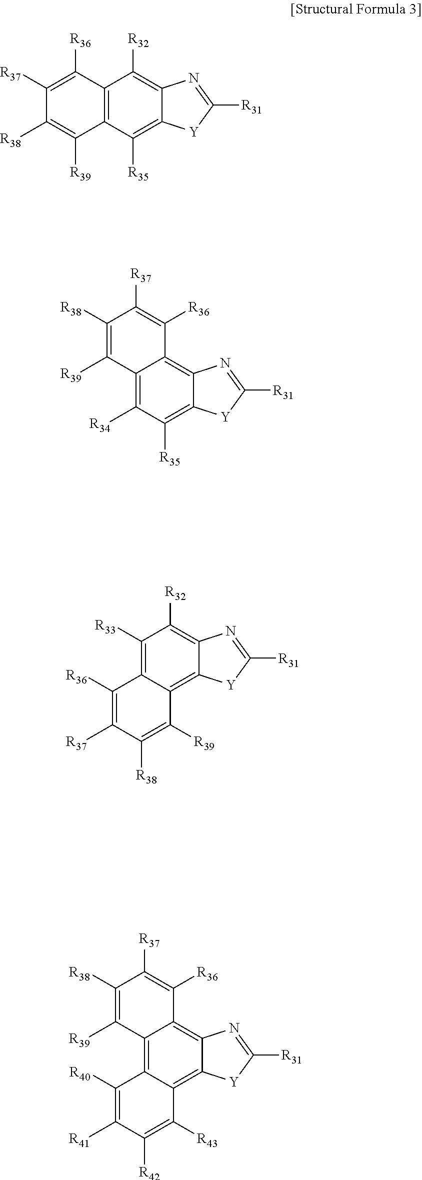

In [Structural Formula 3] above,
Y is selected from S, SO (sulfur monoxide), SO₂ (sulfur dioxide), and NR₂₁, and R₂₁ is selected from hydrogen, deuterium, a substituted or unsubstituted alkyl group having 1 to 20 carbon atoms, a substituted or unsubstituted cycloalkoxy group having 1 to 20 carbon atoms, a substituted or unsubstituted cycloalkyl group having 3 to 20 carbon atoms, a substituted or unsubstituted heterocycloalkyl group having 3 to 30 carbon atoms, a substituted or unsubstituted aryl group having 6 to 30 carbon atoms, a substituted or unsubstituted heteroaryl group having 2 to 50 carbon atoms, a substituted or unsubstituted silyl group, a substituted or unsubstituted germanium group, a cyano group, and a halogen group.

R₃₁ to R₄₃ are the same as or different from each other, and are each independently selected from hydrogen, deuterium, substituted or unsubstituted alkyl group having 1 to 20 carbon atoms, a substituted or unsubstituted alkoxy group having 1 to 20 carbon atoms, a substituted or unsubstituted cycloalkyl group having 3 to 20 carbon atoms, a substituted or unsubstituted heterocycloalkyl group having 3 to 30 carbon atoms, a substituted or unsubstituted aryl group having 6 to 30 carbon atoms, a substituted or unsubstituted heteroaryl group having 2 to 50 carbon atoms, a substituted or unsubstituted silyl group, a substituted or unsubstituted germanium group, a cyano group, and a halogen.

Any one of R₃₁ to R₄₃ is a moiety bonded to L₁ to L₃.

According to an embodiment of the present invention, Ar₁ to Ar₃ may be the same as or different from each other, and may be each independently the following [Structural Formula 1] or [Structural Formula 2].

Meanwhile, the ‘substituted or unsubstituted’ means being substituted with one or two or more substituents selected from deuterium, a cyano group, a halogen group, a hydroxy group, a nitro group, an alkyl group, a halogenated alkyl group, an alkoxy group, a halogenated alkoxy group, a cycloalkyl group, a heterocycloalkyl group, an aryl group, a heteroaryl group, a mixed aliphatic-aromatic cyclic group, an amine group, a silyl group, and a germanium group, being substituted with a substituent to which two or more substituents among the aforementioned substituents are linked, or having no substituent.

For specific examples, the substituted arylene group means that a phenyl group, a biphenyl group, a naphthalene group, a fluorenyl group, a pyrenyl group, a phenanthrenyl group, a perylene group, a tetracenyl group, and an anthracenyl group are substituted with other substituents. In addition, the substituted heteroaryl group means that a pyridyl group, a thiophenyl group, a triazine group, a quinoline group, a phenanthroline group, an imidazole group, a thiazole group, an oxazole group, a carbazole group and a condensate heteroring group thereof, for example, a benzquinoline group, a benzimidazole group, a benzoxazole group, a benzthiazole group, a benzcarbazole group, a dibenzothiophenyl group, and a dibenzofuran group are substituted with other substituents.

As used herein, the expression “bonded to an adjacent group to form a ring” means that the corresponding group combines with an adjacent group to form a substituted or unsubstituted alicyclic or aromatic ring and the term “adjacent substituent” may mean a substituent on an atom directly attached to an atom substituted with the corresponding substituent, a substituent disposed sterically closest to the corresponding substituent or another substituent on an atom substituted with the corresponding substituent. For example, two substituents substituted at the ortho position of a benzene ring or two substituents on the same carbon in an aliphatic ring may be considered “adjacent” to each other.

In an embodiment of the present invention, the alkyl groups may be straight or branched. Specific examples of the alkyl groups include, but are not limited to, methyl, ethyl, propyl, n-propyl, isopropyl, butyl, n-butyl, isobutyl, tert-butyl, sec-butyl, 1-methylbutyl, 1-ethylbutyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethylpropyl, 1,1-dimethylpropyl, isohexyl, 2-methylpentyl, 4-methylhexyl, and 5-methylhexyl groups.

In an embodiment of the present invention, the alkoxy groups may be straight or branched. Specific examples of the alkoxy groups include, but are not limited to, methoxy, ethoxy, n-propoxy, isopropoxy, i-propyloxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, and p-methylbenzyloxy groups.

In an embodiment of the present invention, the deuterated alkyl group or alkoxy group and the halogenated alkyl group or alkoxy group mean an alkyl group or alkoxy group in which the above alkyl group or alkoxy group is substituted with deuterium or a halogen group.

In an embodiment of the present invention, the aromatic hydrocarbon rings or aryl groups may be monocyclic or polycyclic ones. Examples of the monocyclic aryl groups include, but are not limited to, phenyl, biphenyl, terphenyl, and stilbenyl groups. Examples of the polycyclic aryl groups include naphthyl, anthracenyl, phenanthrenyl, pyrenyl, perylenyl, tetracenyl, chrysenyl, fluorenyl, acenaphathcenyl, triphenylene, and fluoranthrene groups but the scope of the present invention is not limited thereto.

In addition, in an embodiment of the present invention, the fluorenyl groups refer to structures in which two cyclic organic compounds are linked through one atom, and examples thereof include

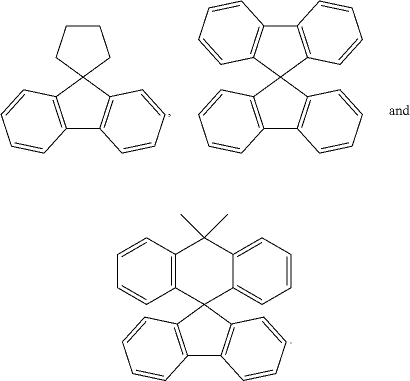

In an embodiment of the present invention, the fluorenyl groups include open structures in which one of the two cyclic organic compounds linked through one atom is cleaved, and examples thereof include

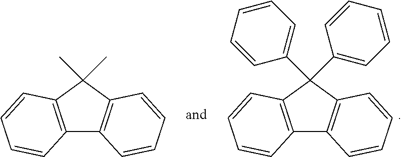

In addition, carbon atoms of the ring may be substituted with any one or more heteroatoms selected from among N, S and O, and examples thereof include

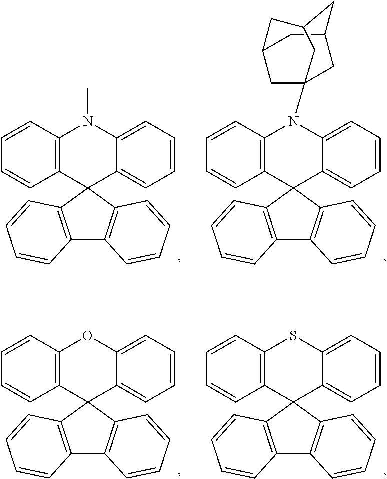

and the like.

In addition, carbon atoms of the ring may be substituted with any one or more heteroatoms selected from among N, S and O, and examples thereof include

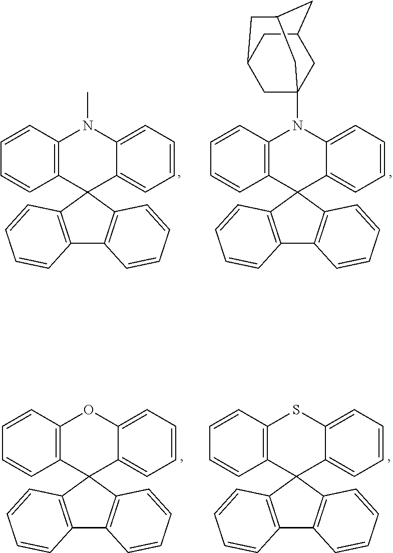

and the like.

In an embodiment of the present invention, the aromatic heterocyclic rings or heteroaryl groups refer to aromatic groups containing one or more heteroatoms. Examples of the aromatic heterocyclic rings or heteroaryl groups include, but are not limited to, thiophene, furan, pyrrole, imidazole, thiazole, oxazole, oxadiazole, triazole, pyridyl, bipyridyl, pyrimidyl, triazine, triazole, acridyl, pyridazine, pyrazinyl, quinolinyl, quinazoline, quinoxalinyl, phthalazinyl, pyridopyrimidinyl, pyridopyrazinyl, pyrazinopyrazinyl, isoquinoline, indole, carbazole, benzoxazole, benzimidazole, benzothiazole, benzocarbazole, benzothiophene, dibenzothiophene, benzofuranyl, dibenzofuranyl, phenanthroline, thiazolyl, isoxazolyl, oxadiazolyl, thiadiazolyl, benzothiazolyl, and phenothiazinyl groups.

In an embodiment of the present invention, the aliphatic hydrocarbon rings or cycloalkyl groups refer to non-aromatic rings consisting only of carbon and hydrogen atoms. The aliphatic hydrocarbon ring is intended to include monocyclic and polycyclic ones and may be optionally substituted with one or more other substituents. As used herein, the term “polycyclic” means that the aliphatic hydrocarbon ring may be directly attached or fused to one or more other cyclic groups. The other cyclic groups may be aliphatic hydrocarbon rings and other examples thereof include aliphatic heterocyclic, aryl, and heteroaryl groups. Specific examples of the aliphatic hydrocarbon rings include, but are not limited to, cycloalkyl groups such as cyclopropyl, cyclobutyl, cyclopentyl, adamantyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, and cyclooctyl, cycloalkanes such as cyclohexane and cyclopentane, and cycloalkenes such as cyclohexene and cyclobutene.

In an embodiment of the present invention, the aliphatic heterocyclic rings or heterocycloalkyl groups refer to aliphatic rings containing one or more heteroatoms such as O, S, Se, N, and Si. The aliphatic heterocyclic ring is intended to include monocyclic or polycyclic ones and may be optionally substituted with one or more other substituents. As used herein, the term “polycyclic” means that the aliphatic heterocyclic ring such as heterocycloalkyl, heterocycloalkane or heterocycloalkene may be directly attached or fused to one or more other cyclic groups. The other cyclic groups may be aliphatic heterocyclic rings and other examples thereof include aliphatic hydrocarbon rings, aryl groups, and heteroaryl groups.

In an embodiment of the present invention, the mixed aliphatic-aromatic cyclic groups refer to structures in which at least one aliphatic ring and at least one aromatic ring are linked or fused together and which are overall non-aromatic. The mixed aliphatic-aromatic polycyclic rings may contain one or more heteroatoms selected from N, O, P, and S other than carbon atoms (C).

In an embodiment of the present invention, the silyl group is intended to include —SiH₃, alkylsilyl, arylsilyl, alkylarylsilyl, arylheteroarylsilyl, and heteroarylsilyl. The arylsilyl refers to a silyl group obtained by substituting one, two or three of the hydrogen atoms in —SiH₃ with aryl groups. The alkylsilyl refers to a silyl group obtained by substituting one, two or three of the hydrogen atoms in —SiH₃ with alkyl groups. The alkylarylsilyl refers to a silyl group obtained by substituting one of the hydrogen atoms in —SiH₃ with an alkyl group and the other two hydrogen atoms with aryl groups or substituting two of the hydrogen atoms in —SiH₃ with alkyl groups and the remaining hydrogen atom with an aryl group. The arylheteroarylsilyl refers to a silyl group obtained by substituting one of the hydrogen atoms in —SiH₃ with an aryl group and the other two hydrogen atoms with heteroaryl groups or substituting two of the hydrogen atoms in —SiH₃ with aryl groups and the remaining hydrogen atom with a heteroaryl group. The heteroarylsilyl refers to a silyl group obtained by substituting one, two or three of the hydrogen atoms in —SiH₃ with heteroaryl groups. The arylsilyl group may be, for example, substituted or unsubstituted monoarylsilyl, substituted or unsubstituted diarylsilyl, or substituted or unsubstituted triarylsilyl. The same applies to the alkylsilyl and heteroarylsilyl groups.

Each of the aryl groups in the arylsilyl, heteroarylsilyl, and arylheteroarylsilyl groups may be a monocyclic or polycyclic one. Each of the heteroaryl groups in the arylsilyl, heteroarylsilyl, and arylheteroarylsilyl groups may be a monocyclic or polycyclic one.

Specific examples of the silyl groups include trimethylsilyl, triethylsilyl, triphenylsilyl, trimethoxysilyl, dimethoxyphenylsilyl, diphenylmethylsilyl, diphenylvinylsilyl, methylcyclobutylsilyl, and dimethylfurylsilyl. One or more of the hydrogen atoms in each of the silyl groups may be substituted with the substituents mentioned in the aryl groups.

In the present invention, the germanium group is intended to include —GeH₃, alkylgermanium, arylgermanium, heteroarylgermanium, alkylarylgermanium, alkylheteroarylgermanium, and arylheteroarylgermanium. The definitions of the substituents in the germanium groups follow those described for the silyl groups, except that the silicon (Si) atom in each silyl group is changed to a germanium (Ge) atom.

Specific examples of the germanium groups include trimethylgermane, triethylgermane, triphenylgermane, trimethoxygermane, dimethoxyphenylgermane, diphenylmethylgermane, diphenylvinylgermane, methylcyclobutylgermane, and dimethylfurylgermane. One or more of the hydrogen atoms in each of the germanium groups may be substituted with the substituents mentioned in the aryl groups.

In the present invention, the amine group may be —NH₂, an alkylamine group, an arylamine group, an arylheteroarylamine group, etc., the arylamine group refers to amine substituted with an aryl group, the alkylamine group refers to amine substituted with an alkyl group, and the arylheteroarylamine group refers to amine substituted with aryl and heteroaryl groups. Examples of the arylamine group include a substituted or unsubstituted monoarylamine group, a substituted or unsubstituted diarylamine group, or a substituted or unsubstituted triarylamine group. The aryl group and the heteroaryl group in the arylamine group and the arylheteroarylamine group may be a monocyclic aryl group, a monocyclic heteroaryl group, a polycyclic aryl group, or a polycyclic heteroaryl group, and the arylamine group and the arylheteroarylamine group including two or more aryl groups and heteroaryl groups may include a monocyclic aryl group (heteroaryl group), a polycyclic aryl group (heteroaryl group), or both a monocyclic aryl group (heteroaryl group) and a polycyclic aryl group (heteroaryl group). In addition, the aryl group and the heteroaryl group in the arylamine group and the arylheteroarylamine group may be selected from examples of the above-mentioned aryl group and heteroaryl group.

In the present invention, the halogen group may be, for example, fluorine, chlorine, bromine or iodine.

Furthermore, various specific examples of the substituent according to the present invention can be clearly confirmed in the specific compounds described below.

The organic compound according to the present invention represented by [Formula I] may be used as a material for the light efficiency improving layer (capping layer) provided in the organic light emitting device due to its structural specificity.

Preferred specific examples of the organic compound represented by [Formula I] according to the present invention include the following compounds but are not limited thereto.

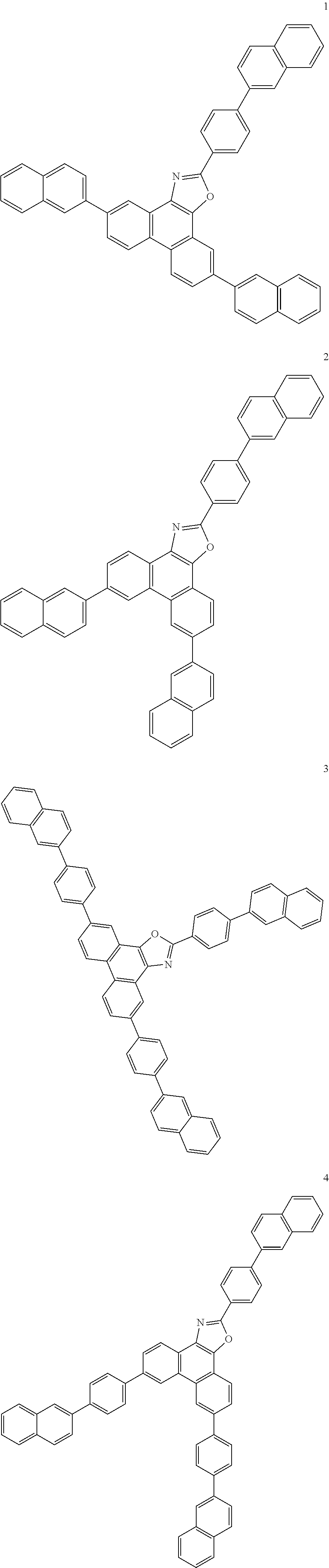 [SMILES: c1ccc2cc(-c3ccc(-c4nc5c6cc(-c7ccc8ccccc8c7)ccc6c6ccc(-c7ccc8ccccc8c7)cc6c5o4)cc3)ccc2c1 ; c1ccc2cc(-c3ccc(-c4nc5c6ccc(-c7ccc8ccccc8c7)cc6c6cc(-c7ccc8ccccc8c7)ccc6c5o4)cc3)ccc2c1 ; c1ccc2cc(-c3ccc(-c4ccc5c6ccc(-c7ccc(-c8ccc9ccccc9c8)cc7)cc6c6oc(-c7ccc(-c8ccc9ccccc9c8)cc7)nc6c5c4)cc3)ccc2c1 ; c1ccc2cc(-c3ccc(-c4ccc5c(c4)c4cc(-c6ccc(-c7ccc8ccccc8c7)cc6)ccc4c4oc(-c6ccc(-c7ccc8ccccc8c7)cc6)nc54)cc3)ccc2c1]

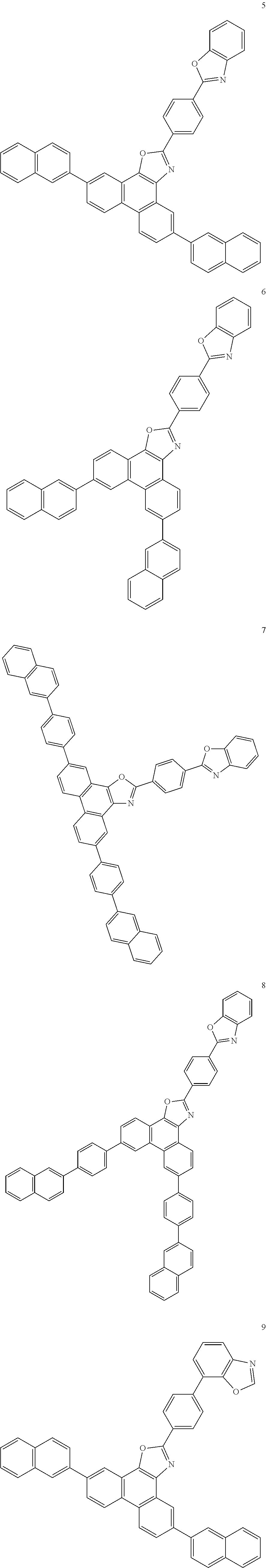

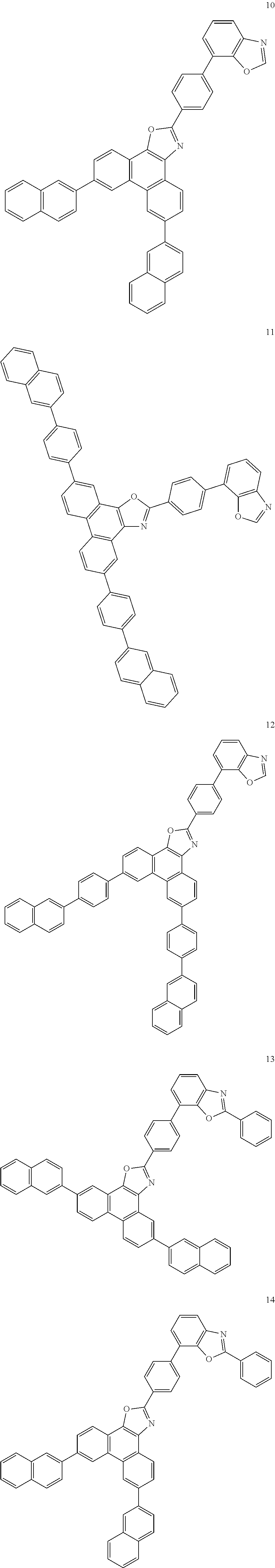

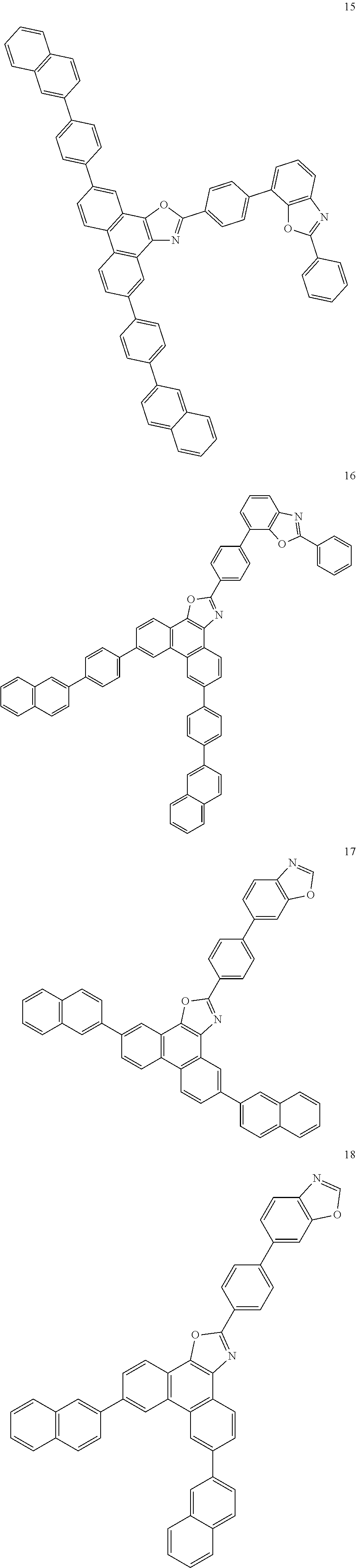 [SMILES: c1ccc(-c2nc3cccc(-c4ccc(-c5nc6c7cc(-c8ccc(-c9ccc%10ccccc%10c9)cc8)ccc7c7ccc(-c8ccc(-c9ccc%10ccccc%10c9)cc8)cc7c6o5)cc4)c3o2)cc1 ; c1ccc(-c2nc3cccc(-c4ccc(-c5nc6c7ccc(-c8ccc(-c9ccc%10ccccc%10c9)cc8)cc7c7cc(-c8ccc(-c9ccc%10ccccc%10c9)cc8)ccc7c6o5)cc4)c3o2)cc1 ; c1ccc2cc(-c3ccc4c5ccc(-c6ccc7ccccc7c6)cc5c5oc(-c6ccc(-c7ccc8ncoc8c7)cc6)nc5c4c3)ccc2c1 ; c1ccc2cc(-c3ccc4c(c3)c3cc(-c5ccc6ccccc6c5)ccc3c3oc(-c5ccc(-c6ccc7ncoc7c6)cc5)nc43)ccc2c1]

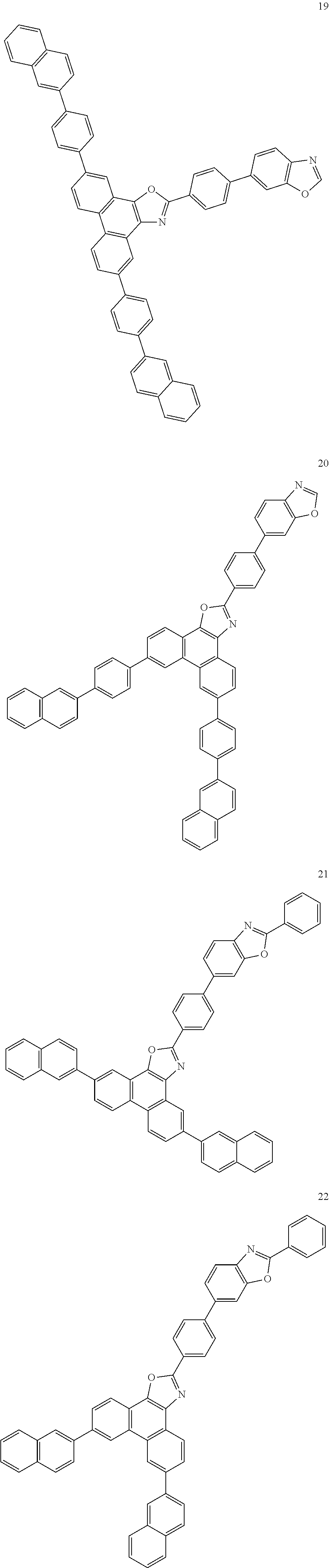 [SMILES: c1ccc2cc(-c3ccc(-c4ccc5c6ccc(-c7ccc(-c8ccc9ccccc9c8)cc7)cc6c6oc(-c7ccc(-c8ccc9ncoc9c8)cc7)nc6c5c4)cc3)ccc2c1 ; c1ccc2cc(-c3ccc(-c4ccc5c(c4)c4cc(-c6ccc(-c7ccc8ccccc8c7)cc6)ccc4c4oc(-c6ccc(-c7ccc8ncoc8c7)cc6)nc54)cc3)ccc2c1 ; c1ccc(-c2nc3ccc(-c4ccc(-c5nc6c7cc(-c8ccc9ccccc9c8)ccc7c7ccc(-c8ccc9ccccc9c8)cc7c6o5)cc4)cc3o2)cc1 ; c1ccc(-c2nc3ccc(-c4ccc(-c5nc6c7ccc(-c8ccc9ccccc9c8)cc7c7cc(-c8ccc9ccccc9c8)ccc7c6o5)cc4)cc3o2)cc1]

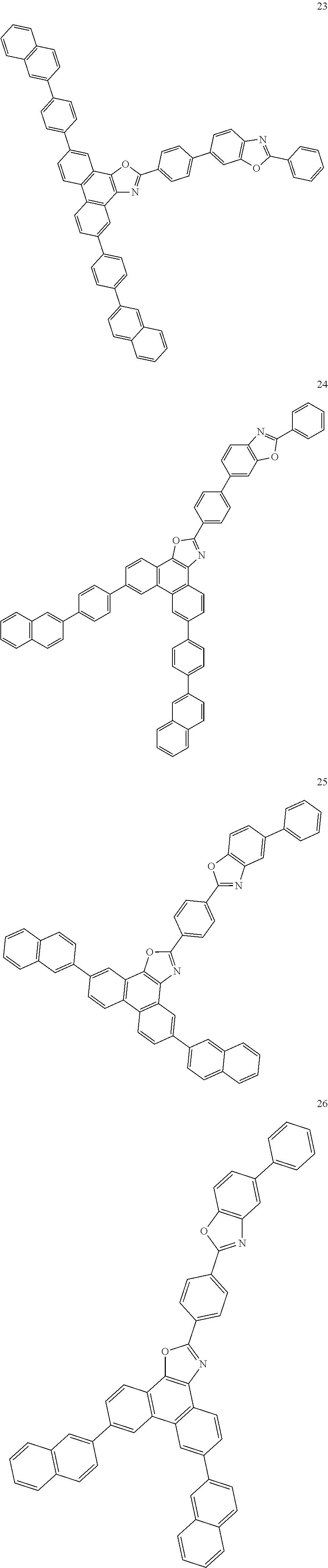 [SMILES: c1ccc(-c2nc3ccc(-c4ccc(-c5nc6c7cc(-c8ccc(-c9ccc%10ccccc%10c9)cc8)ccc7c7ccc(-c8ccc(-c9ccc%10ccccc%10c9)cc8)cc7c6o5)cc4)cc3o2)cc1 ; c1ccc(-c2nc3ccc(-c4ccc(-c5nc6c7ccc(-c8ccc(-c9ccc%10ccccc%10c9)cc8)cc7c7cc(-c8ccc(-c9ccc%10ccccc%10c9)cc8)ccc7c6o5)cc4)cc3o2)cc1 ; c1ccc(-c2ccc3oc(-c4ccc(-c5nc6c7cc(-c8ccc9ccccc9c8)ccc7c7ccc(-c8ccc9ccccc9c8)cc7c6o5)cc4)nc3c2)cc1 ; c1ccc(-c2ccc3oc(-c4ccc(-c5nc6c7ccc(-c8ccc9ccccc9c8)cc7c7cc(-c8ccc9ccccc9c8)ccc7c6o5)cc4)nc3c2)cc1]

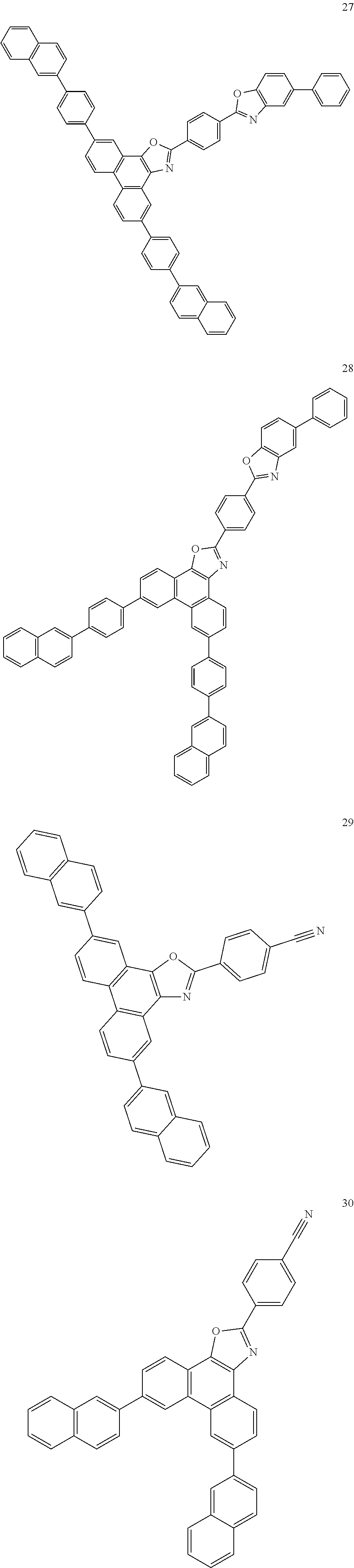 [SMILES: c1ccc(-c2ccc3oc(-c4ccc(-c5nc6c7cc(-c8ccc(-c9ccc%10ccccc%10c9)cc8)ccc7c7ccc(-c8ccc(-c9ccc%10ccccc%10c9)cc8)cc7c6o5)cc4)nc3c2)cc1 ; c1ccc(-c2ccc3oc(-c4ccc(-c5nc6c7ccc(-c8ccc(-c9ccc%10ccccc%10c9)cc8)cc7c7cc(-c8ccc(-c9ccc%10ccccc%10c9)cc8)ccc7c6o5)cc4)nc3c2)cc1 ; N#Cc1ccc(-c2nc3c4cc(-c5ccc6ccccc6c5)ccc4c4ccc(-c5ccc6ccccc6c5)cc4c3o2)cc1 ; N#Cc1ccc(-c2nc3c4ccc(-c5ccc6ccccc6c5)cc4c4cc(-c5ccc6ccccc6c5)ccc4c3o2)cc1]

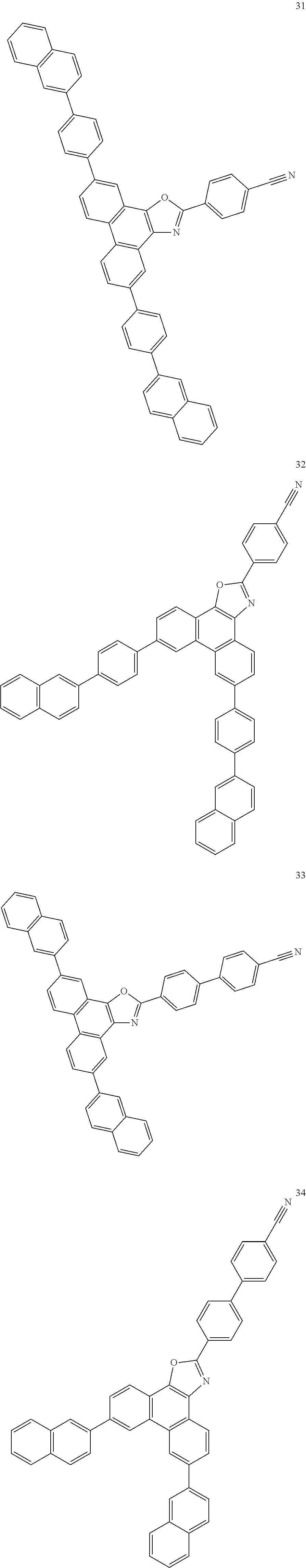 [SMILES: N#Cc1ccc(-c2nc3c4cc(-c5ccc(-c6ccc7ccccc7c6)cc5)ccc4c4ccc(-c5ccc(-c6ccc7ccccc7c6)cc5)cc4c3o2)cc1 ; N#Cc1ccc(-c2nc3c4ccc(-c5ccc(-c6ccc7ccccc7c6)cc5)cc4c4cc(-c5ccc(-c6ccc7ccccc7c6)cc5)ccc4c3o2)cc1 ; N#Cc1ccc(-c2ccc(-c3nc4c5cc(-c6ccc7ccccc7c6)ccc5c5ccc(-c6ccc7ccccc7c6)cc5c4o3)cc2)cc1 ; N#Cc1ccc(-c2ccc(-c3nc4c5ccc(-c6ccc7ccccc7c6)cc5c5cc(-c6ccc7ccccc7c6)ccc5c4o3)cc2)cc1]

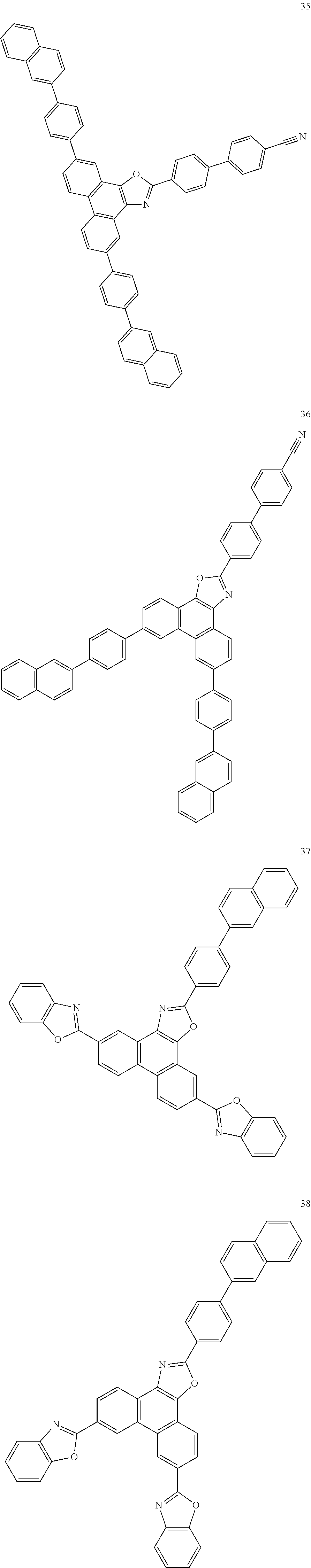 [SMILES: N#Cc1ccc(-c2ccc(-c3nc4c5cc(-c6ccc(-c7ccc8ccccc8c7)cc6)ccc5c5ccc(-c6ccc(-c7ccc8ccccc8c7)cc6)cc5c4o3)cc2)cc1 ; N#Cc1ccc(-c2ccc(-c3nc4c5ccc(-c6ccc(-c7ccc8ccccc8c7)cc6)cc5c5cc(-c6ccc(-c7ccc8ccccc8c7)cc6)ccc5c4o3)cc2)cc1 ; c1ccc2cc(-c3ccc(-c4nc5c6cc(-c7nc8ccccc8o7)ccc6c6ccc(-c7nc8ccccc8o7)cc6c5o4)cc3)ccc2c1 ; c1ccc2cc(-c3ccc(-c4nc5c6ccc(-c7nc8ccccc8o7)cc6c6cc(-c7nc8ccccc8o7)ccc6c5o4)cc3)ccc2c1]

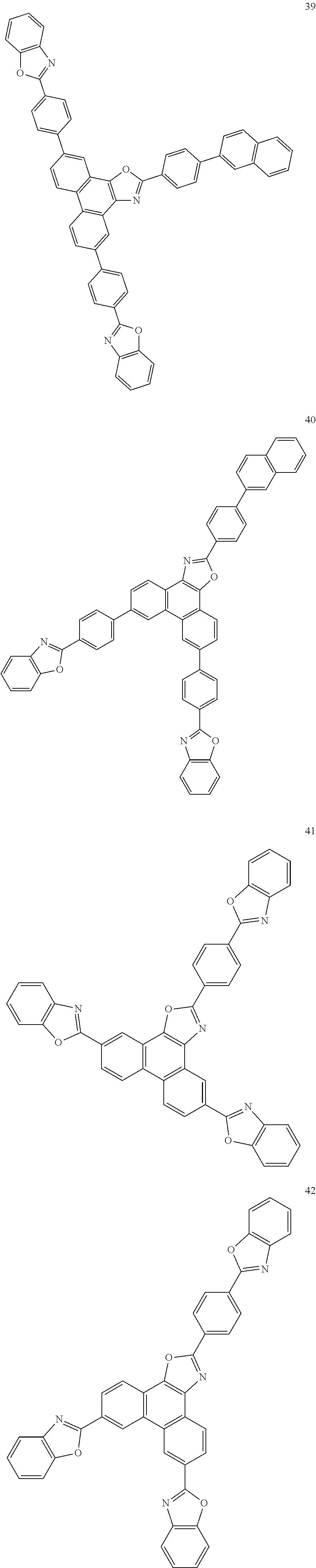 [SMILES: c1ccc2cc(-c3ccc(-c4nc5c6cc(-c7ccc(-c8nc9ccccc9o8)cc7)ccc6c6ccc(-c7ccc(-c8nc9ccccc9o8)cc7)cc6c5o4)cc3)ccc2c1 ; c1ccc2cc(-c3ccc(-c4nc5c6ccc(-c7ccc(-c8nc9ccccc9o8)cc7)cc6c6cc(-c7ccc(-c8nc9ccccc9o8)cc7)ccc6c5o4)cc3)ccc2c1 ; c1ccc2oc(-c3ccc(-c4nc5c6cc(-c7nc8ccccc8o7)ccc6c6ccc(-c7nc8ccccc8o7)cc6c5o4)cc3)nc2c1 ; c1ccc2oc(-c3ccc(-c4nc5c6ccc(-c7nc8ccccc8o7)cc6c6cc(-c7nc8ccccc8o7)ccc6c5o4)cc3)nc2c1]

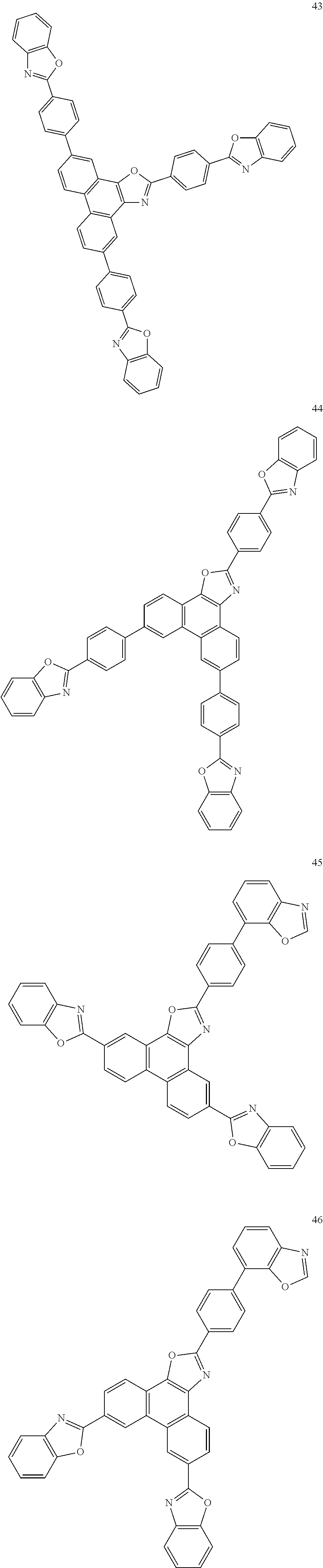 [SMILES: c1ccc2oc(-c3ccc(-c4ccc5c6ccc(-c7ccc(-c8nc9ccccc9o8)cc7)cc6c6oc(-c7ccc(-c8nc9ccccc9o8)cc7)nc6c5c4)cc3)nc2c1 ; c1ccc2oc(-c3ccc(-c4ccc5c(c4)c4cc(-c6ccc(-c7nc8ccccc8o7)cc6)ccc4c4oc(-c6ccc(-c7nc8ccccc8o7)cc6)nc54)cc3)nc2c1 ; c1ccc2oc(-c3ccc4c5ccc(-c6nc7ccccc7o6)cc5c5oc(-c6ccc(-c7cccc8ncoc78)cc6)nc5c4c3)nc2c1 ; c1ccc2oc(-c3ccc4c(c3)c3cc(-c5nc6ccccc6o5)ccc3c3oc(-c5ccc(-c6cccc7ncoc67)cc5)nc43)nc2c1]

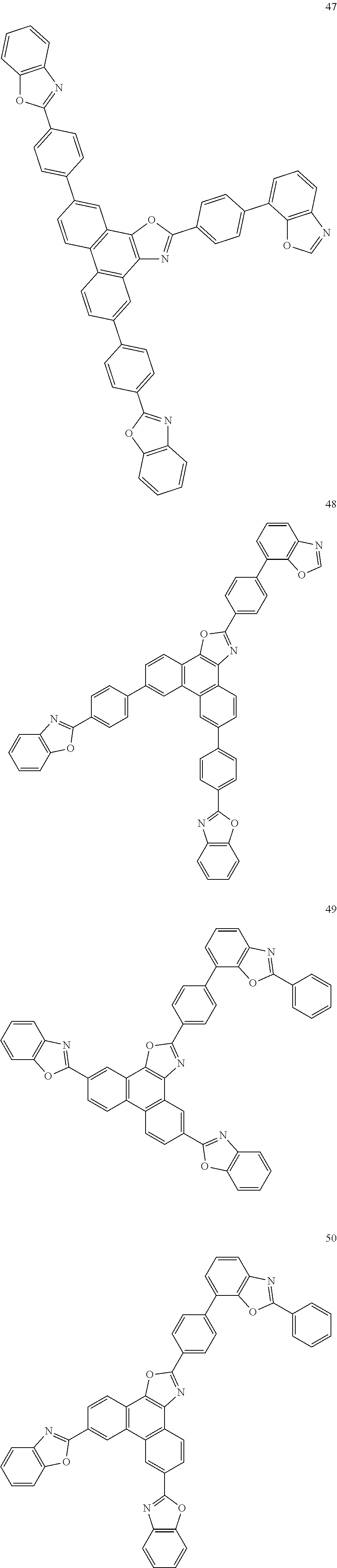 [SMILES: c1ccc2oc(-c3ccc(-c4ccc5c6ccc(-c7ccc(-c8nc9ccccc9o8)cc7)cc6c6oc(-c7ccc(-c8cccc9ncoc89)cc7)nc6c5c4)cc3)nc2c1 ; c1ccc2oc(-c3ccc(-c4ccc5c(c4)c4cc(-c6ccc(-c7nc8ccccc8o7)cc6)ccc4c4oc(-c6ccc(-c7cccc8ncoc78)cc6)nc54)cc3)nc2c1 ; c1ccc(-c2nc3cccc(-c4ccc(-c5nc6c7cc(-c8nc9ccccc9o8)ccc7c7ccc(-c8nc9ccccc9o8)cc7c6o5)cc4)c3o2)cc1 ; c1ccc(-c2nc3cccc(-c4ccc(-c5nc6c7ccc(-c8nc9ccccc9o8)cc7c7cc(-c8nc9ccccc9o8)ccc7c6o5)cc4)c3o2)cc1]

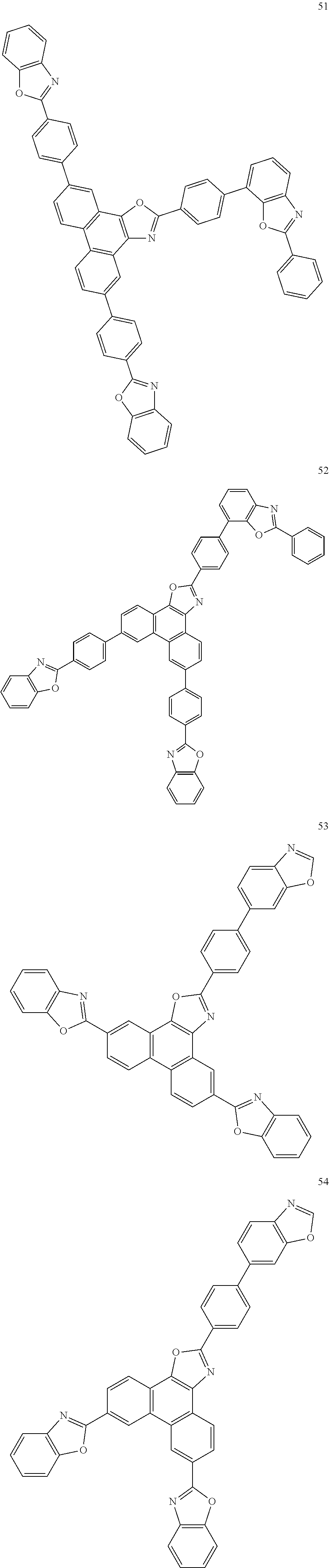 [SMILES: c1ccc(-c2nc3cccc(-c4ccc(-c5nc6c7cc(-c8ccc(-c9nc%10ccccc%10o9)cc8)ccc7c7ccc(-c8ccc(-c9nc%10ccccc%10o9)cc8)cc7c6o5)cc4)c3o2)cc1 ; c1ccc(-c2nc3cccc(-c4ccc(-c5nc6c7ccc(-c8ccc(-c9nc%10ccccc%10o9)cc8)cc7c7cc(-c8ccc(-c9nc%10ccccc%10o9)cc8)ccc7c6o5)cc4)c3o2)cc1 ; c1ccc2oc(-c3ccc4c5ccc(-c6nc7ccccc7o6)cc5c5oc(-c6ccc(-c7ccc8ncoc8c7)cc6)nc5c4c3)nc2c1 ; c1ccc2oc(-c3ccc4c(c3)c3cc(-c5nc6ccccc6o5)ccc3c3oc(-c5ccc(-c6ccc7ncoc7c6)cc5)nc43)nc2c1]

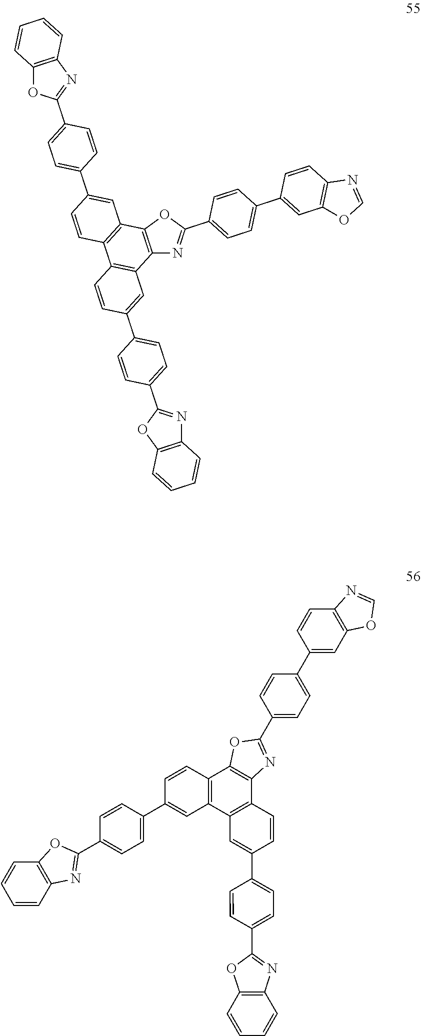 [SMILES: c1ccc2oc(-c3ccc(-c4ccc5c6ccc(-c7ccc(-c8nc9ccccc9o8)cc7)cc6c6oc(-c7ccc(-c8ccc9ncoc9c8)cc7)nc6c5c4)cc3)nc2c1 ; c1ccc2oc(-c3ccc(-c4ccc5c(c4)c4cc(-c6ccc(-c7nc8ccccc8o7)cc6)ccc4c4oc(-c6ccc(-c7ccc8ncoc8c7)cc6)nc54)cc3)nc2c1]

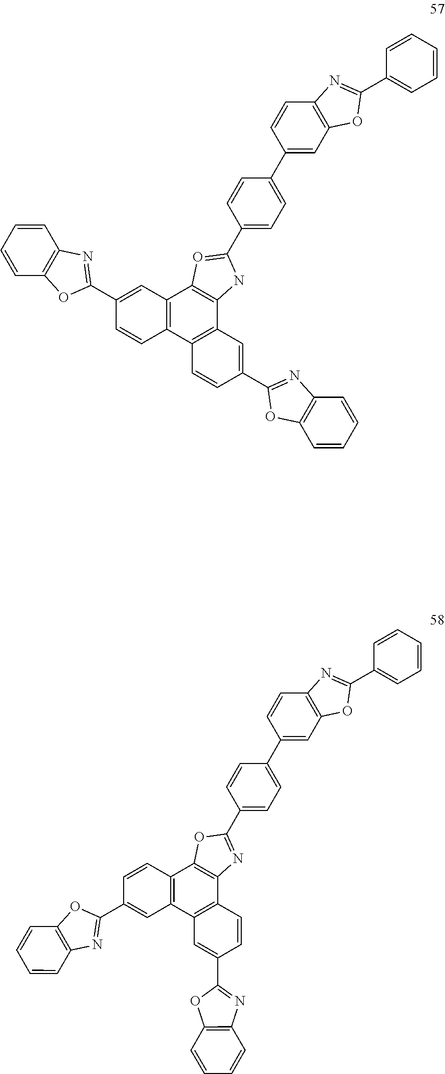

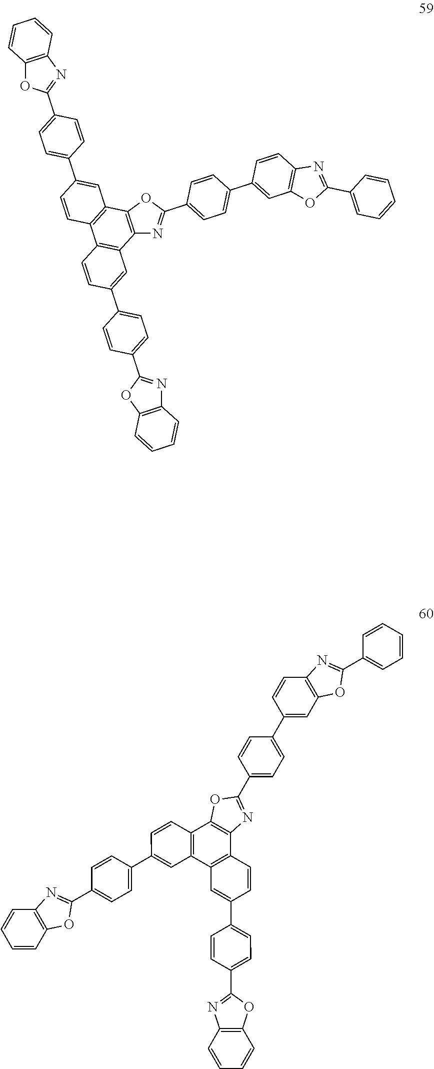 [SMILES: c1ccc(-c2nc3ccc(-c4ccc(-c5nc6c7cc(-c8ccc(-c9nc%10ccccc%10o9)cc8)ccc7c7ccc(-c8ccc(-c9nc%10ccccc%10o9)cc8)cc7c6o5)cc4)cc3o2)cc1 ; c1ccc(-c2nc3ccc(-c4ccc(-c5nc6c7ccc(-c8ccc(-c9nc%10ccccc%10o9)cc8)cc7c7cc(-c8ccc(-c9nc%10ccccc%10o9)cc8)ccc7c6o5)cc4)cc3o2)cc1]

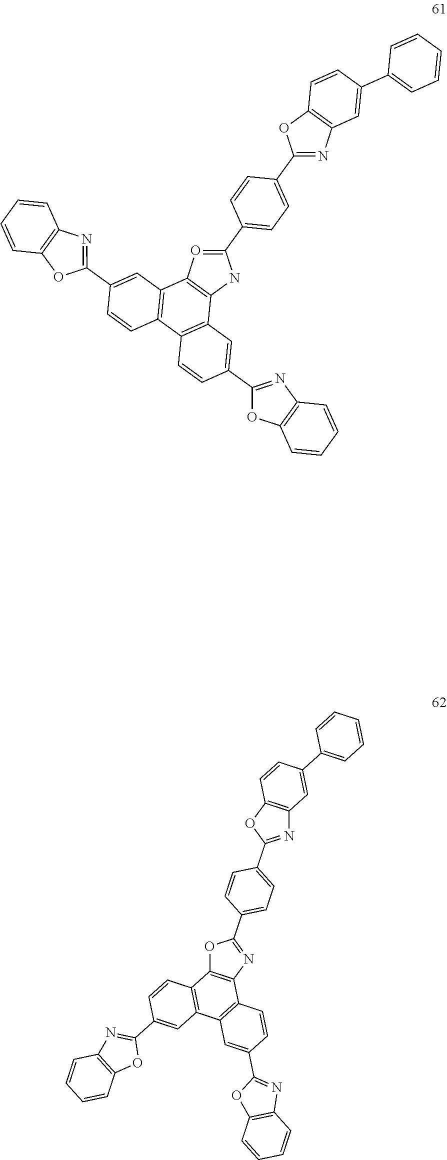

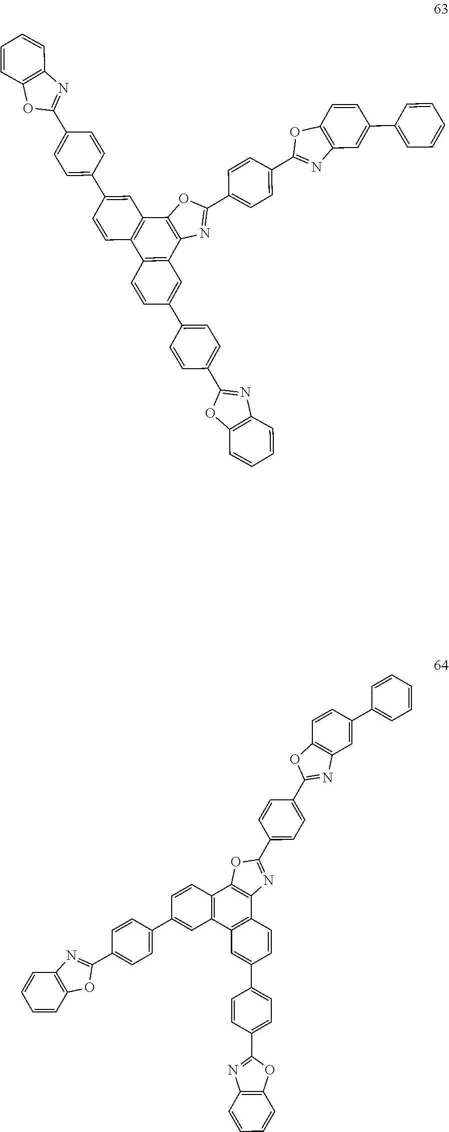 [SMILES: c1ccc(-c2ccc3oc(-c4ccc(-c5nc6c7cc(-c8ccc(-c9nc%10ccccc%10o9)cc8)ccc7c7ccc(-c8ccc(-c9nc%10ccccc%10o9)cc8)cc7c6o5)cc4)nc3c2)cc1 ; c1ccc(-c2ccc3oc(-c4ccc(-c5nc6c7ccc(-c8ccc(-c9nc%10ccccc%10o9)cc8)cc7c7cc(-c8ccc(-c9nc%10ccccc%10o9)cc8)ccc7c6o5)cc4)nc3c2)cc1]

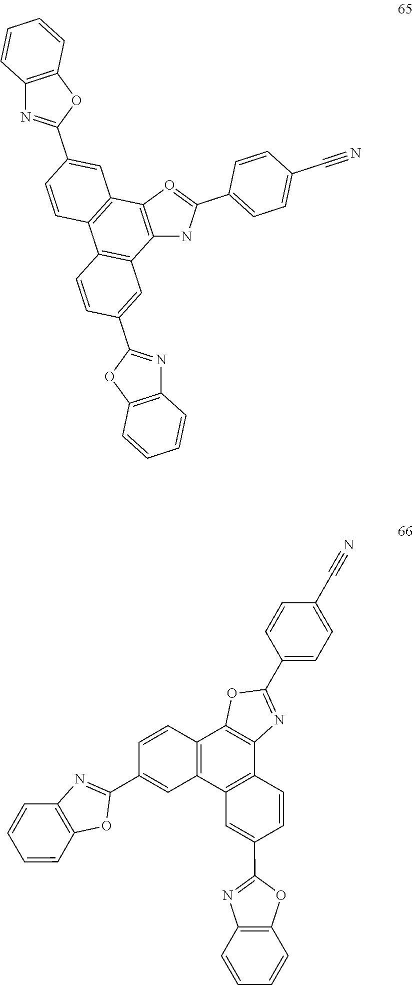

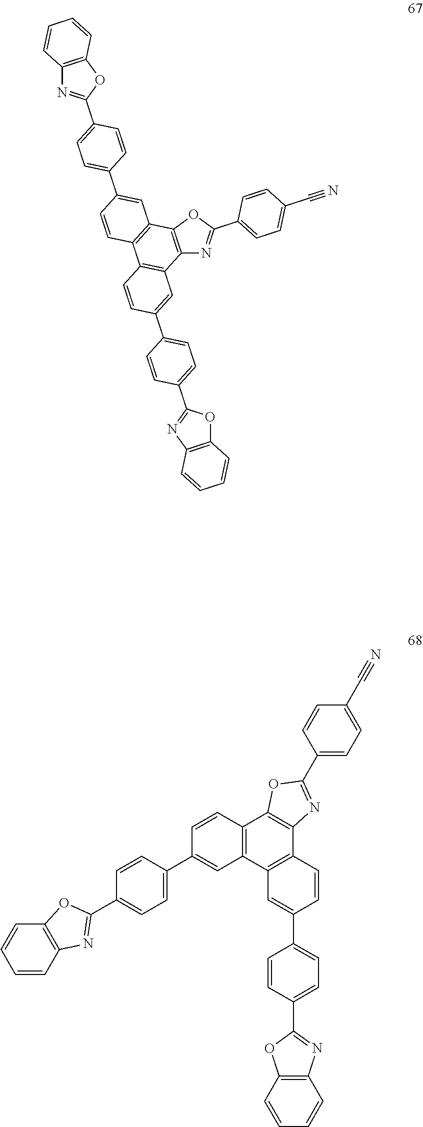 [SMILES: N#Cc1ccc(-c2nc3c4cc(-c5ccc(-c6nc7ccccc7o6)cc5)ccc4c4ccc(-c5ccc(-c6nc7ccccc7o6)cc5)cc4c3o2)cc1 ; N#Cc1ccc(-c2nc3c4ccc(-c5ccc(-c6nc7ccccc7o6)cc5)cc4c4cc(-c5ccc(-c6nc7ccccc7o6)cc5)ccc4c3o2)cc1]

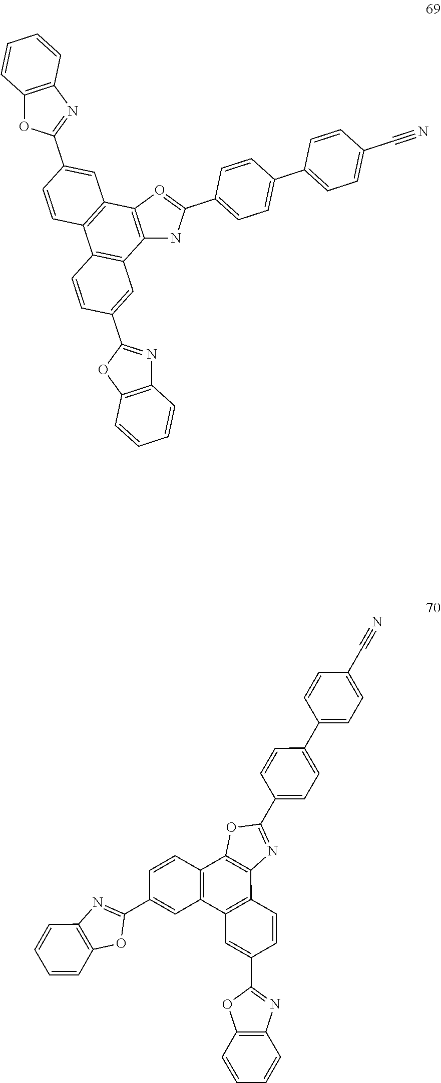

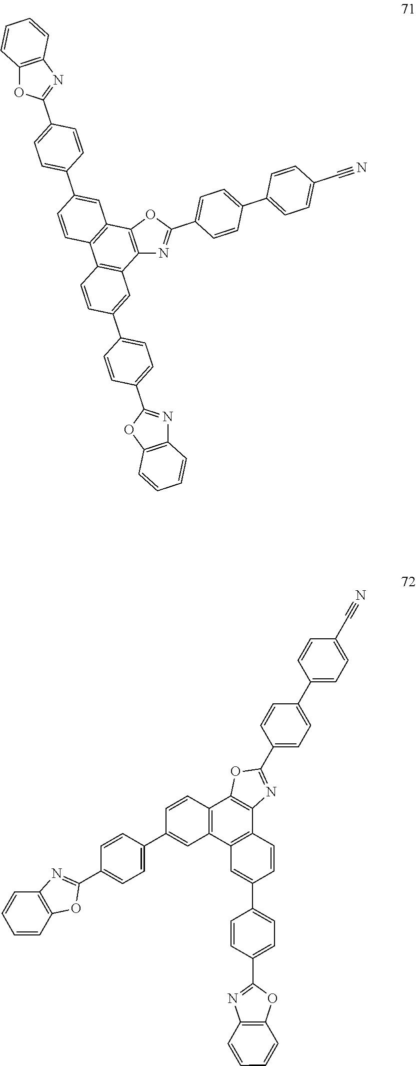 [SMILES: N#Cc1ccc(-c2ccc(-c3nc4c5cc(-c6ccc(-c7nc8ccccc8o7)cc6)ccc5c5ccc(-c6ccc(-c7nc8ccccc8o7)cc6)cc5c4o3)cc2)cc1 ; N#Cc1ccc(-c2ccc(-c3nc4c5ccc(-c6ccc(-c7nc8ccccc8o7)cc6)cc5c5cc(-c6ccc(-c7nc8ccccc8o7)cc6)ccc5c4o3)cc2)cc1]

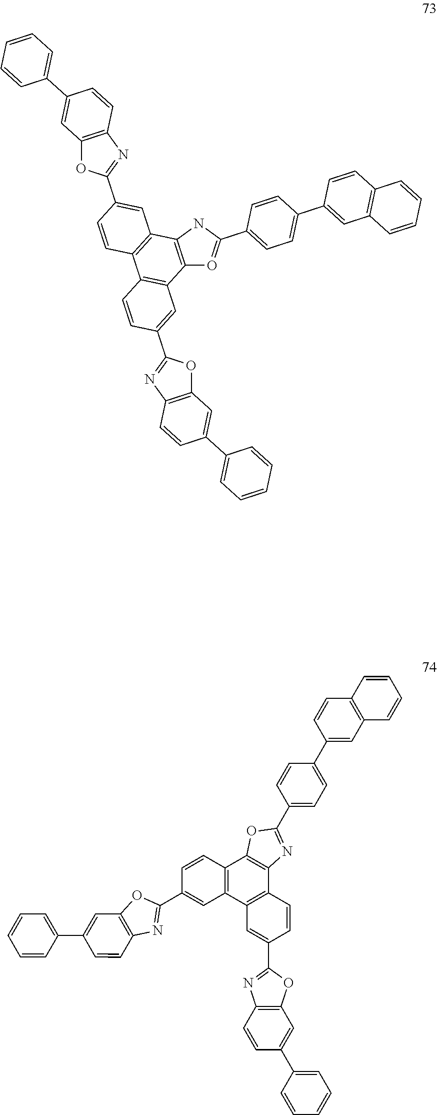

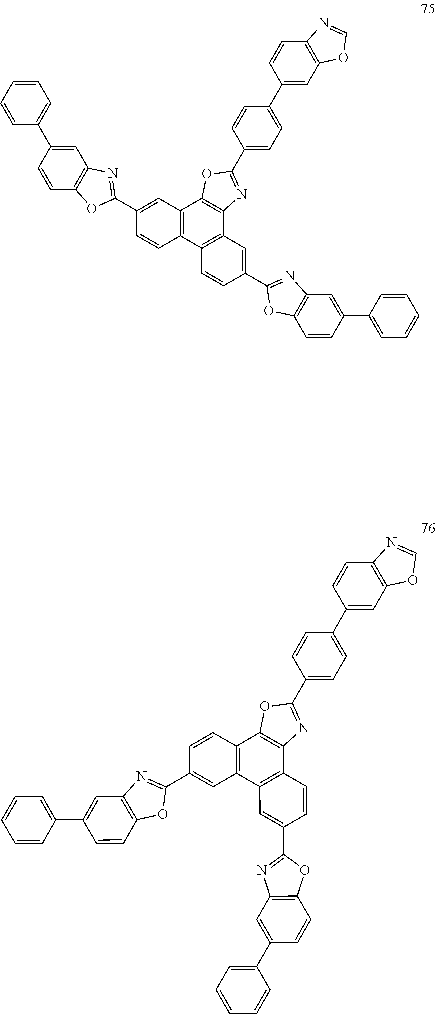 [SMILES: c1ccc(-c2ccc3oc(-c4ccc5c6ccc(-c7nc8cc(-c9ccccc9)ccc8o7)cc6c6oc(-c7ccc(-c8ccc9ncoc9c8)cc7)nc6c5c4)nc3c2)cc1 ; c1ccc(-c2ccc3oc(-c4ccc5c(c4)c4cc(-c6nc7cc(-c8ccccc8)ccc7o6)ccc4c4oc(-c6ccc(-c7ccc8ncoc8c7)cc6)nc54)nc3c2)cc1]

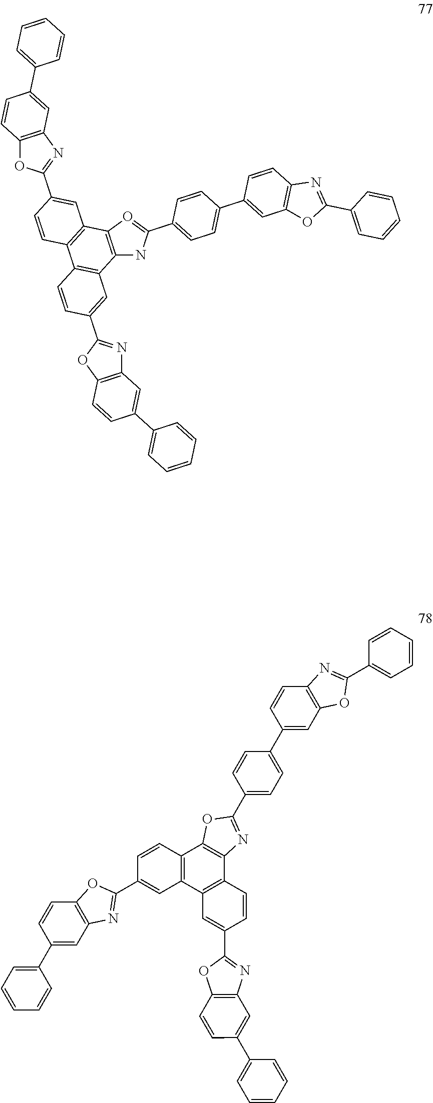

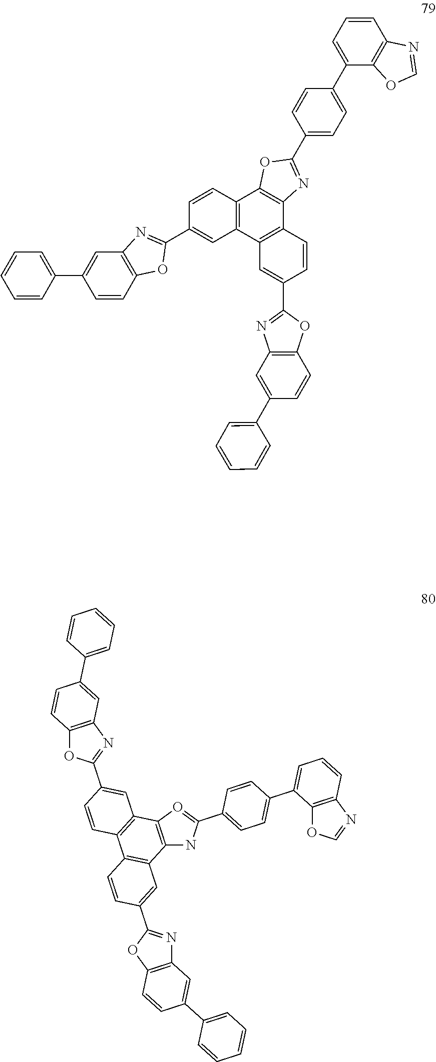

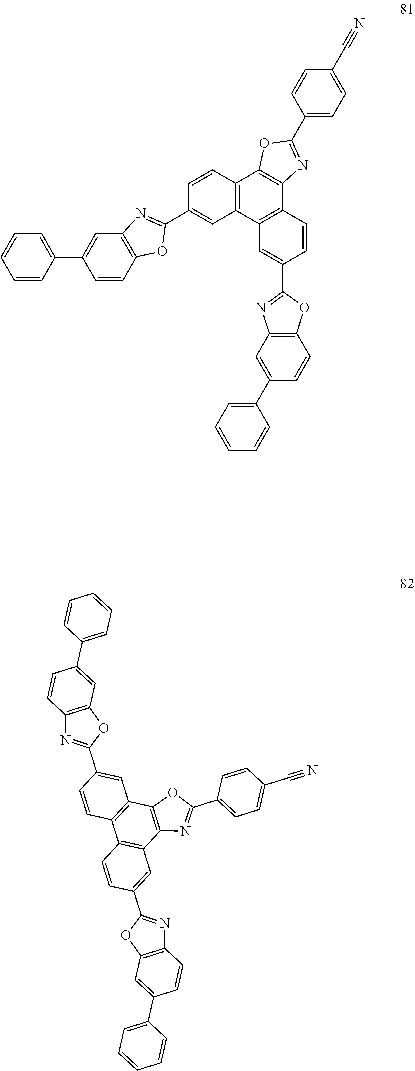 [SMILES: N#Cc1ccc(-c2nc3c4ccc(-c5nc6cc(-c7ccccc7)ccc6o5)cc4c4cc(-c5nc6cc(-c7ccccc7)ccc6o5)ccc4c3o2)cc1 ; N#Cc1ccc(-c2nc3c4cc(-c5nc6ccc(-c7ccccc7)cc6o5)ccc4c4ccc(-c5nc6ccc(-c7ccccc7)cc6o5)cc4c3o2)cc1]

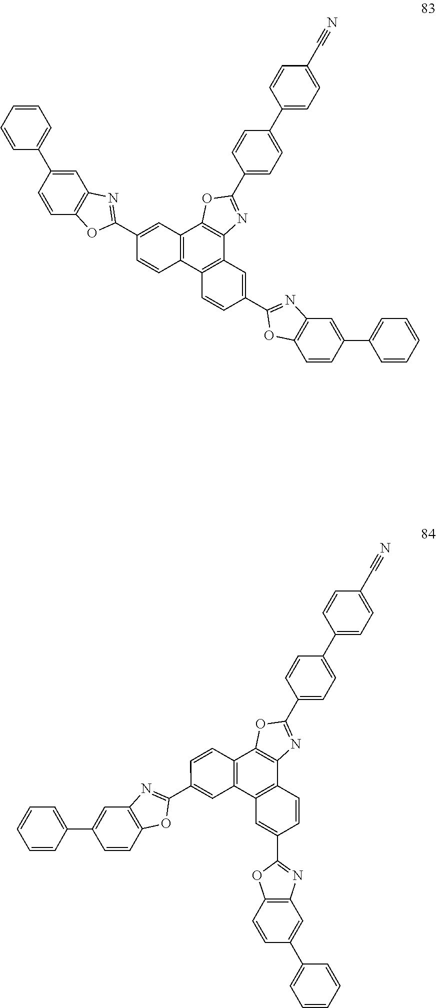 [SMILES: N#Cc1ccc(-c2ccc(-c3nc4c5cc(-c6nc7cc(-c8ccccc8)ccc7o6)ccc5c5ccc(-c6nc7cc(-c8ccccc8)ccc7o6)cc5c4o3)cc2)cc1 ; N#Cc1ccc(-c2ccc(-c3nc4c5ccc(-c6nc7cc(-c8ccccc8)ccc7o6)cc5c5cc(-c6nc7cc(-c8ccccc8)ccc7o6)ccc5c4o3)cc2)cc1]

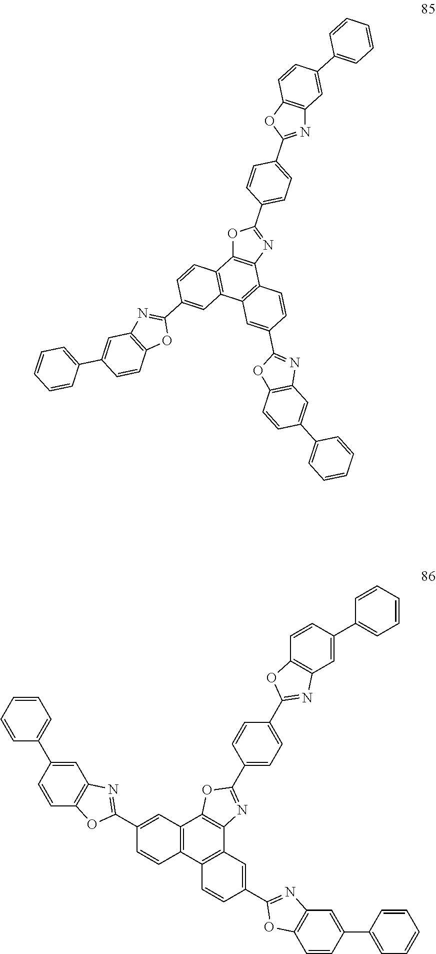 [SMILES: c1ccc(-c2ccc3oc(-c4ccc(-c5nc6c7ccc(-c8nc9cc(-c%10ccccc%10)ccc9o8)cc7c7cc(-c8nc9cc(-c%10ccccc%10)ccc9o8)ccc7c6o5)cc4)nc3c2)cc1 ; c1ccc(-c2ccc3oc(-c4ccc(-c5nc6c7cc(-c8nc9cc(-c%10ccccc%10)ccc9o8)ccc7c7ccc(-c8nc9cc(-c%10ccccc%10)ccc9o8)cc7c6o5)cc4)nc3c2)cc1]

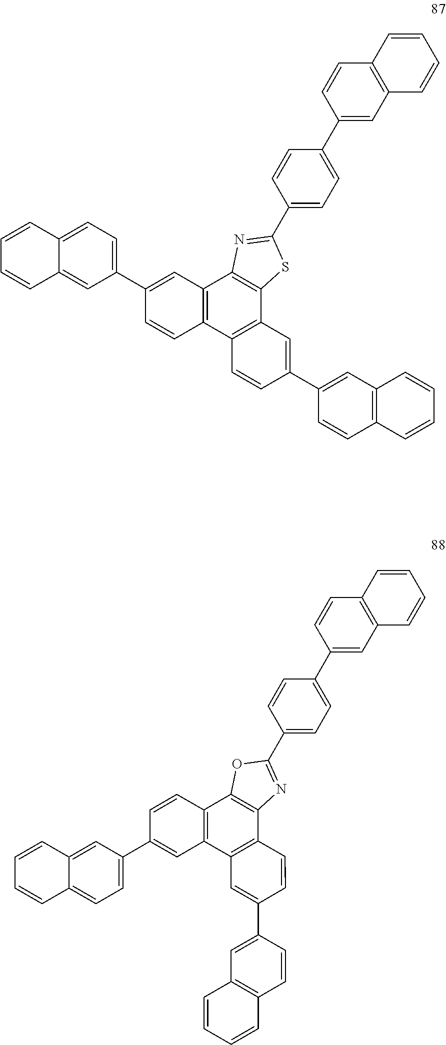 [SMILES: c1ccc2cc(-c3ccc(-c4nc5c6cc(-c7ccc8ccccc8c7)ccc6c6ccc(-c7ccc8ccccc8c7)cc6c5s4)cc3)ccc2c1 ; c1ccc2cc(-c3ccc(-c4nc5c6ccc(-c7ccc8ccccc8c7)cc6c6cc(-c7ccc8ccccc8c7)ccc6c5o4)cc3)ccc2c1]

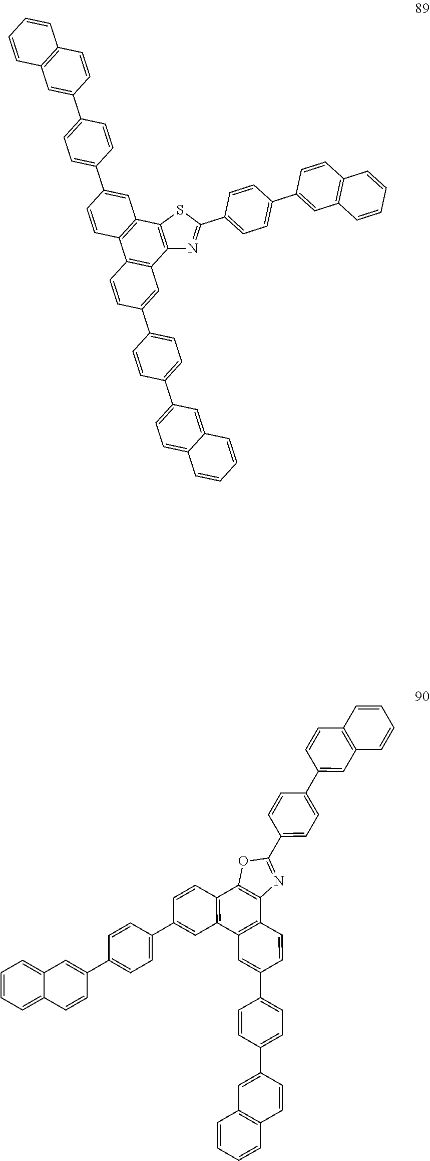 [SMILES: c1ccc2cc(-c3ccc(-c4ccc5c6ccc(-c7ccc(-c8ccc9ccccc9c8)cc7)cc6c6sc(-c7ccc(-c8ccc9ccccc9c8)cc7)nc6c5c4)cc3)ccc2c1 ; c1ccc2cc(-c3ccc(-c4ccc5c(c4)c4cc(-c6ccc(-c7ccc8ccccc8c7)cc6)ccc4c4oc(-c6ccc(-c7ccc8ccccc8c7)cc6)nc54)cc3)ccc2c1]

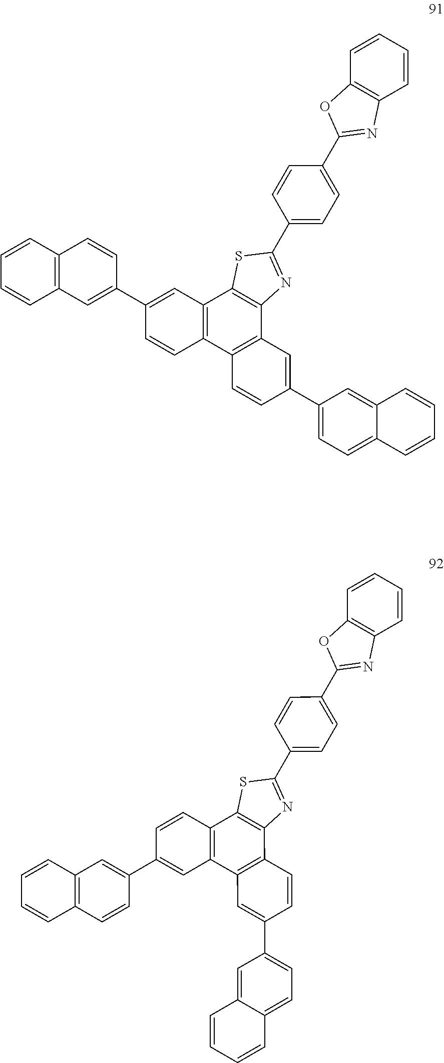 [SMILES: c1ccc2cc(-c3ccc4c5ccc(-c6ccc7ccccc7c6)cc5c5sc(-c6ccc(-c7nc8ccccc8o7)cc6)nc5c4c3)ccc2c1 ; c1ccc2cc(-c3ccc4c(c3)c3cc(-c5ccc6ccccc6c5)ccc3c3sc(-c5ccc(-c6nc7ccccc7o6)cc5)nc43)ccc2c1]

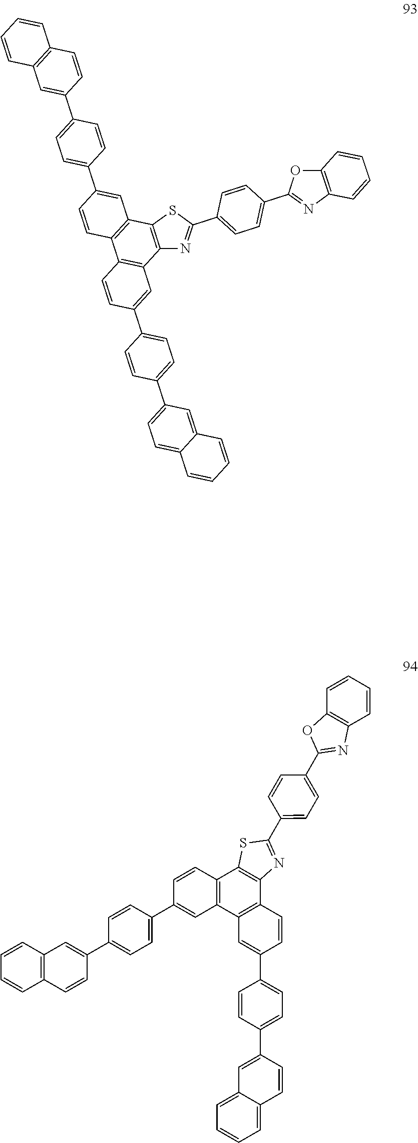 [SMILES: c1ccc2cc(-c3ccc(-c4ccc5c6ccc(-c7ccc(-c8ccc9ccccc9c8)cc7)cc6c6sc(-c7ccc(-c8nc9ccccc9o8)cc7)nc6c5c4)cc3)ccc2c1 ; c1ccc2cc(-c3ccc(-c4ccc5c(c4)c4cc(-c6ccc(-c7ccc8ccccc8c7)cc6)ccc4c4sc(-c6ccc(-c7nc8ccccc8o7)cc6)nc54)cc3)ccc2c1]

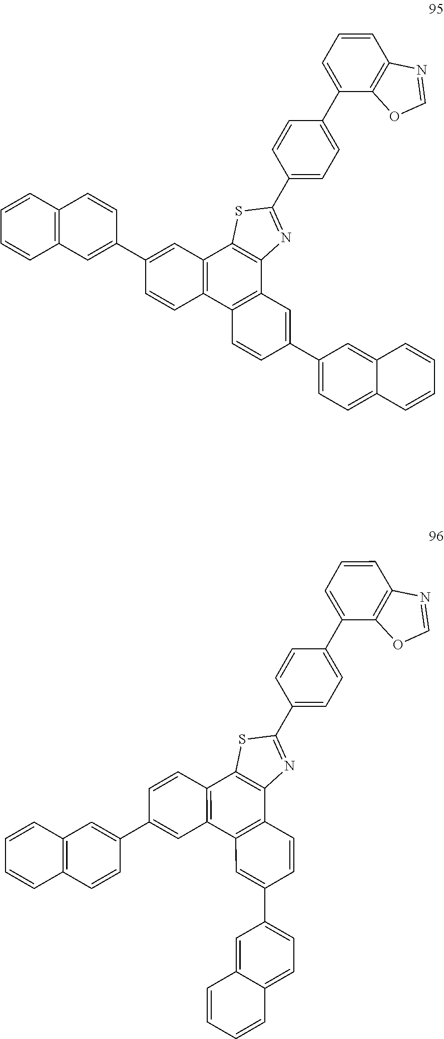 [SMILES: c1ccc2cc(-c3ccc4c5ccc(-c6ccc7ccccc7c6)cc5c5sc(-c6ccc(-c7cccc8ncoc78)cc6)nc5c4c3)ccc2c1 ; c1ccc2cc(-c3ccc4c(c3)c3cc(-c5ccc6ccccc6c5)ccc3c3sc(-c5ccc(-c6cccc7ncoc67)cc5)nc43)ccc2c1]

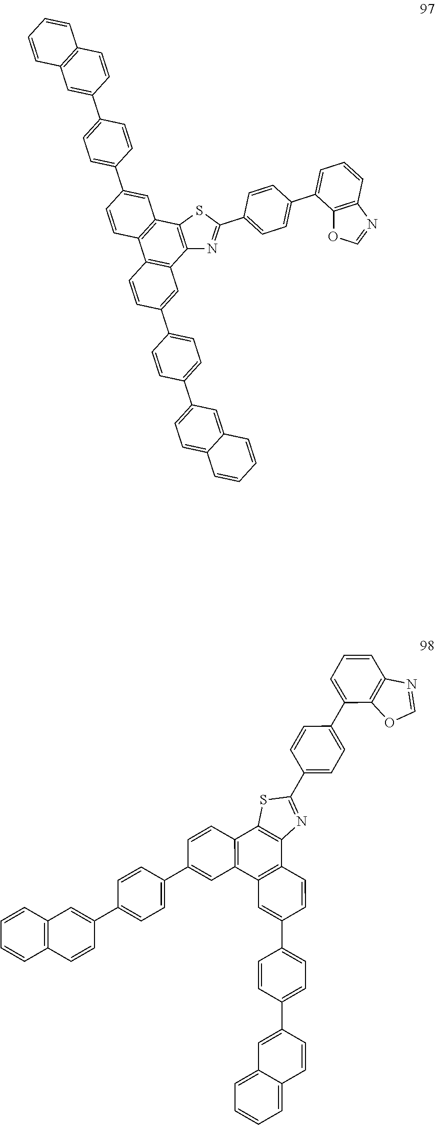 [SMILES: c1ccc2cc(-c3ccc(-c4ccc5c6ccc(-c7ccc(-c8ccc9ccccc9c8)cc7)cc6c6sc(-c7ccc(-c8cccc9ncoc89)cc7)nc6c5c4)cc3)ccc2c1 ; c1ccc2cc(-c3ccc(-c4ccc5c(c4)c4cc(-c6ccc(-c7ccc8ccccc8c7)cc6)ccc4c4sc(-c6ccc(-c7cccc8ncoc78)cc6)nc54)cc3)ccc2c1]

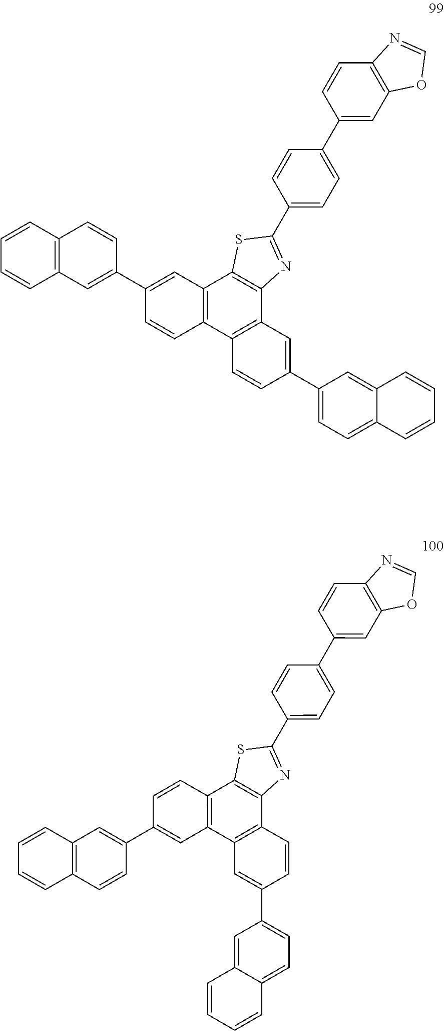 [SMILES: c1ccc2cc(-c3ccc4c5ccc(-c6ccc7ccccc7c6)cc5c5sc(-c6ccc(-c7ccc8ncoc8c7)cc6)nc5c4c3)ccc2c1 ; c1ccc2cc(-c3ccc4c(c3)c3cc(-c5ccc6ccccc6c5)ccc3c3sc(-c5ccc(-c6ccc7ncoc7c6)cc5)nc43)ccc2c1]

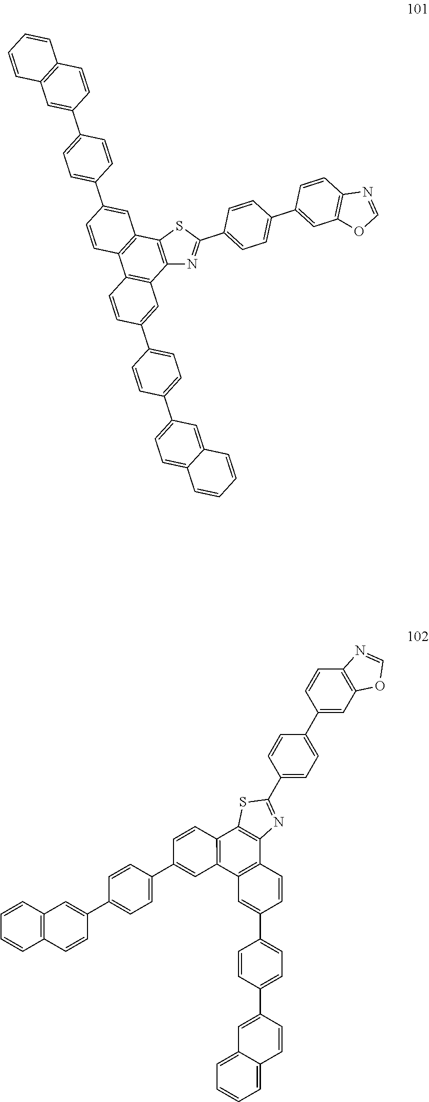 [SMILES: c1ccc2cc(-c3ccc(-c4ccc5c6ccc(-c7ccc(-c8ccc9ccccc9c8)cc7)cc6c6sc(-c7ccc(-c8ccc9ncoc9c8)cc7)nc6c5c4)cc3)ccc2c1 ; c1ccc2cc(-c3ccc(-c4ccc5c(c4)c4cc(-c6ccc(-c7ccc8ccccc8c7)cc6)ccc4c4sc(-c6ccc(-c7ccc8ncoc8c7)cc6)nc54)cc3)ccc2c1]

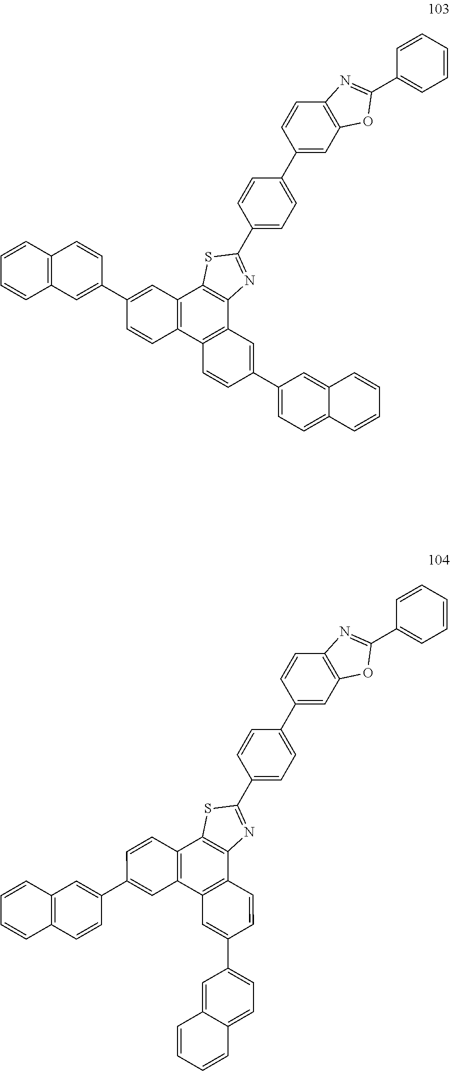 [SMILES: c1ccc(-c2nc3ccc(-c4ccc(-c5nc6c7cc(-c8ccc9ccccc9c8)ccc7c7ccc(-c8ccc9ccccc9c8)cc7c6s5)cc4)cc3o2)cc1 ; c1ccc(-c2nc3ccc(-c4ccc(-c5nc6c7ccc(-c8ccc9ccccc9c8)cc7c7cc(-c8ccc9ccccc9c8)ccc7c6s5)cc4)cc3o2)cc1]

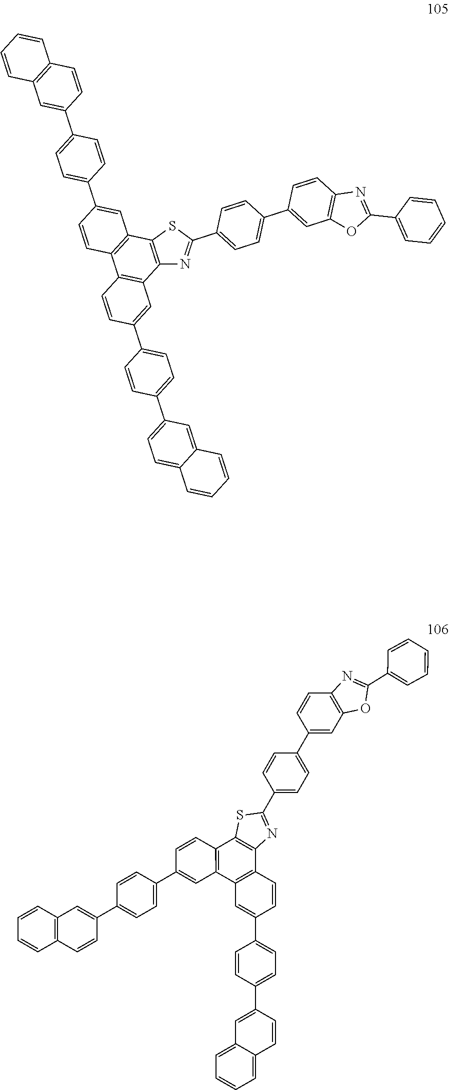 [SMILES: c1ccc(-c2nc3ccc(-c4ccc(-c5nc6c7cc(-c8ccc(-c9ccc%10ccccc%10c9)cc8)ccc7c7ccc(-c8ccc(-c9ccc%10ccccc%10c9)cc8)cc7c6s5)cc4)cc3o2)cc1 ; c1ccc(-c2nc3ccc(-c4ccc(-c5nc6c7ccc(-c8ccc(-c9ccc%10ccccc%10c9)cc8)cc7c7cc(-c8ccc(-c9ccc%10ccccc%10c9)cc8)ccc7c6s5)cc4)cc3o2)cc1]

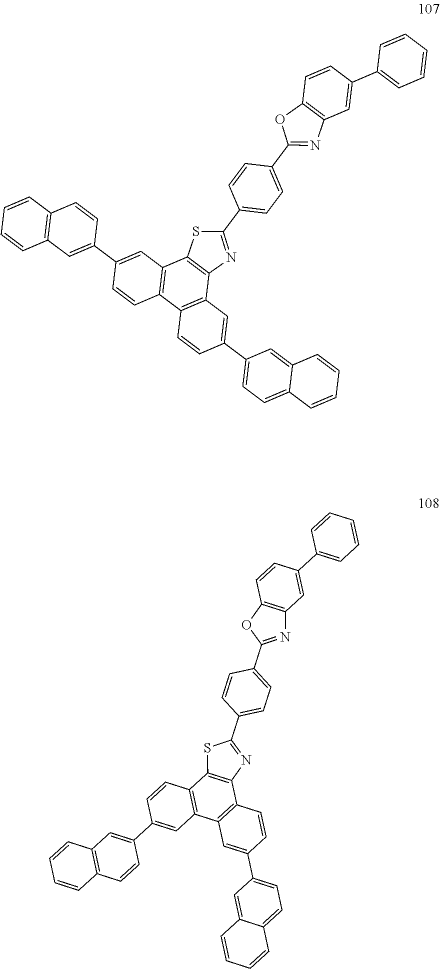 [SMILES: c1ccc(-c2ccc3oc(-c4ccc(-c5nc6c7cc(-c8ccc9ccccc9c8)ccc7c7ccc(-c8ccc9ccccc9c8)cc7c6s5)cc4)nc3c2)cc1 ; c1ccc(-c2ccc3oc(-c4ccc(-c5nc6c7ccc(-c8ccc9ccccc9c8)cc7c7cc(-c8ccc9ccccc9c8)ccc7c6s5)cc4)nc3c2)cc1]

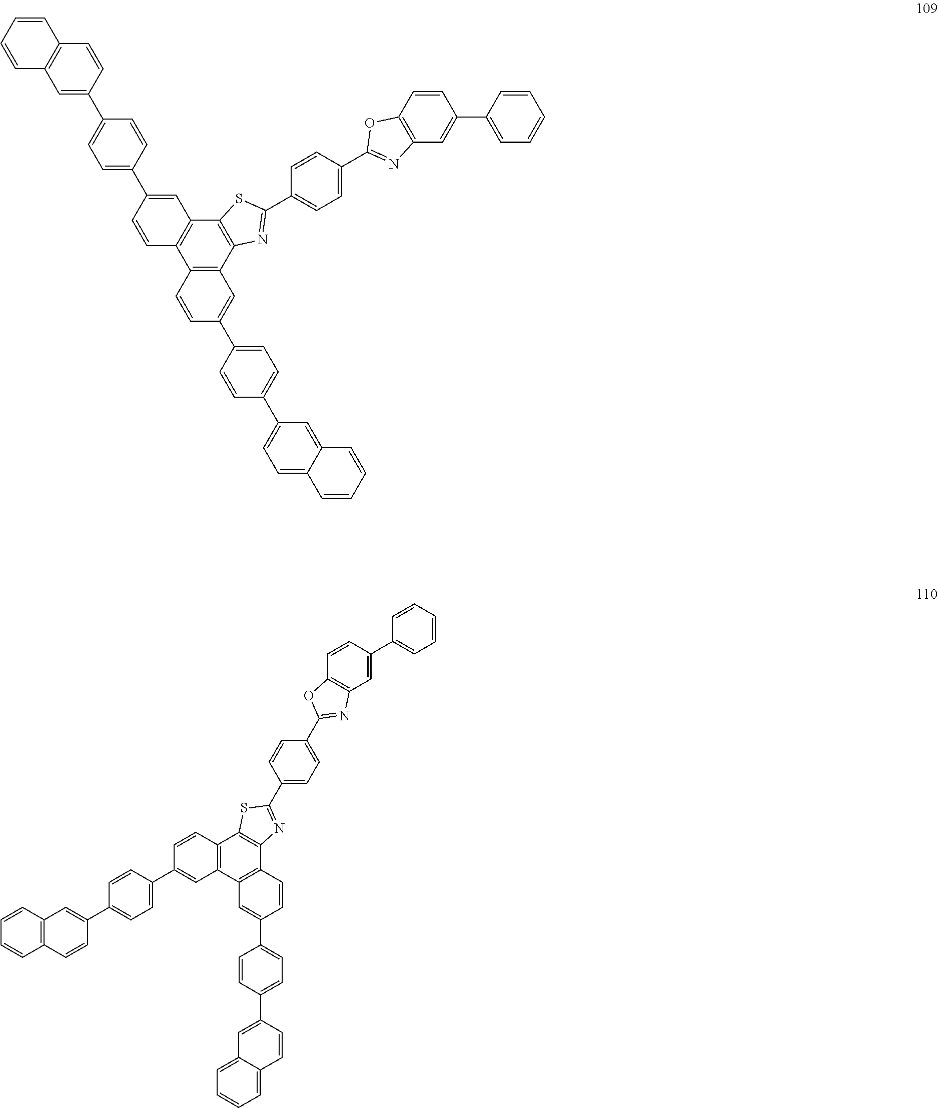 [SMILES: c1ccc(-c2ccc3oc(-c4ccc(-c5nc6c7cc(-c8ccc(-c9ccc%10ccccc%10c9)cc8)ccc7c7ccc(-c8ccc(-c9ccc%10ccccc%10c9)cc8)cc7c6s5)cc4)nc3c2)cc1 ; c1ccc(-c2ccc3oc(-c4ccc(-c5nc6c7ccc(-c8ccc(-c9ccc%10ccccc%10c9)cc8)cc7c7cc(-c8ccc(-c9ccc%10ccccc%10c9)cc8)ccc7c6s5)cc4)nc3c2)cc1]

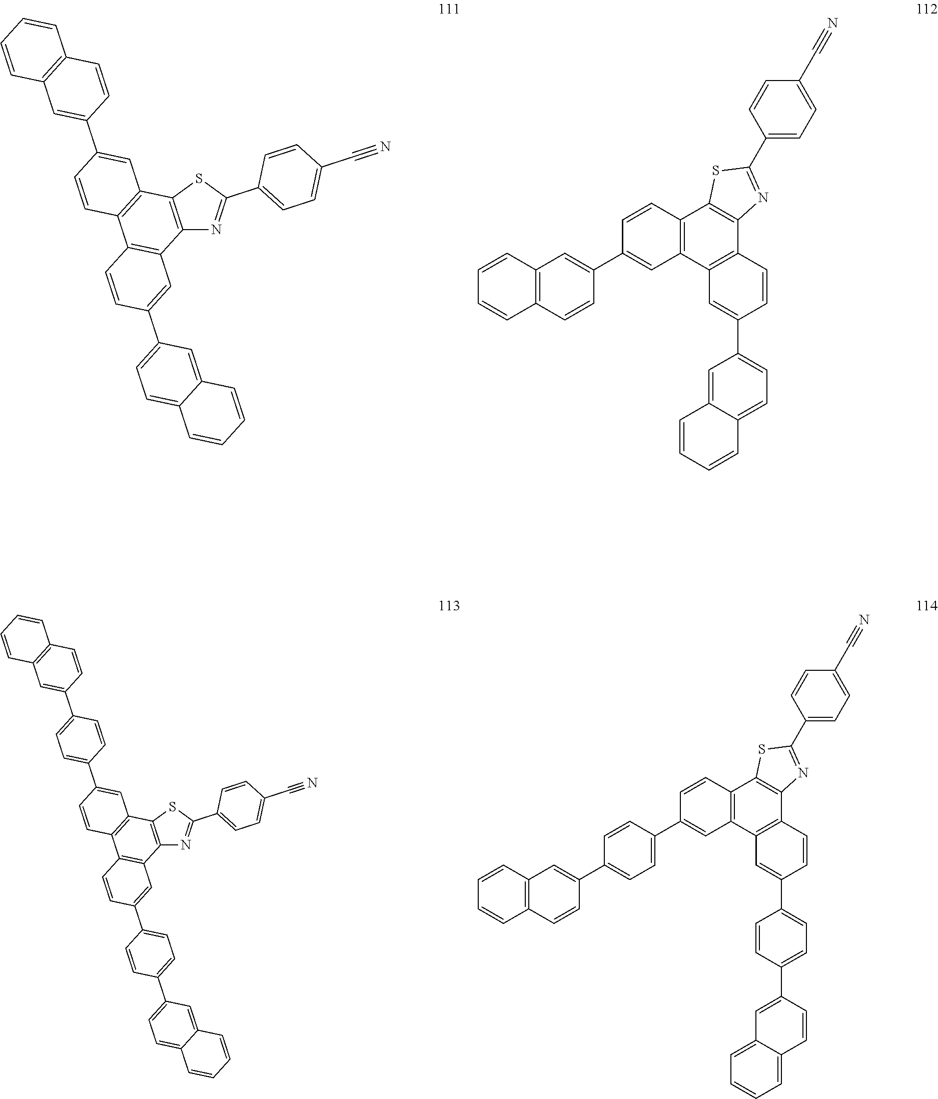 [SMILES: N#Cc1ccc(-c2nc3c4cc(-c5ccc6ccccc6c5)ccc4c4ccc(-c5ccc6ccccc6c5)cc4c3s2)cc1 ; N#Cc1ccc(-c2nc3c4ccc(-c5ccc6ccccc6c5)cc4c4cc(-c5ccc6ccccc6c5)ccc4c3s2)cc1 ; N#Cc1ccc(-c2nc3c4cc(-c5ccc(-c6ccc7ccccc7c6)cc5)ccc4c4ccc(-c5ccc(-c6ccc7ccccc7c6)cc5)cc4c3s2)cc1 ; N#Cc1ccc(-c2nc3c4ccc(-c5ccc(-c6ccc7ccccc7c6)cc5)cc4c4cc(-c5ccc(-c6ccc7ccccc7c6)cc5)ccc4c3s2)cc1]

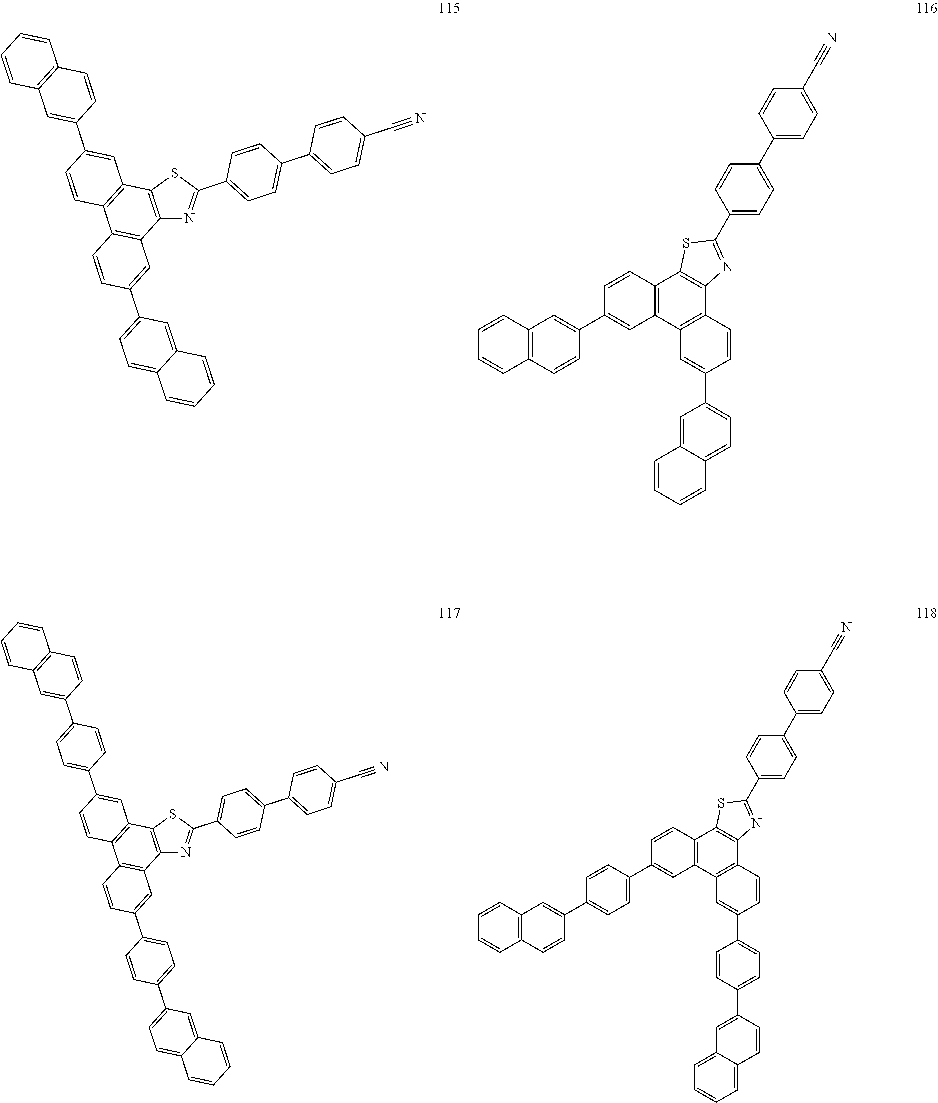 [SMILES: N#Cc1ccc(-c2ccc(-c3nc4c5cc(-c6ccc7ccccc7c6)ccc5c5ccc(-c6ccc7ccccc7c6)cc5c4s3)cc2)cc1 ; N#Cc1ccc(-c2ccc(-c3nc4c5ccc(-c6ccc7ccccc7c6)cc5c5cc(-c6ccc7ccccc7c6)ccc5c4s3)cc2)cc1 ; N#Cc1ccc(-c2ccc(-c3nc4c5cc(-c6ccc(-c7ccc8ccccc8c7)cc6)ccc5c5ccc(-c6ccc(-c7ccc8ccccc8c7)cc6)cc5c4s3)cc2)cc1 ; N#Cc1ccc(-c2ccc(-c3nc4c5ccc(-c6ccc(-c7ccc8ccccc8c7)cc6)cc5c5cc(-c6ccc(-c7ccc8ccccc8c7)cc6)ccc5c4s3)cc2)cc1]

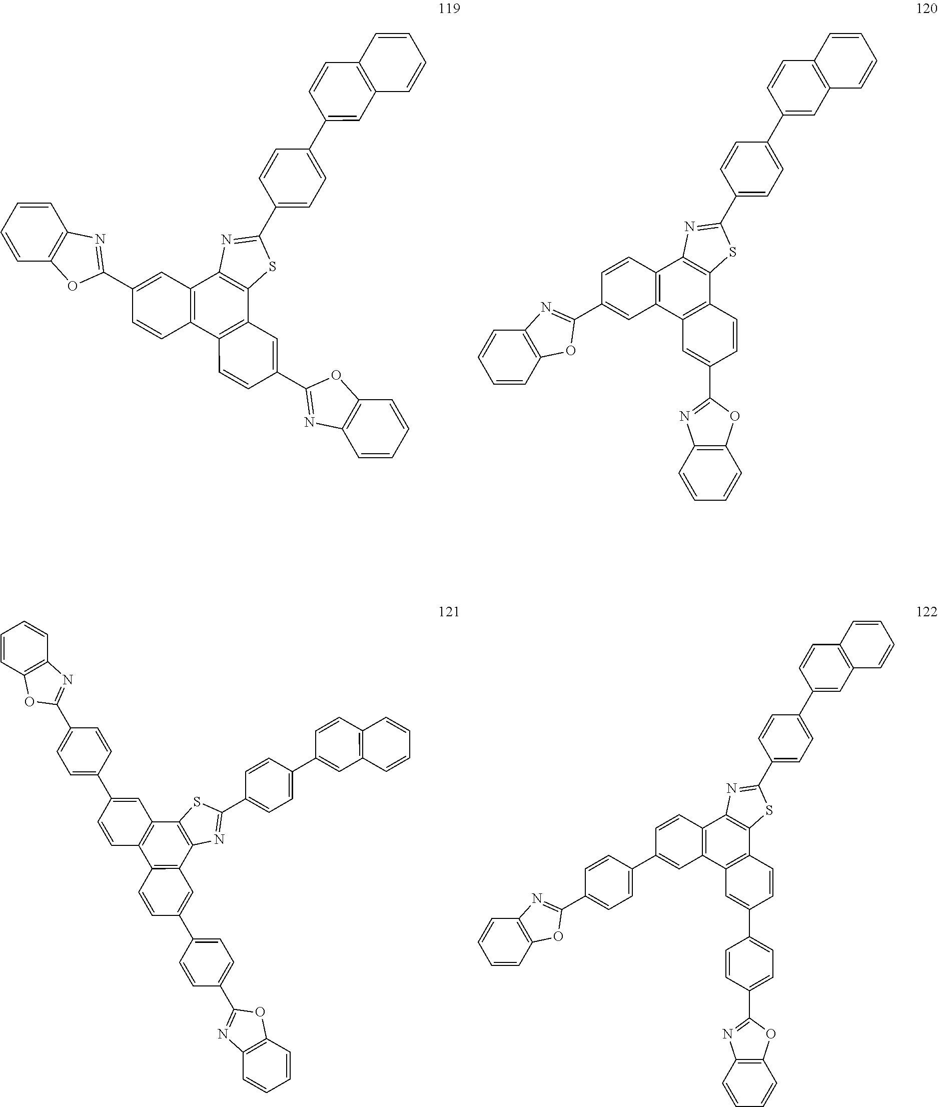 [SMILES: c1ccc2cc(-c3ccc(-c4nc5c6cc(-c7nc8ccccc8o7)ccc6c6ccc(-c7nc8ccccc8o7)cc6c5s4)cc3)ccc2c1 ; c1ccc2cc(-c3ccc(-c4nc5c6ccc(-c7nc8ccccc8o7)cc6c6cc(-c7nc8ccccc8o7)ccc6c5s4)cc3)ccc2c1 ; c1ccc2cc(-c3ccc(-c4nc5c6cc(-c7ccc(-c8nc9ccccc9o8)cc7)ccc6c6ccc(-c7ccc(-c8nc9ccccc9o8)cc7)cc6c5s4)cc3)ccc2c1 ; c1ccc2cc(-c3ccc(-c4nc5c6ccc(-c7ccc(-c8nc9ccccc9o8)cc7)cc6c6cc(-c7ccc(-c8nc9ccccc9o8)cc7)ccc6c5s4)cc3)ccc2c1]

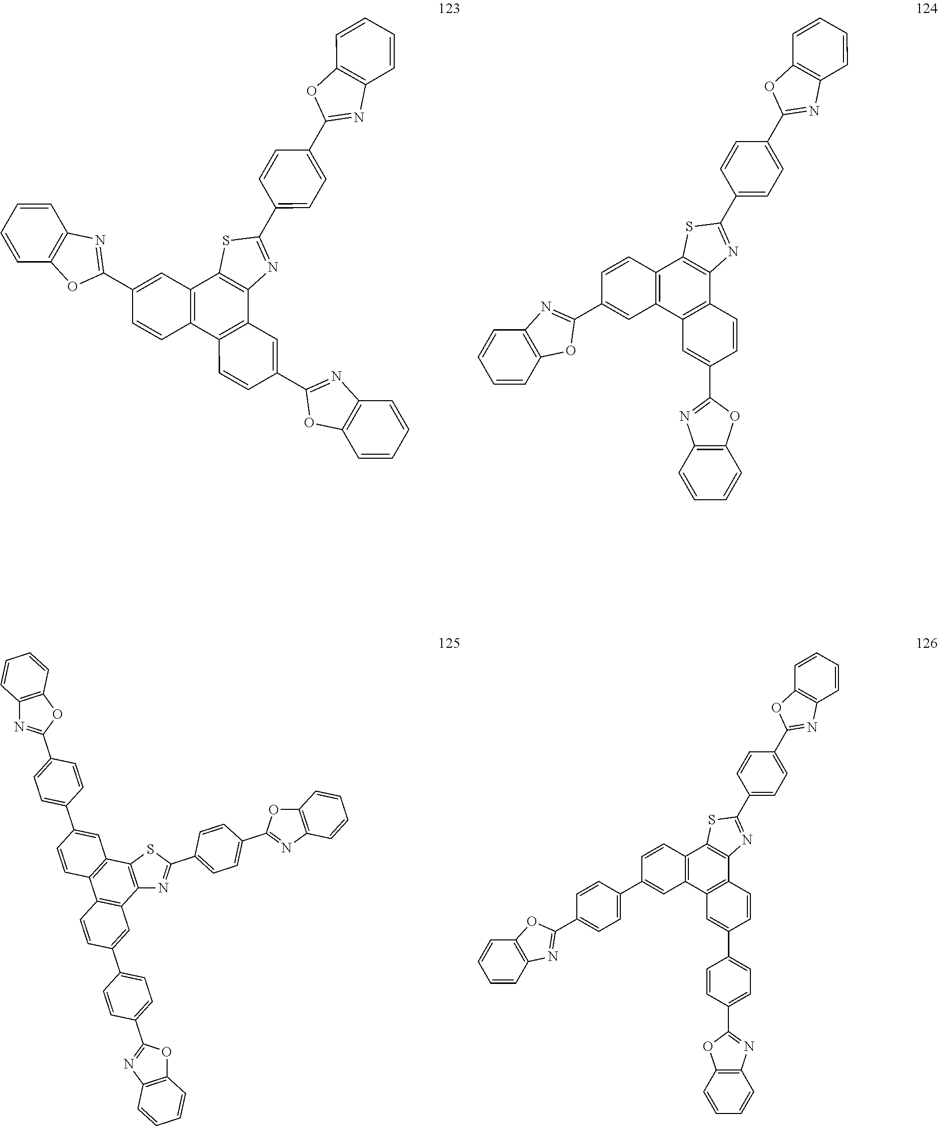 [SMILES: c1ccc2oc(-c3ccc(-c4nc5c6cc(-c7nc8ccccc8o7)ccc6c6ccc(-c7nc8ccccc8o7)cc6c5s4)cc3)nc2c1 ; c1ccc2oc(-c3ccc(-c4nc5c6ccc(-c7nc8ccccc8o7)cc6c6cc(-c7nc8ccccc8o7)ccc6c5s4)cc3)nc2c1 ; c1ccc2oc(-c3ccc(-c4ccc5c6ccc(-c7ccc(-c8nc9ccccc9o8)cc7)cc6c6sc(-c7ccc(-c8nc9ccccc9o8)cc7)nc6c5c4)cc3)nc2c1 ; c1ccc2oc(-c3ccc(-c4ccc5c(c4)c4cc(-c6ccc(-c7nc8ccccc8o7)cc6)ccc4c4sc(-c6ccc(-c7nc8ccccc8o7)cc6)nc54)cc3)nc2c1]

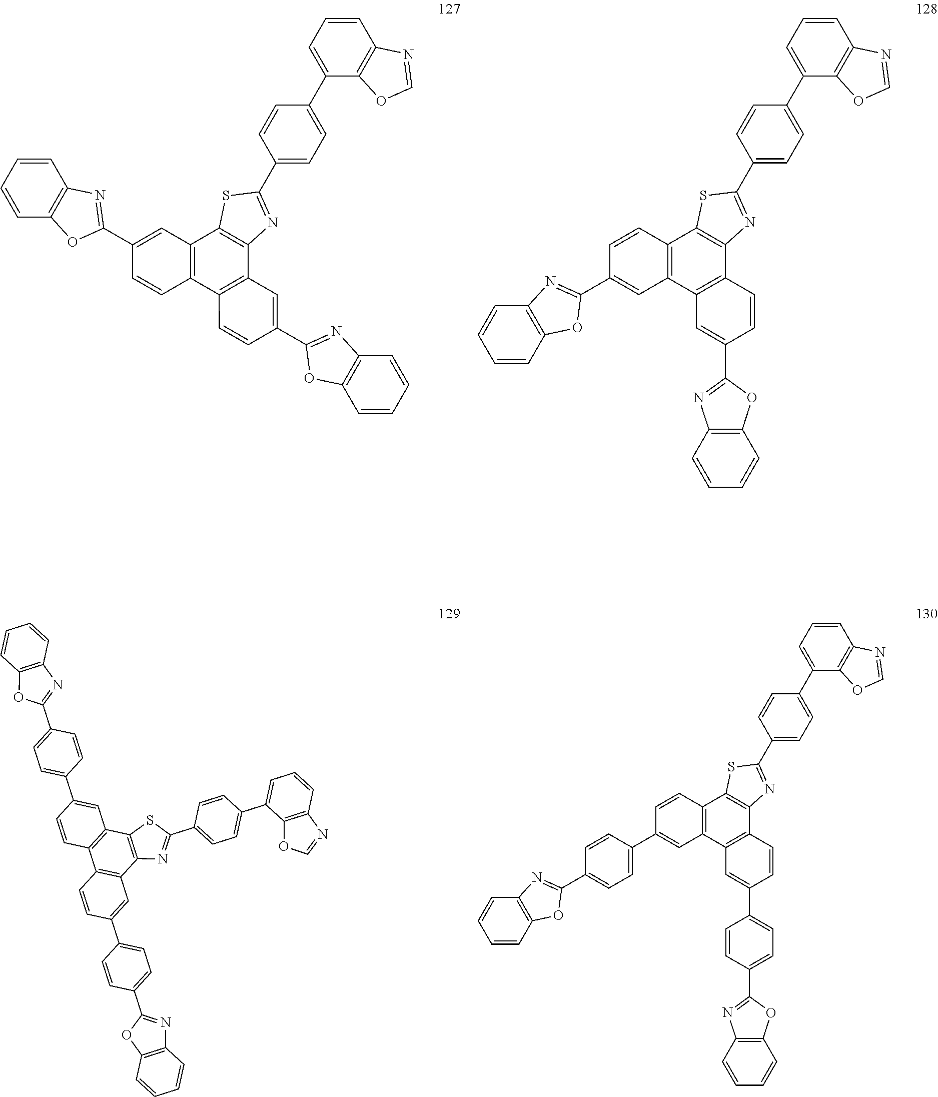 [SMILES: c1ccc2oc(-c3ccc4c5ccc(-c6nc7ccccc7o6)cc5c5sc(-c6ccc(-c7cccc8ncoc78)cc6)nc5c4c3)nc2c1 ; c1ccc2oc(-c3ccc4c(c3)c3cc(-c5nc6ccccc6o5)ccc3c3sc(-c5ccc(-c6cccc7ncoc67)cc5)nc43)nc2c1 ; c1ccc2oc(-c3ccc(-c4ccc5c6ccc(-c7ccc(-c8nc9ccccc9o8)cc7)cc6c6sc(-c7ccc(-c8cccc9ncoc89)cc7)nc6c5c4)cc3)nc2c1 ; c1ccc2oc(-c3ccc(-c4ccc5c(c4)c4cc(-c6ccc(-c7nc8ccccc8o7)cc6)ccc4c4sc(-c6ccc(-c7cccc8ncoc78)cc6)nc54)cc3)nc2c1]

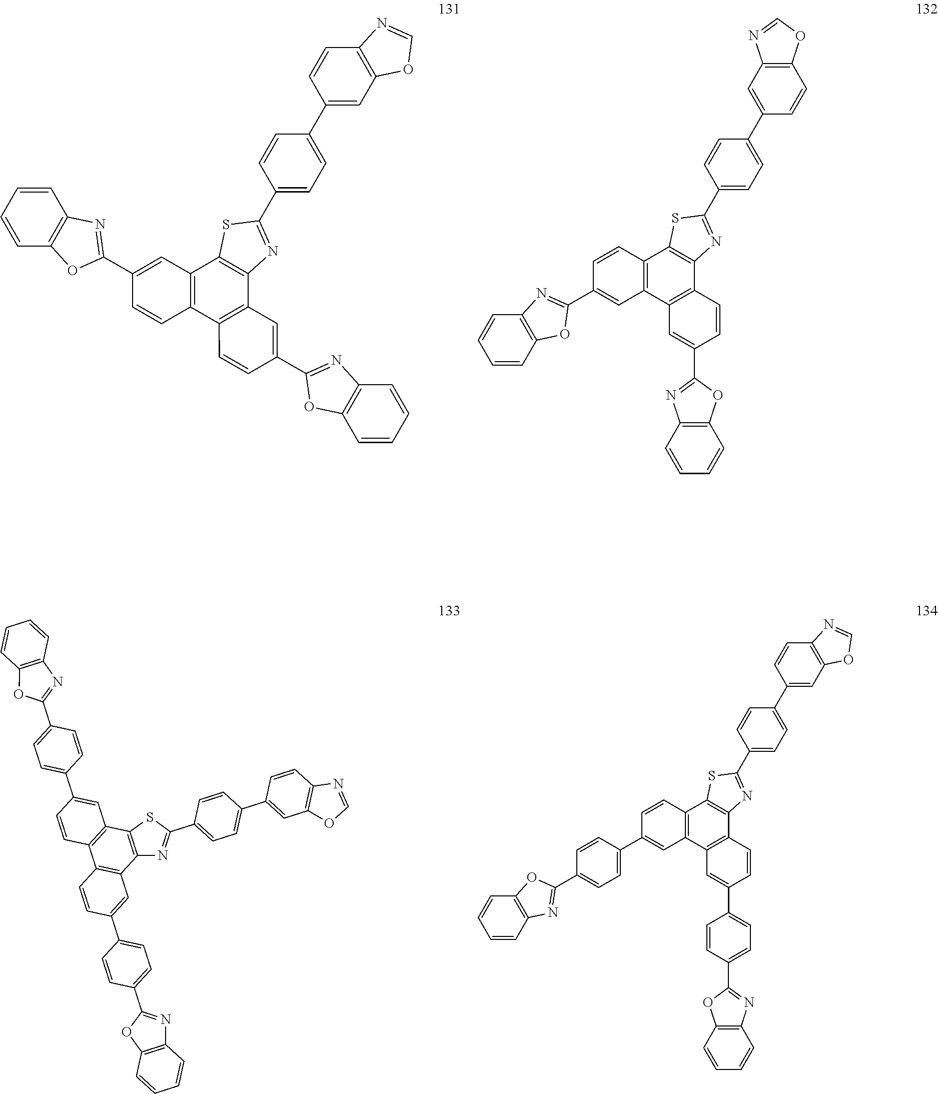 [SMILES: c1ccc2oc(-c3ccc4c5ccc(-c6nc7ccccc7o6)cc5c5sc(-c6ccc(-c7ccc8ncoc8c7)cc6)nc5c4c3)nc2c1 ; c1ccc2oc(-c3ccc4c(c3)c3cc(-c5nc6ccccc6o5)ccc3c3sc(-c5ccc(-c6ccc7ocnc7c6)cc5)nc43)nc2c1 ; c1ccc2oc(-c3ccc(-c4ccc5c6ccc(-c7ccc(-c8nc9ccccc9o8)cc7)cc6c6sc(-c7ccc(-c8ccc9ncoc9c8)cc7)nc6c5c4)cc3)nc2c1 ; c1ccc2oc(-c3ccc(-c4ccc5c(c4)c4cc(-c6ccc(-c7nc8ccccc8o7)cc6)ccc4c4sc(-c6ccc(-c7ccc8ncoc8c7)cc6)nc54)cc3)nc2c1]

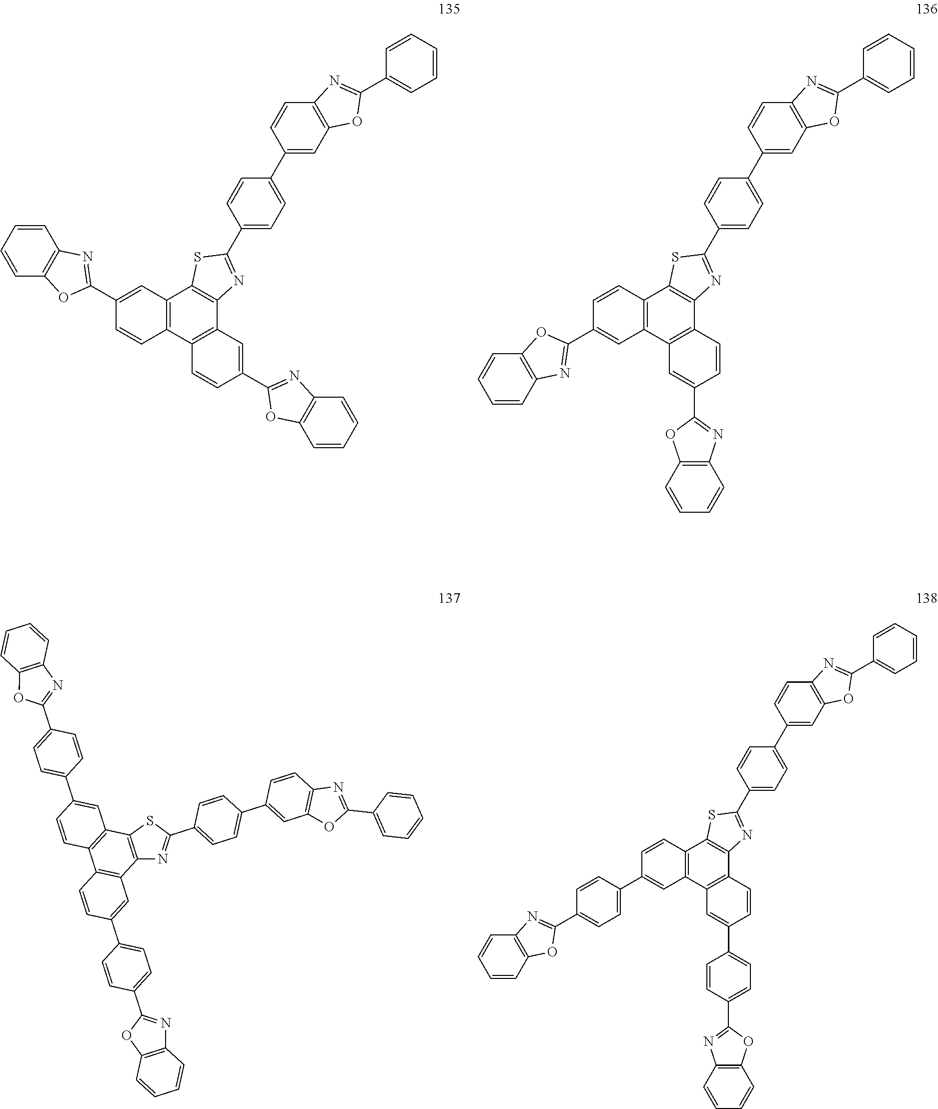 [SMILES: c1ccc(-c2nc3ccc(-c4ccc(-c5nc6c7cc(-c8nc9ccccc9o8)ccc7c7ccc(-c8nc9ccccc9o8)cc7c6s5)cc4)cc3o2)cc1 ; c1ccc(-c2nc3ccc(-c4ccc(-c5nc6c7ccc(-c8nc9ccccc9o8)cc7c7cc(-c8nc9ccccc9o8)ccc7c6s5)cc4)cc3o2)cc1 ; c1ccc(-c2nc3ccc(-c4ccc(-c5nc6c7cc(-c8ccc(-c9nc%10ccccc%10o9)cc8)ccc7c7ccc(-c8ccc(-c9nc%10ccccc%10o9)cc8)cc7c6s5)cc4)cc3o2)cc1 ; c1ccc(-c2nc3ccc(-c4ccc(-c5nc6c7ccc(-c8ccc(-c9nc%10ccccc%10o9)cc8)cc7c7cc(-c8ccc(-c9nc%10ccccc%10o9)cc8)ccc7c6s5)cc4)cc3o2)cc1]

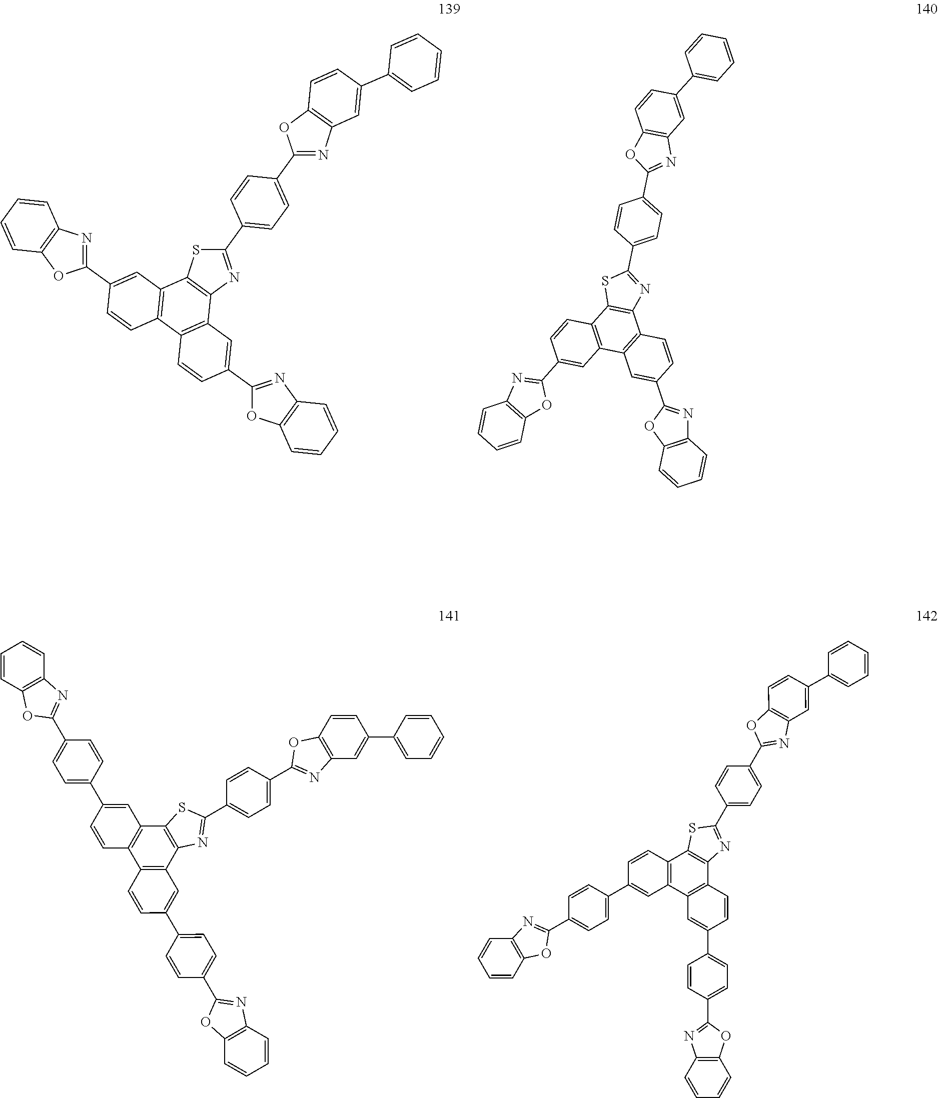 [SMILES: c1ccc(-c2ccc3oc(-c4ccc(-c5nc6c7cc(-c8nc9ccccc9o8)ccc7c7ccc(-c8nc9ccccc9o8)cc7c6s5)cc4)nc3c2)cc1 ; c1ccc(-c2ccc3oc(-c4ccc(-c5nc6c7ccc(-c8nc9ccccc9o8)cc7c7cc(-c8nc9ccccc9o8)ccc7c6s5)cc4)nc3c2)cc1 ; c1ccc(-c2ccc3oc(-c4ccc(-c5nc6c7cc(-c8ccc(-c9nc%10ccccc%10o9)cc8)ccc7c7ccc(-c8ccc(-c9nc%10ccccc%10o9)cc8)cc7c6s5)cc4)nc3c2)cc1 ; c1ccc(-c2ccc3oc(-c4ccc(-c5nc6c7ccc(-c8ccc(-c9nc%10ccccc%10o9)cc8)cc7c7cc(-c8ccc(-c9nc%10ccccc%10o9)cc8)ccc7c6s5)cc4)nc3c2)cc1]

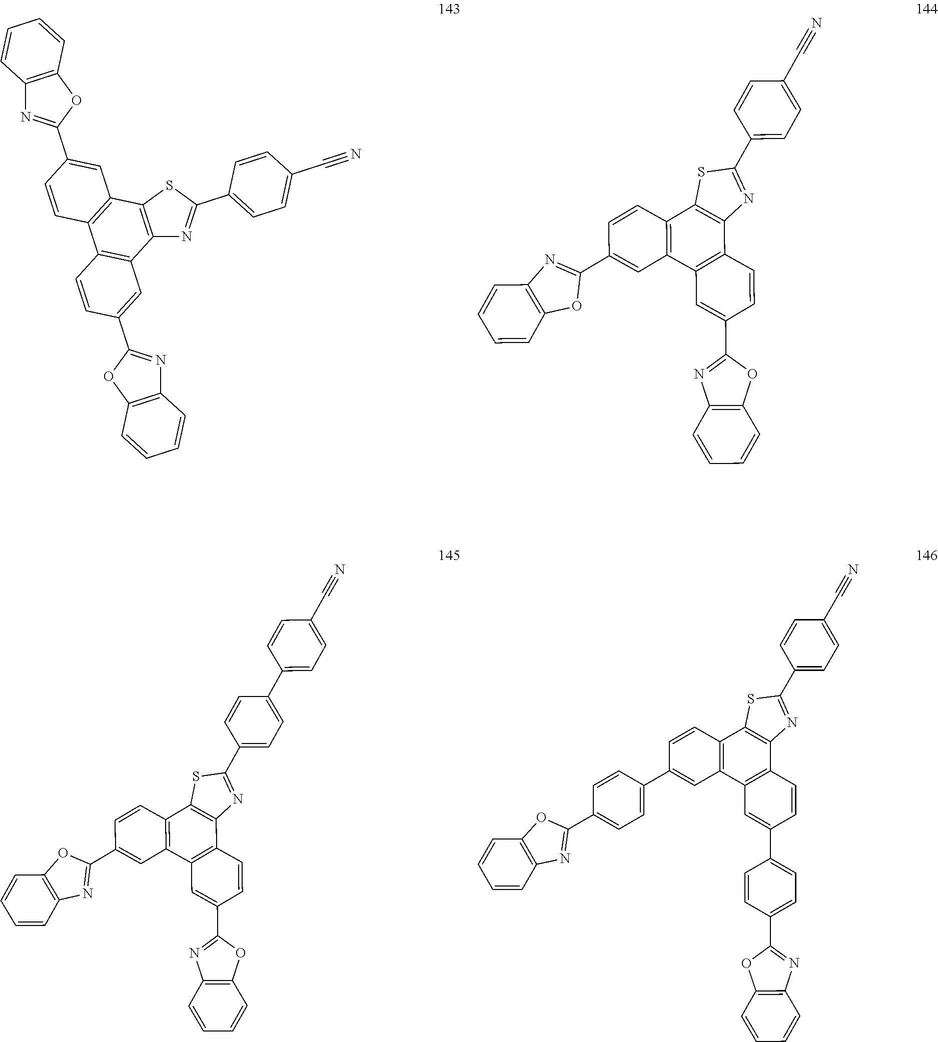 [SMILES: N#Cc1ccc(-c2nc3c4cc(-c5nc6ccccc6o5)ccc4c4ccc(-c5nc6ccccc6o5)cc4c3s2)cc1 ; N#Cc1ccc(-c2nc3c4ccc(-c5nc6ccccc6o5)cc4c4cc(-c5nc6ccccc6o5)ccc4c3s2)cc1 ; N#Cc1ccc(-c2ccc(-c3nc4c5ccc(-c6nc7ccccc7o6)cc5c5cc(-c6nc7ccccc7o6)ccc5c4s3)cc2)cc1 ; N#Cc1ccc(-c2nc3c4ccc(-c5ccc(-c6nc7ccccc7o6)cc5)cc4c4cc(-c5ccc(-c6nc7ccccc7o6)cc5)ccc4c3s2)cc1]

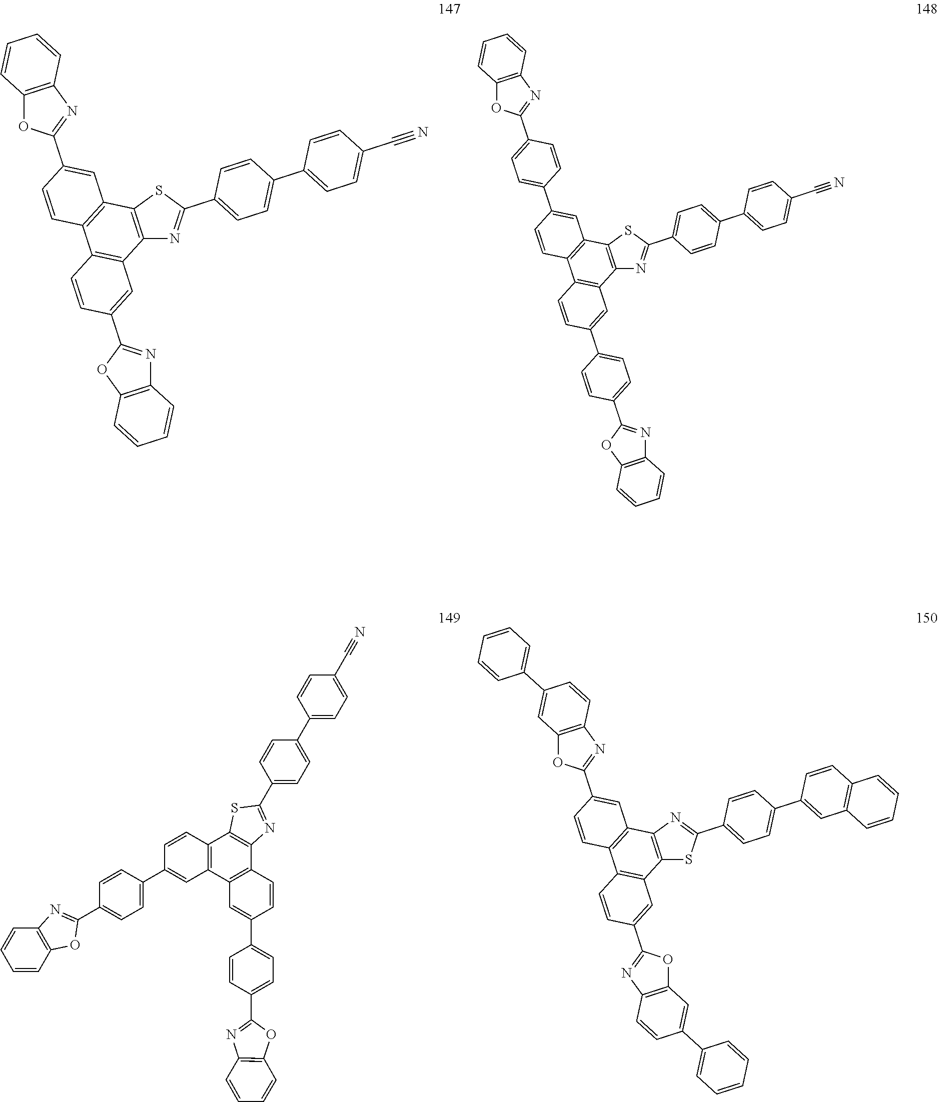 [SMILES: N#Cc1ccc(-c2ccc(-c3nc4c5cc(-c6nc7ccccc7o6)ccc5c5ccc(-c6nc7ccccc7o6)cc5c4s3)cc2)cc1 ; N#Cc1ccc(-c2ccc(-c3nc4c5cc(-c6ccc(-c7nc8ccccc8o7)cc6)ccc5c5ccc(-c6ccc(-c7nc8ccccc8o7)cc6)cc5c4s3)cc2)cc1 ; N#Cc1ccc(-c2ccc(-c3nc4c5ccc(-c6ccc(-c7nc8ccccc8o7)cc6)cc5c5cc(-c6ccc(-c7nc8ccccc8o7)cc6)ccc5c4s3)cc2)cc1 ; c1ccc(-c2ccc3nc(-c4ccc5c6ccc(-c7nc8ccc(-c9ccccc9)cc8o7)cc6c6sc(-c7ccc(-c8ccc9ccccc9c8)cc7)nc6c5c4)oc3c2)cc1]

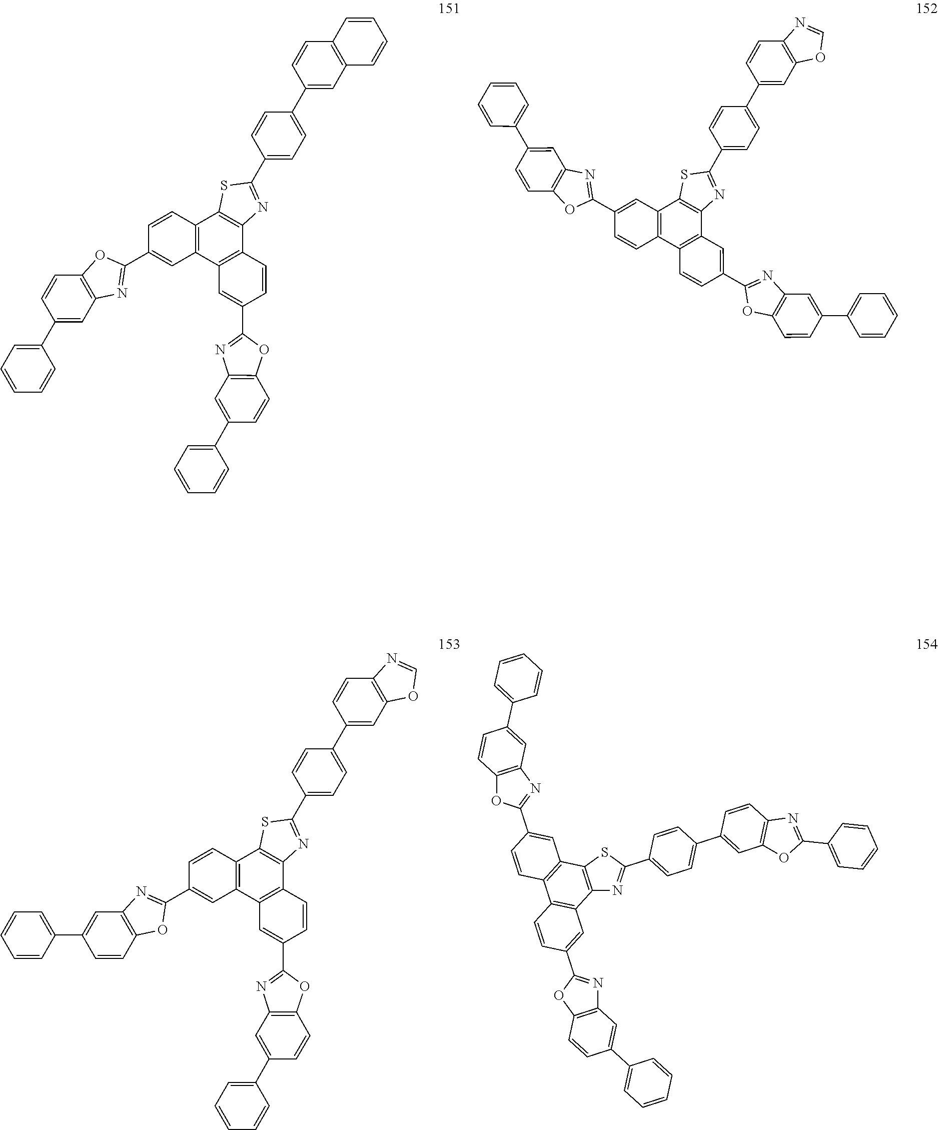 [SMILES: c1ccc(-c2ccc3oc(-c4ccc5c(c4)c4cc(-c6nc7cc(-c8ccccc8)ccc7o6)ccc4c4sc(-c6ccc(-c7ccc8ccccc8c7)cc6)nc54)nc3c2)cc1 ; c1ccc(-c2ccc3oc(-c4ccc5c6ccc(-c7nc8cc(-c9ccccc9)ccc8o7)cc6c6sc(-c7ccc(-c8ccc9ncoc9c8)cc7)nc6c5c4)nc3c2)cc1 ; c1ccc(-c2ccc3oc(-c4ccc5c(c4)c4cc(-c6nc7cc(-c8ccccc8)ccc7o6)ccc4c4sc(-c6ccc(-c7ccc8ncoc8c7)cc6)nc54)nc3c2)cc1 ; c1ccc(-c2ccc3oc(-c4ccc5c6ccc(-c7nc8cc(-c9ccccc9)ccc8o7)cc6c6sc(-c7ccc(-c8ccc9nc(-c%10ccccc%10)oc9c8)cc7)nc6c5c4)nc3c2)cc1]

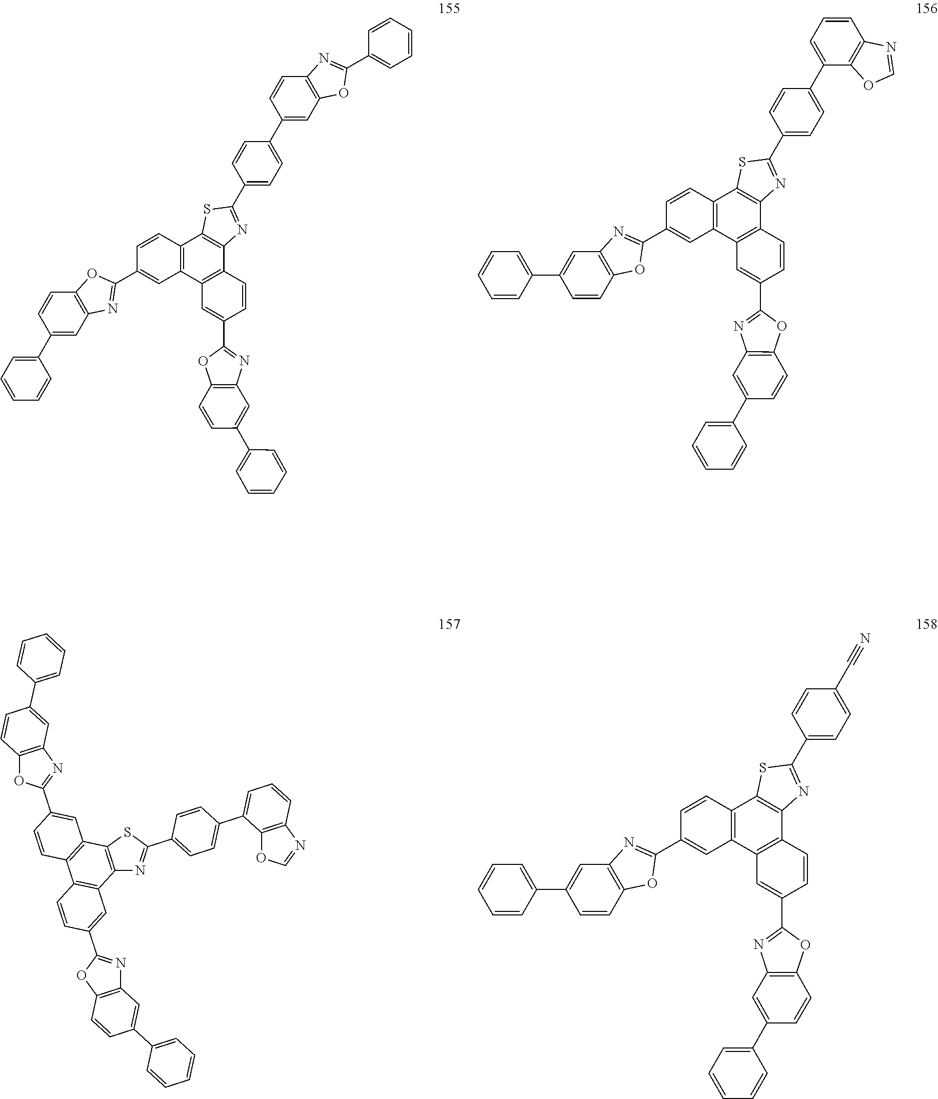 [SMILES: c1ccc(-c2ccc3oc(-c4ccc5c(c4)c4cc(-c6nc7cc(-c8ccccc8)ccc7o6)ccc4c4sc(-c6ccc(-c7ccc8nc(-c9ccccc9)oc8c7)cc6)nc54)nc3c2)cc1 ; c1ccc(-c2ccc3oc(-c4ccc5c(c4)c4cc(-c6nc7cc(-c8ccccc8)ccc7o6)ccc4c4sc(-c6ccc(-c7cccc8ncoc78)cc6)nc54)nc3c2)cc1 ; c1ccc(-c2ccc3oc(-c4ccc5c6ccc(-c7nc8cc(-c9ccccc9)ccc8o7)cc6c6sc(-c7ccc(-c8cccc9ncoc89)cc7)nc6c5c4)nc3c2)cc1 ; N#Cc1ccc(-c2nc3c4ccc(-c5nc6cc(-c7ccccc7)ccc6o5)cc4c4cc(-c5nc6cc(-c7ccccc7)ccc6o5)ccc4c3s2)cc1]

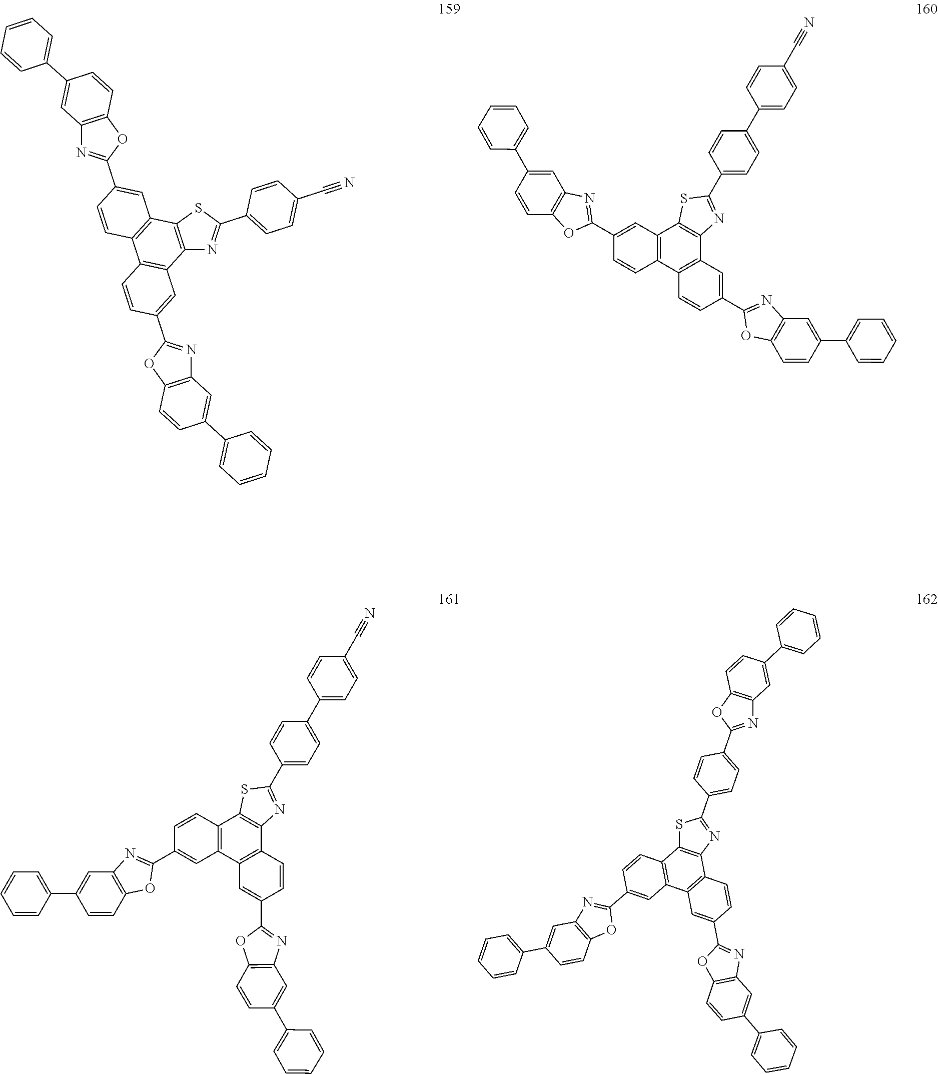 [SMILES: N#Cc1ccc(-c2nc3c4cc(-c5nc6cc(-c7ccccc7)ccc6o5)ccc4c4ccc(-c5nc6cc(-c7ccccc7)ccc6o5)cc4c3s2)cc1 ; N#Cc1ccc(-c2ccc(-c3nc4c5cc(-c6nc7cc(-c8ccccc8)ccc7o6)ccc5c5ccc(-c6nc7cc(-c8ccccc8)ccc7o6)cc5c4s3)cc2)cc1 ; N#Cc1ccc(-c2ccc(-c3nc4c5ccc(-c6nc7cc(-c8ccccc8)ccc7o6)cc5c5cc(-c6nc7cc(-c8ccccc8)ccc7o6)ccc5c4s3)cc2)cc1 ; c1ccc(-c2ccc3oc(-c4ccc(-c5nc6c7ccc(-c8nc9cc(-c%10ccccc%10)ccc9o8)cc7c7cc(-c8nc9cc(-c%10ccccc%10)ccc9o8)ccc7c6s5)cc4)nc3c2)cc1]

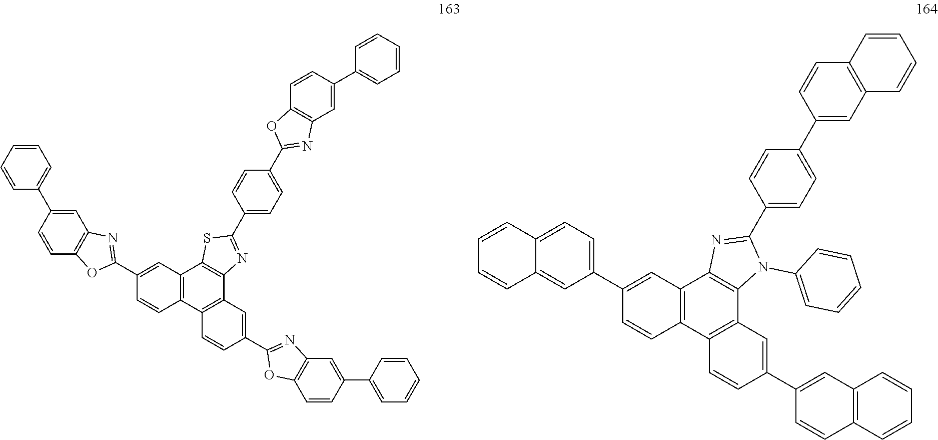 [SMILES: c1ccc(-c2ccc3oc(-c4ccc(-c5nc6c7cc(-c8nc9cc(-c%10ccccc%10)ccc9o8)ccc7c7ccc(-c8nc9cc(-c%10ccccc%10)ccc9o8)cc7c6s5)cc4)nc3c2)cc1 ; c1ccc(-n2c(-c3ccc(-c4ccc5ccccc5c4)cc3)nc3c4cc(-c5ccc6ccccc6c5)ccc4c4ccc(-c5ccc6ccccc6c5)cc4c32)cc1]

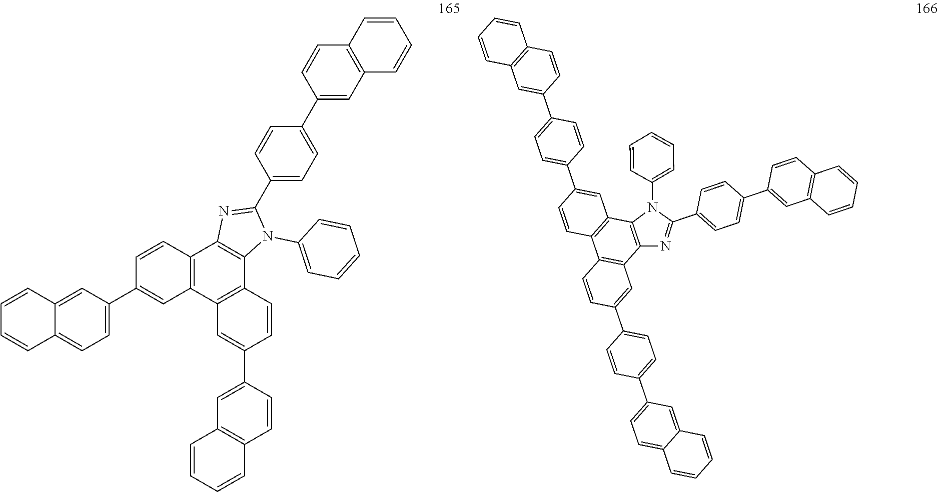 [SMILES: c1ccc(-n2c(-c3ccc(-c4ccc5ccccc5c4)cc3)nc3c4ccc(-c5ccc6ccccc6c5)cc4c4cc(-c5ccc6ccccc6c5)ccc4c32)cc1 ; c1ccc(-n2c(-c3ccc(-c4ccc5ccccc5c4)cc3)nc3c4cc(-c5ccc(-c6ccc7ccccc7c6)cc5)ccc4c4ccc(-c5ccc(-c6ccc7ccccc7c6)cc5)cc4c32)cc1]

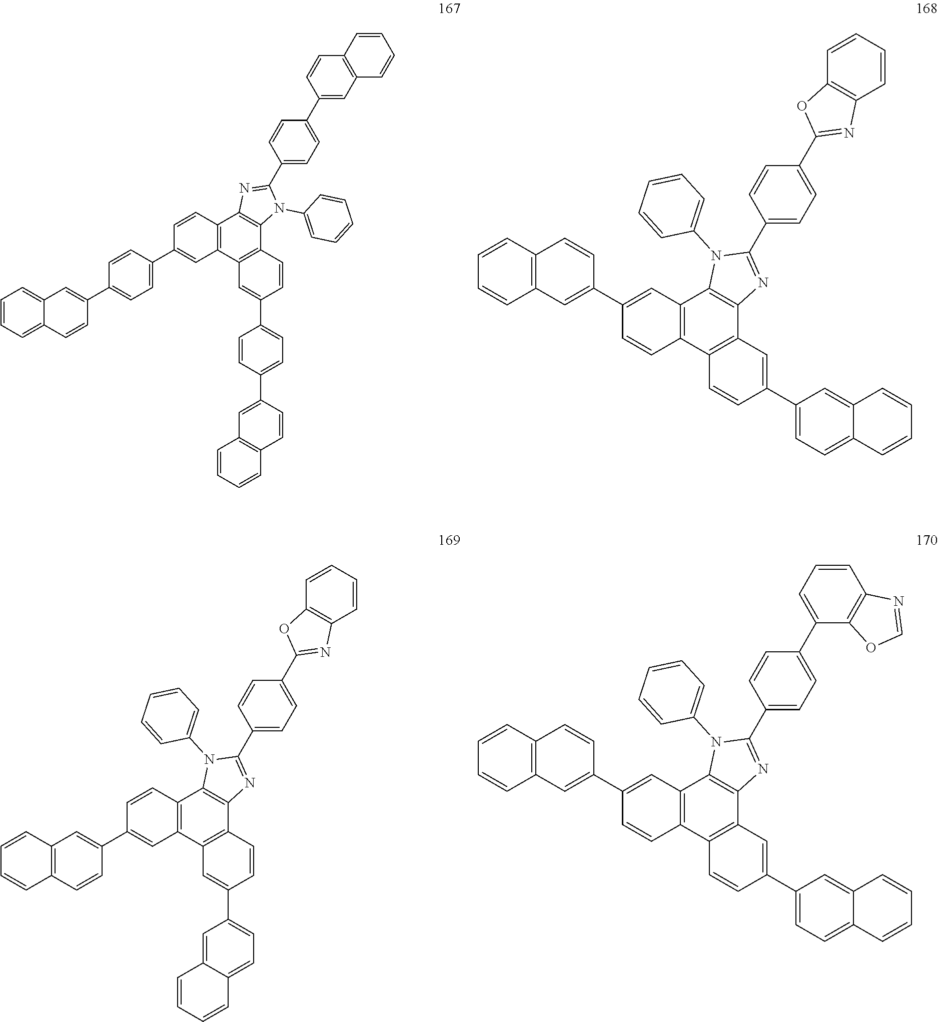 [SMILES: c1ccc(-n2c(-c3ccc(-c4ccc5ccccc5c4)cc3)nc3c4ccc(-c5ccc(-c6ccc7ccccc7c6)cc5)cc4c4cc(-c5ccc(-c6ccc7ccccc7c6)cc5)ccc4c32)cc1 ; c1ccc(-n2c(-c3ccc(-c4nc5ccccc5o4)cc3)nc3c4cc(-c5ccc6ccccc6c5)ccc4c4ccc(-c5ccc6ccccc6c5)cc4c32)cc1 ; c1ccc(-n2c(-c3ccc(-c4nc5ccccc5o4)cc3)nc3c4ccc(-c5ccc6ccccc6c5)cc4c4cc(-c5ccc6ccccc6c5)ccc4c32)cc1 ; c1ccc(-n2c(-c3ccc(-c4cccc5ncoc45)cc3)nc3c4cc(-c5ccc6ccccc6c5)ccc4c4ccc(-c5ccc6ccccc6c5)cc4c32)cc1]

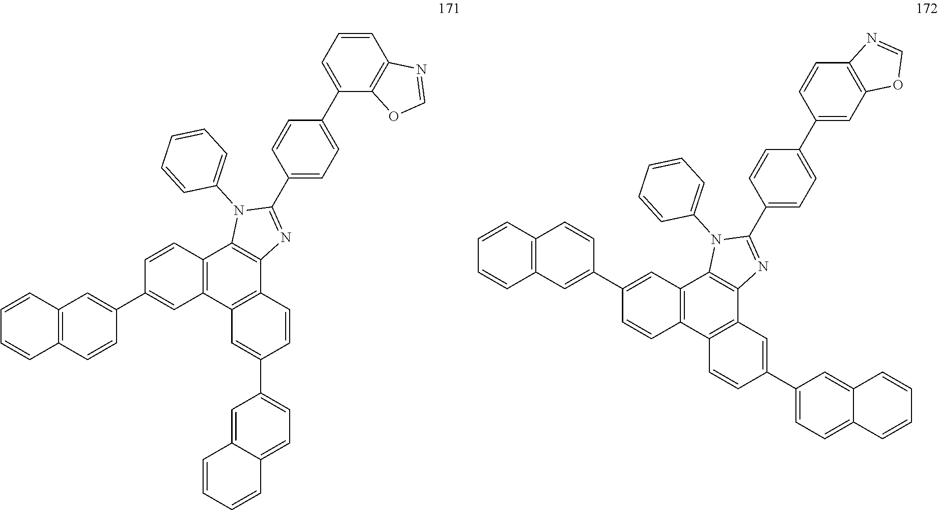 [SMILES: c1ccc(-n2c(-c3ccc(-c4cccc5ncoc45)cc3)nc3c4ccc(-c5ccc6ccccc6c5)cc4c4cc(-c5ccc6ccccc6c5)ccc4c32)cc1 ; c1ccc(-n2c(-c3ccc(-c4ccc5ncoc5c4)cc3)nc3c4cc(-c5ccc6ccccc6c5)ccc4c4ccc(-c5ccc6ccccc6c5)cc4c32)cc1]

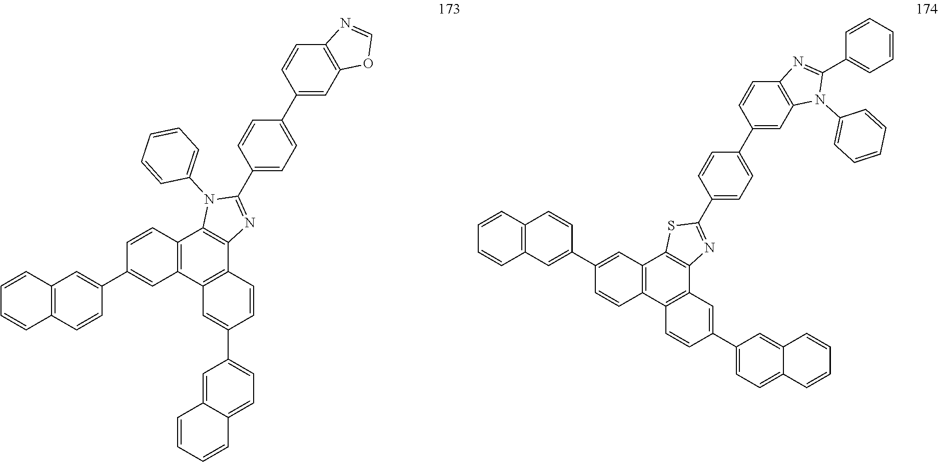 [SMILES: c1ccc(-n2c(-c3ccc(-c4ccc5ncoc5c4)cc3)nc3c4ccc(-c5ccc6ccccc6c5)cc4c4cc(-c5ccc6ccccc6c5)ccc4c32)cc1 ; c1ccc(-c2nc3ccc(-c4ccc(-c5nc6c7cc(-c8ccc9ccccc9c8)ccc7c7ccc(-c8ccc9ccccc9c8)cc7c6s5)cc4)cc3n2-c2ccccc2)cc1]

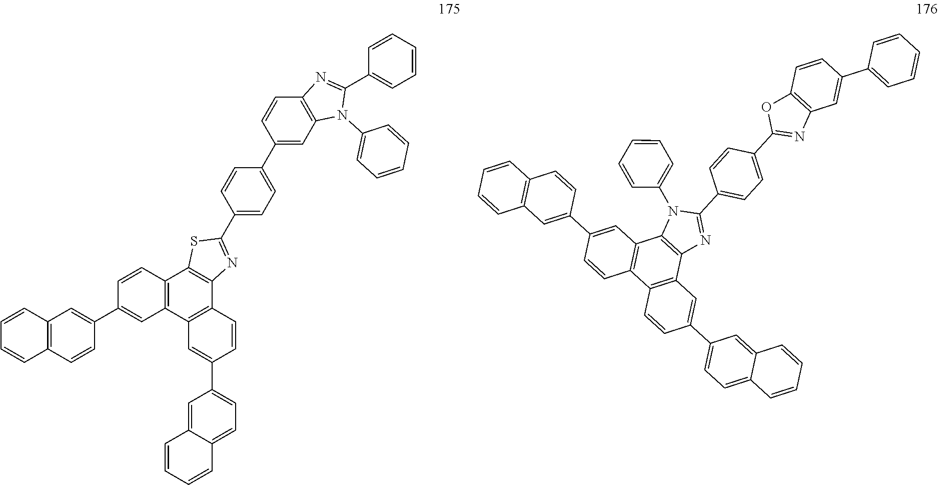 [SMILES: c1ccc(-c2nc3ccc(-c4ccc(-c5nc6c7ccc(-c8ccc9ccccc9c8)cc7c7cc(-c8ccc9ccccc9c8)ccc7c6s5)cc4)cc3n2-c2ccccc2)cc1 ; c1ccc(-c2ccc3oc(-c4ccc(-c5nc6c7cc(-c8ccc9ccccc9c8)ccc7c7ccc(-c8ccc9ccccc9c8)cc7c6n5-c5ccccc5)cc4)nc3c2)cc1]

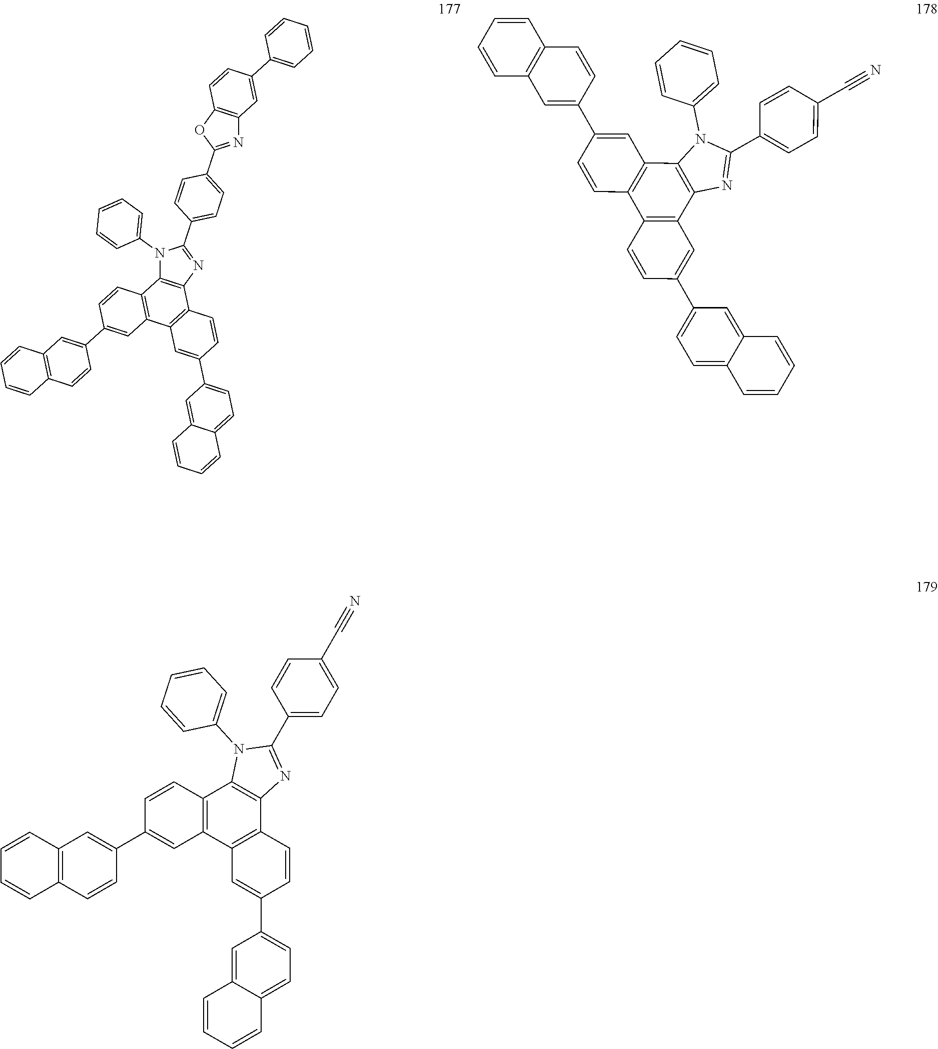 [SMILES: c1ccc(-c2ccc3oc(-c4ccc(-c5nc6c7ccc(-c8ccc9ccccc9c8)cc7c7cc(-c8ccc9ccccc9c8)ccc7c6n5-c5ccccc5)cc4)nc3c2)cc1 ; N#Cc1ccc(-c2nc3c4cc(-c5ccc6ccccc6c5)ccc4c4ccc(-c5ccc6ccccc6c5)cc4c3n2-c2ccccc2)cc1 ; N#Cc1ccc(-c2nc3c4ccc(-c5ccc6ccccc6c5)cc4c4cc(-c5ccc6ccccc6c5)ccc4c3n2-c2ccccc2)cc1]

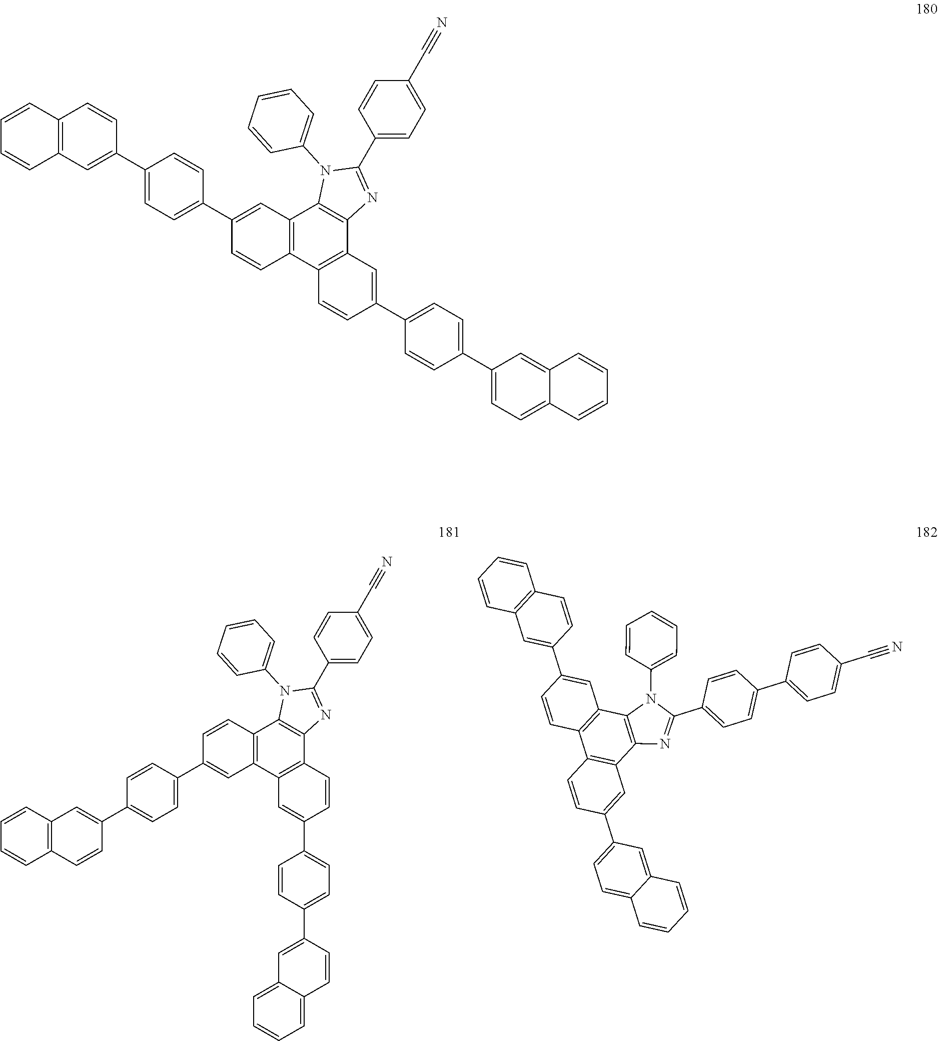 [SMILES: N#Cc1ccc(-c2nc3c4cc(-c5ccc(-c6ccc7ccccc7c6)cc5)ccc4c4ccc(-c5ccc(-c6ccc7ccccc7c6)cc5)cc4c3n2-c2ccccc2)cc1 ; N#Cc1ccc(-c2nc3c4ccc(-c5ccc(-c6ccc7ccccc7c6)cc5)cc4c4cc(-c5ccc(-c6ccc7ccccc7c6)cc5)ccc4c3n2-c2ccccc2)cc1 ; N#Cc1ccc(-c2ccc(-c3nc4c5cc(-c6ccc7ccccc7c6)ccc5c5ccc(-c6ccc7ccccc7c6)cc5c4n3-c3ccccc3)cc2)cc1]

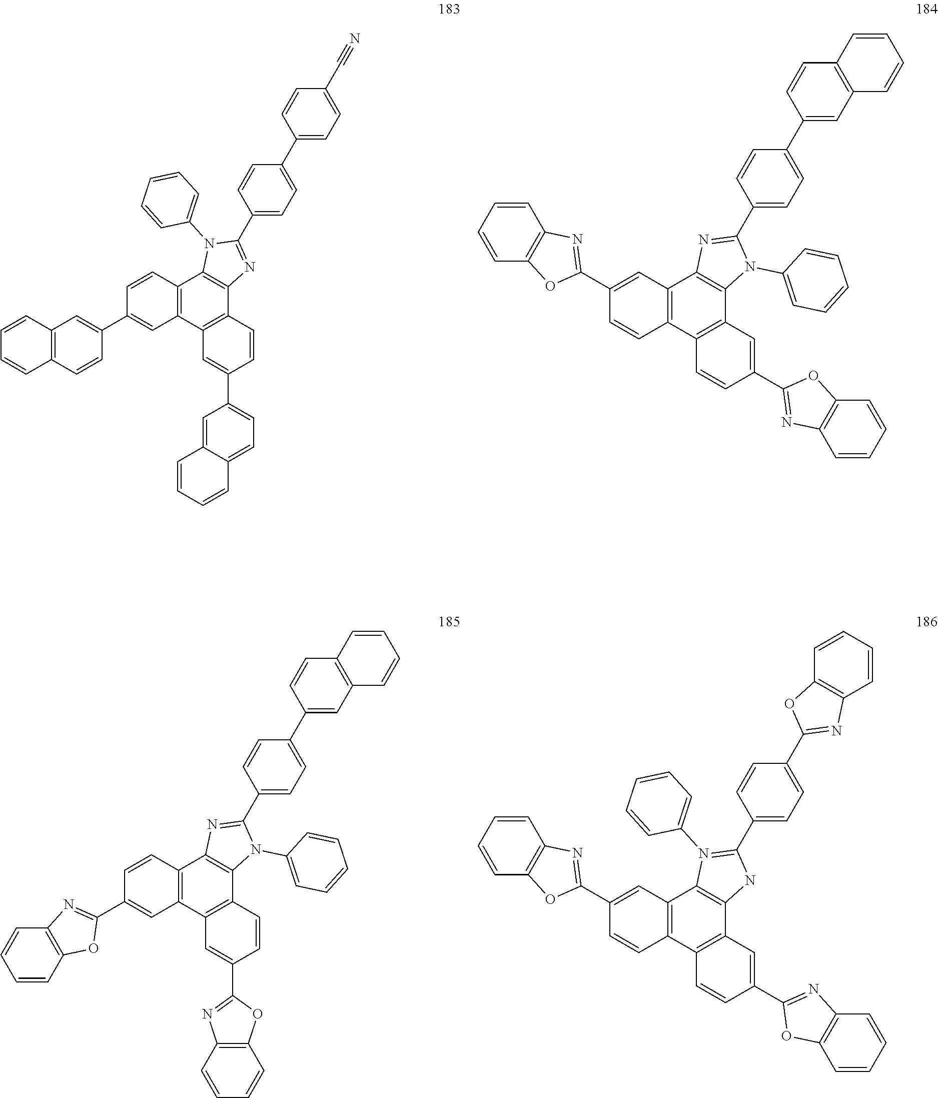

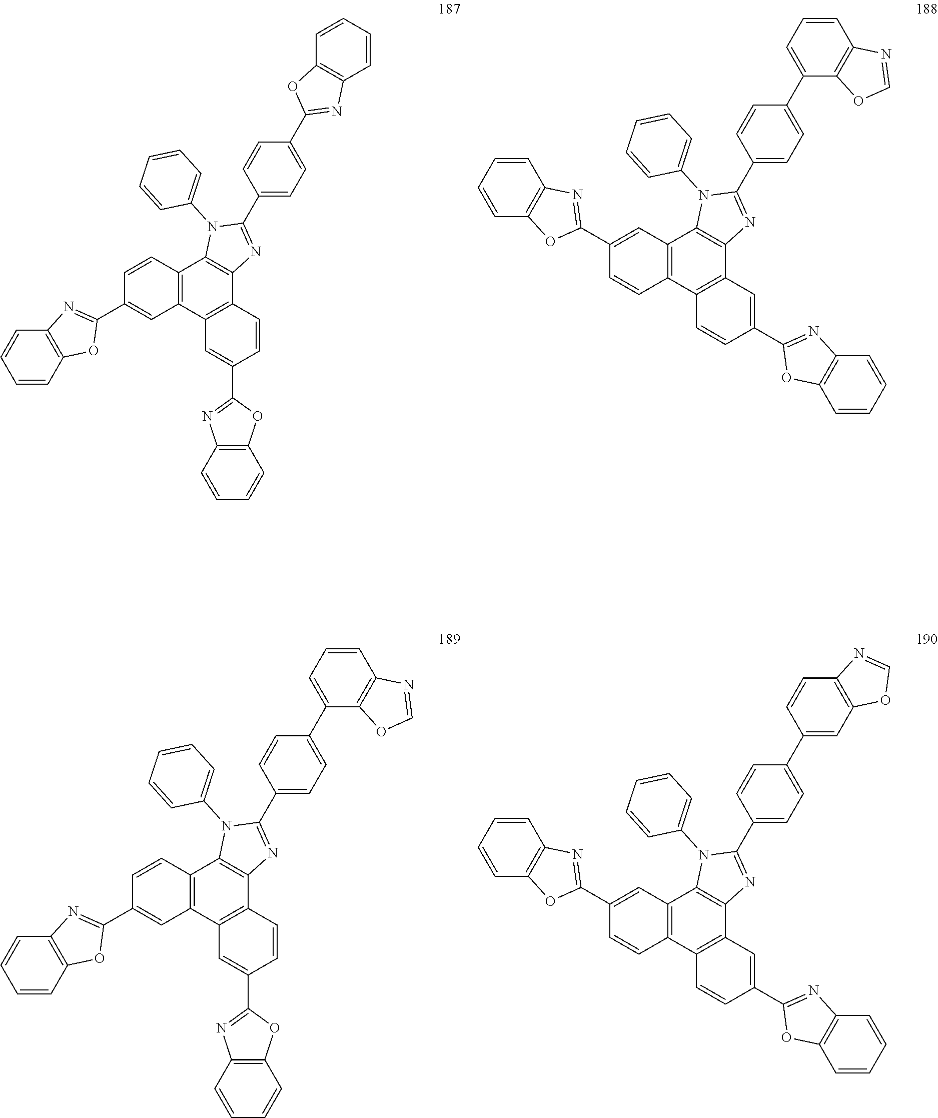 [SMILES: c1ccc(-n2c(-c3ccc(-c4nc5ccccc5o4)cc3)nc3c4ccc(-c5nc6ccccc6o5)cc4c4cc(-c5nc6ccccc6o5)ccc4c32)cc1 ; c1ccc(-n2c(-c3ccc(-c4cccc5ncoc45)cc3)nc3c4cc(-c5nc6ccccc6o5)ccc4c4ccc(-c5nc6ccccc6o5)cc4c32)cc1 ; c1ccc(-n2c(-c3ccc(-c4cccc5ncoc45)cc3)nc3c4ccc(-c5nc6ccccc6o5)cc4c4cc(-c5nc6ccccc6o5)ccc4c32)cc1 ; c1ccc(-n2c(-c3ccc(-c4ccc5ncoc5c4)cc3)nc3c4cc(-c5nc6ccccc6o5)ccc4c4ccc(-c5nc6ccccc6o5)cc4c32)cc1]

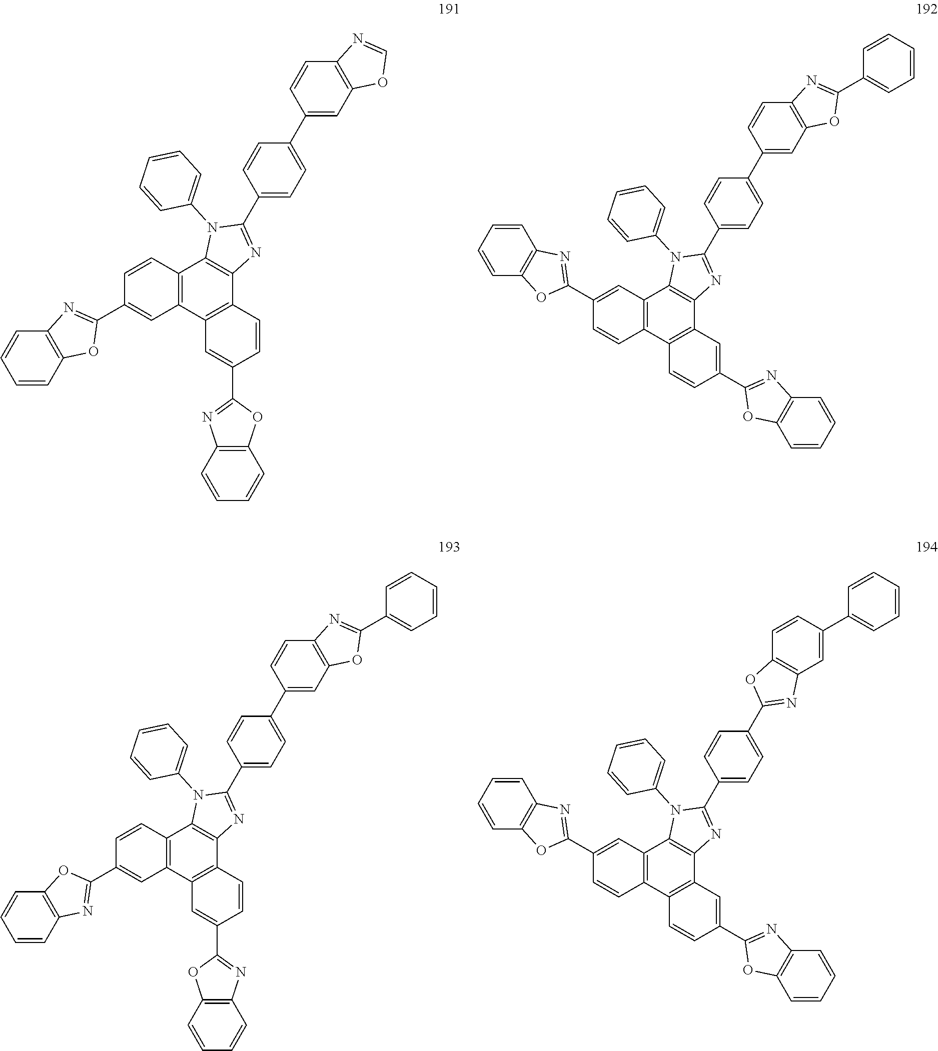 [SMILES: c1ccc(-n2c(-c3ccc(-c4ccc5ncoc5c4)cc3)nc3c4ccc(-c5nc6ccccc6o5)cc4c4cc(-c5nc6ccccc6o5)ccc4c32)cc1 ; c1ccc(-c2nc3ccc(-c4ccc(-c5nc6c7cc(-c8nc9ccccc9o8)ccc7c7ccc(-c8nc9ccccc9o8)cc7c6n5-c5ccccc5)cc4)cc3o2)cc1 ; c1ccc(-c2nc3ccc(-c4ccc(-c5nc6c7ccc(-c8nc9ccccc9o8)cc7c7cc(-c8nc9ccccc9o8)ccc7c6n5-c5ccccc5)cc4)cc3o2)cc1 ; c1ccc(-c2ccc3oc(-c4ccc(-c5nc6c7cc(-c8nc9ccccc9o8)ccc7c7ccc(-c8nc9ccccc9o8)cc7c6n5-c5ccccc5)cc4)nc3c2)cc1]

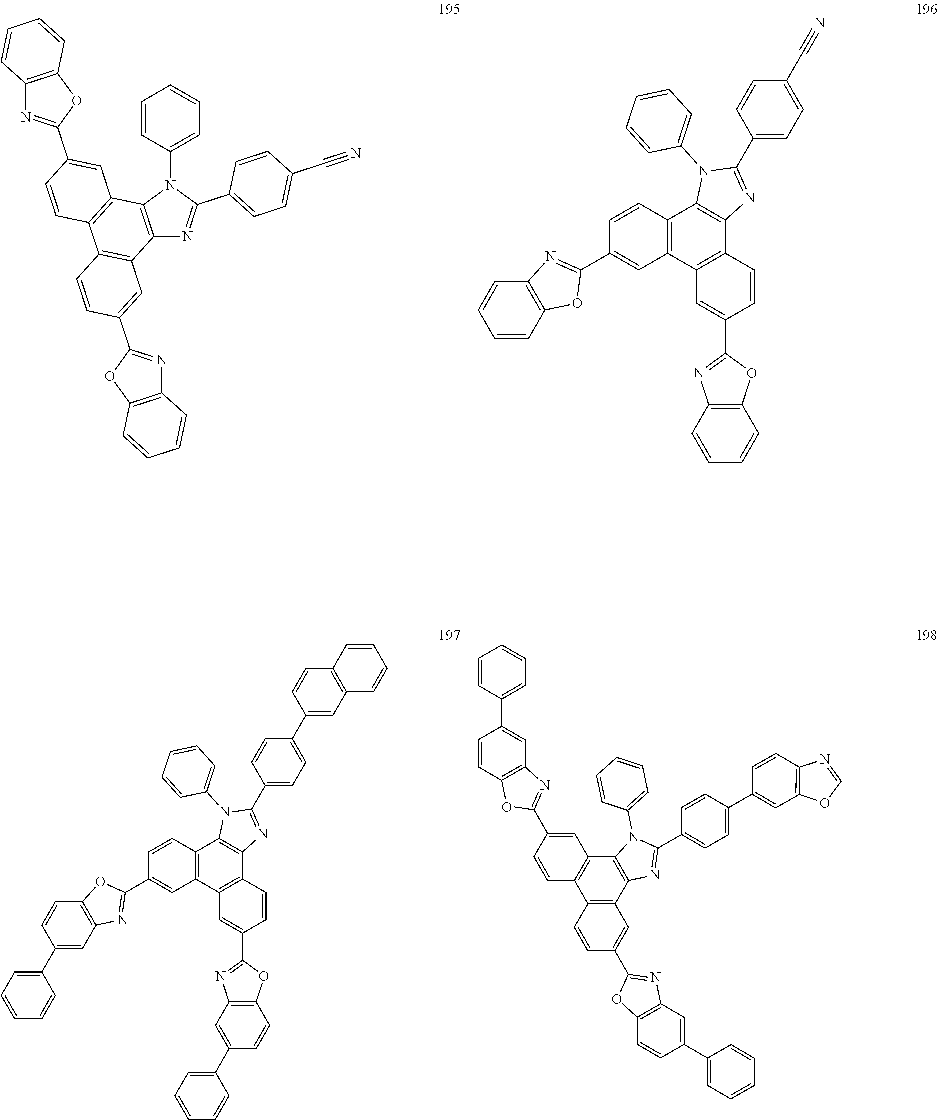 [SMILES: N#Cc1ccc(-c2nc3c4cc(-c5nc6ccccc6o5)ccc4c4ccc(-c5nc6ccccc6o5)cc4c3n2-c2ccccc2)cc1 ; N#Cc1ccc(-c2nc3c4ccc(-c5nc6ccccc6o5)cc4c4cc(-c5nc6ccccc6o5)ccc4c3n2-c2ccccc2)cc1 ; c1ccc(-c2ccc3oc(-c4ccc5c(c4)c4cc(-c6nc7cc(-c8ccccc8)ccc7o6)ccc4c4c5nc(-c5ccc(-c6ccc7ccccc7c6)cc5)n4-c4ccccc4)nc3c2)cc1 ; c1ccc(-c2ccc3oc(-c4ccc5c6ccc(-c7nc8cc(-c9ccccc9)ccc8o7)cc6c6c(nc(-c7ccc(-c8ccc9ncoc9c8)cc7)n6-c6ccccc6)c5c4)nc3c2)cc1]

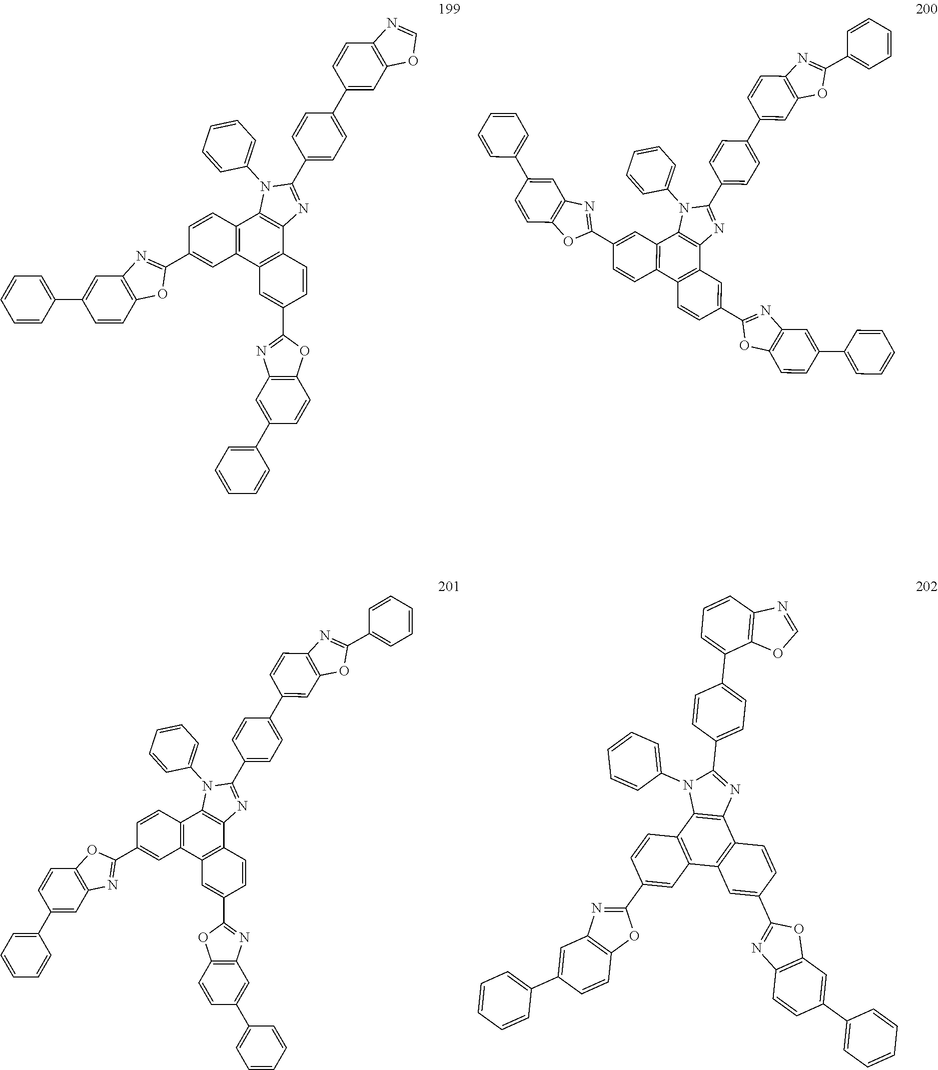

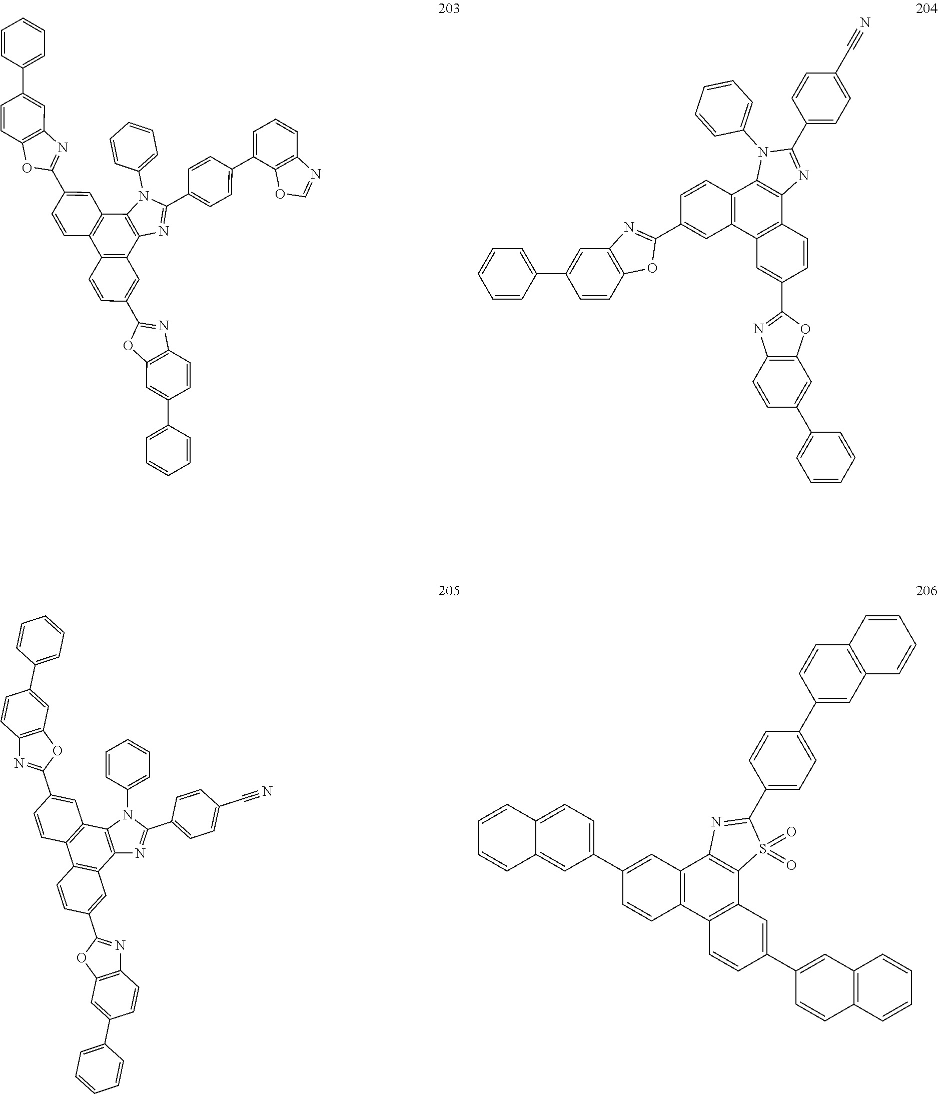 [SMILES: c1ccc(-c2ccc3oc(-c4ccc5c6ccc(-c7nc8ccc(-c9ccccc9)cc8o7)cc6c6nc(-c7ccc(-c8cccc9ncoc89)cc7)n(-c7ccccc7)c6c5c4)nc3c2)cc1 ; N#Cc1ccc(-c2nc3c4ccc(-c5nc6ccc(-c7ccccc7)cc6o5)cc4c4cc(-c5nc6cc(-c7ccccc7)ccc6o5)ccc4c3n2-c2ccccc2)cc1 ; N#Cc1ccc(-c2nc3c4cc(-c5nc6ccc(-c7ccccc7)cc6o5)ccc4c4ccc(-c5nc6ccc(-c7ccccc7)cc6o5)cc4c3n2-c2ccccc2)cc1 ; O=S1(=O)C(c2ccc(-c3ccc4ccccc4c3)cc2)=Nc2c1c1cc(-c3ccc4ccccc4c3)ccc1c1ccc(-c3ccc4ccccc4c3)cc21]

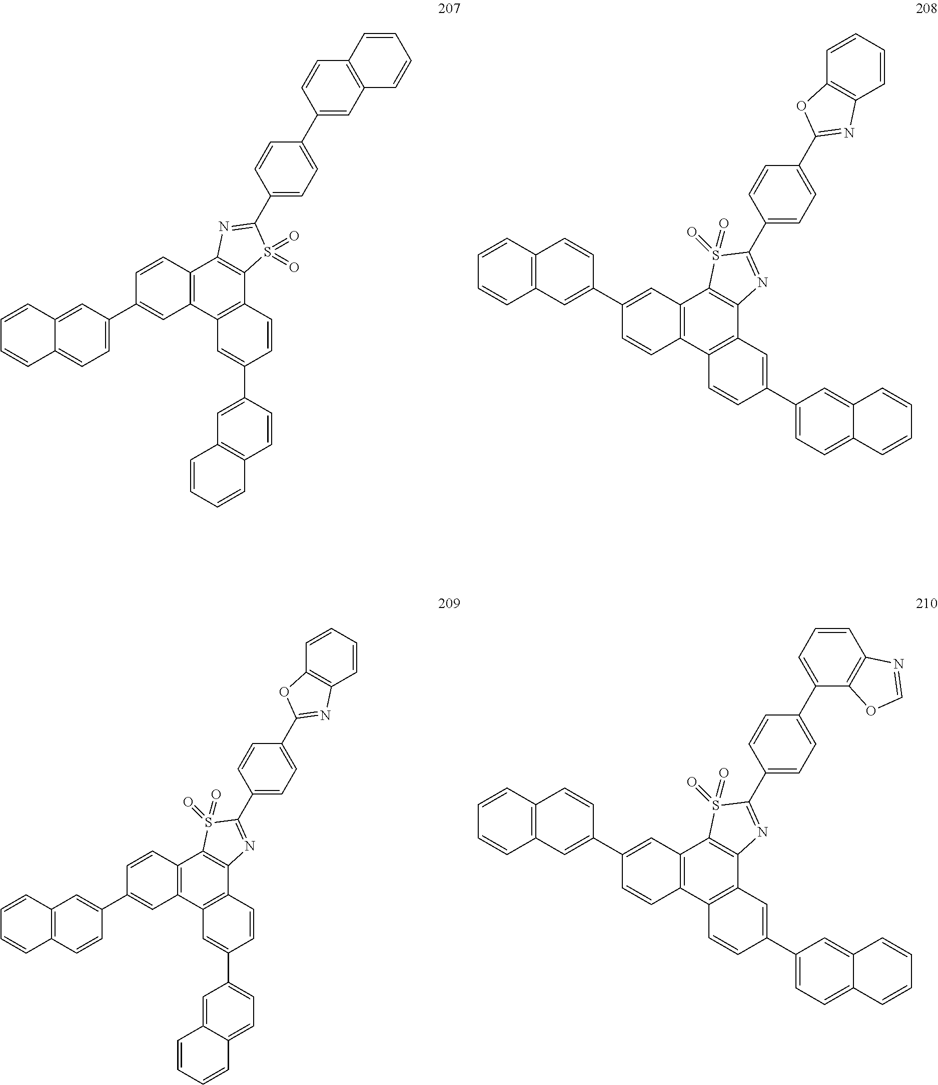 [SMILES: O=S1(=O)C(c2ccc(-c3ccc4ccccc4c3)cc2)=Nc2c1c1ccc(-c3ccc4ccccc4c3)cc1c1cc(-c3ccc4ccccc4c3)ccc21 ; O=S1(=O)C(c2ccc(-c3nc4ccccc4o3)cc2)=Nc2c1c1cc(-c3ccc4ccccc4c3)ccc1c1ccc(-c3ccc4ccccc4c3)cc21 ; O=S1(=O)C(c2ccc(-c3nc4ccccc4o3)cc2)=Nc2c1c1ccc(-c3ccc4ccccc4c3)cc1c1cc(-c3ccc4ccccc4c3)ccc21 ; O=S1(=O)C(c2ccc(-c3cccc4ncoc34)cc2)=Nc2c1c1cc(-c3ccc4ccccc4c3)ccc1c1ccc(-c3ccc4ccccc4c3)cc21]

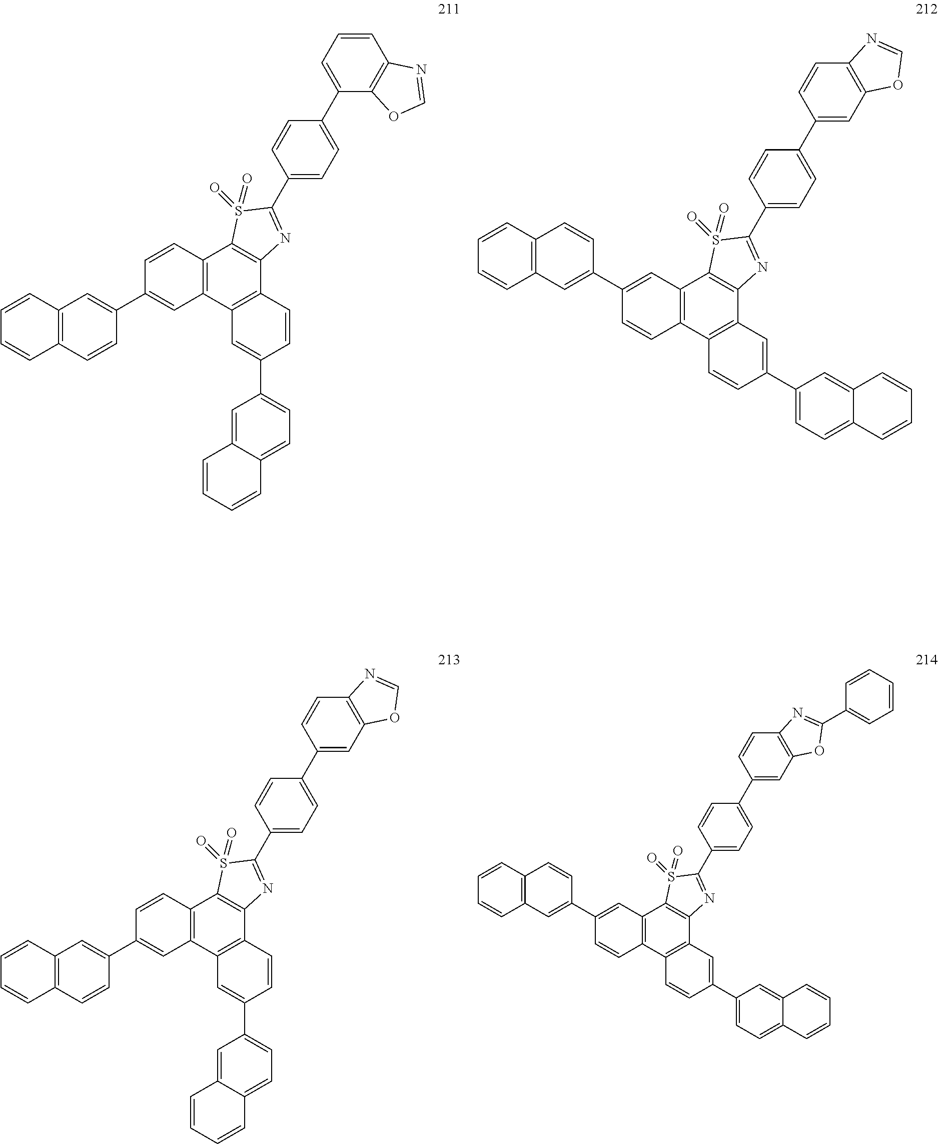 [SMILES: O=S1(=O)C(c2ccc(-c3cccc4ncoc34)cc2)=Nc2c1c1ccc(-c3ccc4ccccc4c3)cc1c1cc(-c3ccc4ccccc4c3)ccc21 ; O=S1(=O)C(c2ccc(-c3ccc4ncoc4c3)cc2)=Nc2c1c1cc(-c3ccc4ccccc4c3)ccc1c1ccc(-c3ccc4ccccc4c3)cc21 ; O=S1(=O)C(c2ccc(-c3ccc4ncoc4c3)cc2)=Nc2c1c1ccc(-c3ccc4ccccc4c3)cc1c1cc(-c3ccc4ccccc4c3)ccc21 ; O=S1(=O)C(c2ccc(-c3ccc4nc(-c5ccccc5)oc4c3)cc2)=Nc2c1c1cc(-c3ccc4ccccc4c3)ccc1c1ccc(-c3ccc4ccccc4c3)cc21]

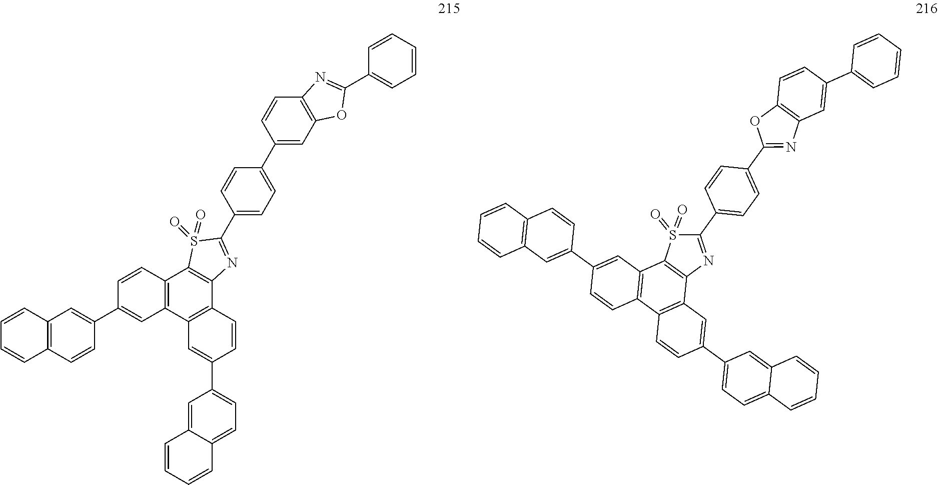 [SMILES: O=S1(=O)C(c2ccc(-c3ccc4nc(-c5ccccc5)oc4c3)cc2)=Nc2c1c1ccc(-c3ccc4ccccc4c3)cc1c1cc(-c3ccc4ccccc4c3)ccc21 ; O=S1(=O)C(c2ccc(-c3nc4cc(-c5ccccc5)ccc4o3)cc2)=Nc2c1c1cc(-c3ccc4ccccc4c3)ccc1c1ccc(-c3ccc4ccccc4c3)cc21]

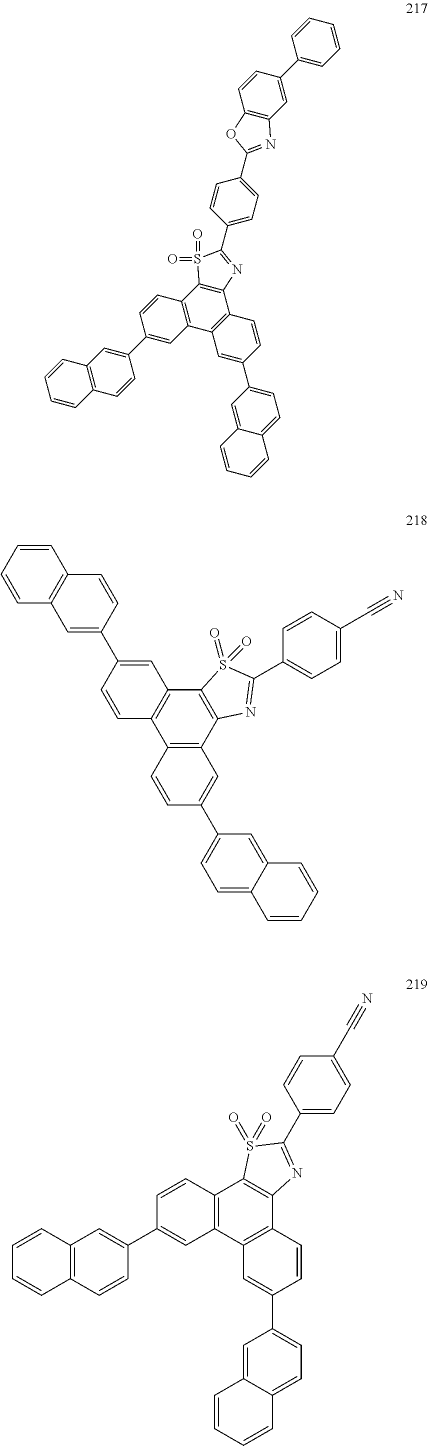 [SMILES: O=S1(=O)C(c2ccc(-c3nc4cc(-c5ccccc5)ccc4o3)cc2)=Nc2c1c1ccc(-c3ccc4ccccc4c3)cc1c1cc(-c3ccc4ccccc4c3)ccc21 ; N#Cc1ccc(C2=Nc3c(c4cc(-c5ccc6ccccc6c5)ccc4c4ccc(-c5ccc6ccccc6c5)cc34)S2(=O)=O)cc1 ; N#Cc1ccc(C2=Nc3c(c4ccc(-c5ccc6ccccc6c5)cc4c4cc(-c5ccc6ccccc6c5)ccc34)S2(=O)=O)cc1]

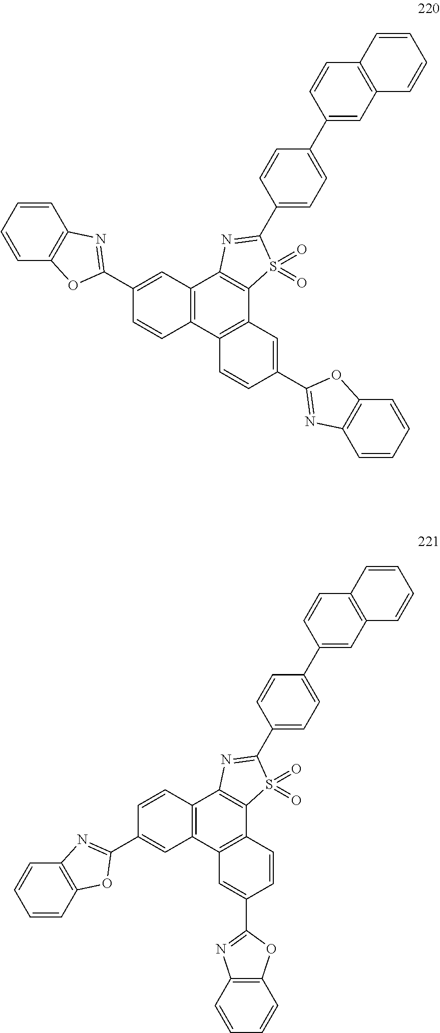 [SMILES: O=S1(=O)C(c2ccc(-c3ccc4ccccc4c3)cc2)=Nc2c1c1cc(-c3nc4ccccc4o3)ccc1c1ccc(-c3nc4ccccc4o3)cc21 ; O=S1(=O)C(c2ccc(-c3ccc4ccccc4c3)cc2)=Nc2c1c1ccc(-c3nc4ccccc4o3)cc1c1cc(-c3nc4ccccc4o3)ccc21]

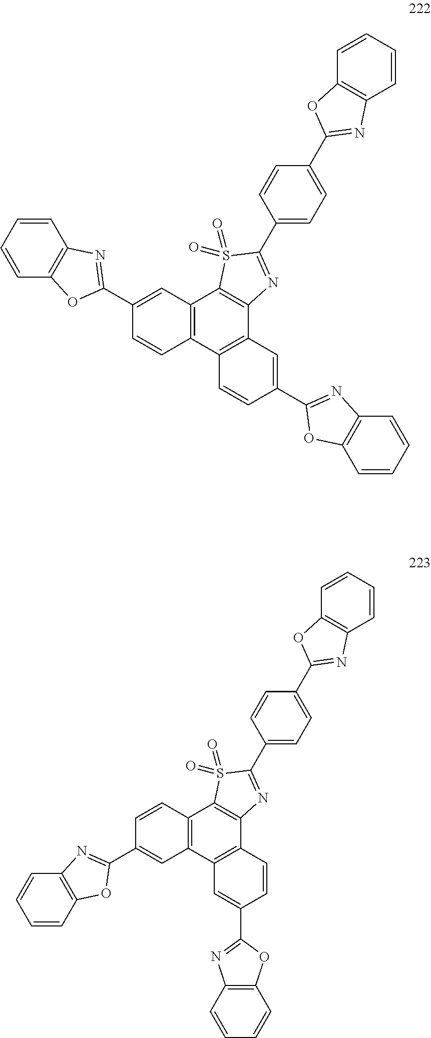 [SMILES: O=S1(=O)C(c2ccc(-c3nc4ccccc4o3)cc2)=Nc2c1c1cc(-c3nc4ccccc4o3)ccc1c1ccc(-c3nc4ccccc4o3)cc21 ; O=S1(=O)C(c2ccc(-c3nc4ccccc4o3)cc2)=Nc2c1c1ccc(-c3nc4ccccc4o3)cc1c1cc(-c3nc4ccccc4o3)ccc21]

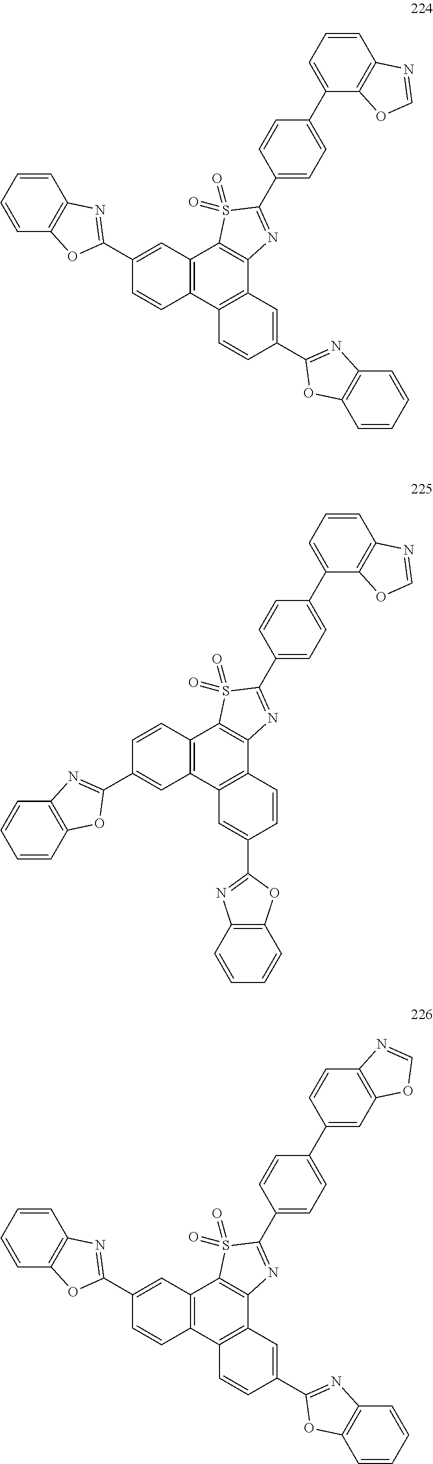 [SMILES: O=S1(=O)C(c2ccc(-c3cccc4ncoc34)cc2)=Nc2c1c1cc(-c3nc4ccccc4o3)ccc1c1ccc(-c3nc4ccccc4o3)cc21 ; O=S1(=O)C(c2ccc(-c3cccc4ncoc34)cc2)=Nc2c1c1ccc(-c3nc4ccccc4o3)cc1c1cc(-c3nc4ccccc4o3)ccc21 ; O=S1(=O)C(c2ccc(-c3ccc4ncoc4c3)cc2)=Nc2c1c1cc(-c3nc4ccccc4o3)ccc1c1ccc(-c3nc4ccccc4o3)cc21]

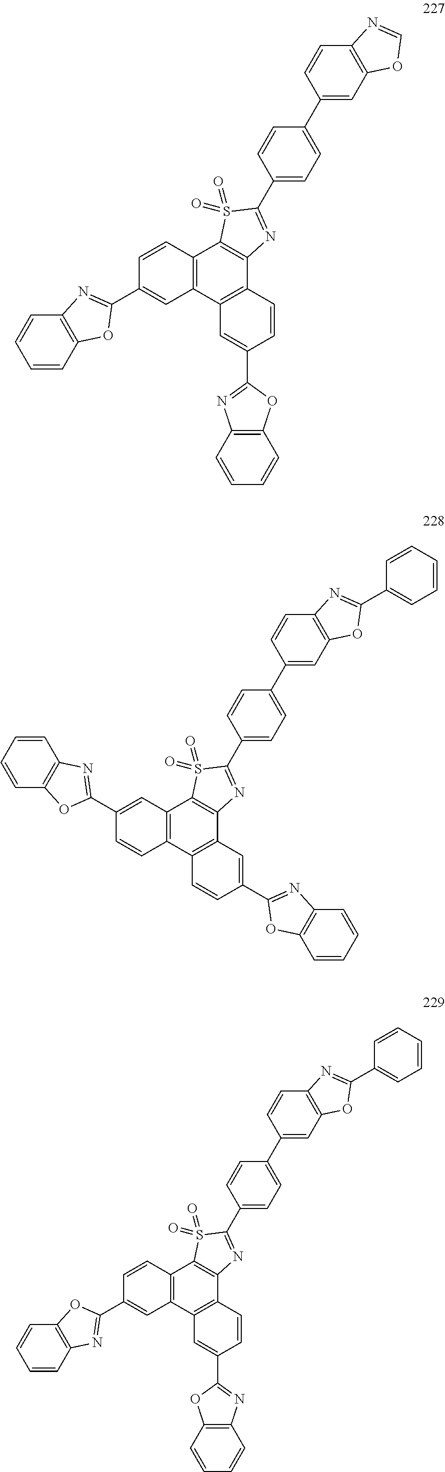 [SMILES: O=S1(=O)C(c2ccc(-c3ccc4ncoc4c3)cc2)=Nc2c1c1ccc(-c3nc4ccccc4o3)cc1c1cc(-c3nc4ccccc4o3)ccc21 ; O=S1(=O)C(c2ccc(-c3ccc4nc(-c5ccccc5)oc4c3)cc2)=Nc2c1c1cc(-c3nc4ccccc4o3)ccc1c1ccc(-c3nc4ccccc4o3)cc21 ; O=S1(=O)C(c2ccc(-c3ccc4nc(-c5ccccc5)oc4c3)cc2)=Nc2c1c1ccc(-c3nc4ccccc4o3)cc1c1cc(-c3nc4ccccc4o3)ccc21]

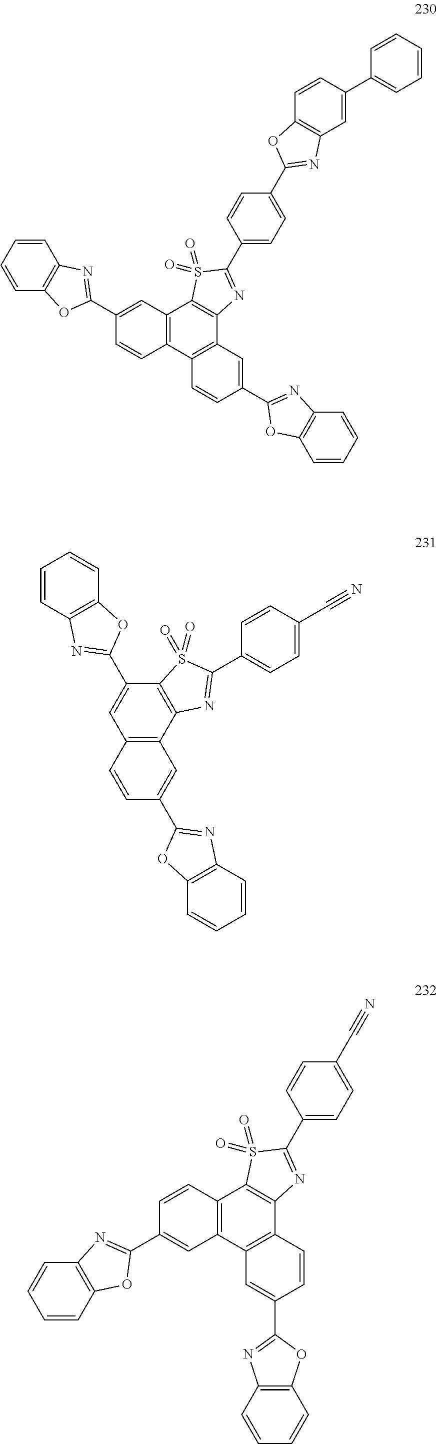 [SMILES: O=S1(=O)C(c2ccc(-c3nc4cc(-c5ccccc5)ccc4o3)cc2)=Nc2c1c1cc(-c3nc4ccccc4o3)ccc1c1ccc(-c3nc4ccccc4o3)cc21 ; N#Cc1ccc(C2=Nc3c(c(-c4nc5ccccc5o4)cc4ccc(-c5nc6ccccc6o5)cc34)S2(=O)=O)cc1 ; N#Cc1ccc(C2=Nc3c(c4ccc(-c5nc6ccccc6o5)cc4c4cc(-c5nc6ccccc6o5)ccc34)S2(=O)=O)cc1]

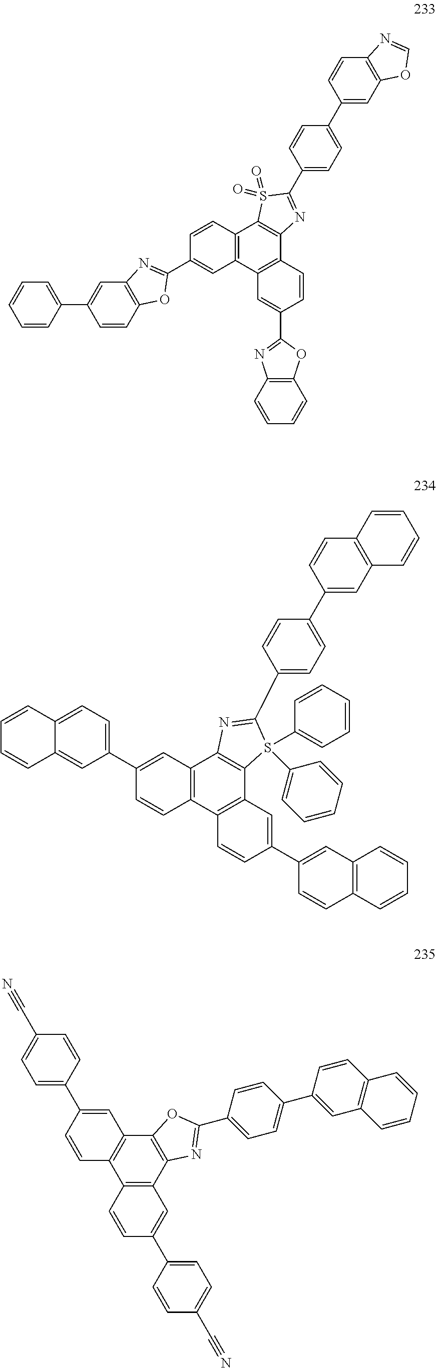 [SMILES: O=S1(=O)C(c2ccc(-c3ccc4ncoc4c3)cc2)=Nc2c1c1ccc(-c3nc4cc(-c5ccccc5)ccc4o3)cc1c1cc(-c3nc4ccccc4o3)ccc21 ; c1ccc(S2(c3ccccc3)C(c3ccc(-c4ccc5ccccc5c4)cc3)=Nc3c2c2cc(-c4ccc5ccccc5c4)ccc2c2ccc(-c4ccc5ccccc5c4)cc32)cc1 ; N#Cc1ccc(-c2ccc3c4ccc(-c5ccc(C#N)cc5)cc4c4oc(-c5ccc(-c6ccc7ccccc7c6)cc5)nc4c3c2)cc1]

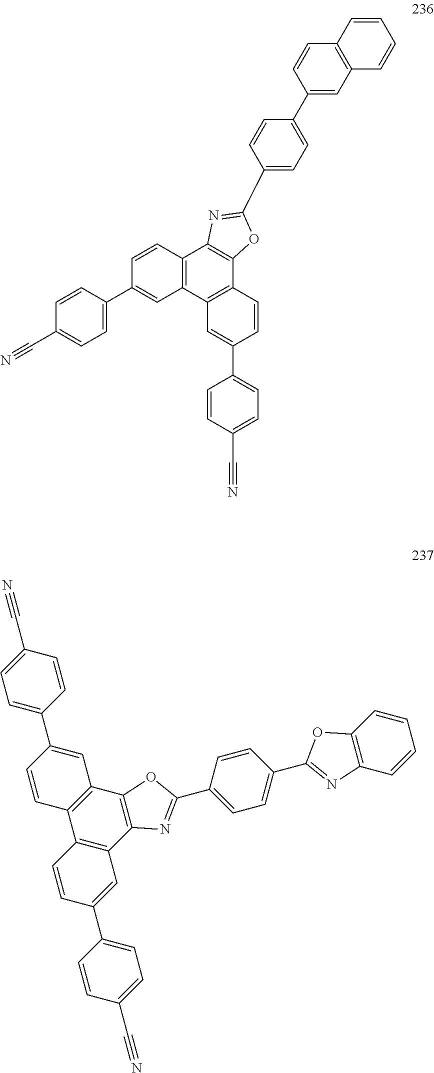 [SMILES: N#Cc1ccc(-c2ccc3c(c2)c2cc(-c4ccc(C#N)cc4)ccc2c2oc(-c4ccc(-c5ccc6ccccc6c5)cc4)nc32)cc1 ; N#Cc1ccc(-c2ccc3c4ccc(-c5ccc(C#N)cc5)cc4c4oc(-c5ccc(-c6nc7ccccc7o6)cc5)nc4c3c2)cc1]

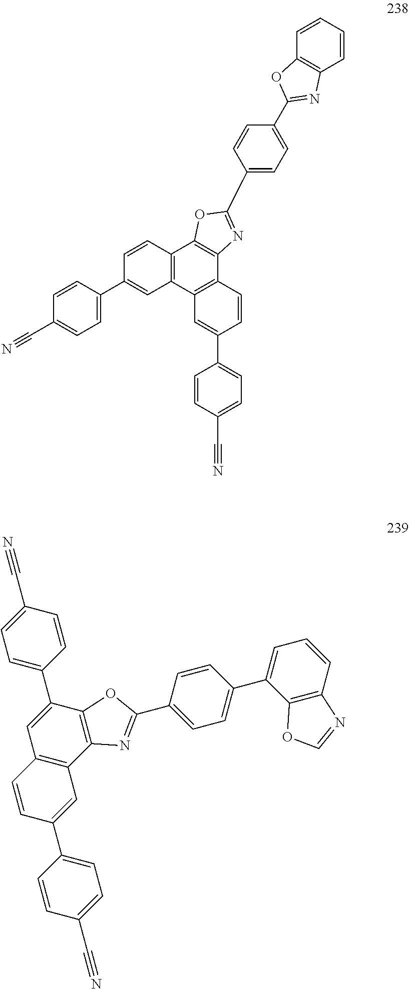 [SMILES: N#Cc1ccc(-c2ccc3c(c2)c2cc(-c4ccc(C#N)cc4)ccc2c2oc(-c4ccc(-c5nc6ccccc6o5)cc4)nc32)cc1 ; N#Cc1ccc(-c2ccc3cc(-c4ccc(C#N)cc4)c4oc(-c5ccc(-c6cccc7ncoc67)cc5)nc4c3c2)cc1]

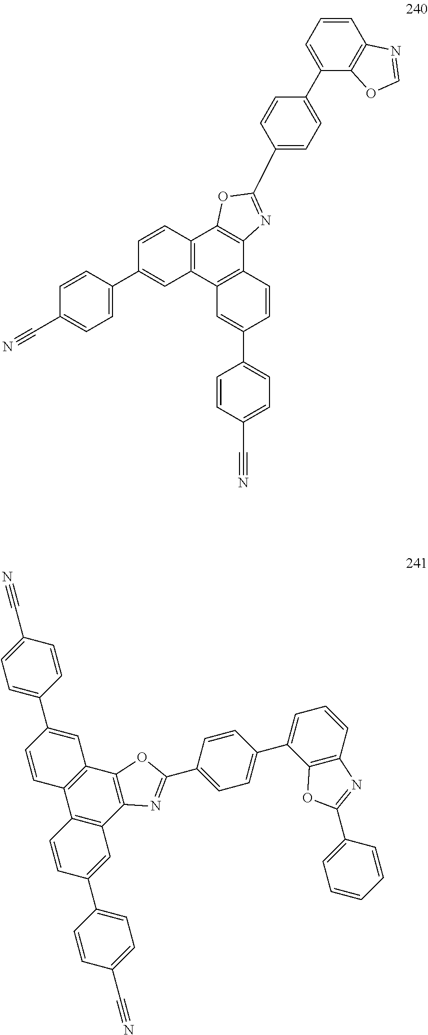 [SMILES: N#Cc1ccc(-c2ccc3c(c2)c2cc(-c4ccc(C#N)cc4)ccc2c2oc(-c4ccc(-c5cccc6ncoc56)cc4)nc32)cc1 ; N#Cc1ccc(-c2ccc3c4ccc(-c5ccc(C#N)cc5)cc4c4oc(-c5ccc(-c6cccc7nc(-c8ccccc8)oc67)cc5)nc4c3c2)cc1]

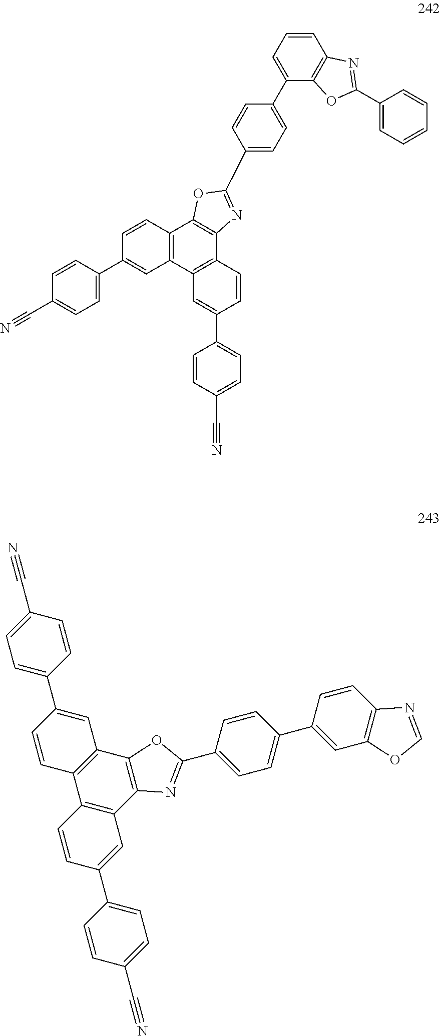 [SMILES: N#Cc1ccc(-c2ccc3c(c2)c2cc(-c4ccc(C#N)cc4)ccc2c2oc(-c4ccc(-c5cccc6nc(-c7ccccc7)oc56)cc4)nc32)cc1 ; N#Cc1ccc(-c2ccc3c4ccc(-c5ccc(C#N)cc5)cc4c4oc(-c5ccc(-c6ccc7ncoc7c6)cc5)nc4c3c2)cc1]

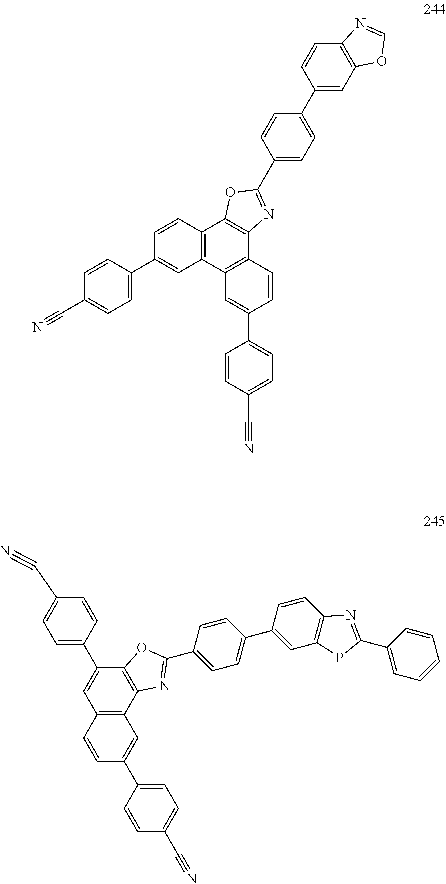

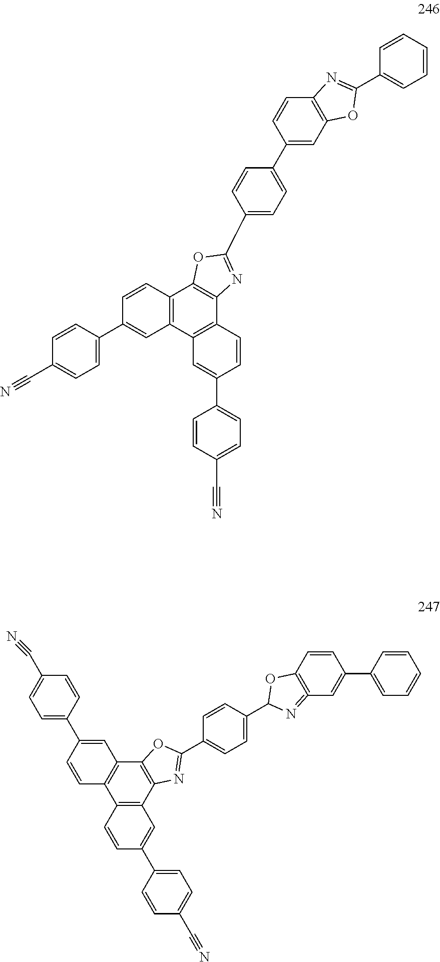

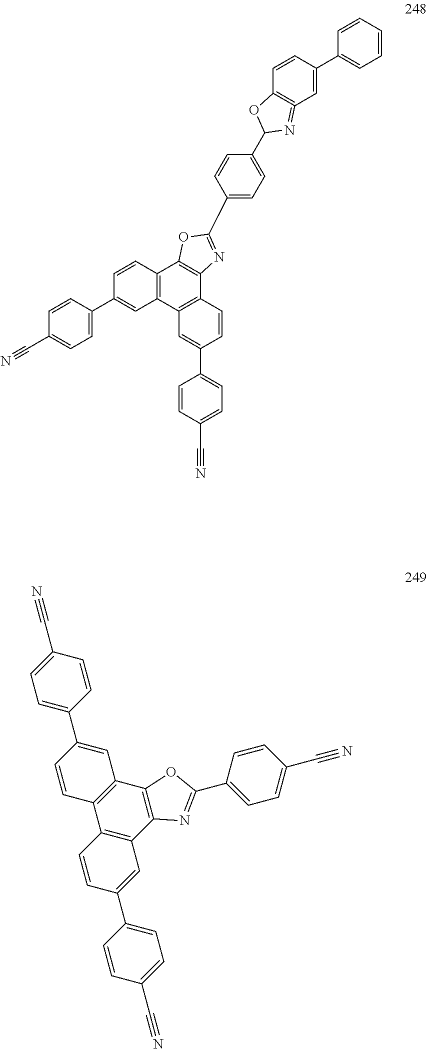

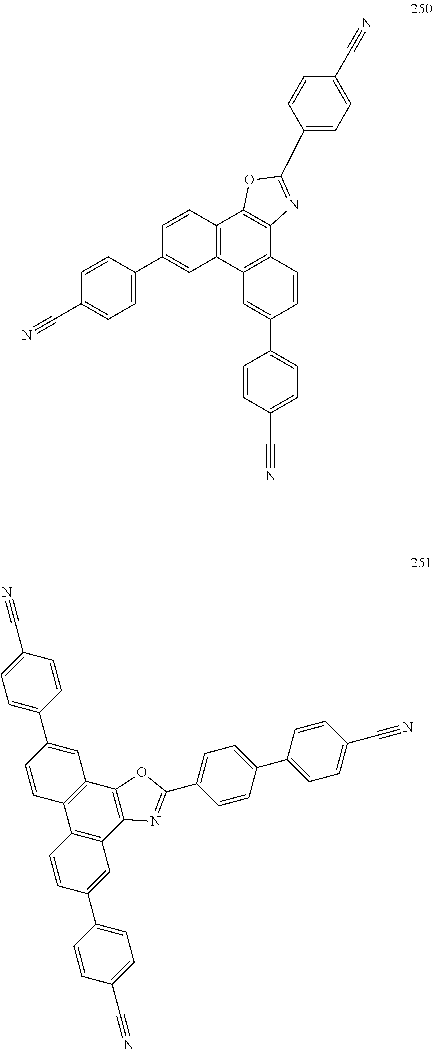 [SMILES: N#Cc1ccc(-c2ccc3c(c2)c2cc(-c4ccc(C#N)cc4)ccc2c2oc(-c4ccc(C#N)cc4)nc32)cc1 ; N#Cc1ccc(-c2ccc(-c3nc4c5cc(-c6ccc(C#N)cc6)ccc5c5ccc(-c6ccc(C#N)cc6)cc5c4o3)cc2)cc1]

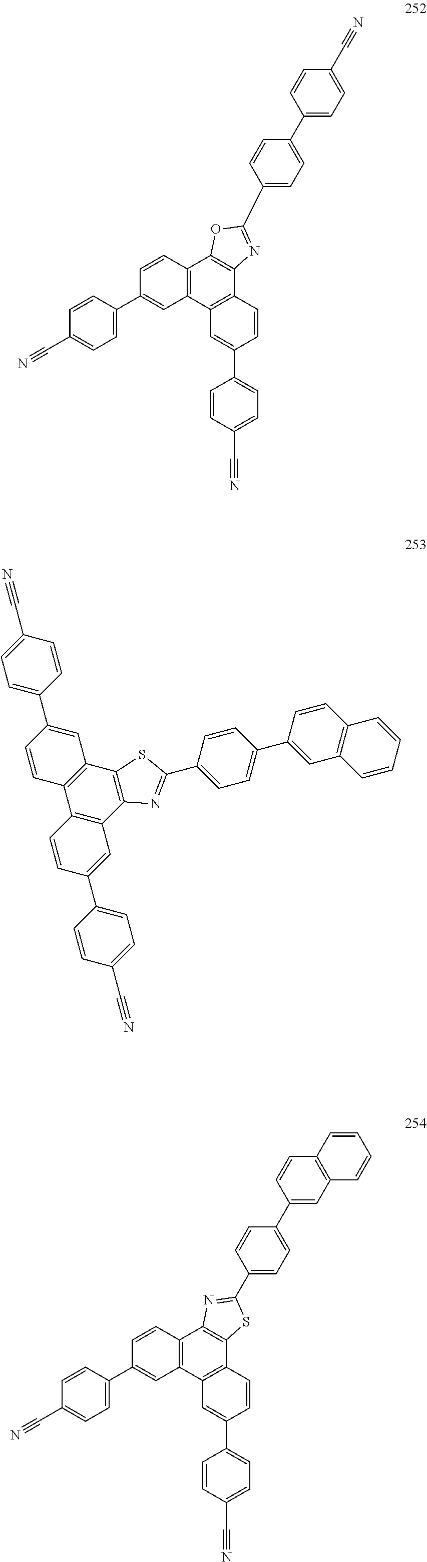 [SMILES: N#Cc1ccc(-c2ccc(-c3nc4c5ccc(-c6ccc(C#N)cc6)cc5c5cc(-c6ccc(C#N)cc6)ccc5c4o3)cc2)cc1 ; N#Cc1ccc(-c2ccc3c4ccc(-c5ccc(C#N)cc5)cc4c4sc(-c5ccc(-c6ccc7ccccc7c6)cc5)nc4c3c2)cc1 ; N#Cc1ccc(-c2ccc3c(c2)c2cc(-c4ccc(C#N)cc4)ccc2c2sc(-c4ccc(-c5ccc6ccccc6c5)cc4)nc32)cc1]

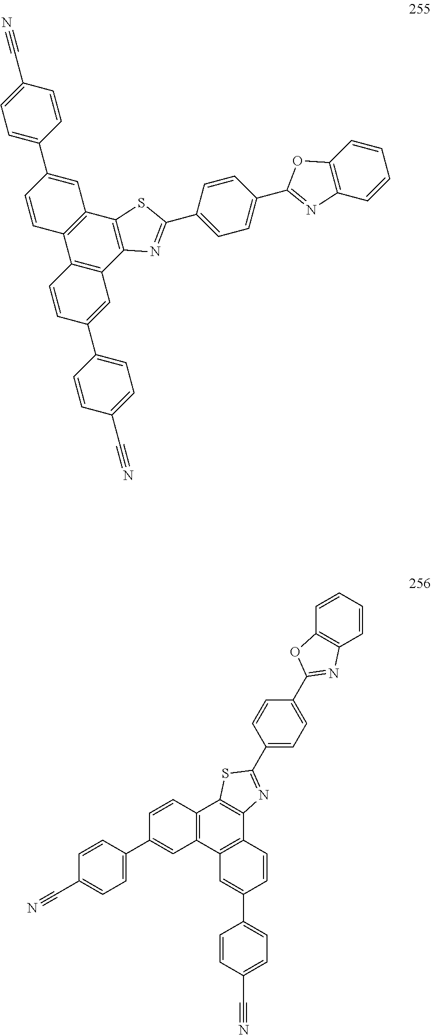 [SMILES: N#Cc1ccc(-c2ccc3c4ccc(-c5ccc(C#N)cc5)cc4c4sc(-c5ccc(-c6nc7ccccc7o6)cc5)nc4c3c2)cc1 ; N#Cc1ccc(-c2ccc3c(c2)c2cc(-c4ccc(C#N)cc4)ccc2c2sc(-c4ccc(-c5nc6ccccc6o5)cc4)nc32)cc1]

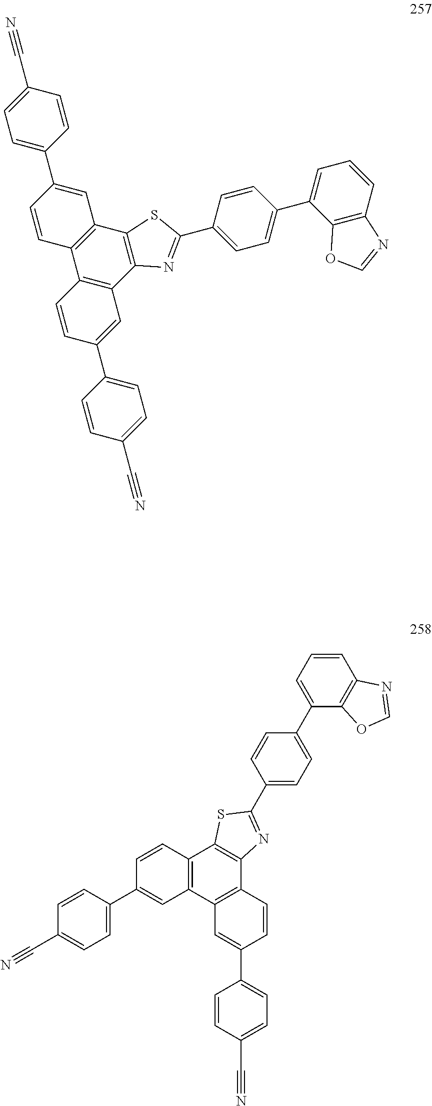 [SMILES: N#Cc1ccc(-c2ccc3c4ccc(-c5ccc(C#N)cc5)cc4c4sc(-c5ccc(-c6cccc7ncoc67)cc5)nc4c3c2)cc1 ; N#Cc1ccc(-c2ccc3c(c2)c2cc(-c4ccc(C#N)cc4)ccc2c2sc(-c4ccc(-c5cccc6ncoc56)cc4)nc32)cc1]

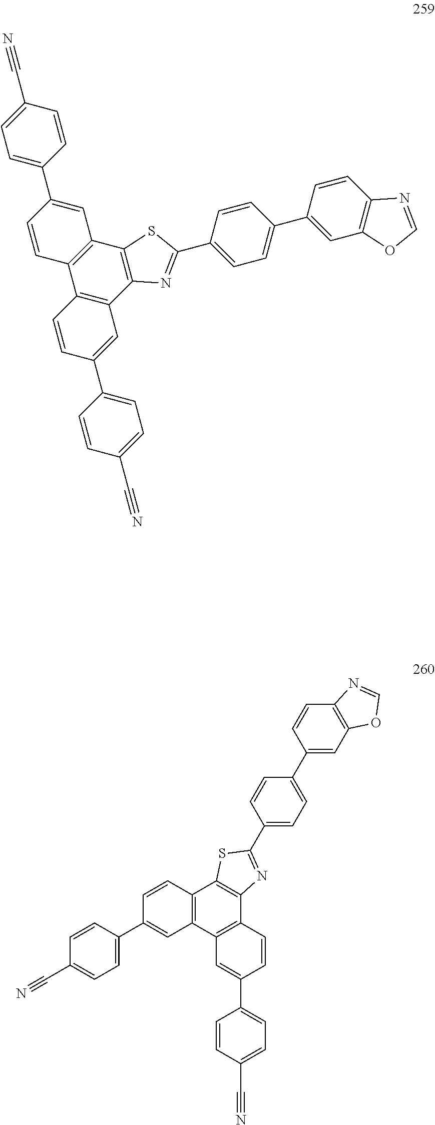 [SMILES: N#Cc1ccc(-c2ccc3c4ccc(-c5ccc(C#N)cc5)cc4c4sc(-c5ccc(-c6ccc7ncoc7c6)cc5)nc4c3c2)cc1 ; N#Cc1ccc(-c2ccc3c(c2)c2cc(-c4ccc(C#N)cc4)ccc2c2sc(-c4ccc(-c5ccc6ncoc6c5)cc4)nc32)cc1]

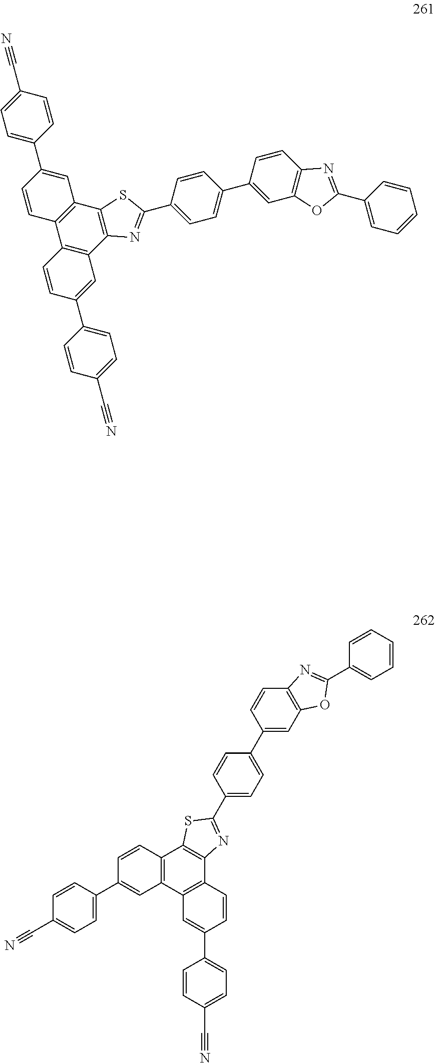 [SMILES: N#Cc1ccc(-c2ccc3c4ccc(-c5ccc(C#N)cc5)cc4c4sc(-c5ccc(-c6ccc7nc(-c8ccccc8)oc7c6)cc5)nc4c3c2)cc1 ; N#Cc1ccc(-c2ccc3c(c2)c2cc(-c4ccc(C#N)cc4)ccc2c2sc(-c4ccc(-c5ccc6nc(-c7ccccc7)oc6c5)cc4)nc32)cc1]

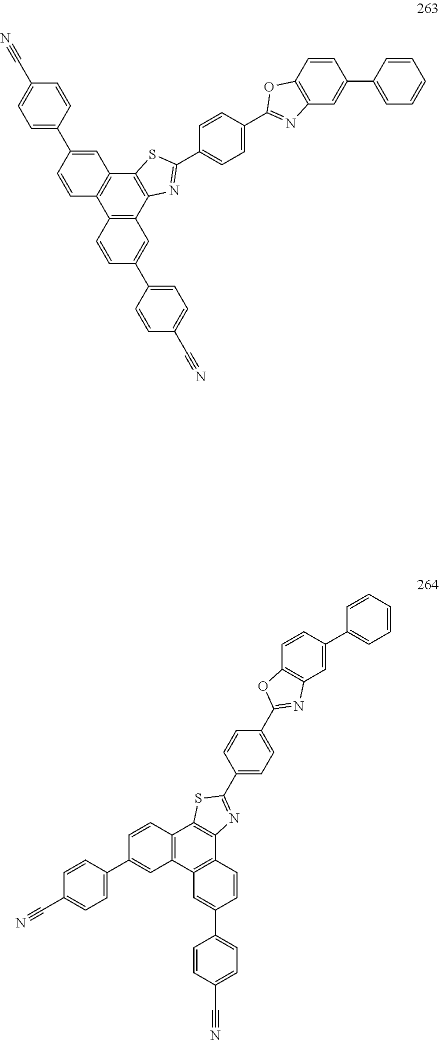 [SMILES: N#Cc1ccc(-c2ccc3c4ccc(-c5ccc(C#N)cc5)cc4c4sc(-c5ccc(-c6nc7cc(-c8ccccc8)ccc7o6)cc5)nc4c3c2)cc1 ; N#Cc1ccc(-c2ccc3c(c2)c2cc(-c4ccc(C#N)cc4)ccc2c2sc(-c4ccc(-c5nc6cc(-c7ccccc7)ccc6o5)cc4)nc32)cc1]

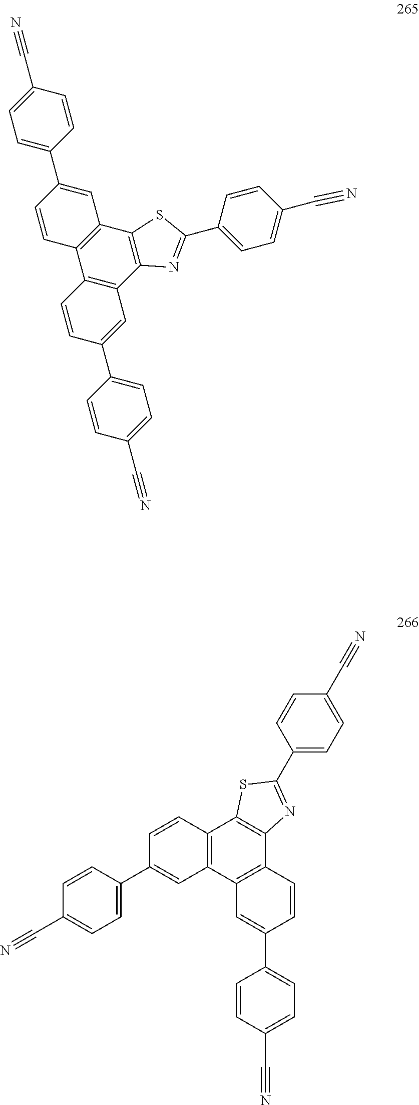 [SMILES: N#Cc1ccc(-c2ccc3c4ccc(-c5ccc(C#N)cc5)cc4c4sc(-c5ccc(C#N)cc5)nc4c3c2)cc1 ; N#Cc1ccc(-c2ccc3c(c2)c2cc(-c4ccc(C#N)cc4)ccc2c2sc(-c4ccc(C#N)cc4)nc32)cc1]

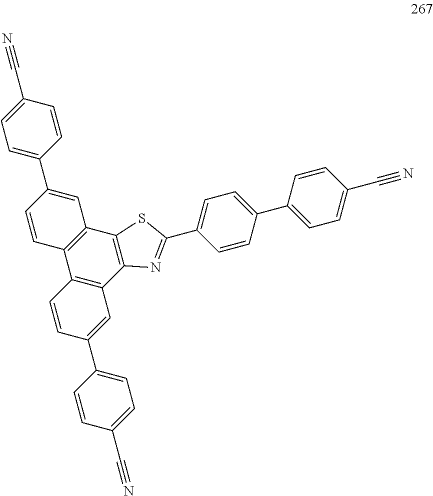 [SMILES: N#Cc1ccc(-c2ccc(-c3nc4c5cc(-c6ccc(C#N)cc6)ccc5c5ccc(-c6ccc(C#N)cc6)cc5c4s3)cc2)cc1]

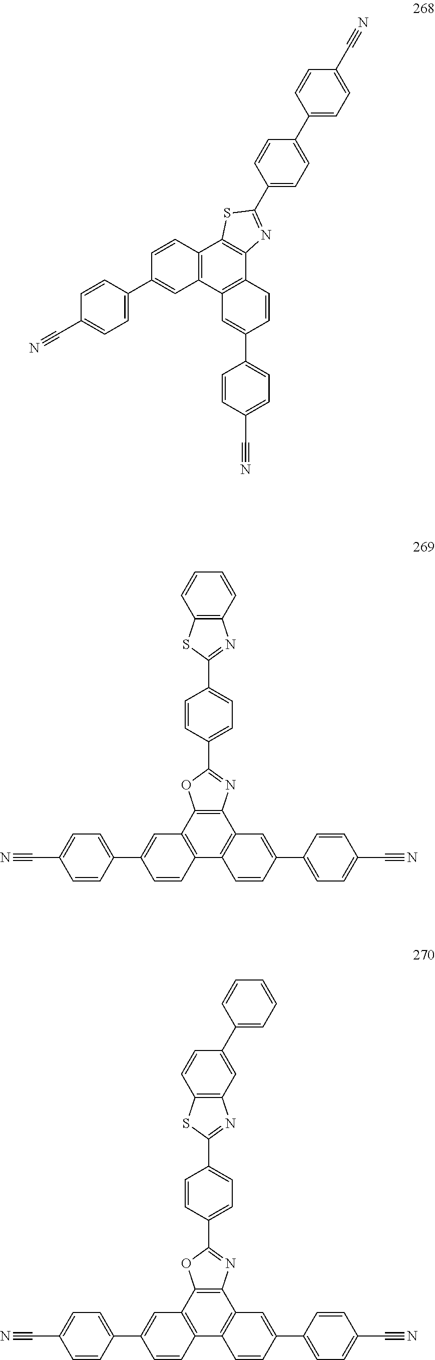 [SMILES: N#Cc1ccc(-c2ccc(-c3nc4c5ccc(-c6ccc(C#N)cc6)cc5c5cc(-c6ccc(C#N)cc6)ccc5c4s3)cc2)cc1 ; N#Cc1ccc(-c2ccc3c4ccc(-c5ccc(C#N)cc5)cc4c4oc(-c5ccc(-c6nc7ccccc7s6)cc5)nc4c3c2)cc1 ; N#Cc1ccc(-c2ccc3c4ccc(-c5ccc(C#N)cc5)cc4c4oc(-c5ccc(-c6nc7cc(-c8ccccc8)ccc7s6)cc5)nc4c3c2)cc1]

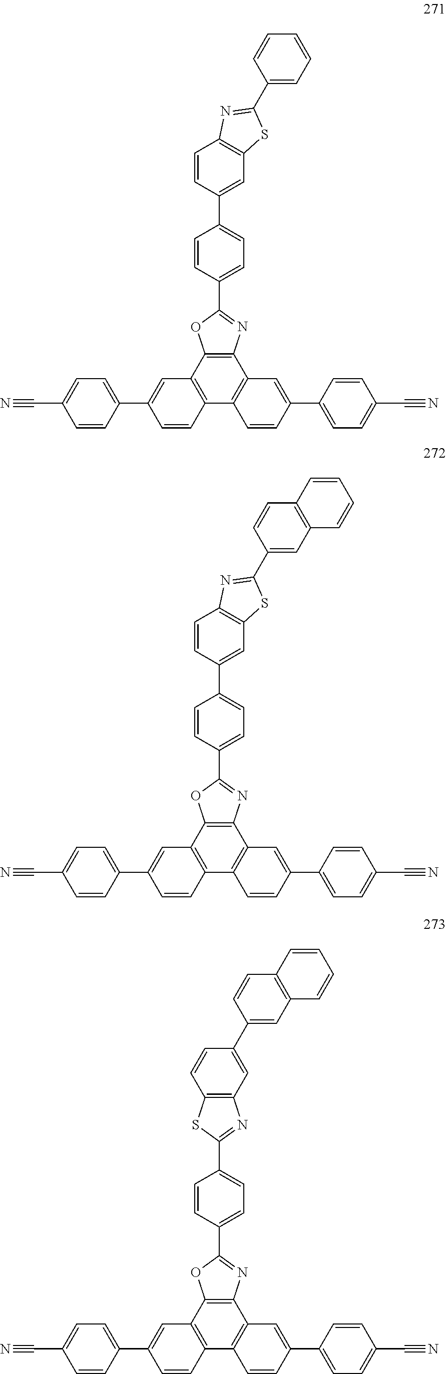 [SMILES: N#Cc1ccc(-c2ccc3c4ccc(-c5ccc(C#N)cc5)cc4c4oc(-c5ccc(-c6ccc7nc(-c8ccccc8)sc7c6)cc5)nc4c3c2)cc1 ; N#Cc1ccc(-c2ccc3c4ccc(-c5ccc(C#N)cc5)cc4c4oc(-c5ccc(-c6ccc7nc(-c8ccc9ccccc9c8)sc7c6)cc5)nc4c3c2)cc1 ; N#Cc1ccc(-c2ccc3c4ccc(-c5ccc(C#N)cc5)cc4c4oc(-c5ccc(-c6nc7cc(-c8ccc9ccccc9c8)ccc7s6)cc5)nc4c3c2)cc1]

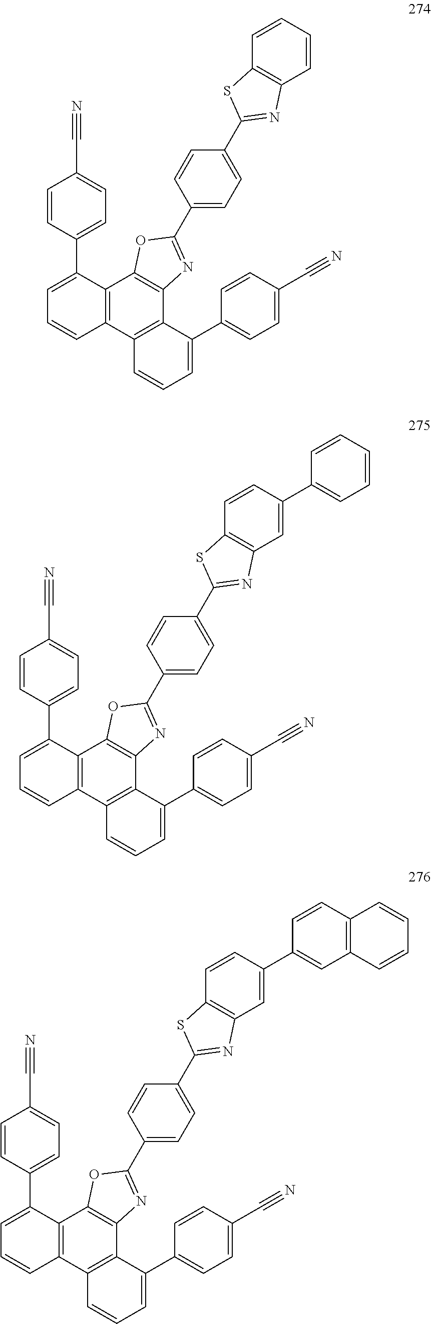 [SMILES: N#Cc1ccc(-c2cccc3c4cccc(-c5ccc(C#N)cc5)c4c4oc(-c5ccc(-c6nc7ccccc7s6)cc5)nc4c23)cc1 ; N#Cc1ccc(-c2cccc3c4cccc(-c5ccc(C#N)cc5)c4c4oc(-c5ccc(-c6nc7cc(-c8ccccc8)ccc7s6)cc5)nc4c23)cc1 ; N#Cc1ccc(-c2cccc3c4cccc(-c5ccc(C#N)cc5)c4c4oc(-c5ccc(-c6nc7cc(-c8ccc9ccccc9c8)ccc7s6)cc5)nc4c23)cc1]

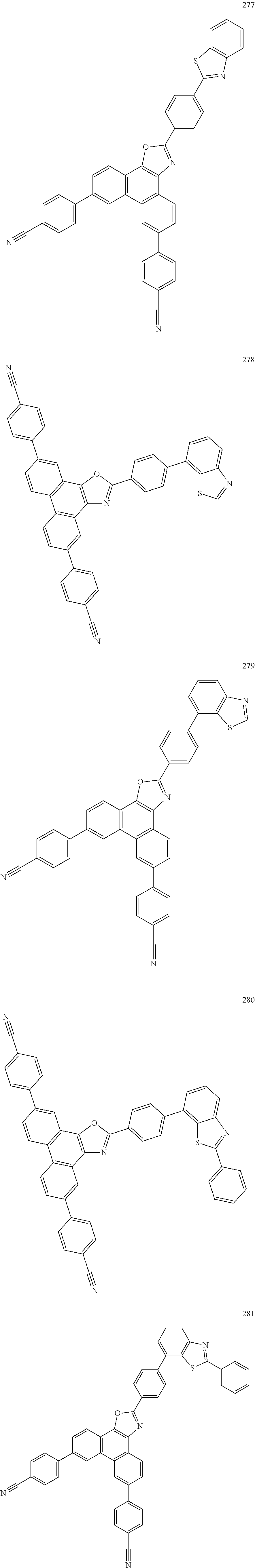 [SMILES: N#Cc1ccc(-c2ccc3c(c2)c2cc(-c4ccc(C#N)cc4)ccc2c2oc(-c4ccc(-c5nc6ccccc6s5)cc4)nc32)cc1 ; N#Cc1ccc(-c2ccc3c4ccc(-c5ccc(C#N)cc5)cc4c4oc(-c5ccc(-c6cccc7ncsc67)cc5)nc4c3c2)cc1 ; N#Cc1ccc(-c2ccc3c(c2)c2cc(-c4ccc(C#N)cc4)ccc2c2oc(-c4ccc(-c5cccc6ncsc56)cc4)nc32)cc1 ; N#Cc1ccc(-c2ccc3c4ccc(-c5ccc(C#N)cc5)cc4c4oc(-c5ccc(-c6cccc7nc(-c8ccccc8)sc67)cc5)nc4c3c2)cc1 ; N#Cc1ccc(-c2ccc3c(c2)c2cc(-c4ccc(C#N)cc4)ccc2c2oc(-c4ccc(-c5cccc6nc(-c7ccccc7)sc56)cc4)nc32)cc1]

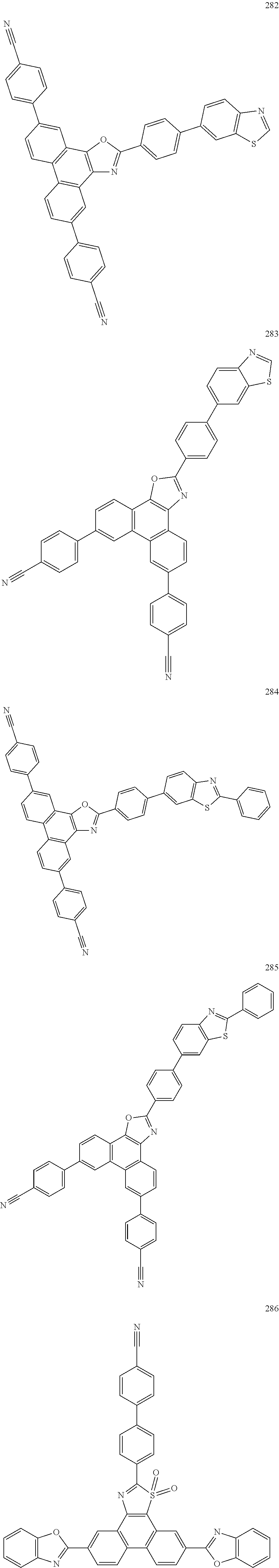 [SMILES: N#Cc1ccc(-c2ccc3c4ccc(-c5ccc(C#N)cc5)cc4c4oc(-c5ccc(-c6ccc7ncsc7c6)cc5)nc4c3c2)cc1 ; N#Cc1ccc(-c2ccc3c(c2)c2cc(-c4ccc(C#N)cc4)ccc2c2oc(-c4ccc(-c5ccc6ncsc6c5)cc4)nc32)cc1 ; N#Cc1ccc(-c2ccc3c4ccc(-c5ccc(C#N)cc5)cc4c4oc(-c5ccc(-c6ccc7nc(-c8ccccc8)sc7c6)cc5)nc4c3c2)cc1 ; N#Cc1ccc(-c2ccc3c(c2)c2cc(-c4ccc(C#N)cc4)ccc2c2oc(-c4ccc(-c5ccc6nc(-c7ccccc7)sc6c5)cc4)nc32)cc1 ; N#Cc1ccc(-c2ccc(C3=Nc4c(c5cc(-c6nc7ccccc7o6)ccc5c5ccc(-c6nc7ccccc7o6)cc45)S3(=O)=O)cc2)cc1]

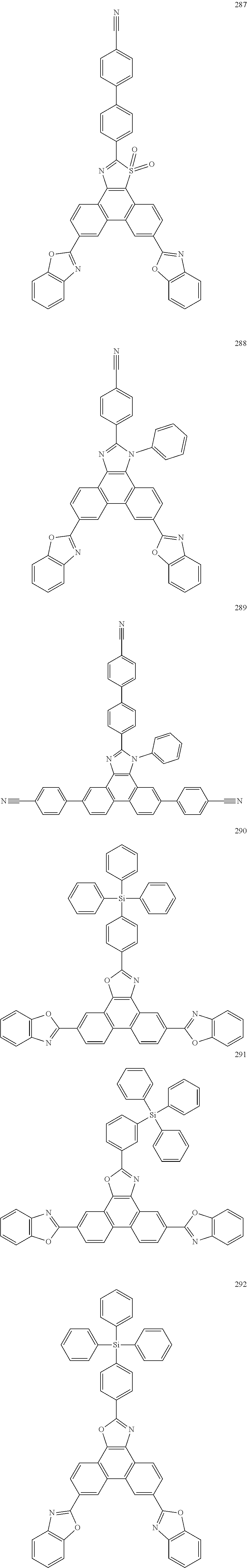

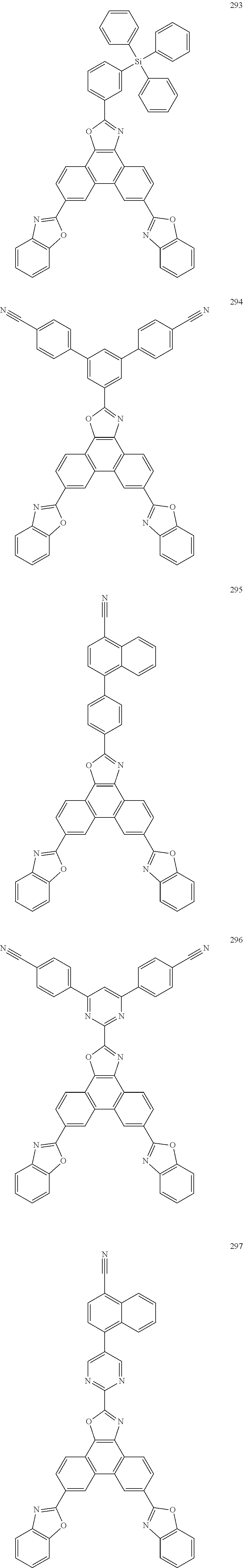 [SMILES: c1ccc([Si](c2ccccc2)(c2ccccc2)c2cccc(-c3nc4c5ccc(-c6nc7ccccc7o6)cc5c5cc(-c6nc7ccccc7o6)ccc5c4o3)c2)cc1 ; N#Cc1ccc(-c2cc(-c3ccc(C#N)cc3)cc(-c3nc4c5ccc(-c6nc7ccccc7o6)cc5c5cc(-c6nc7ccccc7o6)ccc5c4o3)c2)cc1 ; N#Cc1ccc(-c2ccc(-c3nc4c5ccc(-c6nc7ccccc7o6)cc5c5cc(-c6nc7ccccc7o6)ccc5c4o3)cc2)c2ccccc12 ; N#Cc1ccc(-c2cc(-c3ccc(C#N)cc3)nc(-c3nc4c5ccc(-c6nc7ccccc7o6)cc5c5cc(-c6nc7ccccc7o6)ccc5c4o3)n2)cc1 ; N#Cc1ccc(-c2cnc(-c3nc4c5ccc(-c6nc7ccccc7o6)cc5c5cc(-c6nc7ccccc7o6)ccc5c4o3)nc2)c2ccccc12]

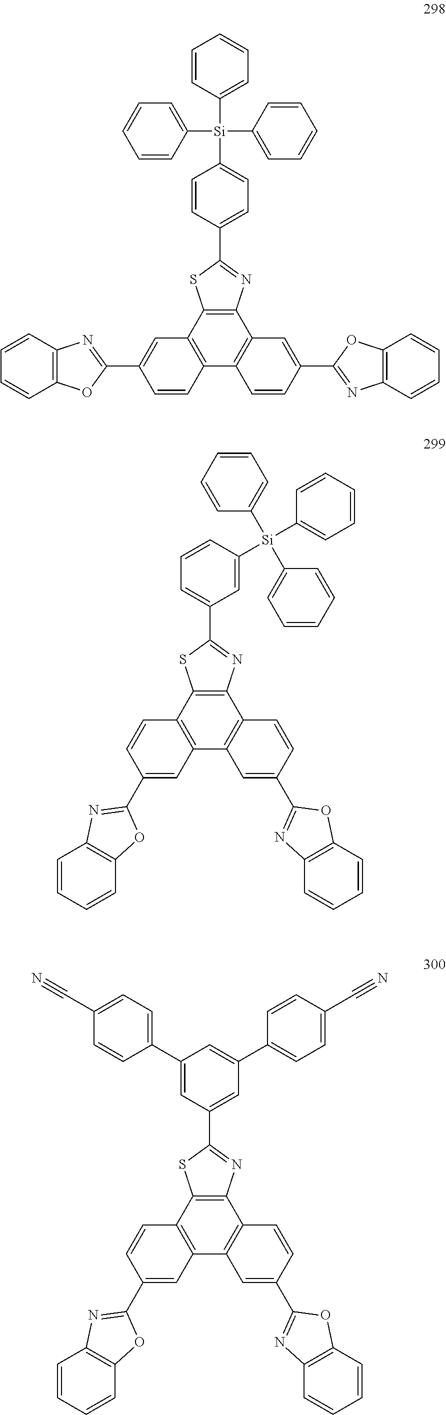 [SMILES: c1ccc([Si](c2ccccc2)(c2ccccc2)c2ccc(-c3nc4c5cc(-c6nc7ccccc7o6)ccc5c5ccc(-c6nc7ccccc7o6)cc5c4s3)cc2)cc1 ; c1ccc([Si](c2ccccc2)(c2ccccc2)c2cccc(-c3nc4c5ccc(-c6nc7ccccc7o6)cc5c5cc(-c6nc7ccccc7o6)ccc5c4s3)c2)cc1 ; N#Cc1ccc(-c2cc(-c3ccc(C#N)cc3)cc(-c3nc4c5ccc(-c6nc7ccccc7o6)cc5c5cc(-c6nc7ccccc7o6)ccc5c4s3)c2)cc1]

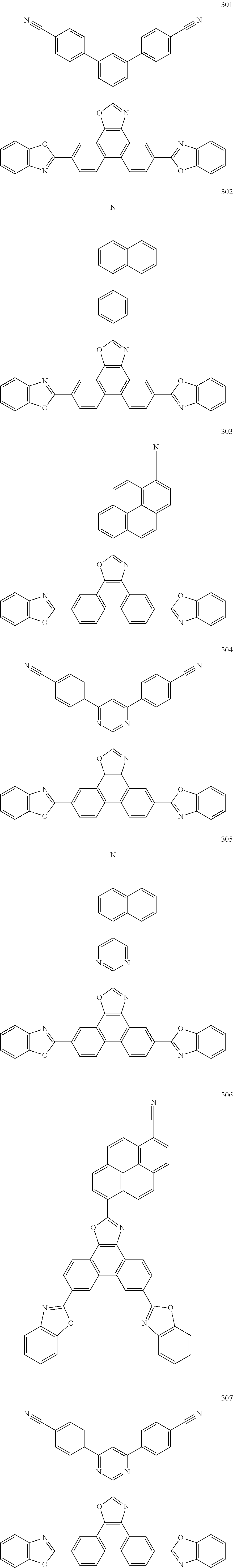

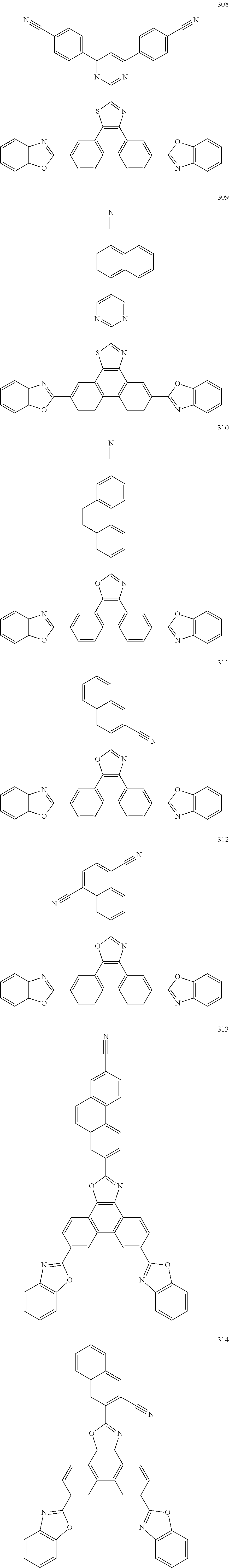

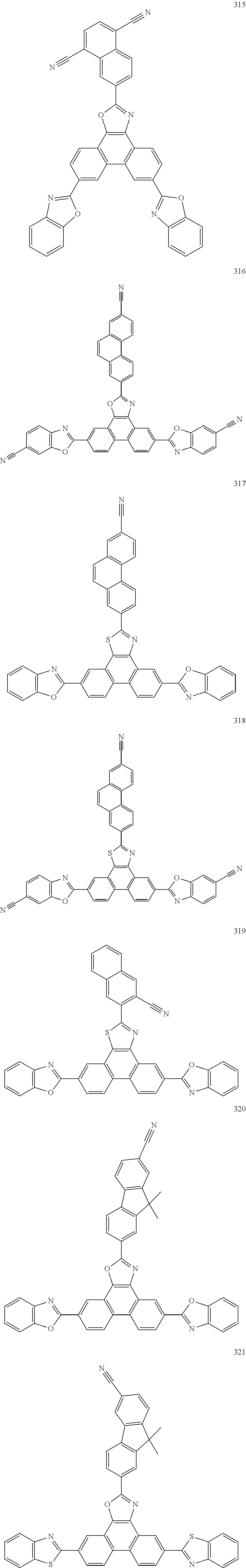

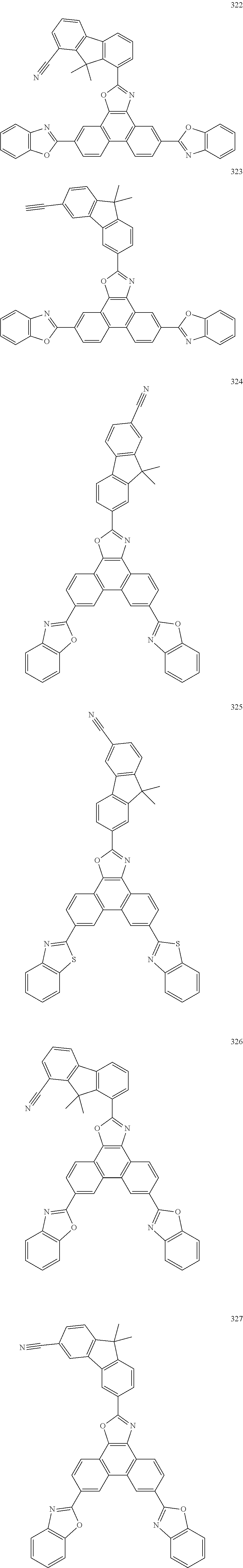

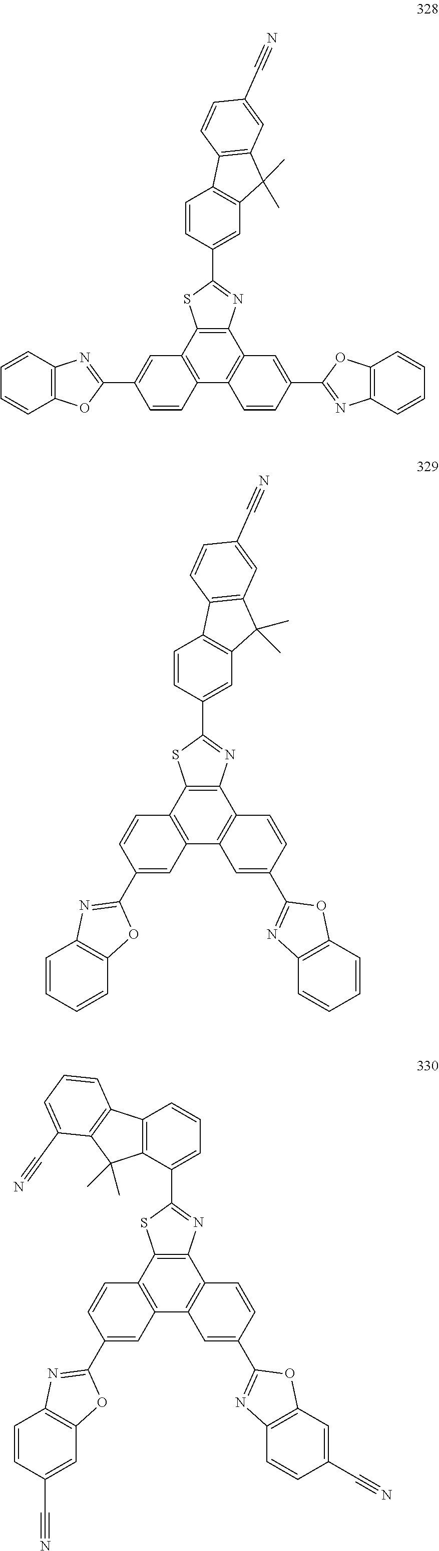

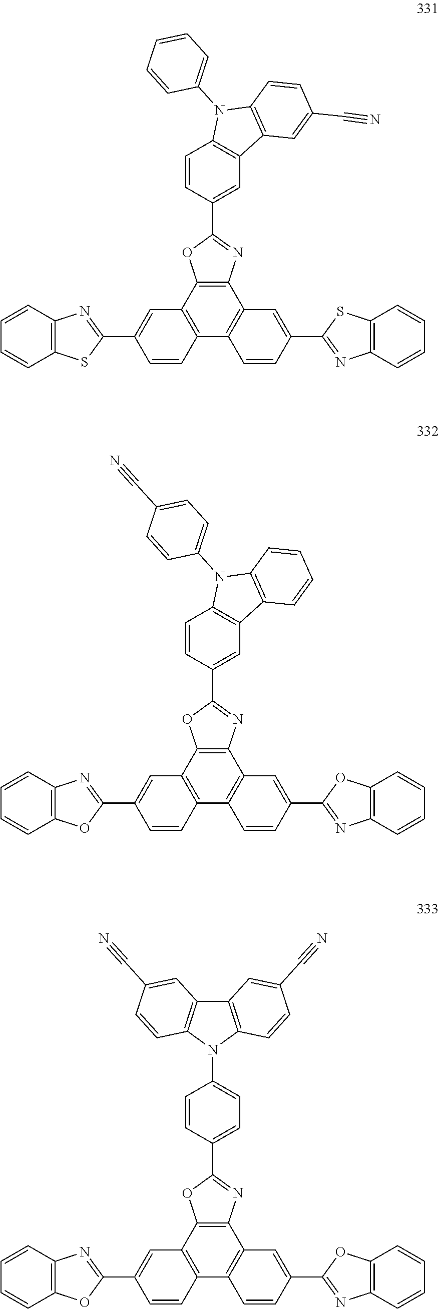 [SMILES: N#Cc1ccc2c(c1)c1cc(-c3nc4c5cc(-c6nc7ccccc7s6)ccc5c5ccc(-c6nc7ccccc7s6)cc5c4o3)ccc1n2-c1ccccc1 ; N#Cc1ccc(-n2c3ccccc3c3cc(-c4nc5c6cc(-c7nc8ccccc8o7)ccc6c6ccc(-c7nc8ccccc8o7)cc6c5o4)ccc32)cc1 ; N#Cc1ccc2c(c1)c1cc(C#N)ccc1n2-c1ccc(-c2nc3c4cc(-c5nc6ccccc6o5)ccc4c4ccc(-c5nc6ccccc6o5)cc4c3o2)cc1]

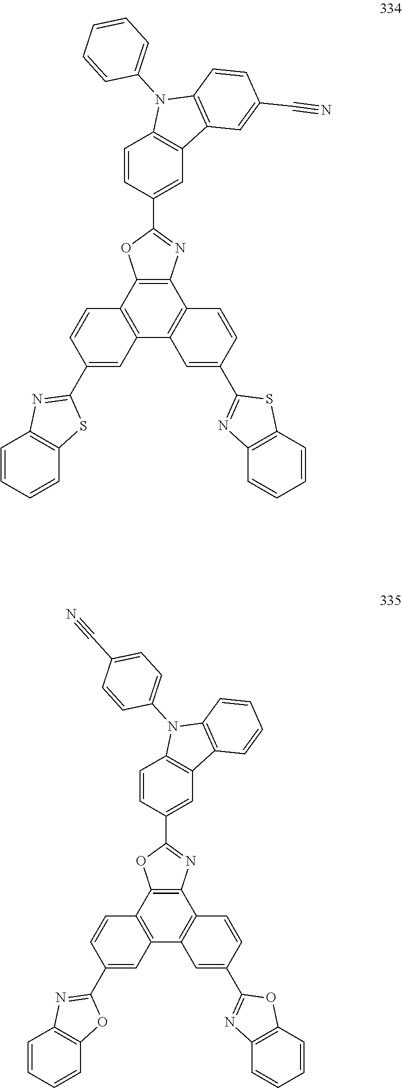 [SMILES: N#Cc1ccc2c(c1)c1cc(-c3nc4c5ccc(-c6nc7ccccc7s6)cc5c5cc(-c6nc7ccccc7s6)ccc5c4o3)ccc1n2-c1ccccc1 ; N#Cc1ccc(-n2c3ccccc3c3cc(-c4nc5c6ccc(-c7nc8ccccc8o7)cc6c6cc(-c7nc8ccccc8o7)ccc6c5o4)ccc32)cc1]

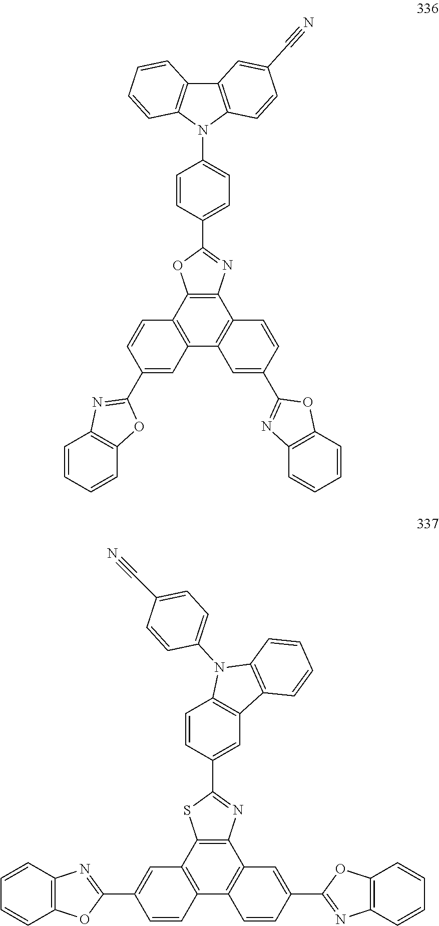 [SMILES: N#Cc1ccc2c(c1)c1ccccc1n2-c1ccc(-c2nc3c4ccc(-c5nc6ccccc6o5)cc4c4cc(-c5nc6ccccc6o5)ccc4c3o2)cc1 ; N#Cc1ccc(-n2c3ccccc3c3cc(-c4nc5c6cc(-c7nc8ccccc8o7)ccc6c6ccc(-c7nc8ccccc8o7)cc6c5s4)ccc32)cc1]

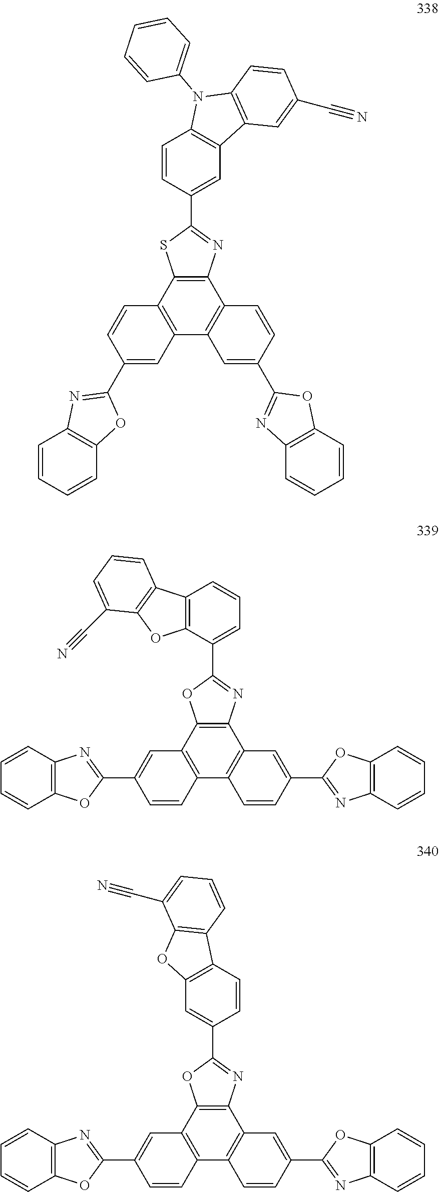 [SMILES: N#Cc1ccc2c(c1)c1cc(-c3nc4c5ccc(-c6nc7ccccc7o6)cc5c5cc(-c6nc7ccccc7o6)ccc5c4s3)ccc1n2-c1ccccc1 ; N#Cc1cccc2c1oc1c(-c3nc4c5cc(-c6nc7ccccc7o6)ccc5c5ccc(-c6nc7ccccc7o6)cc5c4o3)cccc12 ; N#Cc1cccc2c1oc1cc(-c3nc4c5cc(-c6nc7ccccc7o6)ccc5c5ccc(-c6nc7ccccc7o6)cc5c4o3)ccc12]

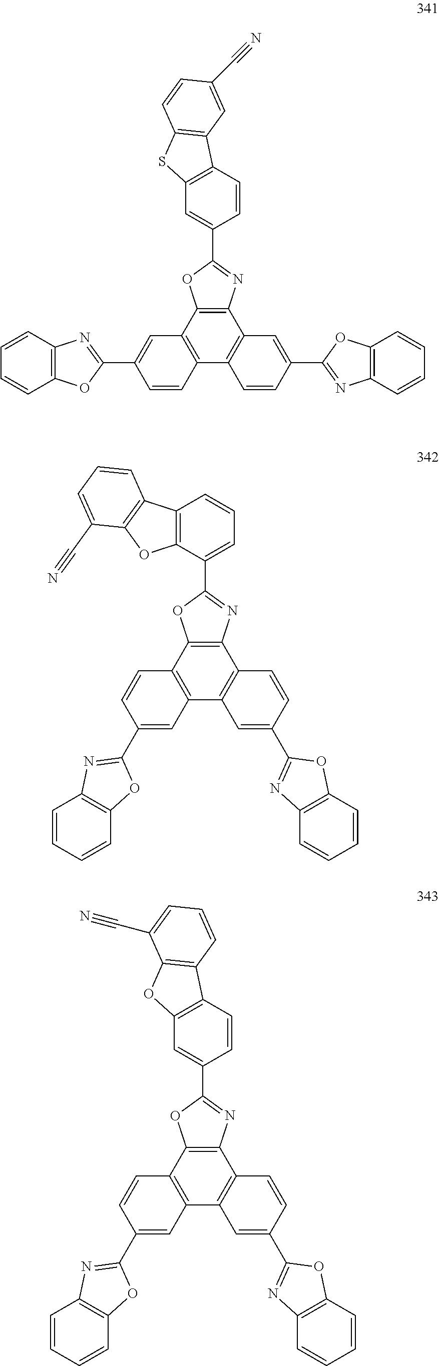 [SMILES: N#Cc1ccc2sc3cc(-c4nc5c6cc(-c7nc8ccccc8o7)ccc6c6ccc(-c7nc8ccccc8o7)cc6c5o4)ccc3c2c1 ; N#Cc1cccc2c1oc1c(-c3nc4c5ccc(-c6nc7ccccc7o6)cc5c5cc(-c6nc7ccccc7o6)ccc5c4o3)cccc12 ; N#Cc1cccc2c1oc1cc(-c3nc4c5ccc(-c6nc7ccccc7o6)cc5c5cc(-c6nc7ccccc7o6)ccc5c4o3)ccc12]

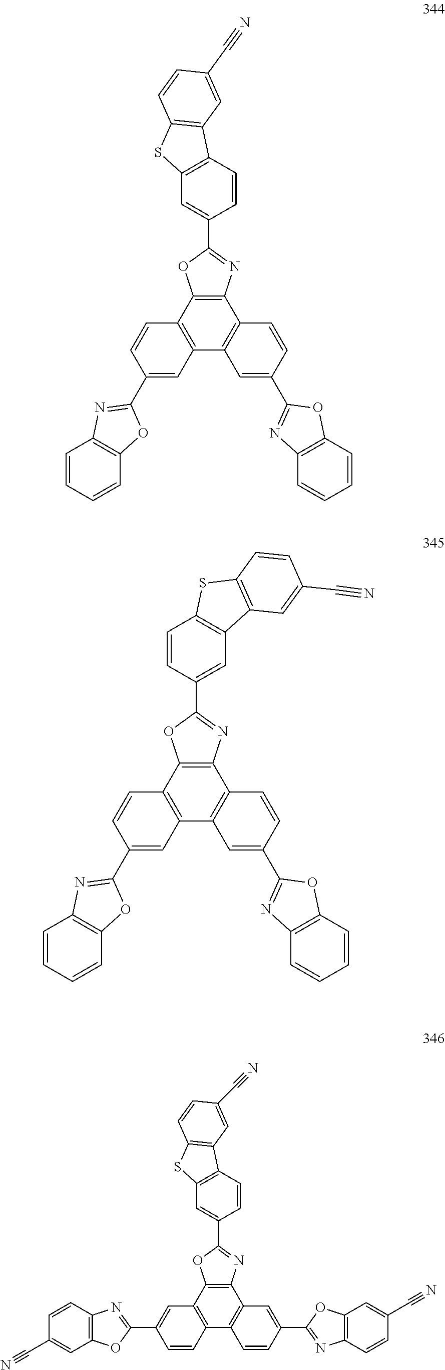 [SMILES: N#Cc1ccc2sc3cc(-c4nc5c6ccc(-c7nc8ccccc8o7)cc6c6cc(-c7nc8ccccc8o7)ccc6c5o4)ccc3c2c1 ; N#Cc1ccc2sc3ccc(-c4nc5c6ccc(-c7nc8ccccc8o7)cc6c6cc(-c7nc8ccccc8o7)ccc6c5o4)cc3c2c1 ; N#Cc1ccc2nc(-c3ccc4c5ccc(-c6nc7ccc(C#N)cc7o6)cc5c5oc(-c6ccc7c(c6)sc6ccc(C#N)cc67)nc5c4c3)oc2c1]

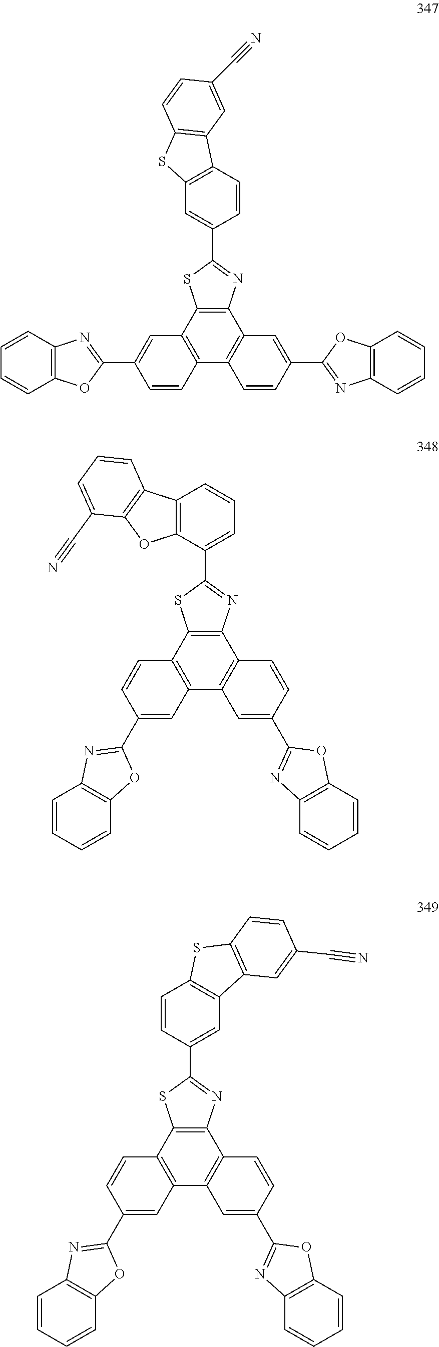 [SMILES: N#Cc1ccc2sc3cc(-c4nc5c6cc(-c7nc8ccccc8o7)ccc6c6ccc(-c7nc8ccccc8o7)cc6c5s4)ccc3c2c1 ; N#Cc1cccc2c1oc1c(-c3nc4c5ccc(-c6nc7ccccc7o6)cc5c5cc(-c6nc7ccccc7o6)ccc5c4s3)cccc12 ; N#Cc1ccc2sc3ccc(-c4nc5c6ccc(-c7nc8ccccc8o7)cc6c6cc(-c7nc8ccccc8o7)ccc6c5s4)cc3c2c1]

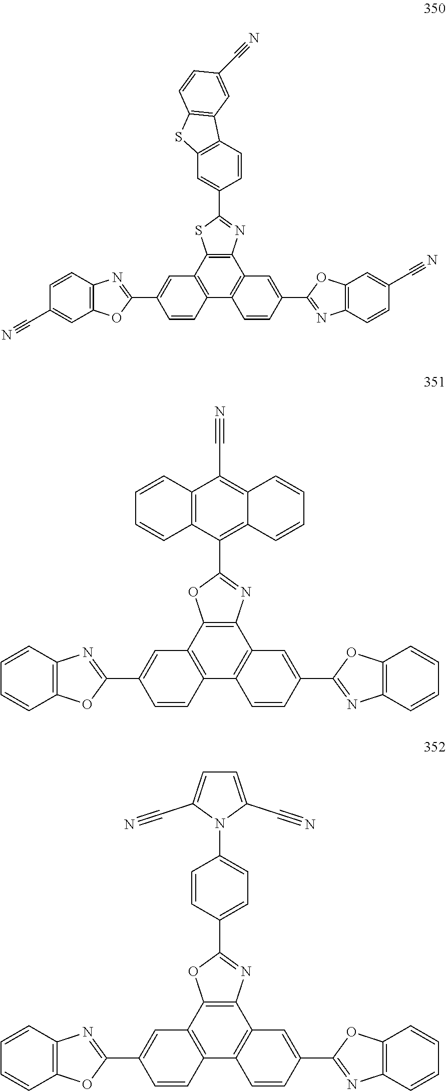 [SMILES: N#Cc1ccc2nc(-c3ccc4c5ccc(-c6nc7ccc(C#N)cc7o6)cc5c5sc(-c6ccc7c(c6)sc6ccc(C#N)cc67)nc5c4c3)oc2c1 ; N#Cc1c2ccccc2c(-c2nc3c4cc(-c5nc6ccccc6o5)ccc4c4ccc(-c5nc6ccccc6o5)cc4c3o2)c2ccccc12 ; N#Cc1ccc(C#N)n1-c1ccc(-c2nc3c4cc(-c5nc6ccccc6o5)ccc4c4ccc(-c5nc6ccccc6o5)cc4c3o2)cc1]

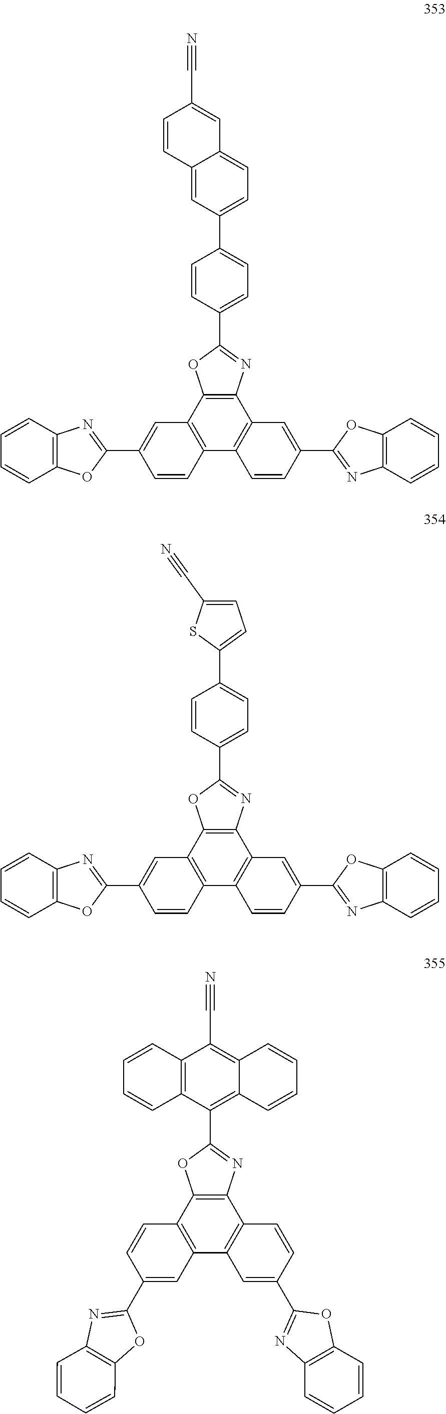 [SMILES: N#Cc1ccc2cc(-c3ccc(-c4nc5c6cc(-c7nc8ccccc8o7)ccc6c6ccc(-c7nc8ccccc8o7)cc6c5o4)cc3)ccc2c1 ; N#Cc1ccc(-c2ccc(-c3nc4c5cc(-c6nc7ccccc7o6)ccc5c5ccc(-c6nc7ccccc7o6)cc5c4o3)cc2)s1 ; N#Cc1c2ccccc2c(-c2nc3c4ccc(-c5nc6ccccc6o5)cc4c4cc(-c5nc6ccccc6o5)ccc4c3o2)c2ccccc12]

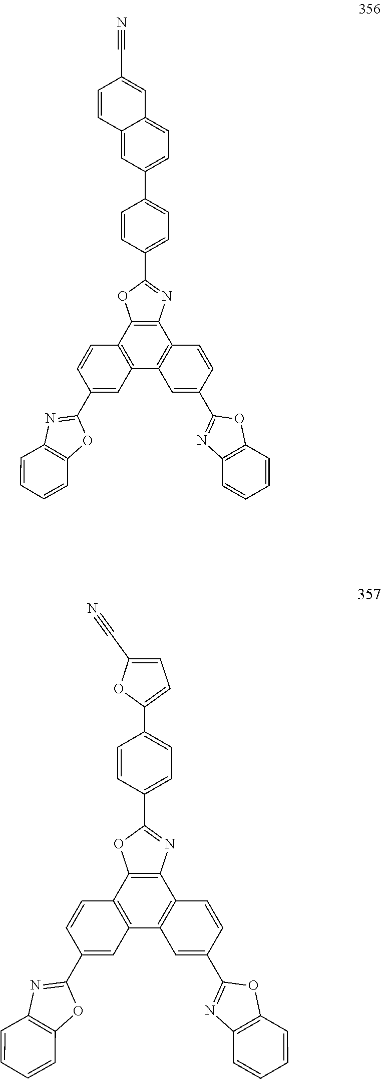 [SMILES: N#Cc1ccc2cc(-c3ccc(-c4nc5c6ccc(-c7nc8ccccc8o7)cc6c6cc(-c7nc8ccccc8o7)ccc6c5o4)cc3)ccc2c1 ; N#Cc1ccc(-c2ccc(-c3nc4c5ccc(-c6nc7ccccc7o6)cc5c5cc(-c6nc7ccccc7o6)ccc5c4o3)cc2)o1]

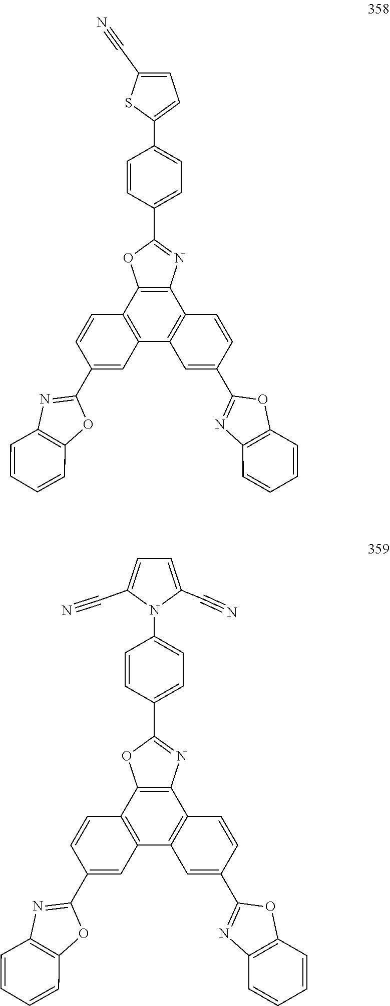 [SMILES: N#Cc1ccc(-c2ccc(-c3nc4c5ccc(-c6nc7ccccc7o6)cc5c5cc(-c6nc7ccccc7o6)ccc5c4o3)cc2)s1 ; N#Cc1ccc(C#N)n1-c1ccc(-c2nc3c4ccc(-c5nc6ccccc6o5)cc4c4cc(-c5nc6ccccc6o5)ccc4c3o2)cc1]

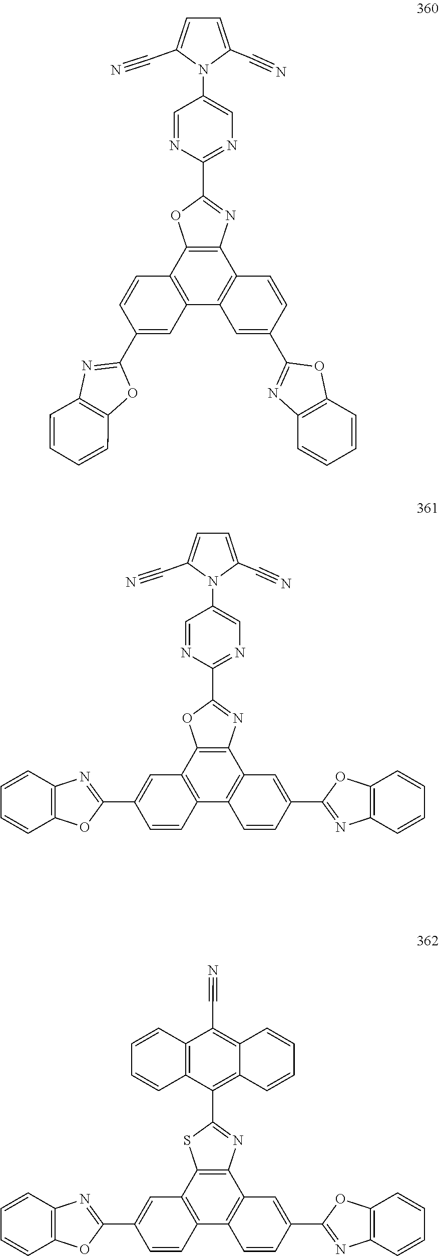 [SMILES: N#Cc1ccc(C#N)n1-c1cnc(-c2nc3c4ccc(-c5nc6ccccc6o5)cc4c4cc(-c5nc6ccccc6o5)ccc4c3o2)nc1 ; N#Cc1ccc(C#N)n1-c1cnc(-c2nc3c4cc(-c5nc6ccccc6o5)ccc4c4ccc(-c5nc6ccccc6o5)cc4c3o2)nc1 ; N#Cc1c2ccccc2c(-c2nc3c4cc(-c5nc6ccccc6o5)ccc4c4ccc(-c5nc6ccccc6o5)cc4c3s2)c2ccccc12]

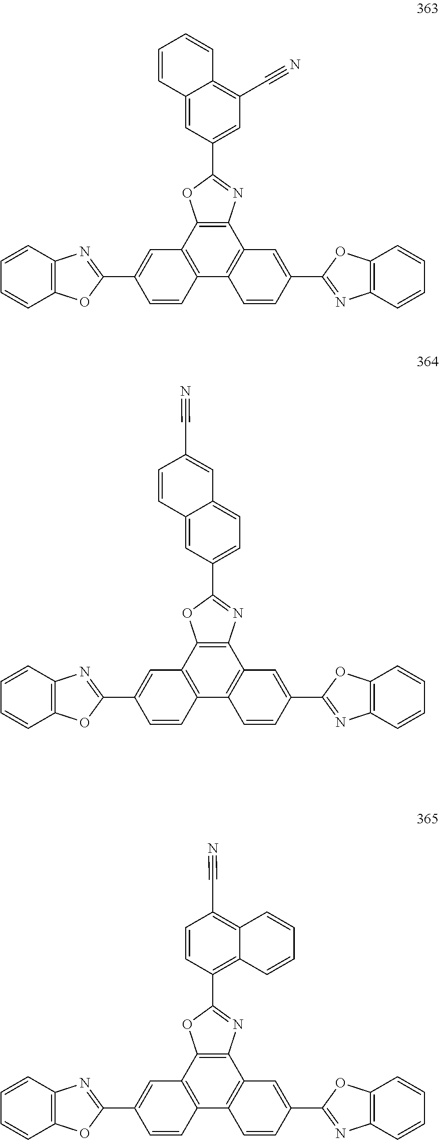 [SMILES: N#Cc1cc(-c2nc3c4cc(-c5nc6ccccc6o5)ccc4c4ccc(-c5nc6ccccc6o5)cc4c3o2)cc2ccccc12 ; N#Cc1ccc2cc(-c3nc4c5cc(-c6nc7ccccc7o6)ccc5c5ccc(-c6nc7ccccc7o6)cc5c4o3)ccc2c1 ; N#Cc1ccc(-c2nc3c4cc(-c5nc6ccccc6o5)ccc4c4ccc(-c5nc6ccccc6o5)cc4c3o2)c2ccccc12]

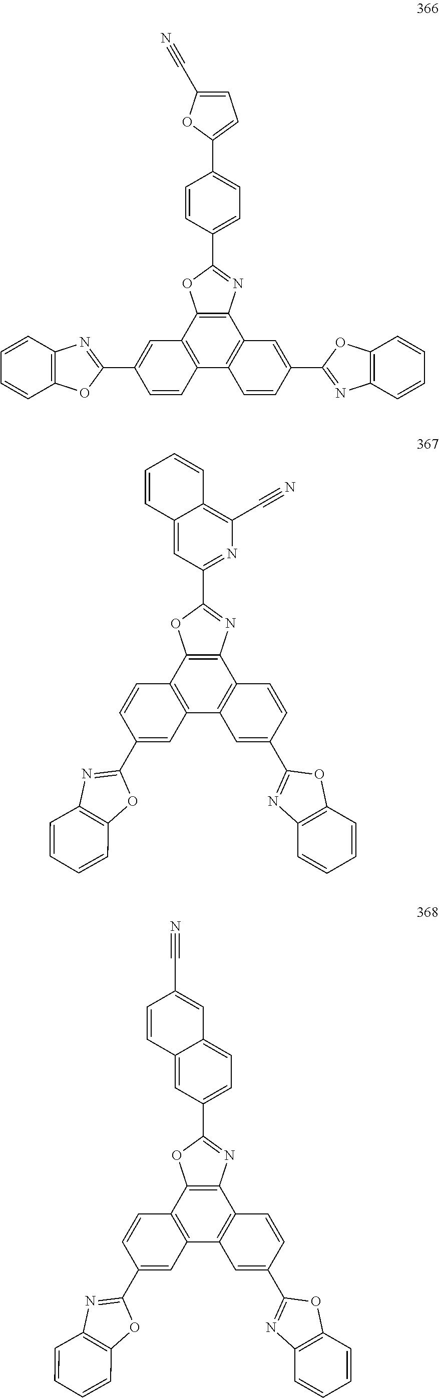 [SMILES: N#Cc1ccc(-c2ccc(-c3nc4c5cc(-c6nc7ccccc7o6)ccc5c5ccc(-c6nc7ccccc7o6)cc5c4o3)cc2)o1 ; N#Cc1nc(-c2nc3c4ccc(-c5nc6ccccc6o5)cc4c4cc(-c5nc6ccccc6o5)ccc4c3o2)cc2ccccc12 ; N#Cc1ccc2cc(-c3nc4c5ccc(-c6nc7ccccc7o6)cc5c5cc(-c6nc7ccccc7o6)ccc5c4o3)ccc2c1]

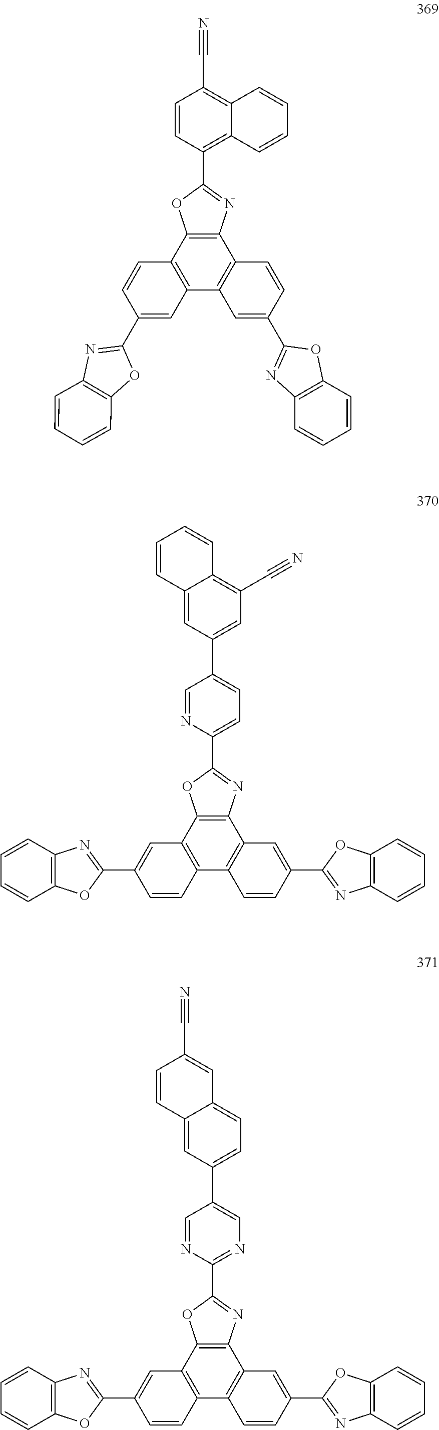 [SMILES: N#Cc1ccc(-c2nc3c4ccc(-c5nc6ccccc6o5)cc4c4cc(-c5nc6ccccc6o5)ccc4c3o2)c2ccccc12 ; N#Cc1cc(-c2ccc(-c3nc4c5cc(-c6nc7ccccc7o6)ccc5c5ccc(-c6nc7ccccc7o6)cc5c4o3)nc2)cc2ccccc12 ; N#Cc1ccc2cc(-c3cnc(-c4nc5c6cc(-c7nc8ccccc8o7)ccc6c6ccc(-c7nc8ccccc8o7)cc6c5o4)nc3)ccc2c1]

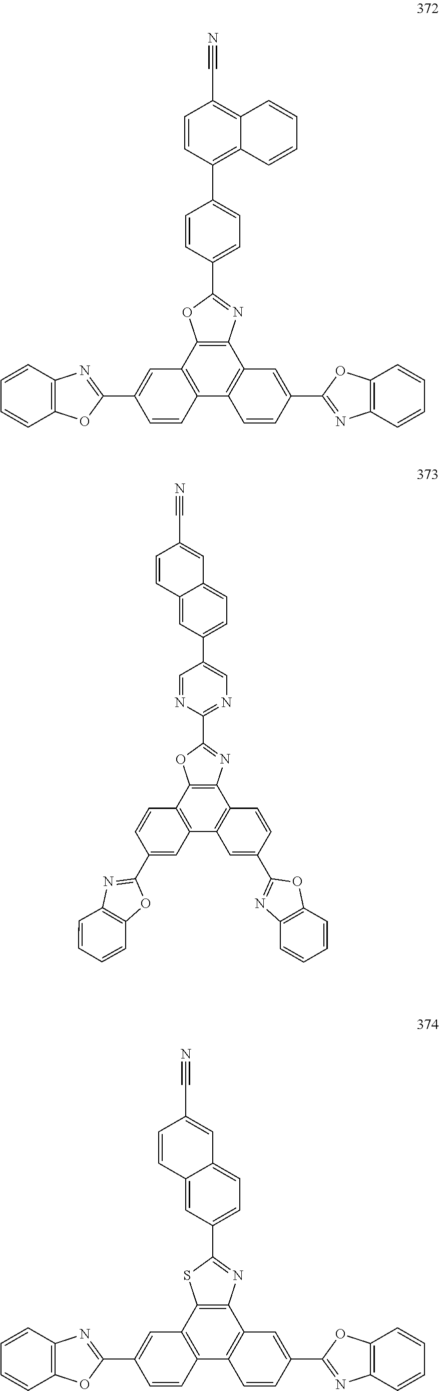 [SMILES: N#Cc1ccc(-c2ccc(-c3nc4c5cc(-c6nc7ccccc7o6)ccc5c5ccc(-c6nc7ccccc7o6)cc5c4o3)cc2)c2ccccc12 ; N#Cc1ccc2cc(-c3cnc(-c4nc5c6ccc(-c7nc8ccccc8o7)cc6c6cc(-c7nc8ccccc8o7)ccc6c5o4)nc3)ccc2c1 ; N#Cc1ccc2cc(-c3nc4c5cc(-c6nc7ccccc7o6)ccc5c5ccc(-c6nc7ccccc7o6)cc5c4s3)ccc2c1]

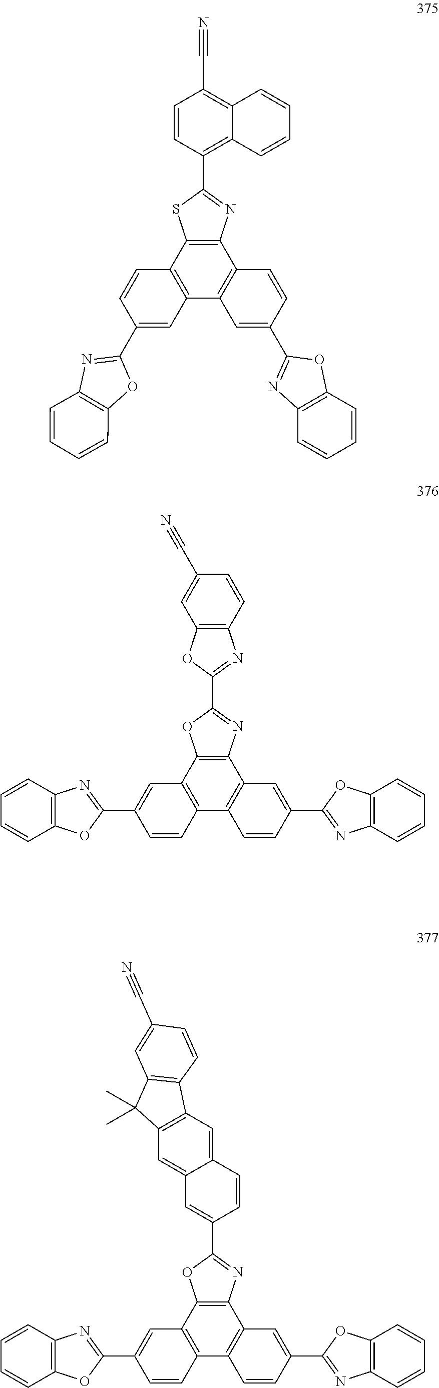 [SMILES: N#Cc1ccc(-c2nc3c4ccc(-c5nc6ccccc6o5)cc4c4cc(-c5nc6ccccc6o5)ccc4c3s2)c2ccccc12 ; N#Cc1ccc2nc(-c3nc4c5cc(-c6nc7ccccc7o6)ccc5c5ccc(-c6nc7ccccc7o6)cc5c4o3)oc2c1 ; CC1(C)c2cc(C#N)ccc2-c2cc3ccc(-c4nc5c6cc(-c7nc8ccccc8o7)ccc6c6ccc(-c7nc8ccccc8o7)cc6c5o4)cc3cc21]

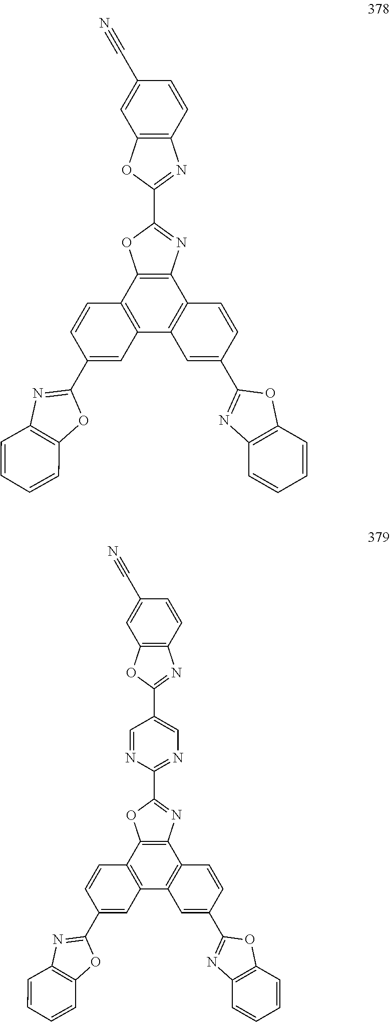 [SMILES: N#Cc1ccc2nc(-c3nc4c5ccc(-c6nc7ccccc7o6)cc5c5cc(-c6nc7ccccc7o6)ccc5c4o3)oc2c1 ; N#Cc1ccc2nc(-c3cnc(-c4nc5c6ccc(-c7nc8ccccc8o7)cc6c6cc(-c7nc8ccccc8o7)ccc6c5o4)nc3)oc2c1]

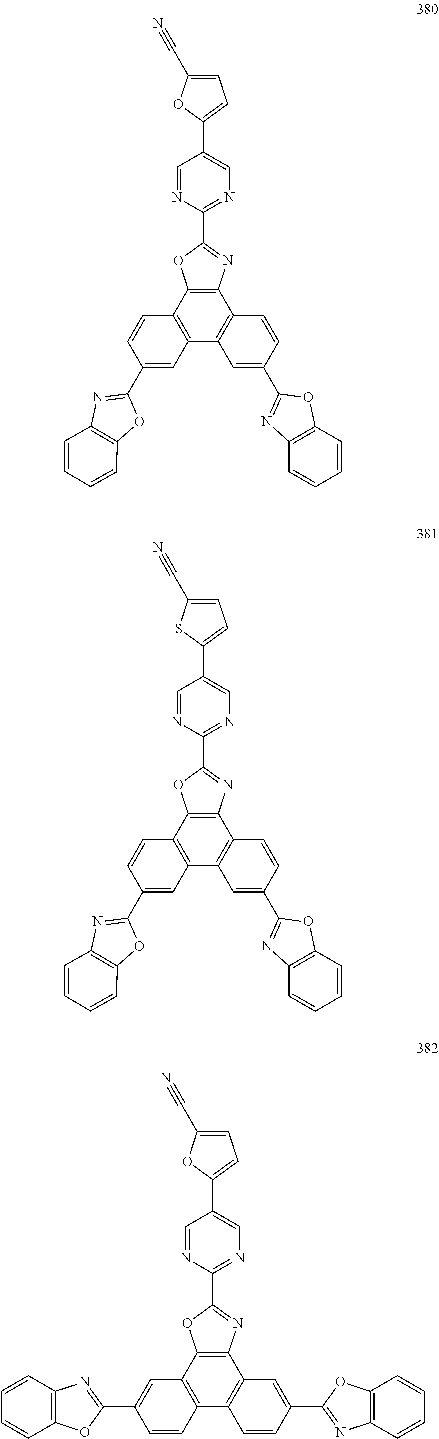 [SMILES: N#Cc1ccc(-c2cnc(-c3nc4c5ccc(-c6nc7ccccc7o6)cc5c5cc(-c6nc7ccccc7o6)ccc5c4o3)nc2)o1 ; N#Cc1ccc(-c2cnc(-c3nc4c5ccc(-c6nc7ccccc7o6)cc5c5cc(-c6nc7ccccc7o6)ccc5c4o3)nc2)s1 ; N#Cc1ccc(-c2cnc(-c3nc4c5cc(-c6nc7ccccc7o6)ccc5c5ccc(-c6nc7ccccc7o6)cc5c4o3)nc2)o1]

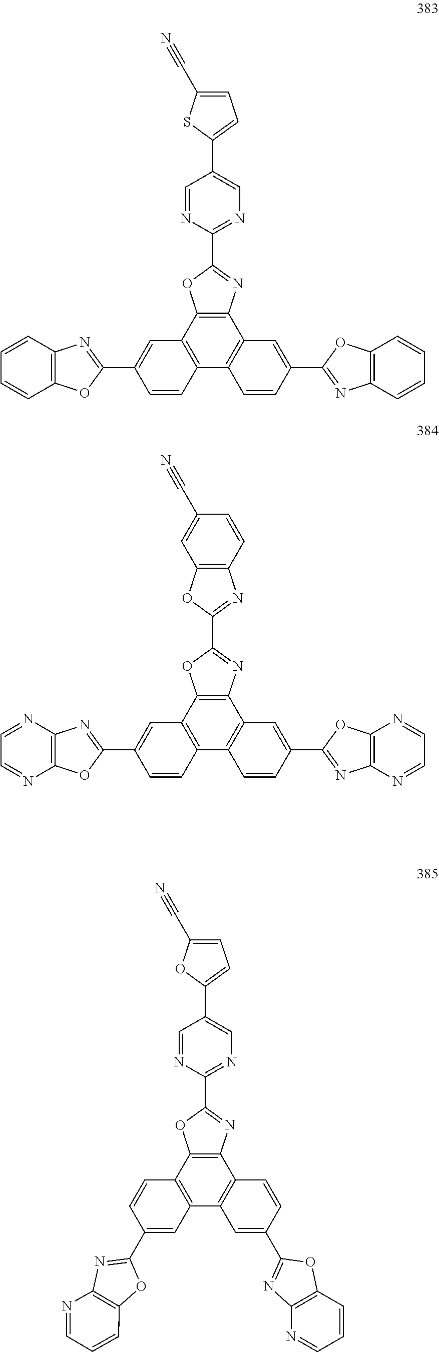 [SMILES: N#Cc1ccc(-c2cnc(-c3nc4c5cc(-c6nc7ccccc7o6)ccc5c5ccc(-c6nc7ccccc7o6)cc5c4o3)nc2)s1 ; N#Cc1ccc2nc(-c3nc4c5cc(-c6nc7nccnc7o6)ccc5c5ccc(-c6nc7nccnc7o6)cc5c4o3)oc2c1 ; N#Cc1ccc(-c2cnc(-c3nc4c5ccc(-c6nc7ncccc7o6)cc5c5cc(-c6nc7ncccc7o6)ccc5c4o3)nc2)o1]

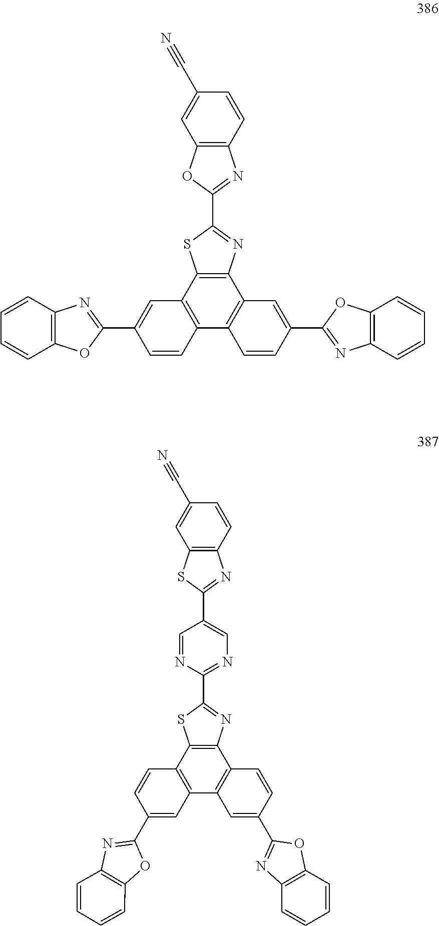 [SMILES: N#Cc1ccc2nc(-c3nc4c5cc(-c6nc7ccccc7o6)ccc5c5ccc(-c6nc7ccccc7o6)cc5c4s3)oc2c1 ; N#Cc1ccc2nc(-c3cnc(-c4nc5c6ccc(-c7nc8ccccc8o7)cc6c6cc(-c7nc8ccccc8o7)ccc6c5s4)nc3)sc2c1]

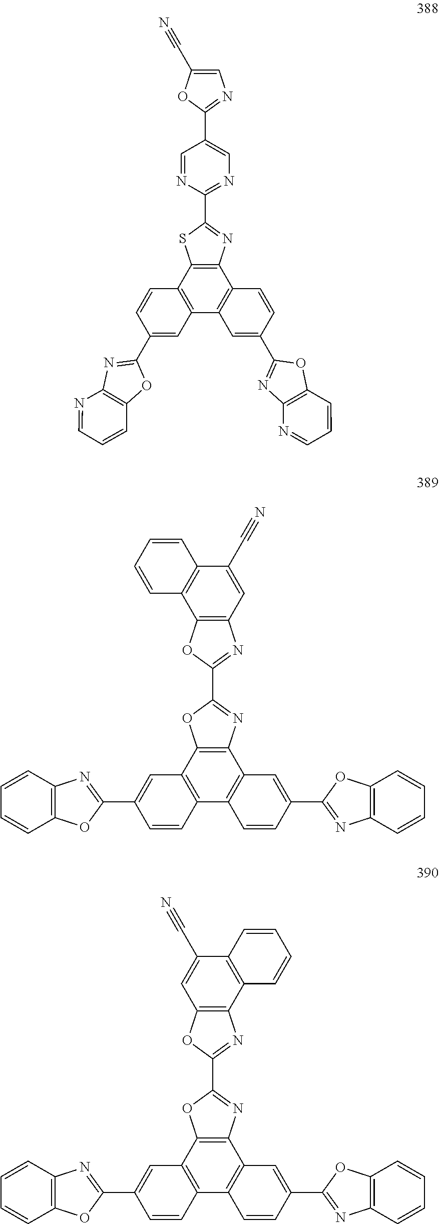 [SMILES: N#Cc1cnc(-c2cnc(-c3nc4c5ccc(-c6nc7ncccc7o6)cc5c5cc(-c6nc7ncccc7o6)ccc5c4s3)nc2)o1 ; N#Cc1cc2nc(-c3nc4c5cc(-c6nc7ccccc7o6)ccc5c5ccc(-c6nc7ccccc7o6)cc5c4o3)oc2c2ccccc12 ; N#Cc1cc2oc(-c3nc4c5cc(-c6nc7ccccc7o6)ccc5c5ccc(-c6nc7ccccc7o6)cc5c4o3)nc2c2ccccc12]

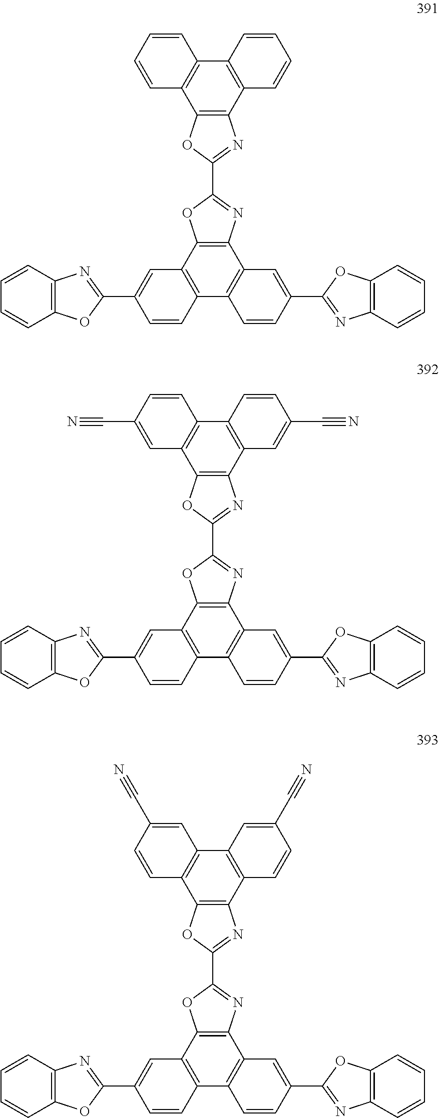 [SMILES: c1ccc2oc(-c3ccc4c5ccc(-c6nc7ccccc7o6)cc5c5oc(-c6nc7c8ccccc8c8ccccc8c7o6)nc5c4c3)nc2c1 ; N#Cc1ccc2c3ccc(C#N)cc3c3oc(-c4nc5c6cc(-c7nc8ccccc8o7)ccc6c6ccc(-c7nc8ccccc8o7)cc6c5o4)nc3c2c1 ; N#Cc1ccc2c(c1)c1cc(C#N)ccc1c1oc(-c3nc4c5cc(-c6nc7ccccc7o6)ccc5c5ccc(-c6nc7ccccc7o6)cc5c4o3)nc21]

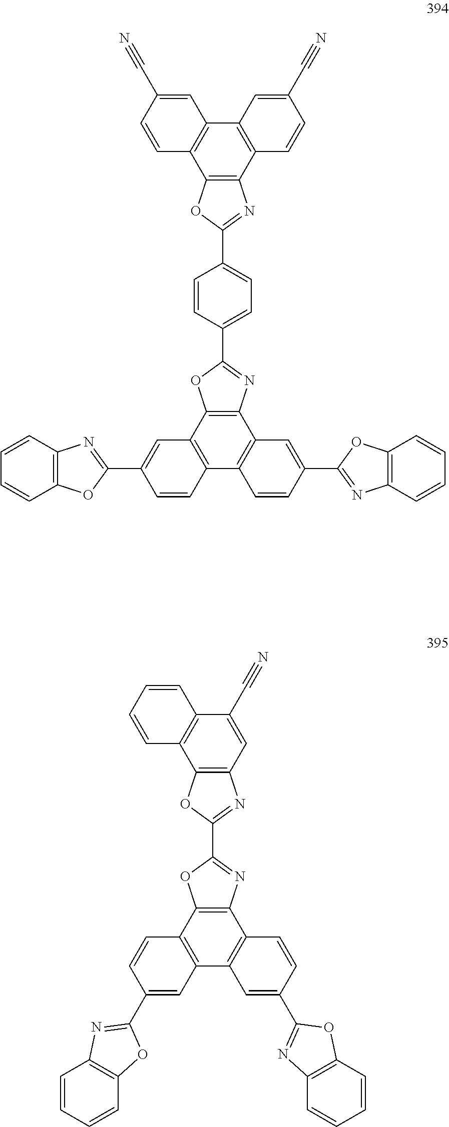 [SMILES: N#Cc1ccc2c(c1)c1cc(C#N)ccc1c1oc(-c3ccc(-c4nc5c6cc(-c7nc8ccccc8o7)ccc6c6ccc(-c7nc8ccccc8o7)cc6c5o4)cc3)nc21 ; N#Cc1cc2nc(-c3nc4c5ccc(-c6nc7ccccc7o6)cc5c5cc(-c6nc7ccccc7o6)ccc5c4o3)oc2c2ccccc12]

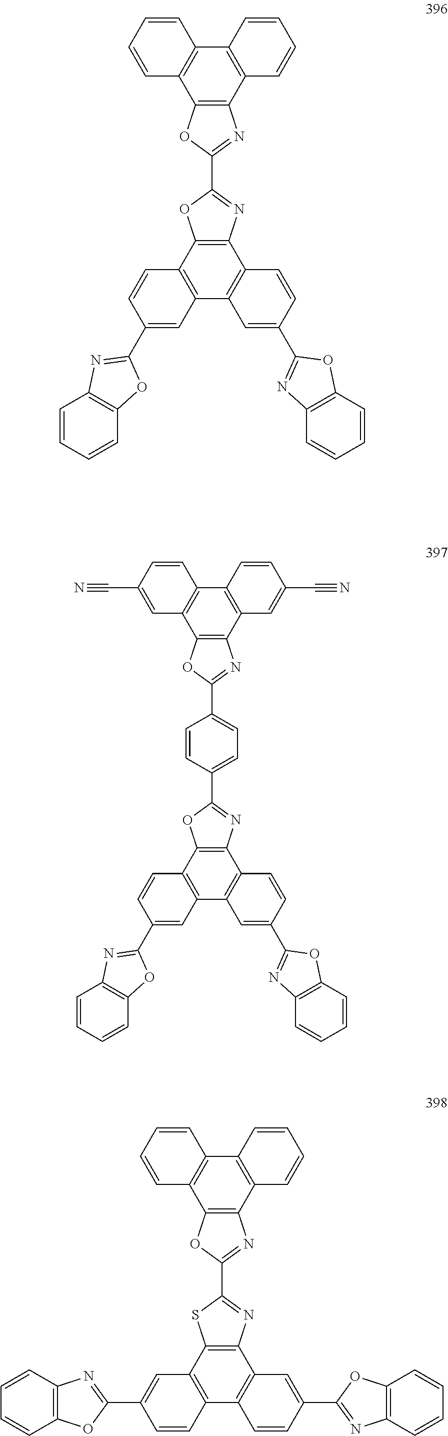 [SMILES: c1ccc2oc(-c3ccc4c(c3)c3cc(-c5nc6ccccc6o5)ccc3c3oc(-c5nc6c7ccccc7c7ccccc7c6o5)nc43)nc2c1 ; N#Cc1ccc2c3ccc(C#N)cc3c3oc(-c4ccc(-c5nc6c7ccc(-c8nc9ccccc9o8)cc7c7cc(-c8nc9ccccc9o8)ccc7c6o5)cc4)nc3c2c1 ; c1ccc2oc(-c3ccc4c5ccc(-c6nc7ccccc7o6)cc5c5sc(-c6nc7c8ccccc8c8ccccc8c7o6)nc5c4c3)nc2c1]

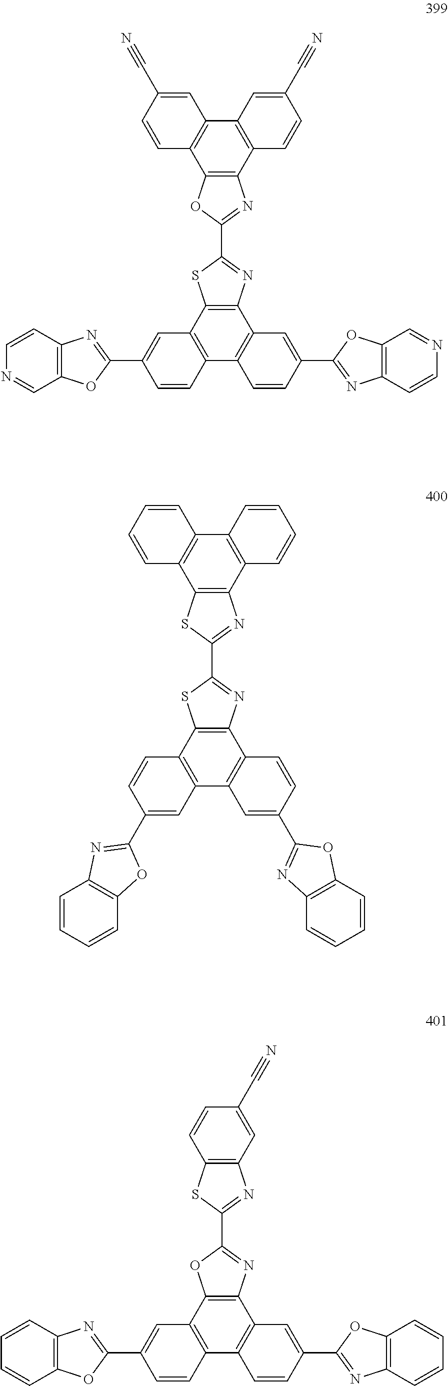 [SMILES: N#Cc1ccc2c(c1)c1cc(C#N)ccc1c1oc(-c3nc4c5cc(-c6nc7ccncc7o6)ccc5c5ccc(-c6nc7ccncc7o6)cc5c4s3)nc21 ; c1ccc2oc(-c3ccc4c(c3)c3cc(-c5nc6ccccc6o5)ccc3c3sc(-c5nc6c7ccccc7c7ccccc7c6s5)nc43)nc2c1 ; N#Cc1ccc2sc(-c3nc4c5cc(-c6nc7ccccc7o6)ccc5c5ccc(-c6nc7ccccc7o6)cc5c4o3)nc2c1]

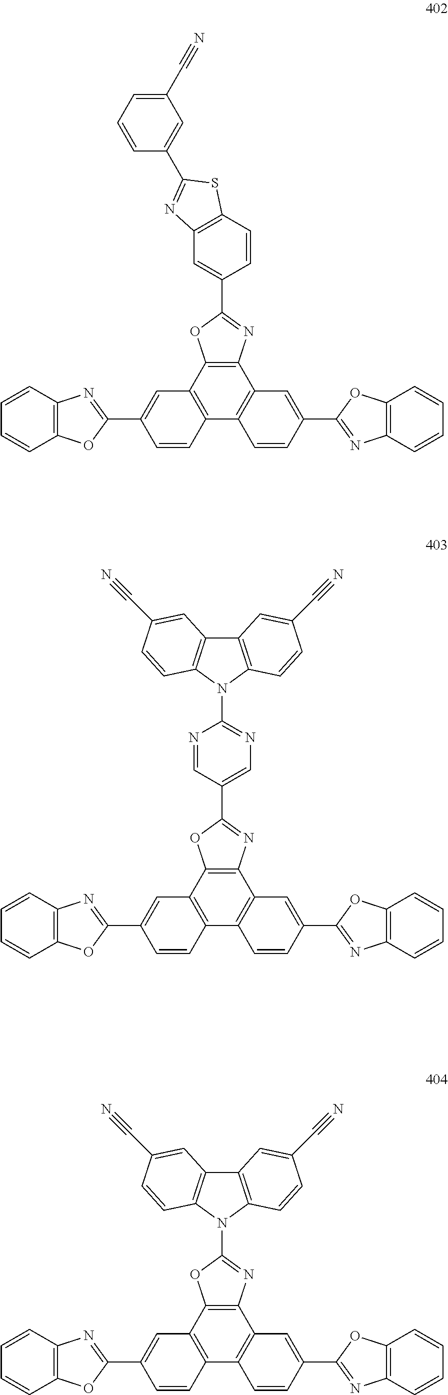 [SMILES: N#Cc1cccc(-c2nc3cc(-c4nc5c6cc(-c7nc8ccccc8o7)ccc6c6ccc(-c7nc8ccccc8o7)cc6c5o4)ccc3s2)c1 ; N#Cc1ccc2c(c1)c1cc(C#N)ccc1n2-c1ncc(-c2nc3c4cc(-c5nc6ccccc6o5)ccc4c4ccc(-c5nc6ccccc6o5)cc4c3o2)cn1 ; N#Cc1ccc2c(c1)c1cc(C#N)ccc1n2-c1nc2c3cc(-c4nc5ccccc5o4)ccc3c3ccc(-c4nc5ccccc5o4)cc3c2o1]

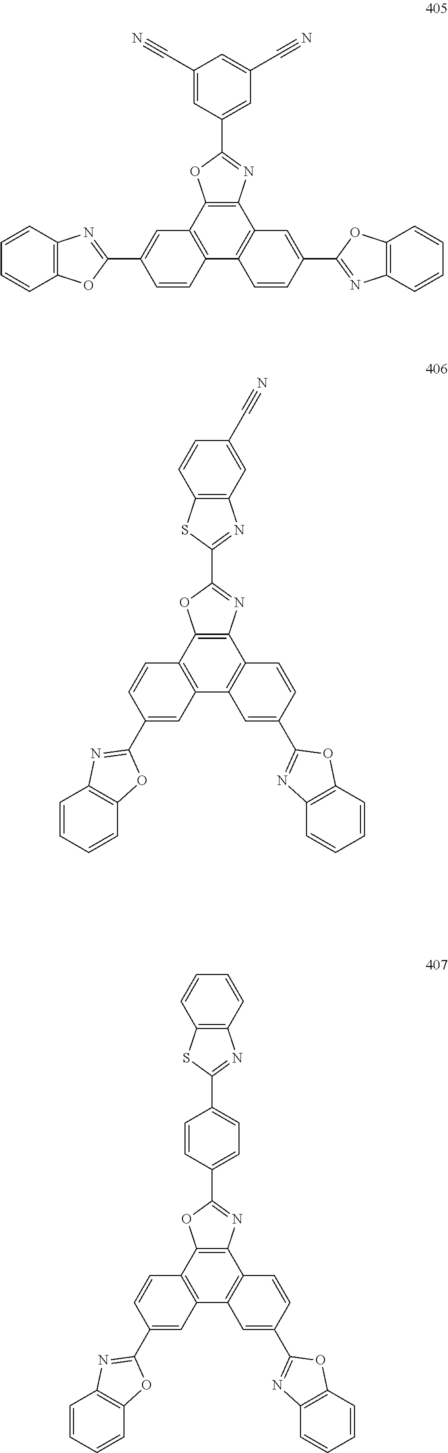 [SMILES: N#Cc1cc(C#N)cc(-c2nc3c4cc(-c5nc6ccccc6o5)ccc4c4ccc(-c5nc6ccccc6o5)cc4c3o2)c1 ; N#Cc1ccc2sc(-c3nc4c5ccc(-c6nc7ccccc7o6)cc5c5cc(-c6nc7ccccc7o6)ccc5c4o3)nc2c1 ; c1ccc2oc(-c3ccc4c(c3)c3cc(-c5nc6ccccc6o5)ccc3c3oc(-c5ccc(-c6nc7ccccc7s6)cc5)nc43)nc2c1]

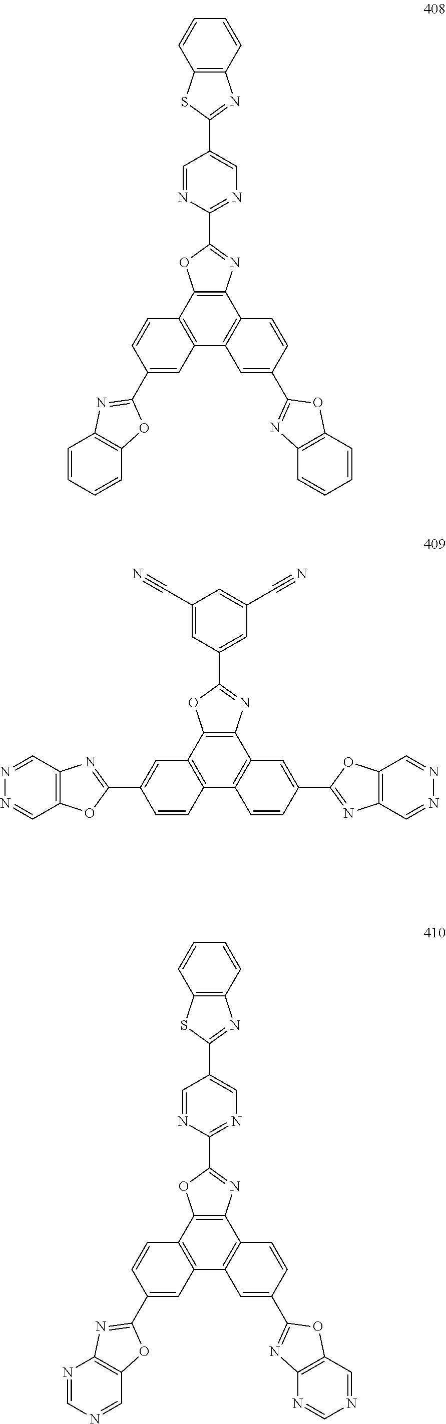 [SMILES: c1ccc2oc(-c3ccc4c(c3)c3cc(-c5nc6ccccc6o5)ccc3c3oc(-c5ncc(-c6nc7ccccc7s6)cn5)nc43)nc2c1 ; N#Cc1cc(C#N)cc(-c2nc3c4cc(-c5nc6cnncc6o5)ccc4c4ccc(-c5nc6cnncc6o5)cc4c3o2)c1 ; c1ccc2sc(-c3cnc(-c4nc5c6ccc(-c7nc8ncncc8o7)cc6c6cc(-c7nc8ncncc8o7)ccc6c5o4)nc3)nc2c1]

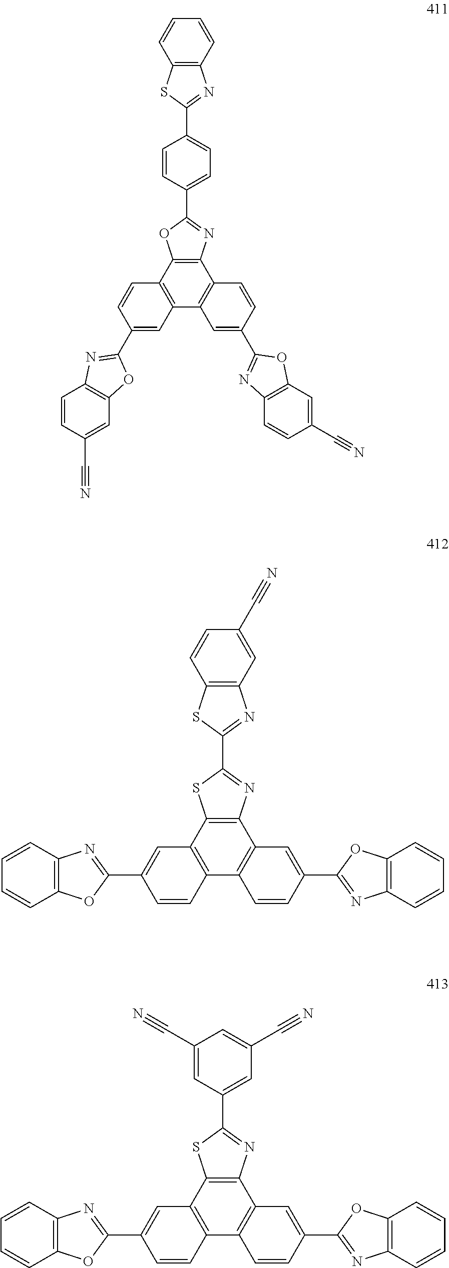 [SMILES: N#Cc1ccc2nc(-c3ccc4c(c3)c3cc(-c5nc6ccc(C#N)cc6o5)ccc3c3oc(-c5ccc(-c6nc7ccccc7s6)cc5)nc43)oc2c1 ; N#Cc1ccc2sc(-c3nc4c5cc(-c6nc7ccccc7o6)ccc5c5ccc(-c6nc7ccccc7o6)cc5c4s3)nc2c1 ; N#Cc1cc(C#N)cc(-c2nc3c4cc(-c5nc6ccccc6o5)ccc4c4ccc(-c5nc6ccccc6o5)cc4c3s2)c1]

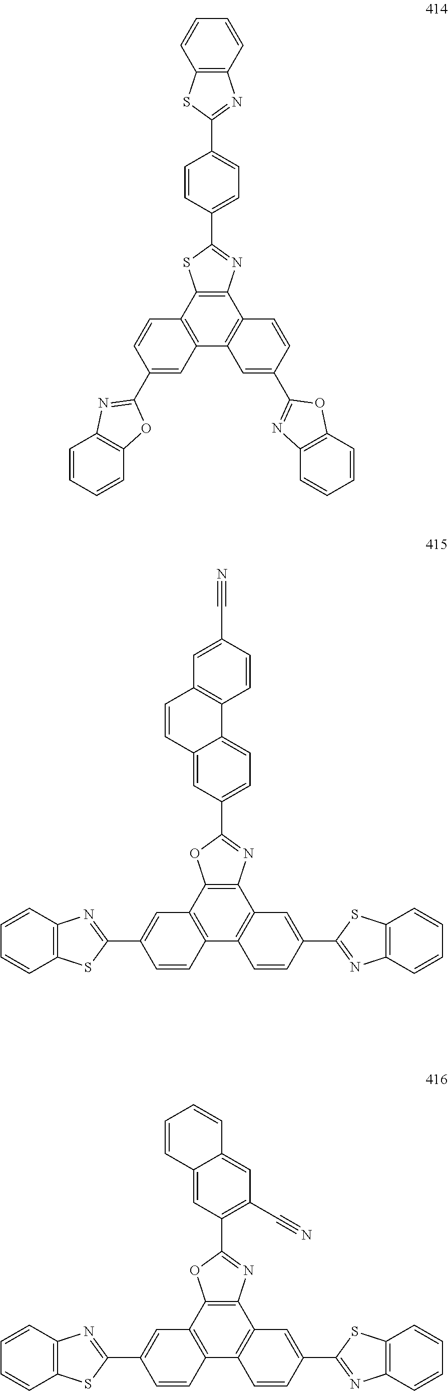 [SMILES: c1ccc2oc(-c3ccc4c(c3)c3cc(-c5nc6ccccc6o5)ccc3c3sc(-c5ccc(-c6nc7ccccc7s6)cc5)nc43)nc2c1 ; N#Cc1ccc2c(ccc3cc(-c4nc5c6cc(-c7nc8ccccc8s7)ccc6c6ccc(-c7nc8ccccc8s7)cc6c5o4)ccc32)c1 ; N#Cc1cc2ccccc2cc1-c1nc2c3cc(-c4nc5ccccc5s4)ccc3c3ccc(-c4nc5ccccc5s4)cc3c2o1]

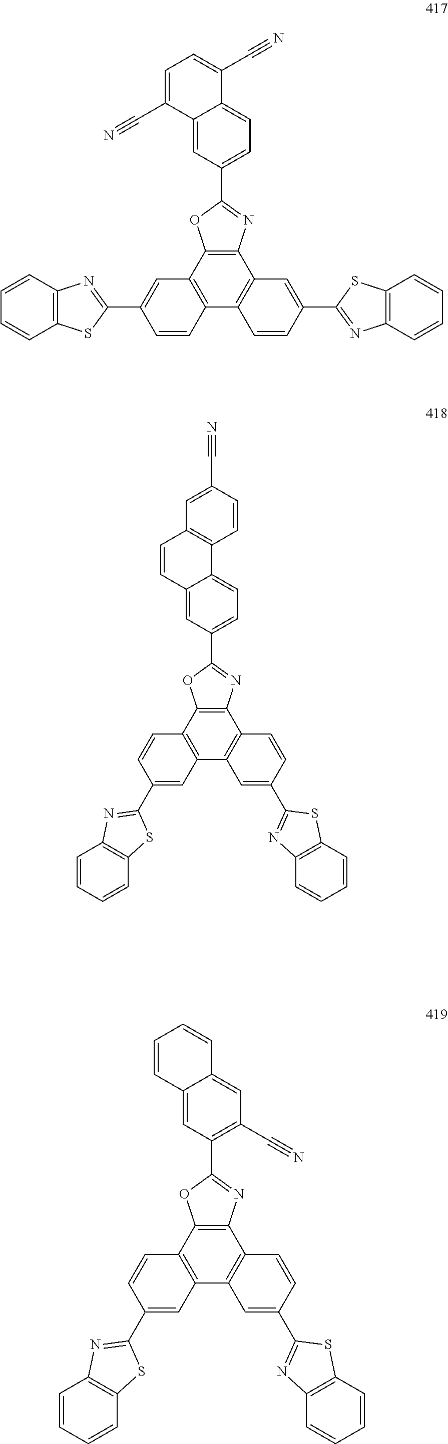 [SMILES: N#Cc1ccc(C#N)c2cc(-c3nc4c5cc(-c6nc7ccccc7s6)ccc5c5ccc(-c6nc7ccccc7s6)cc5c4o3)ccc12 ; N#Cc1ccc2c(ccc3cc(-c4nc5c6ccc(-c7nc8ccccc8s7)cc6c6cc(-c7nc8ccccc8s7)ccc6c5o4)ccc32)c1 ; N#Cc1cc2ccccc2cc1-c1nc2c3ccc(-c4nc5ccccc5s4)cc3c3cc(-c4nc5ccccc5s4)ccc3c2o1]

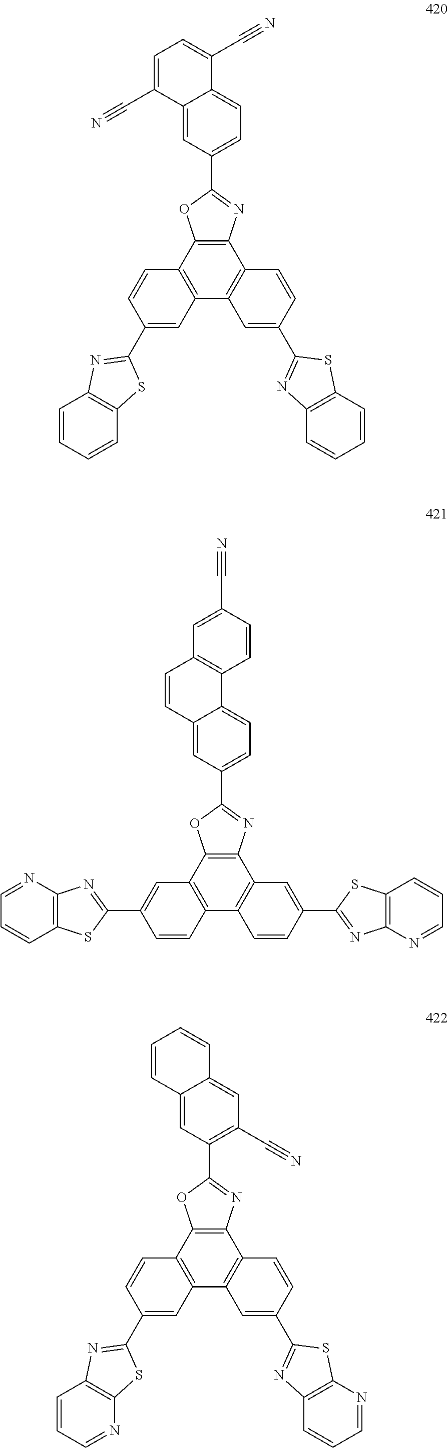 [SMILES: N#Cc1ccc(C#N)c2cc(-c3nc4c5ccc(-c6nc7ccccc7s6)cc5c5cc(-c6nc7ccccc7s6)ccc5c4o3)ccc12 ; N#Cc1ccc2c(ccc3cc(-c4nc5c6cc(-c7nc8ncccc8s7)ccc6c6ccc(-c7nc8ncccc8s7)cc6c5o4)ccc32)c1 ; N#Cc1cc2ccccc2cc1-c1nc2c3ccc(-c4nc5cccnc5s4)cc3c3cc(-c4nc5cccnc5s4)ccc3c2o1]

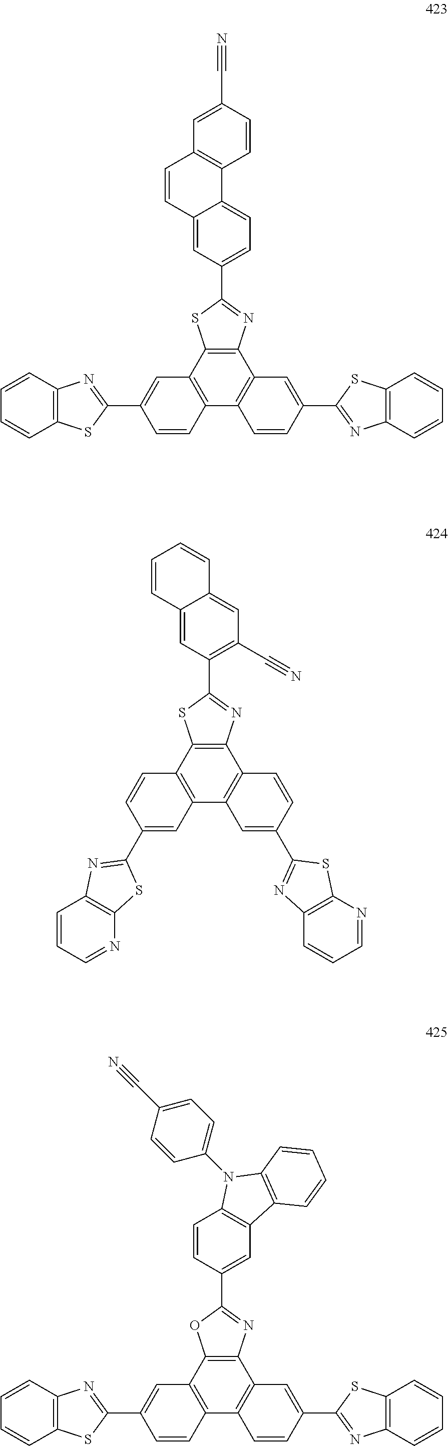 [SMILES: N#Cc1ccc2c(ccc3cc(-c4nc5c6cc(-c7nc8ccccc8s7)ccc6c6ccc(-c7nc8ccccc8s7)cc6c5s4)ccc32)c1 ; N#Cc1cc2ccccc2cc1-c1nc2c3ccc(-c4nc5cccnc5s4)cc3c3cc(-c4nc5cccnc5s4)ccc3c2s1 ; N#Cc1ccc(-n2c3ccccc3c3cc(-c4nc5c6cc(-c7nc8ccccc8s7)ccc6c6ccc(-c7nc8ccccc8s7)cc6c5o4)ccc32)cc1]

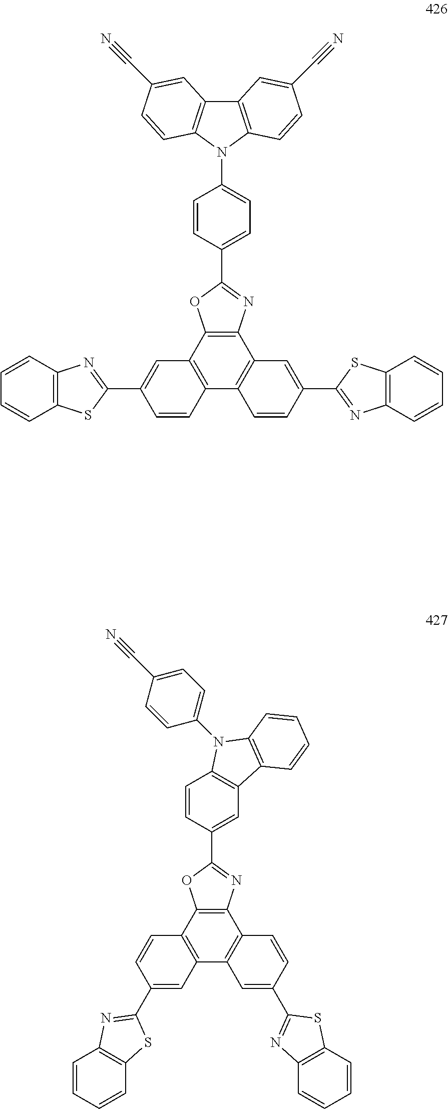 [SMILES: N#Cc1ccc2c(c1)c1cc(C#N)ccc1n2-c1ccc(-c2nc3c4cc(-c5nc6ccccc6s5)ccc4c4ccc(-c5nc6ccccc6s5)cc4c3o2)cc1 ; N#Cc1ccc(-n2c3ccccc3c3cc(-c4nc5c6ccc(-c7nc8ccccc8s7)cc6c6cc(-c7nc8ccccc8s7)ccc6c5o4)ccc32)cc1]

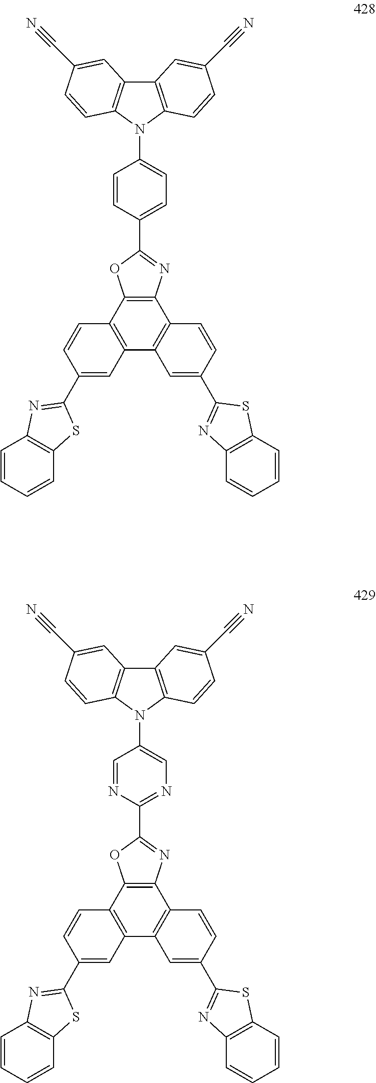 [SMILES: N#Cc1ccc2c(c1)c1cc(C#N)ccc1n2-c1ccc(-c2nc3c4ccc(-c5nc6ccccc6s5)cc4c4cc(-c5nc6ccccc6s5)ccc4c3o2)cc1 ; N#Cc1ccc2c(c1)c1cc(C#N)ccc1n2-c1cnc(-c2nc3c4ccc(-c5nc6ccccc6s5)cc4c4cc(-c5nc6ccccc6s5)ccc4c3o2)nc1]

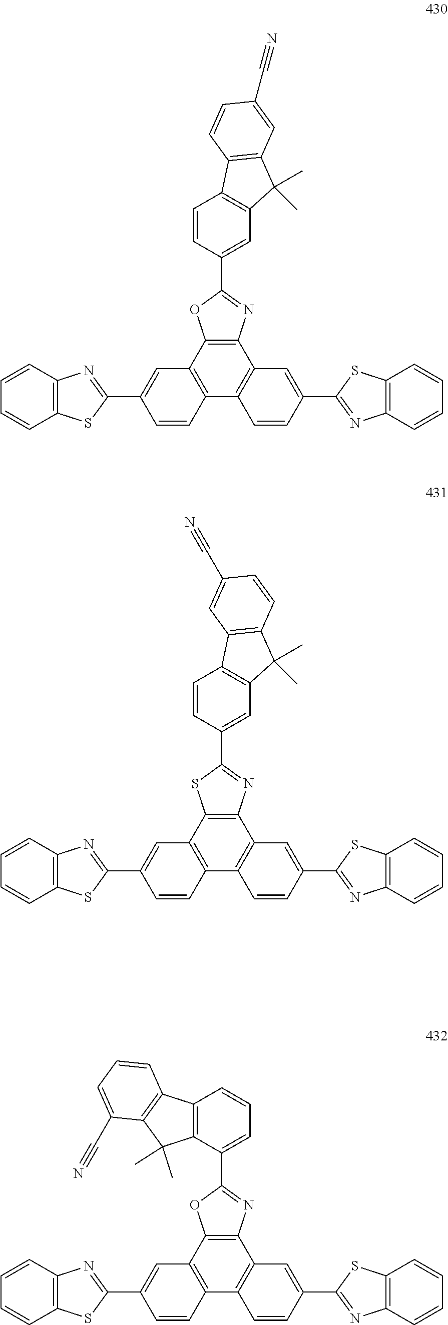 [SMILES: CC1(C)c2cc(C#N)ccc2-c2ccc(-c3nc4c5cc(-c6nc7ccccc7s6)ccc5c5ccc(-c6nc7ccccc7s6)cc5c4o3)cc21 ; CC1(C)c2ccc(C#N)cc2-c2ccc(-c3nc4c5cc(-c6nc7ccccc7s6)ccc5c5ccc(-c6nc7ccccc7s6)cc5c4s3)cc21 ; CC1(C)c2c(C#N)cccc2-c2cccc(-c3nc4c5cc(-c6nc7ccccc7s6)ccc5c5ccc(-c6nc7ccccc7s6)cc5c4o3)c21]

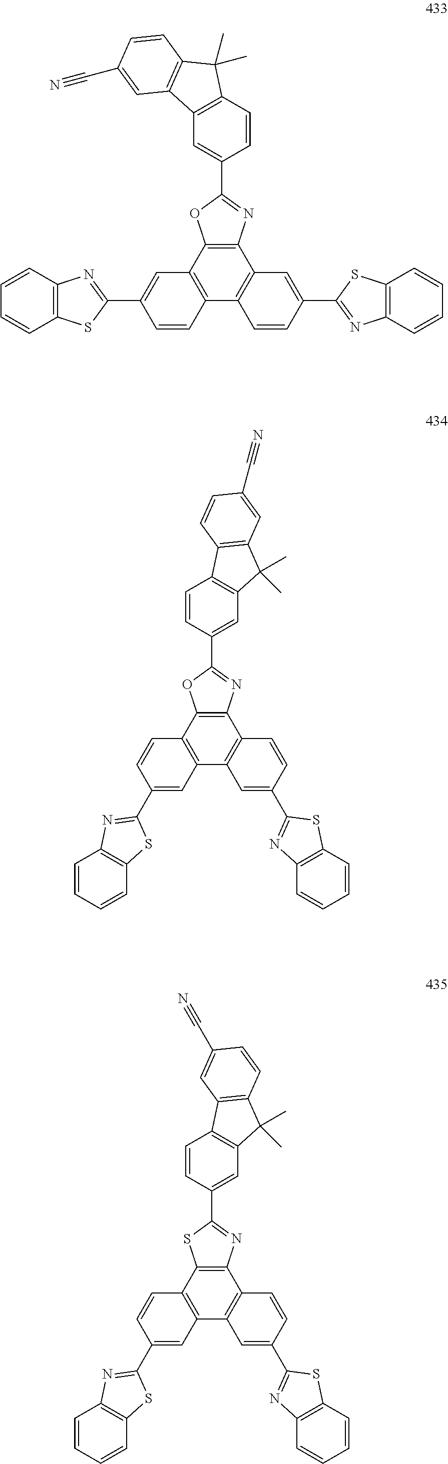 [SMILES: CC1(C)c2ccc(C#N)cc2-c2cc(-c3nc4c5cc(-c6nc7ccccc7s6)ccc5c5ccc(-c6nc7ccccc7s6)cc5c4o3)ccc21 ; CC1(C)c2cc(C#N)ccc2-c2ccc(-c3nc4c5ccc(-c6nc7ccccc7s6)cc5c5cc(-c6nc7ccccc7s6)ccc5c4o3)cc21 ; CC1(C)c2ccc(C#N)cc2-c2ccc(-c3nc4c5ccc(-c6nc7ccccc7s6)cc5c5cc(-c6nc7ccccc7s6)ccc5c4s3)cc21]

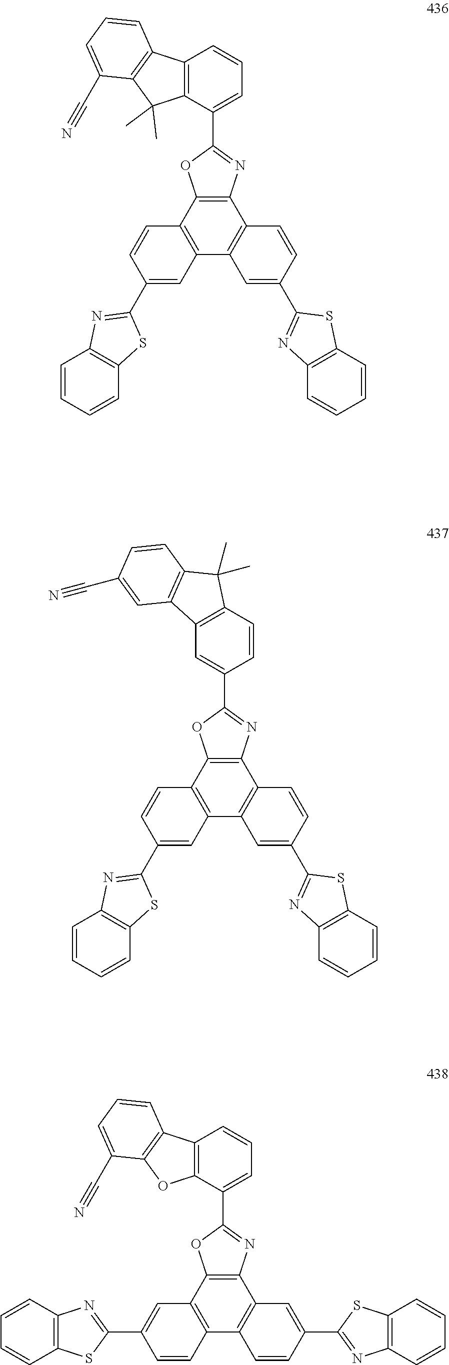 [SMILES: CC1(C)c2c(C#N)cccc2-c2cccc(-c3nc4c5ccc(-c6nc7ccccc7s6)cc5c5cc(-c6nc7ccccc7s6)ccc5c4o3)c21 ; CC1(C)c2ccc(C#N)cc2-c2cc(-c3nc4c5ccc(-c6nc7ccccc7s6)cc5c5cc(-c6nc7ccccc7s6)ccc5c4o3)ccc21 ; N#Cc1cccc2c1oc1c(-c3nc4c5cc(-c6nc7ccccc7s6)ccc5c5ccc(-c6nc7ccccc7s6)cc5c4o3)cccc12]

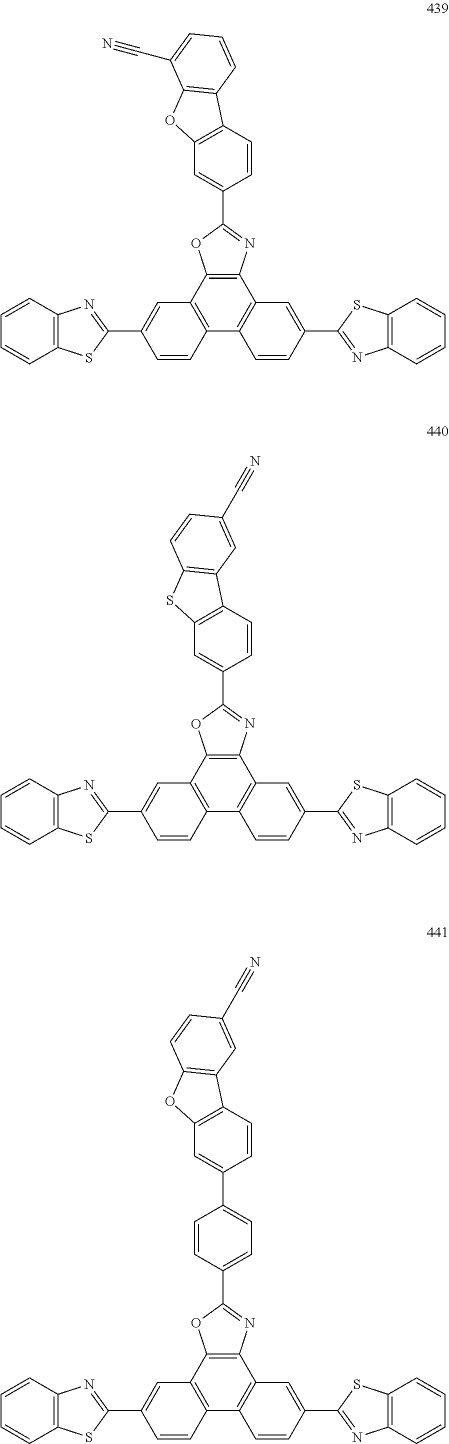 [SMILES: N#Cc1cccc2c1oc1cc(-c3nc4c5cc(-c6nc7ccccc7s6)ccc5c5ccc(-c6nc7ccccc7s6)cc5c4o3)ccc12 ; N#Cc1ccc2sc3cc(-c4nc5c6cc(-c7nc8ccccc8s7)ccc6c6ccc(-c7nc8ccccc8s7)cc6c5o4)ccc3c2c1 ; N#Cc1ccc2oc3cc(-c4ccc(-c5nc6c7cc(-c8nc9ccccc9s8)ccc7c7ccc(-c8nc9ccccc9s8)cc7c6o5)cc4)ccc3c2c1]

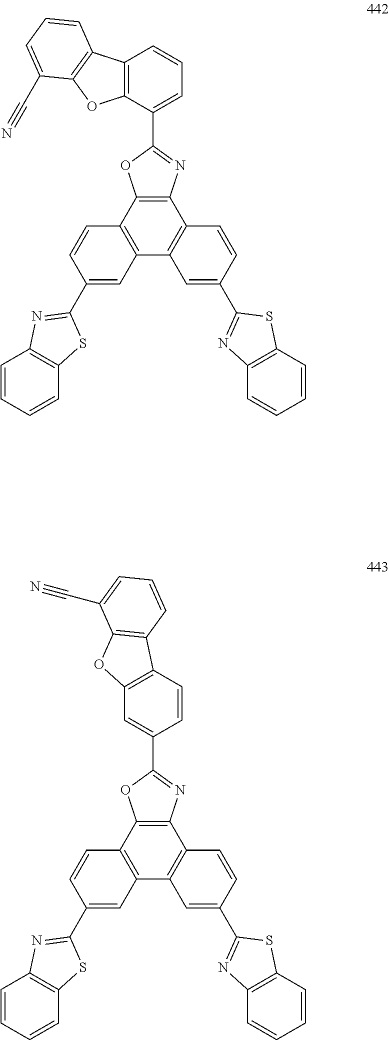 [SMILES: N#Cc1cccc2c1oc1c(-c3nc4c5ccc(-c6nc7ccccc7s6)cc5c5cc(-c6nc7ccccc7s6)ccc5c4o3)cccc12 ; N#Cc1cccc2c1oc1cc(-c3nc4c5ccc(-c6nc7ccccc7s6)cc5c5cc(-c6nc7ccccc7s6)ccc5c4o3)ccc12]

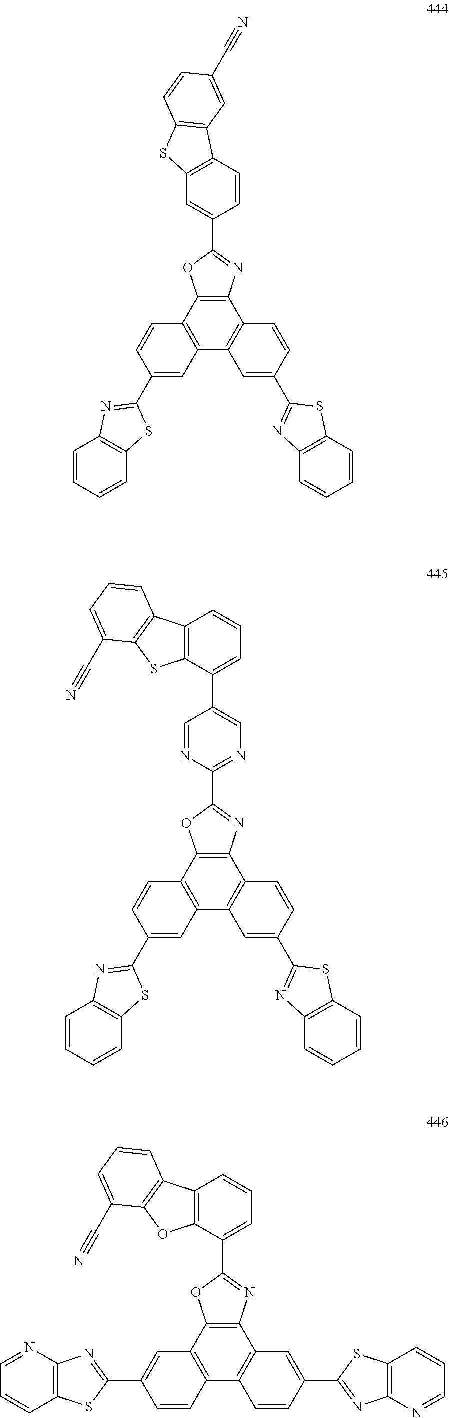 [SMILES: N#Cc1ccc2sc3cc(-c4nc5c6ccc(-c7nc8ccccc8s7)cc6c6cc(-c7nc8ccccc8s7)ccc6c5o4)ccc3c2c1 ; N#Cc1cccc2c1sc1c(-c3cnc(-c4nc5c6ccc(-c7nc8ccccc8s7)cc6c6cc(-c7nc8ccccc8s7)ccc6c5o4)nc3)cccc12 ; N#Cc1cccc2c1oc1c(-c3nc4c5cc(-c6nc7ncccc7s6)ccc5c5ccc(-c6nc7ncccc7s6)cc5c4o3)cccc12]

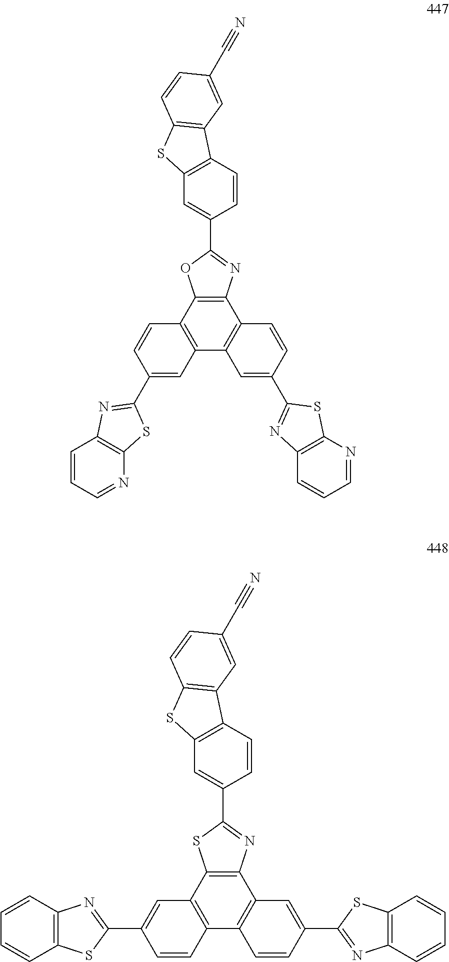 [SMILES: N#Cc1ccc2sc3cc(-c4nc5c6ccc(-c7nc8cccnc8s7)cc6c6cc(-c7nc8cccnc8s7)ccc6c5o4)ccc3c2c1 ; N#Cc1ccc2sc3cc(-c4nc5c6cc(-c7nc8ccccc8s7)ccc6c6ccc(-c7nc8ccccc8s7)cc6c5s4)ccc3c2c1]

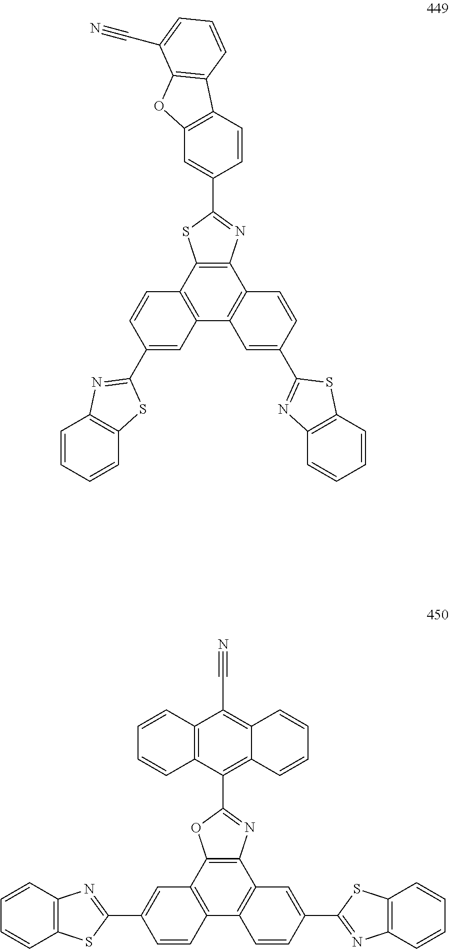 [SMILES: N#Cc1cccc2c1oc1cc(-c3nc4c5ccc(-c6nc7ccccc7s6)cc5c5cc(-c6nc7ccccc7s6)ccc5c4s3)ccc12 ; N#Cc1c2ccccc2c(-c2nc3c4cc(-c5nc6ccccc6s5)ccc4c4ccc(-c5nc6ccccc6s5)cc4c3o2)c2ccccc12]

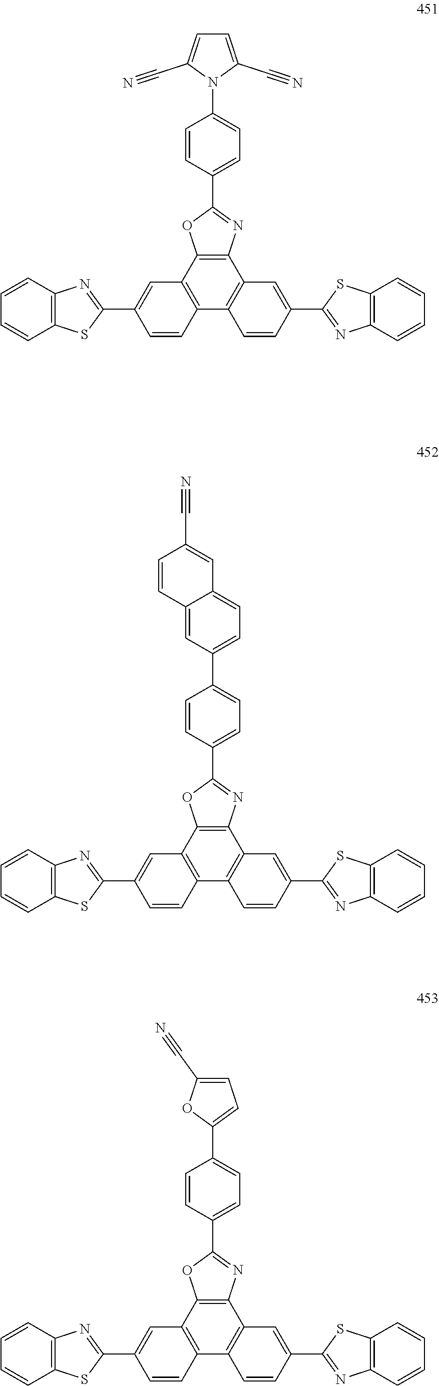 [SMILES: N#Cc1ccc(C#N)n1-c1ccc(-c2nc3c4cc(-c5nc6ccccc6s5)ccc4c4ccc(-c5nc6ccccc6s5)cc4c3o2)cc1 ; N#Cc1ccc2cc(-c3ccc(-c4nc5c6cc(-c7nc8ccccc8s7)ccc6c6ccc(-c7nc8ccccc8s7)cc6c5o4)cc3)ccc2c1 ; N#Cc1ccc(-c2ccc(-c3nc4c5cc(-c6nc7ccccc7s6)ccc5c5ccc(-c6nc7ccccc7s6)cc5c4o3)cc2)o1]

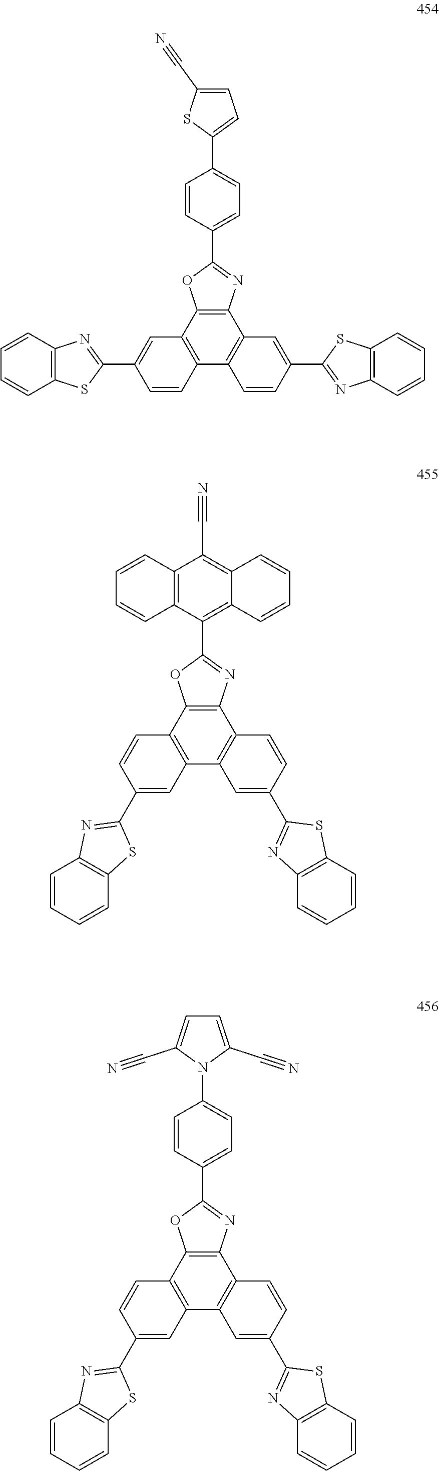 [SMILES: N#Cc1ccc(-c2ccc(-c3nc4c5cc(-c6nc7ccccc7s6)ccc5c5ccc(-c6nc7ccccc7s6)cc5c4o3)cc2)s1 ; N#Cc1c2ccccc2c(-c2nc3c4ccc(-c5nc6ccccc6s5)cc4c4cc(-c5nc6ccccc6s5)ccc4c3o2)c2ccccc12 ; N#Cc1ccc(C#N)n1-c1ccc(-c2nc3c4ccc(-c5nc6ccccc6s5)cc4c4cc(-c5nc6ccccc6s5)ccc4c3o2)cc1]

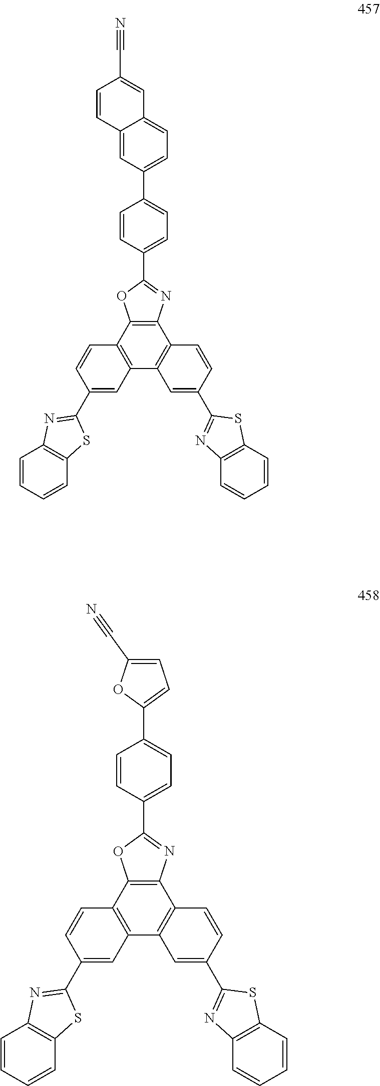 [SMILES: N#Cc1ccc2cc(-c3ccc(-c4nc5c6ccc(-c7nc8ccccc8s7)cc6c6cc(-c7nc8ccccc8s7)ccc6c5o4)cc3)ccc2c1 ; N#Cc1ccc(-c2ccc(-c3nc4c5ccc(-c6nc7ccccc7s6)cc5c5cc(-c6nc7ccccc7s6)ccc5c4o3)cc2)o1]

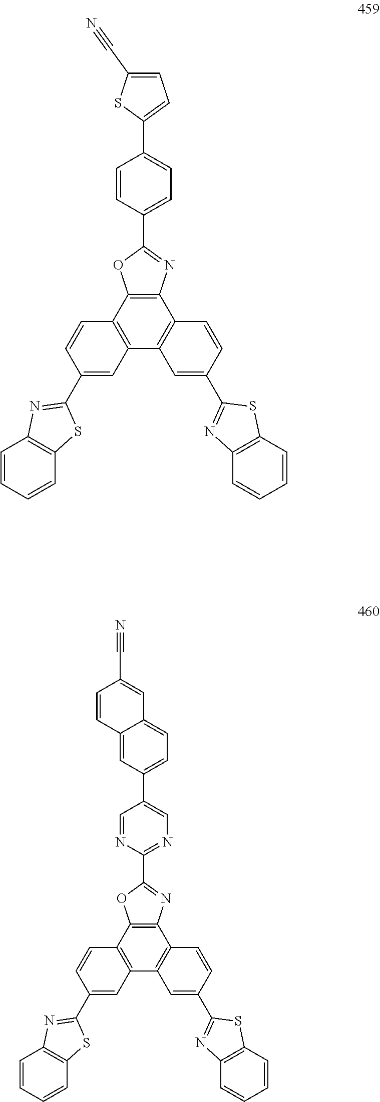 [SMILES: N#Cc1ccc(-c2ccc(-c3nc4c5ccc(-c6nc7ccccc7s6)cc5c5cc(-c6nc7ccccc7s6)ccc5c4o3)cc2)s1 ; N#Cc1ccc2cc(-c3cnc(-c4nc5c6ccc(-c7nc8ccccc8s7)cc6c6cc(-c7nc8ccccc8s7)ccc6c5o4)nc3)ccc2c1]

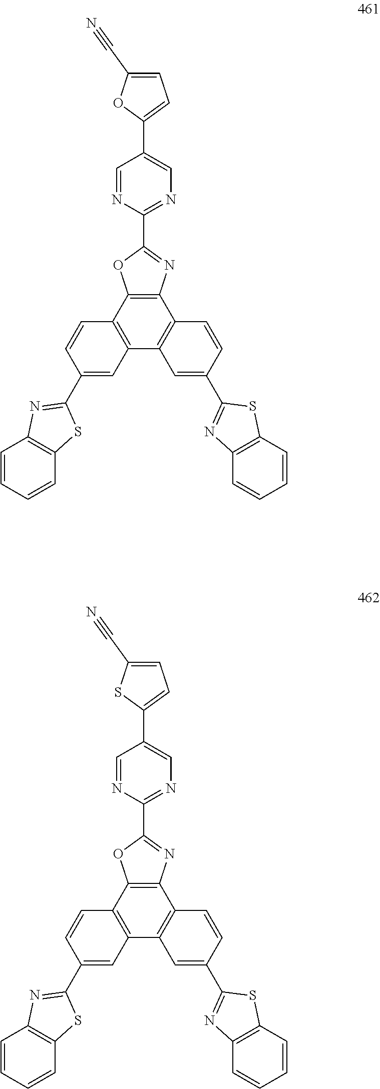 [SMILES: N#Cc1ccc(-c2cnc(-c3nc4c5ccc(-c6nc7ccccc7s6)cc5c5cc(-c6nc7ccccc7s6)ccc5c4o3)nc2)o1 ; N#Cc1ccc(-c2cnc(-c3nc4c5ccc(-c6nc7ccccc7s6)cc5c5cc(-c6nc7ccccc7s6)ccc5c4o3)nc2)s1]

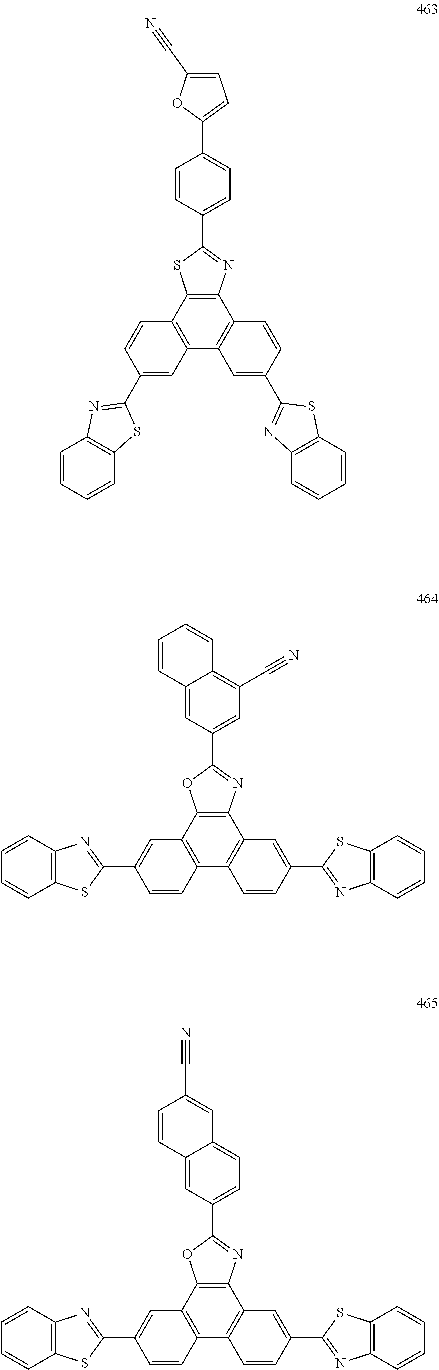 [SMILES: N#Cc1ccc(-c2ccc(-c3nc4c5ccc(-c6nc7ccccc7s6)cc5c5cc(-c6nc7ccccc7s6)ccc5c4s3)cc2)o1 ; N#Cc1cc(-c2nc3c4cc(-c5nc6ccccc6s5)ccc4c4ccc(-c5nc6ccccc6s5)cc4c3o2)cc2ccccc12 ; N#Cc1ccc2cc(-c3nc4c5cc(-c6nc7ccccc7s6)ccc5c5ccc(-c6nc7ccccc7s6)cc5c4o3)ccc2c1]

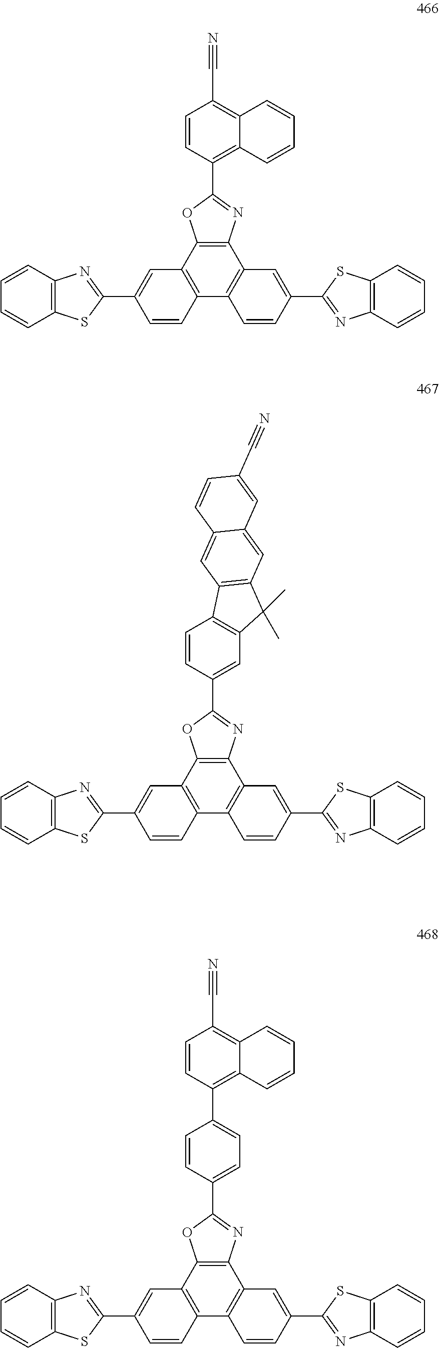 [SMILES: N#Cc1ccc(-c2nc3c4cc(-c5nc6ccccc6s5)ccc4c4ccc(-c5nc6ccccc6s5)cc4c3o2)c2ccccc12 ; CC1(C)c2cc(-c3nc4c5cc(-c6nc7ccccc7s6)ccc5c5ccc(-c6nc7ccccc7s6)cc5c4o3)ccc2-c2cc3ccc(C#N)cc3cc21 ; N#Cc1ccc(-c2ccc(-c3nc4c5cc(-c6nc7ccccc7s6)ccc5c5ccc(-c6nc7ccccc7s6)cc5c4o3)cc2)c2ccccc12]

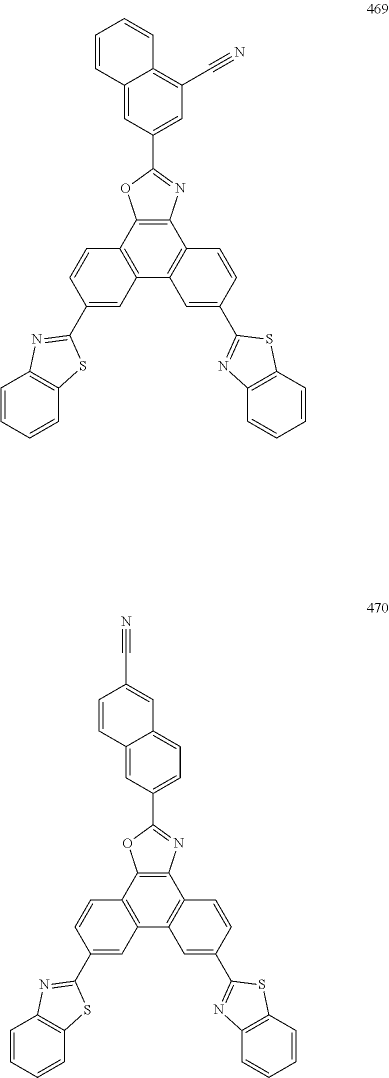 [SMILES: N#Cc1cc(-c2nc3c4ccc(-c5nc6ccccc6s5)cc4c4cc(-c5nc6ccccc6s5)ccc4c3o2)cc2ccccc12 ; N#Cc1ccc2cc(-c3nc4c5ccc(-c6nc7ccccc7s6)cc5c5cc(-c6nc7ccccc7s6)ccc5c4o3)ccc2c1]

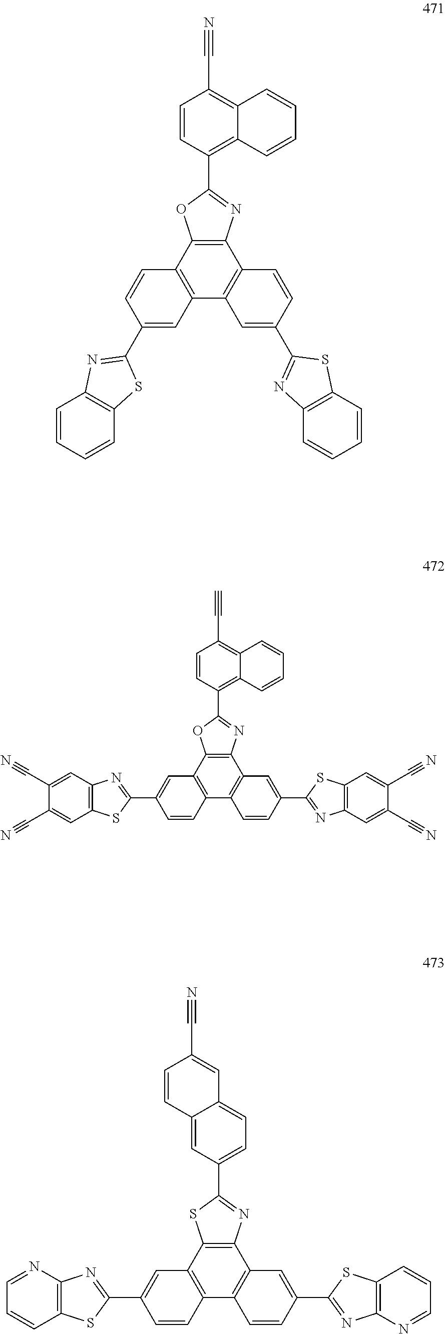 [SMILES: N#Cc1ccc(-c2nc3c4ccc(-c5nc6ccccc6s5)cc4c4cc(-c5nc6ccccc6s5)ccc4c3o2)c2ccccc12 ; C#Cc1ccc(-c2nc3c4cc(-c5nc6cc(C#N)c(C#N)cc6s5)ccc4c4ccc(-c5nc6cc(C#N)c(C#N)cc6s5)cc4c3o2)c2ccccc12 ; N#Cc1ccc2cc(-c3nc4c5cc(-c6nc7ncccc7s6)ccc5c5ccc(-c6nc7ncccc7s6)cc5c4s3)ccc2c1]

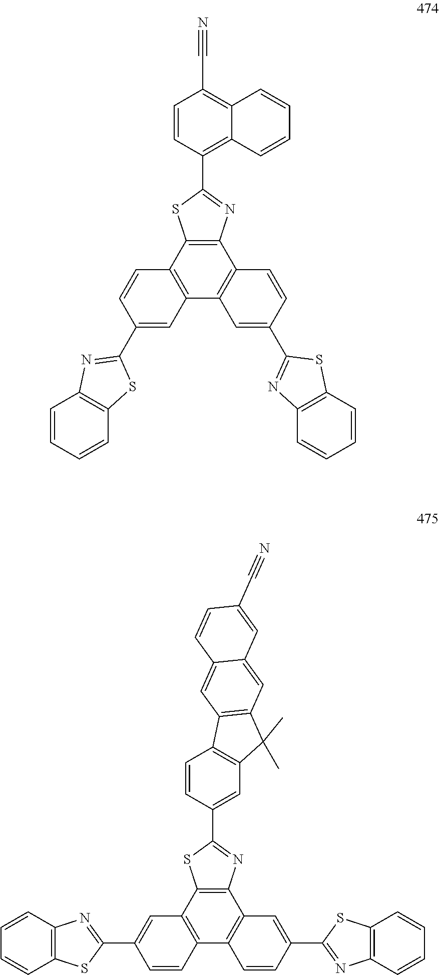 [SMILES: N#Cc1ccc(-c2nc3c4ccc(-c5nc6ccccc6s5)cc4c4cc(-c5nc6ccccc6s5)ccc4c3s2)c2ccccc12 ; CC1(C)c2cc(-c3nc4c5cc(-c6nc7ccccc7s6)ccc5c5ccc(-c6nc7ccccc7s6)cc5c4s3)ccc2-c2cc3ccc(C#N)cc3cc21]

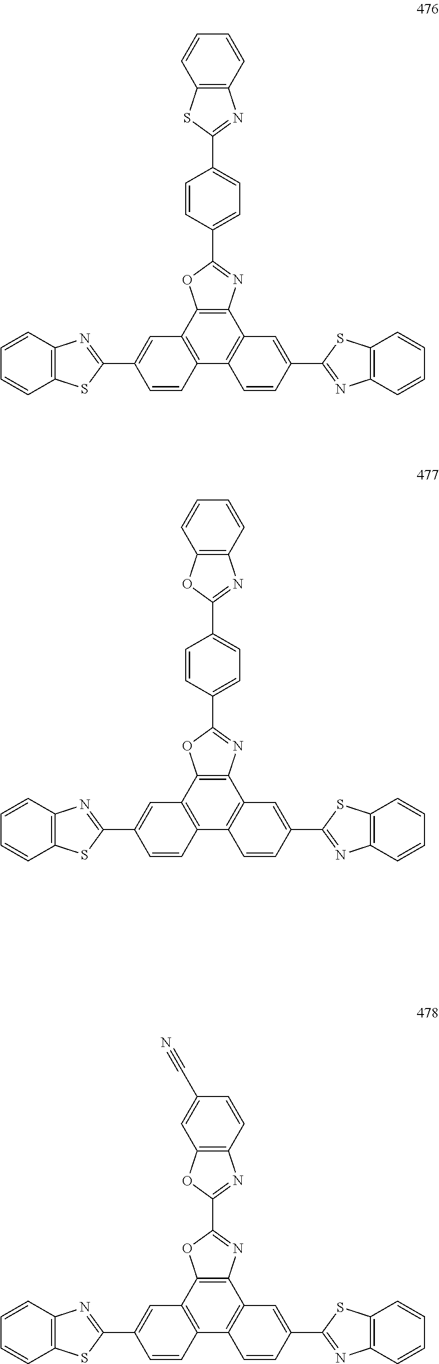 [SMILES: c1ccc2sc(-c3ccc(-c4nc5c6cc(-c7nc8ccccc8s7)ccc6c6ccc(-c7nc8ccccc8s7)cc6c5o4)cc3)nc2c1 ; c1ccc2oc(-c3ccc(-c4nc5c6cc(-c7nc8ccccc8s7)ccc6c6ccc(-c7nc8ccccc8s7)cc6c5o4)cc3)nc2c1 ; N#Cc1ccc2nc(-c3nc4c5cc(-c6nc7ccccc7s6)ccc5c5ccc(-c6nc7ccccc7s6)cc5c4o3)oc2c1]

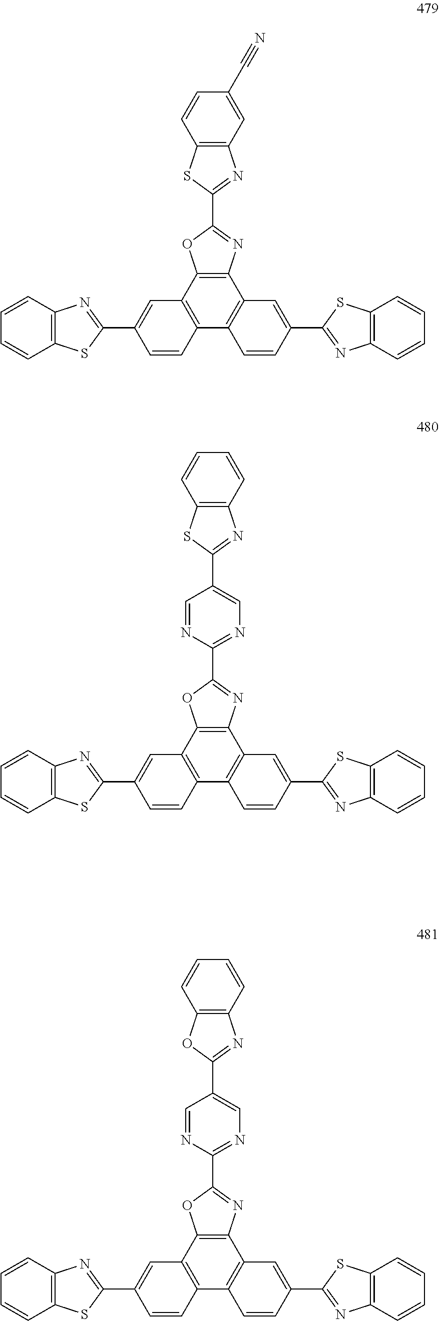 [SMILES: N#Cc1ccc2sc(-c3nc4c5cc(-c6nc7ccccc7s6)ccc5c5ccc(-c6nc7ccccc7s6)cc5c4o3)nc2c1 ; c1ccc2sc(-c3cnc(-c4nc5c6cc(-c7nc8ccccc8s7)ccc6c6ccc(-c7nc8ccccc8s7)cc6c5o4)nc3)nc2c1 ; c1ccc2oc(-c3cnc(-c4nc5c6cc(-c7nc8ccccc8s7)ccc6c6ccc(-c7nc8ccccc8s7)cc6c5o4)nc3)nc2c1]

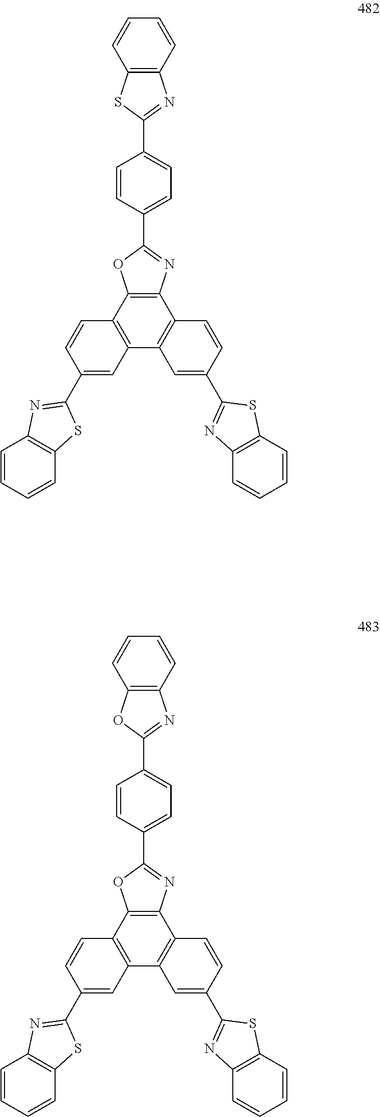 [SMILES: c1ccc2sc(-c3ccc(-c4nc5c6ccc(-c7nc8ccccc8s7)cc6c6cc(-c7nc8ccccc8s7)ccc6c5o4)cc3)nc2c1 ; c1ccc2oc(-c3ccc(-c4nc5c6ccc(-c7nc8ccccc8s7)cc6c6cc(-c7nc8ccccc8s7)ccc6c5o4)cc3)nc2c1]

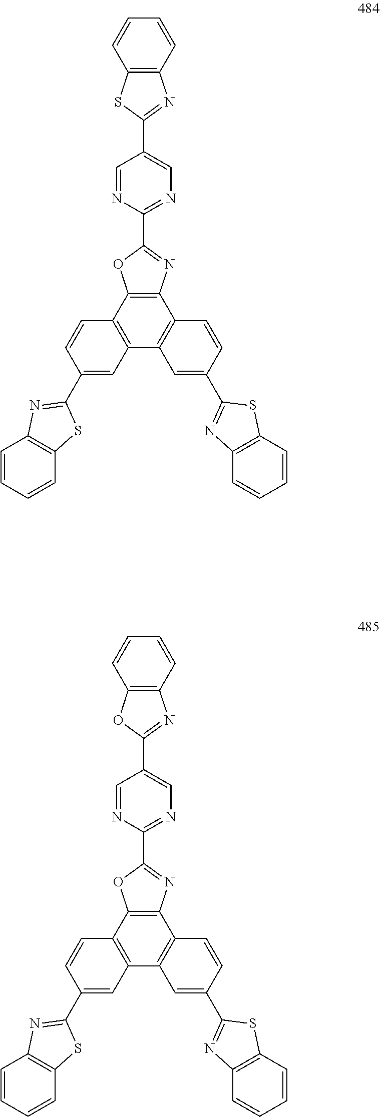 [SMILES: c1ccc2sc(-c3cnc(-c4nc5c6ccc(-c7nc8ccccc8s7)cc6c6cc(-c7nc8ccccc8s7)ccc6c5o4)nc3)nc2c1 ; c1ccc2oc(-c3cnc(-c4nc5c6ccc(-c7nc8ccccc8s7)cc6c6cc(-c7nc8ccccc8s7)ccc6c5o4)nc3)nc2c1]

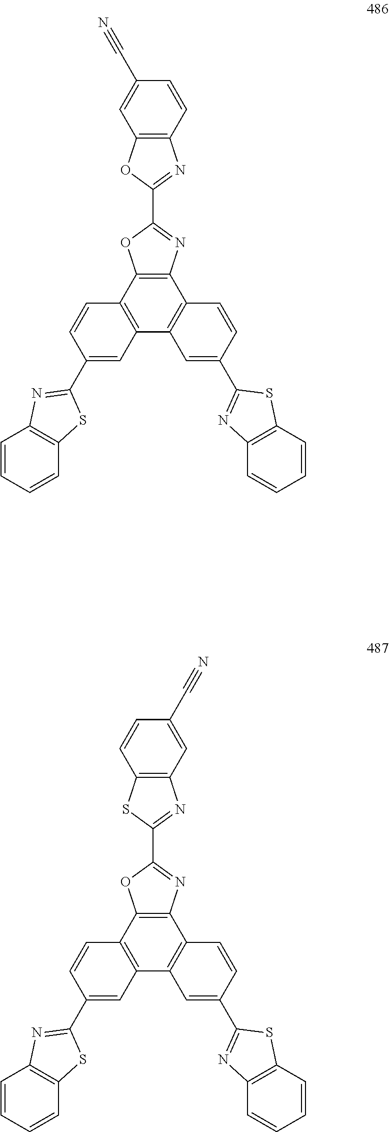 [SMILES: N#Cc1ccc2nc(-c3nc4c5ccc(-c6nc7ccccc7s6)cc5c5cc(-c6nc7ccccc7s6)ccc5c4o3)oc2c1 ; N#Cc1ccc2sc(-c3nc4c5ccc(-c6nc7ccccc7s6)cc5c5cc(-c6nc7ccccc7s6)ccc5c4o3)nc2c1]

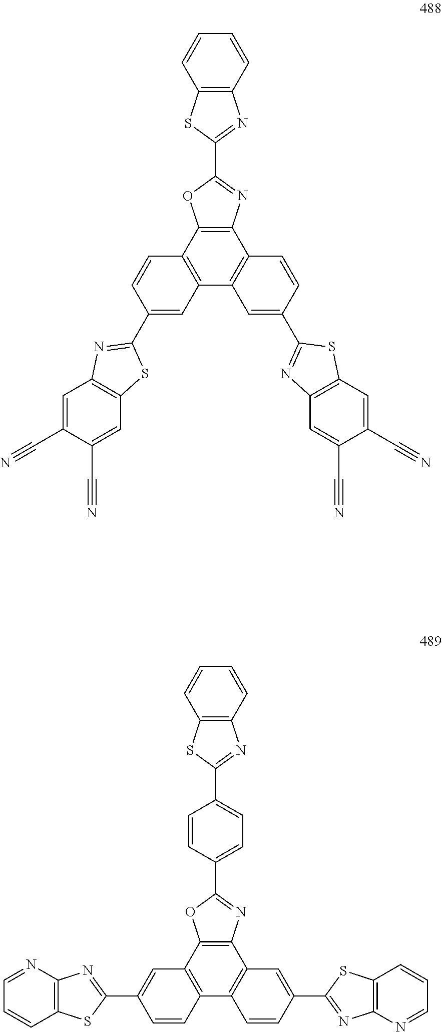 [SMILES: N#Cc1cc2nc(-c3ccc4c(c3)c3cc(-c5nc6cc(C#N)c(C#N)cc6s5)ccc3c3oc(-c5nc6ccccc6s5)nc43)sc2cc1C#N ; c1ccc2sc(-c3ccc(-c4nc5c6cc(-c7nc8ncccc8s7)ccc6c6ccc(-c7nc8ncccc8s7)cc6c5o4)cc3)nc2c1]

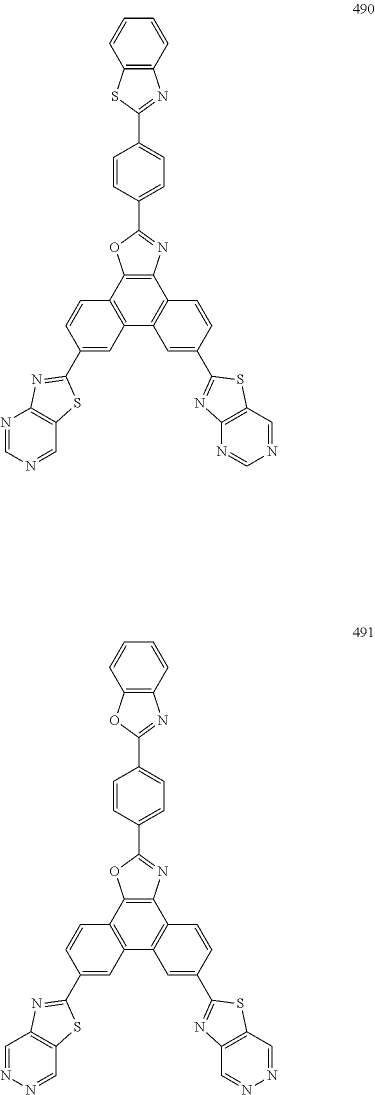 [SMILES: c1ccc2sc(-c3ccc(-c4nc5c6ccc(-c7nc8ncncc8s7)cc6c6cc(-c7nc8ncncc8s7)ccc6c5o4)cc3)nc2c1 ; c1ccc2oc(-c3ccc(-c4nc5c6ccc(-c7nc8cnncc8s7)cc6c6cc(-c7nc8cnncc8s7)ccc6c5o4)cc3)nc2c1]

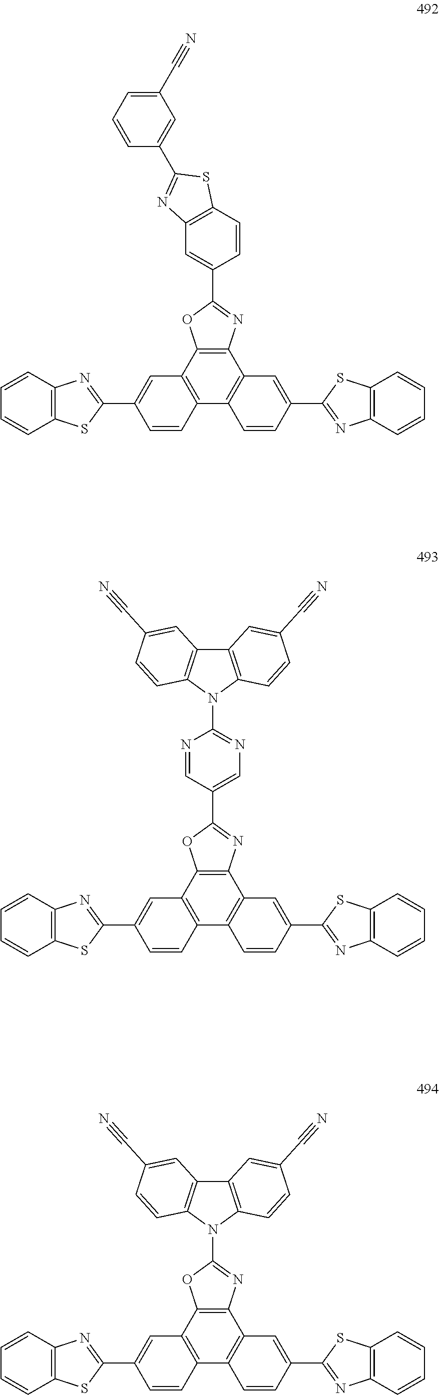 [SMILES: N#Cc1cccc(-c2nc3cc(-c4nc5c6cc(-c7nc8ccccc8s7)ccc6c6ccc(-c7nc8ccccc8s7)cc6c5o4)ccc3s2)c1 ; N#Cc1ccc2c(c1)c1cc(C#N)ccc1n2-c1ncc(-c2nc3c4cc(-c5nc6ccccc6s5)ccc4c4ccc(-c5nc6ccccc6s5)cc4c3o2)cn1 ; N#Cc1ccc2c(c1)c1cc(C#N)ccc1n2-c1nc2c3cc(-c4nc5ccccc5s4)ccc3c3ccc(-c4nc5ccccc5s4)cc3c2o1]

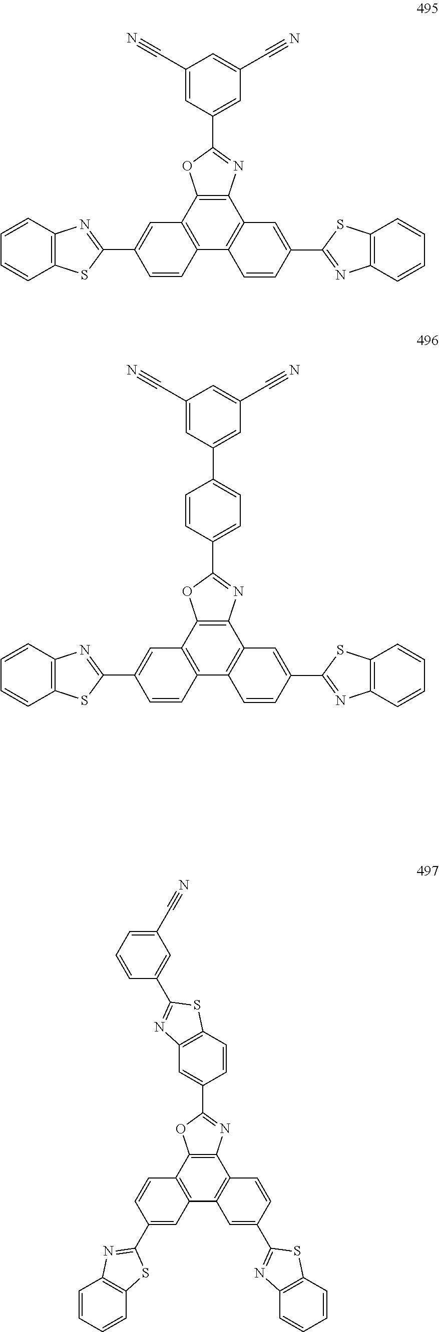 [SMILES: N#Cc1cc(C#N)cc(-c2nc3c4cc(-c5nc6ccccc6s5)ccc4c4ccc(-c5nc6ccccc6s5)cc4c3o2)c1 ; N#Cc1cc(C#N)cc(-c2ccc(-c3nc4c5cc(-c6nc7ccccc7s6)ccc5c5ccc(-c6nc7ccccc7s6)cc5c4o3)cc2)c1 ; N#Cc1cccc(-c2nc3cc(-c4nc5c6ccc(-c7nc8ccccc8s7)cc6c6cc(-c7nc8ccccc8s7)ccc6c5o4)ccc3s2)c1]

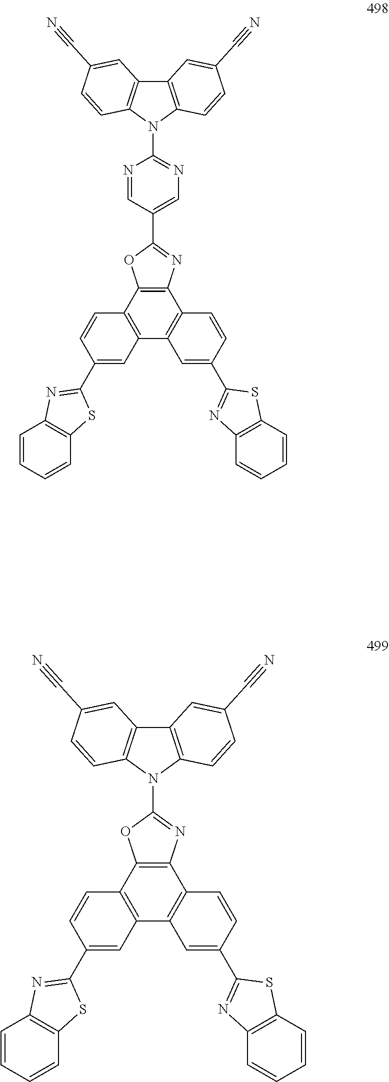 [SMILES: N#Cc1ccc2c(c1)c1cc(C#N)ccc1n2-c1ncc(-c2nc3c4ccc(-c5nc6ccccc6s5)cc4c4cc(-c5nc6ccccc6s5)ccc4c3o2)cn1 ; N#Cc1ccc2c(c1)c1cc(C#N)ccc1n2-c1nc2c3ccc(-c4nc5ccccc5s4)cc3c3cc(-c4nc5ccccc5s4)ccc3c2o1]

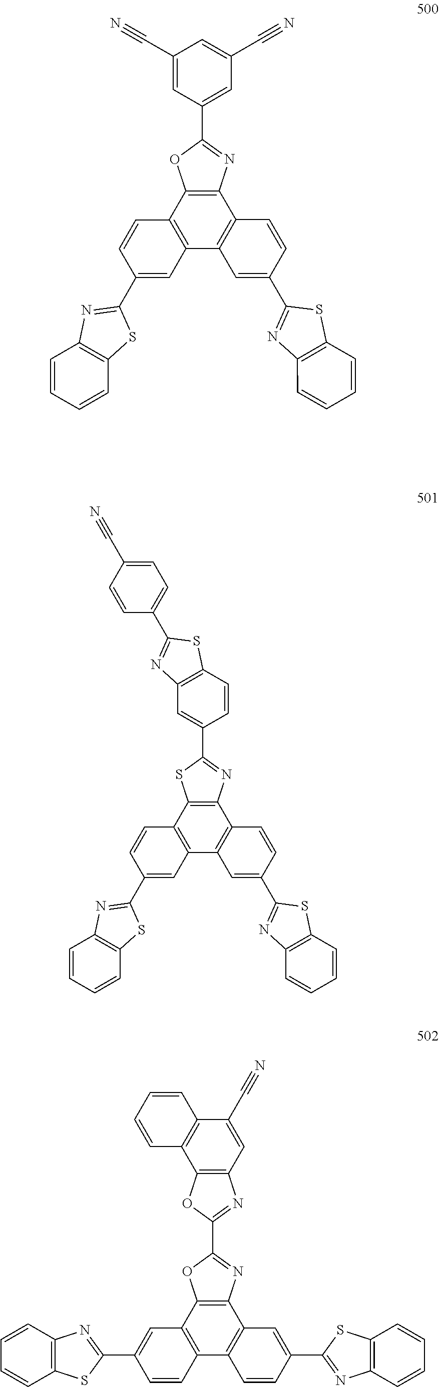 [SMILES: N#Cc1cc(C#N)cc(-c2nc3c4ccc(-c5nc6ccccc6s5)cc4c4cc(-c5nc6ccccc6s5)ccc4c3o2)c1 ; N#Cc1ccc(-c2nc3cc(-c4nc5c6ccc(-c7nc8ccccc8s7)cc6c6cc(-c7nc8ccccc8s7)ccc6c5s4)ccc3s2)cc1 ; N#Cc1cc2nc(-c3nc4c5cc(-c6nc7ccccc7s6)ccc5c5ccc(-c6nc7ccccc7s6)cc5c4o3)oc2c2ccccc12]

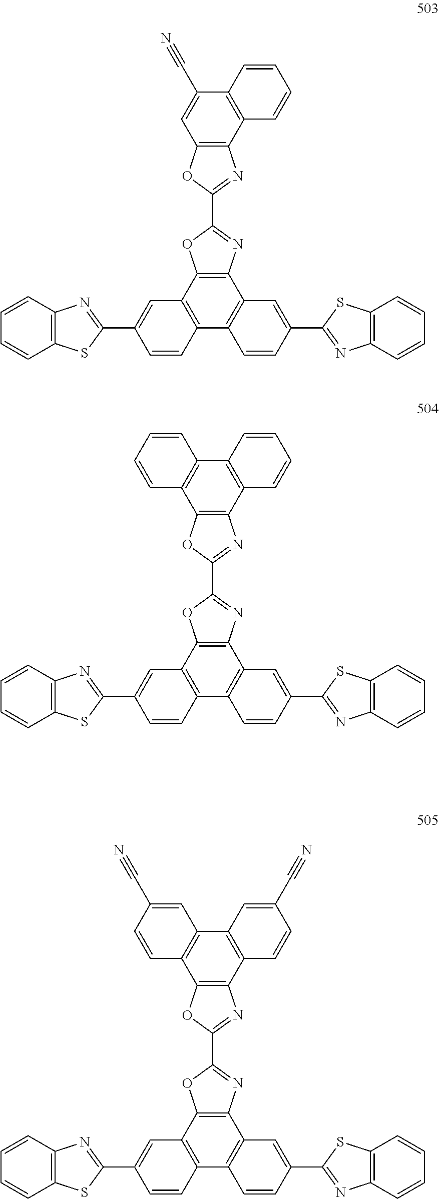 [SMILES: N#Cc1cc2oc(-c3nc4c5cc(-c6nc7ccccc7s6)ccc5c5ccc(-c6nc7ccccc7s6)cc5c4o3)nc2c2ccccc12 ; c1ccc2sc(-c3ccc4c5ccc(-c6nc7ccccc7s6)cc5c5oc(-c6nc7c8ccccc8c8ccccc8c7o6)nc5c4c3)nc2c1 ; N#Cc1ccc2c(c1)c1cc(C#N)ccc1c1oc(-c3nc4c5cc(-c6nc7ccccc7s6)ccc5c5ccc(-c6nc7ccccc7s6)cc5c4o3)nc21]

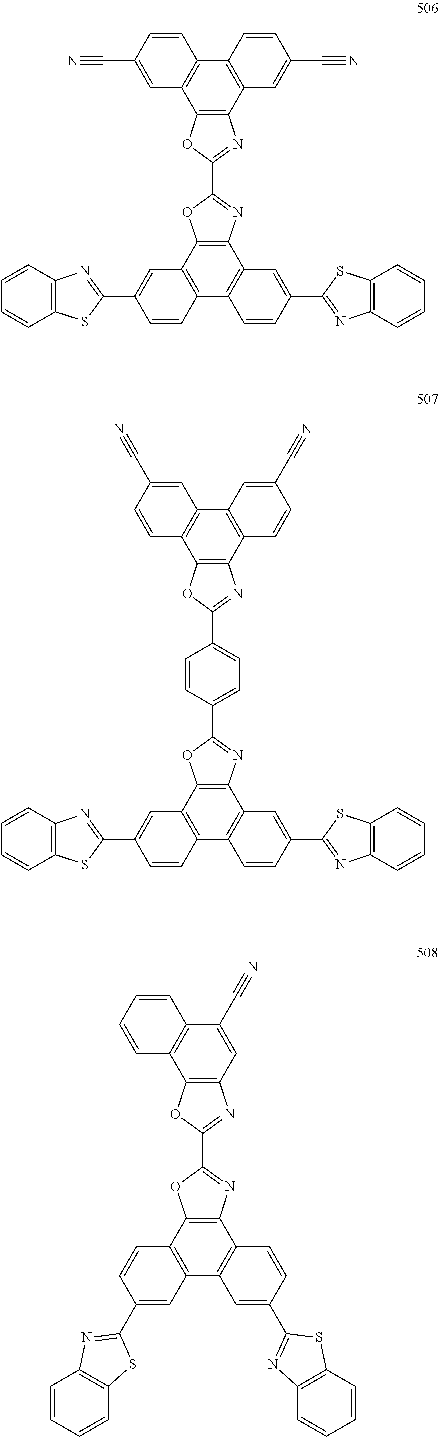 [SMILES: N#Cc1ccc2c3ccc(C#N)cc3c3oc(-c4nc5c6cc(-c7nc8ccccc8s7)ccc6c6ccc(-c7nc8ccccc8s7)cc6c5o4)nc3c2c1 ; N#Cc1ccc2c(c1)c1cc(C#N)ccc1c1oc(-c3ccc(-c4nc5c6cc(-c7nc8ccccc8s7)ccc6c6ccc(-c7nc8ccccc8s7)cc6c5o4)cc3)nc21 ; N#Cc1cc2nc(-c3nc4c5ccc(-c6nc7ccccc7s6)cc5c5cc(-c6nc7ccccc7s6)ccc5c4o3)oc2c2ccccc12]

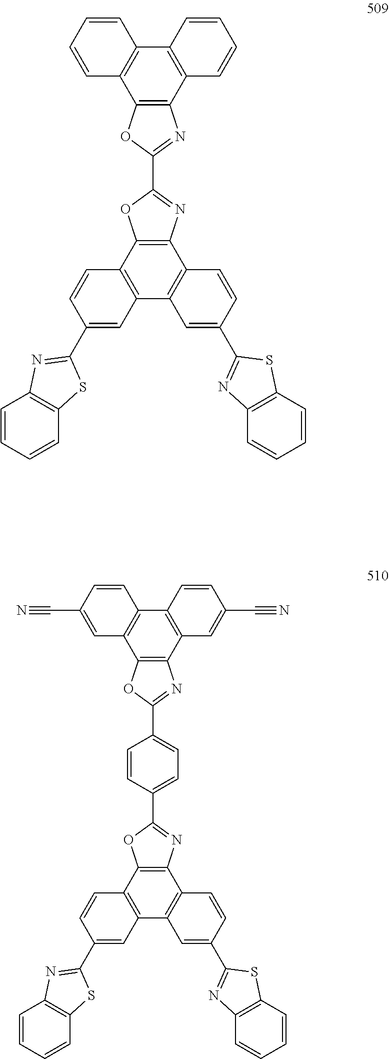 [SMILES: c1ccc2sc(-c3ccc4c(c3)c3cc(-c5nc6ccccc6s5)ccc3c3oc(-c5nc6c7ccccc7c7ccccc7c6o5)nc43)nc2c1 ; N#Cc1ccc2c3ccc(C#N)cc3c3oc(-c4ccc(-c5nc6c7ccc(-c8nc9ccccc9s8)cc7c7cc(-c8nc9ccccc9s8)ccc7c6o5)cc4)nc3c2c1]

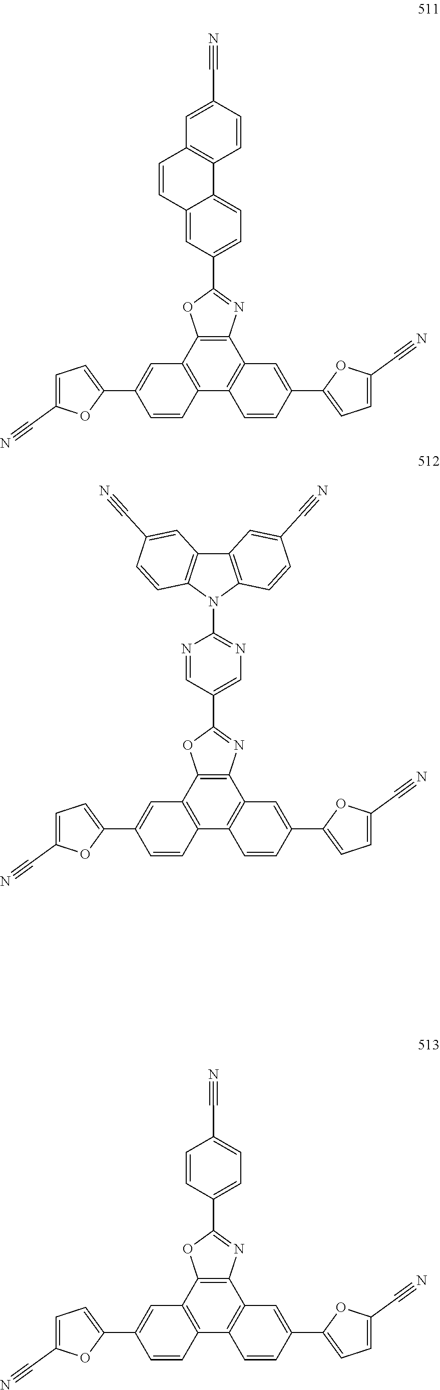 [SMILES: N#Cc1ccc2c(ccc3cc(-c4nc5c6cc(-c7ccc(C#N)o7)ccc6c6ccc(-c7ccc(C#N)o7)cc6c5o4)ccc32)c1 ; N#Cc1ccc2c(c1)c1cc(C#N)ccc1n2-c1ncc(-c2nc3c4cc(-c5ccc(C#N)o5)ccc4c4ccc(-c5ccc(C#N)o5)cc4c3o2)cn1 ; N#Cc1ccc(-c2nc3c4cc(-c5ccc(C#N)o5)ccc4c4ccc(-c5ccc(C#N)o5)cc4c3o2)cc1]

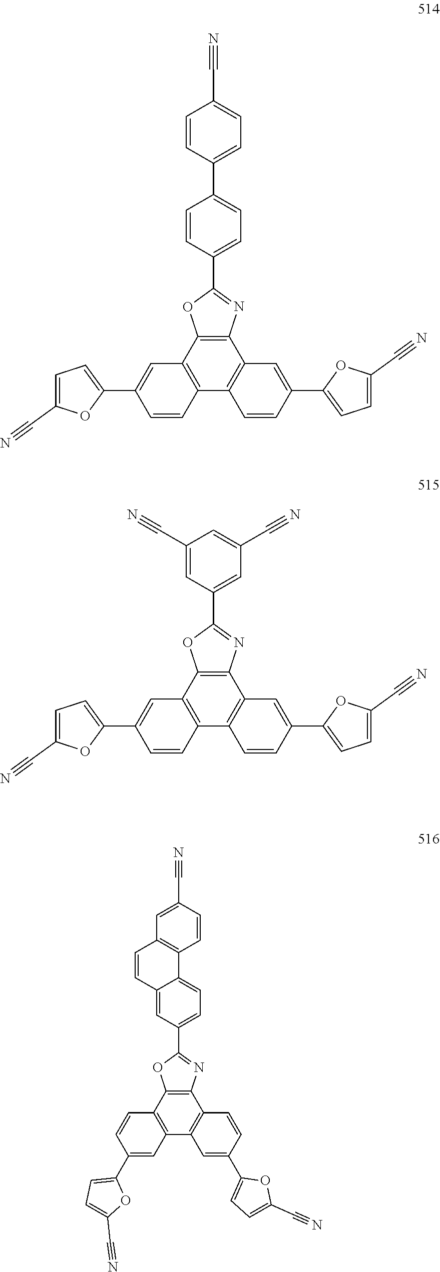 [SMILES: N#Cc1ccc(-c2ccc(-c3nc4c5cc(-c6ccc(C#N)o6)ccc5c5ccc(-c6ccc(C#N)o6)cc5c4o3)cc2)cc1 ; N#Cc1cc(C#N)cc(-c2nc3c4cc(-c5ccc(C#N)o5)ccc4c4ccc(-c5ccc(C#N)o5)cc4c3o2)c1 ; N#Cc1ccc2c(ccc3cc(-c4nc5c6ccc(-c7ccc(C#N)o7)cc6c6cc(-c7ccc(C#N)o7)ccc6c5o4)ccc32)c1]

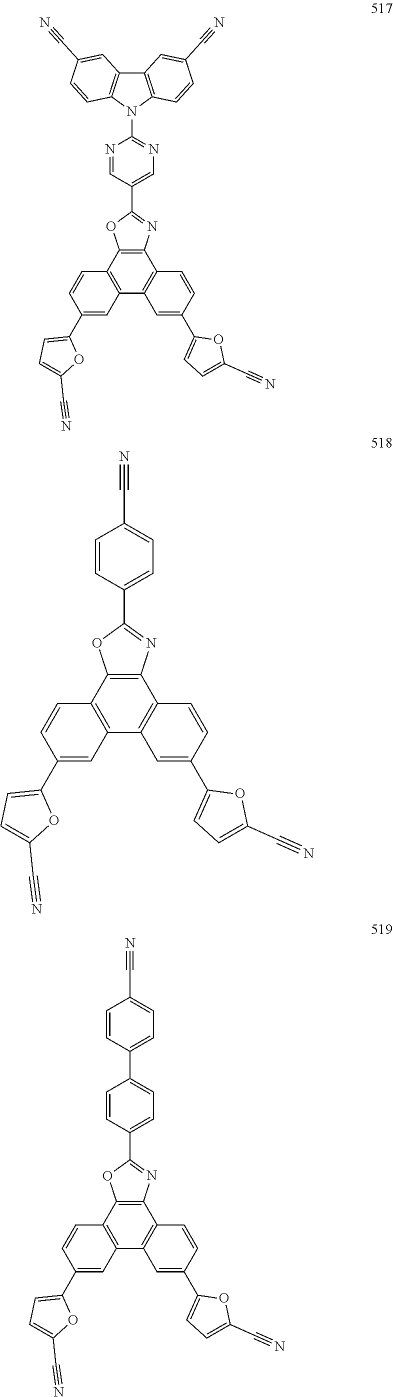 [SMILES: N#Cc1ccc2c(c1)c1cc(C#N)ccc1n2-c1ncc(-c2nc3c4ccc(-c5ccc(C#N)o5)cc4c4cc(-c5ccc(C#N)o5)ccc4c3o2)cn1 ; N#Cc1ccc(-c2nc3c4ccc(-c5ccc(C#N)o5)cc4c4cc(-c5ccc(C#N)o5)ccc4c3o2)cc1 ; N#Cc1ccc(-c2ccc(-c3nc4c5ccc(-c6ccc(C#N)o6)cc5c5cc(-c6ccc(C#N)o6)ccc5c4o3)cc2)cc1]

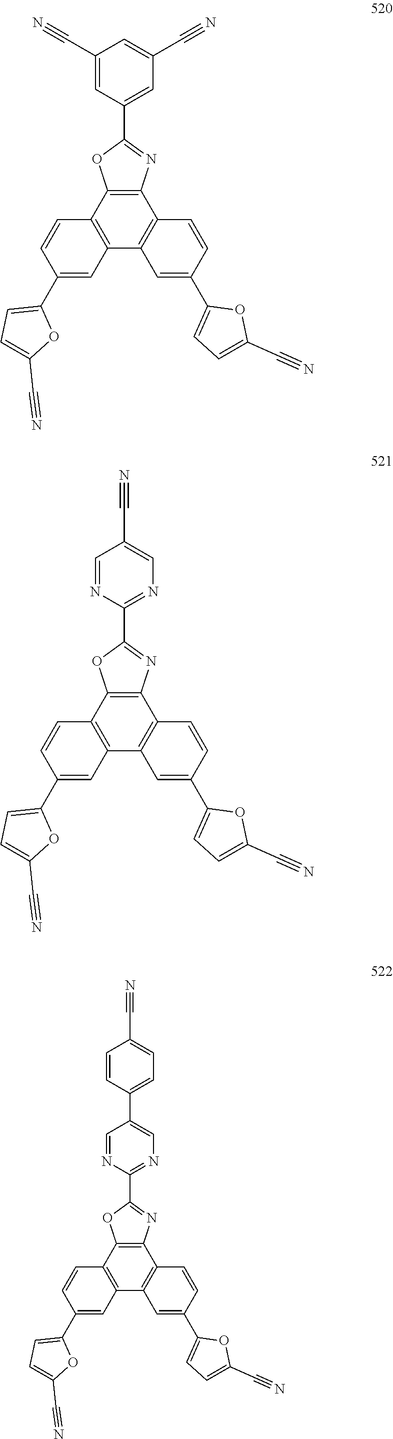 [SMILES: N#Cc1cc(C#N)cc(-c2nc3c4ccc(-c5ccc(C#N)o5)cc4c4cc(-c5ccc(C#N)o5)ccc4c3o2)c1 ; N#Cc1cnc(-c2nc3c4ccc(-c5ccc(C#N)o5)cc4c4cc(-c5ccc(C#N)o5)ccc4c3o2)nc1 ; N#Cc1ccc(-c2cnc(-c3nc4c5ccc(-c6ccc(C#N)o6)cc5c5cc(-c6ccc(C#N)o6)ccc5c4o3)nc2)cc1]

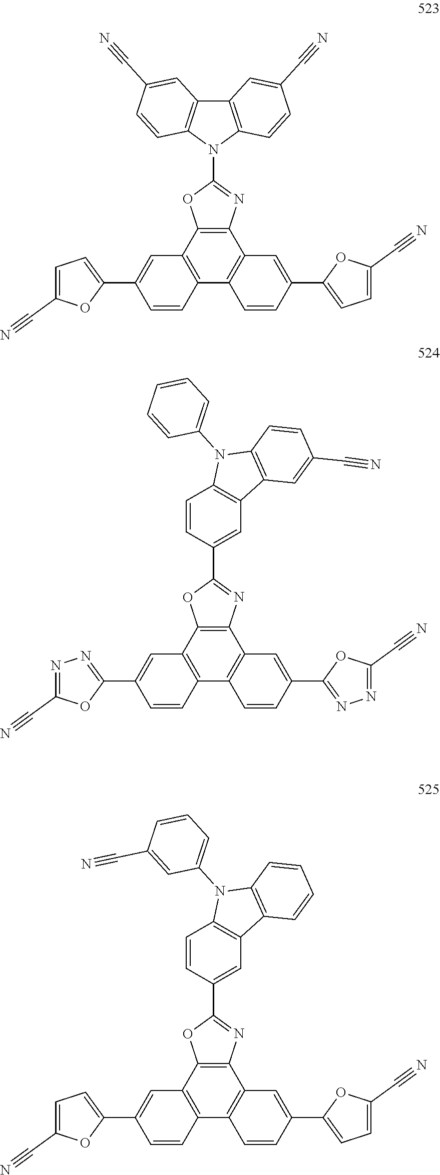 [SMILES: N#Cc1ccc2c(c1)c1cc(C#N)ccc1n2-c1nc2c3cc(-c4ccc(C#N)o4)ccc3c3ccc(-c4ccc(C#N)o4)cc3c2o1 ; N#Cc1ccc2c(c1)c1cc(-c3nc4c5cc(-c6nnc(C#N)o6)ccc5c5ccc(-c6nnc(C#N)o6)cc5c4o3)ccc1n2-c1ccccc1 ; N#Cc1cccc(-n2c3ccccc3c3cc(-c4nc5c6cc(-c7ccc(C#N)o7)ccc6c6ccc(-c7ccc(C#N)o7)cc6c5o4)ccc32)c1]

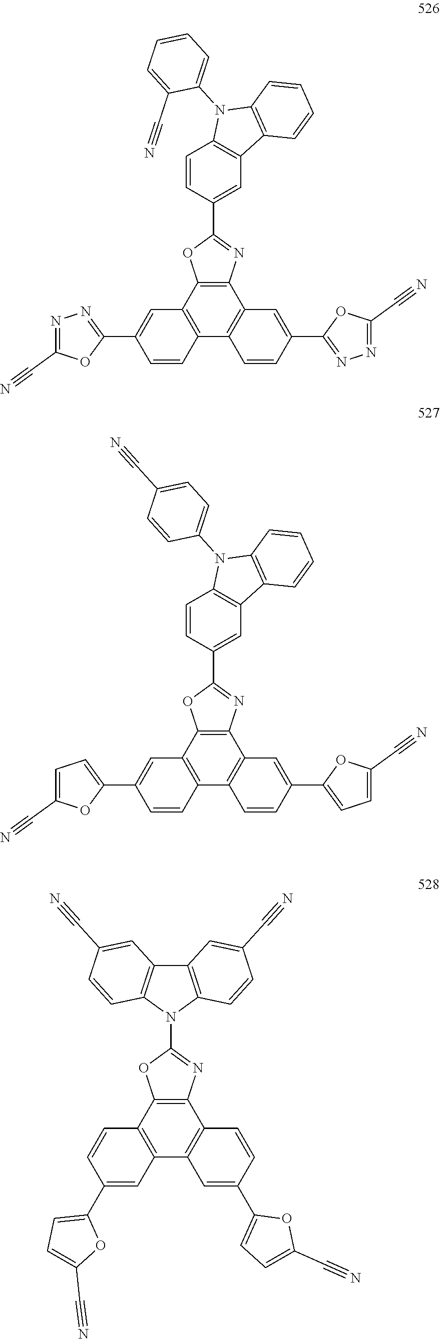 [SMILES: N#Cc1nnc(-c2ccc3c4ccc(-c5nnc(C#N)o5)cc4c4oc(-c5ccc6c(c5)c5ccccc5n6-c5ccccc5C#N)nc4c3c2)o1 ; N#Cc1ccc(-n2c3ccccc3c3cc(-c4nc5c6cc(-c7ccc(C#N)o7)ccc6c6ccc(-c7ccc(C#N)o7)cc6c5o4)ccc32)cc1 ; N#Cc1ccc2c(c1)c1cc(C#N)ccc1n2-c1nc2c3ccc(-c4ccc(C#N)o4)cc3c3cc(-c4ccc(C#N)o4)ccc3c2o1]

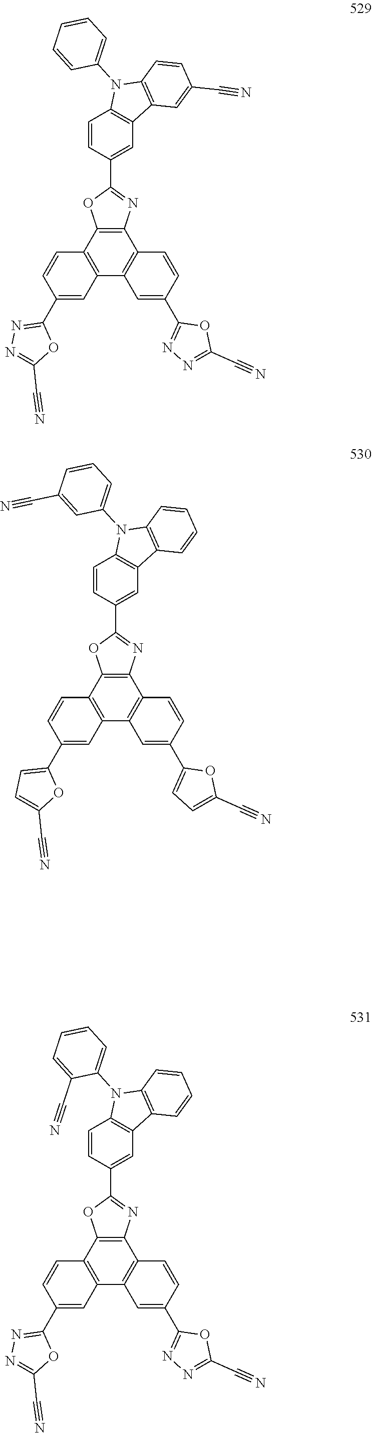 [SMILES: N#Cc1ccc2c(c1)c1cc(-c3nc4c5ccc(-c6nnc(C#N)o6)cc5c5cc(-c6nnc(C#N)o6)ccc5c4o3)ccc1n2-c1ccccc1 ; N#Cc1cccc(-n2c3ccccc3c3cc(-c4nc5c6ccc(-c7ccc(C#N)o7)cc6c6cc(-c7ccc(C#N)o7)ccc6c5o4)ccc32)c1 ; N#Cc1nnc(-c2ccc3c(c2)c2cc(-c4nnc(C#N)o4)ccc2c2oc(-c4ccc5c(c4)c4ccccc4n5-c4ccccc4C#N)nc32)o1]

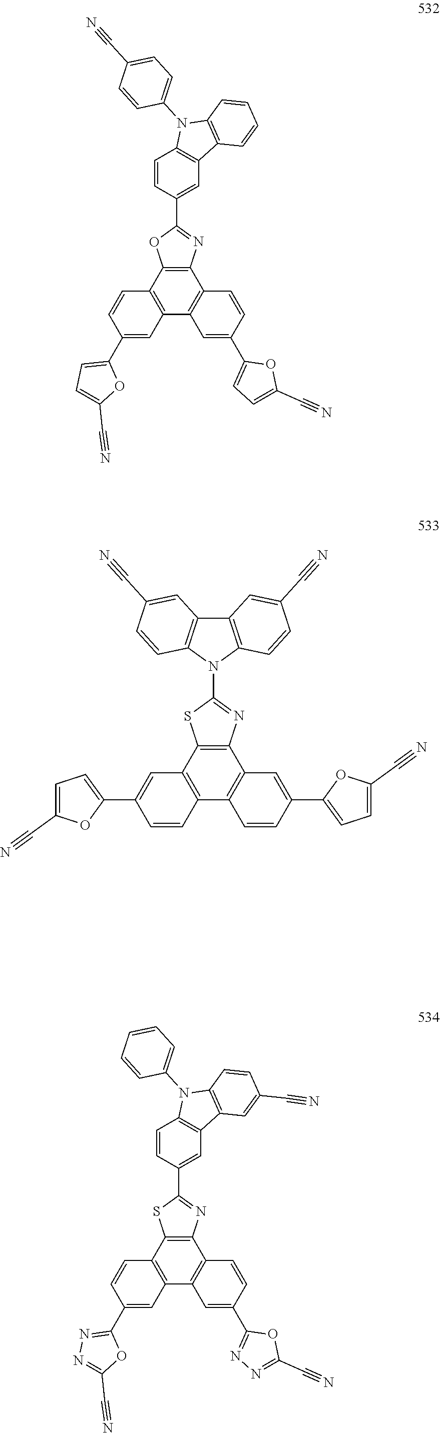 [SMILES: N#Cc1ccc(-n2c3ccccc3c3cc(-c4nc5c6ccc(-c7ccc(C#N)o7)cc6c6cc(-c7ccc(C#N)o7)ccc6c5o4)ccc32)cc1 ; N#Cc1ccc2c(c1)c1cc(C#N)ccc1n2-c1nc2c3cc(-c4ccc(C#N)o4)ccc3c3ccc(-c4ccc(C#N)o4)cc3c2s1 ; N#Cc1ccc2c(c1)c1cc(-c3nc4c5ccc(-c6nnc(C#N)o6)cc5c5cc(-c6nnc(C#N)o6)ccc5c4s3)ccc1n2-c1ccccc1]

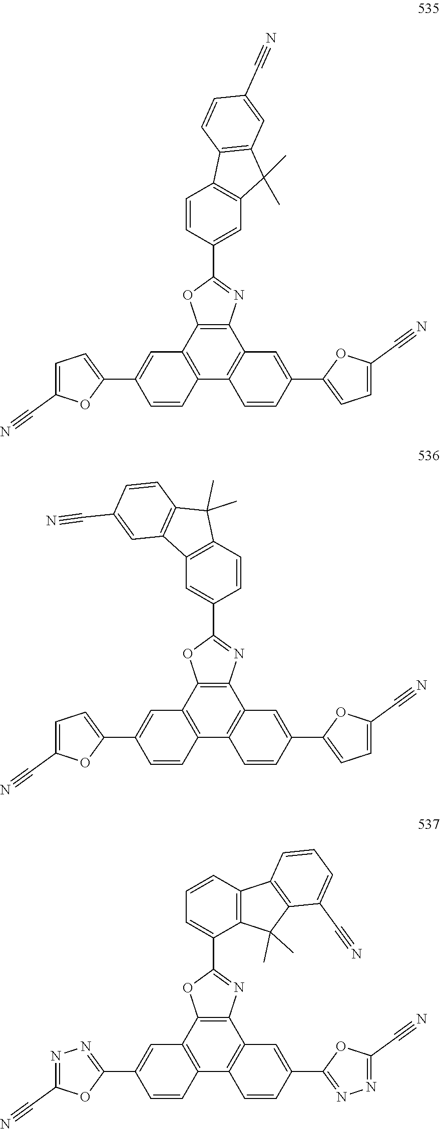 [SMILES: CC1(C)c2cc(C#N)ccc2-c2ccc(-c3nc4c5cc(-c6ccc(C#N)o6)ccc5c5ccc(-c6ccc(C#N)o6)cc5c4o3)cc21 ; CC1(C)c2ccc(C#N)cc2-c2cc(-c3nc4c5cc(-c6ccc(C#N)o6)ccc5c5ccc(-c6ccc(C#N)o6)cc5c4o3)ccc21 ; CC1(C)c2c(C#N)cccc2-c2cccc(-c3nc4c5cc(-c6nnc(C#N)o6)ccc5c5ccc(-c6nnc(C#N)o6)cc5c4o3)c21]

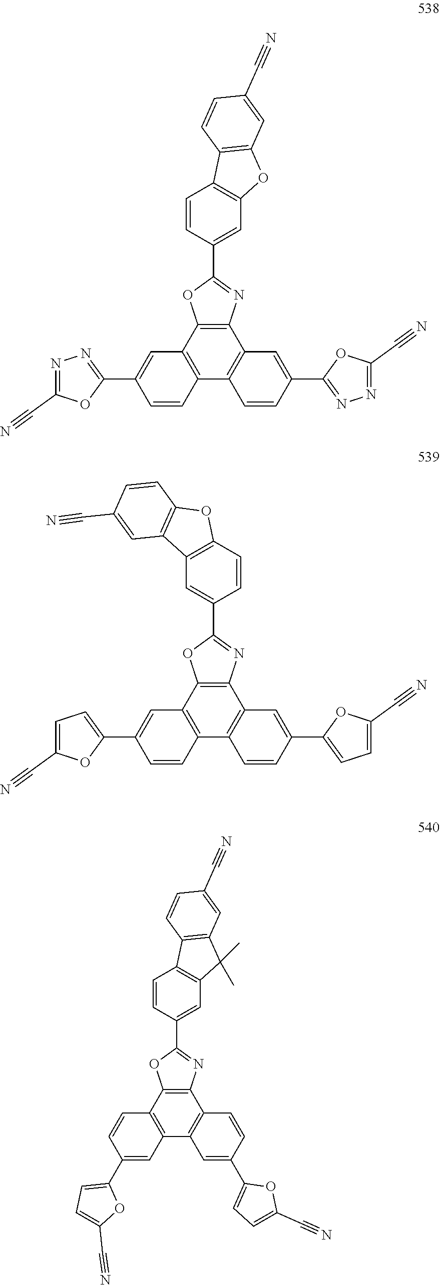 [SMILES: N#Cc1ccc2c(c1)oc1cc(-c3nc4c5cc(-c6nnc(C#N)o6)ccc5c5ccc(-c6nnc(C#N)o6)cc5c4o3)ccc12 ; N#Cc1ccc2oc3ccc(-c4nc5c6cc(-c7ccc(C#N)o7)ccc6c6ccc(-c7ccc(C#N)o7)cc6c5o4)cc3c2c1 ; CC1(C)c2cc(C#N)ccc2-c2ccc(-c3nc4c5ccc(-c6ccc(C#N)o6)cc5c5cc(-c6ccc(C#N)o6)ccc5c4o3)cc21]

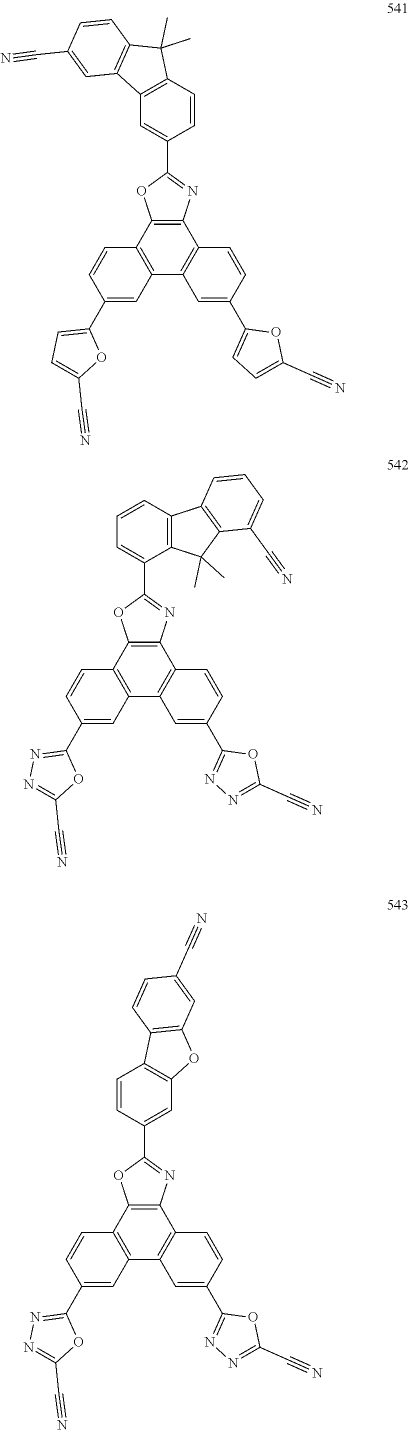 [SMILES: CC1(C)c2ccc(C#N)cc2-c2cc(-c3nc4c5ccc(-c6ccc(C#N)o6)cc5c5cc(-c6ccc(C#N)o6)ccc5c4o3)ccc21 ; CC1(C)c2c(C#N)cccc2-c2cccc(-c3nc4c5ccc(-c6nnc(C#N)o6)cc5c5cc(-c6nnc(C#N)o6)ccc5c4o3)c21 ; N#Cc1ccc2c(c1)oc1cc(-c3nc4c5ccc(-c6nnc(C#N)o6)cc5c5cc(-c6nnc(C#N)o6)ccc5c4o3)ccc12]

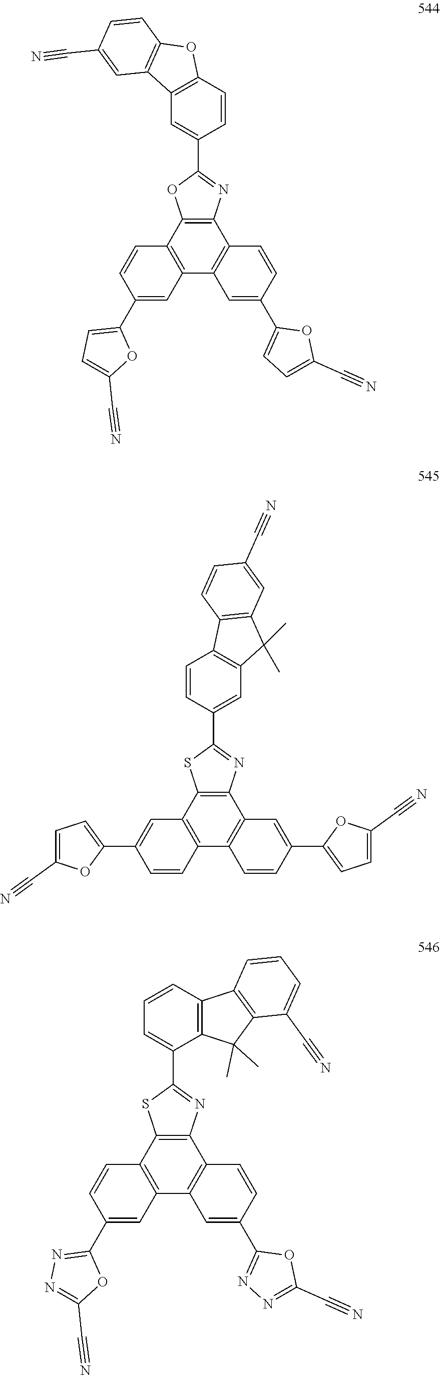 [SMILES: N#Cc1ccc2oc3ccc(-c4nc5c6ccc(-c7ccc(C#N)o7)cc6c6cc(-c7ccc(C#N)o7)ccc6c5o4)cc3c2c1 ; CC1(C)c2cc(C#N)ccc2-c2ccc(-c3nc4c5cc(-c6ccc(C#N)o6)ccc5c5ccc(-c6ccc(C#N)o6)cc5c4s3)cc21 ; CC1(C)c2c(C#N)cccc2-c2cccc(-c3nc4c5ccc(-c6nnc(C#N)o6)cc5c5cc(-c6nnc(C#N)o6)ccc5c4s3)c21]

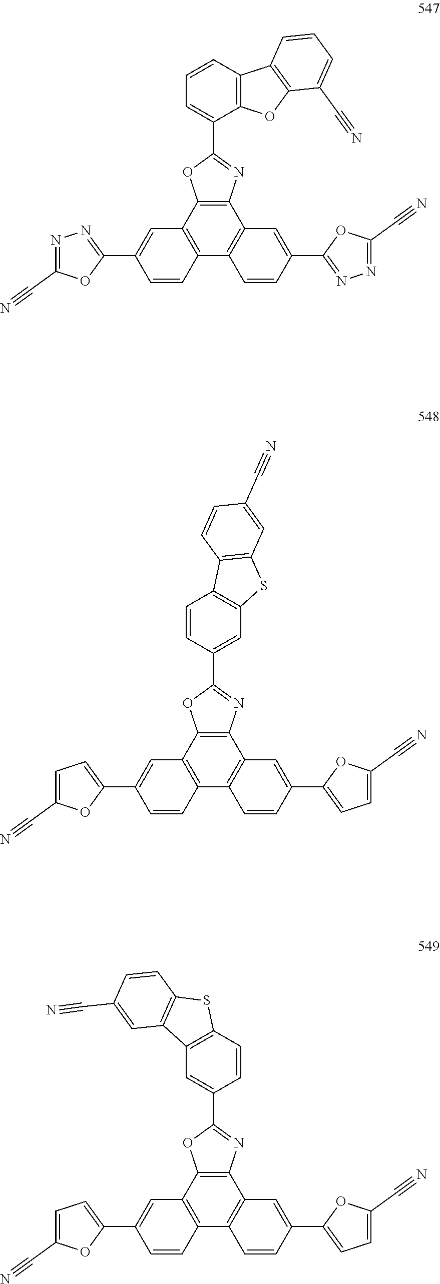 [SMILES: N#Cc1nnc(-c2ccc3c4ccc(-c5nnc(C#N)o5)cc4c4oc(-c5cccc6c5oc5c(C#N)cccc56)nc4c3c2)o1 ; N#Cc1ccc2c(c1)sc1cc(-c3nc4c5cc(-c6ccc(C#N)o6)ccc5c5ccc(-c6ccc(C#N)o6)cc5c4o3)ccc12 ; N#Cc1ccc2sc3ccc(-c4nc5c6cc(-c7ccc(C#N)o7)ccc6c6ccc(-c7ccc(C#N)o7)cc6c5o4)cc3c2c1]

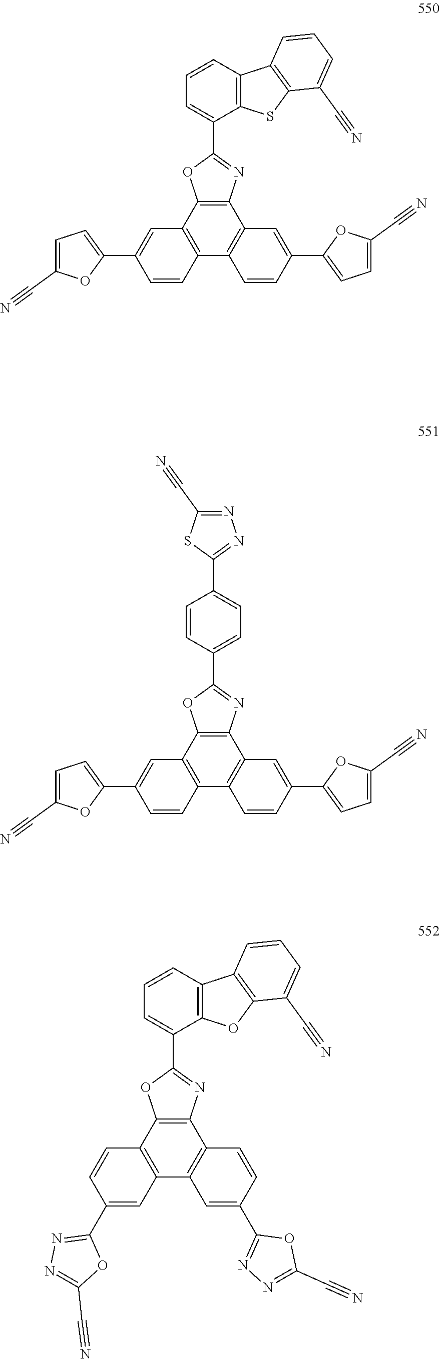 [SMILES: N#Cc1ccc(-c2ccc3c4ccc(-c5ccc(C#N)o5)cc4c4oc(-c5cccc6c5sc5c(C#N)cccc56)nc4c3c2)o1 ; N#Cc1ccc(-c2ccc3c4ccc(-c5ccc(C#N)o5)cc4c4oc(-c5ccc(-c6nnc(C#N)s6)cc5)nc4c3c2)o1 ; N#Cc1nnc(-c2ccc3c(c2)c2cc(-c4nnc(C#N)o4)ccc2c2oc(-c4cccc5c4oc4c(C#N)cccc45)nc32)o1]

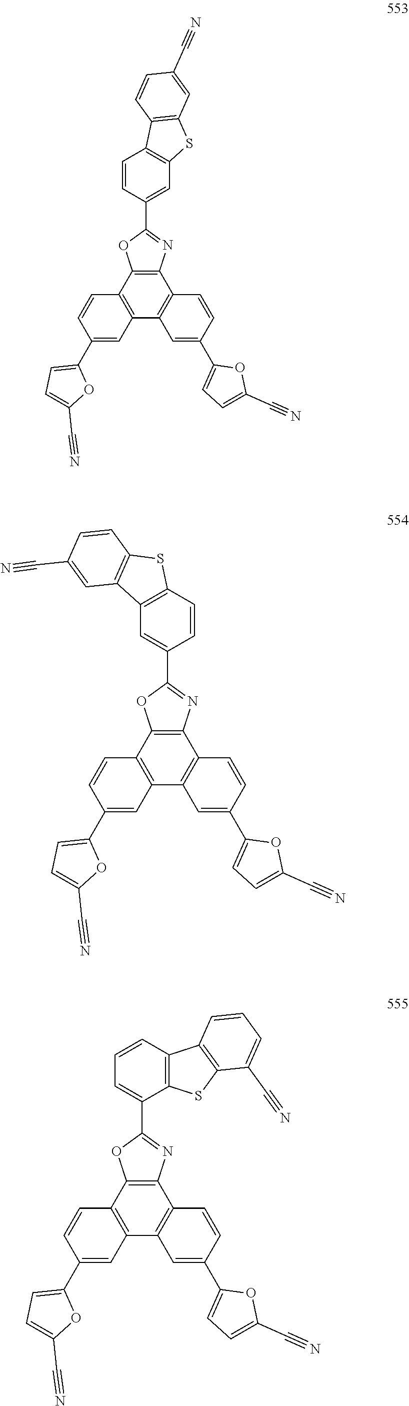 [SMILES: N#Cc1ccc2c(c1)sc1cc(-c3nc4c5ccc(-c6ccc(C#N)o6)cc5c5cc(-c6ccc(C#N)o6)ccc5c4o3)ccc12 ; N#Cc1ccc2sc3ccc(-c4nc5c6ccc(-c7ccc(C#N)o7)cc6c6cc(-c7ccc(C#N)o7)ccc6c5o4)cc3c2c1 ; N#Cc1ccc(-c2ccc3c(c2)c2cc(-c4ccc(C#N)o4)ccc2c2oc(-c4cccc5c4sc4c(C#N)cccc45)nc32)o1]

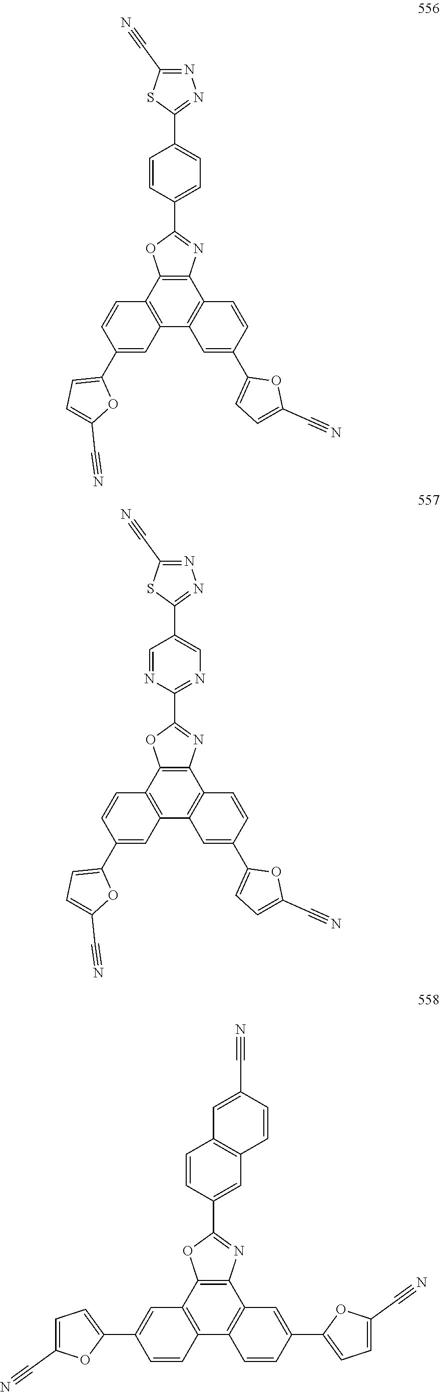 [SMILES: N#Cc1ccc(-c2ccc3c(c2)c2cc(-c4ccc(C#N)o4)ccc2c2oc(-c4ccc(-c5nnc(C#N)s5)cc4)nc32)o1 ; N#Cc1ccc(-c2ccc3c(c2)c2cc(-c4ccc(C#N)o4)ccc2c2oc(-c4ncc(-c5nnc(C#N)s5)cn4)nc32)o1 ; N#Cc1ccc2cc(-c3nc4c5cc(-c6ccc(C#N)o6)ccc5c5ccc(-c6ccc(C#N)o6)cc5c4o3)ccc2c1]

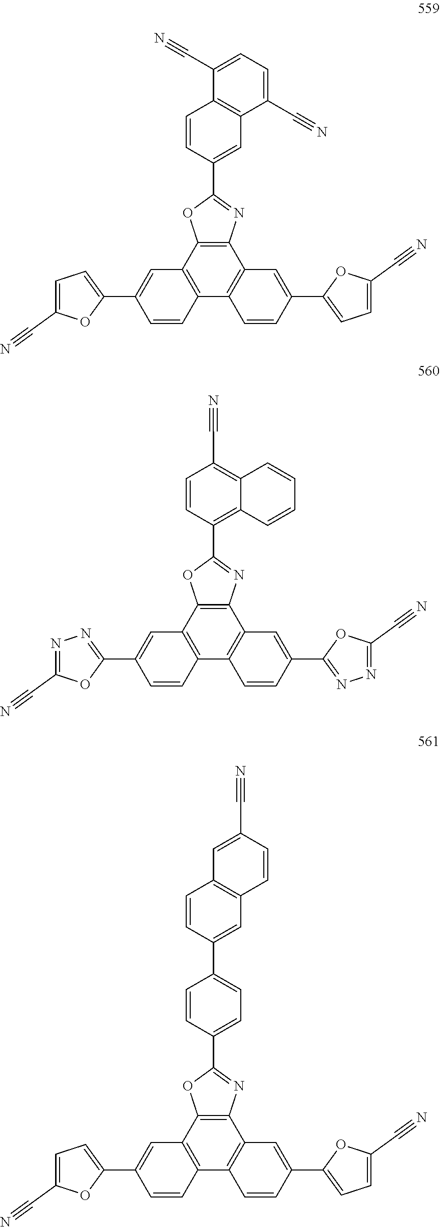 [SMILES: N#Cc1ccc(-c2ccc3c4ccc(-c5ccc(C#N)o5)cc4c4oc(-c5ccc6c(C#N)ccc(C#N)c6c5)nc4c3c2)o1 ; N#Cc1nnc(-c2ccc3c4ccc(-c5nnc(C#N)o5)cc4c4oc(-c5ccc(C#N)c6ccccc56)nc4c3c2)o1 ; N#Cc1ccc2cc(-c3ccc(-c4nc5c6cc(-c7ccc(C#N)o7)ccc6c6ccc(-c7ccc(C#N)o7)cc6c5o4)cc3)ccc2c1]

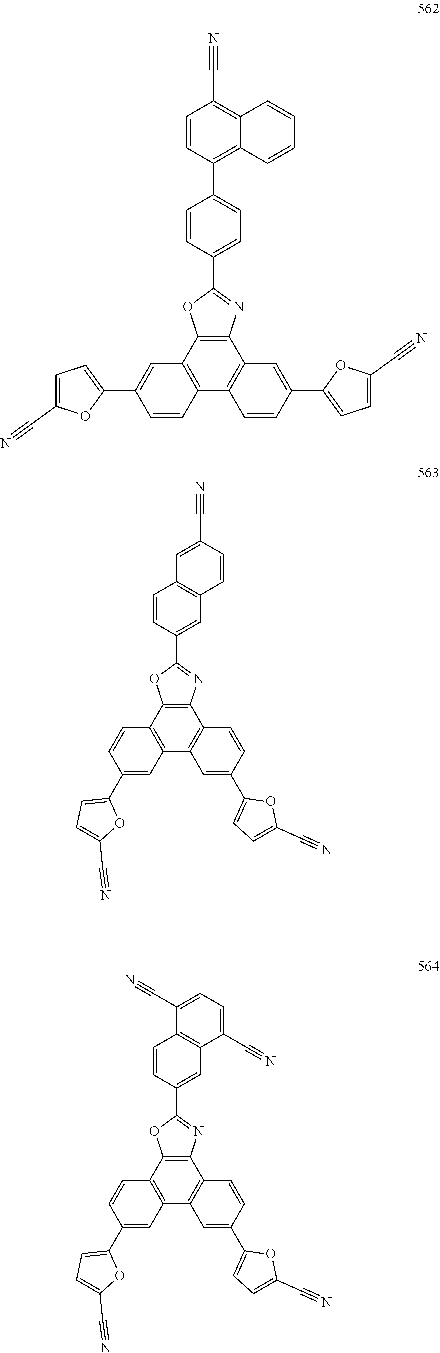 [SMILES: N#Cc1ccc(-c2ccc3c4ccc(-c5ccc(C#N)o5)cc4c4oc(-c5ccc(-c6ccc(C#N)c7ccccc67)cc5)nc4c3c2)o1 ; N#Cc1ccc2cc(-c3nc4c5ccc(-c6ccc(C#N)o6)cc5c5cc(-c6ccc(C#N)o6)ccc5c4o3)ccc2c1 ; N#Cc1ccc(-c2ccc3c(c2)c2cc(-c4ccc(C#N)o4)ccc2c2oc(-c4ccc5c(C#N)ccc(C#N)c5c4)nc32)o1]

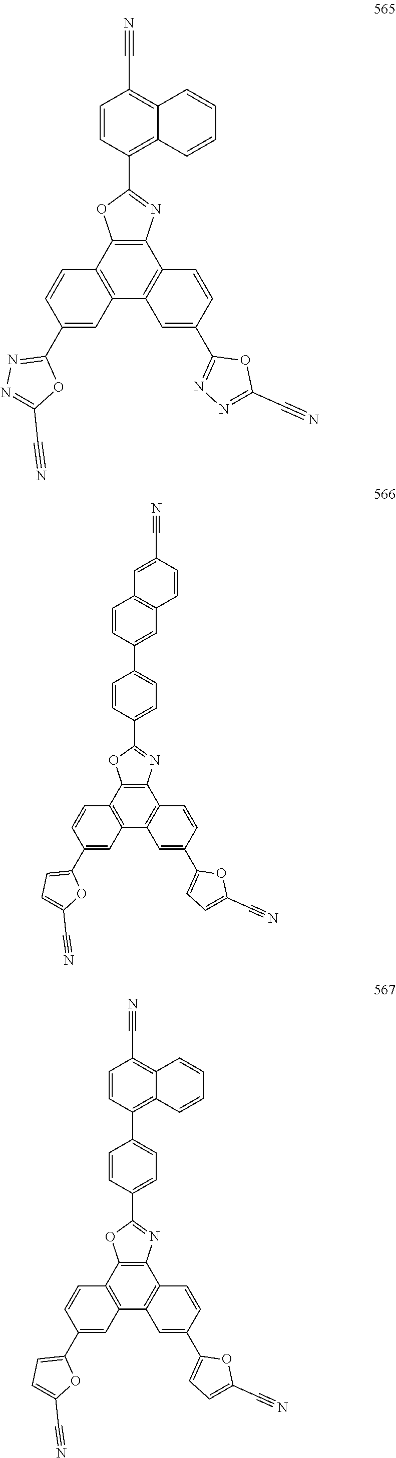 [SMILES: N#Cc1nnc(-c2ccc3c(c2)c2cc(-c4nnc(C#N)o4)ccc2c2oc(-c4ccc(C#N)c5ccccc45)nc32)o1 ; N#Cc1ccc2cc(-c3ccc(-c4nc5c6ccc(-c7ccc(C#N)o7)cc6c6cc(-c7ccc(C#N)o7)ccc6c5o4)cc3)ccc2c1 ; N#Cc1ccc(-c2ccc3c(c2)c2cc(-c4ccc(C#N)o4)ccc2c2oc(-c4ccc(-c5ccc(C#N)c6ccccc56)cc4)nc32)o1]

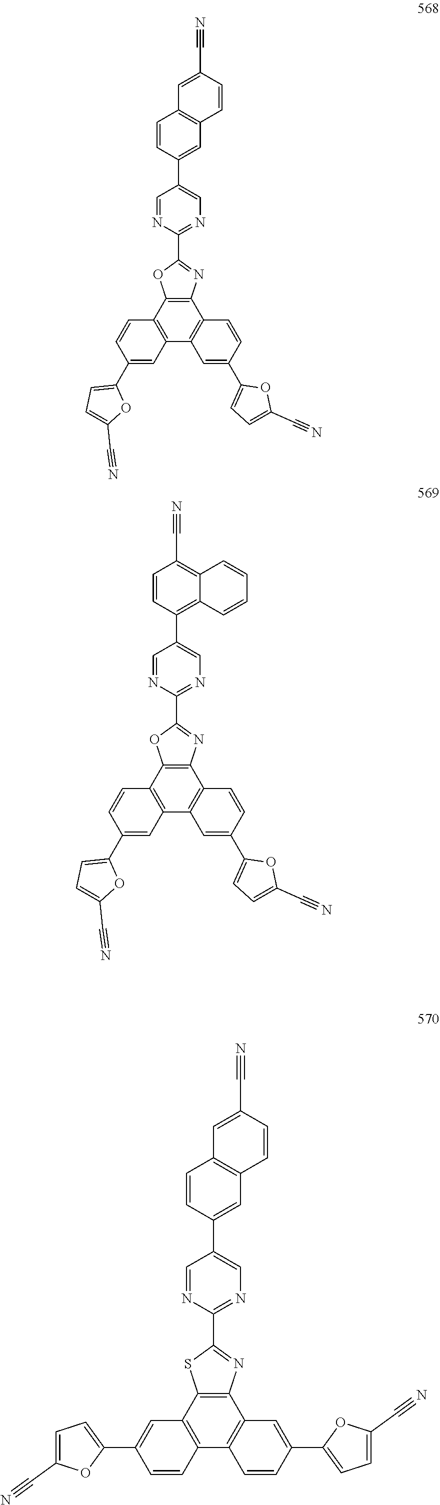 [SMILES: N#Cc1ccc2cc(-c3cnc(-c4nc5c6ccc(-c7ccc(C#N)o7)cc6c6cc(-c7ccc(C#N)o7)ccc6c5o4)nc3)ccc2c1 ; N#Cc1ccc(-c2ccc3c(c2)c2cc(-c4ccc(C#N)o4)ccc2c2oc(-c4ncc(-c5ccc(C#N)c6ccccc56)cn4)nc32)o1 ; N#Cc1ccc2cc(-c3cnc(-c4nc5c6cc(-c7ccc(C#N)o7)ccc6c6ccc(-c7ccc(C#N)o7)cc6c5s4)nc3)ccc2c1]

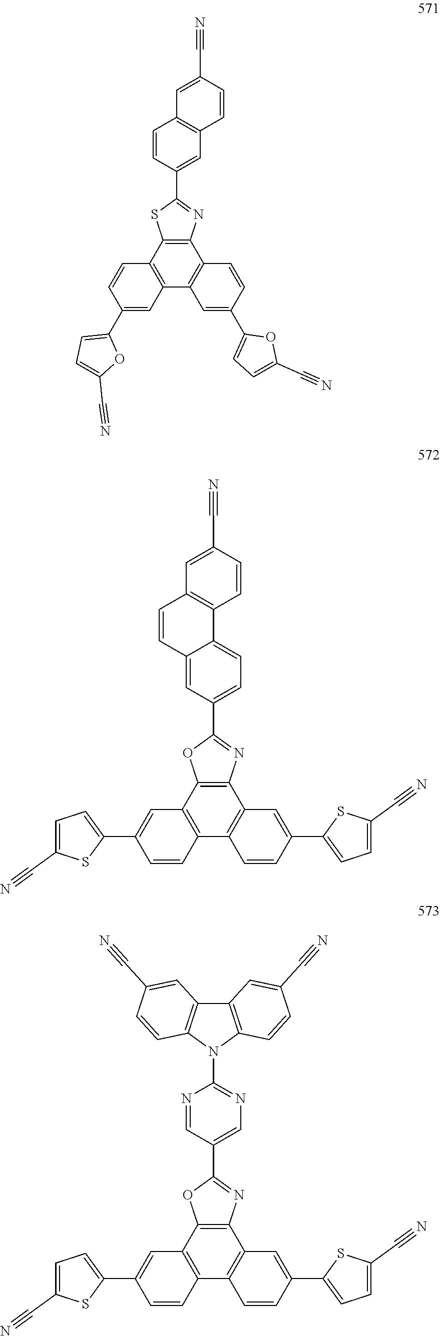 [SMILES: N#Cc1ccc2cc(-c3nc4c5ccc(-c6ccc(C#N)o6)cc5c5cc(-c6ccc(C#N)o6)ccc5c4s3)ccc2c1 ; N#Cc1ccc2c(ccc3cc(-c4nc5c6cc(-c7ccc(C#N)s7)ccc6c6ccc(-c7ccc(C#N)s7)cc6c5o4)ccc32)c1 ; N#Cc1ccc2c(c1)c1cc(C#N)ccc1n2-c1ncc(-c2nc3c4cc(-c5ccc(C#N)s5)ccc4c4ccc(-c5ccc(C#N)s5)cc4c3o2)cn1]

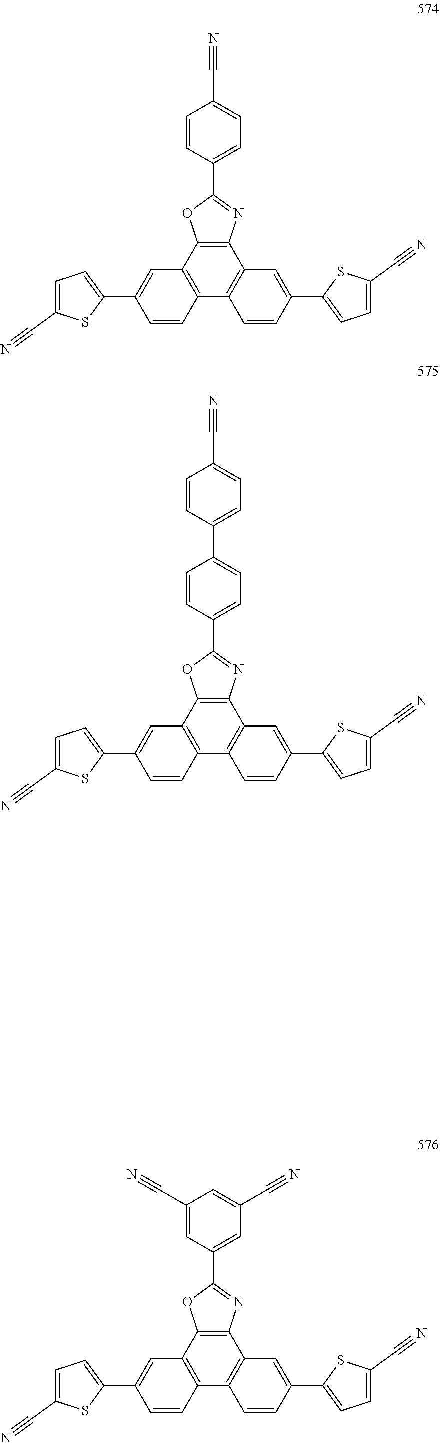 [SMILES: N#Cc1ccc(-c2nc3c4cc(-c5ccc(C#N)s5)ccc4c4ccc(-c5ccc(C#N)s5)cc4c3o2)cc1 ; N#Cc1ccc(-c2ccc(-c3nc4c5cc(-c6ccc(C#N)s6)ccc5c5ccc(-c6ccc(C#N)s6)cc5c4o3)cc2)cc1 ; N#Cc1cc(C#N)cc(-c2nc3c4cc(-c5ccc(C#N)s5)ccc4c4ccc(-c5ccc(C#N)s5)cc4c3o2)c1]

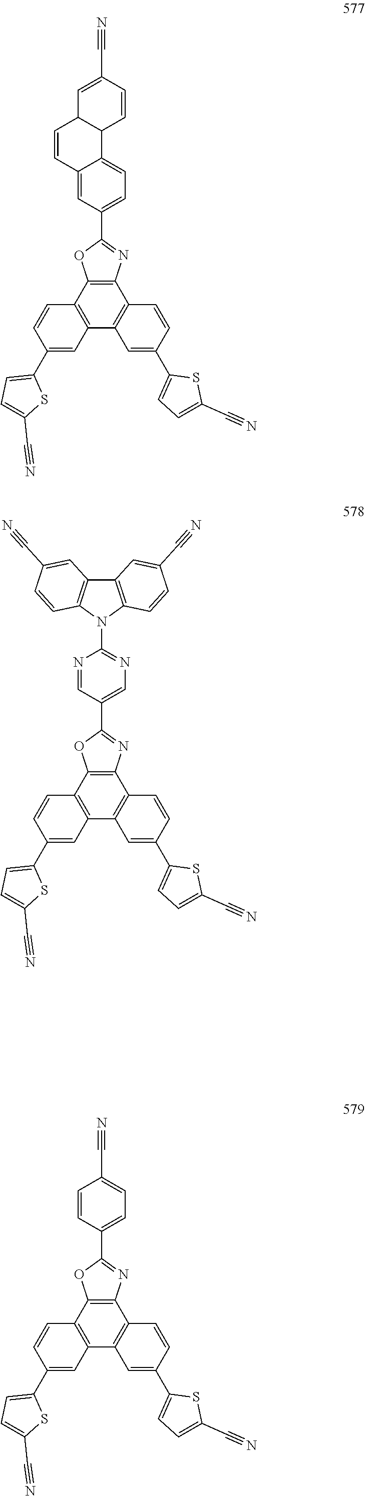 [SMILES: N#CC1=CC2C=Cc3cc(-c4nc5c6ccc(-c7ccc(C#N)s7)cc6c6cc(-c7ccc(C#N)s7)ccc6c5o4)ccc3C2C=C1 ; N#Cc1ccc2c(c1)c1cc(C#N)ccc1n2-c1ncc(-c2nc3c4ccc(-c5ccc(C#N)s5)cc4c4cc(-c5ccc(C#N)s5)ccc4c3o2)cn1 ; N#Cc1ccc(-c2nc3c4ccc(-c5ccc(C#N)s5)cc4c4cc(-c5ccc(C#N)s5)ccc4c3o2)cc1]

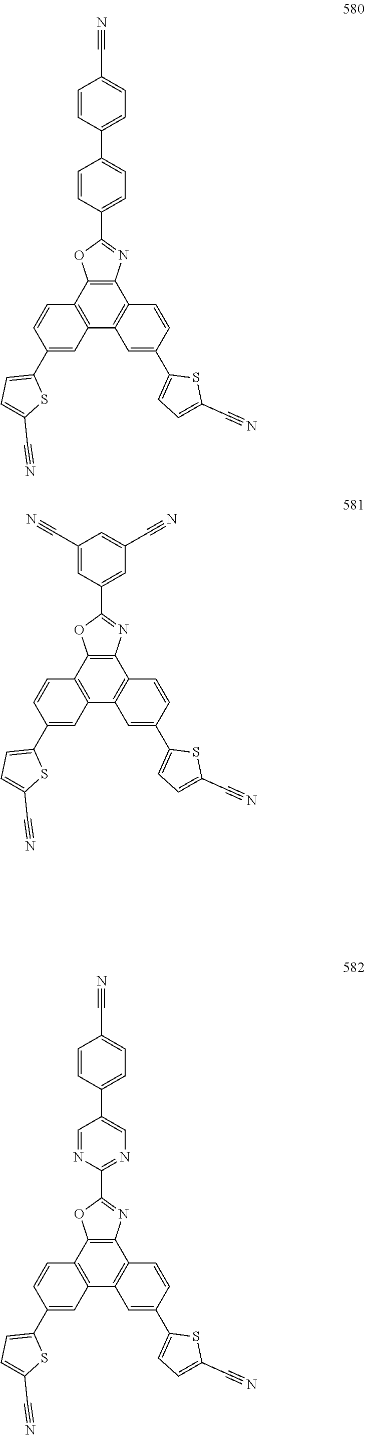 [SMILES: N#Cc1ccc(-c2ccc(-c3nc4c5ccc(-c6ccc(C#N)s6)cc5c5cc(-c6ccc(C#N)s6)ccc5c4o3)cc2)cc1 ; N#Cc1cc(C#N)cc(-c2nc3c4ccc(-c5ccc(C#N)s5)cc4c4cc(-c5ccc(C#N)s5)ccc4c3o2)c1 ; N#Cc1ccc(-c2cnc(-c3nc4c5ccc(-c6ccc(C#N)s6)cc5c5cc(-c6ccc(C#N)s6)ccc5c4o3)nc2)cc1]

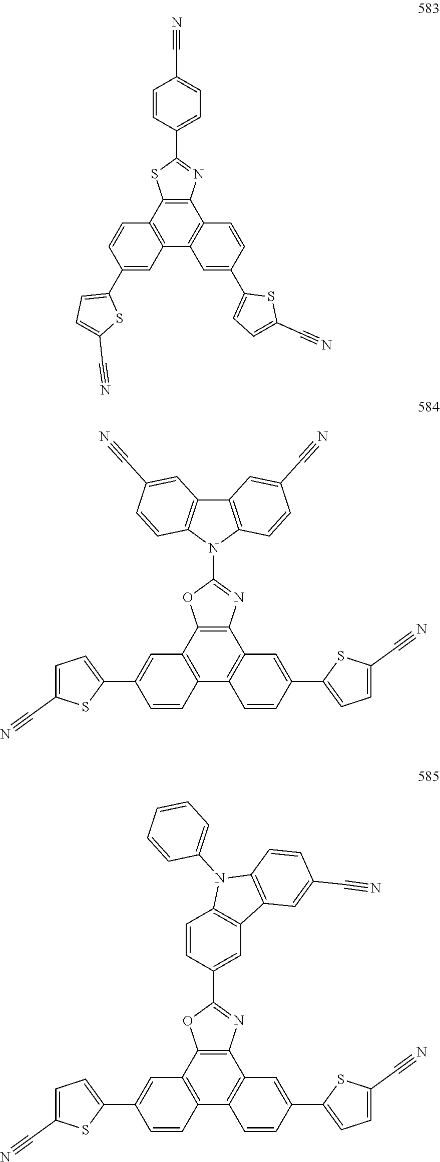 [SMILES: N#Cc1ccc(-c2nc3c4ccc(-c5ccc(C#N)s5)cc4c4cc(-c5ccc(C#N)s5)ccc4c3s2)cc1 ; N#Cc1ccc2c(c1)c1cc(C#N)ccc1n2-c1nc2c3cc(-c4ccc(C#N)s4)ccc3c3ccc(-c4ccc(C#N)s4)cc3c2o1 ; N#Cc1ccc2c(c1)c1cc(-c3nc4c5cc(-c6ccc(C#N)s6)ccc5c5ccc(-c6ccc(C#N)s6)cc5c4o3)ccc1n2-c1ccccc1]

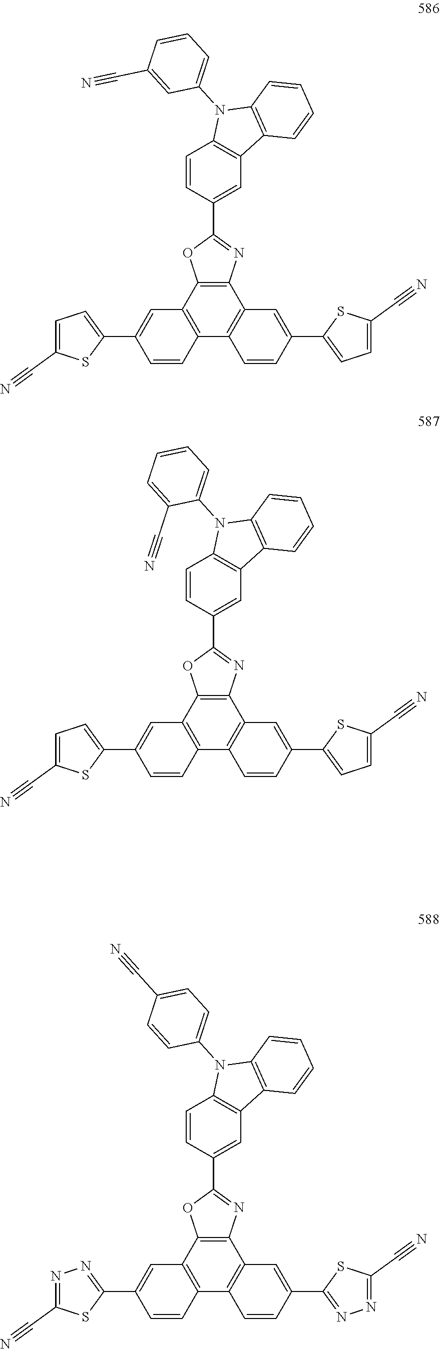 [SMILES: N#Cc1cccc(-n2c3ccccc3c3cc(-c4nc5c6cc(-c7ccc(C#N)s7)ccc6c6ccc(-c7ccc(C#N)s7)cc6c5o4)ccc32)c1 ; N#Cc1ccc(-c2ccc3c4ccc(-c5ccc(C#N)s5)cc4c4oc(-c5ccc6c(c5)c5ccccc5n6-c5ccccc5C#N)nc4c3c2)s1 ; N#Cc1ccc(-n2c3ccccc3c3cc(-c4nc5c6cc(-c7nnc(C#N)s7)ccc6c6ccc(-c7nnc(C#N)s7)cc6c5o4)ccc32)cc1]

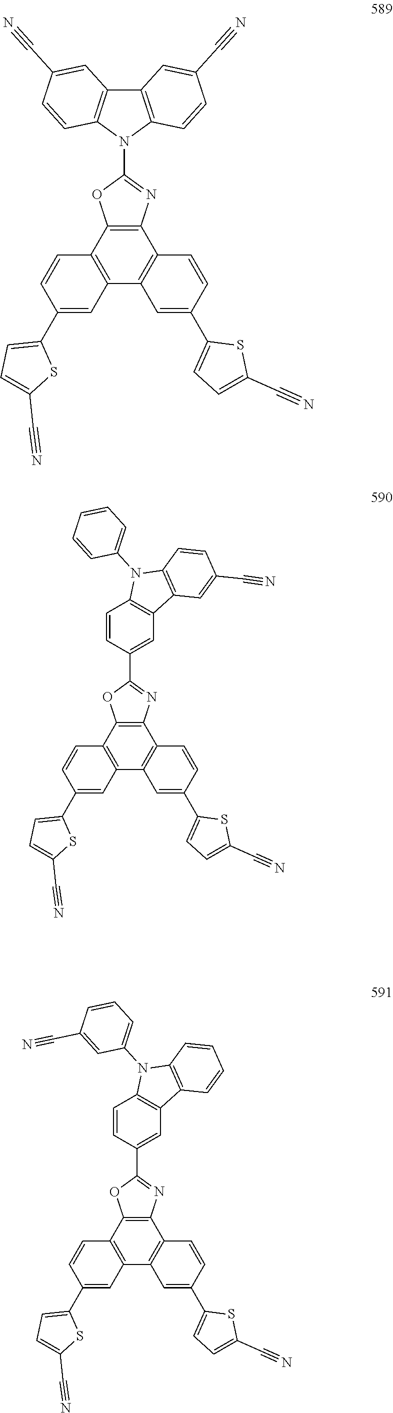 [SMILES: N#Cc1ccc2c(c1)c1cc(C#N)ccc1n2-c1nc2c3ccc(-c4ccc(C#N)s4)cc3c3cc(-c4ccc(C#N)s4)ccc3c2o1 ; N#Cc1ccc2c(c1)c1cc(-c3nc4c5ccc(-c6ccc(C#N)s6)cc5c5cc(-c6ccc(C#N)s6)ccc5c4o3)ccc1n2-c1ccccc1 ; N#Cc1cccc(-n2c3ccccc3c3cc(-c4nc5c6ccc(-c7ccc(C#N)s7)cc6c6cc(-c7ccc(C#N)s7)ccc6c5o4)ccc32)c1]

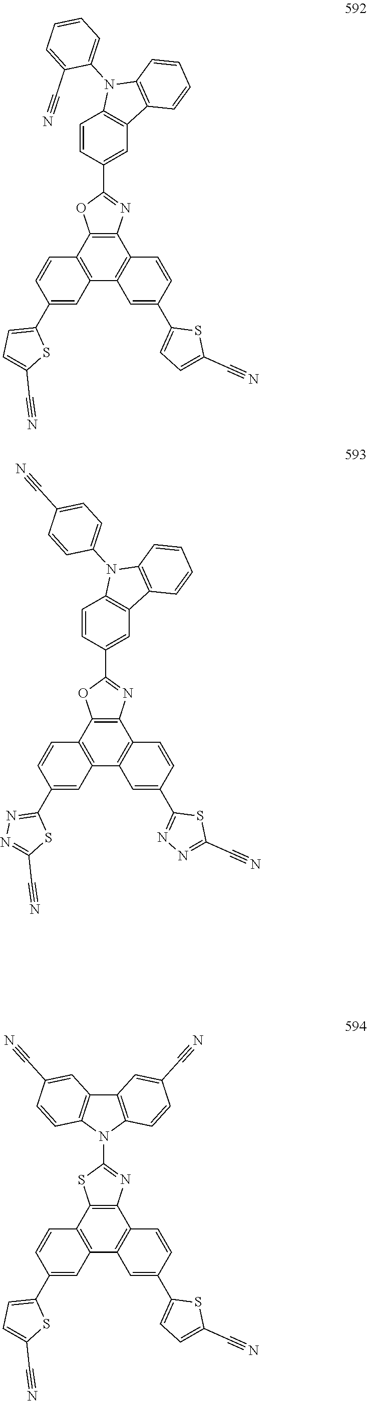 [SMILES: N#Cc1ccc(-c2ccc3c(c2)c2cc(-c4ccc(C#N)s4)ccc2c2oc(-c4ccc5c(c4)c4ccccc4n5-c4ccccc4C#N)nc32)s1 ; N#Cc1ccc(-n2c3ccccc3c3cc(-c4nc5c6ccc(-c7nnc(C#N)s7)cc6c6cc(-c7nnc(C#N)s7)ccc6c5o4)ccc32)cc1 ; N#Cc1ccc2c(c1)c1cc(C#N)ccc1n2-c1nc2c3ccc(-c4ccc(C#N)s4)cc3c3cc(-c4ccc(C#N)s4)ccc3c2s1]

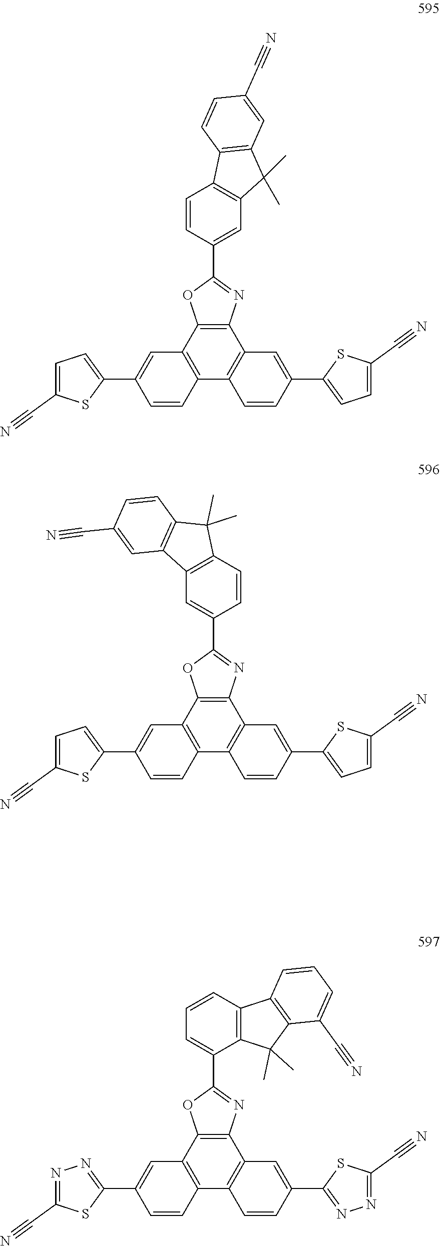 [SMILES: CC1(C)c2cc(C#N)ccc2-c2ccc(-c3nc4c5cc(-c6ccc(C#N)s6)ccc5c5ccc(-c6ccc(C#N)s6)cc5c4o3)cc21 ; CC1(C)c2ccc(C#N)cc2-c2cc(-c3nc4c5cc(-c6ccc(C#N)s6)ccc5c5ccc(-c6ccc(C#N)s6)cc5c4o3)ccc21 ; CC1(C)c2c(C#N)cccc2-c2cccc(-c3nc4c5cc(-c6nnc(C#N)s6)ccc5c5ccc(-c6nnc(C#N)s6)cc5c4o3)c21]

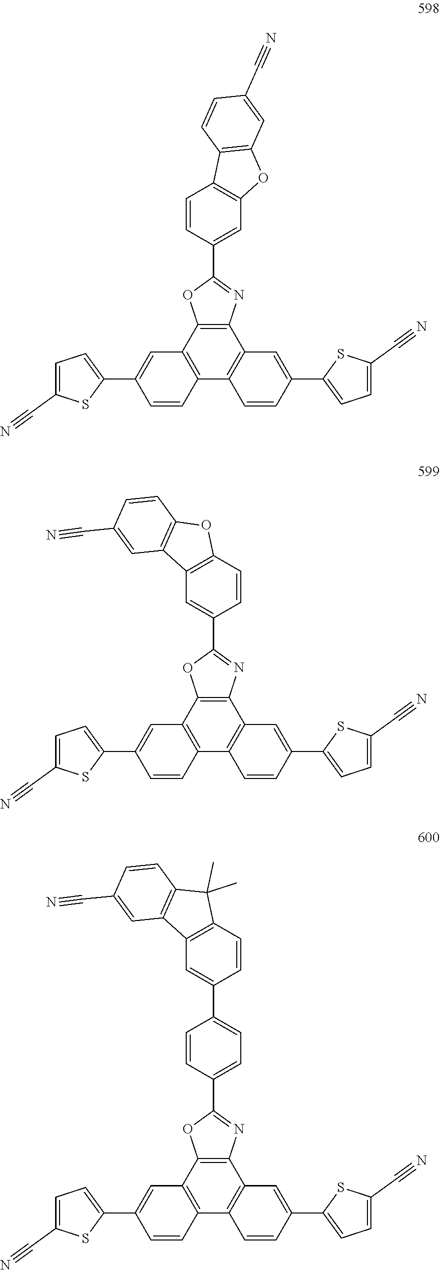 [SMILES: N#Cc1ccc2c(c1)oc1cc(-c3nc4c5cc(-c6ccc(C#N)s6)ccc5c5ccc(-c6ccc(C#N)s6)cc5c4o3)ccc12 ; N#Cc1ccc2oc3ccc(-c4nc5c6cc(-c7ccc(C#N)s7)ccc6c6ccc(-c7ccc(C#N)s7)cc6c5o4)cc3c2c1 ; CC1(C)c2ccc(C#N)cc2-c2cc(-c3ccc(-c4nc5c6cc(-c7ccc(C#N)s7)ccc6c6ccc(-c7ccc(C#N)s7)cc6c5o4)cc3)ccc21]

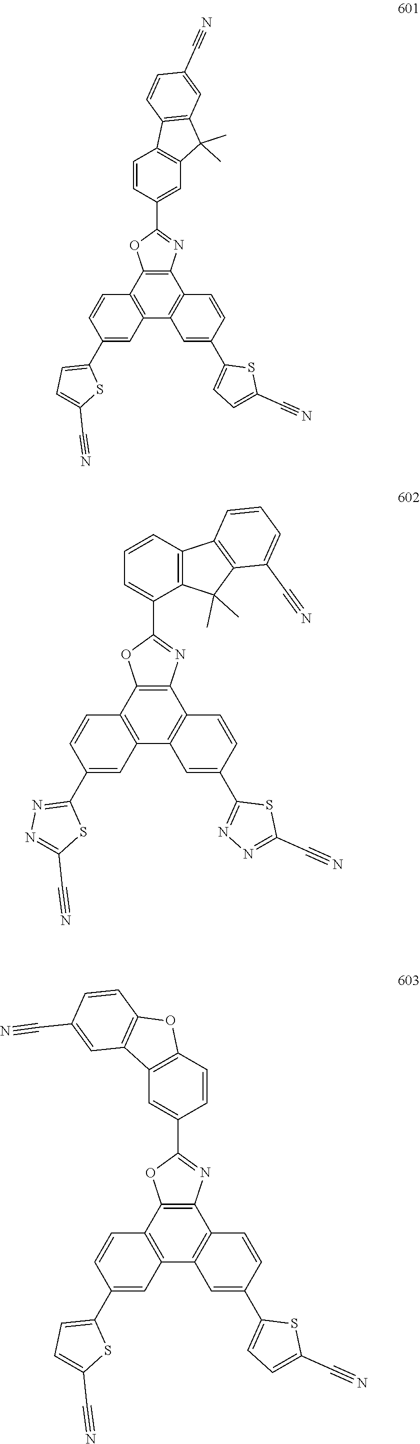 [SMILES: CC1(C)c2cc(C#N)ccc2-c2ccc(-c3nc4c5ccc(-c6ccc(C#N)s6)cc5c5cc(-c6ccc(C#N)s6)ccc5c4o3)cc21 ; CC1(C)c2c(C#N)cccc2-c2cccc(-c3nc4c5ccc(-c6nnc(C#N)s6)cc5c5cc(-c6nnc(C#N)s6)ccc5c4o3)c21 ; N#Cc1ccc2oc3ccc(-c4nc5c6ccc(-c7ccc(C#N)s7)cc6c6cc(-c7ccc(C#N)s7)ccc6c5o4)cc3c2c1]

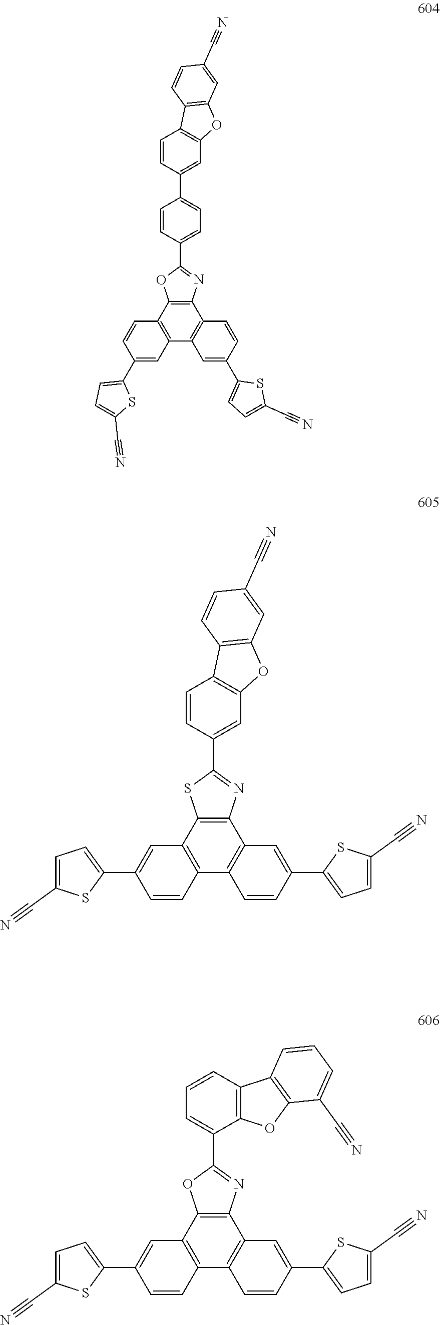 [SMILES: N#Cc1ccc2c(c1)oc1cc(-c3ccc(-c4nc5c6ccc(-c7ccc(C#N)s7)cc6c6cc(-c7ccc(C#N)s7)ccc6c5o4)cc3)ccc12 ; N#Cc1ccc2c(c1)oc1cc(-c3nc4c5cc(-c6ccc(C#N)s6)ccc5c5ccc(-c6ccc(C#N)s6)cc5c4s3)ccc12 ; N#Cc1ccc(-c2ccc3c4ccc(-c5ccc(C#N)s5)cc4c4oc(-c5cccc6c5oc5c(C#N)cccc56)nc4c3c2)s1]

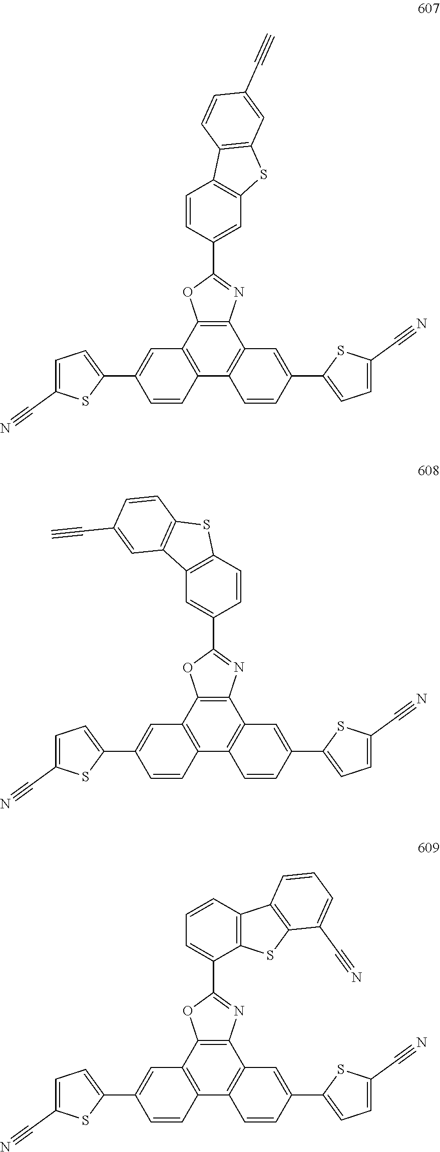 [SMILES: C#Cc1ccc2c(c1)sc1cc(-c3nc4c5cc(-c6ccc(C#N)s6)ccc5c5ccc(-c6ccc(C#N)s6)cc5c4o3)ccc12 ; C#Cc1ccc2sc3ccc(-c4nc5c6cc(-c7ccc(C#N)s7)ccc6c6ccc(-c7ccc(C#N)s7)cc6c5o4)cc3c2c1 ; N#Cc1ccc(-c2ccc3c4ccc(-c5ccc(C#N)s5)cc4c4oc(-c5cccc6c5sc5c(C#N)cccc56)nc4c3c2)s1]

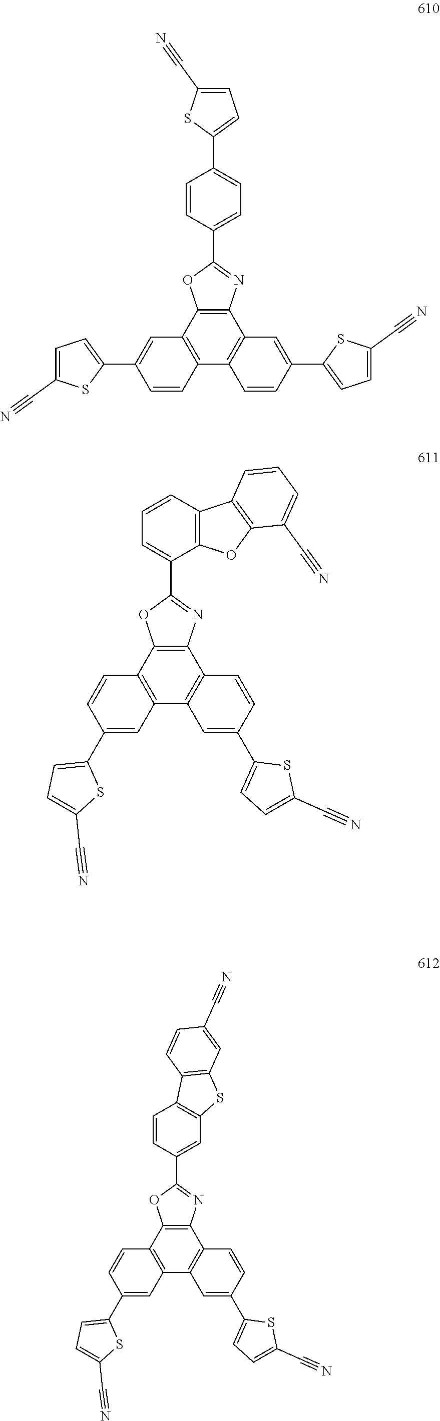 [SMILES: N#Cc1ccc(-c2ccc(-c3nc4c5cc(-c6ccc(C#N)s6)ccc5c5ccc(-c6ccc(C#N)s6)cc5c4o3)cc2)s1 ; N#Cc1ccc(-c2ccc3c(c2)c2cc(-c4ccc(C#N)s4)ccc2c2oc(-c4cccc5c4oc4c(C#N)cccc45)nc32)s1 ; N#Cc1ccc2c(c1)sc1cc(-c3nc4c5ccc(-c6ccc(C#N)s6)cc5c5cc(-c6ccc(C#N)s6)ccc5c4o3)ccc12]

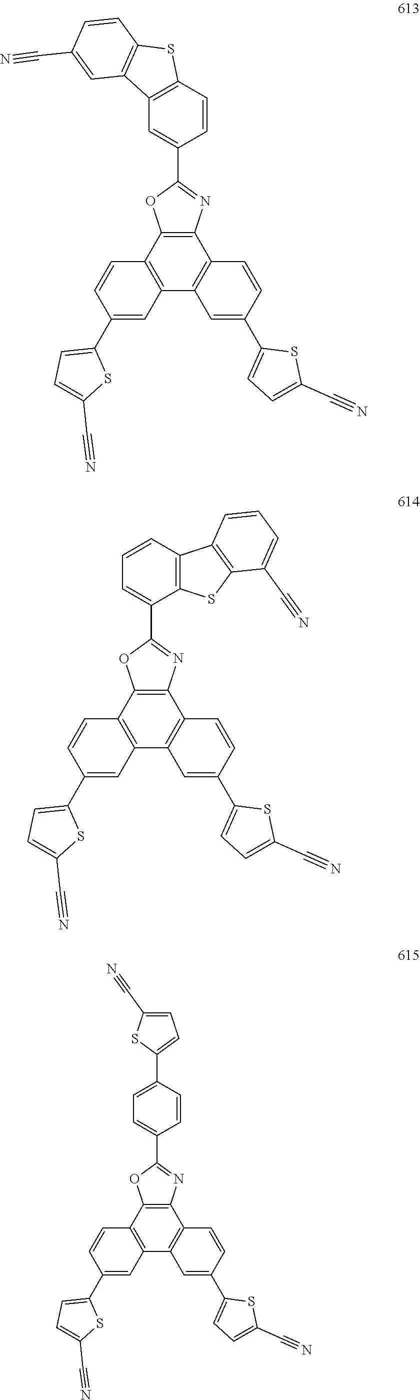 [SMILES: N#Cc1ccc2sc3ccc(-c4nc5c6ccc(-c7ccc(C#N)s7)cc6c6cc(-c7ccc(C#N)s7)ccc6c5o4)cc3c2c1 ; N#Cc1ccc(-c2ccc3c(c2)c2cc(-c4ccc(C#N)s4)ccc2c2oc(-c4cccc5c4sc4c(C#N)cccc45)nc32)s1 ; N#Cc1ccc(-c2ccc(-c3nc4c5ccc(-c6ccc(C#N)s6)cc5c5cc(-c6ccc(C#N)s6)ccc5c4o3)cc2)s1]

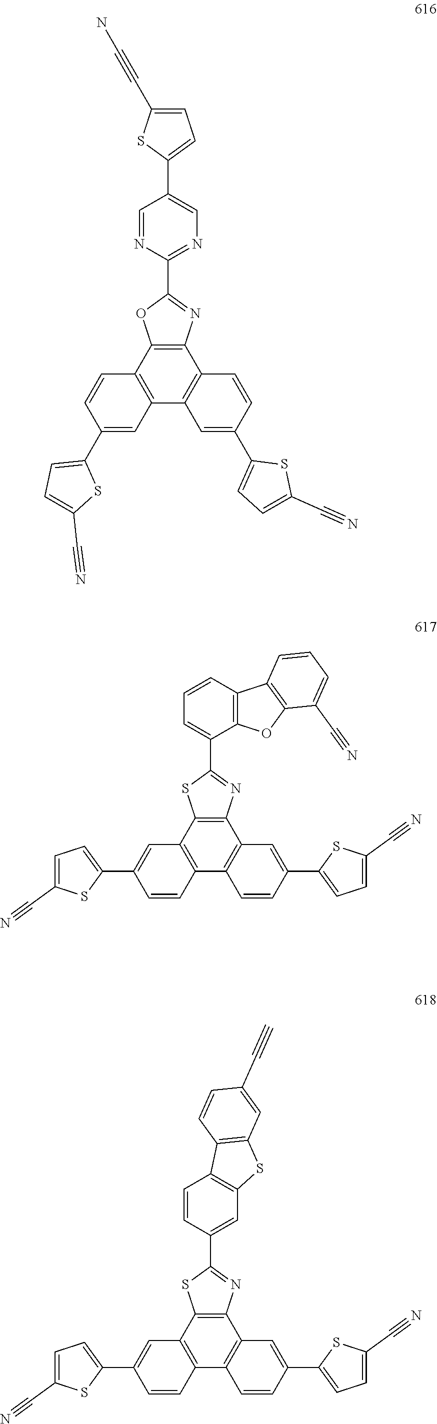 [SMILES: N#Cc1ccc(-c2ccc3c(c2)c2cc(-c4ccc(C#N)s4)ccc2c2oc(-c4ncc(-c5ccc(C#CN)s5)cn4)nc32)s1 ; N#Cc1ccc(-c2ccc3c4ccc(-c5ccc(C#N)s5)cc4c4sc(-c5cccc6c5oc5c(C#N)cccc56)nc4c3c2)s1 ; C#Cc1ccc2c(c1)sc1cc(-c3nc4c5cc(-c6ccc(C#N)s6)ccc5c5ccc(-c6ccc(C#N)s6)cc5c4s3)ccc12]

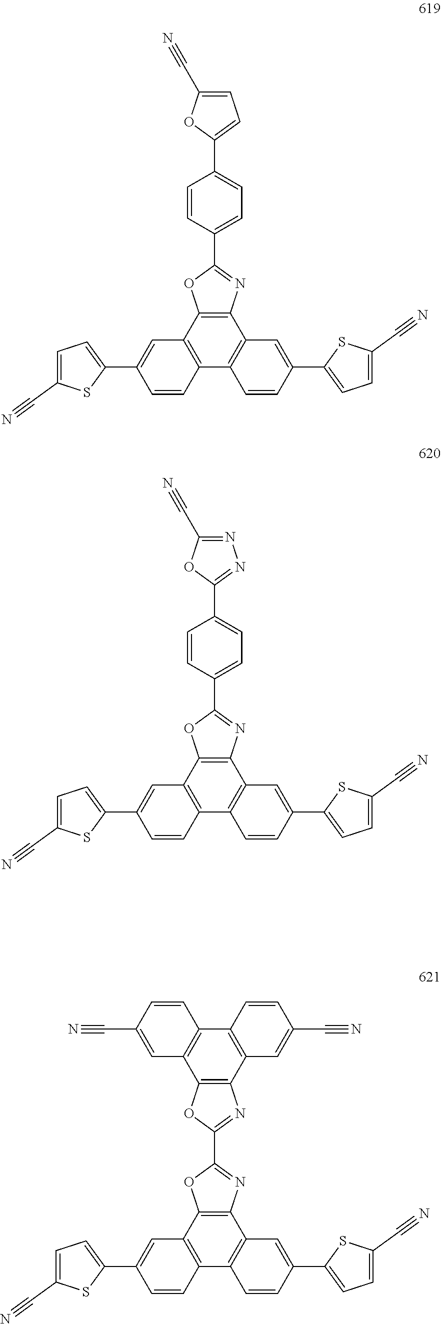 [SMILES: N#Cc1ccc(-c2ccc(-c3nc4c5cc(-c6ccc(C#N)s6)ccc5c5ccc(-c6ccc(C#N)s6)cc5c4o3)cc2)o1 ; N#Cc1nnc(-c2ccc(-c3nc4c5cc(-c6ccc(C#N)s6)ccc5c5ccc(-c6ccc(C#N)s6)cc5c4o3)cc2)o1 ; N#Cc1ccc2c3ccc(C#N)cc3c3oc(-c4nc5c6cc(-c7ccc(C#N)s7)ccc6c6ccc(-c7ccc(C#N)s7)cc6c5o4)nc3c2c1]

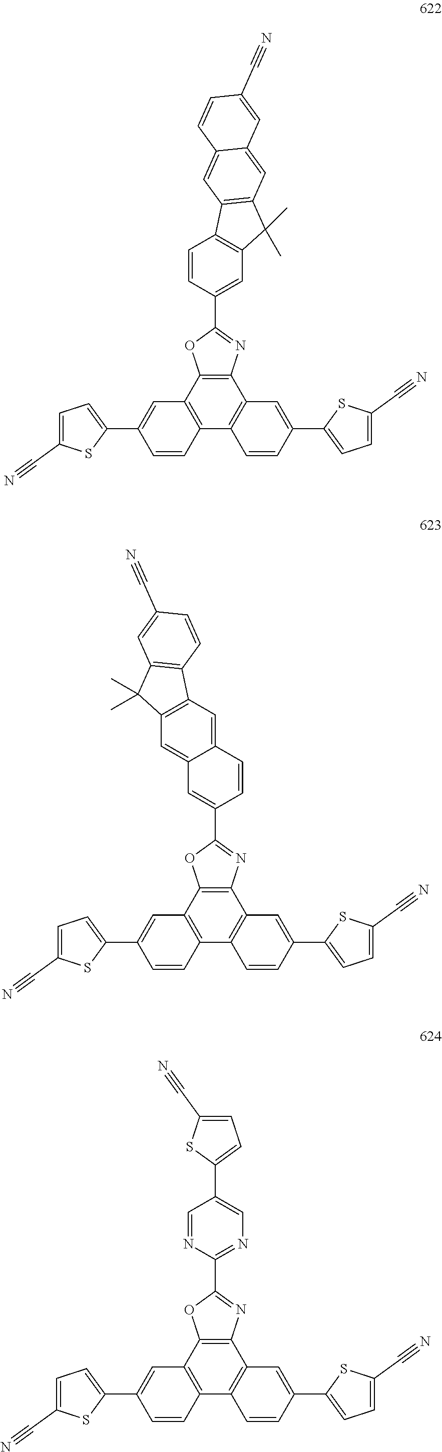 [SMILES: CC1(C)c2cc(-c3nc4c5cc(-c6ccc(C#N)s6)ccc5c5ccc(-c6ccc(C#N)s6)cc5c4o3)ccc2-c2cc3ccc(C#N)cc3cc21 ; CC1(C)c2cc(C#N)ccc2-c2cc3ccc(-c4nc5c6cc(-c7ccc(C#N)s7)ccc6c6ccc(-c7ccc(C#N)s7)cc6c5o4)cc3cc21 ; N#Cc1ccc(-c2cnc(-c3nc4c5cc(-c6ccc(C#N)s6)ccc5c5ccc(-c6ccc(C#N)s6)cc5c4o3)nc2)s1]

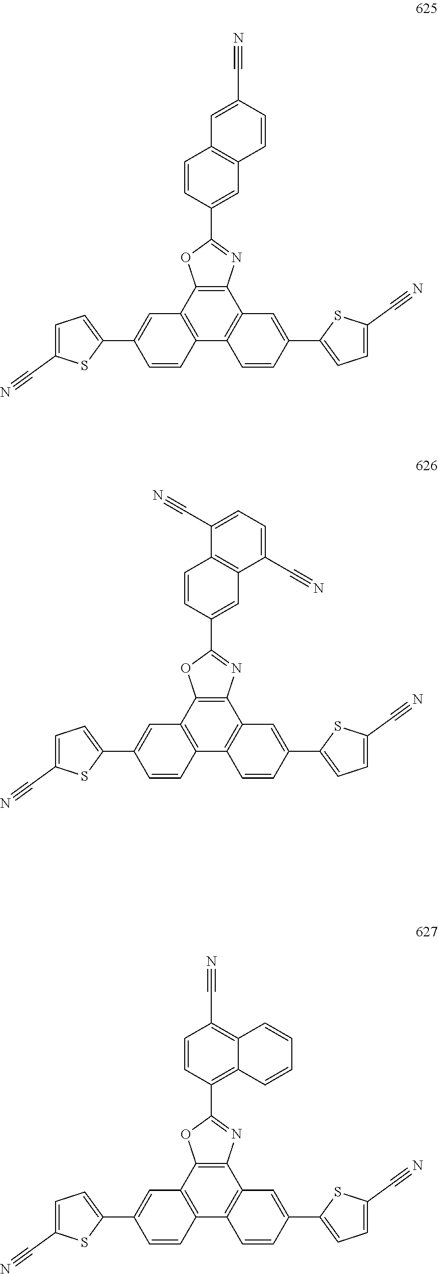 [SMILES: N#Cc1ccc2cc(-c3nc4c5cc(-c6ccc(C#N)s6)ccc5c5ccc(-c6ccc(C#N)s6)cc5c4o3)ccc2c1 ; N#Cc1ccc(-c2ccc3c4ccc(-c5ccc(C#N)s5)cc4c4oc(-c5ccc6c(C#N)ccc(C#N)c6c5)nc4c3c2)s1 ; N#Cc1ccc(-c2ccc3c4ccc(-c5ccc(C#N)s5)cc4c4oc(-c5ccc(C#N)c6ccccc56)nc4c3c2)s1]

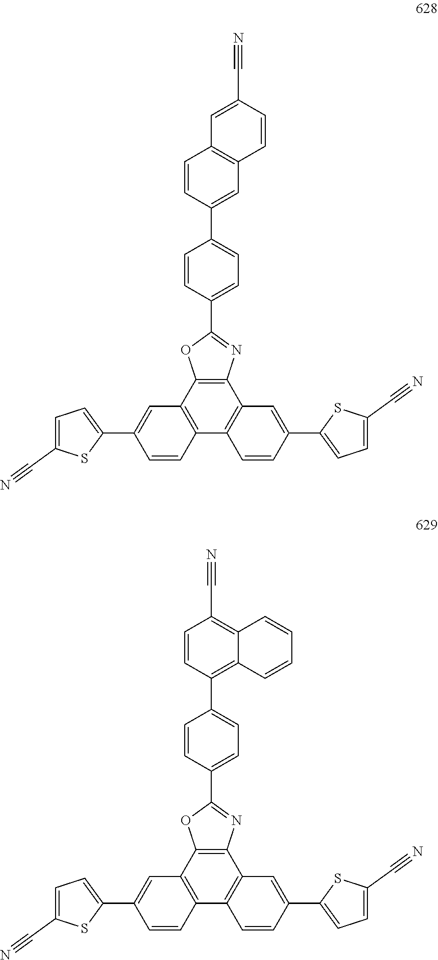 [SMILES: N#Cc1ccc2cc(-c3ccc(-c4nc5c6cc(-c7ccc(C#N)s7)ccc6c6ccc(-c7ccc(C#N)s7)cc6c5o4)cc3)ccc2c1 ; N#Cc1ccc(-c2ccc3c4ccc(-c5ccc(C#N)s5)cc4c4oc(-c5ccc(-c6ccc(C#N)c7ccccc67)cc5)nc4c3c2)s1]

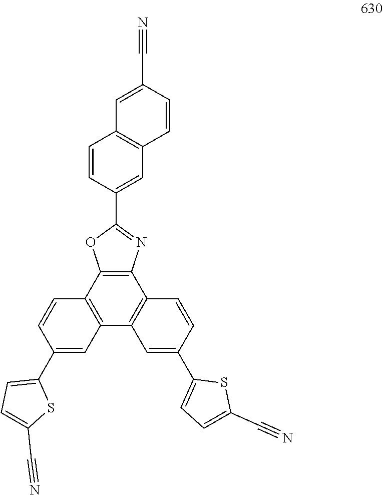 [SMILES: N#Cc1ccc2cc(-c3nc4c5ccc(-c6ccc(C#N)s6)cc5c5cc(-c6ccc(C#N)s6)ccc5c4o3)ccc2c1]

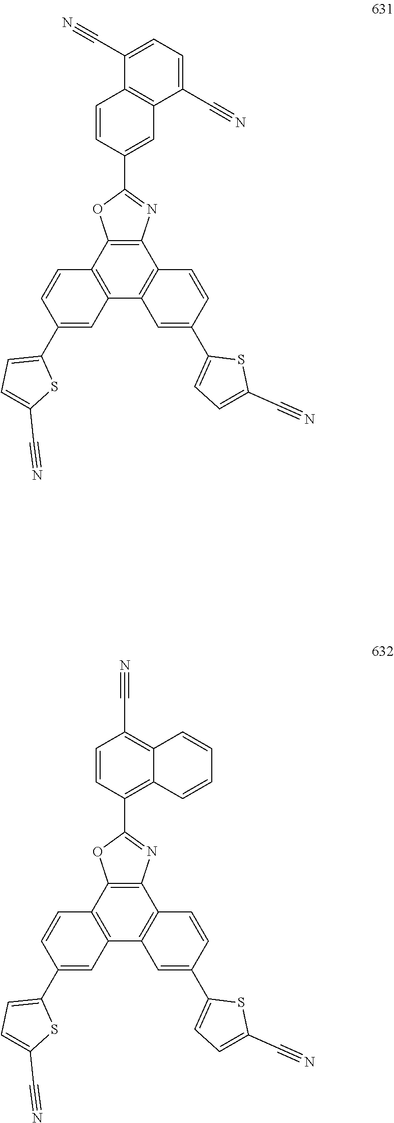 [SMILES: N#Cc1ccc(-c2ccc3c(c2)c2cc(-c4ccc(C#N)s4)ccc2c2oc(-c4ccc5c(C#N)ccc(C#N)c5c4)nc32)s1 ; N#Cc1ccc(-c2ccc3c(c2)c2cc(-c4ccc(C#N)s4)ccc2c2oc(-c4ccc(C#N)c5ccccc45)nc32)s1]

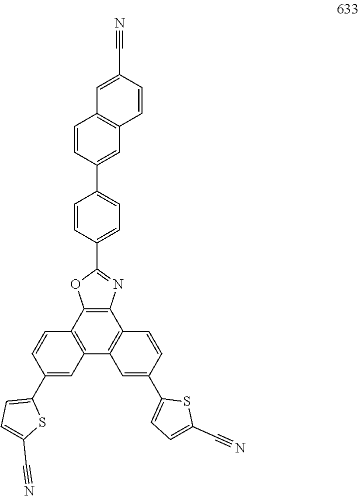 [SMILES: N#Cc1ccc2cc(-c3ccc(-c4nc5c6ccc(-c7ccc(C#N)s7)cc6c6cc(-c7ccc(C#N)s7)ccc6c5o4)cc3)ccc2c1]

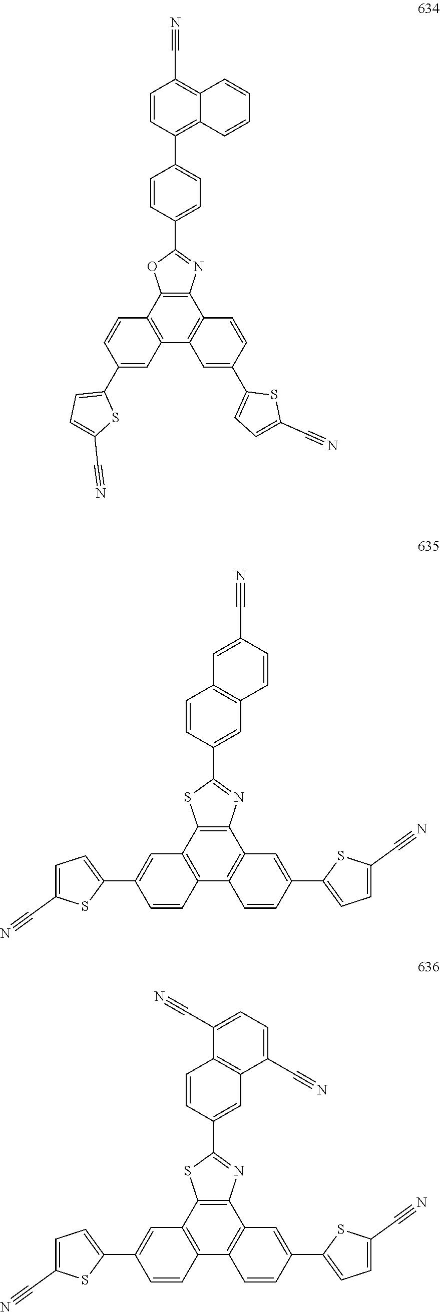 [SMILES: N#Cc1ccc(-c2ccc3c(c2)c2cc(-c4ccc(C#N)s4)ccc2c2oc(-c4ccc(-c5ccc(C#N)c6ccccc56)cc4)nc32)s1 ; N#Cc1ccc2cc(-c3nc4c5cc(-c6ccc(C#N)s6)ccc5c5ccc(-c6ccc(C#N)s6)cc5c4s3)ccc2c1 ; N#Cc1ccc(-c2ccc3c4ccc(-c5ccc(C#N)s5)cc4c4sc(-c5ccc6c(C#N)ccc(C#N)c6c5)nc4c3c2)s1]

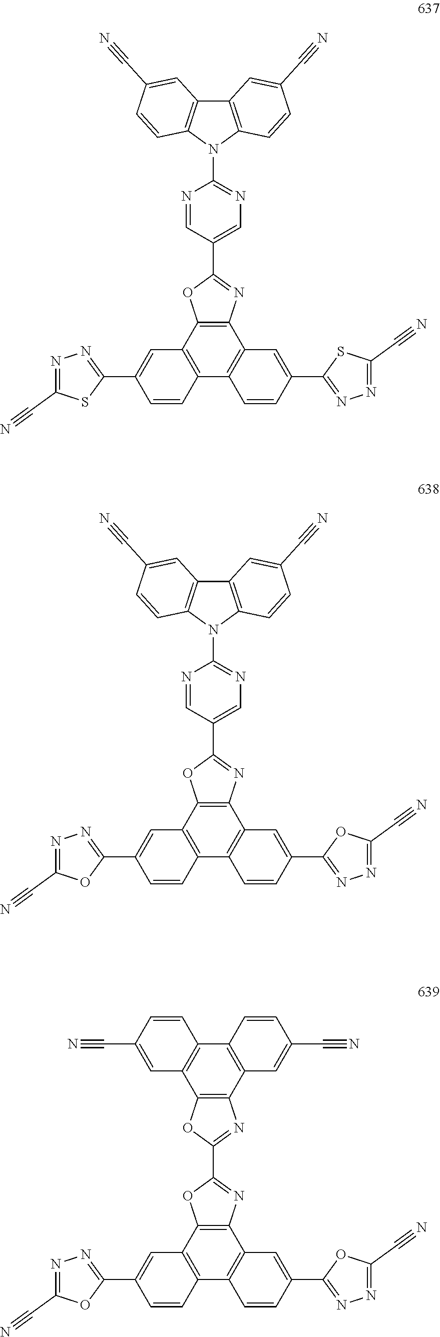 [SMILES: N#Cc1ccc2c(c1)c1cc(C#N)ccc1n2-c1ncc(-c2nc3c4cc(-c5nnc(C#N)s5)ccc4c4ccc(-c5nnc(C#N)s5)cc4c3o2)cn1 ; N#Cc1ccc2c(c1)c1cc(C#N)ccc1n2-c1ncc(-c2nc3c4cc(-c5nnc(C#N)o5)ccc4c4ccc(-c5nnc(C#N)o5)cc4c3o2)cn1 ; N#Cc1ccc2c3ccc(C#N)cc3c3oc(-c4nc5c6cc(-c7nnc(C#N)o7)ccc6c6ccc(-c7nnc(C#N)o7)cc6c5o4)nc3c2c1]

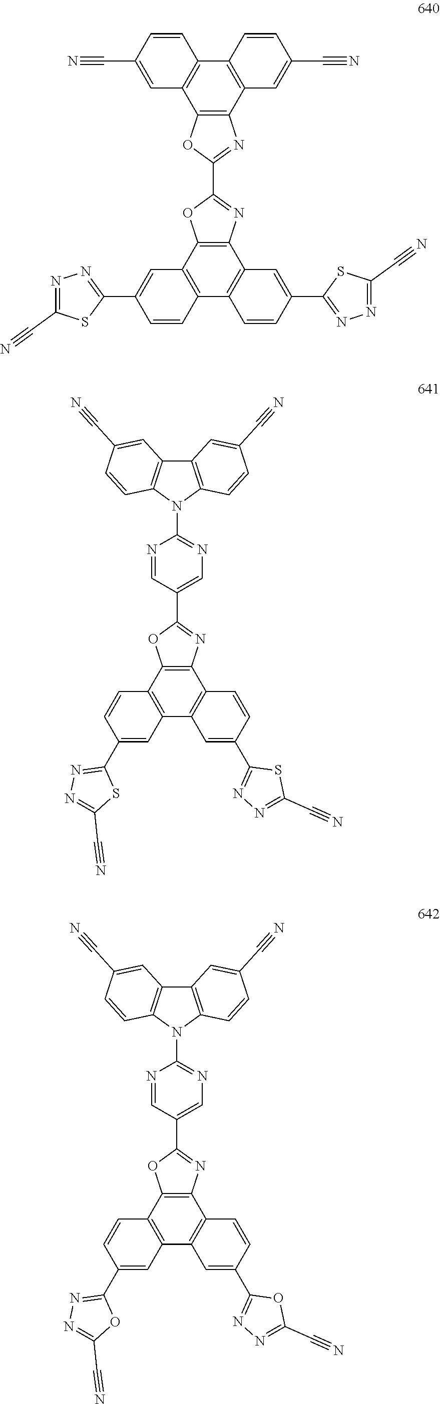 [SMILES: N#Cc1ccc2c3ccc(C#N)cc3c3oc(-c4nc5c6cc(-c7nnc(C#N)s7)ccc6c6ccc(-c7nnc(C#N)s7)cc6c5o4)nc3c2c1 ; N#Cc1ccc2c(c1)c1cc(C#N)ccc1n2-c1ncc(-c2nc3c4ccc(-c5nnc(C#N)s5)cc4c4cc(-c5nnc(C#N)s5)ccc4c3o2)cn1 ; N#Cc1ccc2c(c1)c1cc(C#N)ccc1n2-c1ncc(-c2nc3c4ccc(-c5nnc(C#N)o5)cc4c4cc(-c5nnc(C#N)o5)ccc4c3o2)cn1]

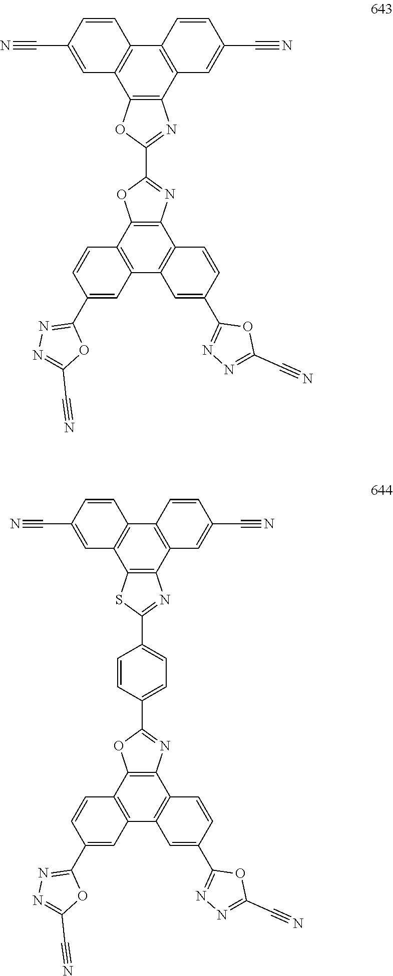 [SMILES: N#Cc1ccc2c3ccc(C#N)cc3c3oc(-c4nc5c6ccc(-c7nnc(C#N)o7)cc6c6cc(-c7nnc(C#N)o7)ccc6c5o4)nc3c2c1 ; N#Cc1ccc2c3ccc(C#N)cc3c3sc(-c4ccc(-c5nc6c7ccc(-c8nnc(C#N)o8)cc7c7cc(-c8nnc(C#N)o8)ccc7c6o5)cc4)nc3c2c1]

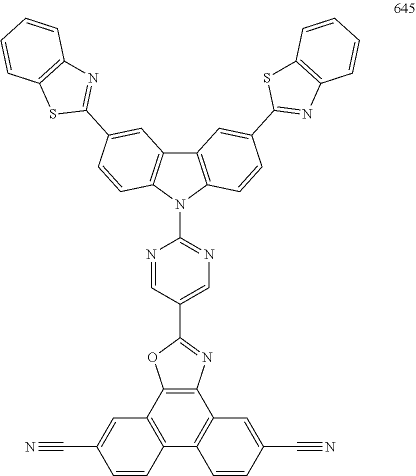 [SMILES: N#Cc1ccc2c3ccc(C#N)cc3c3oc(-c4cnc(-n5c6ccc(-c7nc8ccccc8s7)cc6c6cc(-c7nc8ccccc8s7)ccc65)nc4)nc3c2c1]

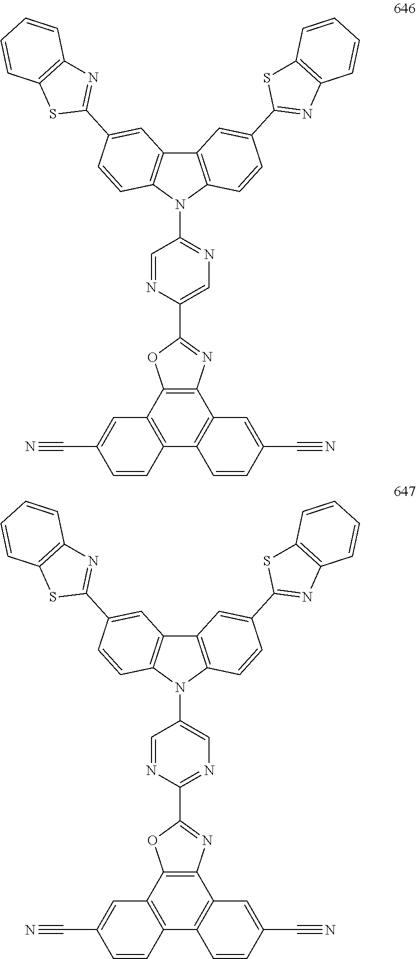 [SMILES: N#Cc1ccc2c3ccc(C#N)cc3c3oc(-c4cnc(-n5c6ccc(-c7nc8ccccc8s7)cc6c6cc(-c7nc8ccccc8s7)ccc65)cn4)nc3c2c1 ; N#Cc1ccc2c3ccc(C#N)cc3c3oc(-c4ncc(-n5c6ccc(-c7nc8ccccc8s7)cc6c6cc(-c7nc8ccccc8s7)ccc65)cn4)nc3c2c1]

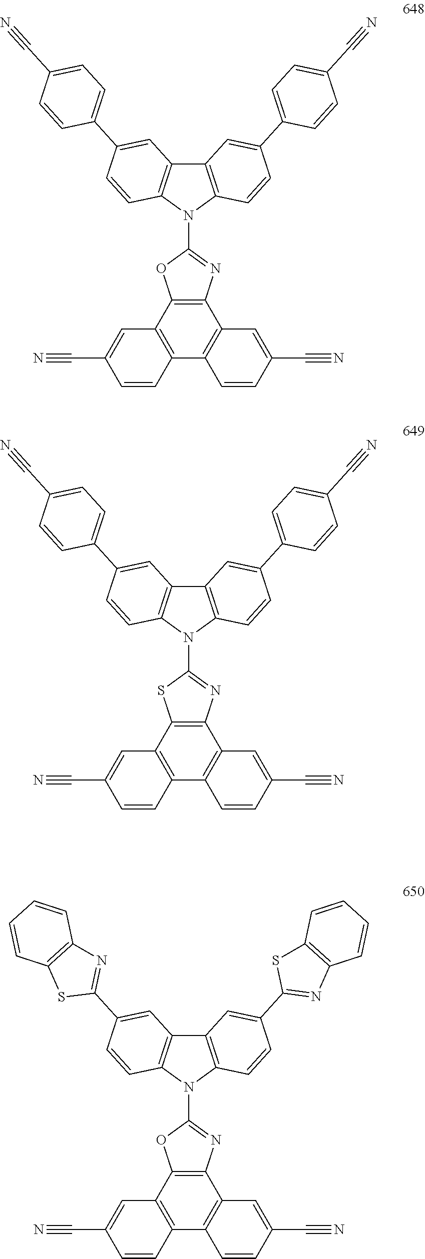 [SMILES: N#Cc1ccc(-c2ccc3c(c2)c2cc(-c4ccc(C#N)cc4)ccc2n3-c2nc3c4cc(C#N)ccc4c4ccc(C#N)cc4c3o2)cc1 ; N#Cc1ccc(-c2ccc3c(c2)c2cc(-c4ccc(C#N)cc4)ccc2n3-c2nc3c4cc(C#N)ccc4c4ccc(C#N)cc4c3s2)cc1 ; N#Cc1ccc2c3ccc(C#N)cc3c3oc(-n4c5ccc(-c6nc7ccccc7s6)cc5c5cc(-c6nc7ccccc7s6)ccc54)nc3c2c1]

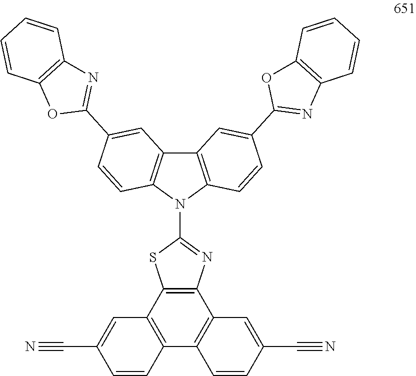 [SMILES: N#Cc1ccc2c3ccc(C#N)cc3c3sc(-n4c5ccc(-c6nc7ccccc7o6)cc5c5cc(-c6nc7ccccc7o6)ccc54)nc3c2c1]

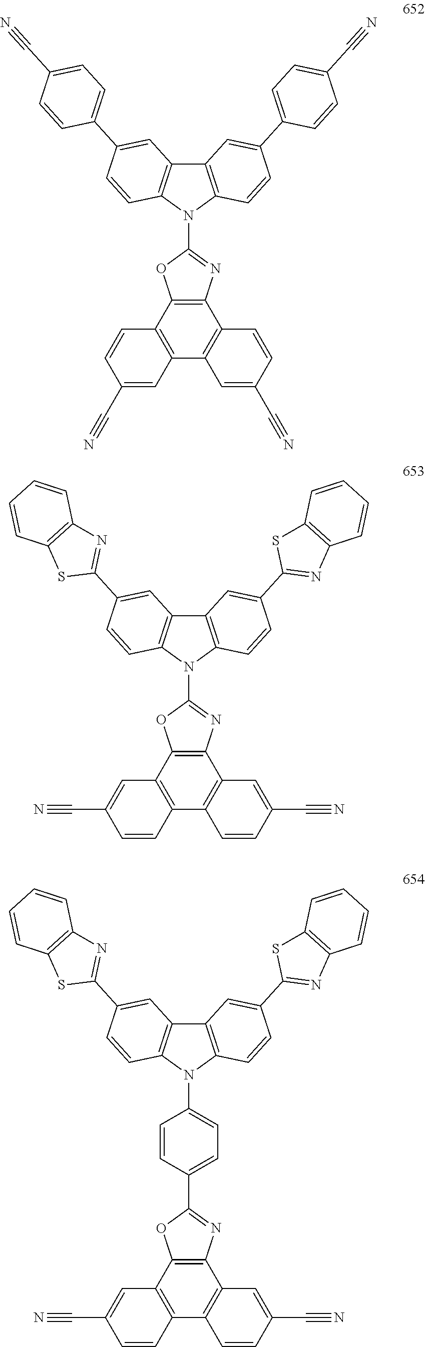 [SMILES: N#Cc1ccc(-c2ccc3c(c2)c2cc(-c4ccc(C#N)cc4)ccc2n3-c2nc3c4ccc(C#N)cc4c4cc(C#N)ccc4c3o2)cc1 ; N#Cc1ccc2c3ccc(C#N)cc3c3oc(-n4c5ccc(-c6nc7ccccc7s6)cc5c5cc(-c6nc7ccccc7s6)ccc54)nc3c2c1 ; N#Cc1ccc2c3ccc(C#N)cc3c3oc(-c4ccc(-n5c6ccc(-c7nc8ccccc8s7)cc6c6cc(-c7nc8ccccc8s7)ccc65)cc4)nc3c2c1]

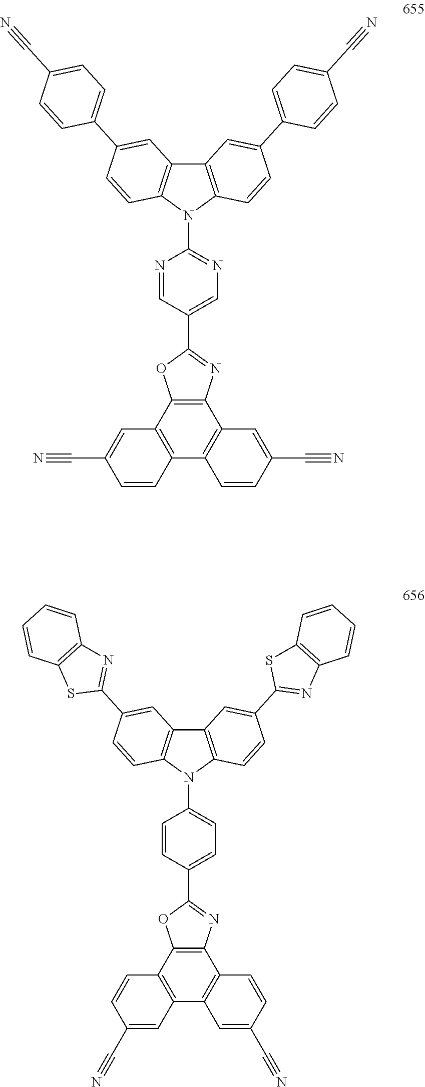 [SMILES: N#Cc1ccc(-c2ccc3c(c2)c2cc(-c4ccc(C#N)cc4)ccc2n3-c2ncc(-c3nc4c5cc(C#N)ccc5c5ccc(C#N)cc5c4o3)cn2)cc1 ; N#Cc1ccc2c(c1)c1cc(C#N)ccc1c1oc(-c3ccc(-n4c5ccc(-c6nc7ccccc7s6)cc5c5cc(-c6nc7ccccc7s6)ccc54)cc3)nc21]

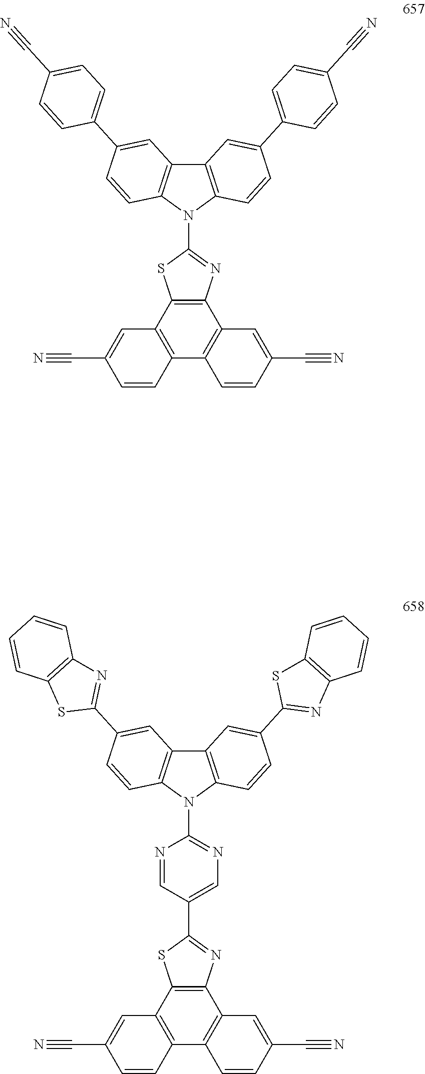 [SMILES: N#Cc1ccc(-c2ccc3c(c2)c2cc(-c4ccc(C#N)cc4)ccc2n3-c2nc3c4cc(C#N)ccc4c4ccc(C#N)cc4c3s2)cc1 ; N#Cc1ccc2c3ccc(C#N)cc3c3sc(-c4cnc(-n5c6ccc(-c7nc8ccccc8s7)cc6c6cc(-c7nc8ccccc8s7)ccc65)nc4)nc3c2c1]

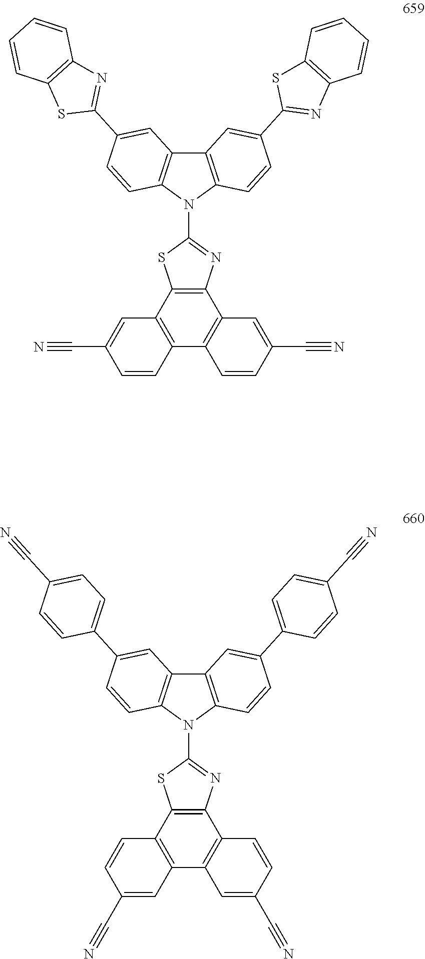 [SMILES: N#Cc1ccc2c3ccc(C#N)cc3c3sc(-n4c5ccc(-c6nc7ccccc7s6)cc5c5cc(-c6nc7ccccc7s6)ccc54)nc3c2c1 ; N#Cc1ccc(-c2ccc3c(c2)c2cc(-c4ccc(C#N)cc4)ccc2n3-c2nc3c4ccc(C#N)cc4c4cc(C#N)ccc4c3s2)cc1]

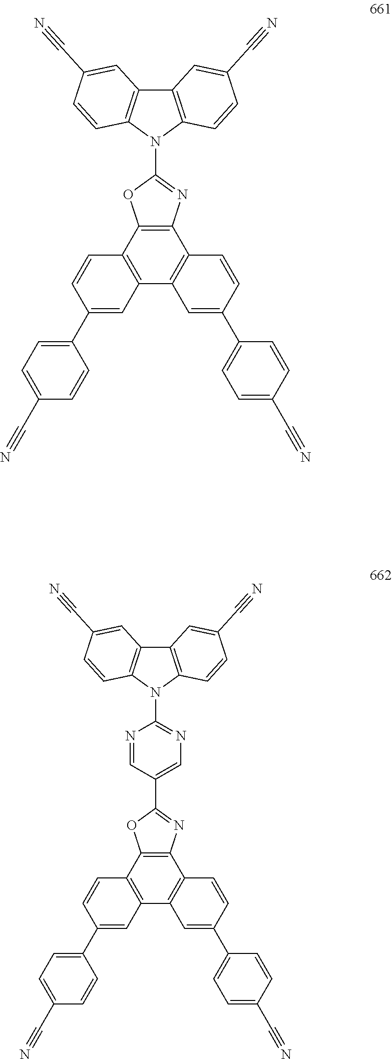 [SMILES: N#Cc1ccc(-c2ccc3c(c2)c2cc(-c4ccc(C#N)cc4)ccc2c2oc(-n4c5ccc(C#N)cc5c5cc(C#N)ccc54)nc32)cc1 ; N#Cc1ccc(-c2ccc3c(c2)c2cc(-c4ccc(C#N)cc4)ccc2c2oc(-c4cnc(-n5c6ccc(C#N)cc6c6cc(C#N)ccc65)nc4)nc32)cc1]

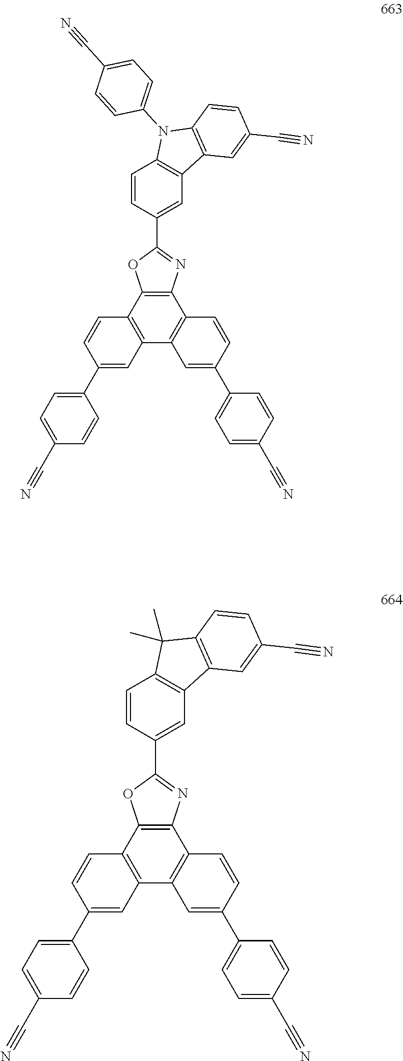 [SMILES: N#Cc1ccc(-c2ccc3c(c2)c2cc(-c4ccc(C#N)cc4)ccc2c2oc(-c4ccc5c(c4)c4cc(C#N)ccc4n5-c4ccc(C#N)cc4)nc32)cc1 ; CC1(C)c2ccc(C#N)cc2-c2cc(-c3nc4c5ccc(-c6ccc(C#N)cc6)cc5c5cc(-c6ccc(C#N)cc6)ccc5c4o3)ccc21]

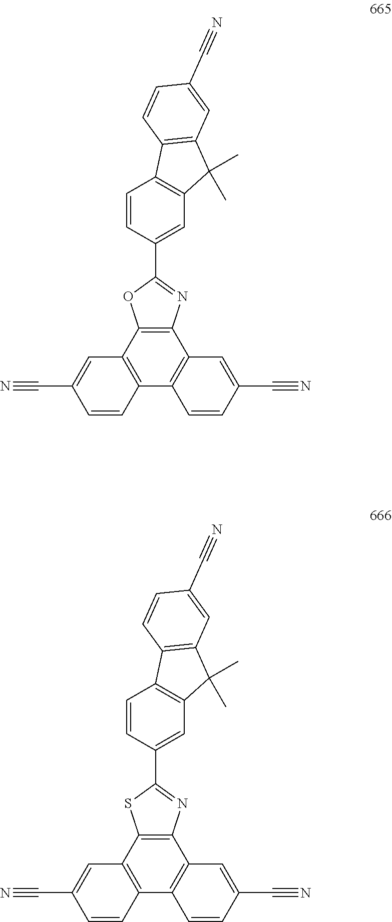 [SMILES: CC1(C)c2cc(C#N)ccc2-c2ccc(-c3nc4c5cc(C#N)ccc5c5ccc(C#N)cc5c4o3)cc21 ; CC1(C)c2cc(C#N)ccc2-c2ccc(-c3nc4c5cc(C#N)ccc5c5ccc(C#N)cc5c4s3)cc21]

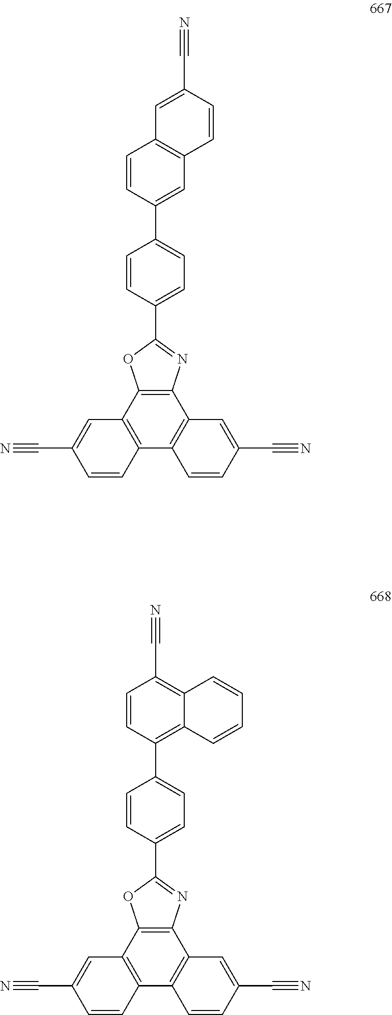 [SMILES: N#Cc1ccc2cc(-c3ccc(-c4nc5c6cc(C#N)ccc6c6ccc(C#N)cc6c5o4)cc3)ccc2c1 ; N#Cc1ccc2c3ccc(C#N)cc3c3oc(-c4ccc(-c5ccc(C#N)c6ccccc56)cc4)nc3c2c1]

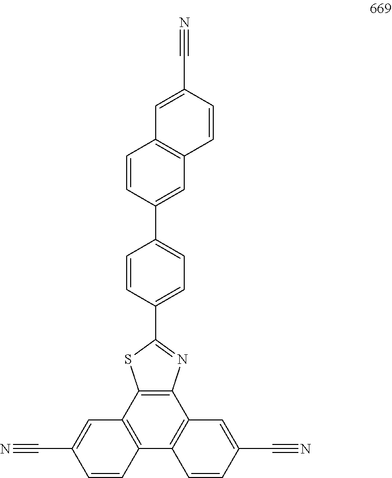 [SMILES: N#Cc1ccc2cc(-c3ccc(-c4nc5c6cc(C#N)ccc6c6ccc(C#N)cc6c5s4)cc3)ccc2c1]

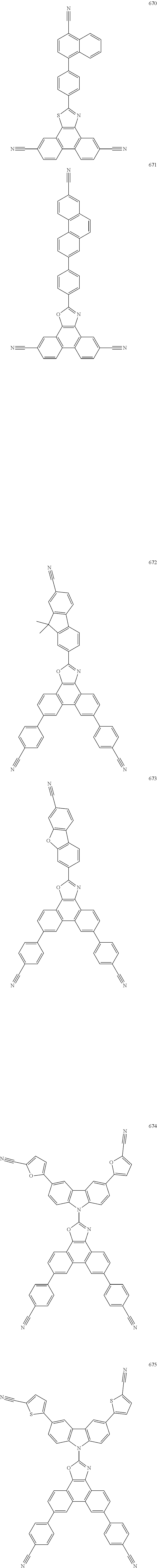

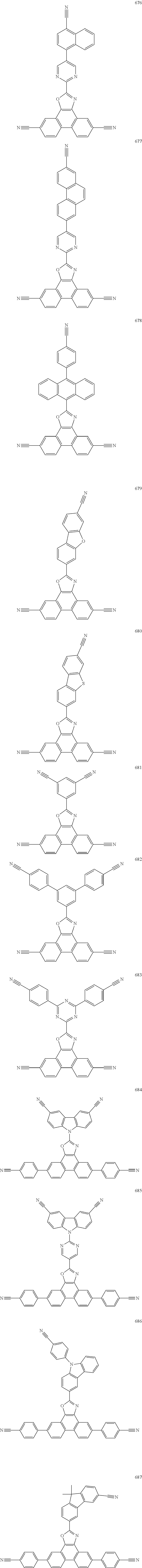

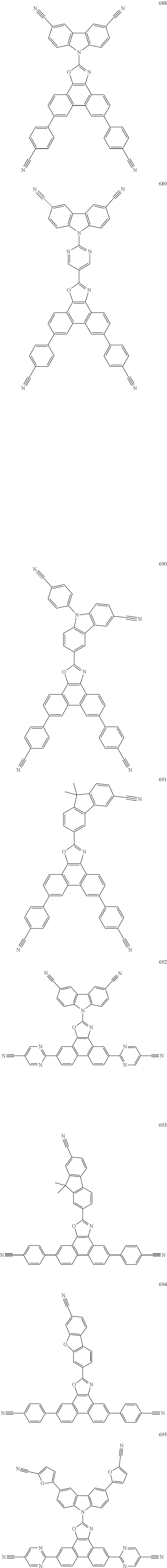

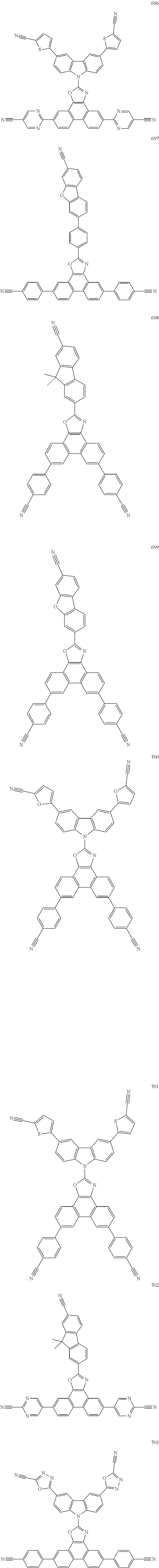

[chemical structure]

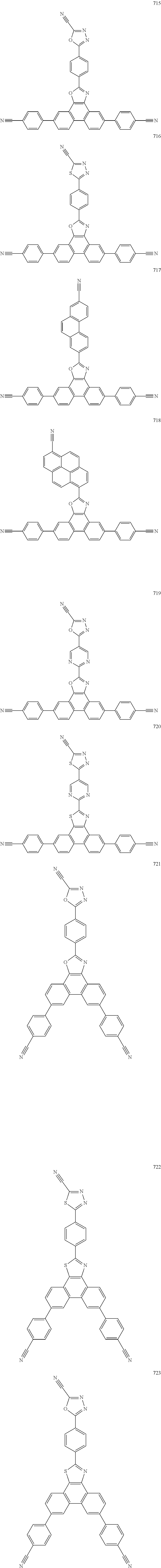

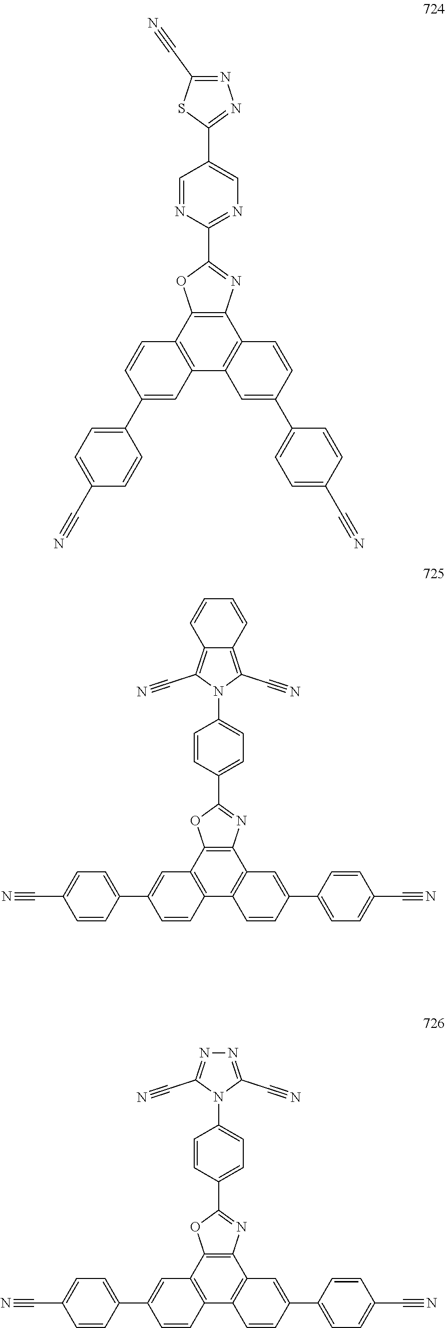 [SMILES: N#Cc1ccc(-c2ccc3c(c2)c2cc(-c4ccc(C#N)cc4)ccc2c2oc(-c4ncc(-c5nnc(C#N)s5)cn4)nc32)cc1 ; N#Cc1ccc(-c2ccc3c4ccc(-c5ccc(C#N)cc5)cc4c4oc(-c5ccc(-n6c(C#N)c7ccccc7c6C#N)cc5)nc4c3c2)cc1 ; N#Cc1ccc(-c2ccc3c4ccc(-c5ccc(C#N)cc5)cc4c4oc(-c5ccc(-n6c(C#N)nnc6C#N)cc5)nc4c3c2)cc1]

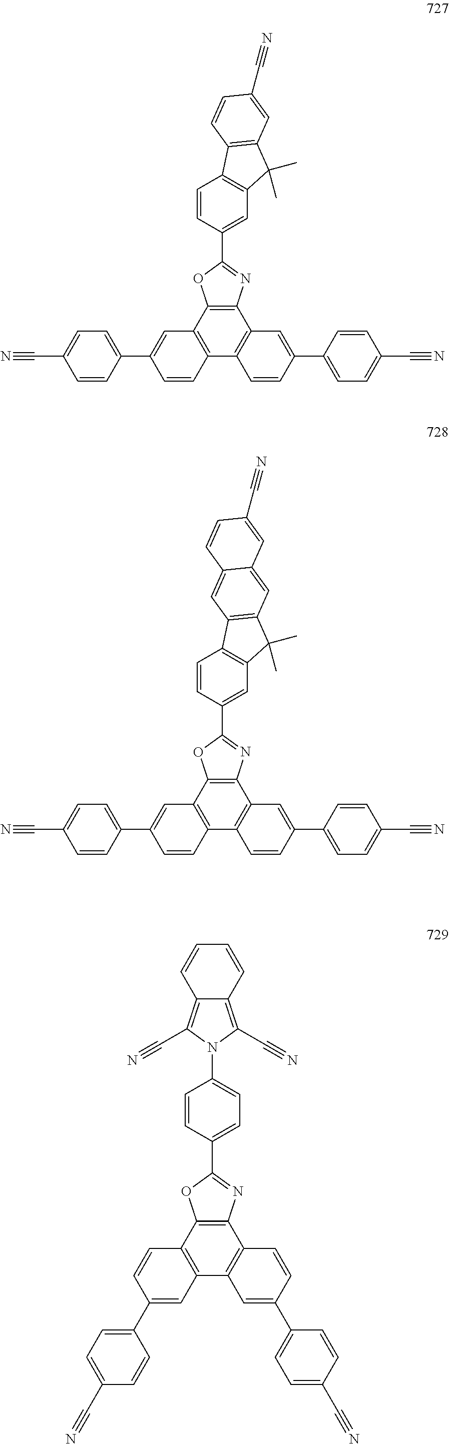 [SMILES: CC1(C)c2cc(C#N)ccc2-c2ccc(-c3nc4c5cc(-c6ccc(C#N)cc6)ccc5c5ccc(-c6ccc(C#N)cc6)cc5c4o3)cc21 ; CC1(C)c2cc(-c3nc4c5cc(-c6ccc(C#N)cc6)ccc5c5ccc(-c6ccc(C#N)cc6)cc5c4o3)ccc2-c2cc3ccc(C#N)cc3cc21 ; N#Cc1ccc(-c2ccc3c(c2)c2cc(-c4ccc(C#N)cc4)ccc2c2oc(-c4ccc(-n5c(C#N)c6ccccc6c5C#N)cc4)nc32)cc1]

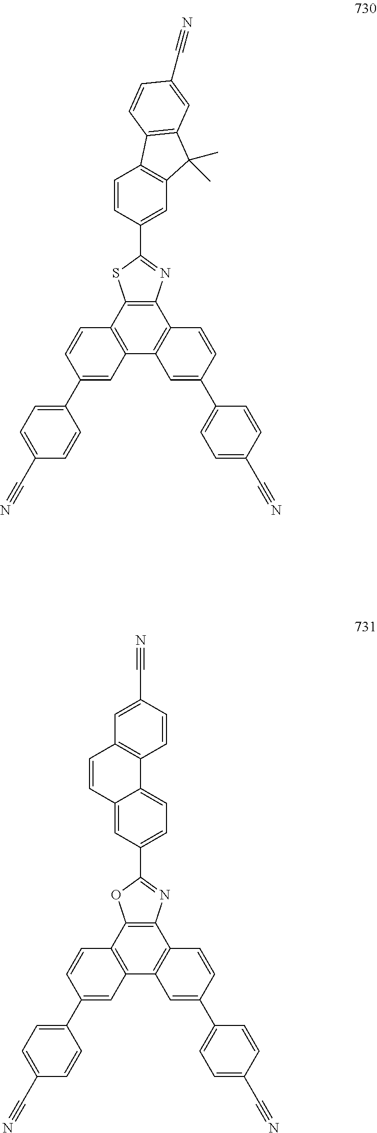 [SMILES: CC1(C)c2cc(C#N)ccc2-c2ccc(-c3nc4c5ccc(-c6ccc(C#N)cc6)cc5c5cc(-c6ccc(C#N)cc6)ccc5c4s3)cc21 ; N#Cc1ccc(-c2ccc3c(c2)c2cc(-c4ccc(C#N)cc4)ccc2c2oc(-c4ccc5c(ccc6cc(C#N)ccc65)c4)nc32)cc1]

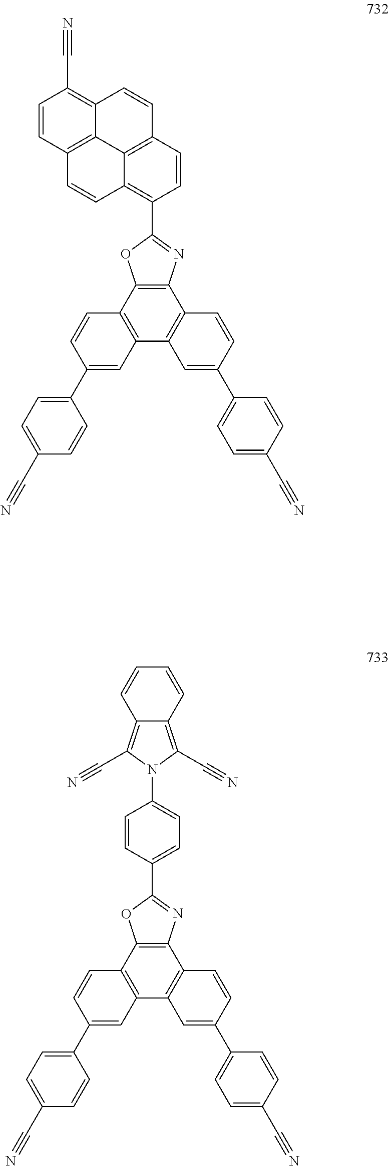 [SMILES: N#Cc1ccc(-c2ccc3c(c2)c2cc(-c4ccc(C#N)cc4)ccc2c2oc(-c4ccc5ccc6c(C#N)ccc7ccc4c5c76)nc32)cc1 ; N#Cc1ccc(-c2ccc3c(c2)c2cc(-c4ccc(C#N)cc4)ccc2c2oc(-c4ccc(-n5c(C#N)c6ccccc6c5C#N)cc4)nc32)cc1]

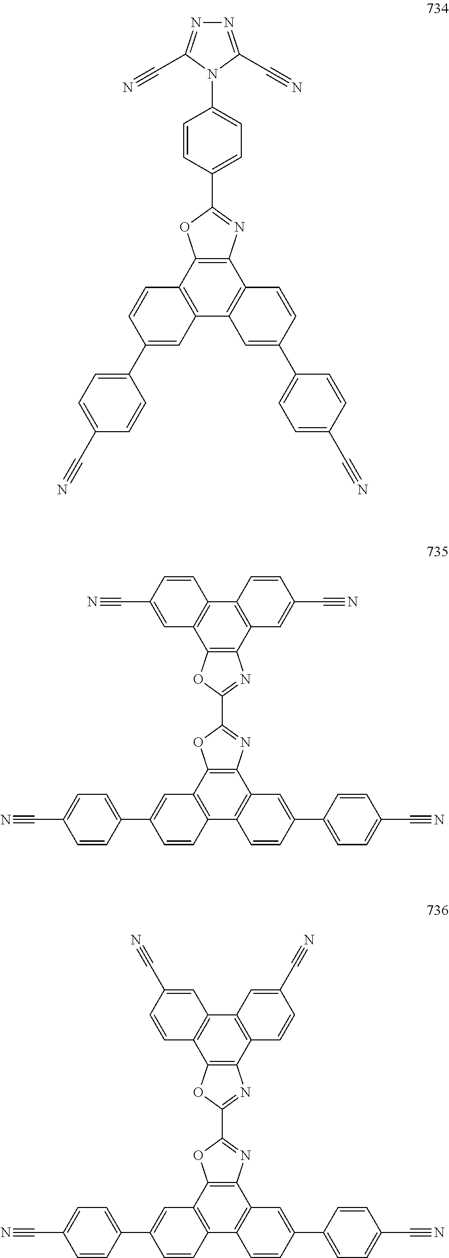 [SMILES: N#Cc1ccc(-c2ccc3c(c2)c2cc(-c4ccc(C#N)cc4)ccc2c2oc(-c4ccc(-n5c(C#N)nnc5C#N)cc4)nc32)cc1 ; N#Cc1ccc(-c2ccc3c4ccc(-c5ccc(C#N)cc5)cc4c4oc(-c5nc6c7cc(C#N)ccc7c7ccc(C#N)cc7c6o5)nc4c3c2)cc1 ; N#Cc1ccc(-c2ccc3c4ccc(-c5ccc(C#N)cc5)cc4c4oc(-c5nc6c7ccc(C#N)cc7c7cc(C#N)ccc7c6o5)nc4c3c2)cc1]

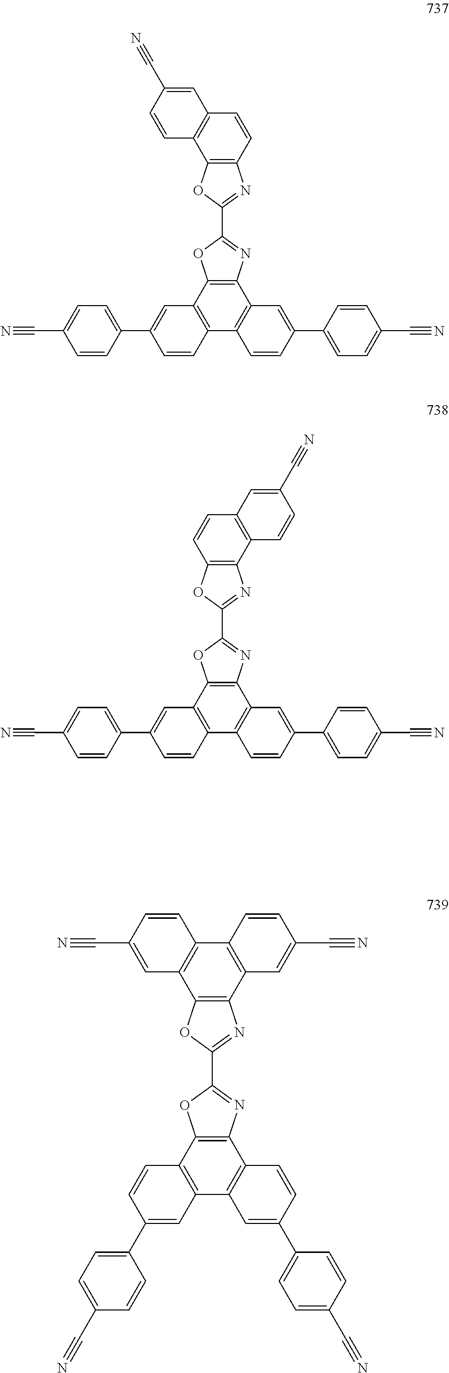 [SMILES: N#Cc1ccc(-c2ccc3c4ccc(-c5ccc(C#N)cc5)cc4c4oc(-c5nc6ccc7cc(C#N)ccc7c6o5)nc4c3c2)cc1 ; N#Cc1ccc(-c2ccc3c4ccc(-c5ccc(C#N)cc5)cc4c4oc(-c5nc6c(ccc7cc(C#N)ccc76)o5)nc4c3c2)cc1 ; N#Cc1ccc(-c2ccc3c(c2)c2cc(-c4ccc(C#N)cc4)ccc2c2oc(-c4nc5c6cc(C#N)ccc6c6ccc(C#N)cc6c5o4)nc32)cc1]

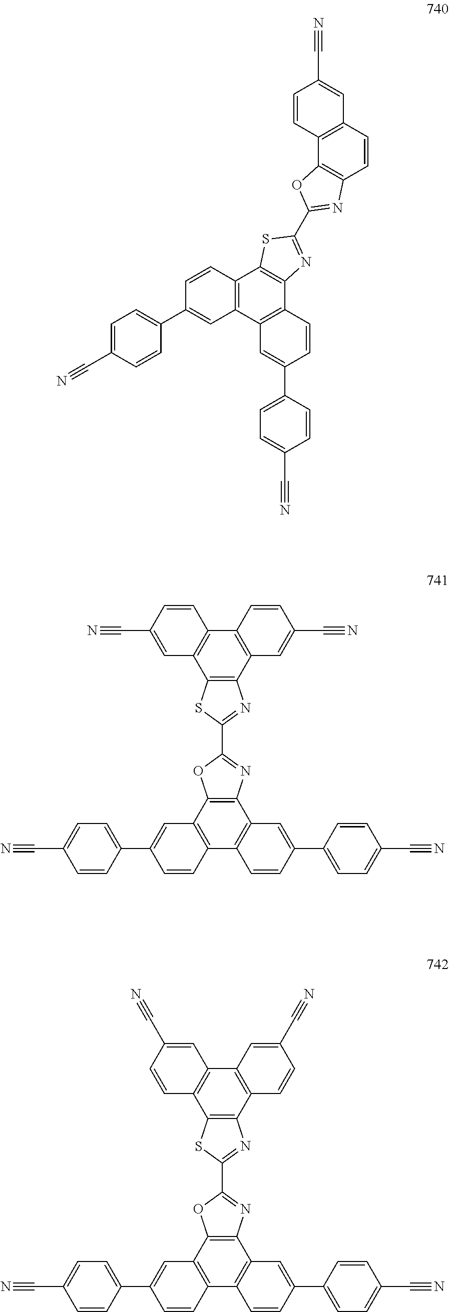 [SMILES: N#Cc1ccc(-c2ccc3c(c2)c2cc(-c4ccc(C#N)cc4)ccc2c2sc(-c4nc5ccc6cc(C#N)ccc6c5o4)nc32)cc1 ; N#Cc1ccc(-c2ccc3c4ccc(-c5ccc(C#N)cc5)cc4c4oc(-c5nc6c7cc(C#N)ccc7c7ccc(C#N)cc7c6s5)nc4c3c2)cc1 ; N#Cc1ccc(-c2ccc3c4ccc(-c5ccc(C#N)cc5)cc4c4oc(-c5nc6c7ccc(C#N)cc7c7cc(C#N)ccc7c6s5)nc4c3c2)cc1]

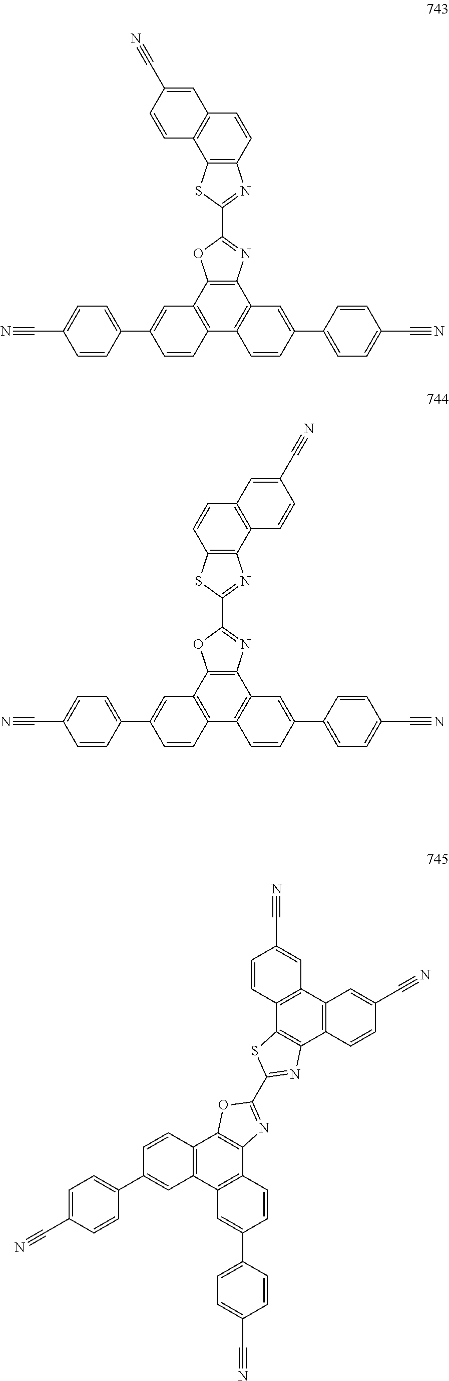 [SMILES: N#Cc1ccc(-c2ccc3c4ccc(-c5ccc(C#N)cc5)cc4c4oc(-c5nc6ccc7cc(C#N)ccc7c6s5)nc4c3c2)cc1 ; N#Cc1ccc(-c2ccc3c4ccc(-c5ccc(C#N)cc5)cc4c4oc(-c5nc6c(ccc7cc(C#N)ccc76)s5)nc4c3c2)cc1 ; N#Cc1ccc(-c2ccc3c(c2)c2cc(-c4ccc(C#N)cc4)ccc2c2oc(-c4nc5c6ccc(C#N)cc6c6cc(C#N)ccc6c5s4)nc32)cc1]

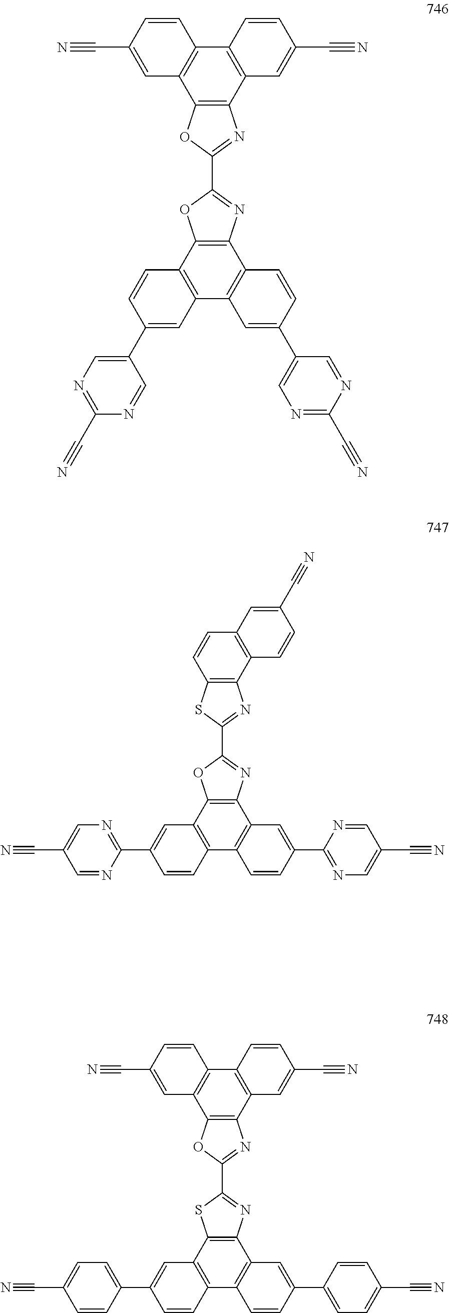 [SMILES: N#Cc1ccc2c3ccc(C#N)cc3c3oc(-c4nc5c6ccc(-c7cnc(C#N)nc7)cc6c6cc(-c7cnc(C#N)nc7)ccc6c5o4)nc3c2c1 ; N#Cc1cnc(-c2ccc3c4ccc(-c5ncc(C#N)cn5)cc4c4oc(-c5nc6c(ccc7cc(C#N)ccc76)s5)nc4c3c2)nc1 ; N#Cc1ccc(-c2ccc3c4ccc(-c5ccc(C#N)cc5)cc4c4sc(-c5nc6c7cc(C#N)ccc7c7ccc(C#N)cc7c6o5)nc4c3c2)cc1]

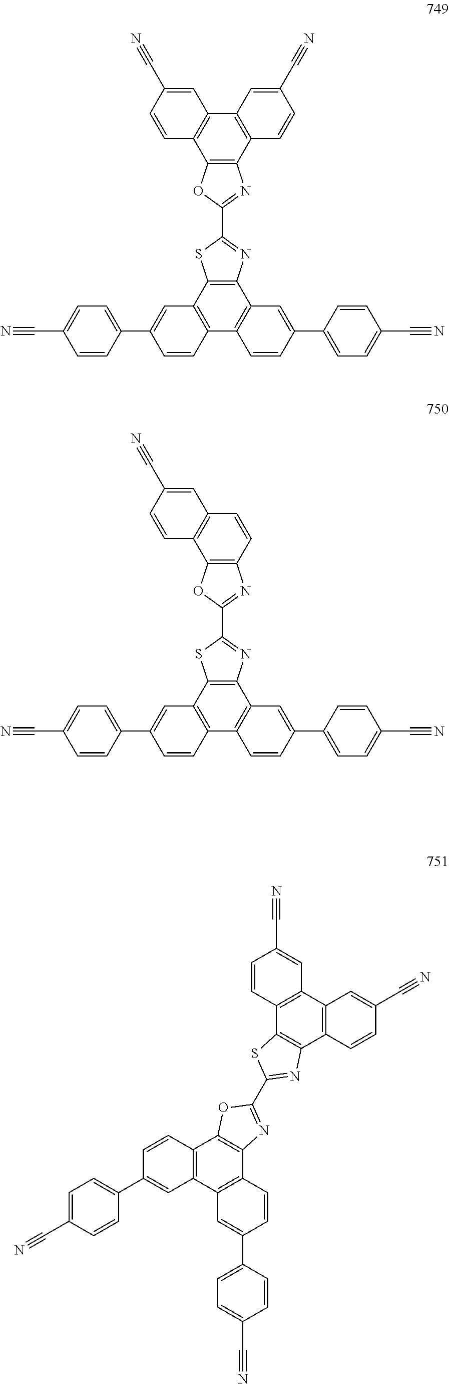 [SMILES: N#Cc1ccc(-c2ccc3c4ccc(-c5ccc(C#N)cc5)cc4c4sc(-c5nc6c7ccc(C#N)cc7c7cc(C#N)ccc7c6o5)nc4c3c2)cc1 ; N#Cc1ccc(-c2ccc3c4ccc(-c5ccc(C#N)cc5)cc4c4sc(-c5nc6ccc7cc(C#N)ccc7c6o5)nc4c3c2)cc1 ; N#Cc1ccc(-c2ccc3c(c2)c2cc(-c4ccc(C#N)cc4)ccc2c2oc(-c4nc5c6ccc(C#N)cc6c6cc(C#N)ccc6c5s4)nc32)cc1]

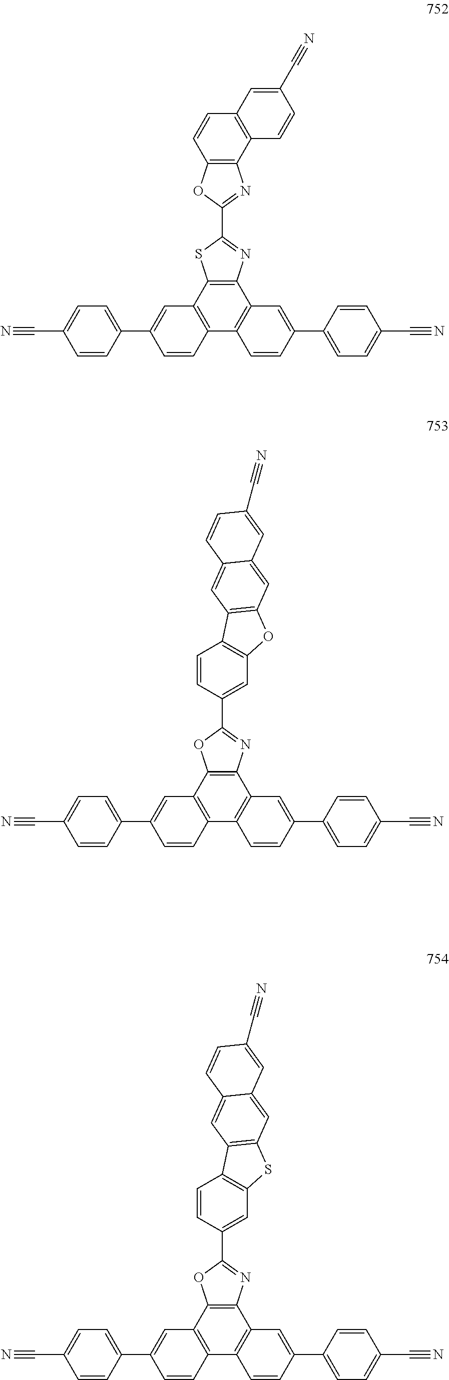 [SMILES: N#Cc1ccc(-c2ccc3c4ccc(-c5ccc(C#N)cc5)cc4c4sc(-c5nc6c(ccc7cc(C#N)ccc76)o5)nc4c3c2)cc1 ; N#Cc1ccc(-c2ccc3c4ccc(-c5ccc(C#N)cc5)cc4c4oc(-c5ccc6c(c5)oc5cc7cc(C#N)ccc7cc56)nc4c3c2)cc1 ; N#Cc1ccc(-c2ccc3c4ccc(-c5ccc(C#N)cc5)cc4c4oc(-c5ccc6c(c5)sc5cc7cc(C#N)ccc7cc56)nc4c3c2)cc1]

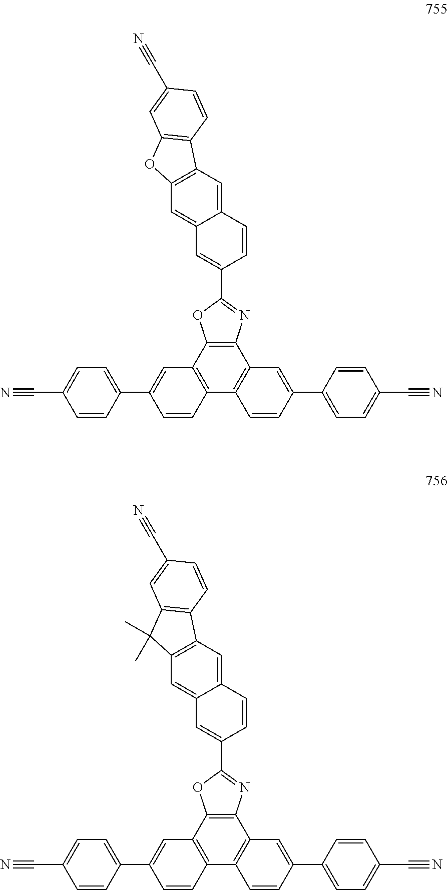 [SMILES: N#Cc1ccc(-c2ccc3c4ccc(-c5ccc(C#N)cc5)cc4c4oc(-c5ccc6cc7c(cc6c5)oc5cc(C#N)ccc57)nc4c3c2)cc1 ; CC1(C)c2cc(C#N)ccc2-c2cc3ccc(-c4nc5c6cc(-c7ccc(C#N)cc7)ccc6c6ccc(-c7ccc(C#N)cc7)cc6c5o4)cc3cc21]

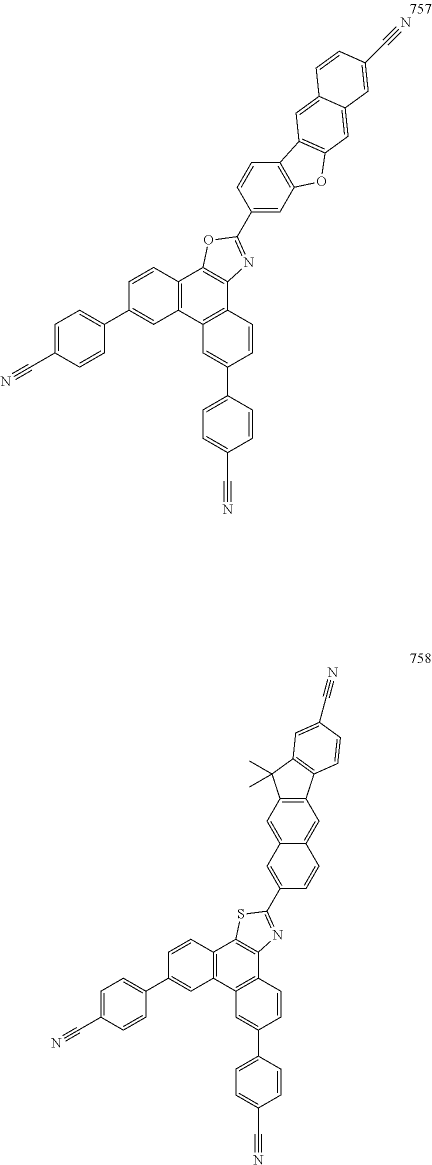 [SMILES: N#Cc1ccc(-c2ccc3c(c2)c2cc(-c4ccc(C#N)cc4)ccc2c2oc(-c4ccc5c(c4)oc4cc6cc(C#N)ccc6cc45)nc32)cc1 ; CC1(C)c2cc(C#N)ccc2-c2cc3ccc(-c4nc5c6ccc(-c7ccc(C#N)cc7)cc6c6cc(-c7ccc(C#N)cc7)ccc6c5s4)cc3cc21]

As described above, the organic compound according to the present invention may be implemented by synthesizing organic compounds having various properties using moieties having characteristic skeleton structures and inherent properties, and as a result, when the organic compound according to the present invention is applied to a light efficiency improving layer provided in an organic light-emitting device, it is possible to further improve not only the low voltage driving characteristics of the device, but also the light-emitting efficiency, color purity, service life characteristics, and the like.

In addition, the compound of an embodiment of the present invention may be applied to a device according to a general method for manufacturing an organic light emitting device.

The organic light emitting device according to an exemplary embodiment of the present invention may be composed of a structure including a first electrode, a second electrode and an organic layer disposed therebetween, and may be manufactured using typical device manufacturing methods and materials, except that the organic light emitting compound according to the present invention is used in an organic layer of the device.

The organic layer of the organic light emitting device according to the present invention may be composed of a single-layered structure, but may also be composed of a multi-layered structure in which two or more organic layers are stacked. For example, the organic layer may have a structure including a hole injection layer, a hole transport layer, a light-emitting layer, an electron transport layer, an electron injection layer, an electron blocking layer, a hole blocking layer, a light efficiency improving layer (capping layer), etc. However, the structure of the organic layer is not limited thereto, and may include a fewer or greater number of organic layers.

In addition, the organic electroluminescent device may include a substrate, a first electrode (anode), an organic layer, a second electrode (cathode), and a light efficiency improving layer (capping layer), of which may be formed under the first electrode (bottom emission type) or on the second electrode (top emission type).

When the organic electroluminescent device is of a top emission type, light from the light emitting layer is emitted to the cathode and passes through the light efficiency improving layer (CPL) formed using the compound according to an embodiment of the present invention having a relatively high refractive index. The wavelength of the light is amplified, resulting in an increase in luminous efficiency.

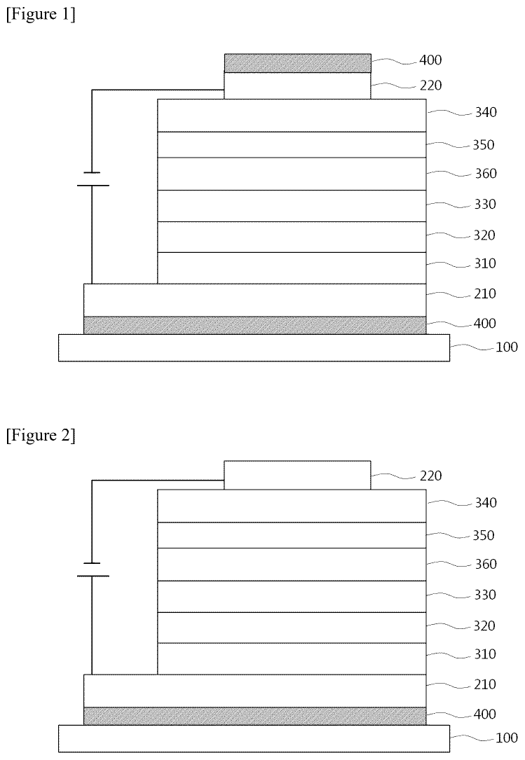

A specific embodiment of the organic light-emitting device according to the present invention will be described with reference to the following FIGS. 1 to 3.

The organic light-emitting device may include a substrate 100, a first electrode (210), a second electrode (220), one or more organic layers (310 to 360) interposed on the inner side of the first electrode and the second electrode, and a light efficiency improving layer (400), and the light efficiency improving layer may be disposed on the outer side of at least one of the first electrode and the second electrode.

Of both sides of the first electrode or the second electrode, the side adjacent to the organic layer interposed between the first electrode and the second electrode is referred to as the inner side, and the side not adjacent to the organic layer is referred to as the outer side. That is, accordingly, in the organic light-emitting device according to the present invention, when a light efficiency improving layer (400) is disposed on the outer side of the first electrode (210), the first electrode (210) is interposed between the light efficiency improving layer (400) and the organic layers (310 to 360), and when the light efficiency improving layer 400 is disposed on the outer side of the second electrode (220), the second electrode (220) is interposed between the light efficiency improving layer (400) and the organic layers (310 to 360).

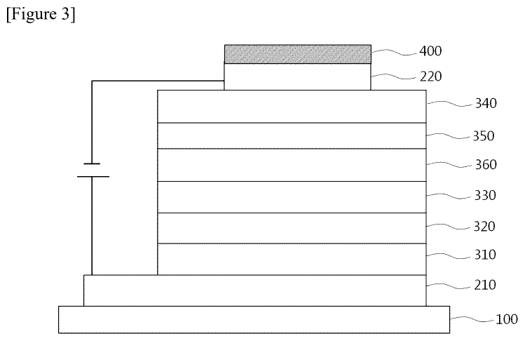

In this case, the method of forming a light efficiency improving layer on the second electrode as shown in the following FIG. 3 (top emission) may further include a reflection layer (not shown) to reflect light emitted between the first electrode (210) and the substrate (100), thereby further emitting the light toward the top of the second electrode.

As described above, for the organic light-emitting device according to the present invention, various organic layers having one or more layers may be interposed on the inner sides of the first electrode and the second electrode, and a light efficiency improving layer may be formed on the outer side of at least one of the first electrode and the second electrode. That is, the light efficiency improving layer may be formed on both the outer sides of the first electrode and the second electrode, or may be formed only on the outer side of the first electrode or only on the outer side of the second electrode.

In this case, the light efficiency improving layer may include a compound for a light efficiency improving layer according to the present invention, and may include a compound for a light efficiency improving layer according to the present invention alone or two or more of the compound or in combination with a known compound, and the light efficiency improving layer may have a thickness of 100 Å to 4,000 Å.

In addition, in the organic light-emitting device according to the present invention, the light efficiency improving layer may be configured as a composite light efficiency improving layer structure in which a first light efficiency improving layer having a relatively low refractive index and a second light efficiency improving layer having a higher refractive index than the first light efficiency improving layer are stacked, and the stacking order of the first light efficiency improving layer and the second light efficiency improving layer due to the difference in refractive index is not limited. The first light efficiency improving layer may be disposed on the outer side of the second light efficiency improving layer, and conversely, the second light efficiency improving layer may be disposed on the outer side of the first light efficiency improving layer.

Therefore, according to an embodiment of the present invention, the low refractive index compound represented by [Formula I] above according to the present invention may be applied to the first light efficiency improving layer.

Furthermore, each of the first light efficiency improving layer and the second light efficiency improving layer has a multi-layered structure in which a plurality of first light efficiency improving layers and a plurality of second light efficiency improving layers are stacked, and in this case, the first light efficiency improving layers and the second light efficiency improving layers may be stacked alternately, and the stacking order is also not limited.

Meanwhile, in the organic light-emitting device according to the present invention, the light efficiency improving layer may have a structure having a refractive index gradient, and for the refractive index gradient, the refractive index may gradually decrease toward the outside, or the refractive index may gradually increase toward the outside. For this purpose, the light efficiency improving layer may be deposited by gradually changing the concentration of the compound for the light efficiency improving layer according to the present invention, thereby implementing a gradient in the refractive index in the light efficiency improving layer.

Preferred structures of the organic layers of the organic light emitting according to an embodiment of the present invention will be explained in more detail in the examples to be described later.

Further, the organic light emitting device according to the present invention may be manufactured by depositing a metal or a metal oxide having conductivity, or an alloy thereof on a substrate to form a positive electrode, forming an organic layer including a hole injection layer, a hole transport layer, a light emitting layer, and an electron transport layer thereon, and then depositing a material, which may be used as a negative electrode, thereon, by using a physical vapor deposition (PVD) method such as sputtering or e-beam evaporation.

In addition, the organic electroluminescent device of an embodiment of the present invention may be manufactured by depositing a metal, a conductive metal oxide or an alloy thereof on a substrate by a physical vapor deposition (PVD) method such as sputtering or e-beam evaporation to form an anode, forming organic layers including a hole injecting layer, a hole transport layer, a light emitting layer, and an electron transport layer thereon, and depositing a cathode material thereon.

In addition to the above methods, the organic light emitting device may be fabricated by depositing a anode (210) material, organic layer materials (310˜360), and an cathode (220) material in this order on a substrate (100). The organic layers may have a multilayer structure including a hole injecting layer (310), a hole transport layer (320), an electron blocking layer (330), a light emitting layer (360), an electron transport layer (350) and an electron injecting layer (340), but is not limited thereto and may have a monolayer structure. In addition, the organic layers may be manufactured in a smaller number of layers by a solvent process using various polymer materials rather than by a deposition process, such as spin coating, dip coating, doctor blading, screen printing, inkjet printing or thermal transfer.

As the substrate (100), a substrate commonly used in organic light-emitting devices may be used, and the substrate (100) may be particularly a transparent glass substrate or a flexible plastic substrate having excellent mechanical strength, thermal stability, transparency, surface smoothness, ease of handling, and water resistance.

As the anode (210) material, a material having a high work function is generally preferred for easy injection of holes into the organic layers. Specific examples of anode materials suitable for use in an embodiment of the present invention include, but are not limited to: metals such as vanadium, chromium, copper, zinc, and gold and alloys thereof; metal oxides such as zinc oxide, indium oxide, indium thin oxide (ITO), and indium zinc oxide (IZO); combinations of metals and oxides such as ZnO:Al and SnO₂:Sb; and conductive polymers such as poly(3-methylthiophene), poly[3,4-(ethylene-1,2-dioxy)thiophene](PEDT), polypyrrole, and polyaniline.

As the cathode (220) material, a material having a low work function is generally preferred for easy injection of electrons into the organic layers. Specific examples of suitable cathode materials include, but are not limited to: metals such as magnesium, calcium, sodium, potassium, titanium, indium, yttrium, lithium, gadolinium, aluminum, silver, tin, and lead and alloys thereof; and multilayer structure materials such as LiF/Al and LiO₂/Al.

The hole injecting layer (310) material is preferably a material that may receive holes injected from the anode at low voltage. The highest occupied molecular orbital (HOMO) of the hole injecting material is preferably between the work function of the anode material and the HOMO of the adjacent organic layer. Specific examples of hole injecting materials include, but are not limited to, metal porphyrin, oligothiophene, arylamine-based organic materials, hexanitrile hexaazatriphenylene, quinacridone-based organic materials, perylene-based organic materials, anthraquinone, polyaniline, and polythiophene-based conductive polymers.

The hole transport layer (320) material is a material that may receive holes transported from the anode or the hole injecting layer and may transfer the holes to the light emitting layer. A material with high hole mobility is suitable. Specific examples thereof include arylamine-based organic materials, conductive polymers, and block copolymers consisting of conjugated and non-conjugated segments. The use of the organic compound according to an embodiment of the present invention ensures further improved low-voltage driving characteristics, high luminous efficiency, and life characteristics of the device.

The light emitting layer (360) material is a material that may receive and recombine holes from the hole transport layer and electrons from the electron transport layer to emit light in the visible ray area. A material with high quantum efficiency for fluorescence and phosphorescence is preferred. Specific examples thereof include, but are not limited to, 8-hydroxyquinoline aluminum complex (Alq₃), carbazole-based compounds, dimerized styryl compounds, BAlq, 10-hydroxybenzoquinoline-metal compounds, benzoxazole-based compounds, benzthiazole-based compounds, and benzimidazole-based compounds, poly(p-phenylenevinylene) (PPV)-based polymers, spiro compounds, polyfluorene, and rubrene.

The electron transport layer (350) material is a material that may receive electrons injected from the cathode and may transfer the electrons to the light emitting layer. A material with high electron mobility is suitable. Specific examples thereof include, but are not limited to, 8-hydroxyquinoline Al complex, Alq₃ complexes, organic radical compounds, hydroxyflavone-metal complexes.

The electron injection layer (340) material may be formed by depositing an electron injection layer material on the electron transport layer, and a known material such as LiF, NaCl, CsF, L₁₂O, and BaO may be used as the electron injection layer material.

Furthermore, although not shown in FIGS. 1 to 3, according to an embodiment of the present invention, organic layers having various functions may be additionally formed between the light efficiency improving layer (400) and the first electrode (210) or between the light efficiency improving layer (400) and the second electrode (220), and organic layers having various functions may also be additionally formed on the upper and lower parts (outer surfaces) of the capping layer (400).

The organic light emitting device according to the present disclosure may be a top-emission type, a bottom-emission type or a dual-emission type depending on the materials used.

The organic light emitting device according to an embodiment of the present invention may be of a top emission, bottom emission or dual emission type according to the materials used.

Furthermore, the organic light emitting compound according to the present invention may be operated by a principle which is similar to the principle applied to an organic light emitting device, even in an organic electroluminescent device including an organic solar cell, an organic photoconductor, an organic transistor, and the like.

In addition, the organic compound according to an embodiment of the present invention may perform its function even in organic electronic devices, including organic solar cells, organic photoconductors, and organic transistors, based on a similar principle to that applied to the organic light emitting device.

Hereinafter, the present invention will be exemplified in more detail through preferred examples. However, these examples are for more specifically describing the present invention, the scope of the present invention is not limited thereto, and it will be obvious to a person with ordinary skill in the art that various changes and modifications can be made within the scope of the present invention and the scope of the technical spirit.

### Synthesis Example 1: Synthesis of Compound 70

### (1) Preparation Example 1: Synthesis of Intermediate 70-1

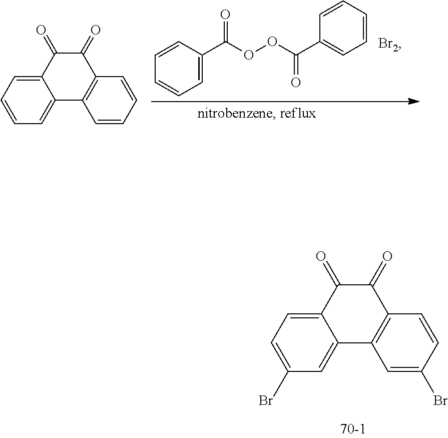

After 100 g of phenanthrene-9,10-dione, 2.9 g of dibenzoyl peroxide, and 10 g of Br₂ were added to 100 mL of nitrobenzene, the resulting mixture was stirred and warmed to 120° C., and then when HBr began to be produced, 176 g of Br₂ was dropped and then the resulting mixture was stirred for 2 hours. After the completion of the reaction was confirmed by TLC and HPLC, the resulting product was cooled to room temperature and solidified by adding EtOH. Thereafter, the solidified product was filtered and thoroughly washed with EtOH to obtain 25 g of yellow Intermediate 70-1.

### (2) Preparation Example 2: Synthesis of Intermediate 70-2

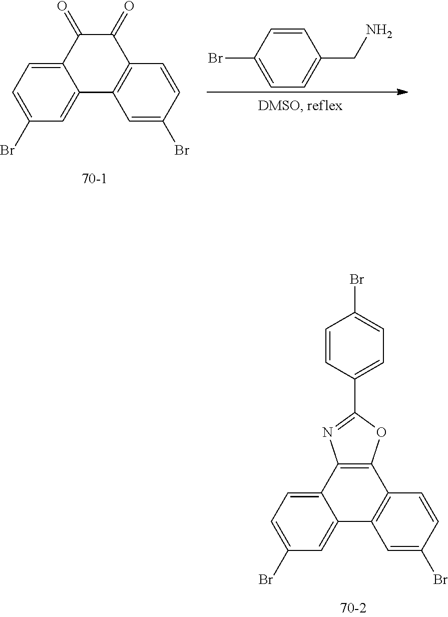 [SMILES: O=C1C(=O)c2ccc(Br)cc2-c2cc(Br)ccc21 ; NCc1ccc(Br)cc1 ; Brc1ccc(-c2nc3c4ccc(Br)cc4c4cc(Br)ccc4c3o2)cc1]

After 10 g of Intermediate 70-2 and 5.1 g of (4-bromophenyl)methanamine were added to 300 mL of DMSO, the resulting mixture was warmed to 200° C., and stirred for 24 hours. After the reaction was completed by TLC and HPLC, the resulting product was cooled to room temperature and solidified by adding MeOH. Thereafter, the solidified product was filtered and thoroughly washed with MeOH to obtain 7 g of yellow Intermediate 70-2.

### (3) Preparation Example 3: Synthesis of Compound 70

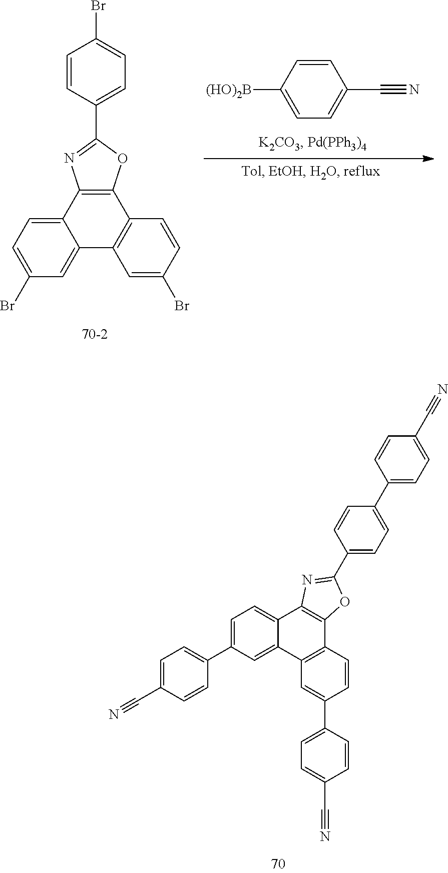 [SMILES: Brc1ccc(-c2nc3c4ccc(Br)cc4c4cc(Br)ccc4c3o2)cc1 ; N#Cc1ccc(B(O)O)cc1 ; N#Cc1ccc(-c2ccc(-c3nc4c5ccc(-c6ccc(C#N)cc6)cc5c5cc(-c6ccc(C#N)cc6)ccc5c4o3)cc2)cc1]

After 7 g of Intermediate 70-2, 7 g of (4-cyanophenyl)boronic acid, and 1.36 g of Pd cat. were added to toluene, 17 g of potassium carbonate (an aqueous solution) and ethanol were added, and the resulting mixture was warmed to 80° C. and stirred for 12 hours. After the reaction was completed by TLC and HPLC, the resulting product was cooled to room temperature, filtered, and then recrystallized with monochlorobenzene to obtain 3 g of Compound 70 (a pale yellow solid).

LC/MS: m/z=599[(M)+]

### Synthesis Example 2: Synthesis of Compound 132

### (1) Preparation Example 1: Synthesis of Intermediate 132-1

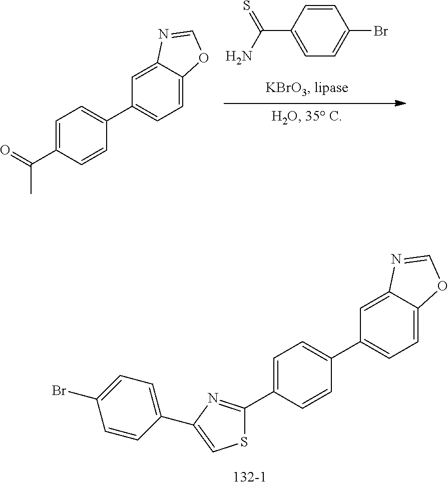

After 10 g of 1-(4-(benzo[d]oxazol-5-yl)phenyl)ethan-1-one, 6.07 g of 4-bromobenzothioamide, 21.12 g of KBrO₃, and 10 mol % of lipase were added to H₂O at 35° C., the resulting mixture was stirred for 10 minutes. After the completion of the reaction was confirmed by TLC and HPLC, extraction was performed with MC and H₂O to concentrate an organic layer. Thereafter, the solid was solidified with EtOH to obtain 11.8 g (yield 64.6%) of Intermediate 132-1.

### (2) Preparation Example 2. Synthesis of Intermediate 132-2

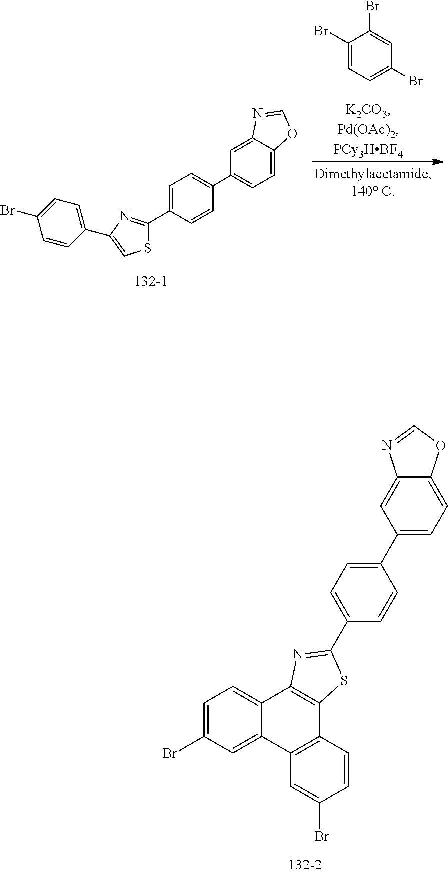 [SMILES: Brc1ccc(Br)c(Br)c1 ; Brc1ccc(-c2csc(-c3ccc(-c4ccc5ocnc5c4)cc3)n2)cc1 ; Brc1ccc2c(c1)c1cc(Br)ccc1c1sc(-c3ccc(-c4ccc5ocnc5c4)cc3)nc21]

After 10 g of Intermediate 132-1, 7.26 g of 1,2,4-tribromobenzene, 12.76 g of K₂CO₃, 0.26 g of Pd cat., and Pcy₃H·BF₄ were added to dimethylacetamide, the resulting mixture was stirred. After the mixture was warmed to 140° C. and heated for 14 hours, the completion of the reaction was confirmed by TLC and HPLC, and then the resulting product was cooled and filtered. Thereafter, the filtered product was purified using a silica gel column and then recrystallized to obtain 2.8 g (yield 20.7%) of Intermediate 132-2.

### (3) Preparation Example 3: Synthesis of Compound 132

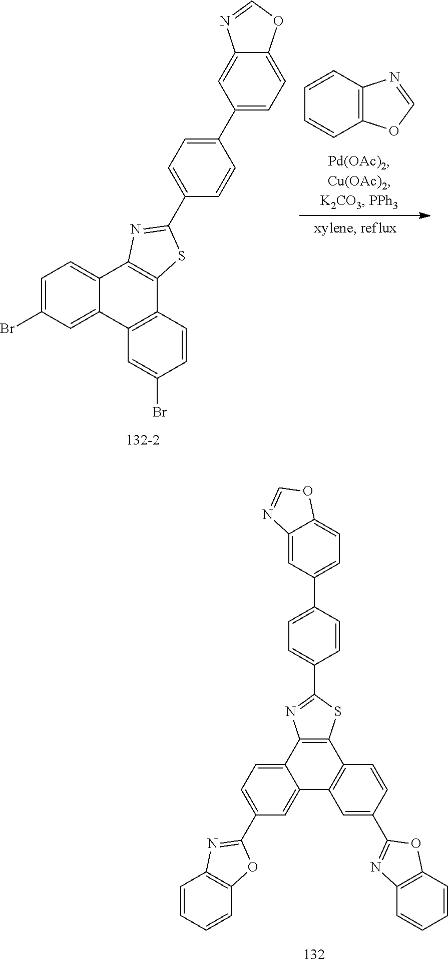

After 10 g of Intermediate 132-2, 8.87 g of benzo[d]oxazole, 10.3 g of K₂CO₃, 0.08 g of Pd cat., 1.35 g of Cu(OAc)₂, and 19.54 g of PPh₃ were added to xylene, the resulting mixture was stirred. After the mixture was warmed to 160° C. and heated for 12 hours, the completion of the reaction was confirmed by TLC and HPLC, and then the resulting product was cooled and filtered. Thereafter, the filtered product was purified using a silica gel column and then recrystallized to obtain 5.56 g (yield 45%) of Compound 132.

LC/MS: m/z=663[(M)+]

### Synthesis Example 3: Synthesis of Compound 145

### (1) Preparation Example 1: Synthesis of Intermediate 145-1

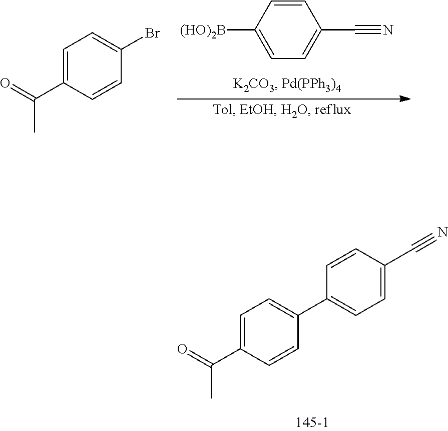

After 20 g of 1-(4-bromophenyl)ethan-1-one, 17.7 g of (4-cyanophenyl)boronic acid, 42 g of potassium carbonate (aq), and 3.5 g of Pd cat. were added to toluene and EtOH, the resulting mixture was stirred. Thereafter, after the mixture was warmed to 80° C. and reflux was maintained for 12 hours, the completion of the reaction was confirmed by TLC and HPLC, and then extraction was performed using MC and H₂O. Thereafter, an organic layer was separated and concentrated, and the resulting solid was purified using a column to obtain 17 g (yield 77%) of Intermediate 145-1.

### (2) Preparation Example 2: Synthesis of Intermediate 145-2

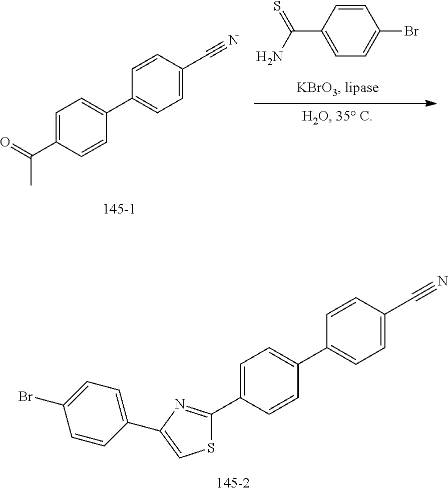 [SMILES: CC(=O)c1ccc(-c2ccc(C#N)cc2)cc1 ; NC(=S)c1ccc(Br)cc1 ; N#Cc1ccc(-c2ccc(-c3nc(-c4ccc(Br)cc4)cs3)cc2)cc1]

After 17 g of Intermediate 145-1 11.1 g of 4-bromobenzothioamide, 38.5 g of KBrO₃, and 10 mol % of lipase were added to H₂O at 35° C., the resulting mixture was stirred for 10 minutes. After the completion of the reaction was confirmed by TLC and HPLC, extraction was performed with MC and H₂O to concentrate an organic layer. Thereafter, the solid was solidified with EtOH to obtain 20.5 g (yield 64%) of Intermediate 145-2.

### (3) Preparation Example 3: Synthesis of Intermediate 145-3

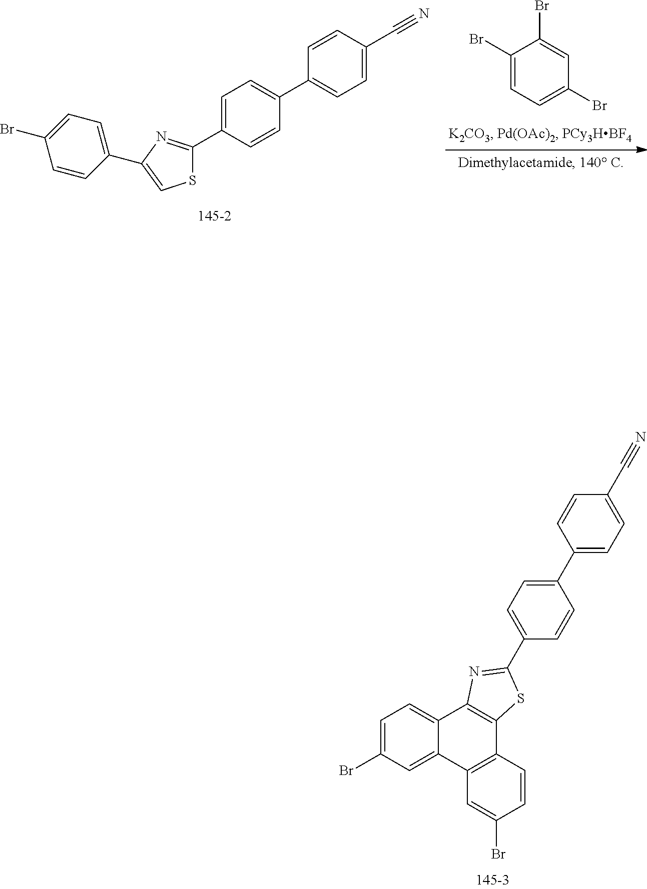 [SMILES: N#Cc1ccc(-c2ccc(-c3nc(-c4ccc(Br)cc4)cs3)cc2)cc1 ; Brc1ccc(Br)c(Br)c1 ; N#Cc1ccc(-c2ccc(-c3nc4c5ccc(Br)cc5c5cc(Br)ccc5c4s3)cc2)cc1]

After 20.5 g of Intermediate 145-2 10.3 g of 1,2,4-tribromobenzene, 18.4 g of K₂CO₃, 0.4 g of Pd cat., and Pcy₃H BF₄ were added to dimethylacetamide, the resulting mixture was stirred. After the mixture was warmed to 140° C. and heated for 14 hours, the completion of the reaction was confirmed by TLC and HPLC, and then the resulting product was cooled and filtered. Thereafter, the filtered product was purified using a silica gel column and then recrystallized to obtain 5 g (yield 18%) of Intermediate 145-3.

### (4) Preparation Example 4: Synthesis of Compound 145

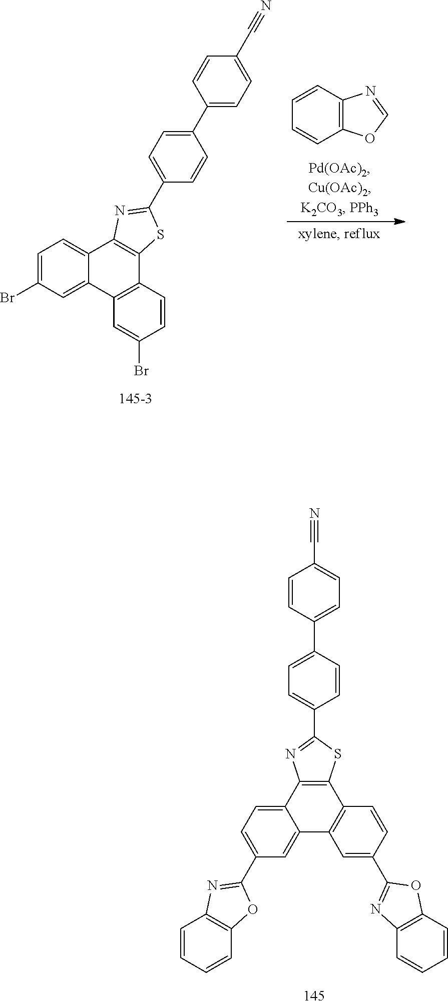 [SMILES: N#Cc1ccc(-c2ccc(-c3nc4c5ccc(Br)cc5c5cc(Br)ccc5c4s3)cc2)cc1 ; c1ccc2ocnc2c1 ; N#Cc1ccc(-c2ccc(-c3nc4c5ccc(-c6nc7ccccc7o6)cc5c5cc(-c6nc7ccccc7o6)ccc5c4s3)cc2)cc1]

After 5 g of Intermediate 145-3, 4.2 g of benzo[d]oxazole, 19.4 g of K₂CO₃, 0.04 g of Pd cat., 0.6 g of Cu(OAc)₂, and 2.3 g of PPh₃ were added to xylene, the resulting mixture was stirred. After the mixture was warmed to 160° C. and heated for 12 hours, the completion of the reaction was confirmed by TLC and HPLC, and then the resulting product was cooled and filtered. Thereafter, the filtered product was purified using a silica gel column and then recrystallized to obtain 2.8 g (yield 49%) of Compound 145.

LC/MS: m/z=647[(M)+]

### Synthesis Example 4: Synthesis of Compound 147

### (1) Preparation Example 1: Synthesis of Intermediate 147-1

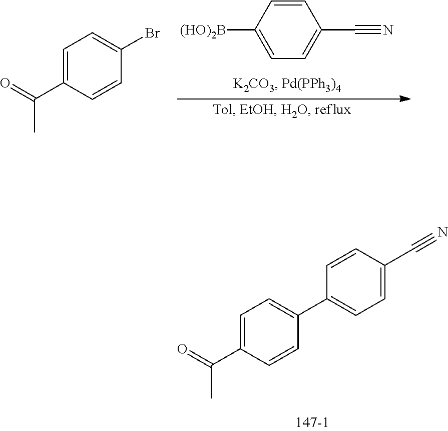

After 20 g of 1-(4-bromophenyl)ethan-1-one, 17.7 g of (4-cyanophenyl)boronic acid, 42 g of potassium carbonate (aq), and 3.5 g of Pd cat. were added to toluene and EtOH, the resulting mixture was stirred. Thereafter, after the mixture was warmed to 80° C. and reflux was maintained for 12 hours, the completion of the reaction was confirmed by TLC and HPLC, and then extraction was performed using MC and H₂O. Thereafter, an organic layer was separated and concentrated, and the resulting solid was purified using a column to obtain 17 g (yield 77%) of Intermediate 147-1.

### (2) Preparation Example 2: Synthesis of Intermediate 147-2

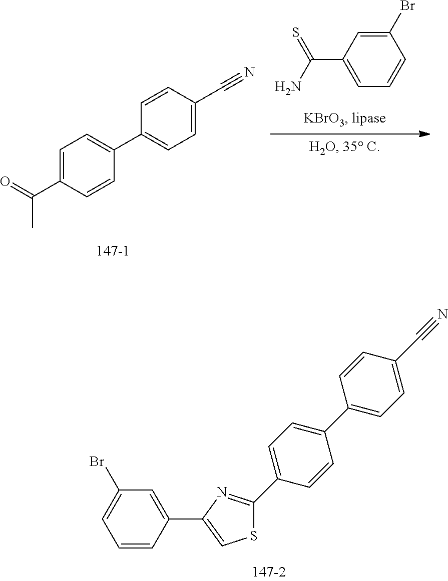 [SMILES: CC(=O)c1ccc(-c2ccc(C#N)cc2)cc1 ; NC(=S)c1cccc(Br)c1 ; N#Cc1ccc(-c2ccc(-c3nc(-c4cccc(Br)c4)cs3)cc2)cc1]

After 10 g of Intermediate 147-1, 11.3 g of 3-bromobenzothioamide, 38.5 g of KBrO₃, and 10 mol % of lipase were added to H₂O at 35° C., the resulting mixture was stirred for 10 minutes. After the completion of the reaction was confirmed by TLC and HPLC, extraction was performed with MC and H₂O to concentrate an organic layer. Thereafter, the solid was solidified with EtOH to obtain 19 g (yield 59%) of Intermediate 147-2.

### (3) Preparation Example 3. Synthesis of Intermediate 147-3

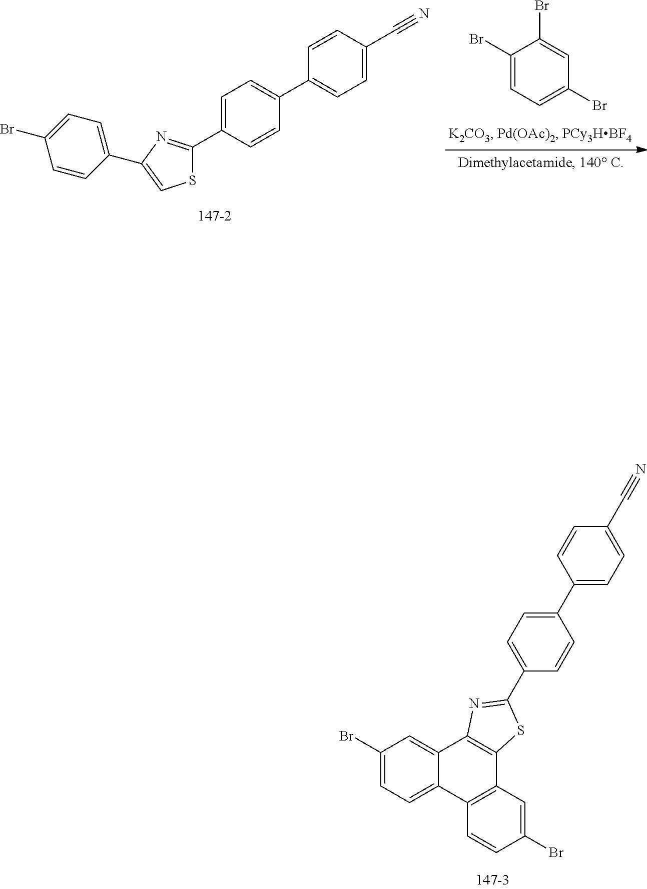 [SMILES: N#Cc1ccc(-c2ccc(-c3nc(-c4ccc(Br)cc4)cs3)cc2)cc1 ; Brc1ccc(Br)c(Br)c1 ; N#Cc1ccc(-c2ccc(-c3nc4c5cc(Br)ccc5c5ccc(Br)cc5c4s3)cc2)cc1]

After 19 g of Intermediate 147-2, 10.3 g of 1,2,4-tribromobenzene, 18.4 g of K₂CO₃, 0.4 g of Pd cat., and Pcy₃H BF₄ were added to dimethylacetamide, the resulting mixture was stirred. After the mixture was warmed to 140° C. and heated for 14 hours, the completion of the reaction was confirmed by TLC and HPLC, and then the resulting product was cooled and filtered. Thereafter, the filtered product was purified using a silica gel column and then recrystallized to obtain 5.3 g (yield 20%) of Intermediate 147-3.

### (4) Preparation Example 4: Synthesis of Compound 147

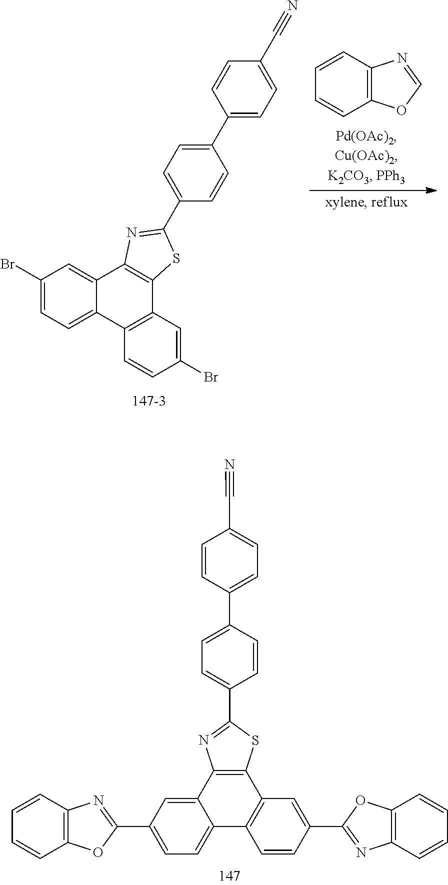

After 5 g of Intermediate 147-3, 4.4 g of benzo[d]oxazole, 20.6 g of K₂CO₃, 0.04 g of Pd cat. 0.6 g of Cu(OAc)₂, and 2.3 g of PPh₃ were added to xylene, the resulting mixture was stirred. After the mixture was warmed to 160° C. and heated for 12 hours, the completion of the reaction was confirmed by TLC and HPLC, and then the resulting product was cooled and filtered. Thereafter, the filtered product was purified using a silica gel column and then recrystallized to obtain 3 g (yield 50%) of Compound 147.

LC/MS: m/z=647[(M)+]

### Synthesis Example 5: Synthesis of Compound 251

### (1) Preparation Example 1: Synthesis of Intermediate 251-1

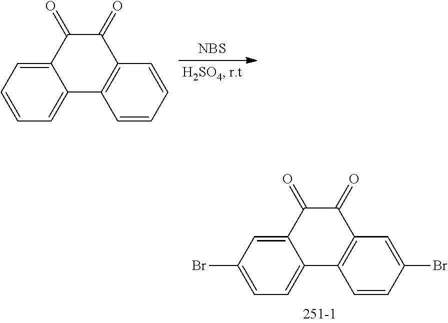

After 100 g of N-bromosuccinimide was slowly added to 1200 mL of 98% sulfuric acid at 0° C., the resulting mixture was stirred for 10 minutes, and then 50 g of phenanthrene-9,10-dione was added, and the resulting mixture was stirred at room temperature for 24 hours. After the completion of the reaction was confirmed by TLC and HPLC, the reaction solution was added to ice water and the resulting mixture was stirred. Thereafter, the mixture was filtered to obtain 75 g of orange Intermediate 251-1.

### (2) Preparation Example 2: Synthesis of Intermediate 251-2

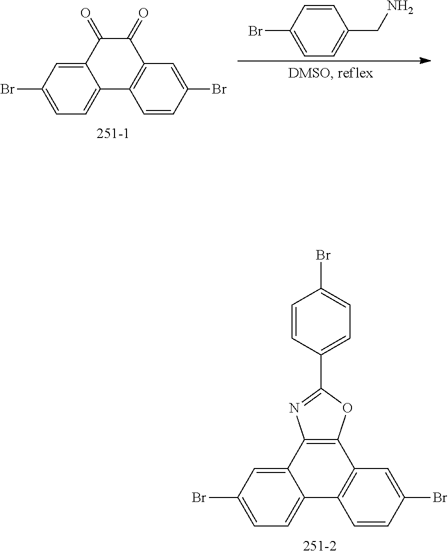 [SMILES: O=C1C(=O)c2cc(Br)ccc2-c2ccc(Br)cc21 ; NCc1ccc(Br)cc1 ; Brc1ccc(-c2nc3c4cc(Br)ccc4c4ccc(Br)cc4c3o2)cc1]

After 10 g of Intermediate 251-1 and 5.1 g of (4-bromophenyl)methanamine were added to 300 mL of DMSO, the resulting mixture was warmed to 200° C., and stirred for 24 hours. After the reaction was completed by TLC and HPLC, the resulting product was cooled to room temperature and solidified by adding MeOH. Thereafter, the solidified product was filtered and thoroughly washed with MeOH to obtain 7 g of Intermediate 251-2 (a yellow solid).

### (3) Preparation Example 3: Synthesis of Compound 251

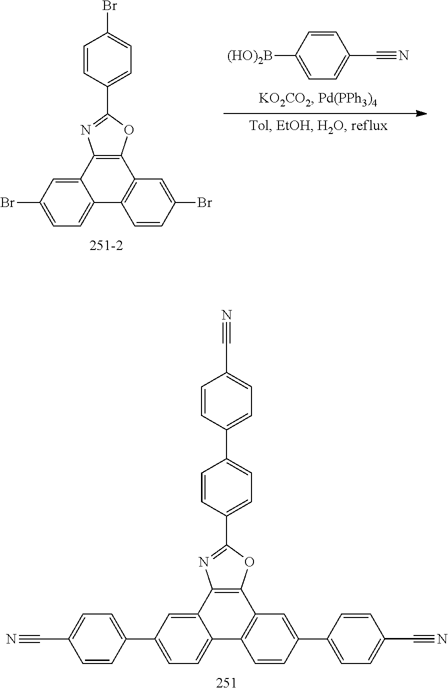 [SMILES: Brc1ccc(-c2nc3c4cc(Br)ccc4c4ccc(Br)cc4c3o2)cc1 ; N#Cc1ccc(B(O)O)cc1 ; N#Cc1ccc(-c2ccc(-c3nc4c5cc(-c6ccc(C#N)cc6)ccc5c5ccc(-c6ccc(C#N)cc6)cc5c4o3)cc2)cc1]

After 7 g of Intermediate 251-2, 7 g of (4-cyanophenyl)boronic acid, and 1.36 g of Pd cat. were added to toluene, 17 g of potassium carbonate (an aqueous solution) and ethanol were added, and the resulting mixture was warmed to 80° C. and stirred for 12 hours. After the reaction was completed by TLC and HPLC, the resulting product was cooled to room temperature, filtered, and then recrystallized with monochlorobenzene to obtain 3 g of Compound 251 (a pale yellow solid).

LC/MS: m/z=599[(M)+]

### Synthesis Example 6: Synthesis of Compound 286

### (1) Preparation Example 1: Synthesis of Compound 286

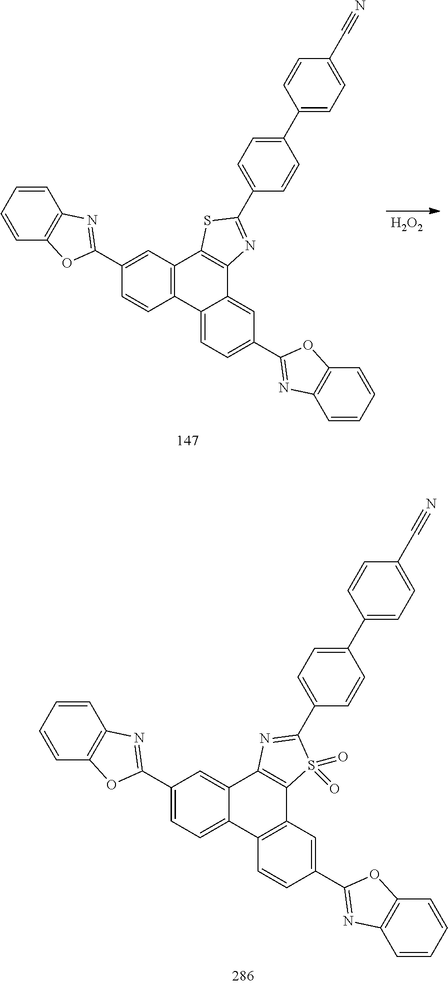 [SMILES: N#Cc1ccc(-c2ccc(-c3nc4c5cc(-c6nc7ccccc7o6)ccc5c5ccc(-c6nc7ccccc7o6)cc5c4s3)cc2)cc1 ; "H2O2" ; N#Cc1ccc(-c2ccc(C3=Nc4c(c5cc(-c6nc7ccccc7o6)ccc5c5ccc(-c6nc7ccccc7o6)cc45)S3(=O)=O)cc2)cc1]

After 1 g of Compound 147 was added to H₂O₂, the resulting mixture was stirred for 10 hours, and then the reaction was completed by confirmation by TLC and HPLC. Thereafter, the resulting product was filtered and recrystallized with MCB, and then 0.9 g (yield 86%) of Compound 286 was obtained.

LC/MS: m/z=679[(M)+]

### Synthesis Example 7: Synthesis of Compound 287

### (1) Preparation Example 1: Synthesis of Compound 287

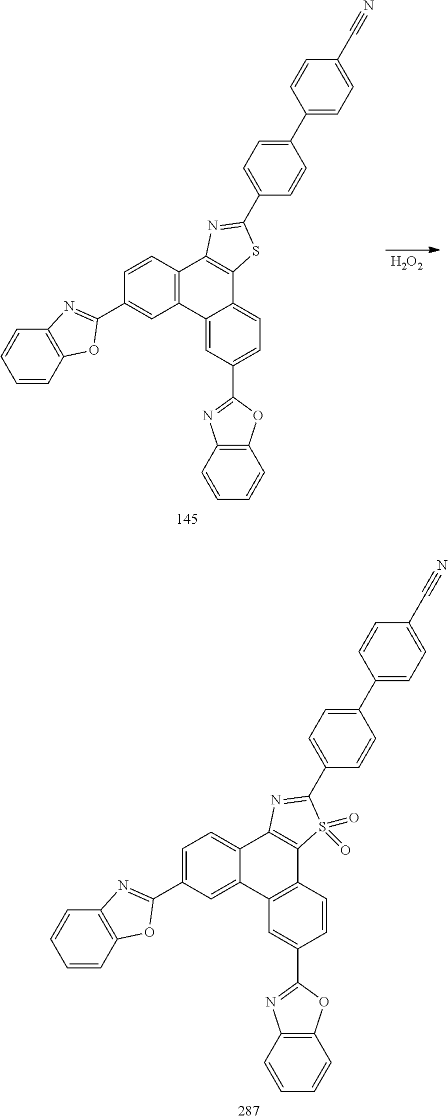 [SMILES: N#Cc1ccc(-c2ccc(-c3nc4c5ccc(-c6nc7ccccc7o6)cc5c5cc(-c6nc7ccccc7o6)ccc5c4s3)cc2)cc1 ; "H2O2" ; N#Cc1ccc(-c2ccc(C3=Nc4c(c5ccc(-c6nc7ccccc7o6)cc5c5cc(-c6nc7ccccc7o6)ccc45)S3(=O)=O)cc2)cc1]

After 1 g of Compound 145 was added to H₂O₂, the resulting mixture was stirred for 10 hours, and then the reaction was completed by confirmation by TLC and HPLC. Thereafter, the resulting product was filtered and recrystallized with MCB, and then 0.85 g (yield 81%) of Compound 287 was obtained.

LC/MS: m/z=679[(M)+]

### Synthesis Example 8: Synthesis of Compound 288

### (1) Preparation Example 1: Synthesis of Intermediate 288-1

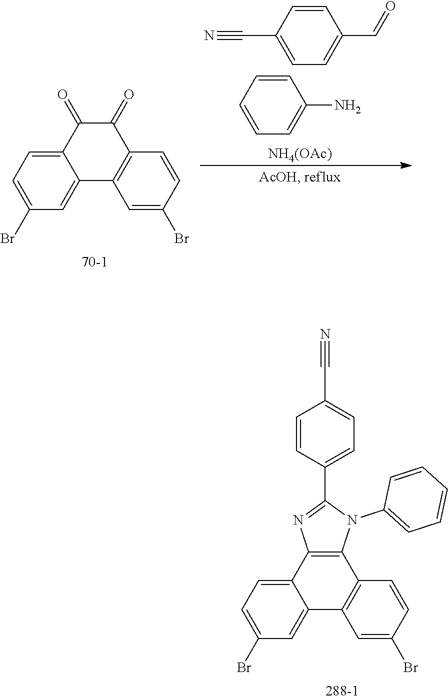

After 50 g of Intermediate 70-1, 64 g of aniline, 17.9 g of 4-formylbenzonitrile and 70.2 g of 60% ammonium acetate were added to AcOH, the resulting mixture was stirred. Thereafter, the mixture was heated to 110° C. and stirred for 4 hours, and then the reaction was completed by confirmation by TLC and HPLC. After cooling, the reaction solution was added to H₂O, and the solid was filtered and then purified using a silica gel column to obtain 51 g (yield 67%) of Intermediate 288-1.

### (2) Preparation Example 2. Synthesis of Compound 288

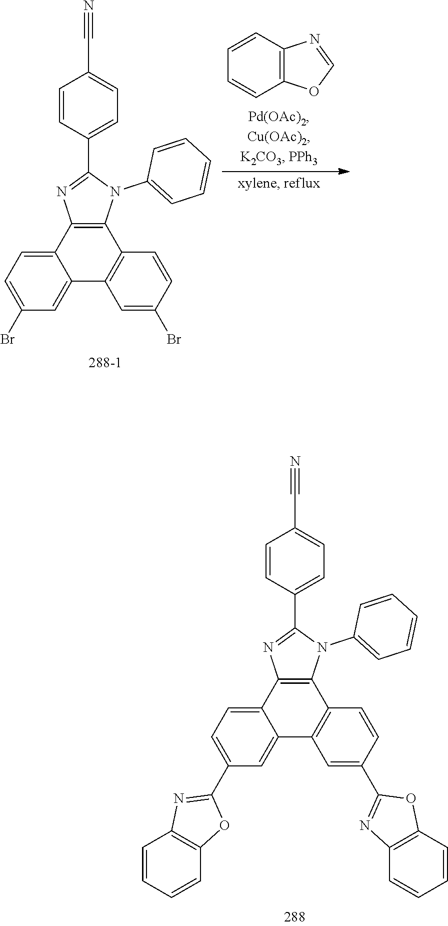 [SMILES: N#Cc1ccc(-c2nc3c4ccc(Br)cc4c4cc(Br)ccc4c3n2-c2ccccc2)cc1 ; c1ccc2ocnc2c1 ; N#Cc1ccc(-c2nc3c4ccc(-c5nc6ccccc6o5)cc4c4cc(-c5nc6ccccc6o5)ccc4c3n2-c2ccccc2)cc1]

After 10 g of Intermediate 288-1, 8.6 g of benzo[d]oxazole, 40 g of K₂CO₃, 0.32 g of Pd cat., 5.3 g of Cu(OAc)₂, and 4.7 g of PPh₃ were added to xylene, the resulting mixture was stirred. After the mixture was warmed to 160° C. and heated for 12 hours, the completion of the reaction was confirmed by TLC and HPLC, and then the resulting product was cooled and filtered. Thereafter, the filtered product was purified using a silica gel column and then recrystallized to obtain 6 g (yield 53%) of Compound 288.

LC/MS: m/z=630[(M)+]

### Synthesis Example 9: Synthesis of Compound 289

### (1) Preparation Example 1: Synthesis of Intermediate 289-1

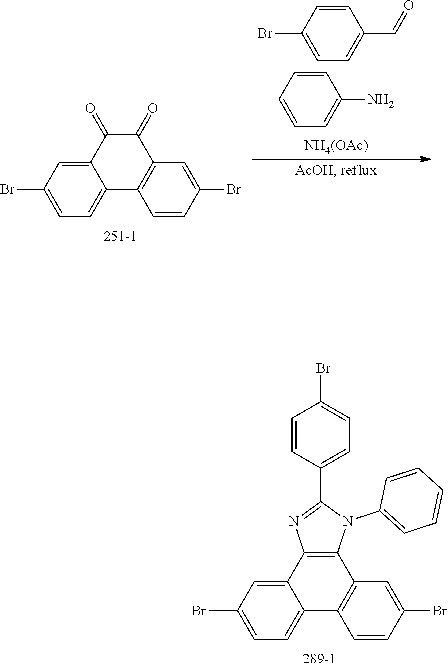

After 50 g of Intermediate 251-1, 64 g of aniline, 25.3 g of 4-bromoaldehyde, and 70.2 g of 60% ammonium acetate were added to AcOH, the resulting mixture was stirred. Thereafter, the mixture was heated to 110° C. and stirred for 4 hours, and then the reaction was completed by confirmation by TLC and HPLC. After cooling, the reaction solution was added to H₂O, and the solid was filtered and then purified using a silica gel column to obtain 63 g (yield 76%) of Intermediate 289-1.

### (2) Preparation Example 2: Synthesis of Compound 289

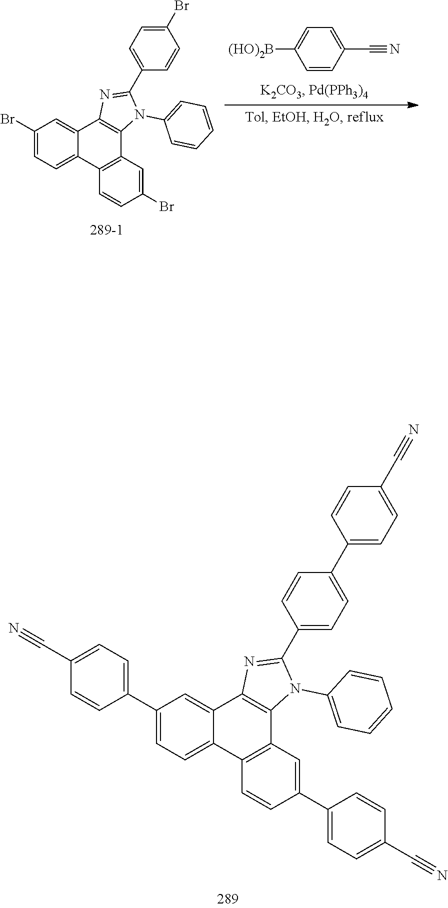 [SMILES: Brc1ccc(-c2nc3c4cc(Br)ccc4c4ccc(Br)cc4c3n2-c2ccccc2)cc1 ; N#Cc1ccc(B(O)O)cc1 ; N#Cc1ccc(-c2ccc(-c3nc4c5cc(-c6ccc(C#N)cc6)ccc5c5ccc(-c6ccc(C#N)cc6)cc5c4n3-c3ccccc3)cc2)cc1]

After 10 g of Intermediate 289-1, 9.7 g of (4-cyanophenyl)boronic acid, and 1.52 g of Pd cat. were added to toluene, 36.4 g of potassium carbonate (an aqueous solution) and ethanol were added, and the resulting mixture was warmed to 80° C. and stirred for 12 hours. After the reaction was completed by TLC and HPLC, the resulting product was cooled to room temperature, filtered, and then recrystallized with monochlorobenzene to obtain 6.3 g (yield 57%) of Compound 289.

LC/MS: m/z=674[(M)+]

### Synthesis Example 10: Synthesis of Compound 320

### (1) Preparation Example 1: Synthesis of Intermediate 320-1

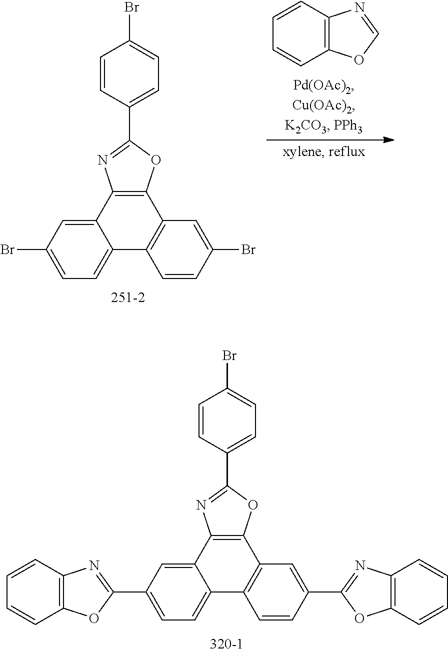 [SMILES: Brc1ccc(-c2nc3c4cc(Br)ccc4c4ccc(Br)cc4c3o2)cc1 ; c1ccc2ocnc2c1 ; Brc1ccc(-c2nc3c4cc(-c5nc6ccccc6o5)ccc4c4ccc(-c5nc6ccccc6o5)cc4c3o2)cc1]

After 10 g of Intermediate 251-2, 9 g of benzo[d]oxazole, 9 g of K₂CO₃, 0.1 g of Pd cat., 1.4 g of Cu(OAc)₂, and 5 g of PPh₃ were added to xylene, the resulting mixture was stirred. After the mixture was warmed to 160° C. and heated for 12 hours, the completion of the reaction was confirmed by TLC and HPLC, and then the resulting product was cooled and filtered. Thereafter, the filtered product was purified using a silica gel column to obtain 4.8 g (yield 42%) of Intermediate 320-1.

### (2) Preparation Example 2: Synthesis of Compound 320

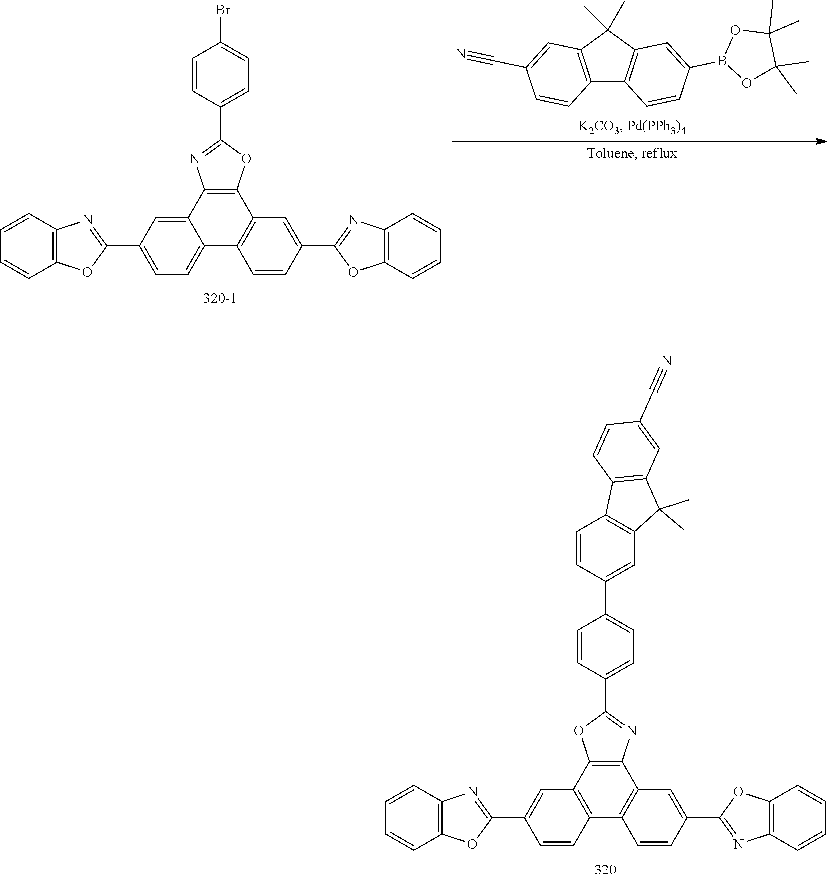 [SMILES: Brc1ccc(-c2nc3c4cc(-c5nc6ccccc6o5)ccc4c4ccc(-c5nc6ccccc6o5)cc4c3o2)cc1 ; CC1(C)c2cc(C#N)ccc2-c2ccc(B3OC(C)(C)C(C)(C)O3)cc21 ; CC1(C)c2cc(C#N)ccc2-c2ccc(-c3ccc(-c4nc5c6cc(-c7nc8ccccc8o7)ccc6c6ccc(-c7nc8ccccc8o7)cc6c5o4)cc3)cc21]

After 10 g of Intermediate 19-2 and 6.8 g of 9H-fluorene-2-carbonitrile, 9,9-dimethyl-7-(4,4,5,5-tetramethyl-1,3,2-dioxaborolan-2-yl)- (ACI) were added to toluene, 6.8 g of K₂CO₃ (aq), 1 g of Pd(PPh₃)₄, and EtOH were added, and then the resulting mixture was stirred. Thereafter, the mixture was warmed to 80° C. and maintained for 4 hours. After the completion of the reaction was confirmed by TLC and HPLC, the resulting product was cooled, extraction was performed with MC and then an organic layer was concentrated. Thereafter, the resulting product was purified using a silica gel column and then crystallized to obtain 7.1 g (yield 58%) of Compound 320.

### Synthesis Example 11: Synthesis of Compound 513

### (1) Preparation Example 1: Synthesis of Intermediate 513-1

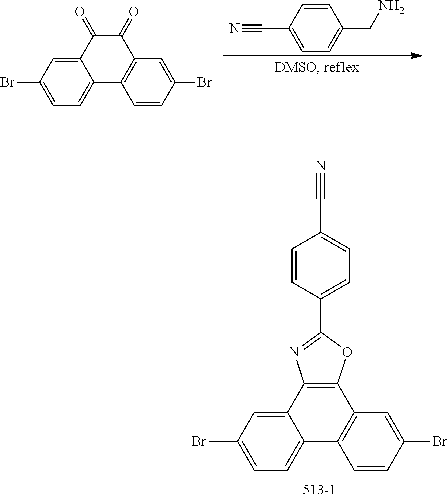 [SMILES: O=C1C(=O)c2cc(Br)ccc2-c2ccc(Br)cc21 ; N#Cc1ccc(CN)cc1 ; N#Cc1ccc(-c2nc3c4cc(Br)ccc4c4ccc(Br)cc4c3o2)cc1]

After 10 g of 2,7-dibromo-9,10-phenanthrenedione and 3.6 g of 4-(aminomethyl)benzonitrile were added to 300 mL of DMSO, the resulting mixture was warmed to 200° C., and stirred for 24 hours. After the reaction was completed by TLC and HPLC, the resulting product was cooled to room temperature and solidified by adding MeOH. Thereafter, the solidified product was filtered and thoroughly washed with MeOH to obtain 6.6 g (yield 51%) of Intermediate 513-1.

### (2) Preparation Example 2: Synthesis of Compound 513

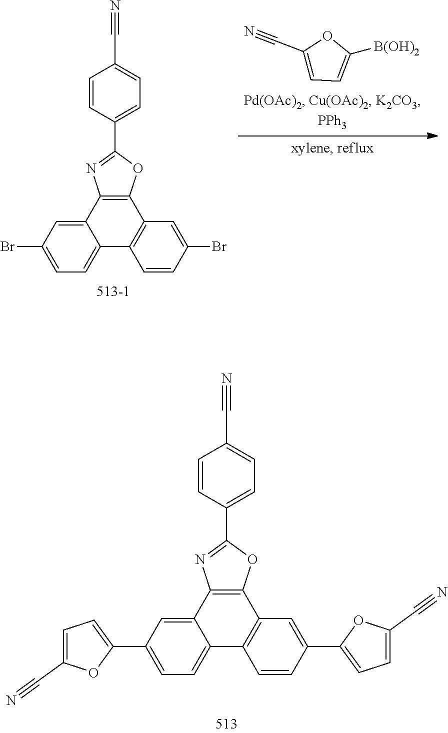 [SMILES: N#Cc1ccc(-c2nc3c4cc(Br)ccc4c4ccc(Br)cc4c3o2)cc1 ; N#Cc1ccc(B(O)O)o1 ; N#Cc1ccc(-c2nc3c4cc(-c5ccc(C#N)o5)ccc4c4ccc(-c5ccc(C#N)o5)cc4c3o2)cc1]

After 10 g of Intermediate 513-1, 11.5 g of (5-cyano-2-furyl)boronic acid, 11.6 g of K₂CO₃, 0.1 g of Pd cat., 1.5 g of Cu(OAc)₂, and 5.5 g of PPh₃ were added to xylene, the resulting mixture was stirred. After the mixture was warmed to 160° C. and heated for 12 hours, the completion of the reaction was confirmed by TLC and HPLC, and then the resulting product was cooled and filtered. Thereafter, the filtered product was purified using a silica gel and then recrystallized to obtain 5.1 g (yield 490%) of Compound 513.

### Experimental Example 1: Optical Properties of Compound According to Present Invention

In an experimental example according to the present invention, a quartz glass having a size of 25 mm×25 mm was cleaned. Thereafter, when the base pressure reached 1×10⁻⁶ torr or more by placing the quartz glass in a vacuum chamber, the optical properties were measured by depositing each of the compound according to the present invention and the comparative compound on a glass substrate.

### Examples 1 to 10

As a compound employed in the light efficiency improving layer provided in the organic light emitting device, each of the compounds according to the present invention shown in the following [Table 1] was deposited on a glass substrate to a thickness of 100 nm to measure a refractive index.

Quartz glass/Organic material (100 nm)

### Comparative Example 1

Optical properties were measured by manufacturing the substrate for Comparative Example 1 in the same manner as in Examples 1 to 10, except that the following [CP1] was used instead of the compounds of Examples 1 to 10.

### Experimental Example 1: Optical Properties of Compounds of Examples 1 to

The refractive indices of the substrates manufactured according to the Examples and Comparative Example were measured using ellipsometry (Elli-SE). The refractive index was measured in the blue wavelength region (450 nm), and the results are shown in the following Table 1.

TABLE 1

| Classification | Refractive index (450 nm) |
|---|---|
| Example 1 (Compound 70) | 2.42 |
| Example 2 (Compound 145) | 2.25 |
| Example 3 (Compound 147) | 2.38 |
| Example 4 (Compound 251) | 2.31 |
| Example 5 (Compound 286) | 2.27 |
| Example 6 (Compound 287) | 2.40 |
| Example 7 (Compound 288) | 2.33 |
| Example 8 (Compound 289) | 2.36 |
| Example 9 (Compound 320) | 2.39 |
| Example 10 (Compound 513) | 2.37 |
| Comparative Example 1 (CP1) | 1.70 |

As can be seen from [Table 1] above, the refractive index value of the compound according to the present invention in a wavelength band of 450 nm is remarkably higher than that of the compound of Comparative Example 1, and when the compound having such a high refractive index value according to the present invention is employed in a light efficiency improving layer provided in an organic light-emitting device, the efficiency of the device can be optimized.

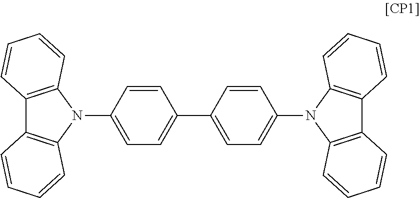 [SMILES: c1ccc2c(c1)c1ccccc1n2-c1ccc(-c2ccc(-n3c4ccccc4c4ccccc43)cc2)cc1]

### Device Examples (CPL)

In embodiments according to the present invention, an anode was patterned using an ITO glass substrate having a size of 25 mm×25 mm×0.7 mm and including Ag such that a light-emitting area had a size of 2 mm×2 mm, and then washed. After the patterned ITO substrate was mounted in a vacuum chamber, organic materials and metals were deposited in the following structure on the substrate at a process pressure of 1×10⁻⁶ torr or more.

### Examples 11 to 44

After blue organic light emitting devices having the following device structure were manufactured by employing the compounds implemented according to the present invention in the light efficiency improving layer (monolayer CPL) provided in a device, luminescent and driving properties were measured.

Ag/ITO/hole injecting layer (HAT-CN, 5 nm)/hole transport layer (HT1, 100 nm)/electron blocking layer (EB1, 10 nm)/light emitting layer (20 nm)/electron transport layer (ET1:Liq, 30 nm)/LiF (1 nm)/Mg:Ag (15 nm)/light efficiency improving layer (65 nm)

On a glass substrate, [HAT-CN]was formed into a film to form a 5 nm thick hole injecting layer on an Ag-containing ITO transparent electrode. Thereafter, a 100 nm thick hole transport layer was formed into a film using [HT-1]. Thereafter, 10 nm thick electron blocking layer was formed into a film using [EB1]. Thereafter, a light-emitting layer was formed by co-depositing [BH1] and [BD1] to a thickness of 20 nm using [BH1] as a host compound and [BD1] as a dopant compound. Thereafter, an electron transport layer (50% doping of the following [ET1] compound Liq) was deposited to a thickness of 30 nm, and then LiF was formed into a film to a thickness of 1 nm to form an electron injection layer. Thereafter, Mg and Ag were formed into a film at a ratio of 1:9 to a thickness of 15 nm to form a cathode.

In addition, as a light efficiency improving layer (capping layer), the compound of [Formula I] according to the present invention shown in the following [Table 2] was formed into a film to a thickness of 65 nm to manufacture an organic light-emitting device.

### Device Comparative Example 2

An organic light-emitting device for Device Comparative Example 2 was manufactured in the same manner as in the device structures of Examples 11 to 44, except that the light efficiency improving layer was not provided.

### Device Comparative Example 3

An organic light emitting device for Device Comparative Example 3 was manufactured in the same manner in the device structures of Examples 11 to 44, except that the following [CP1] was used instead of the compound according to the present invention as the light efficiency improving layer compound.

### Experimental Example 2: Light-Emitting Properties of Device Examples 11 to 44

For the organic light-emitting devices manufactured by the Examples and the Comparative Examples, driving voltage, current efficiency and color coordinate were measured using a source meter (Model237, Keithley) and a luminance meter (PR-650, Photo Research), and the result values based on 1,000 nits are shown in the following [Table 2].

TABLE 2

| | Light efficiency | | | | |
|---|---|---|---|---|---|
| Example | improving layer | V | cd/A | CIEx | CIEy |
| 11 | Compound 11 | 3.68 | 8.25 | 0.1415 | 0.1325 |
| 12 | Compound 15 | 3.53 | 8.34 | 0.1341 | 0.1385 |
| 13 | Compound 27 | 3.84 | 8.46 | 0.1353 | 0.1379 |
| 14 | Compound 36 | 3.75 | 8.72 | 0.1392 | 0.1345 |
| 15 | Compound 41 | 3.50 | 8.59 | 0.145 | 0.1303 |
| 16 | Compound 45 | 3.58 | 8.50 | 0.1337 | 0.1387 |
| 17 | Compound 48 | 3.61 | 8.23 | 0.1374 | 0.1361 |
| 18 | Compound 62 | 3.80 | 8.37 | 0.1445 | 0.1308 |
| 19 | Compound 66 | 3.69 | 8.42 | 0.1423 | 0.1318 |
| 20 | Compound 70 | 3.54 | 8.66 | 0.1401 | 0.1337 |
| 21 | Compound 81 | 3.78 | 8.30 | 0.1395 | 0.1342 |
| 22 | Compound 100 | 3.60 | 8.74 | 0.1356 | 0.1374 |
| 23 | Compound 132 | 3.71 | 8.48 | 0.1383 | 0.1351 |
| 24 | Compound 145 | 3.66 | 8.31 | 0.1325 | 0.1393 |
| 25 | Compound 148 | 3.90 | 8.78 | 0.1436 | 0.1311 |
| 26 | Compound 156 | 3.76 | 8.22 | 0.1362 | 0.1367 |
| 27 | Compound 160 | 3.57 | 8.56 | 0.1455 | 0.1301 |
| 28 | Compound 179 | 3.72 | 8.64 | 0.142 | 0.1321 |
| 29 | Compound 187 | 3.52 | 8.40 | 0.1378 | 0.1359 |
| 30 | Compound 197 | 3.63 | 8.71 | 0.144 | 0.1309 |
| 31 | Compound 219 | 3.62 | 8.36 | 0.1332 | 0.1392 |
| 32 | Compound 228 | 3.85 | 8.62 | 0.1398 | 0.1339 |
| 33 | Compound 236 | 3.73 | 8.52 | 0.1321 | 0.1398 |
| 34 | Compound 240 | 3.59 | 8.69 | 0.1406 | 0.1334 |
| 35 | Compound 251 | 3.55 | 8.29 | 0.1387 | 0.1348 |
| 36 | Compound 253 | 3.67 | 8.41 | 0.1431 | 0.1314 |
| 37 | Compound 286 | 3.56 | 8.60 | 0.1412 | 0.1328 |
| 38 | Compound 287 | 3.81 | 8.27 | 0.1369 | 0.1362 |
| 39 | Compound 288 | 3.87 | 8.70 | 0.1348 | 0.1381 |
| 40 | Compound 289 | 3.74 | 8.55 | 0.1427 | 0.1316 |
| 41 | Compound 320 | 3.55 | 8.65 | 0.1400 | 0.1339 |
| 42 | Compound 477 | 3.56 | 8.63 | 0.1403 | 0.1336 |
| 43 | Compound 513 | 3.76 | 8.54 | 0.1427 | 0.1318 |
| 44 | Compound 687 | 3.76 | 8.54 | 0.1427 | 0.1318 |
| Comparative | Not provided | 4.61 | 7.12 | 0.1497 | 0.1372 |
| Example 2 | | | | | |
| Comparative | CP1 | 4.04 | 7.97 | 0.1362 | 0.1298 |
| Example 3 | | | | | |

As can be seen in [Table 2] above, it can be confirmed that when the compound of [Formula I] according to the present invention is applied to a device as a light efficiency improving layer, the driving voltage is reduced and the current efficiency is improved compared to each of the device in the related art, which does not include a light efficiency improving layer and the device in related art, in which a compound used as a material for a light efficiency improving layer is used (Comparative Examples 2 and 3).

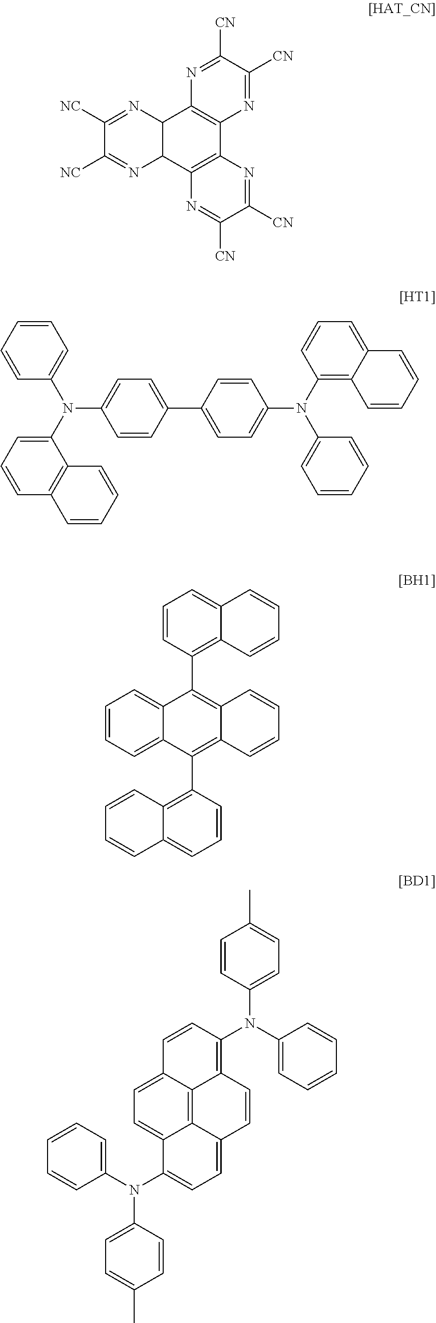

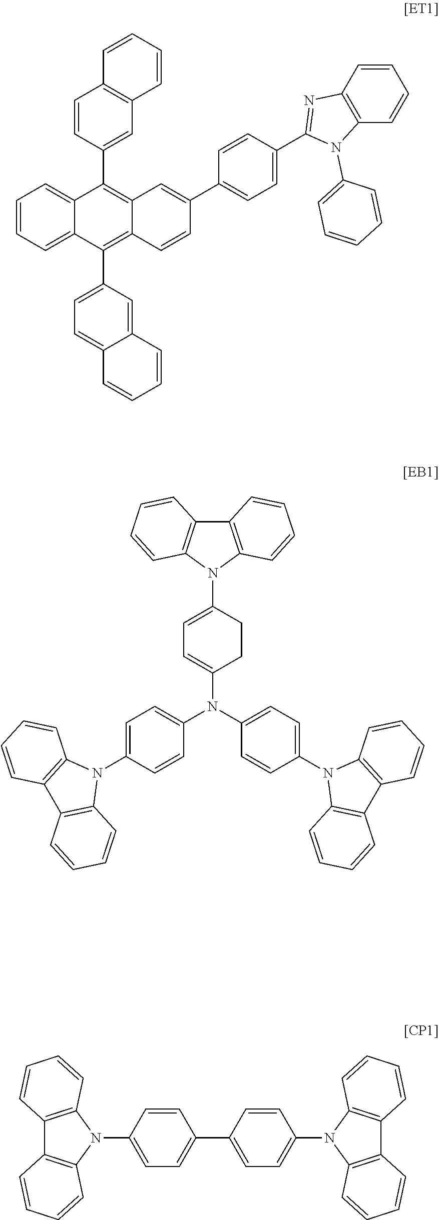 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; C1=C(N(c2ccc(-n3c4ccccc4c4ccccc43)cc2)c2ccc(-n3c4ccccc4c4ccccc43)cc2)CCC(n2c3ccccc3c3ccccc32)=C1 ; c1ccc2c(c1)c1ccccc1n2-c1ccc(-c2ccc(-n3c4ccccc4c4ccccc43)cc2)cc1]

What is claimed is:

1. An organic compound represented by [Formula I]: 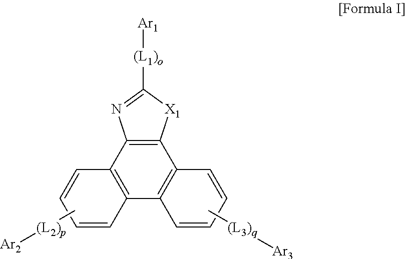
wherein, in [Formula I] above,
X₁ is any one selected from O, S, SO (sulfur monoxide), SO₂ (sulfur dioxide), NR₁, CR₂R₃, SiR₄R₅, and GeR₆R₇,
R₁ to R₇ are the same as or different from each other, and are each independently any one selected from hydrogen, deuterium, a substituted or unsubstituted alkyl group having 1 to 20 carbon atoms, a substituted or unsubstituted alkoxy group having 1 to 20 carbon atoms, a substituted or unsubstituted cycloalkyl group having 3 to 20 carbon atoms, a substituted or unsubstituted heterocycloalkyl group having 3 to 30 carbon atoms, a substituted or unsubstituted aryl group having 6 to 30 carbon atoms, a substituted or unsubstituted heteroaryl group having 2 to 50 carbon atoms, a substituted or unsubstituted silyl group, a substituted or unsubstituted germanium group, a cyano group, and a halogen group,
L₁ to L₃ are a divalent linking group, and are a direct bond or any one selected from a substituted or unsubstituted arylene group having 6 to 30 carbon atoms and a substituted or unsubstituted heteroarylene group having 2 to 50 carbon atoms,
o, p, and q are an integer from 0 to 2, and when o, p, and q are 2, a plurality of L₁ to L₃ are each the same as or different from each other,
Ar₁ to Ar₃ are the same as or different from each other, and are each independently a cyano group (CN), or any one selected from a substituted or unsubstituted aryl group having 6 to 30 carbon atoms and a substituted or unsubstituted heteroaryl group having 2 to 50 carbon atoms, and
The ‘substituted or unsubstituted’ means being substituted with one or two or more substituents selected from deuterium, a cyano group, a halogen group, a hydroxy group, a nitro group, an alkyl group, a halogenated alkyl group, an alkoxy group, a halogenated alkoxy group, a cycloalkyl group, a heterocycloalkyl group, an aryl group, a heteroaryl group, a mixed aliphatic-aromatic cyclic group, an amine group, a silyl group, and a germanium group, being substituted with a substituent to which two or more substituents among the aforementioned substituents are linked, or having no substituent.

2. The organic compound of claim 1, wherein L₁ to L₃ are a divalent linking group, and are a direct bond or a substituted or unsubstituted arylene group having 6 to 30 carbon atoms, and
Ar₁ to Ar₃ are the same as or different from each other, and are each independently a substituted or unsubstituted aryl group having 6 to 30 carbon atoms.

3. The organic compound of claim 1, wherein L₁ to L₃ are a divalent linking group, and are a direct bond or a substituted or unsubstituted phenylene group, and Ar₁ to Ar₃ are each a substituted or unsubstituted naphthyl group.

4. The organic compound of claim 1,
wherein at least one or more of Ar₁ to Ar₃ are the following [Structural Formula 1] or any one selected from the following [Structural Formula 2]: 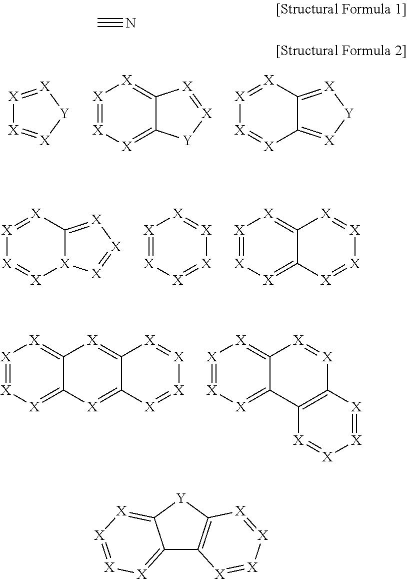
in each structural formula in [Structural Formula 2] above,
X's are the same as or different from each other, and are each independently N or CR₁₁, and Y is any one selected from O, S, NR₁₂, CR₁₃R₁₄, SiR₁₅R₁₆, and GeR₁₇R₁₈,
R₁₁ to R₁₈ are the same as or different from each other, and are each independently any one selected from hydrogen, deuterium, a cyano group, a halogen group, a substituted or unsubstituted alkyl group having 1 to 20 carbon atoms, a substituted or unsubstituted alkenyl group having 2 to 20 carbon atoms, an alkynyl group having 2 to 20 carbon atoms, a substituted or unsubstituted alkoxy group having 1 to 20 carbon atoms, a substituted or unsubstituted cycloalkyl group having 3 to 20 carbon atoms, a substituted or unsubstituted heterocycloalkyl group having 3 to 30 carbon atoms, a substituted or unsubstituted aryl group having 6 to 30 carbon atoms, a substituted or unsubstituted heteroaryl group having 2 to 50 carbon atoms, a substituted or unsubstituted amine group having 1 to 50 carbon atoms, a substituted or unsubstituted silyl group, and a substituted or unsubstituted germanium group,
the plurality of R₁₁'s are optionally linked to each other to form an alicyclic or aromatic monocyclic or polycyclic ring,
R₁₃ and R₁₄, R₁₅ and R₁₆, and R₁₇ and R₁₈ are each optionally linked to each other to form an alicyclic or aromatic monocyclic or polycyclic ring,
the carbon atoms of the formed alicyclic or aromatic monocyclic or polycyclic ring are able to be replaced by heteroatoms selected from O, S, N, P, Si, and Ge,
any one selected from R₁₁ to R₁₈ in each structural formula is bonded to L₁ to L₃ in [Formula I] above, and
the ‘substituted or unsubstituted’ means being substituted with one or two or more substituents selected from deuterium, a cyano group, a halogen group, an alkyl group, a halogenated alkyl group, an alkoxy group, a halogenated alkoxy group, a cycloalkyl group, a heterocycloalkyl group, an aryl group, a heteroaryl group, a mixed aliphatic-aromatic cyclic group, an amine group, a silyl group, and a germanium group, being substituted with a substituent to which two or more substituents among the aforementioned substituents are linked, or having no substituent.

5. The organic compound of claim 4, wherein at least one or more of Ar₁ to Ar₃ are [Structural Formula 1] or [Structural Formula 2-2] below: 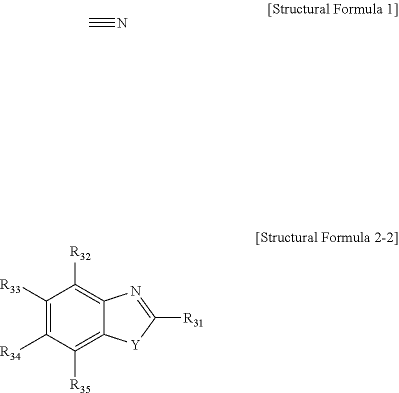
in [Structural Formula 2-2] above,
Y is any one selected from O, S, SO (sulfur monoxide), SO₂ (sulfur dioxide), and NR₂₁,
R₂₁ is any one selected from hydrogen, deuterium, a substituted or unsubstituted alkyl group having 1 to 20 carbon atoms, a substituted or unsubstituted alkoxy group having 1 to 20 carbon atoms, a substituted or unsubstituted cycloalkyl group having 3 to 20 carbon atoms, a substituted or unsubstituted heterocycloalkyl group having 3 to 30 carbon atoms, a substituted or unsubstituted aryl group having 6 to 30 carbon atoms, a substituted or unsubstituted heteroaryl group having 2 to 50 carbon atoms, a substituted or unsubstituted silyl group, a substituted or unsubstituted germanium group, a cyano group, and a halogen group,
R₃₁ to R₃₅ are the same as or different from each other, and are each independently any one selected from hydrogen, deuterium, a substituted or unsubstituted alkyl group having 1 to 20 carbon atoms, a substituted or unsubstituted alkoxy group having 1 to 20 carbon atoms, a substituted or unsubstituted cycloalkyl group having 3 to 20 carbon atoms, a substituted or unsubstituted heterocycloalkyl group having 3 to 30 carbon atoms, a substituted or unsubstituted aryl group having 6 to 30 carbon atoms, a substituted or unsubstituted heteroaryl group having 2 to 50 carbon atoms, a substituted or unsubstituted silyl group, a substituted or unsubstituted germanium group, a cyano group, and a halogen,
R₃₂ to R₃₅ are optionally each linked to each other or an adjacent substituent to additionally form an alicyclic or aromatic monocyclic or polycyclic ring,
any one of R₃₁ to R₃₅ is a moiety bonded to L₁ to L₃, and
The ‘substituted or unsubstituted’ means being substituted with one or two or more substituents selected from deuterium, a cyano group, a halogen group, a hydroxy group, a nitro group, an alkyl group, a halogenated alkyl group, an alkoxy group, a halogenated alkoxy group, a cycloalkyl group, a heterocycloalkyl group, an aryl group, a heteroaryl group, a mixed aliphatic-aromatic cyclic group, an amine group, a silyl group, and a germanium group, being substituted with a substituent to which two or more substituents among the aforementioned substituents are linked, or having no substituent.

6. The organic compound of claim 5, wherein [Structural Formula 2-2] above is any one selected from the following [Structural Formula 3]: 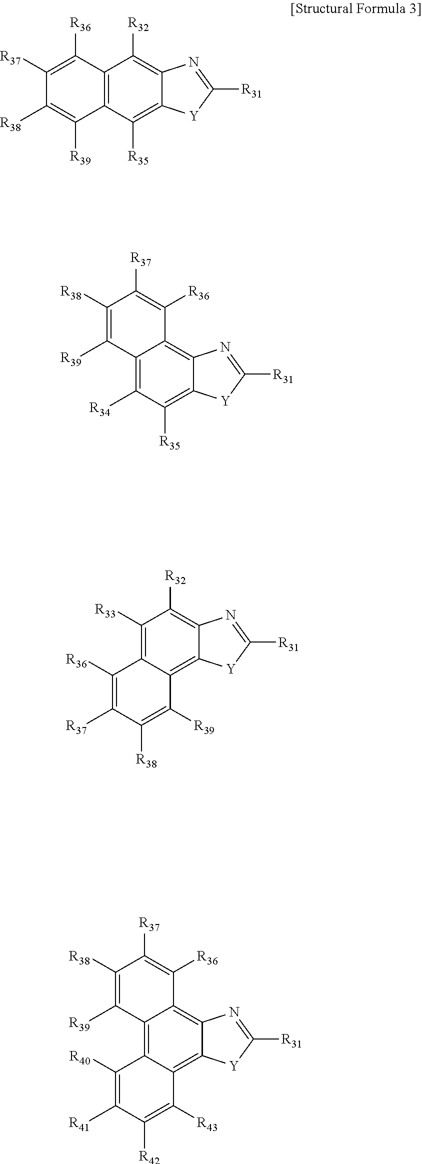
in [Structural Formula 3] above,
Y is any one selected from O, S, SO (sulfur monoxide), SO₂ (sulfur dioxide), and NR₂₁,
R₂₁ is any one selected from hydrogen, deuterium, a substituted or unsubstituted alkyl group having 1 to 20 carbon atoms, a substituted or unsubstituted alkoxy group having 1 to 20 carbon atoms, a substituted or unsubstituted cycloalkyl group having 3 to 20 carbon atoms, a substituted or unsubstituted heterocycloalkyl group having 3 to 30 carbon atoms, a substituted or unsubstituted aryl group having 6 to 30 carbon atoms, a substituted or unsubstituted heteroaryl group having 2 to 50 carbon atoms, a substituted or unsubstituted silyl group, a substituted or unsubstituted germanium group, a cyano group, and a halogen group,
R₃₁ to R₄₃ are the same as or different from each other, and are each independently any one selected from hydrogen, deuterium, a substituted or unsubstituted alkyl group having 1 to 20 carbon atoms, a substituted or unsubstituted alkoxy group having 1 to 20 carbon atoms, a substituted or unsubstituted cycloalkyl group having 3 to 20 carbon atoms, a substituted or unsubstituted heterocycloalkyl group having 3 to 30 carbon atoms, a substituted or unsubstituted aryl group having 6 to 30 carbon atoms, a substituted or unsubstituted heteroaryl group having 2 to 50 carbon atoms, a substituted or unsubstituted silyl group, a substituted or unsubstituted germanium group, a cyano group, and a halogen,
any one of R₃₁ to R₄₃ is a moiety bonded to L₁ to L₃, and
The ‘substituted or unsubstituted’ means being substituted with one or two or more substituents selected from deuterium, a cyano group, a halogen group, a hydroxy group, a nitro group, an alkyl group, a halogenated alkyl group, an alkoxy group, a halogenated alkoxy group, a cycloalkyl group, a heterocycloalkyl group, an aryl group, a heteroaryl group, a mixed aliphatic-aromatic cyclic group, an amine group, a silyl group, and a germanium group, being substituted with a substituent to which two or more substituents among the aforementioned substituents are linked, or having no substituent.

7. The organic compound of claim 4, wherein Ar₁ to Ar₃ are the same as or different from each other, and are each independently [Structural Formula 1] or [Structural Formula 2].

8. The organic compound of claim 1, wherein [Formula I] above is any one selected from the following [Compound 1] to [Compound 758]:

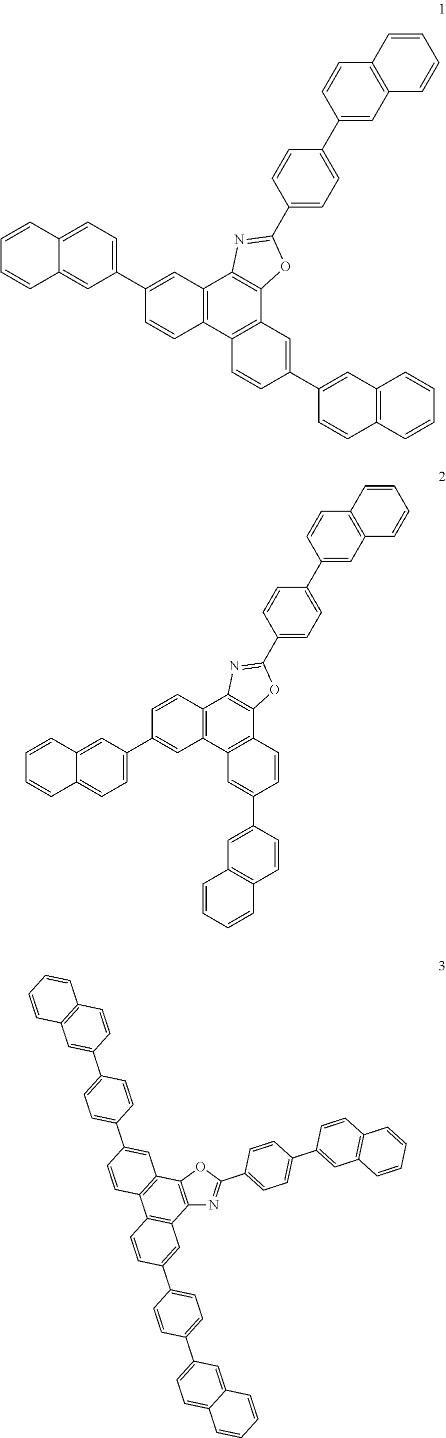 [SMILES: c1ccc2cc(-c3ccc(-c4nc5c6cc(-c7ccc8ccccc8c7)ccc6c6ccc(-c7ccc8ccccc8c7)cc6c5o4)cc3)ccc2c1 ; c1ccc2cc(-c3ccc(-c4nc5c6ccc(-c7ccc8ccccc8c7)cc6c6cc(-c7ccc8ccccc8c7)ccc6c5o4)cc3)ccc2c1 ; c1ccc2cc(-c3ccc(-c4ccc5c6ccc(-c7ccc(-c8ccc9ccccc9c8)cc7)cc6c6oc(-c7ccc(-c8ccc9ccccc9c8)cc7)nc6c5c4)cc3)ccc2c1]

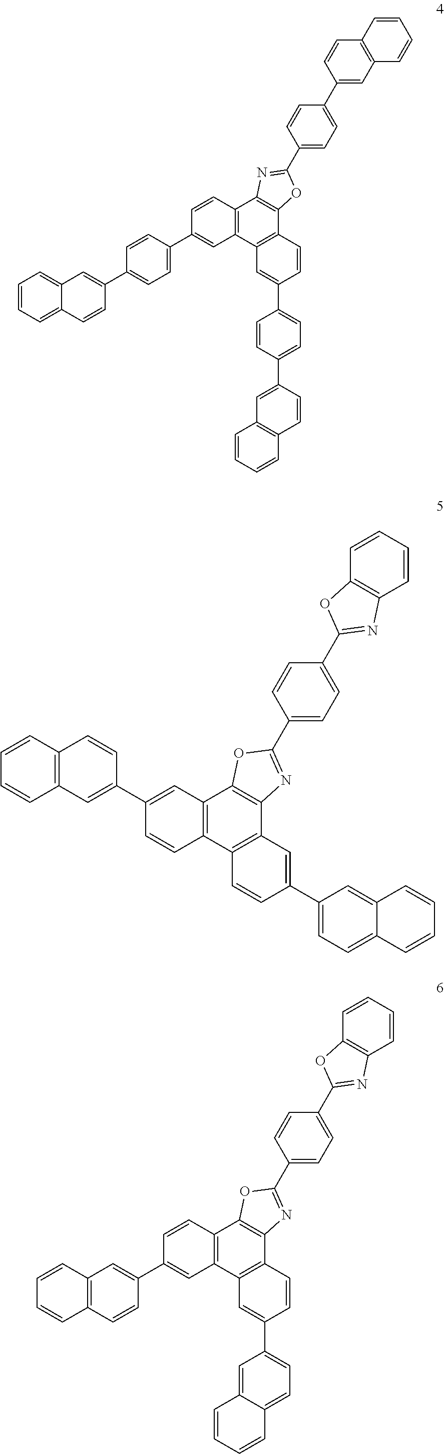 [SMILES: c1ccc2cc(-c3ccc(-c4ccc5c(c4)c4cc(-c6ccc(-c7ccc8ccccc8c7)cc6)ccc4c4oc(-c6ccc(-c7ccc8ccccc8c7)cc6)nc54)cc3)ccc2c1 ; c1ccc2cc(-c3ccc4c5ccc(-c6ccc7ccccc7c6)cc5c5oc(-c6ccc(-c7nc8ccccc8o7)cc6)nc5c4c3)ccc2c1 ; c1ccc2cc(-c3ccc4c(c3)c3cc(-c5ccc6ccccc6c5)ccc3c3oc(-c5ccc(-c6nc7ccccc7o6)cc5)nc43)ccc2c1]

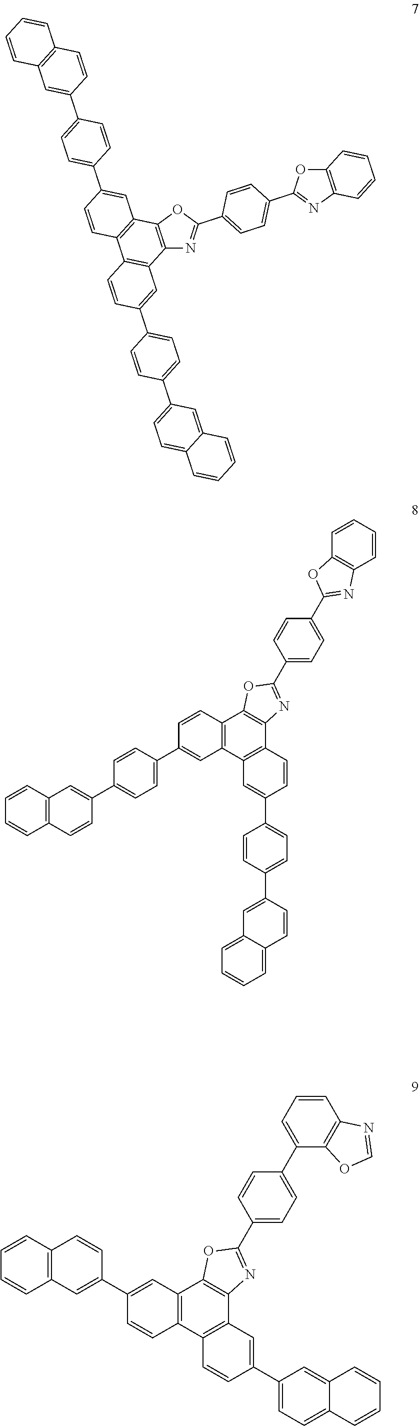 [SMILES: c1ccc2cc(-c3ccc(-c4ccc5c6ccc(-c7ccc(-c8ccc9ccccc9c8)cc7)cc6c6oc(-c7ccc(-c8nc9ccccc9o8)cc7)nc6c5c4)cc3)ccc2c1 ; c1ccc2cc(-c3ccc(-c4ccc5c(c4)c4cc(-c6ccc(-c7ccc8ccccc8c7)cc6)ccc4c4oc(-c6ccc(-c7nc8ccccc8o7)cc6)nc54)cc3)ccc2c1 ; c1ccc2cc(-c3ccc4c5ccc(-c6ccc7ccccc7c6)cc5c5oc(-c6ccc(-c7cccc8ncoc78)cc6)nc5c4c3)ccc2c1]

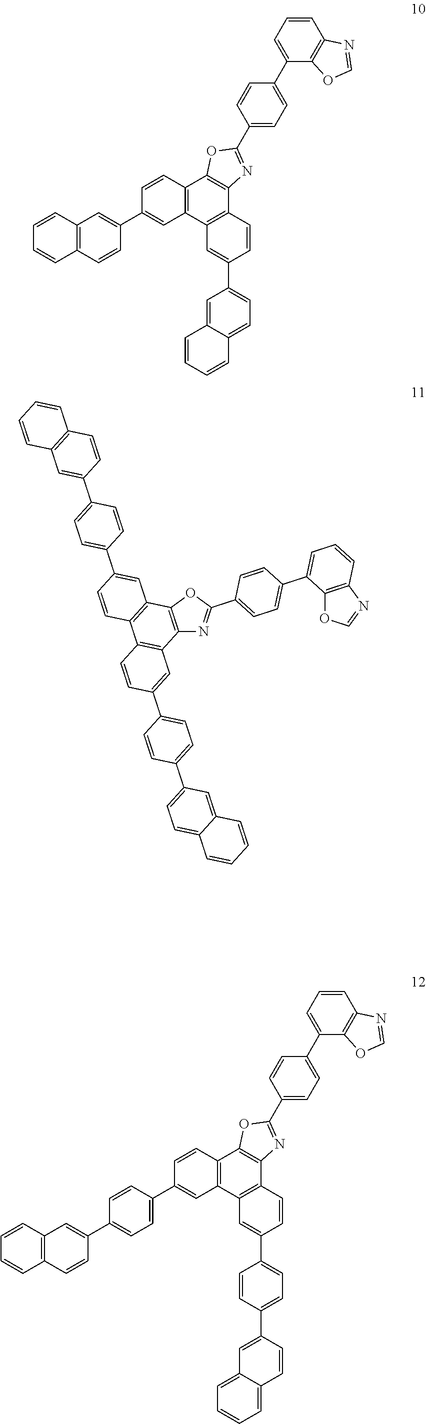 [SMILES: c1ccc2cc(-c3ccc4c(c3)c3cc(-c5ccc6ccccc6c5)ccc3c3oc(-c5ccc(-c6cccc7ncoc67)cc5)nc43)ccc2c1 ; c1ccc2cc(-c3ccc(-c4ccc5c6ccc(-c7ccc(-c8ccc9ccccc9c8)cc7)cc6c6oc(-c7ccc(-c8cccc9ncoc89)cc7)nc6c5c4)cc3)ccc2c1 ; c1ccc2cc(-c3ccc(-c4ccc5c(c4)c4cc(-c6ccc(-c7ccc8ccccc8c7)cc6)ccc4c4oc(-c6ccc(-c7cccc8ncoc78)cc6)nc54)cc3)ccc2c1]

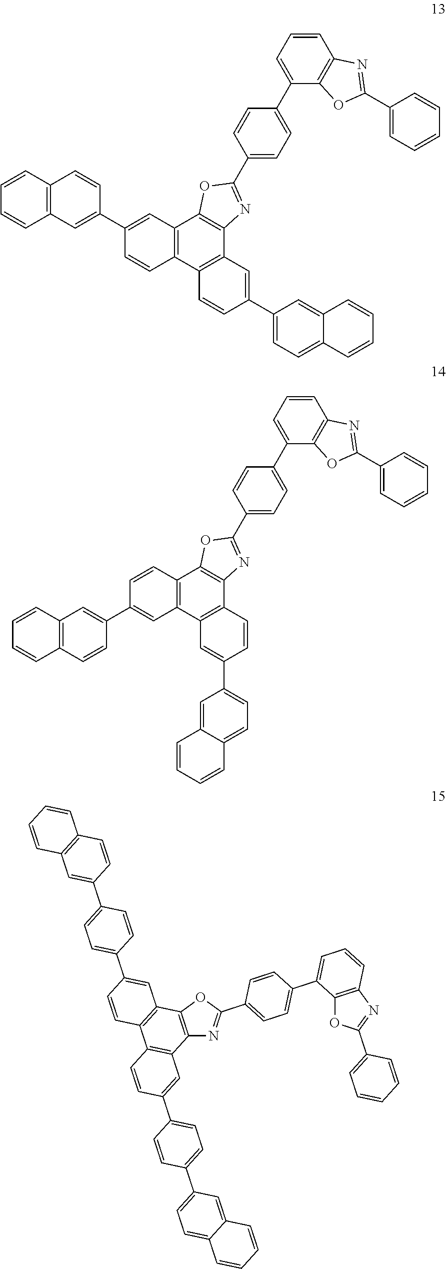 [SMILES: c1ccc(-c2nc3cccc(-c4ccc(-c5nc6c7cc(-c8ccc9ccccc9c8)ccc7c7ccc(-c8ccc9ccccc9c8)cc7c6o5)cc4)c3o2)cc1 ; c1ccc(-c2nc3cccc(-c4ccc(-c5nc6c7ccc(-c8ccc9ccccc9c8)cc7c7cc(-c8ccc9ccccc9c8)ccc7c6o5)cc4)c3o2)cc1 ; c1ccc(-c2nc3cccc(-c4ccc(-c5nc6c7cc(-c8ccc(-c9ccc%10ccccc%10c9)cc8)ccc7c7ccc(-c8ccc(-c9ccc%10ccccc%10c9)cc8)cc7c6o5)cc4)c3o2)cc1]

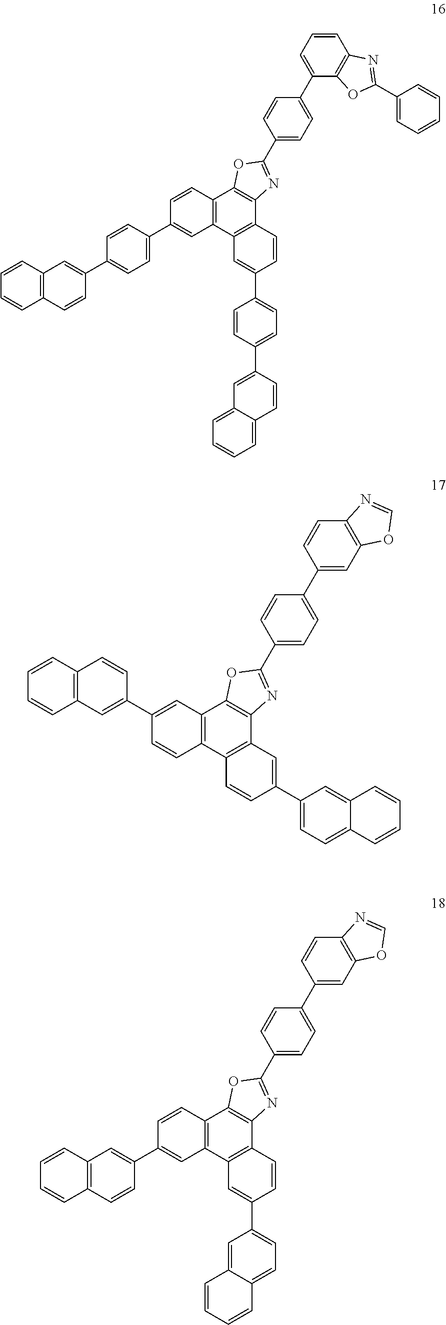 [SMILES: c1ccc(-c2nc3cccc(-c4ccc(-c5nc6c7ccc(-c8ccc(-c9ccc%10ccccc%10c9)cc8)cc7c7cc(-c8ccc(-c9ccc%10ccccc%10c9)cc8)ccc7c6o5)cc4)c3o2)cc1 ; c1ccc2cc(-c3ccc4c5ccc(-c6ccc7ccccc7c6)cc5c5oc(-c6ccc(-c7ccc8ncoc8c7)cc6)nc5c4c3)ccc2c1 ; c1ccc2cc(-c3ccc4c(c3)c3cc(-c5ccc6ccccc6c5)ccc3c3oc(-c5ccc(-c6ccc7ncoc7c6)cc5)nc43)ccc2c1]

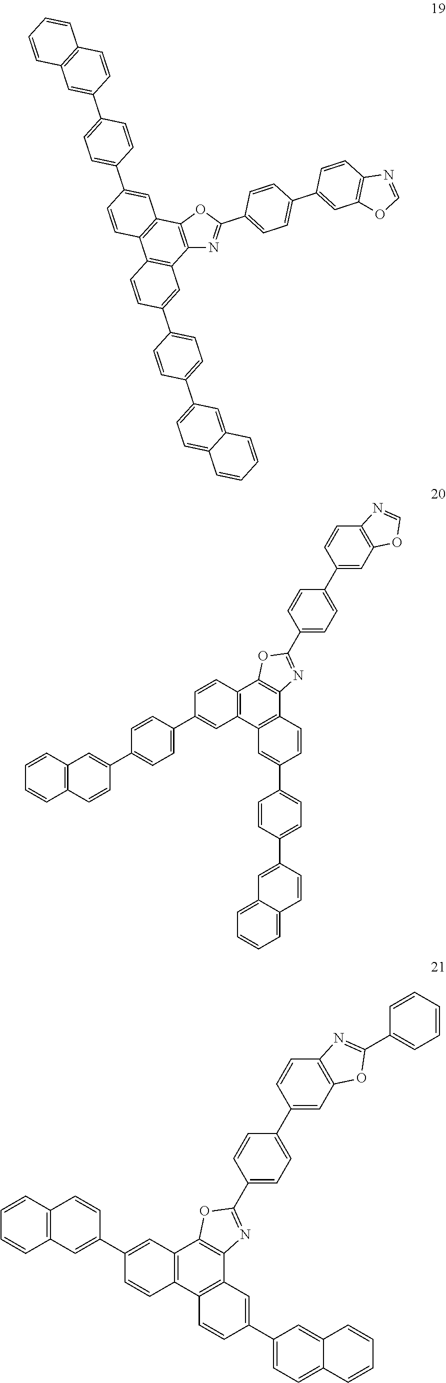 [SMILES: c1ccc2cc(-c3ccc(-c4ccc5c6ccc(-c7ccc(-c8ccc9ccccc9c8)cc7)cc6c6oc(-c7ccc(-c8ccc9ncoc9c8)cc7)nc6c5c4)cc3)ccc2c1 ; c1ccc2cc(-c3ccc(-c4ccc5c(c4)c4cc(-c6ccc(-c7ccc8ccccc8c7)cc6)ccc4c4oc(-c6ccc(-c7ccc8ncoc8c7)cc6)nc54)cc3)ccc2c1 ; c1ccc(-c2nc3ccc(-c4ccc(-c5nc6c7cc(-c8ccc9ccccc9c8)ccc7c7ccc(-c8ccc9ccccc9c8)cc7c6o5)cc4)cc3o2)cc1]

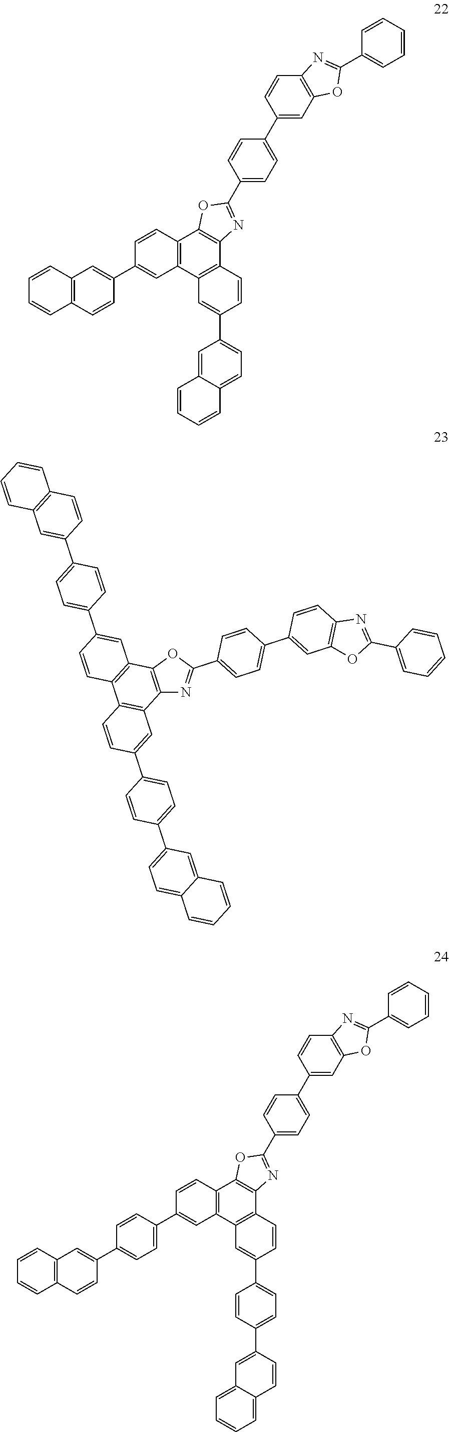 [SMILES: c1ccc(-c2nc3ccc(-c4ccc(-c5nc6c7ccc(-c8ccc9ccccc9c8)cc7c7cc(-c8ccc9ccccc9c8)ccc7c6o5)cc4)cc3o2)cc1 ; c1ccc(-c2nc3ccc(-c4ccc(-c5nc6c7cc(-c8ccc(-c9ccc%10ccccc%10c9)cc8)ccc7c7ccc(-c8ccc(-c9ccc%10ccccc%10c9)cc8)cc7c6o5)cc4)cc3o2)cc1 ; c1ccc(-c2nc3ccc(-c4ccc(-c5nc6c7ccc(-c8ccc(-c9ccc%10ccccc%10c9)cc8)cc7c7cc(-c8ccc(-c9ccc%10ccccc%10c9)cc8)ccc7c6o5)cc4)cc3o2)cc1]

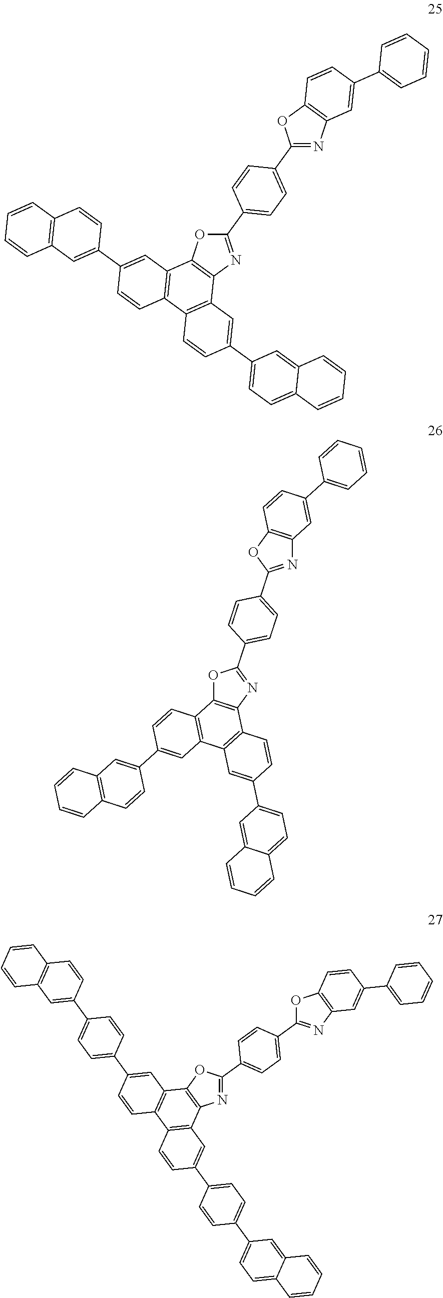 [SMILES: c1ccc(-c2ccc3oc(-c4ccc(-c5nc6c7cc(-c8ccc9ccccc9c8)ccc7c7ccc(-c8ccc9ccccc9c8)cc7c6o5)cc4)nc3c2)cc1 ; c1ccc(-c2ccc3oc(-c4ccc(-c5nc6c7ccc(-c8ccc9ccccc9c8)cc7c7cc(-c8ccc9ccccc9c8)ccc7c6o5)cc4)nc3c2)cc1 ; c1ccc(-c2ccc3oc(-c4ccc(-c5nc6c7cc(-c8ccc(-c9ccc%10ccccc%10c9)cc8)ccc7c7ccc(-c8ccc(-c9ccc%10ccccc%10c9)cc8)cc7c6o5)cc4)nc3c2)cc1]

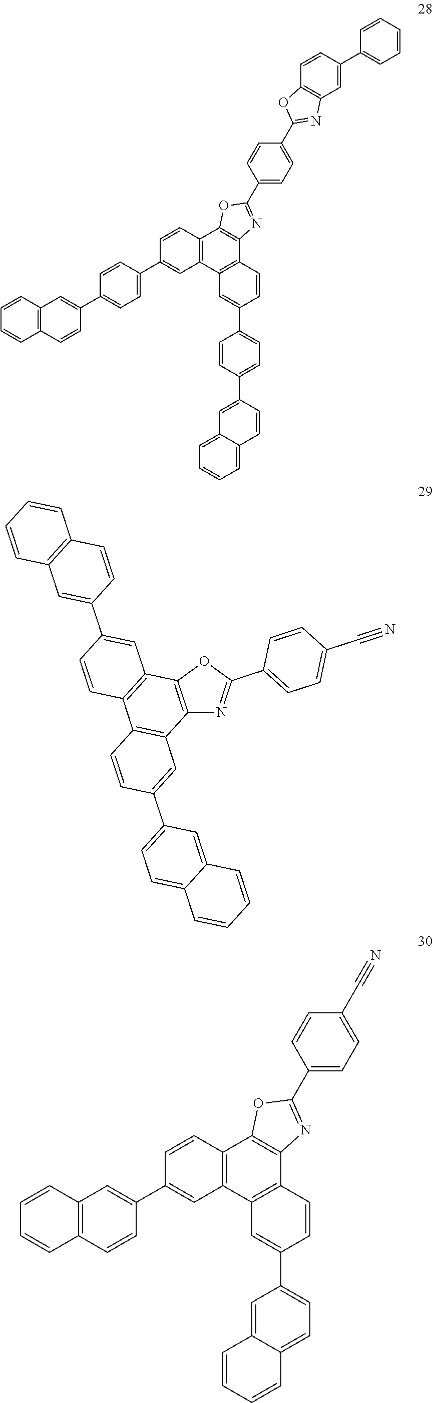 [SMILES: c1ccc(-c2ccc3oc(-c4ccc(-c5nc6c7ccc(-c8ccc(-c9ccc%10ccccc%10c9)cc8)cc7c7cc(-c8ccc(-c9ccc%10ccccc%10c9)cc8)ccc7c6o5)cc4)nc3c2)cc1 ; N#Cc1ccc(-c2nc3c4cc(-c5ccc6ccccc6c5)ccc4c4ccc(-c5ccc6ccccc6c5)cc4c3o2)cc1 ; N#Cc1ccc(-c2nc3c4ccc(-c5ccc6ccccc6c5)cc4c4cc(-c5ccc6ccccc6c5)ccc4c3o2)cc1]

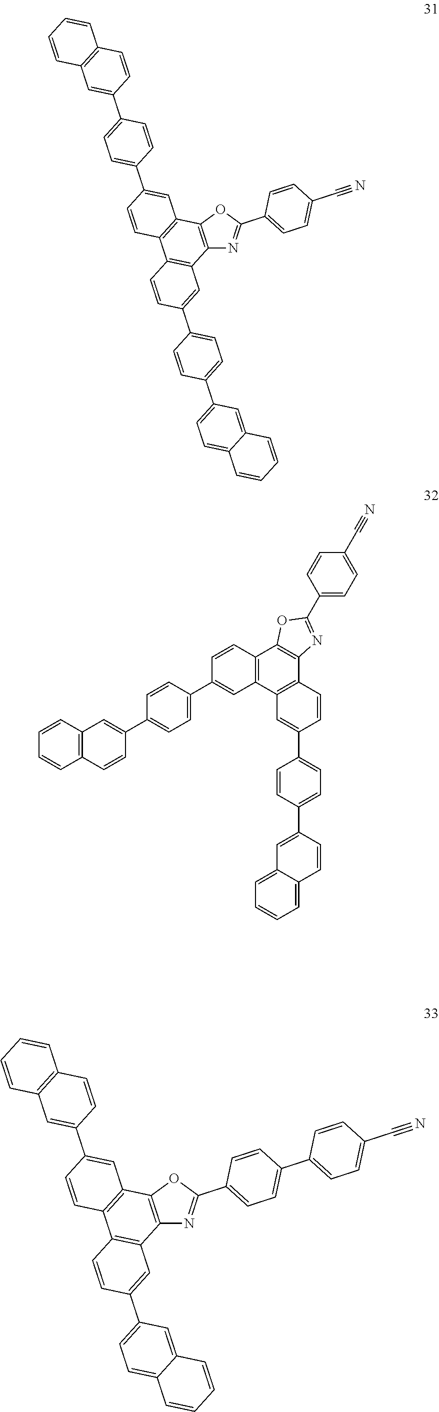 [SMILES: N#Cc1ccc(-c2nc3c4cc(-c5ccc(-c6ccc7ccccc7c6)cc5)ccc4c4ccc(-c5ccc(-c6ccc7ccccc7c6)cc5)cc4c3o2)cc1 ; N#Cc1ccc(-c2nc3c4ccc(-c5ccc(-c6ccc7ccccc7c6)cc5)cc4c4cc(-c5ccc(-c6ccc7ccccc7c6)cc5)ccc4c3o2)cc1 ; N#Cc1ccc(-c2ccc(-c3nc4c5cc(-c6ccc7ccccc7c6)ccc5c5ccc(-c6ccc7ccccc7c6)cc5c4o3)cc2)cc1]

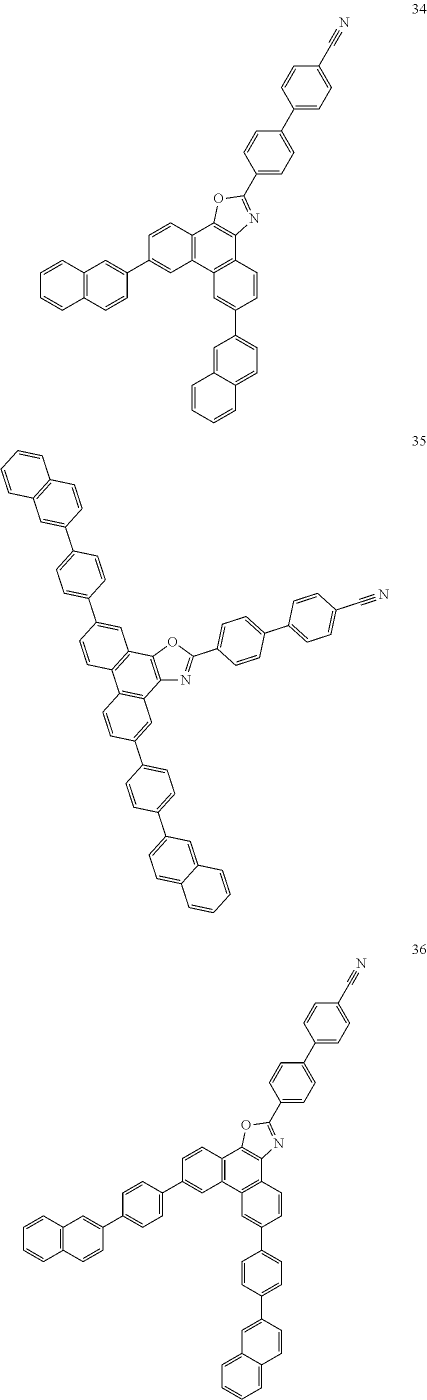 [SMILES: N#Cc1ccc(-c2ccc(-c3nc4c5ccc(-c6ccc7ccccc7c6)cc5c5cc(-c6ccc7ccccc7c6)ccc5c4o3)cc2)cc1 ; N#Cc1ccc(-c2ccc(-c3nc4c5cc(-c6ccc(-c7ccc8ccccc8c7)cc6)ccc5c5ccc(-c6ccc(-c7ccc8ccccc8c7)cc6)cc5c4o3)cc2)cc1 ; N#Cc1ccc(-c2ccc(-c3nc4c5ccc(-c6ccc(-c7ccc8ccccc8c7)cc6)cc5c5cc(-c6ccc(-c7ccc8ccccc8c7)cc6)ccc5c4o3)cc2)cc1]

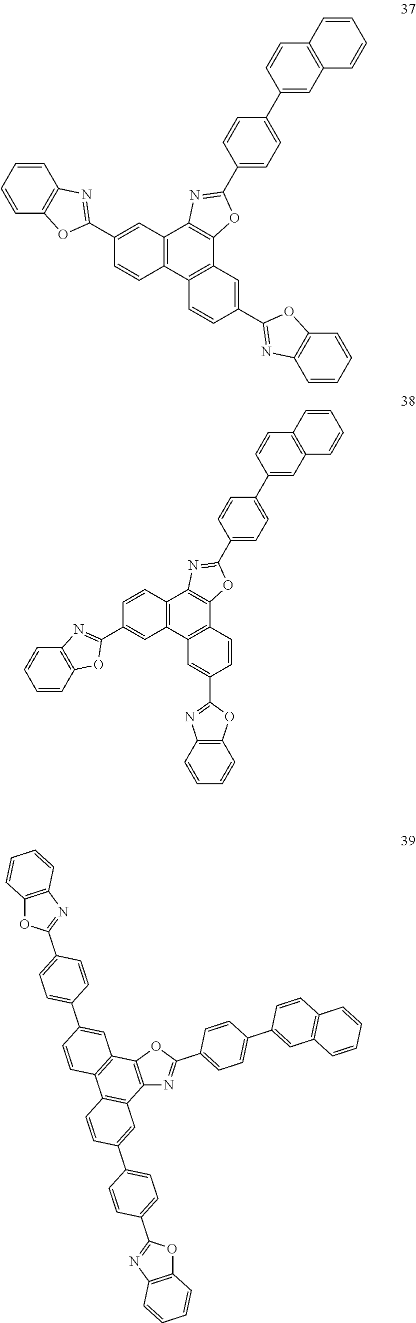

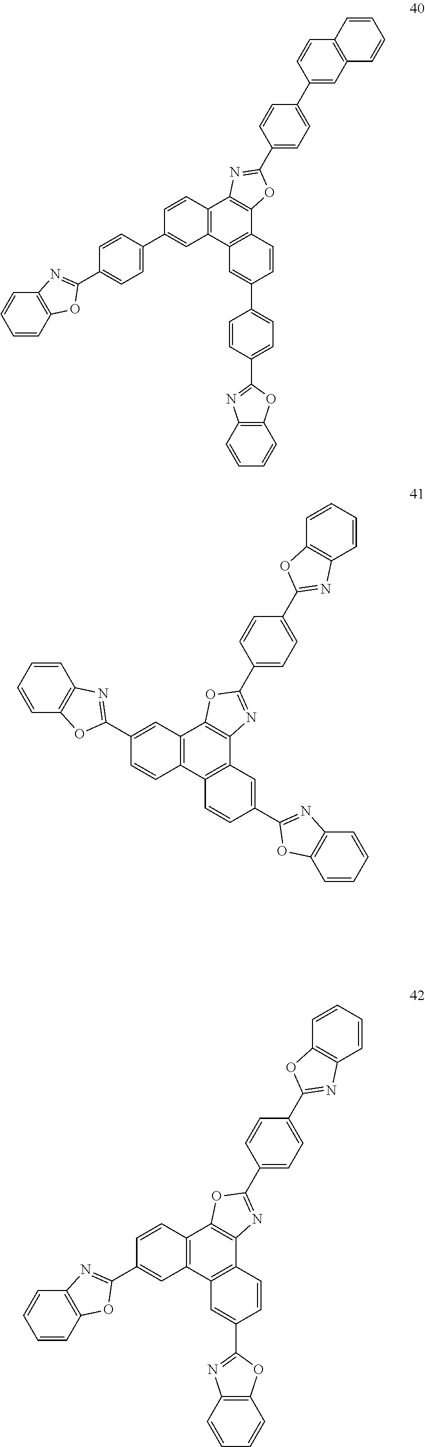 [SMILES: c1ccc2cc(-c3ccc(-c4nc5c6ccc(-c7ccc(-c8nc9ccccc9o8)cc7)cc6c6cc(-c7ccc(-c8nc9ccccc9o8)cc7)ccc6c5o4)cc3)ccc2c1 ; c1ccc2oc(-c3ccc(-c4nc5c6cc(-c7nc8ccccc8o7)ccc6c6ccc(-c7nc8ccccc8o7)cc6c5o4)cc3)nc2c1 ; c1ccc2oc(-c3ccc(-c4nc5c6ccc(-c7nc8ccccc8o7)cc6c6cc(-c7nc8ccccc8o7)ccc6c5o4)cc3)nc2c1]

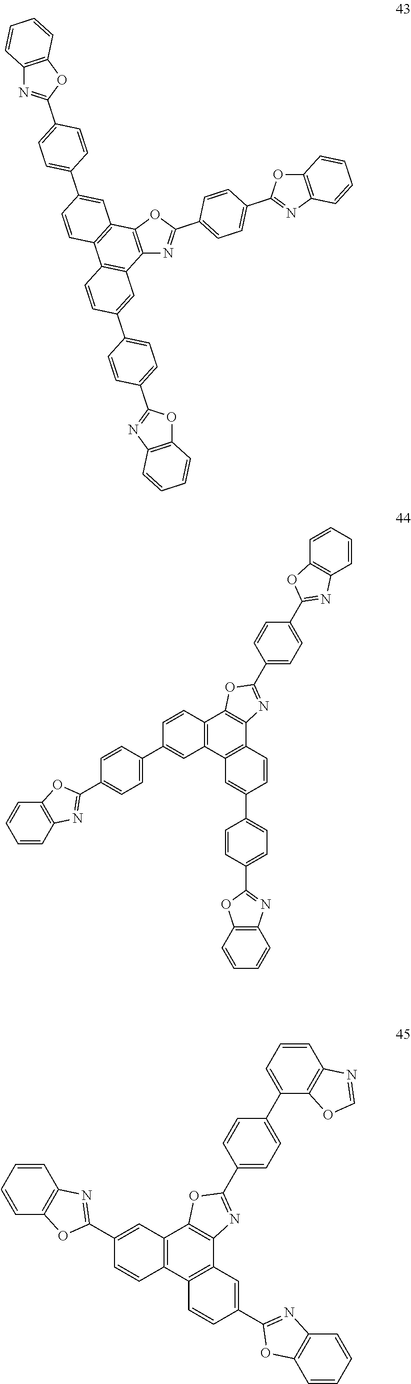 [SMILES: c1ccc2oc(-c3ccc(-c4ccc5c6ccc(-c7ccc(-c8nc9ccccc9o8)cc7)cc6c6oc(-c7ccc(-c8nc9ccccc9o8)cc7)nc6c5c4)cc3)nc2c1 ; c1ccc2oc(-c3ccc(-c4ccc5c(c4)c4cc(-c6ccc(-c7nc8ccccc8o7)cc6)ccc4c4oc(-c6ccc(-c7nc8ccccc8o7)cc6)nc54)cc3)nc2c1 ; c1ccc2oc(-c3ccc4c5ccc(-c6nc7ccccc7o6)cc5c5oc(-c6ccc(-c7cccc8ncoc78)cc6)nc5c4c3)nc2c1]

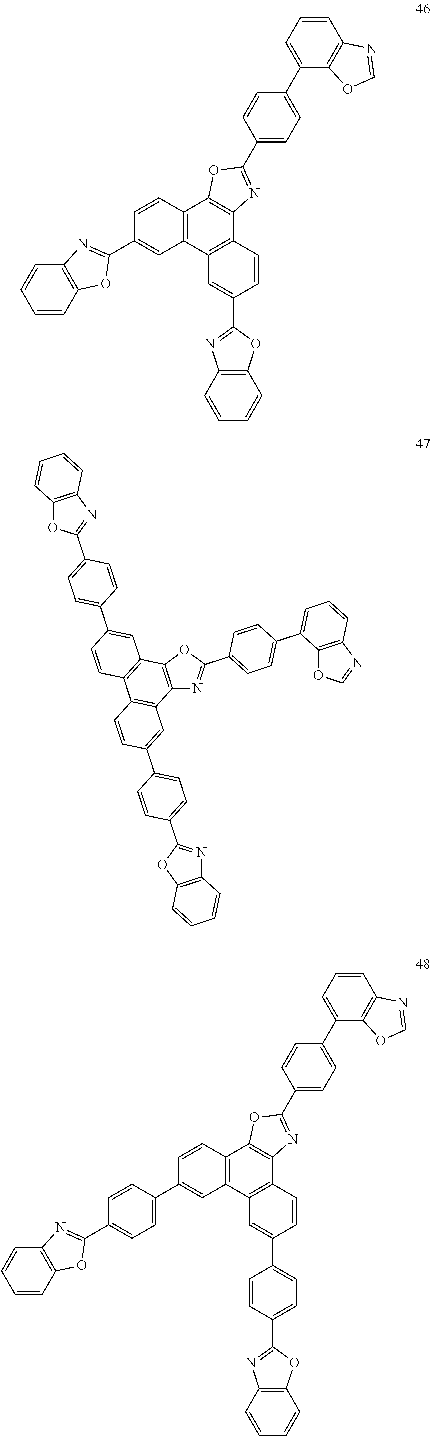 [SMILES: c1ccc2oc(-c3ccc4c(c3)c3cc(-c5nc6ccccc6o5)ccc3c3oc(-c5ccc(-c6cccc7ncoc67)cc5)nc43)nc2c1 ; c1ccc2oc(-c3ccc(-c4ccc5c6ccc(-c7ccc(-c8nc9ccccc9o8)cc7)cc6c6oc(-c7ccc(-c8cccc9ncoc89)cc7)nc6c5c4)cc3)nc2c1 ; c1ccc2oc(-c3ccc(-c4ccc5c(c4)c4cc(-c6ccc(-c7nc8ccccc8o7)cc6)ccc4c4oc(-c6ccc(-c7cccc8ncoc78)cc6)nc54)cc3)nc2c1]

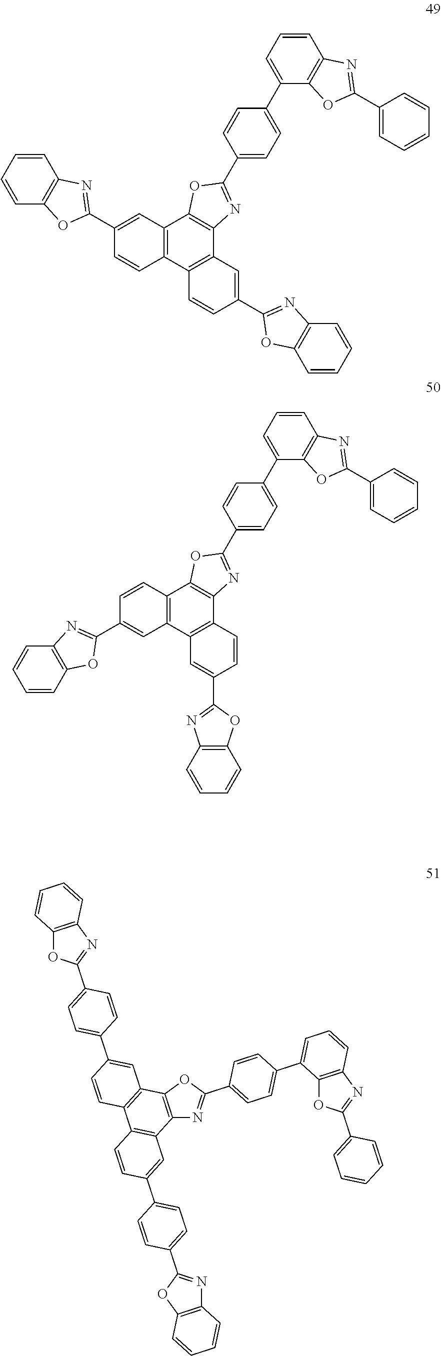 [SMILES: c1ccc(-c2nc3cccc(-c4ccc(-c5nc6c7cc(-c8nc9ccccc9o8)ccc7c7ccc(-c8nc9ccccc9o8)cc7c6o5)cc4)c3o2)cc1 ; c1ccc(-c2nc3cccc(-c4ccc(-c5nc6c7ccc(-c8nc9ccccc9o8)cc7c7cc(-c8nc9ccccc9o8)ccc7c6o5)cc4)c3o2)cc1 ; c1ccc(-c2nc3cccc(-c4ccc(-c5nc6c7cc(-c8ccc(-c9nc%10ccccc%10o9)cc8)ccc7c7ccc(-c8ccc(-c9nc%10ccccc%10o9)cc8)cc7c6o5)cc4)c3o2)cc1]

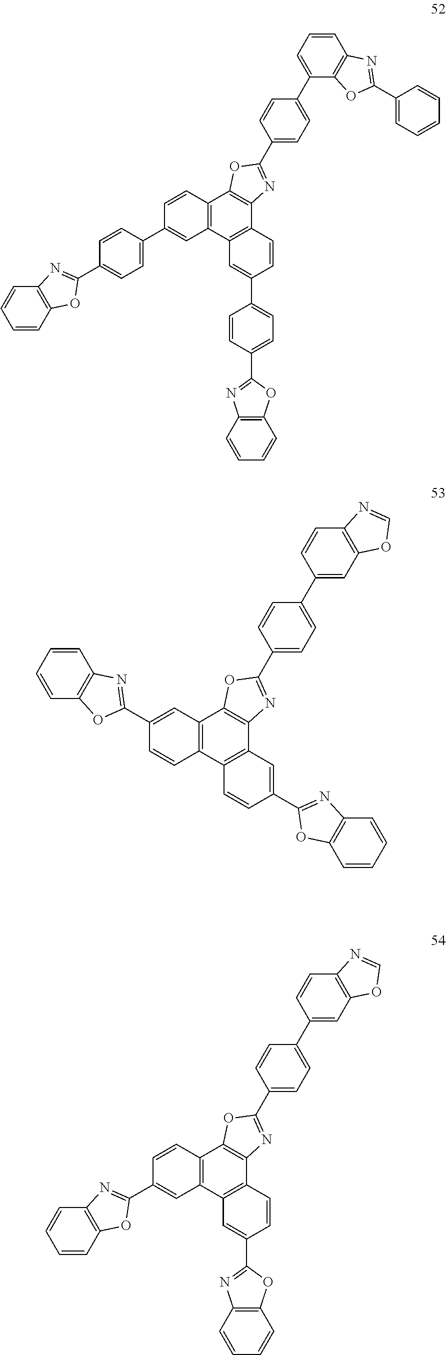 [SMILES: c1ccc(-c2nc3cccc(-c4ccc(-c5nc6c7ccc(-c8ccc(-c9nc%10ccccc%10o9)cc8)cc7c7cc(-c8ccc(-c9nc%10ccccc%10o9)cc8)ccc7c6o5)cc4)c3o2)cc1 ; c1ccc2oc(-c3ccc4c5ccc(-c6nc7ccccc7o6)cc5c5oc(-c6ccc(-c7ccc8ncoc8c7)cc6)nc5c4c3)nc2c1 ; c1ccc2oc(-c3ccc4c(c3)c3cc(-c5nc6ccccc6o5)ccc3c3oc(-c5ccc(-c6ccc7ncoc7c6)cc5)nc43)nc2c1]

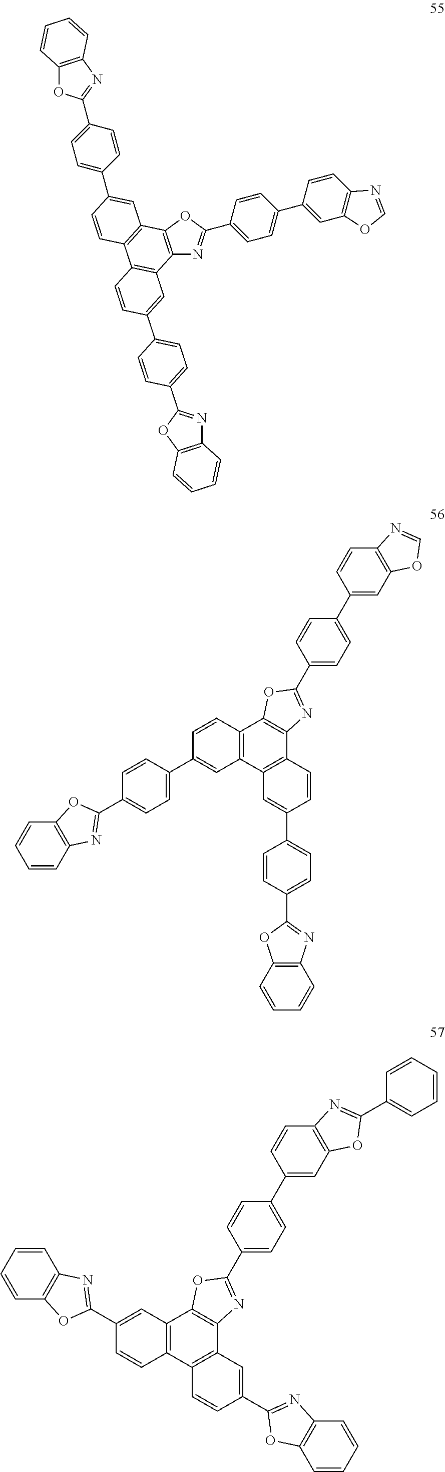 [SMILES: c1ccc2oc(-c3ccc(-c4ccc5c6ccc(-c7ccc(-c8nc9ccccc9o8)cc7)cc6c6oc(-c7ccc(-c8ccc9ncoc9c8)cc7)nc6c5c4)cc3)nc2c1 ; c1ccc2oc(-c3ccc(-c4ccc5c(c4)c4cc(-c6ccc(-c7nc8ccccc8o7)cc6)ccc4c4oc(-c6ccc(-c7ccc8ncoc8c7)cc6)nc54)cc3)nc2c1 ; c1ccc(-c2nc3ccc(-c4ccc(-c5nc6c7cc(-c8nc9ccccc9o8)ccc7c7ccc(-c8nc9ccccc9o8)cc7c6o5)cc4)cc3o2)cc1]

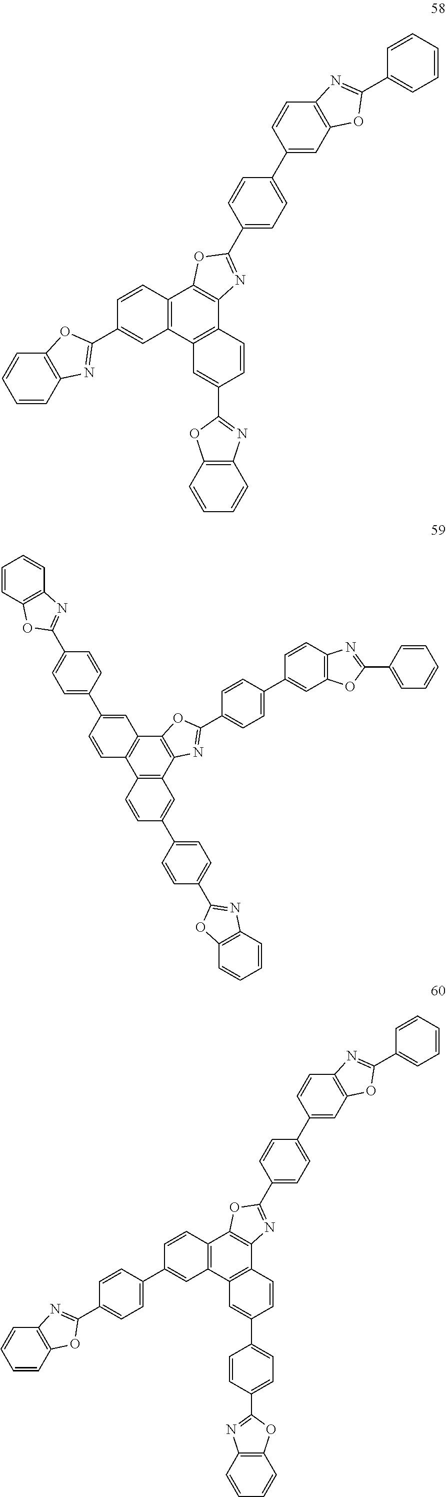 [SMILES: c1ccc(-c2nc3ccc(-c4ccc(-c5nc6c7ccc(-c8nc9ccccc9o8)cc7c7cc(-c8nc9ccccc9o8)ccc7c6o5)cc4)cc3o2)cc1 ; c1ccc(-c2nc3ccc(-c4ccc(-c5nc6c7cc(-c8ccc(-c9nc%10ccccc%10o9)cc8)ccc7c7ccc(-c8ccc(-c9nc%10ccccc%10o9)cc8)cc7c6o5)cc4)cc3o2)cc1 ; c1ccc(-c2nc3ccc(-c4ccc(-c5nc6c7ccc(-c8ccc(-c9nc%10ccccc%10o9)cc8)cc7c7cc(-c8ccc(-c9nc%10ccccc%10o9)cc8)ccc7c6o5)cc4)cc3o2)cc1]

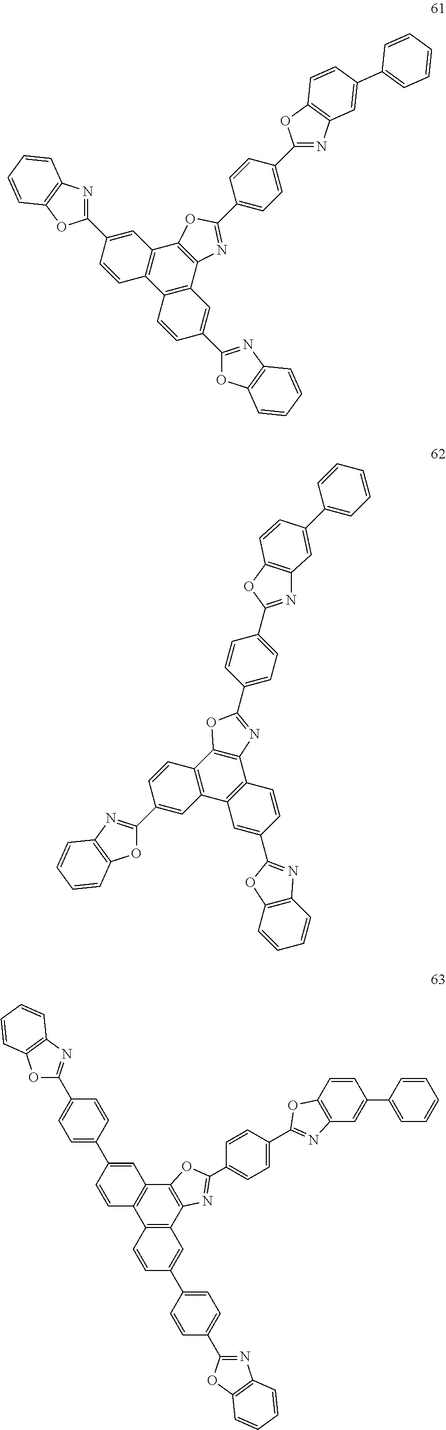 [SMILES: c1ccc(-c2ccc3oc(-c4ccc(-c5nc6c7cc(-c8nc9ccccc9o8)ccc7c7ccc(-c8nc9ccccc9o8)cc7c6o5)cc4)nc3c2)cc1 ; c1ccc(-c2ccc3oc(-c4ccc(-c5nc6c7ccc(-c8nc9ccccc9o8)cc7c7cc(-c8nc9ccccc9o8)ccc7c6o5)cc4)nc3c2)cc1 ; c1ccc(-c2ccc3oc(-c4ccc(-c5nc6c7cc(-c8ccc(-c9nc%10ccccc%10o9)cc8)ccc7c7ccc(-c8ccc(-c9nc%10ccccc%10o9)cc8)cc7c6o5)cc4)nc3c2)cc1]

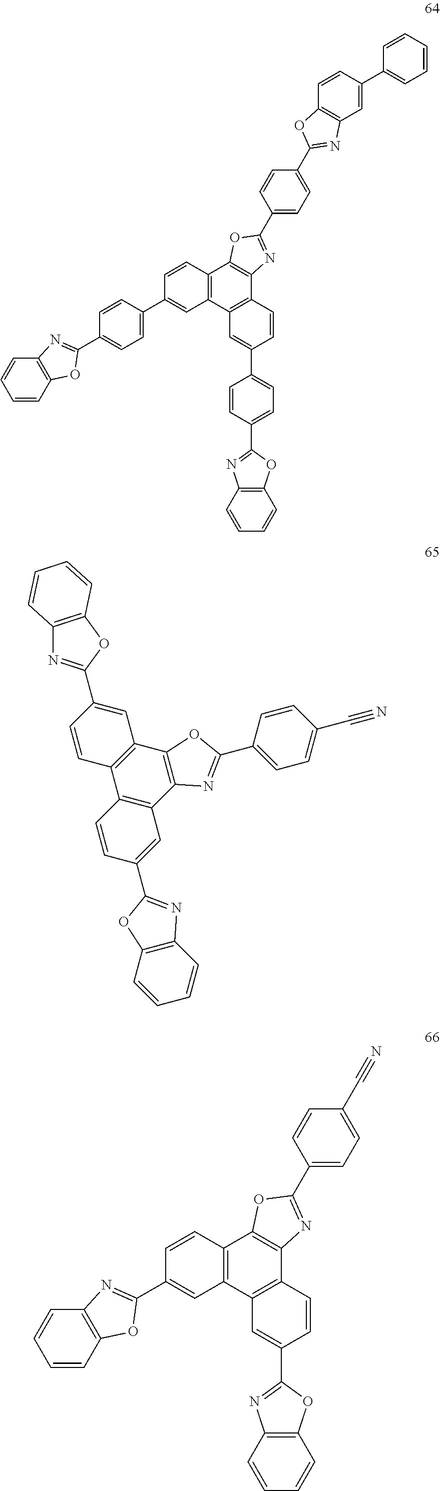 [SMILES: c1ccc(-c2ccc3oc(-c4ccc(-c5nc6c7ccc(-c8ccc(-c9nc%10ccccc%10o9)cc8)cc7c7cc(-c8ccc(-c9nc%10ccccc%10o9)cc8)ccc7c6o5)cc4)nc3c2)cc1 ; N#Cc1ccc(-c2nc3c4cc(-c5nc6ccccc6o5)ccc4c4ccc(-c5nc6ccccc6o5)cc4c3o2)cc1 ; N#Cc1ccc(-c2nc3c4ccc(-c5nc6ccccc6o5)cc4c4cc(-c5nc6ccccc6o5)ccc4c3o2)cc1]

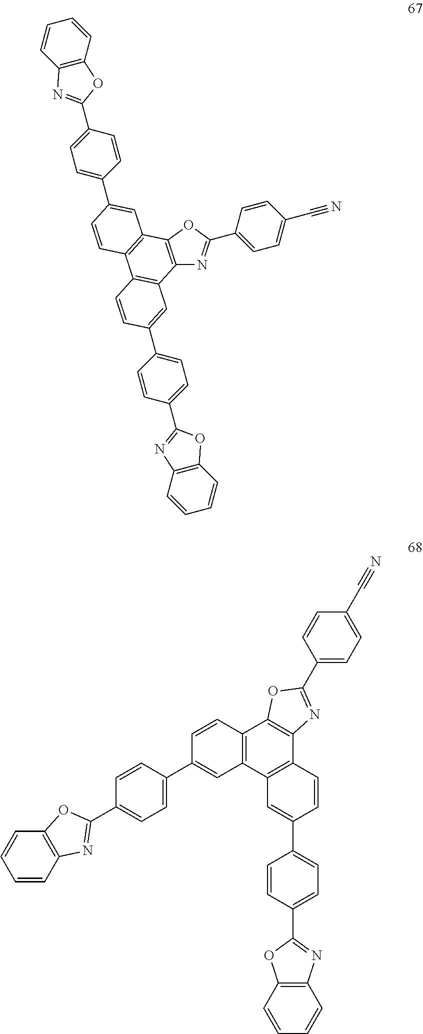 [SMILES: N#Cc1ccc(-c2nc3c4cc(-c5ccc(-c6nc7ccccc7o6)cc5)ccc4c4ccc(-c5ccc(-c6nc7ccccc7o6)cc5)cc4c3o2)cc1 ; N#Cc1ccc(-c2nc3c4ccc(-c5ccc(-c6nc7ccccc7o6)cc5)cc4c4cc(-c5ccc(-c6nc7ccccc7o6)cc5)ccc4c3o2)cc1]

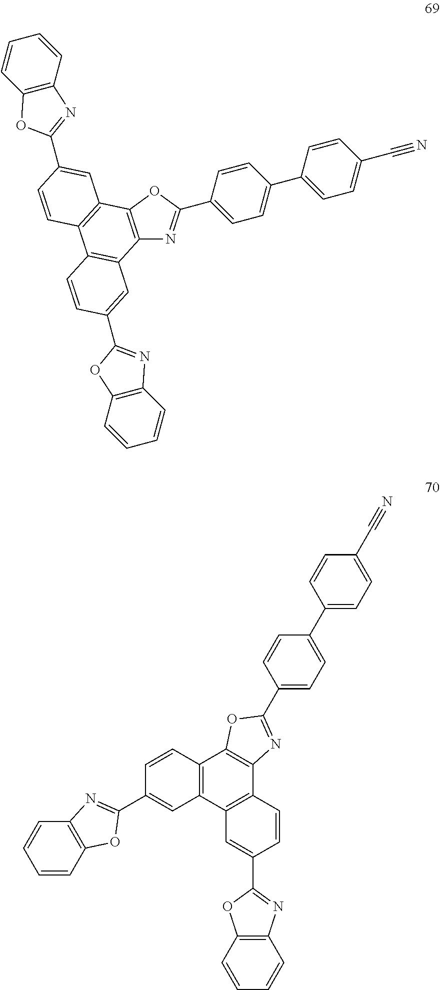 [SMILES: N#Cc1ccc(-c2ccc(-c3nc4c5cc(-c6nc7ccccc7o6)ccc5c5ccc(-c6nc7ccccc7o6)cc5c4o3)cc2)cc1 ; N#Cc1ccc(-c2ccc(-c3nc4c5ccc(-c6nc7ccccc7o6)cc5c5cc(-c6nc7ccccc7o6)ccc5c4o3)cc2)cc1]

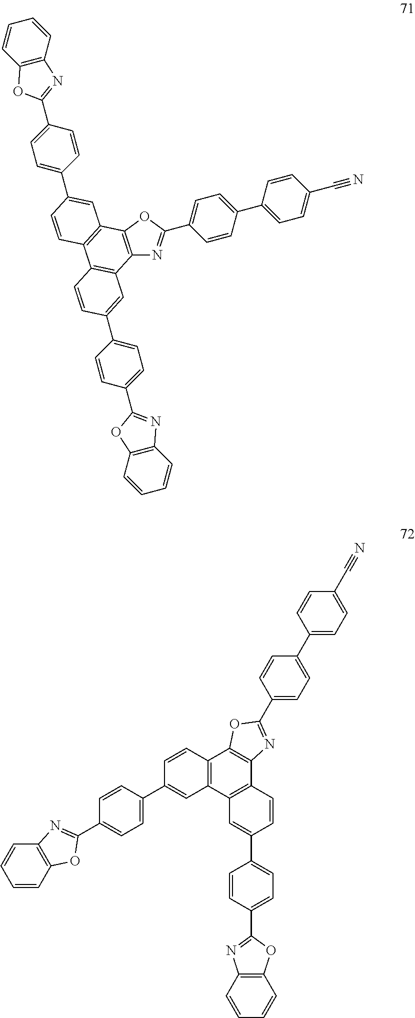 [SMILES: N#Cc1ccc(-c2ccc(-c3nc4c5cc(-c6ccc(-c7nc8ccccc8o7)cc6)ccc5c5ccc(-c6ccc(-c7nc8ccccc8o7)cc6)cc5c4o3)cc2)cc1 ; N#Cc1ccc(-c2ccc(-c3nc4c5ccc(-c6ccc(-c7nc8ccccc8o7)cc6)cc5c5cc(-c6ccc(-c7nc8ccccc8o7)cc6)ccc5c4o3)cc2)cc1]

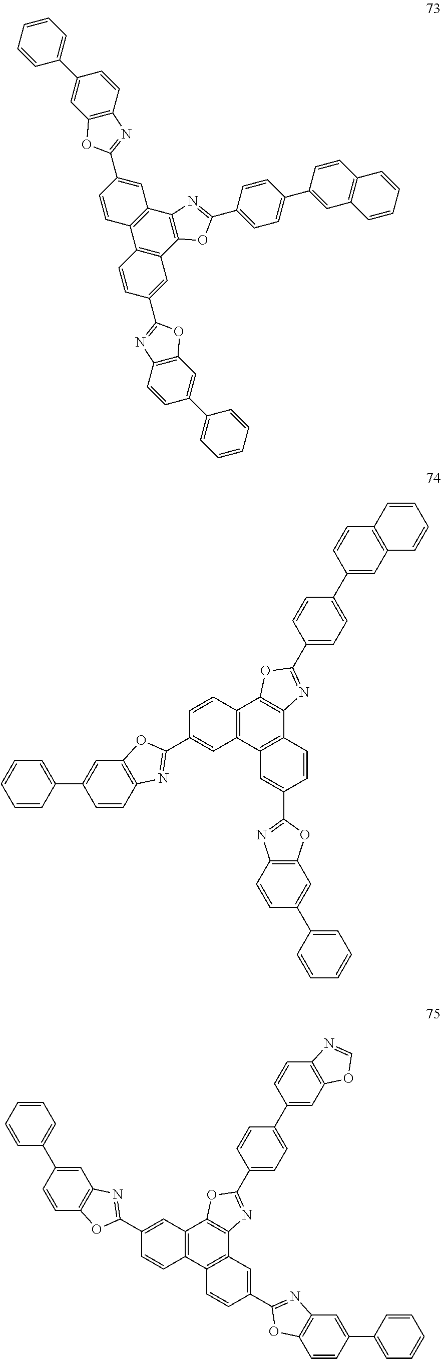 [SMILES: c1ccc(-c2ccc3nc(-c4ccc5c6ccc(-c7nc8ccc(-c9ccccc9)cc8o7)cc6c6oc(-c7ccc(-c8ccc9ccccc9c8)cc7)nc6c5c4)oc3c2)cc1 ; c1ccc(-c2ccc3nc(-c4ccc5c(c4)c4cc(-c6nc7ccc(-c8ccccc8)cc7o6)ccc4c4oc(-c6ccc(-c7ccc8ccccc8c7)cc6)nc54)oc3c2)cc1 ; c1ccc(-c2ccc3oc(-c4ccc5c6ccc(-c7nc8cc(-c9ccccc9)ccc8o7)cc6c6oc(-c7ccc(-c8ccc9ncoc9c8)cc7)nc6c5c4)nc3c2)cc1]

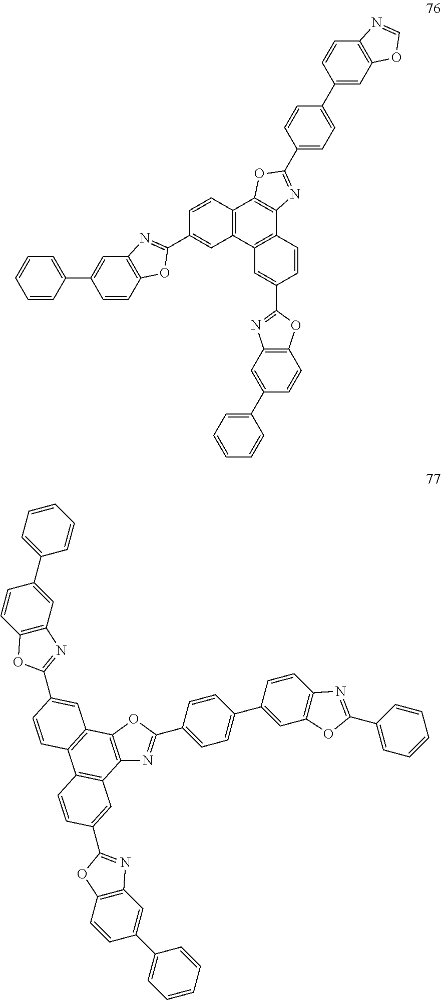 [SMILES: c1ccc(-c2ccc3oc(-c4ccc5c(c4)c4cc(-c6nc7cc(-c8ccccc8)ccc7o6)ccc4c4oc(-c6ccc(-c7ccc8ncoc8c7)cc6)nc54)nc3c2)cc1 ; c1ccc(-c2ccc3oc(-c4ccc5c6ccc(-c7nc8cc(-c9ccccc9)ccc8o7)cc6c6oc(-c7ccc(-c8ccc9nc(-c%10ccccc%10)oc9c8)cc7)nc6c5c4)nc3c2)cc1]

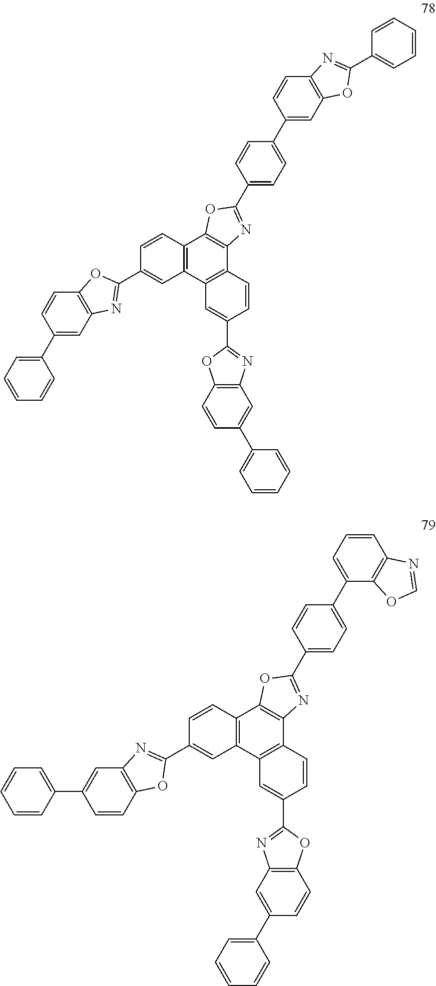 [SMILES: c1ccc(-c2ccc3oc(-c4ccc5c(c4)c4cc(-c6nc7cc(-c8ccccc8)ccc7o6)ccc4c4oc(-c6ccc(-c7ccc8nc(-c9ccccc9)oc8c7)cc6)nc54)nc3c2)cc1 ; c1ccc(-c2ccc3oc(-c4ccc5c(c4)c4cc(-c6nc7cc(-c8ccccc8)ccc7o6)ccc4c4oc(-c6ccc(-c7cccc8ncoc78)cc6)nc54)nc3c2)cc1]

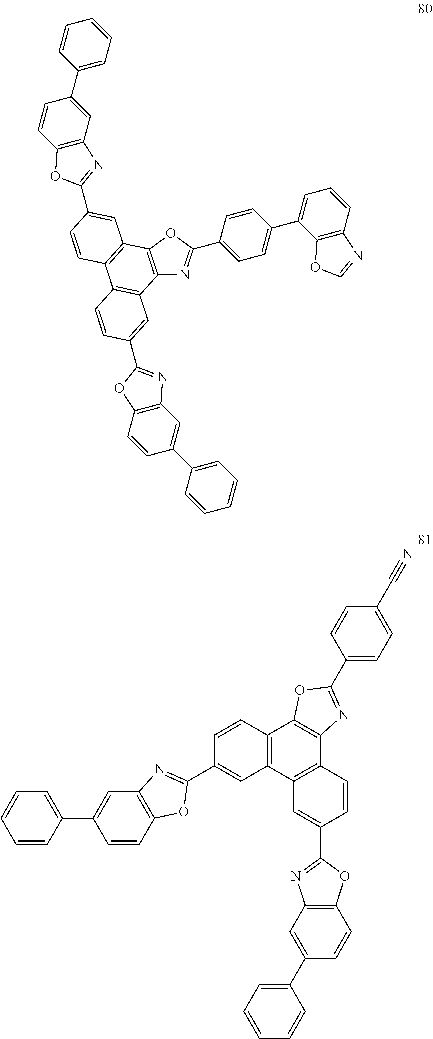 [SMILES: c1ccc(-c2ccc3oc(-c4ccc5c6ccc(-c7nc8cc(-c9ccccc9)ccc8o7)cc6c6oc(-c7ccc(-c8cccc9ncoc89)cc7)nc6c5c4)nc3c2)cc1 ; N#Cc1ccc(-c2nc3c4ccc(-c5nc6cc(-c7ccccc7)ccc6o5)cc4c4cc(-c5nc6cc(-c7ccccc7)ccc6o5)ccc4c3o2)cc1]

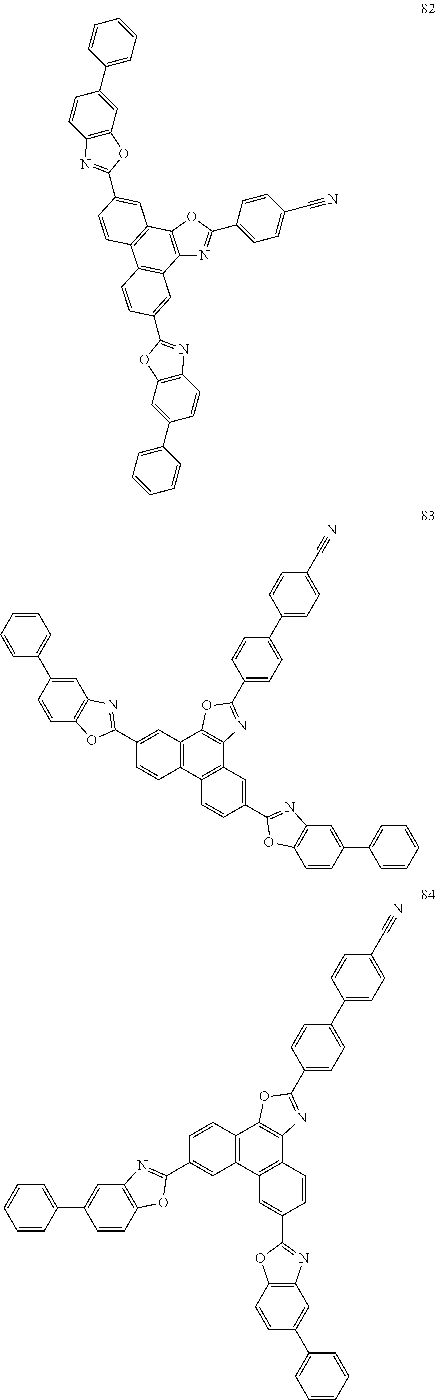 [SMILES: N#Cc1ccc(-c2nc3c4cc(-c5nc6ccc(-c7ccccc7)cc6o5)ccc4c4ccc(-c5nc6ccc(-c7ccccc7)cc6o5)cc4c3o2)cc1 ; N#Cc1ccc(-c2ccc(-c3nc4c5cc(-c6nc7cc(-c8ccccc8)ccc7o6)ccc5c5ccc(-c6nc7cc(-c8ccccc8)ccc7o6)cc5c4o3)cc2)cc1 ; N#Cc1ccc(-c2ccc(-c3nc4c5ccc(-c6nc7cc(-c8ccccc8)ccc7o6)cc5c5cc(-c6nc7cc(-c8ccccc8)ccc7o6)ccc5c4o3)cc2)cc1]

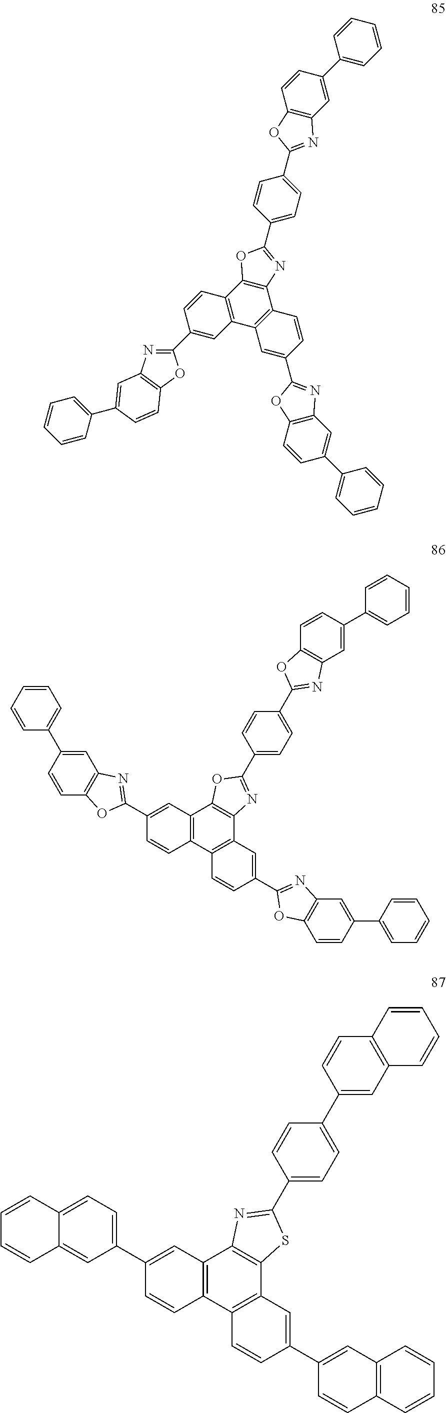 [SMILES: c1ccc(-c2ccc3oc(-c4ccc(-c5nc6c7ccc(-c8nc9cc(-c%10ccccc%10)ccc9o8)cc7c7cc(-c8nc9cc(-c%10ccccc%10)ccc9o8)ccc7c6o5)cc4)nc3c2)cc1 ; c1ccc(-c2ccc3oc(-c4ccc(-c5nc6c7cc(-c8nc9cc(-c%10ccccc%10)ccc9o8)ccc7c7ccc(-c8nc9cc(-c%10ccccc%10)ccc9o8)cc7c6o5)cc4)nc3c2)cc1 ; c1ccc2cc(-c3ccc(-c4nc5c6cc(-c7ccc8ccccc8c7)ccc6c6ccc(-c7ccc8ccccc8c7)cc6c5s4)cc3)ccc2c1]

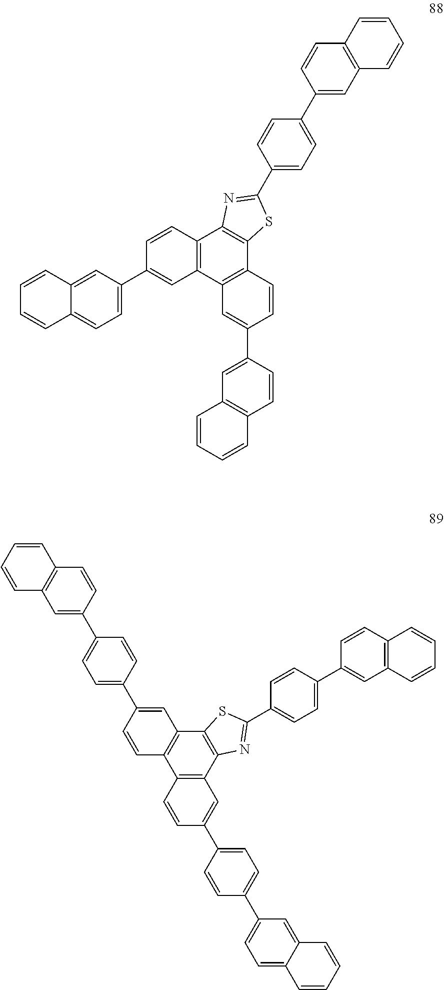 [SMILES: c1ccc2cc(-c3ccc(-c4nc5c6ccc(-c7ccc8ccccc8c7)cc6c6cc(-c7ccc8ccccc8c7)ccc6c5s4)cc3)ccc2c1 ; c1ccc2cc(-c3ccc(-c4ccc5c6ccc(-c7ccc(-c8ccc9ccccc9c8)cc7)cc6c6sc(-c7ccc(-c8ccc9ccccc9c8)cc7)nc6c5c4)cc3)ccc2c1]

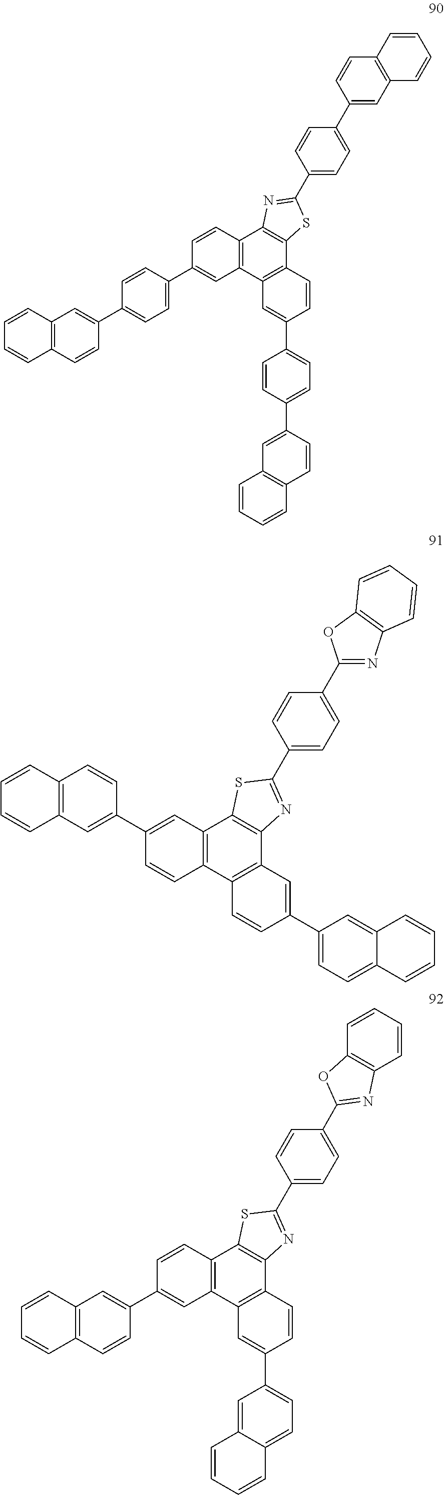

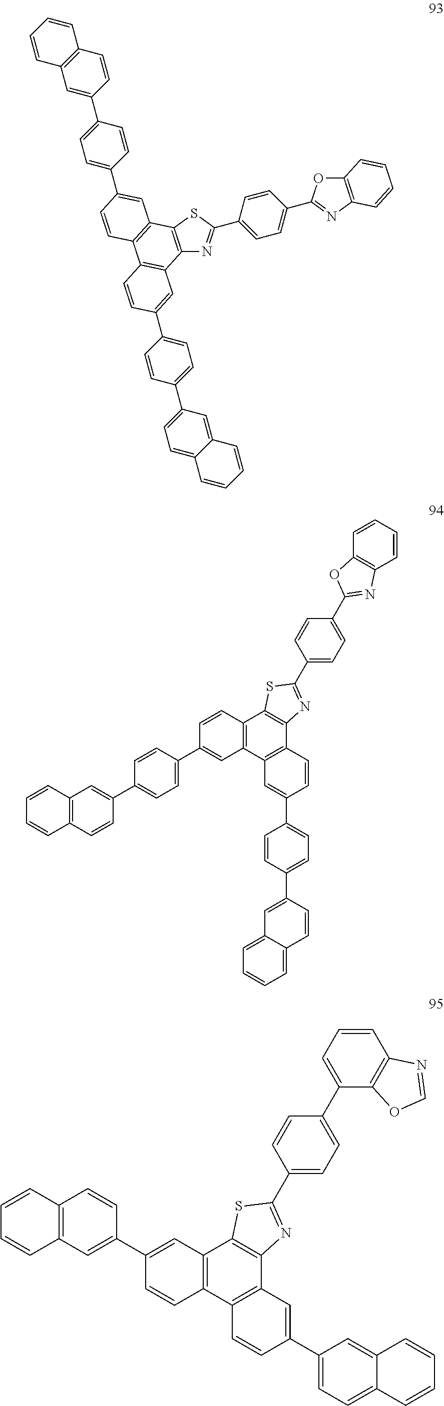 [SMILES: c1ccc2cc(-c3ccc(-c4ccc5c6ccc(-c7ccc(-c8ccc9ccccc9c8)cc7)cc6c6sc(-c7ccc(-c8nc9ccccc9o8)cc7)nc6c5c4)cc3)ccc2c1 ; c1ccc2cc(-c3ccc(-c4ccc5c(c4)c4cc(-c6ccc(-c7ccc8ccccc8c7)cc6)ccc4c4sc(-c6ccc(-c7nc8ccccc8o7)cc6)nc54)cc3)ccc2c1 ; c1ccc2cc(-c3ccc4c5ccc(-c6ccc7ccccc7c6)cc5c5sc(-c6ccc(-c7cccc8ncoc78)cc6)nc5c4c3)ccc2c1]

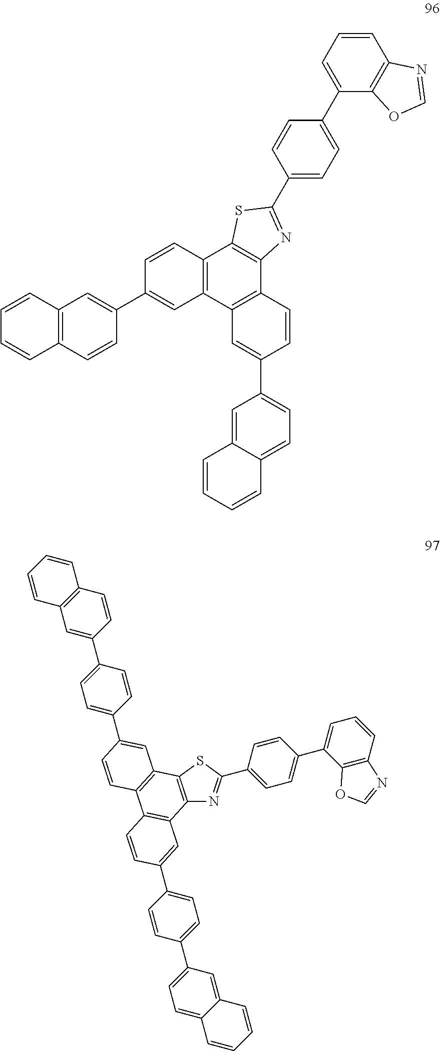 [SMILES: c1ccc2cc(-c3ccc4c(c3)c3cc(-c5ccc6ccccc6c5)ccc3c3sc(-c5ccc(-c6cccc7ncoc67)cc5)nc43)ccc2c1 ; c1ccc2cc(-c3ccc(-c4ccc5c6ccc(-c7ccc(-c8ccc9ccccc9c8)cc7)cc6c6sc(-c7ccc(-c8cccc9ncoc89)cc7)nc6c5c4)cc3)ccc2c1]

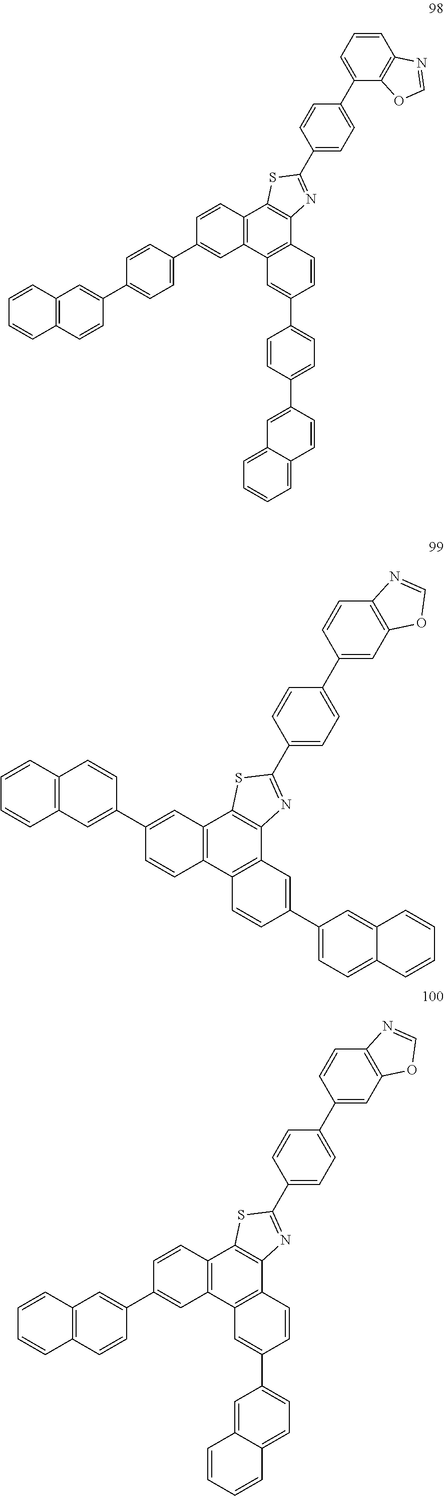 [SMILES: c1ccc2cc(-c3ccc(-c4ccc5c(c4)c4cc(-c6ccc(-c7ccc8ccccc8c7)cc6)ccc4c4sc(-c6ccc(-c7cccc8ncoc78)cc6)nc54)cc3)ccc2c1 ; c1ccc2cc(-c3ccc4c5ccc(-c6ccc7ccccc7c6)cc5c5sc(-c6ccc(-c7ccc8ncoc8c7)cc6)nc5c4c3)ccc2c1 ; c1ccc2cc(-c3ccc4c(c3)c3cc(-c5ccc6ccccc6c5)ccc3c3sc(-c5ccc(-c6ccc7ncoc7c6)cc5)nc43)ccc2c1]

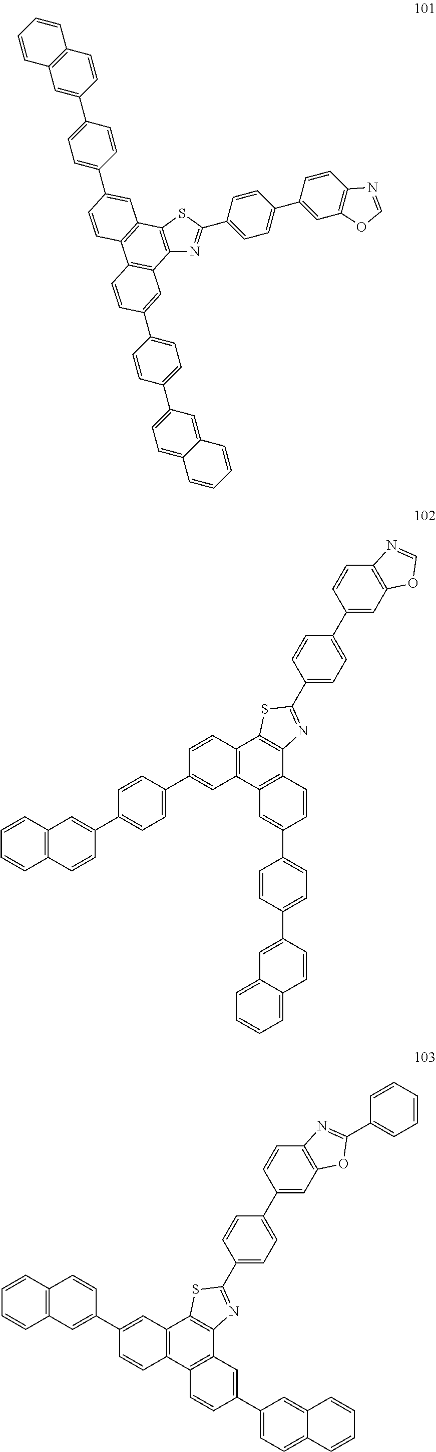 [SMILES: c1ccc2cc(-c3ccc(-c4ccc5c6ccc(-c7ccc(-c8ccc9ccccc9c8)cc7)cc6c6sc(-c7ccc(-c8ccc9ncoc9c8)cc7)nc6c5c4)cc3)ccc2c1 ; c1ccc2cc(-c3ccc(-c4ccc5c(c4)c4cc(-c6ccc(-c7ccc8ccccc8c7)cc6)ccc4c4sc(-c6ccc(-c7ccc8ncoc8c7)cc6)nc54)cc3)ccc2c1 ; c1ccc(-c2nc3ccc(-c4ccc(-c5nc6c7cc(-c8ccc9ccccc9c8)ccc7c7ccc(-c8ccc9ccccc9c8)cc7c6s5)cc4)cc3o2)cc1]

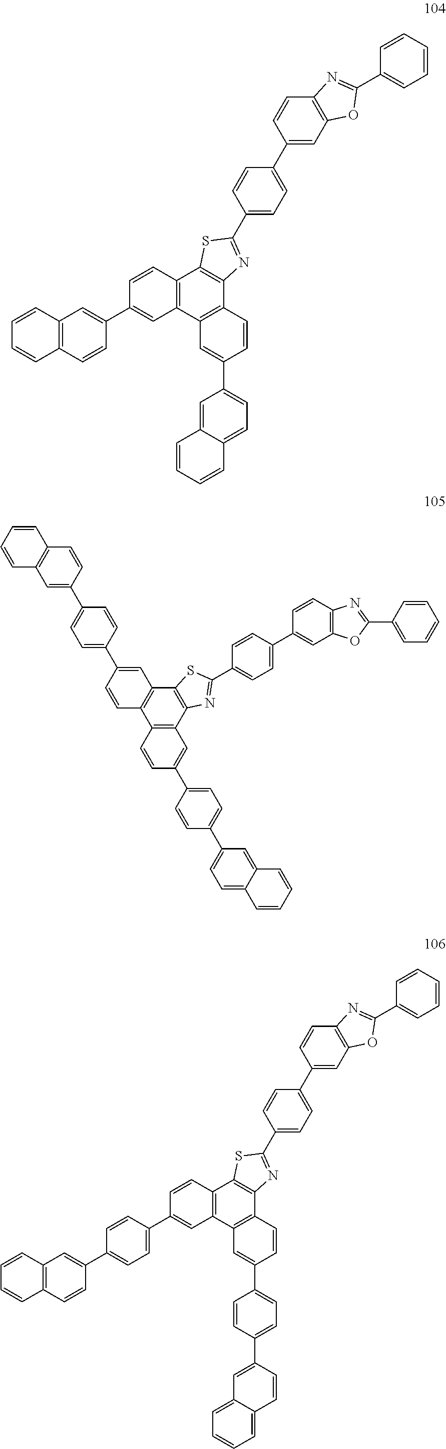 [SMILES: c1ccc(-c2nc3ccc(-c4ccc(-c5nc6c7ccc(-c8ccc9ccccc9c8)cc7c7cc(-c8ccc9ccccc9c8)ccc7c6s5)cc4)cc3o2)cc1 ; c1ccc(-c2nc3ccc(-c4ccc(-c5nc6c7cc(-c8ccc(-c9ccc%10ccccc%10c9)cc8)ccc7c7ccc(-c8ccc(-c9ccc%10ccccc%10c9)cc8)cc7c6s5)cc4)cc3o2)cc1 ; c1ccc(-c2nc3ccc(-c4ccc(-c5nc6c7ccc(-c8ccc(-c9ccc%10ccccc%10c9)cc8)cc7c7cc(-c8ccc(-c9ccc%10ccccc%10c9)cc8)ccc7c6s5)cc4)cc3o2)cc1]

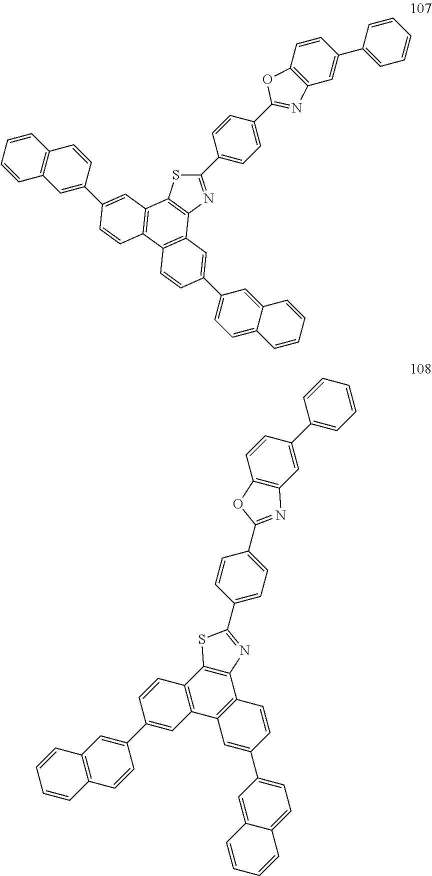 [SMILES: c1ccc(-c2ccc3oc(-c4ccc(-c5nc6c7cc(-c8ccc9ccccc9c8)ccc7c7ccc(-c8ccc9ccccc9c8)cc7c6s5)cc4)nc3c2)cc1 ; c1ccc(-c2ccc3oc(-c4ccc(-c5nc6c7ccc(-c8ccc9ccccc9c8)cc7c7cc(-c8ccc9ccccc9c8)ccc7c6s5)cc4)nc3c2)cc1]

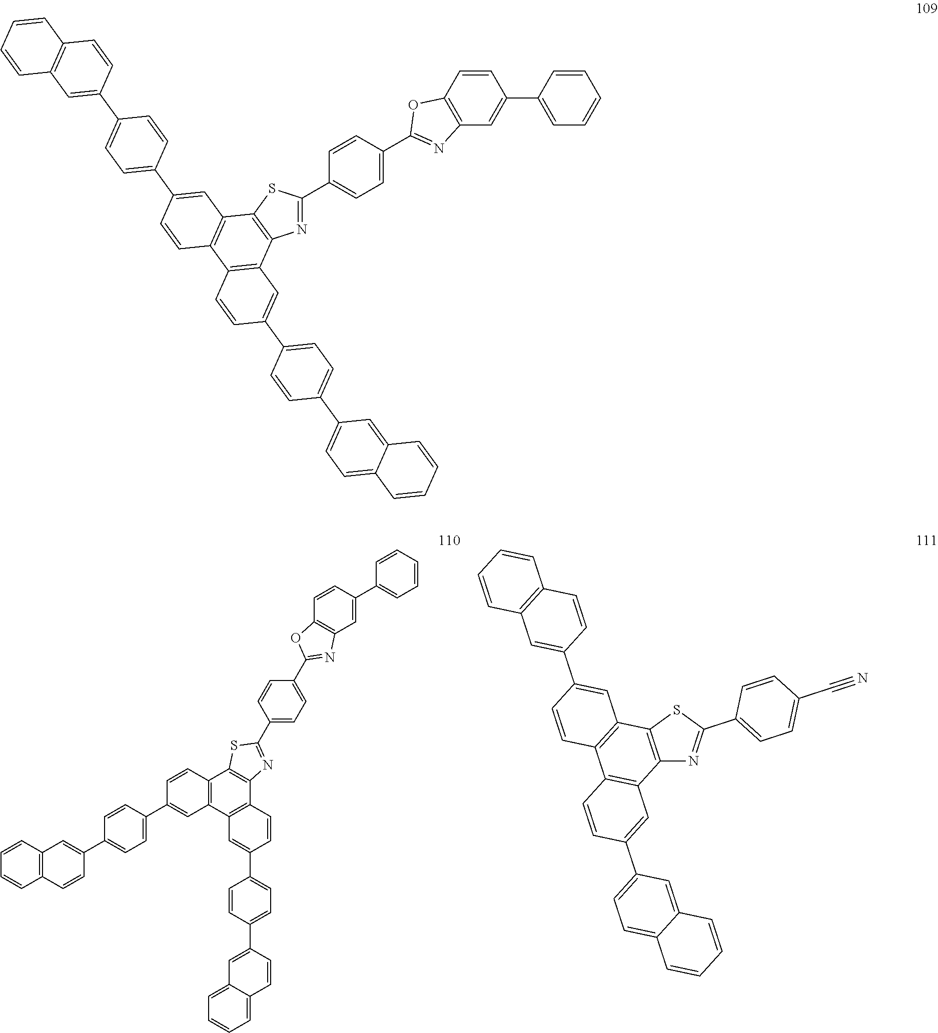 [SMILES: c1ccc(-c2ccc3oc(-c4ccc(-c5nc6c7cc(-c8ccc(-c9ccc%10ccccc%10c9)cc8)ccc7c7ccc(-c8ccc(-c9ccc%10ccccc%10c9)cc8)cc7c6s5)cc4)nc3c2)cc1 ; c1ccc(-c2ccc3oc(-c4ccc(-c5nc6c7ccc(-c8ccc(-c9ccc%10ccccc%10c9)cc8)cc7c7cc(-c8ccc(-c9ccc%10ccccc%10c9)cc8)ccc7c6s5)cc4)nc3c2)cc1 ; N#Cc1ccc(-c2nc3c4cc(-c5ccc6ccccc6c5)ccc4c4ccc(-c5ccc6ccccc6c5)cc4c3s2)cc1]

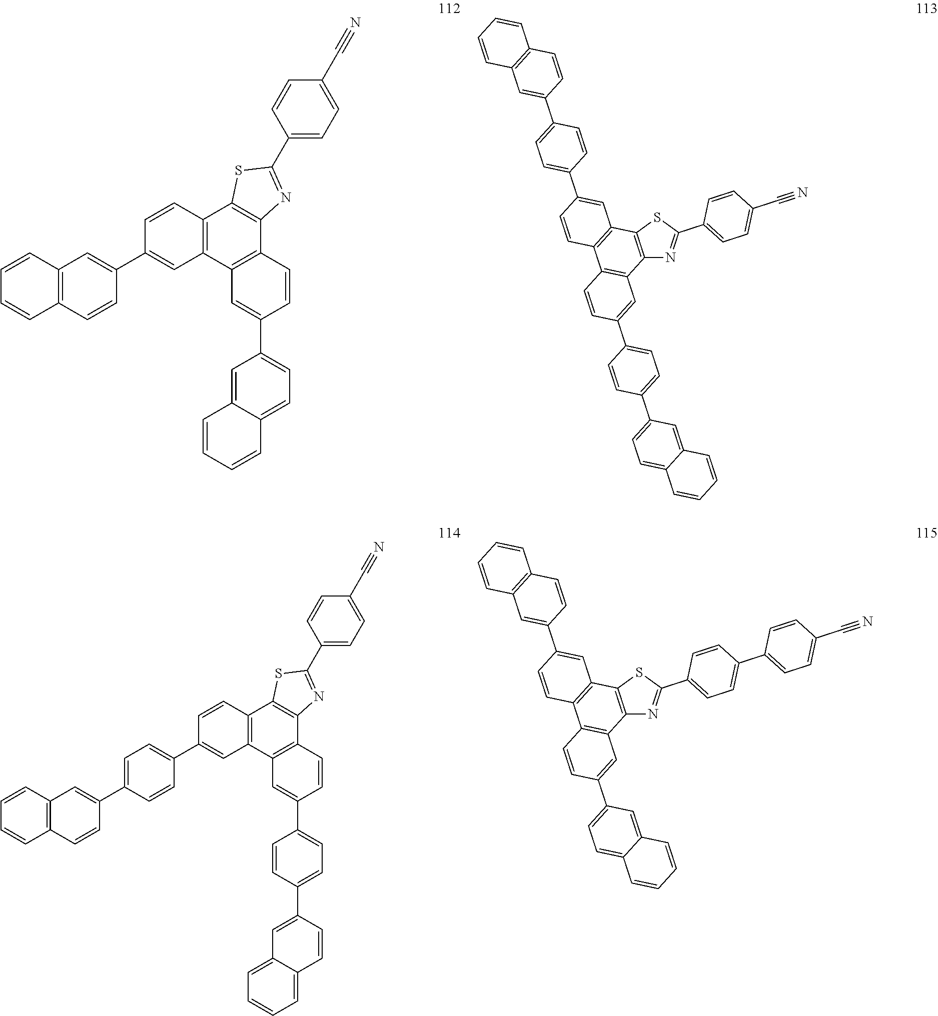 [SMILES: N#Cc1ccc(-c2nc3c4ccc(-c5ccc6ccccc6c5)cc4c4cc(-c5ccc6ccccc6c5)ccc4c3s2)cc1 ; N#Cc1ccc(-c2nc3c4cc(-c5ccc(-c6ccc7ccccc7c6)cc5)ccc4c4ccc(-c5ccc(-c6ccc7ccccc7c6)cc5)cc4c3s2)cc1 ; N#Cc1ccc(-c2nc3c4ccc(-c5ccc(-c6ccc7ccccc7c6)cc5)cc4c4cc(-c5ccc(-c6ccc7ccccc7c6)cc5)ccc4c3s2)cc1 ; N#Cc1ccc(-c2ccc(-c3nc4c5cc(-c6ccc7ccccc7c6)ccc5c5ccc(-c6ccc7ccccc7c6)cc5c4s3)cc2)cc1]

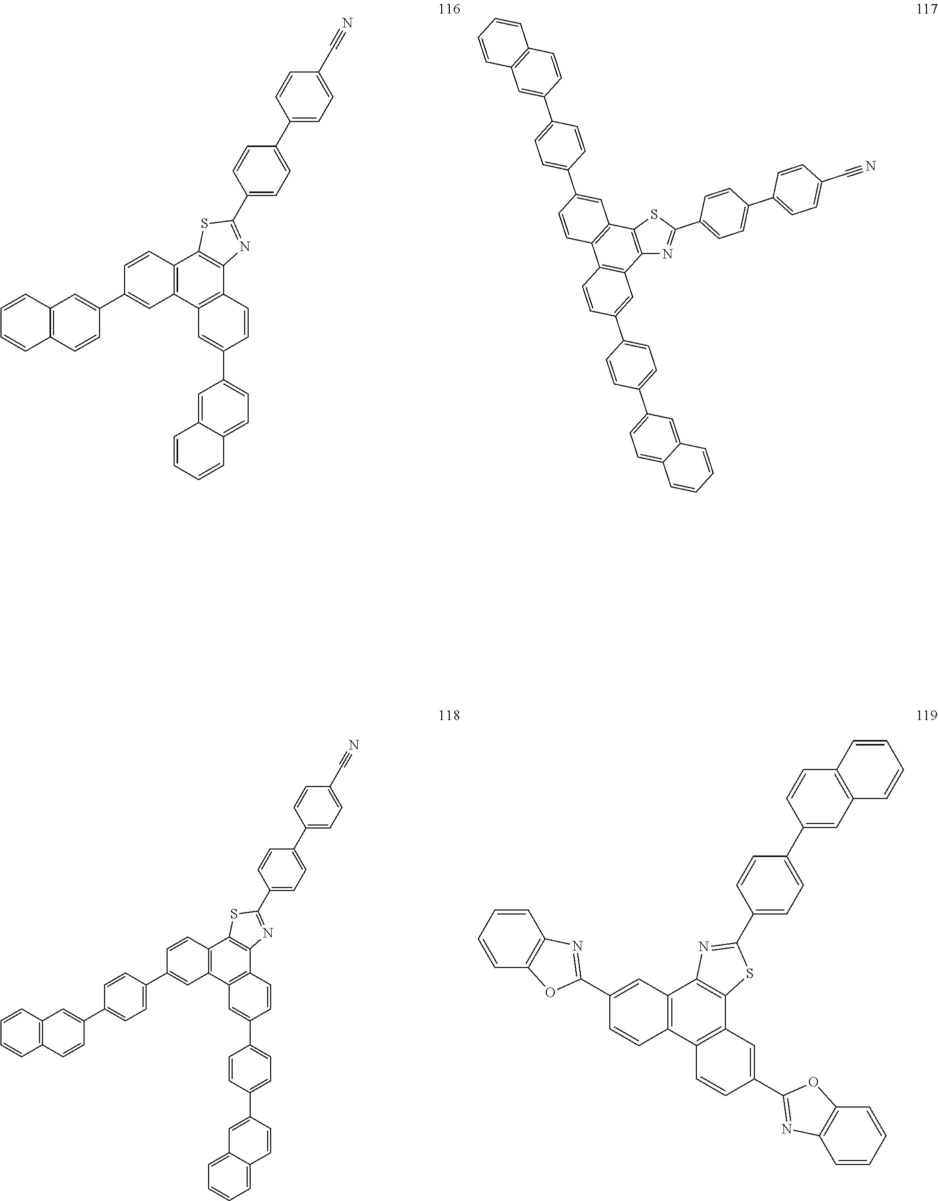 [SMILES: N#Cc1ccc(-c2ccc(-c3nc4c5ccc(-c6ccc7ccccc7c6)cc5c5cc(-c6ccc7ccccc7c6)ccc5c4s3)cc2)cc1 ; N#Cc1ccc(-c2ccc(-c3nc4c5cc(-c6ccc(-c7ccc8ccccc8c7)cc6)ccc5c5ccc(-c6ccc(-c7ccc8ccccc8c7)cc6)cc5c4s3)cc2)cc1 ; N#Cc1ccc(-c2ccc(-c3nc4c5ccc(-c6ccc(-c7ccc8ccccc8c7)cc6)cc5c5cc(-c6ccc(-c7ccc8ccccc8c7)cc6)ccc5c4s3)cc2)cc1 ; c1ccc2cc(-c3ccc(-c4nc5c6cc(-c7nc8ccccc8o7)ccc6c6ccc(-c7nc8ccccc8o7)cc6c5s4)cc3)ccc2c1]

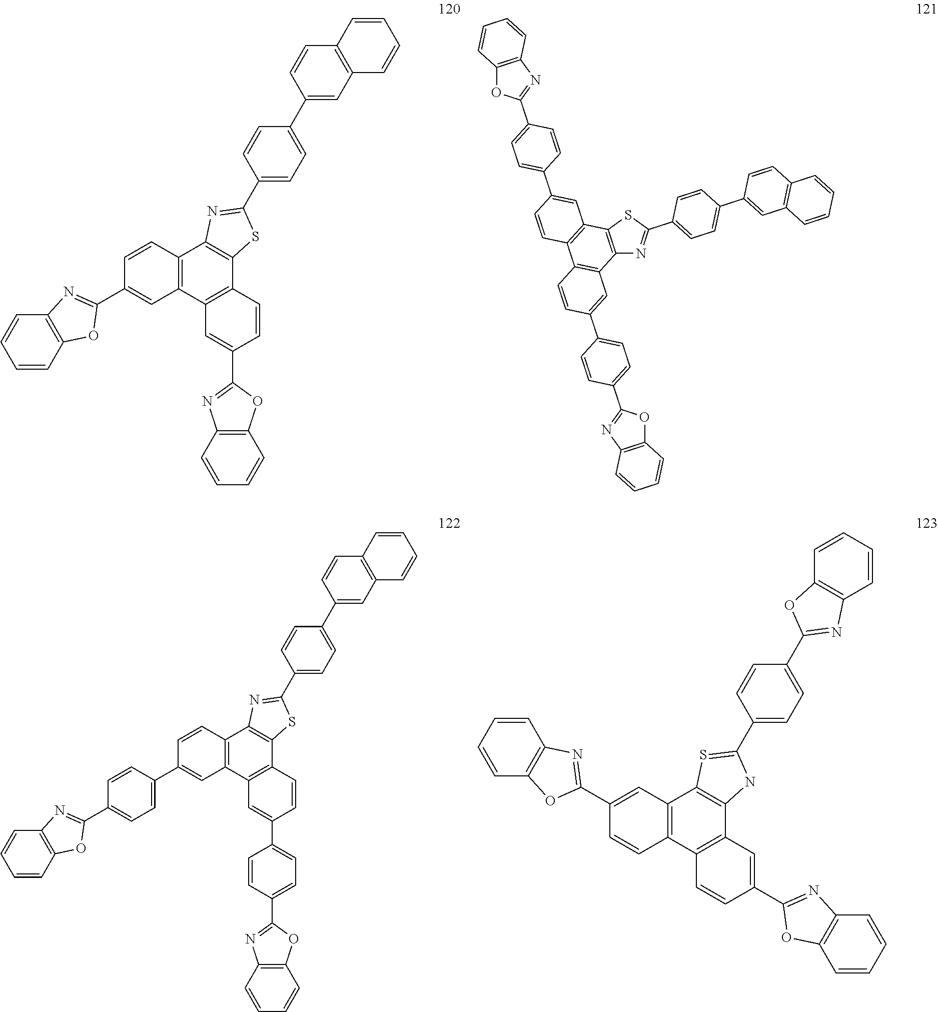

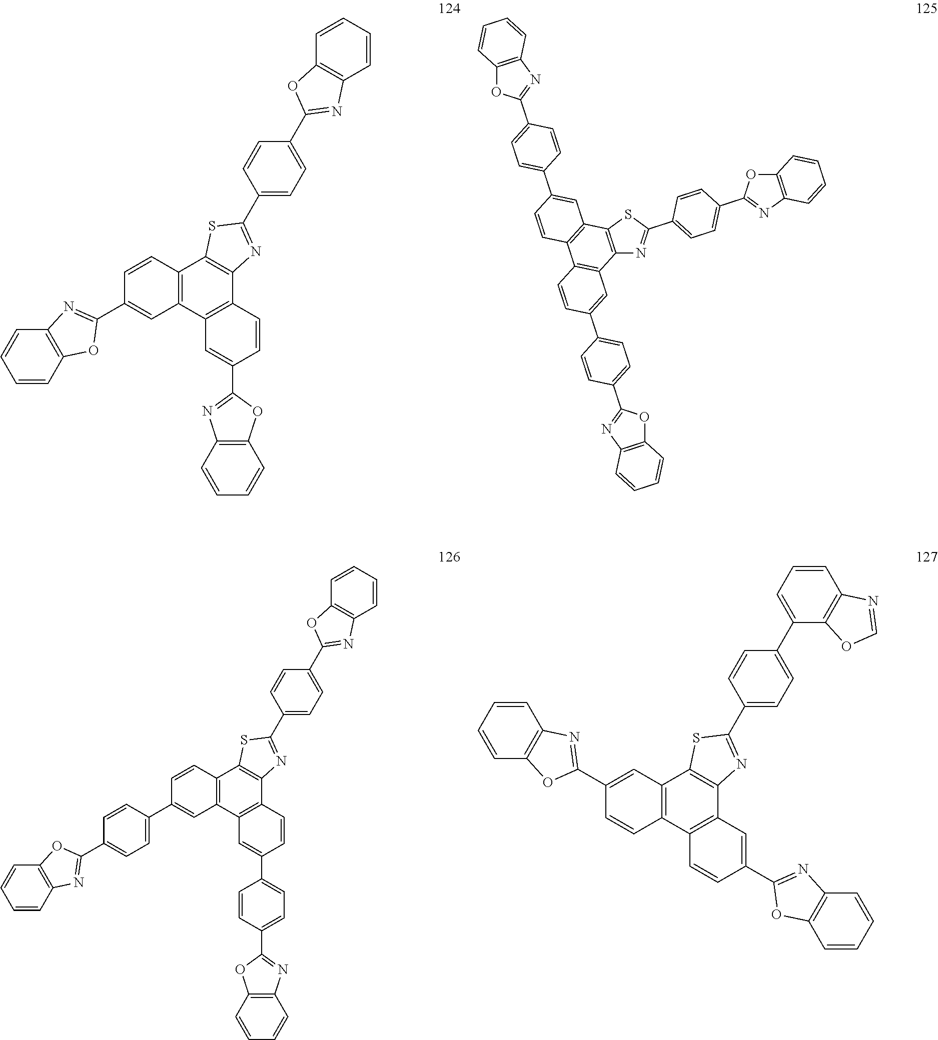 [SMILES: c1ccc2oc(-c3ccc(-c4nc5c6ccc(-c7nc8ccccc8o7)cc6c6cc(-c7nc8ccccc8o7)ccc6c5s4)cc3)nc2c1 ; c1ccc2oc(-c3ccc(-c4ccc5c6ccc(-c7ccc(-c8nc9ccccc9o8)cc7)cc6c6sc(-c7ccc(-c8nc9ccccc9o8)cc7)nc6c5c4)cc3)nc2c1 ; c1ccc2oc(-c3ccc(-c4ccc5c(c4)c4cc(-c6ccc(-c7nc8ccccc8o7)cc6)ccc4c4sc(-c6ccc(-c7nc8ccccc8o7)cc6)nc54)cc3)nc2c1 ; c1ccc2oc(-c3ccc4c5ccc(-c6nc7ccccc7o6)cc5c5sc(-c6ccc(-c7cccc8ncoc78)cc6)nc5c4c3)nc2c1]

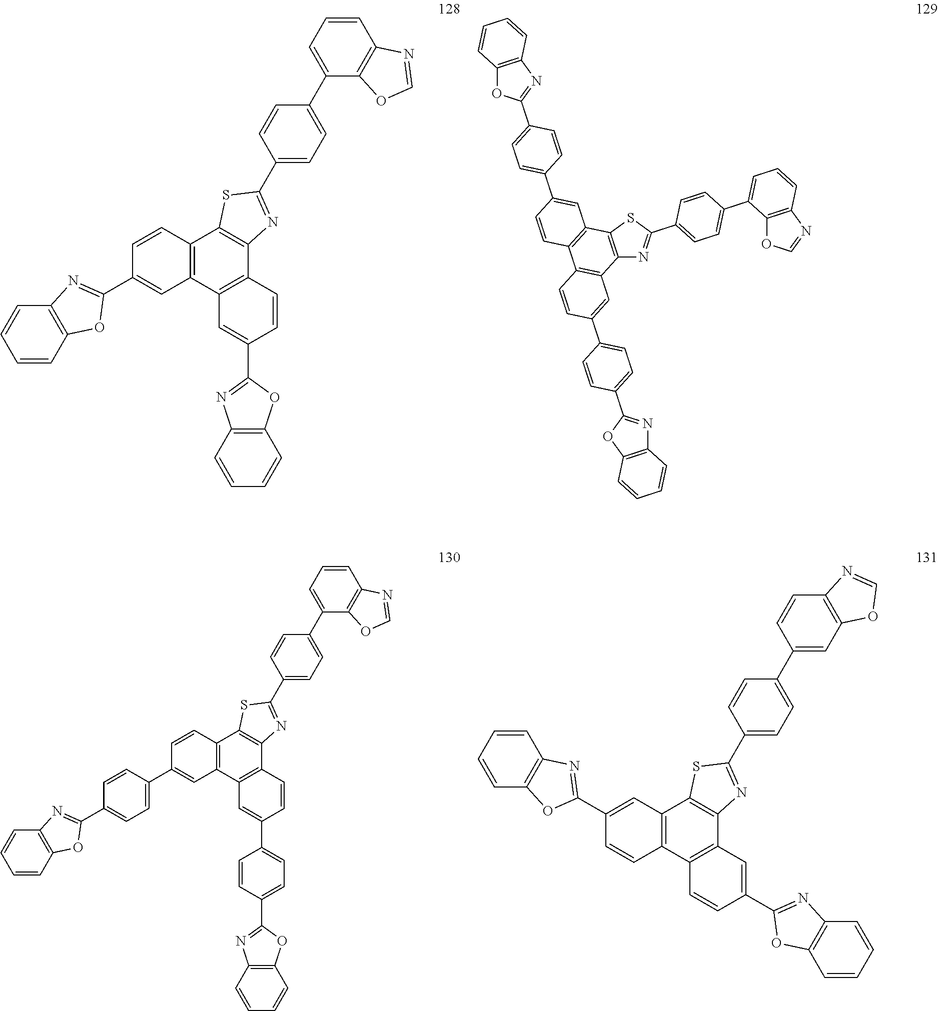 [SMILES: c1ccc2oc(-c3ccc4c(c3)c3cc(-c5nc6ccccc6o5)ccc3c3sc(-c5ccc(-c6cccc7ncoc67)cc5)nc43)nc2c1 ; c1ccc2oc(-c3ccc(-c4ccc5c6ccc(-c7ccc(-c8nc9ccccc9o8)cc7)cc6c6sc(-c7ccc(-c8cccc9ncoc89)cc7)nc6c5c4)cc3)nc2c1 ; c1ccc2oc(-c3ccc(-c4ccc5c(c4)c4cc(-c6ccc(-c7nc8ccccc8o7)cc6)ccc4c4sc(-c6ccc(-c7cccc8ncoc78)cc6)nc54)cc3)nc2c1 ; c1ccc2oc(-c3ccc4c5ccc(-c6nc7ccccc7o6)cc5c5sc(-c6ccc(-c7ccc8ncoc8c7)cc6)nc5c4c3)nc2c1]

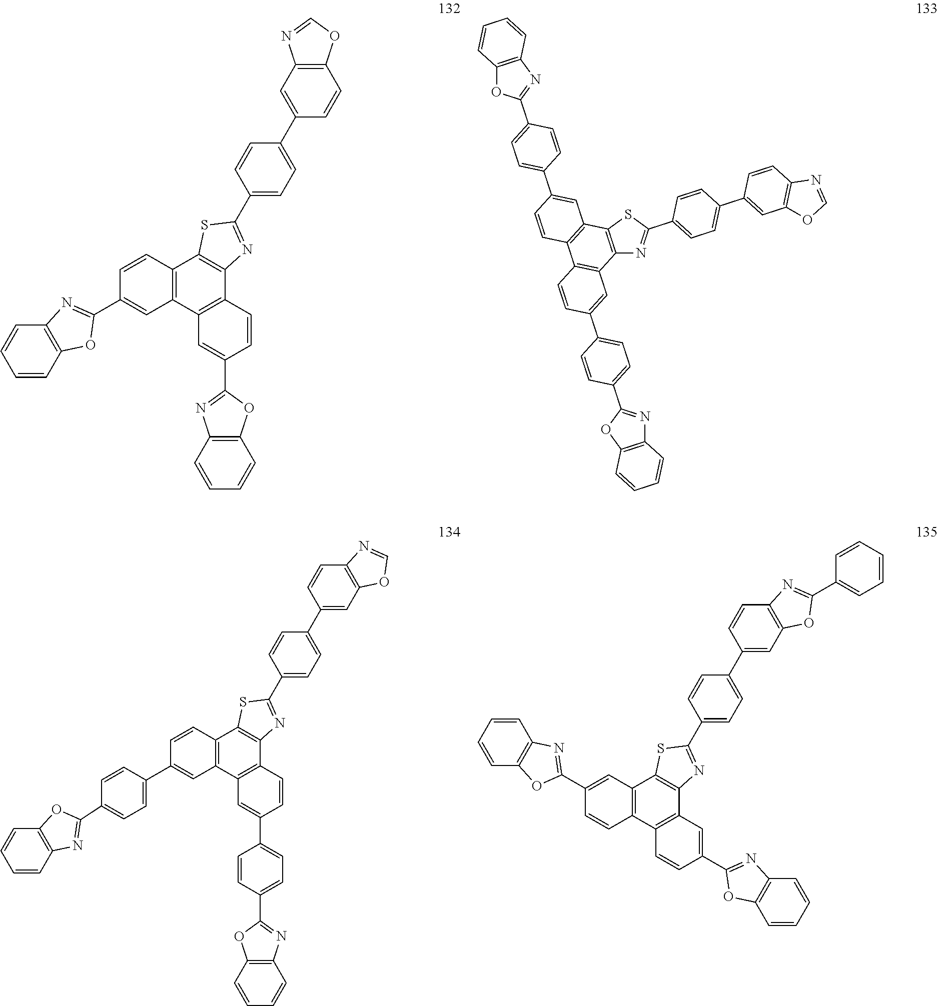 [SMILES: c1ccc2oc(-c3ccc4c(c3)c3cc(-c5nc6ccccc6o5)ccc3c3sc(-c5ccc(-c6ccc7ocnc7c6)cc5)nc43)nc2c1 ; c1ccc2oc(-c3ccc(-c4ccc5c6ccc(-c7ccc(-c8nc9ccccc9o8)cc7)cc6c6sc(-c7ccc(-c8ccc9ncoc9c8)cc7)nc6c5c4)cc3)nc2c1 ; c1ccc2oc(-c3ccc(-c4ccc5c(c4)c4cc(-c6ccc(-c7nc8ccccc8o7)cc6)ccc4c4sc(-c6ccc(-c7ccc8ncoc8c7)cc6)nc54)cc3)nc2c1 ; c1ccc(-c2nc3ccc(-c4ccc(-c5nc6c7cc(-c8nc9ccccc9o8)ccc7c7ccc(-c8nc9ccccc9o8)cc7c6s5)cc4)cc3o2)cc1]

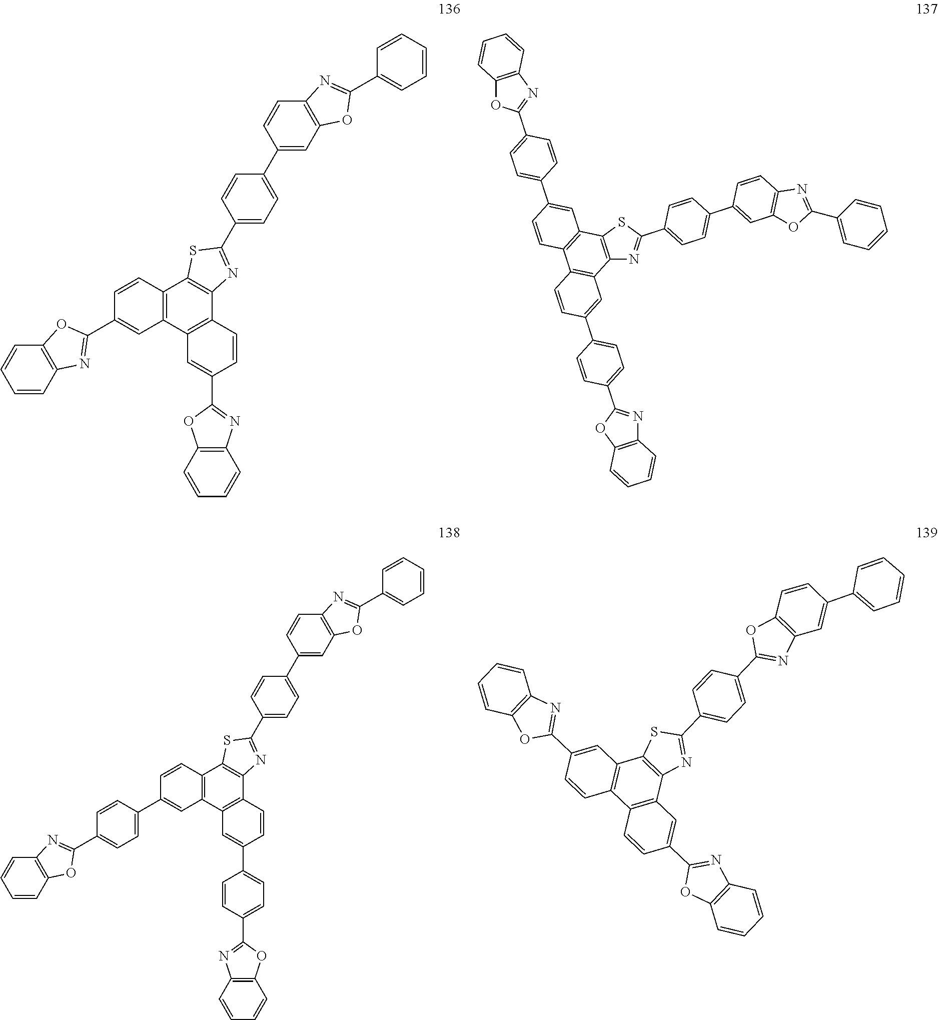 [SMILES: c1ccc(-c2nc3ccc(-c4ccc(-c5nc6c7ccc(-c8nc9ccccc9o8)cc7c7cc(-c8nc9ccccc9o8)ccc7c6s5)cc4)cc3o2)cc1 ; c1ccc(-c2nc3ccc(-c4ccc(-c5nc6c7cc(-c8ccc(-c9nc%10ccccc%10o9)cc8)ccc7c7ccc(-c8ccc(-c9nc%10ccccc%10o9)cc8)cc7c6s5)cc4)cc3o2)cc1 ; c1ccc(-c2nc3ccc(-c4ccc(-c5nc6c7ccc(-c8ccc(-c9nc%10ccccc%10o9)cc8)cc7c7cc(-c8ccc(-c9nc%10ccccc%10o9)cc8)ccc7c6s5)cc4)cc3o2)cc1 ; c1ccc(-c2ccc3oc(-c4ccc(-c5nc6c7cc(-c8nc9ccccc9o8)ccc7c7ccc(-c8nc9ccccc9o8)cc7c6s5)cc4)nc3c2)cc1]

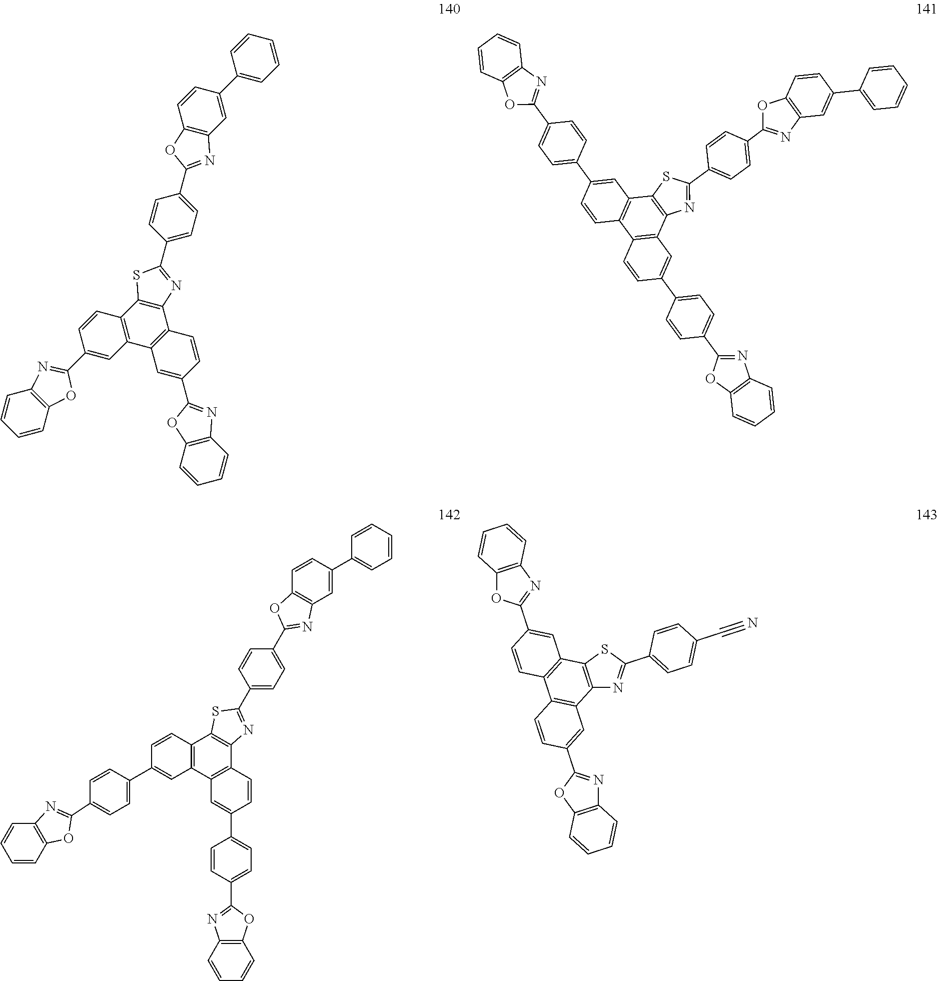 [SMILES: c1ccc(-c2ccc3oc(-c4ccc(-c5nc6c7ccc(-c8nc9ccccc9o8)cc7c7cc(-c8nc9ccccc9o8)ccc7c6s5)cc4)nc3c2)cc1 ; c1ccc(-c2ccc3oc(-c4ccc(-c5nc6c7cc(-c8ccc(-c9nc%10ccccc%10o9)cc8)ccc7c7ccc(-c8ccc(-c9nc%10ccccc%10o9)cc8)cc7c6s5)cc4)nc3c2)cc1 ; c1ccc(-c2ccc3oc(-c4ccc(-c5nc6c7ccc(-c8ccc(-c9nc%10ccccc%10o9)cc8)cc7c7cc(-c8ccc(-c9nc%10ccccc%10o9)cc8)ccc7c6s5)cc4)nc3c2)cc1 ; N#Cc1ccc(-c2nc3c4cc(-c5nc6ccccc6o5)ccc4c4ccc(-c5nc6ccccc6o5)cc4c3s2)cc1]

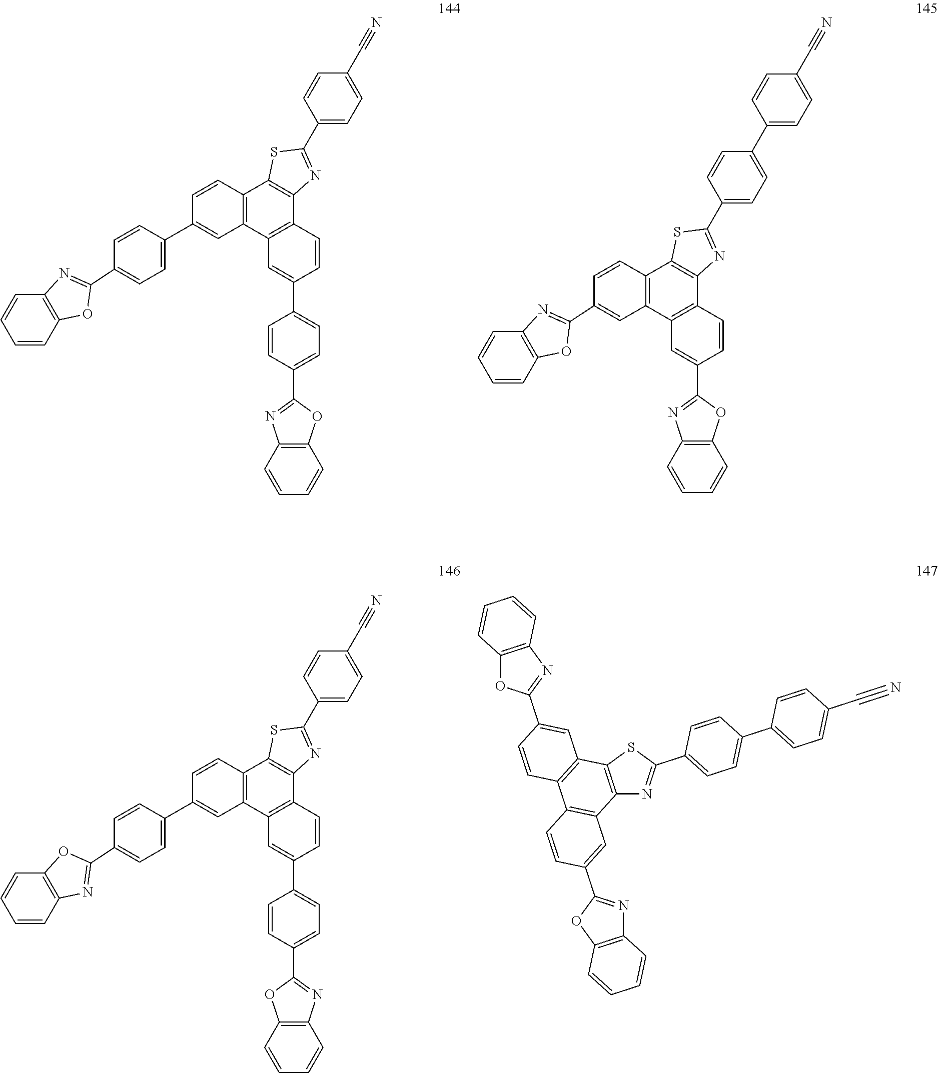 [SMILES: N#Cc1ccc(-c2nc3c4ccc(-c5ccc(-c6nc7ccccc7o6)cc5)cc4c4cc(-c5ccc(-c6nc7ccccc7o6)cc5)ccc4c3s2)cc1 ; N#Cc1ccc(-c2ccc(-c3nc4c5ccc(-c6nc7ccccc7o6)cc5c5cc(-c6nc7ccccc7o6)ccc5c4s3)cc2)cc1 ; N#Cc1ccc(-c2nc3c4ccc(-c5ccc(-c6nc7ccccc7o6)cc5)cc4c4cc(-c5ccc(-c6nc7ccccc7o6)cc5)ccc4c3s2)cc1 ; N#Cc1ccc(-c2ccc(-c3nc4c5cc(-c6nc7ccccc7o6)ccc5c5ccc(-c6nc7ccccc7o6)cc5c4s3)cc2)cc1]

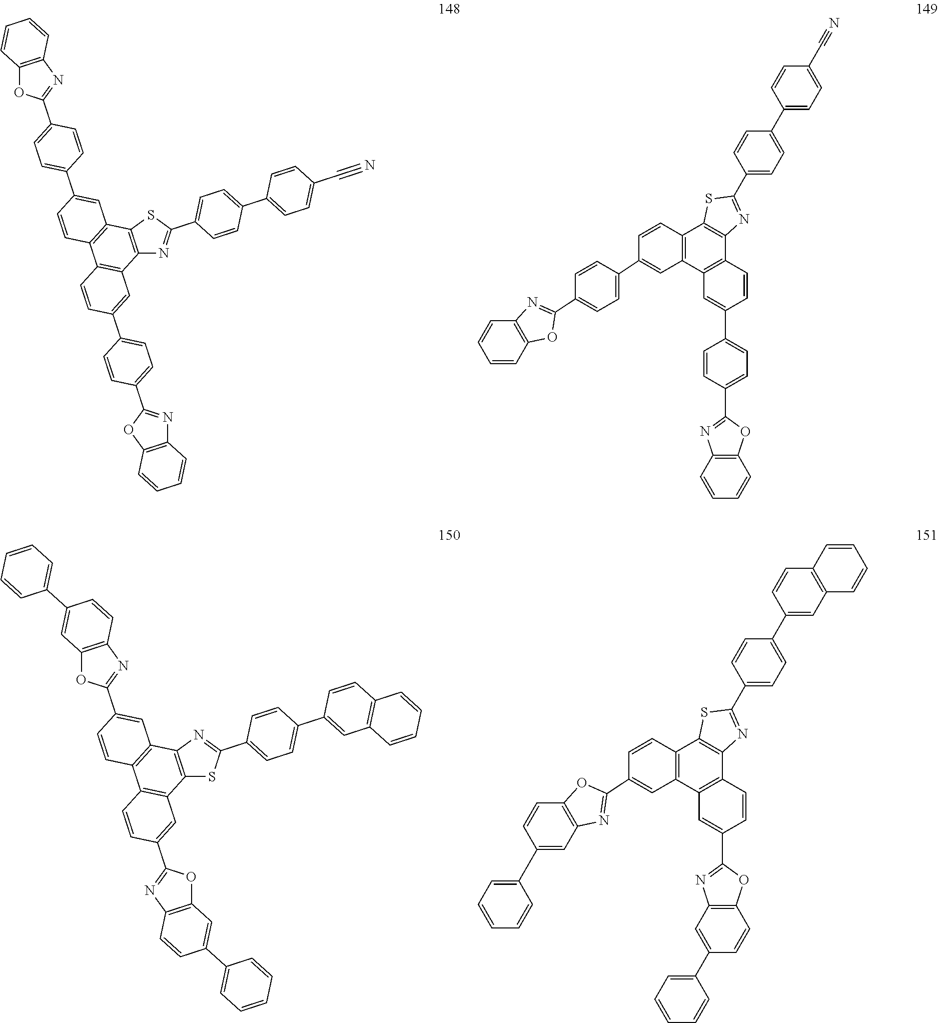 [SMILES: N#Cc1ccc(-c2ccc(-c3nc4c5cc(-c6ccc(-c7nc8ccccc8o7)cc6)ccc5c5ccc(-c6ccc(-c7nc8ccccc8o7)cc6)cc5c4s3)cc2)cc1 ; N#Cc1ccc(-c2ccc(-c3nc4c5ccc(-c6ccc(-c7nc8ccccc8o7)cc6)cc5c5cc(-c6ccc(-c7nc8ccccc8o7)cc6)ccc5c4s3)cc2)cc1 ; c1ccc(-c2ccc3nc(-c4ccc5c6ccc(-c7nc8ccc(-c9ccccc9)cc8o7)cc6c6sc(-c7ccc(-c8ccc9ccccc9c8)cc7)nc6c5c4)oc3c2)cc1 ; c1ccc(-c2ccc3oc(-c4ccc5c(c4)c4cc(-c6nc7cc(-c8ccccc8)ccc7o6)ccc4c4sc(-c6ccc(-c7ccc8ccccc8c7)cc6)nc54)nc3c2)cc1]

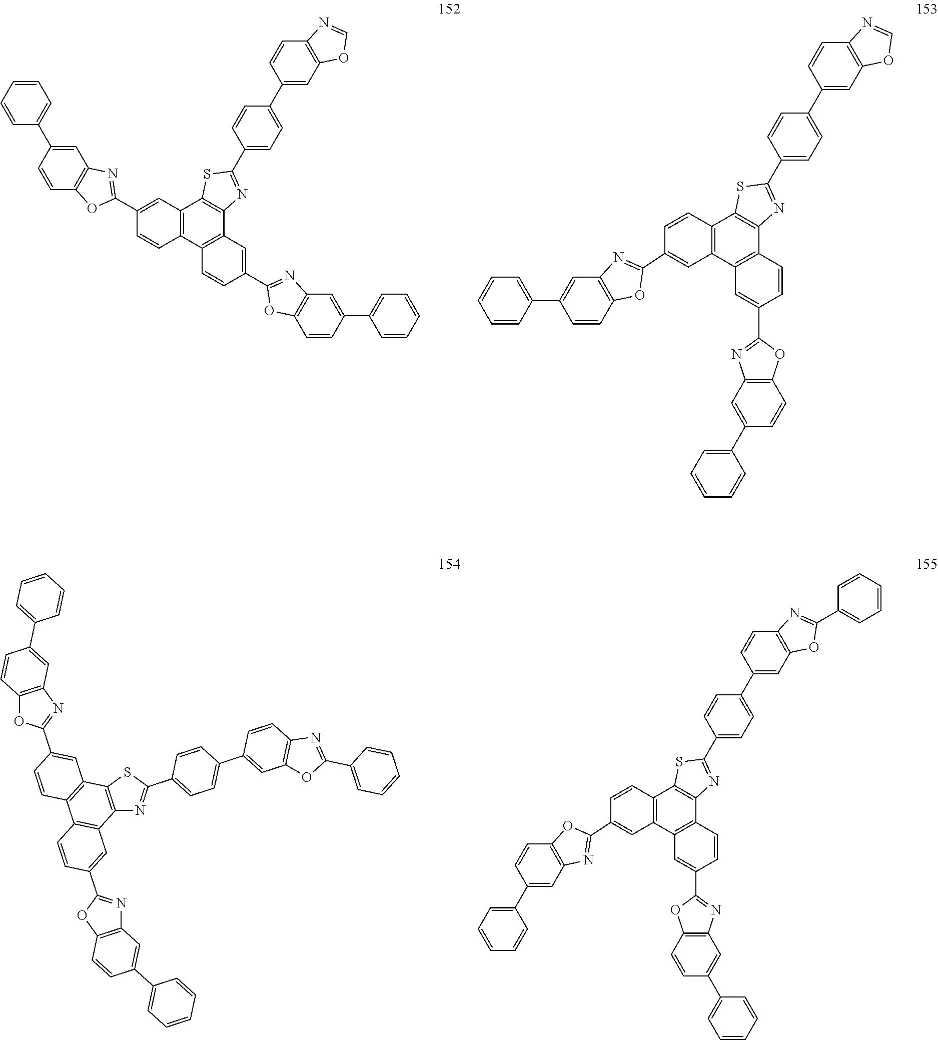 [SMILES: c1ccc(-c2ccc3oc(-c4ccc5c6ccc(-c7nc8cc(-c9ccccc9)ccc8o7)cc6c6sc(-c7ccc(-c8ccc9ncoc9c8)cc7)nc6c5c4)nc3c2)cc1 ; c1ccc(-c2ccc3oc(-c4ccc5c(c4)c4cc(-c6nc7cc(-c8ccccc8)ccc7o6)ccc4c4sc(-c6ccc(-c7ccc8ncoc8c7)cc6)nc54)nc3c2)cc1 ; c1ccc(-c2ccc3oc(-c4ccc5c6ccc(-c7nc8cc(-c9ccccc9)ccc8o7)cc6c6sc(-c7ccc(-c8ccc9nc(-c%10ccccc%10)oc9c8)cc7)nc6c5c4)nc3c2)cc1 ; c1ccc(-c2ccc3oc(-c4ccc5c(c4)c4cc(-c6nc7cc(-c8ccccc8)ccc7o6)ccc4c4sc(-c6ccc(-c7ccc8nc(-c9ccccc9)oc8c7)cc6)nc54)nc3c2)cc1]

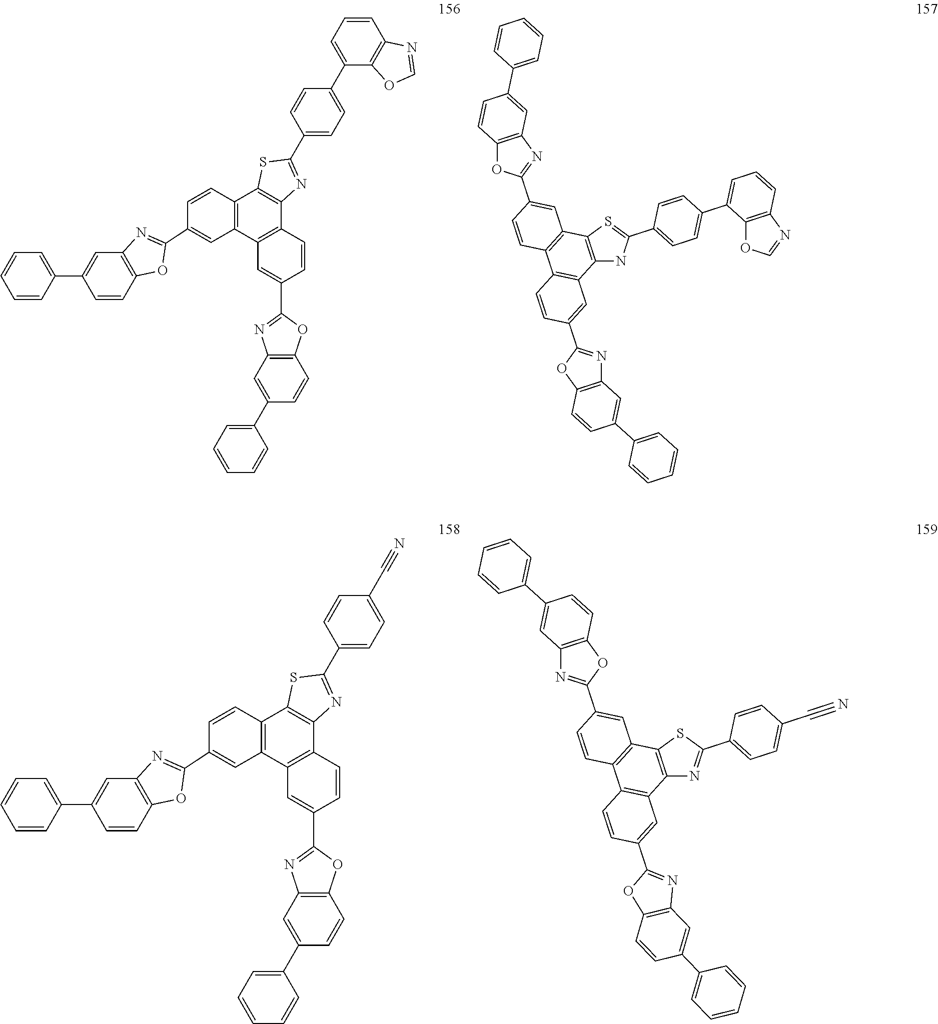

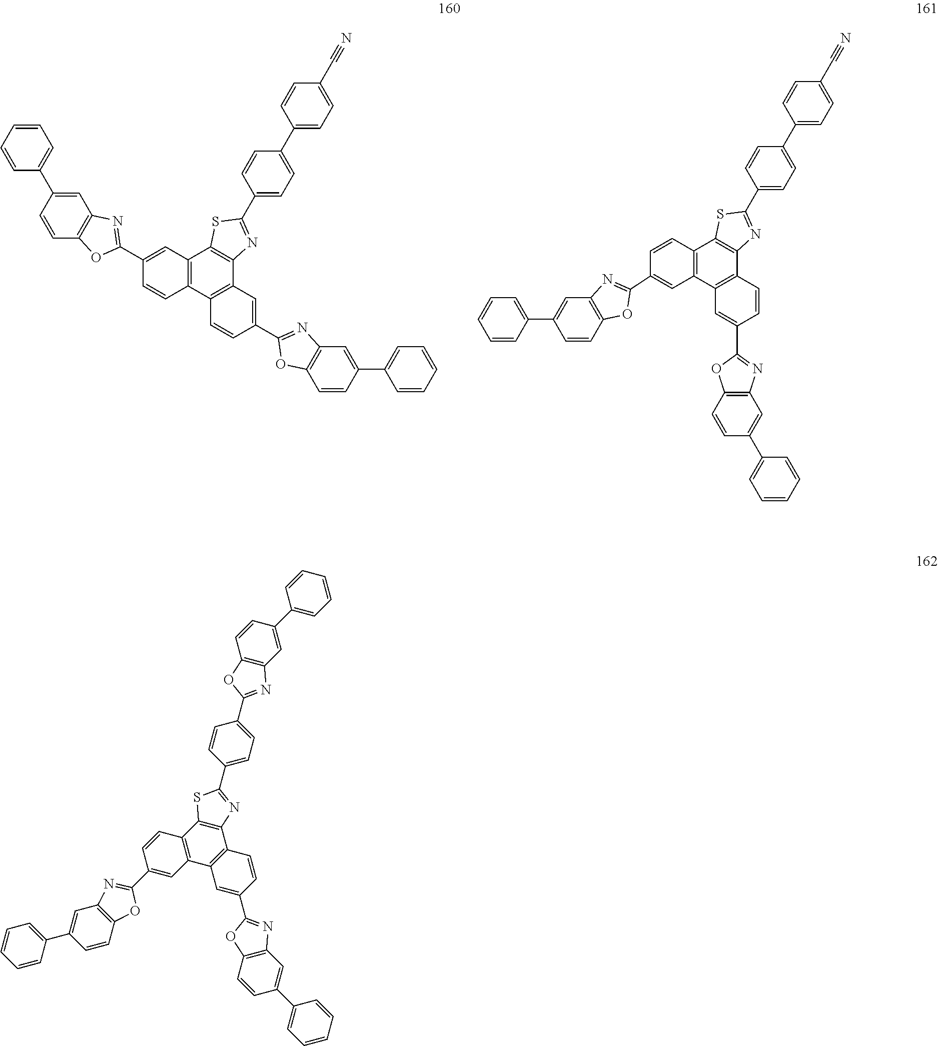 [SMILES: N#Cc1ccc(-c2ccc(-c3nc4c5cc(-c6nc7cc(-c8ccccc8)ccc7o6)ccc5c5ccc(-c6nc7cc(-c8ccccc8)ccc7o6)cc5c4s3)cc2)cc1 ; N#Cc1ccc(-c2ccc(-c3nc4c5ccc(-c6nc7cc(-c8ccccc8)ccc7o6)cc5c5cc(-c6nc7cc(-c8ccccc8)ccc7o6)ccc5c4s3)cc2)cc1 ; c1ccc(-c2ccc3oc(-c4ccc(-c5nc6c7ccc(-c8nc9cc(-c%10ccccc%10)ccc9o8)cc7c7cc(-c8nc9cc(-c%10ccccc%10)ccc9o8)ccc7c6s5)cc4)nc3c2)cc1]

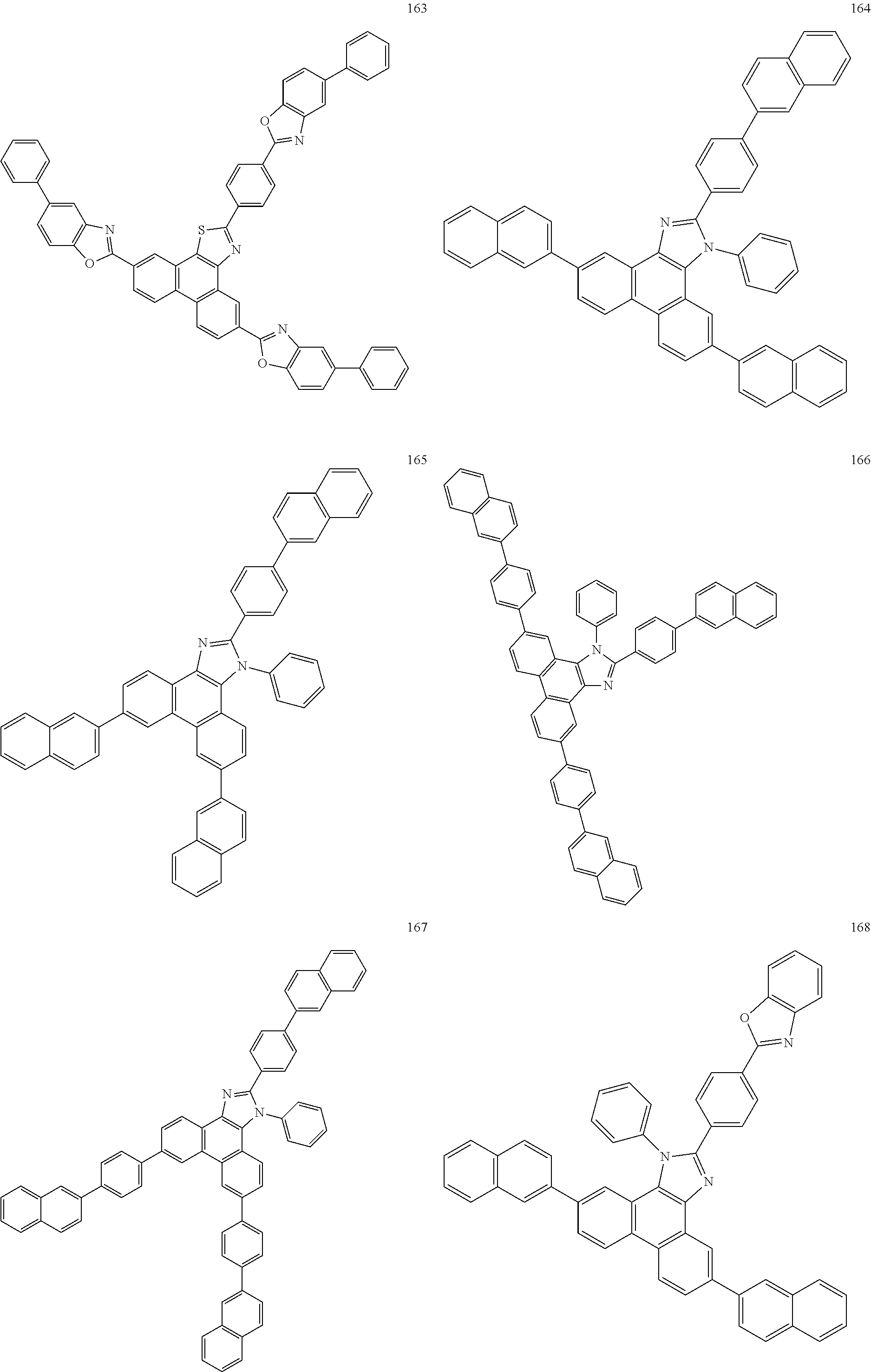

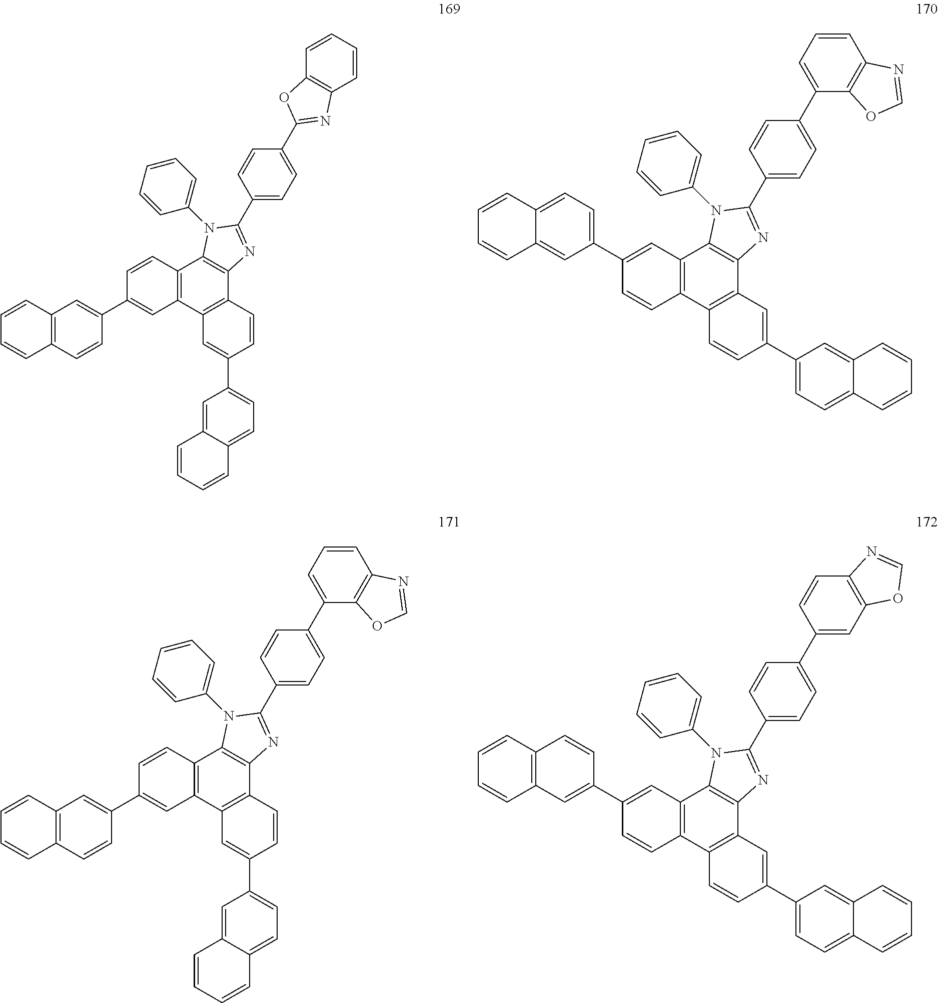 [SMILES: c1ccc(-n2c(-c3ccc(-c4nc5ccccc5o4)cc3)nc3c4ccc(-c5ccc6ccccc6c5)cc4c4cc(-c5ccc6ccccc6c5)ccc4c32)cc1 ; c1ccc(-n2c(-c3ccc(-c4cccc5ncoc45)cc3)nc3c4cc(-c5ccc6ccccc6c5)ccc4c4ccc(-c5ccc6ccccc6c5)cc4c32)cc1 ; c1ccc(-n2c(-c3ccc(-c4cccc5ncoc45)cc3)nc3c4ccc(-c5ccc6ccccc6c5)cc4c4cc(-c5ccc6ccccc6c5)ccc4c32)cc1 ; c1ccc(-n2c(-c3ccc(-c4ccc5ncoc5c4)cc3)nc3c4cc(-c5ccc6ccccc6c5)ccc4c4ccc(-c5ccc6ccccc6c5)cc4c32)cc1]

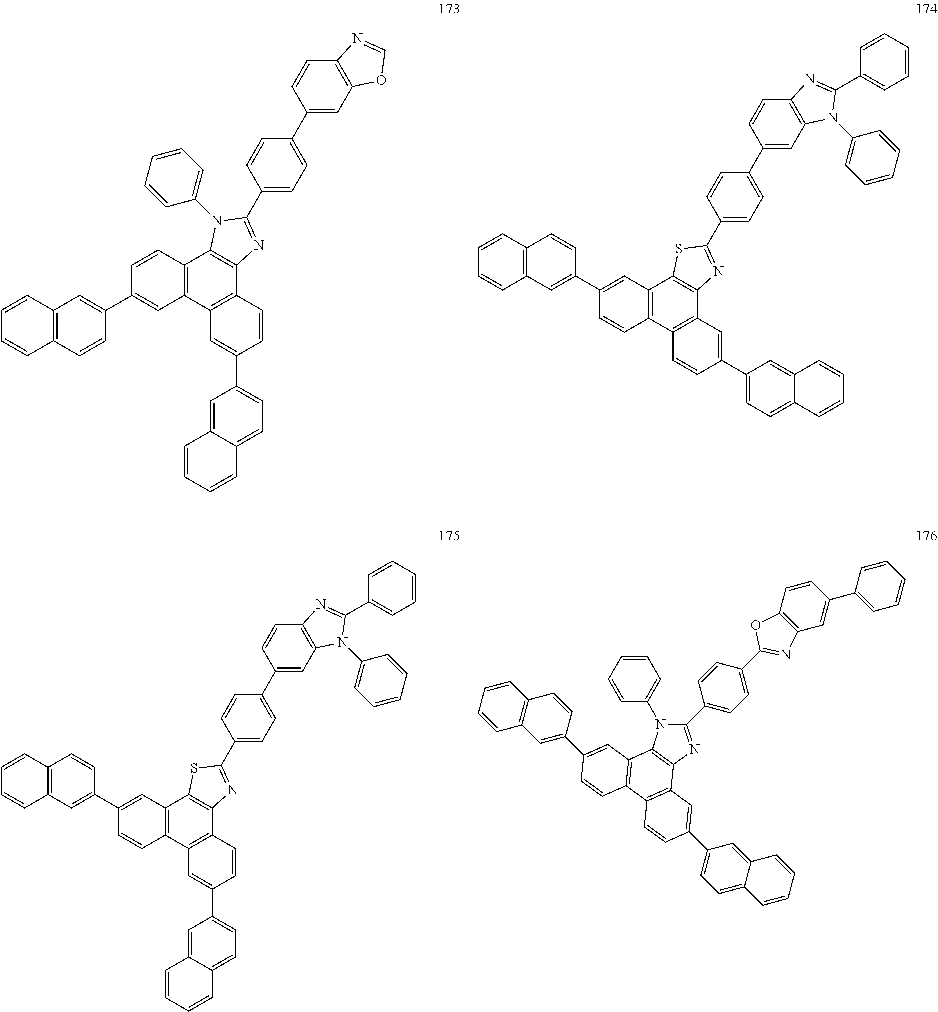 [SMILES: c1ccc(-n2c(-c3ccc(-c4ccc5ncoc5c4)cc3)nc3c4ccc(-c5ccc6ccccc6c5)cc4c4cc(-c5ccc6ccccc6c5)ccc4c32)cc1 ; c1ccc(-c2nc3ccc(-c4ccc(-c5nc6c7cc(-c8ccc9ccccc9c8)ccc7c7ccc(-c8ccc9ccccc9c8)cc7c6s5)cc4)cc3n2-c2ccccc2)cc1 ; c1ccc(-c2nc3ccc(-c4ccc(-c5nc6c7ccc(-c8ccc9ccccc9c8)cc7c7ccc(-c8ccc9ccccc9c8)cc7c6s5)cc4)cc3n2-c2ccccc2)cc1 ; c1ccc(-c2ccc3oc(-c4ccc(-c5nc6c7cc(-c8ccc9ccccc9c8)ccc7c7ccc(-c8ccc9ccccc9c8)cc7c6n5-c5ccccc5)cc4)nc3c2)cc1]

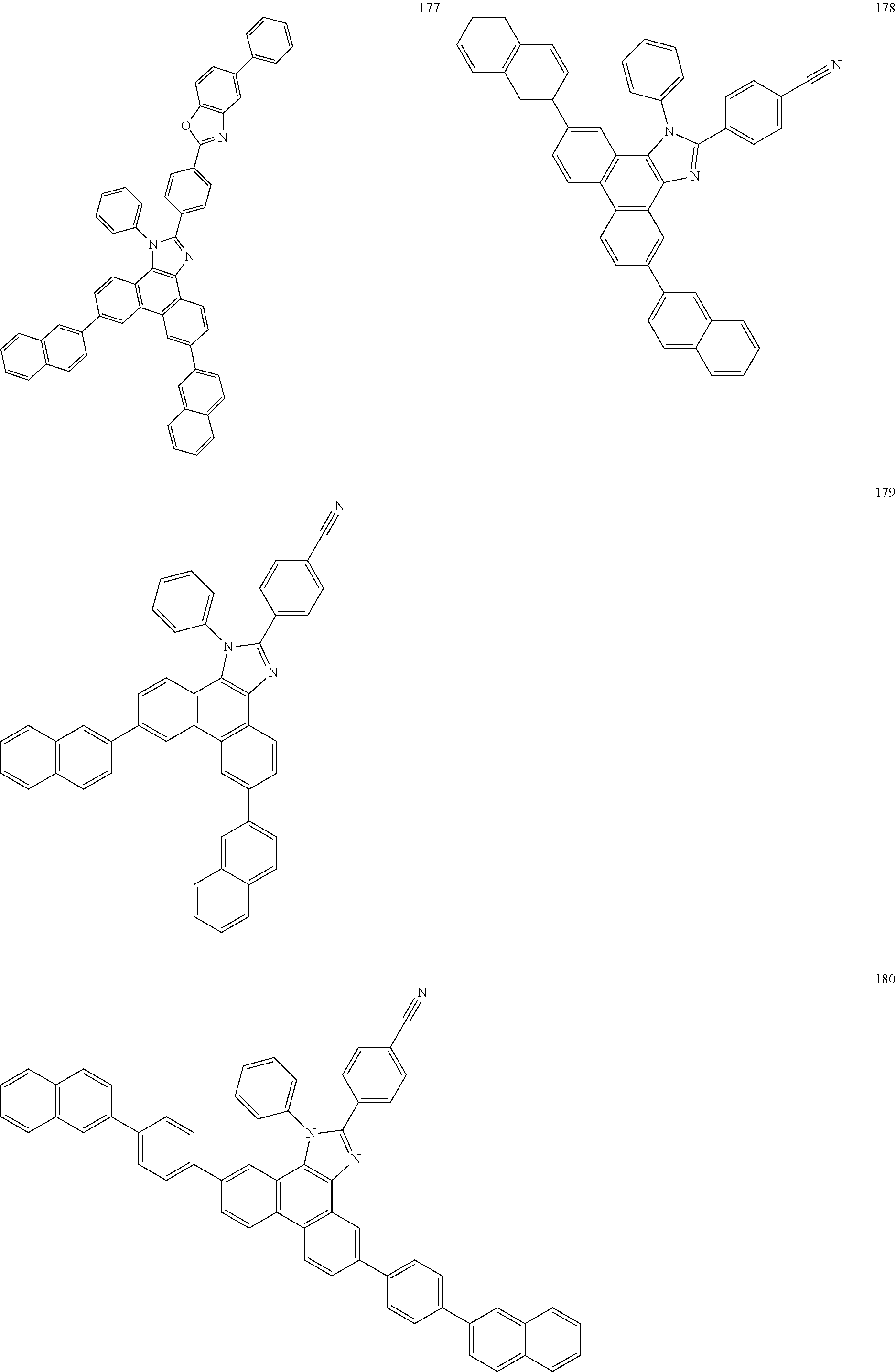 [SMILES: c1ccc(-c2ccc3oc(-c4ccc(-c5nc6c7ccc(-c8ccc9ccccc9c8)cc7c7cc(-c8ccc9ccccc9c8)ccc7c6n5-c5ccccc5)cc4)nc3c2)cc1 ; N#Cc1ccc(-c2nc3c4cc(-c5ccc6ccccc6c5)ccc4c4ccc(-c5ccc6ccccc6c5)cc4c3n2-c2ccccc2)cc1 ; N#Cc1ccc(-c2nc3c4ccc(-c5ccc6ccccc6c5)cc4c4cc(-c5ccc6ccccc6c5)ccc4c3n2-c2ccccc2)cc1 ; N#Cc1ccc(-c2nc3c4cc(-c5ccc(-c6ccc7ccccc7c6)cc5)ccc4c4ccc(-c5ccc(-c6ccc7ccccc7c6)cc5)cc4c3n2-c2ccccc2)cc1]

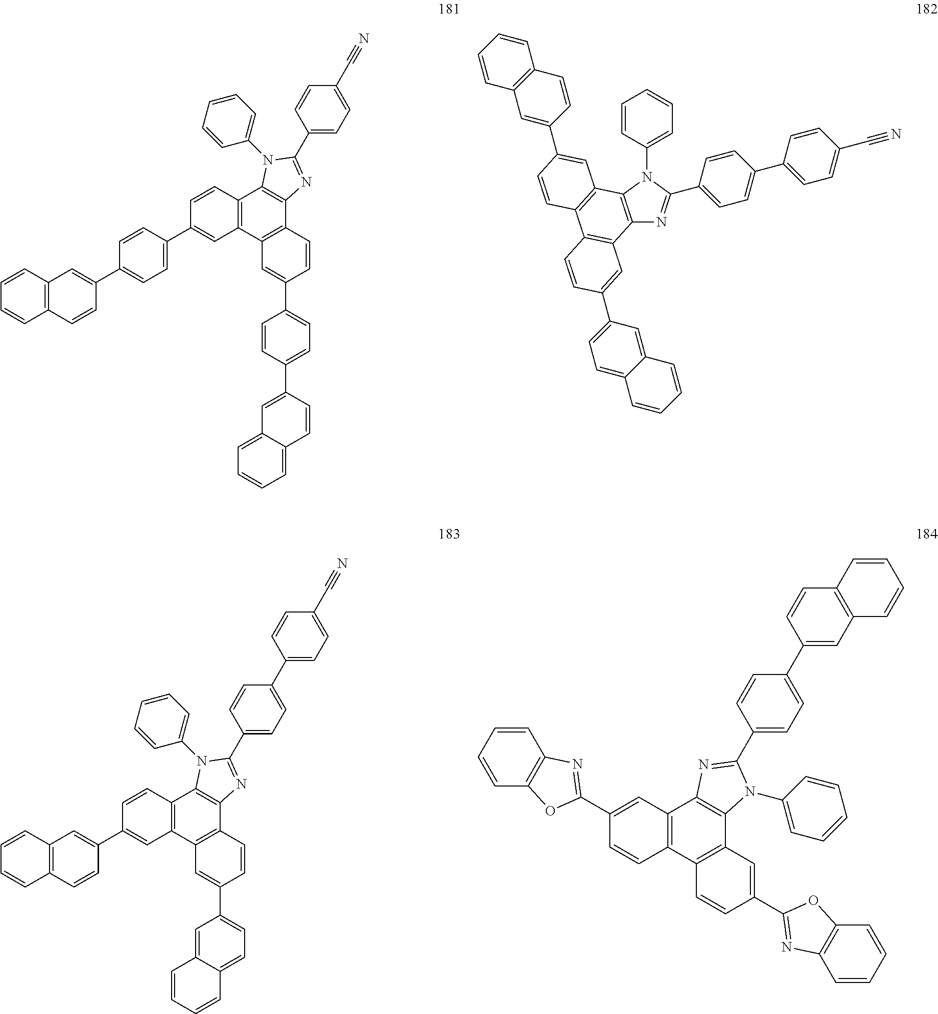 [SMILES: N#Cc1ccc(-c2nc3c4ccc(-c5ccc(-c6ccc7ccccc7c6)cc5)cc4c4cc(-c5ccc(-c6ccc7ccccc7c6)cc5)ccc4c3n2-c2ccccc2)cc1 ; N#Cc1ccc(-c2ccc(-c3nc4c5cc(-c6ccc7ccccc7c6)ccc5c5ccc(-c6ccc7ccccc7c6)cc5c4n3-c3ccccc3)cc2)cc1 ; N#Cc1ccc(-c2ccc(-c3nc4c5ccc(-c6ccc7ccccc7c6)cc5c5cc(-c6ccc7ccccc7c6)ccc5c4n3-c3ccccc3)cc2)cc1 ; c1ccc(-n2c(-c3ccc(-c4ccc5ccccc5c4)cc3)nc3c4cc(-c5nc6ccccc6o5)ccc4c4ccc(-c5nc6ccccc6o5)cc4c32)cc1]

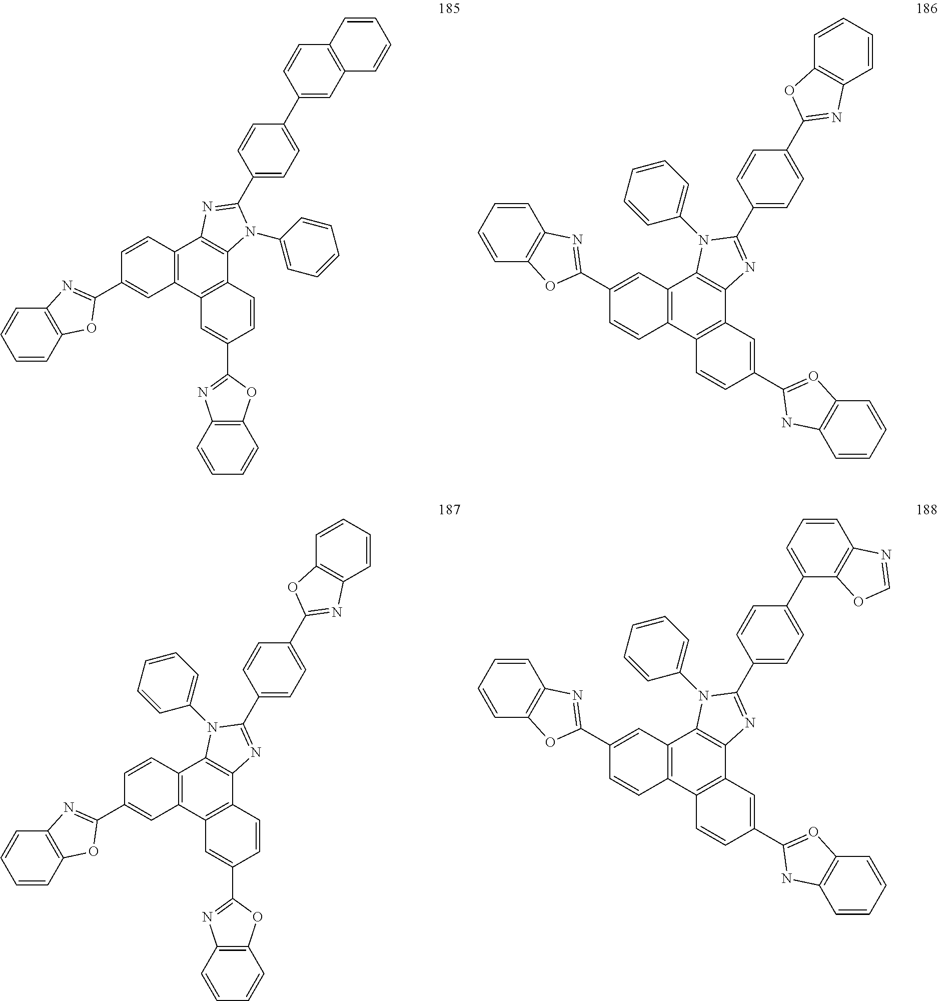

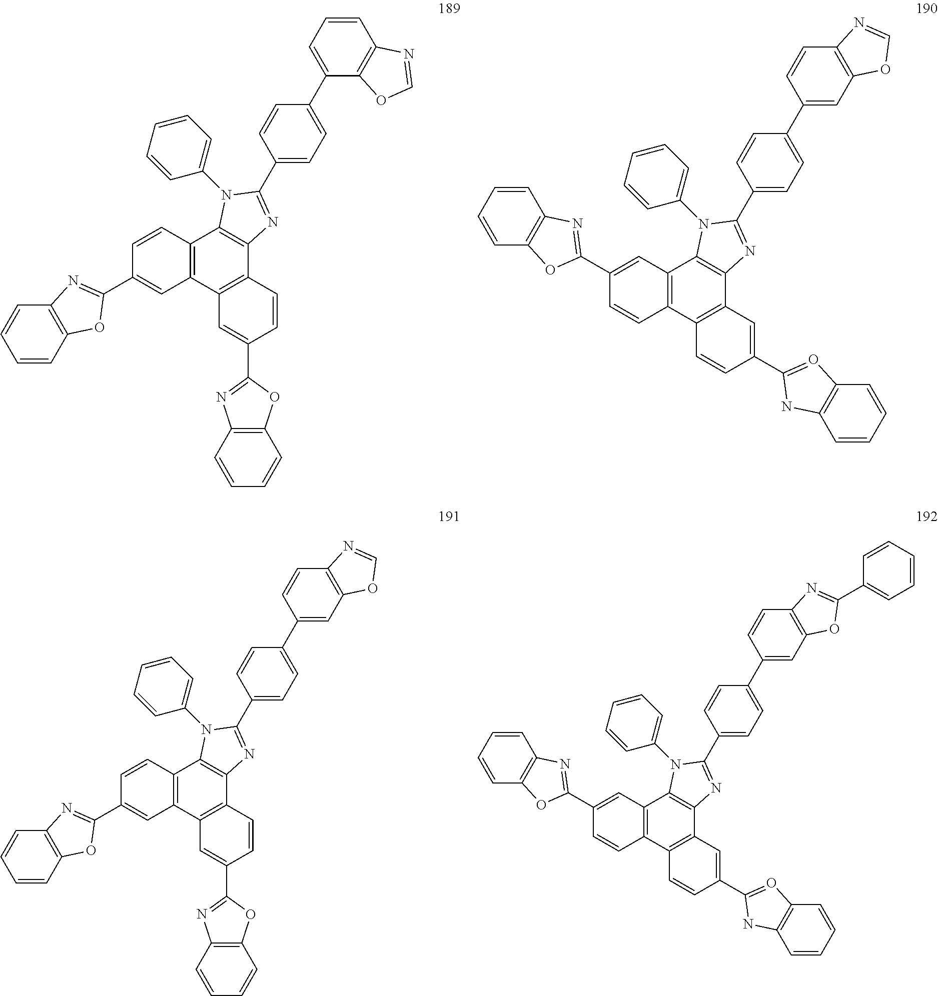

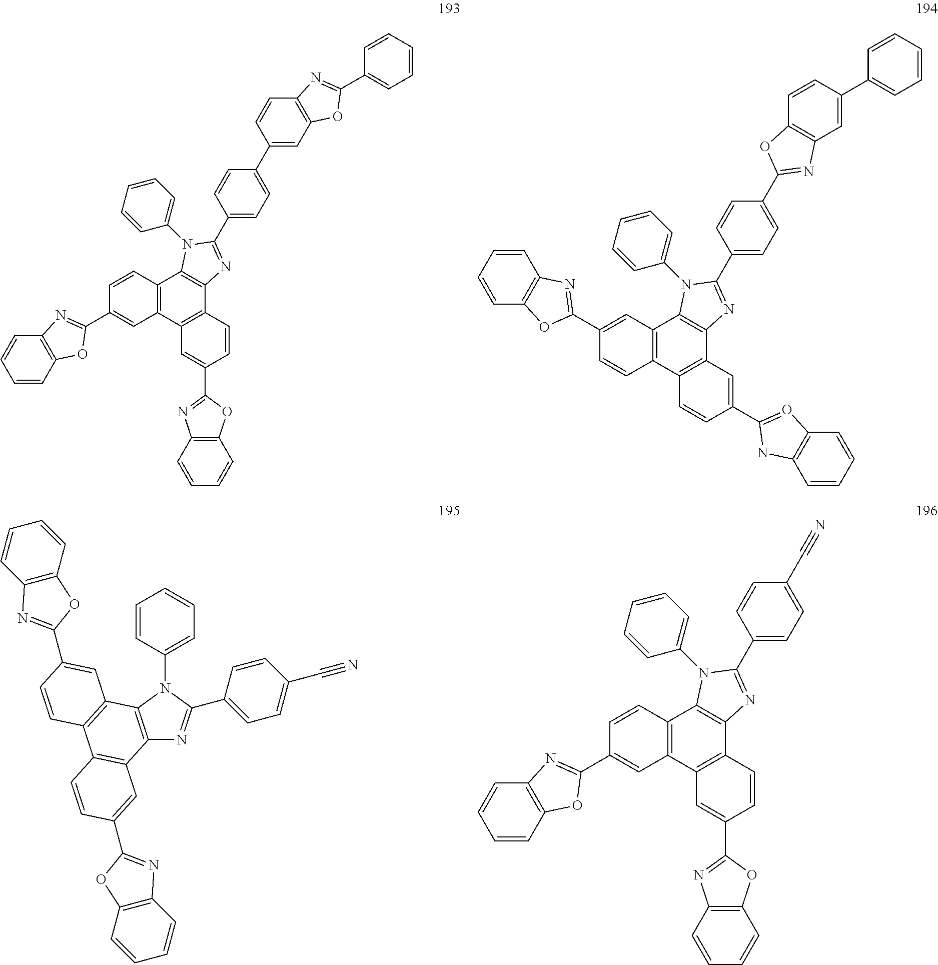

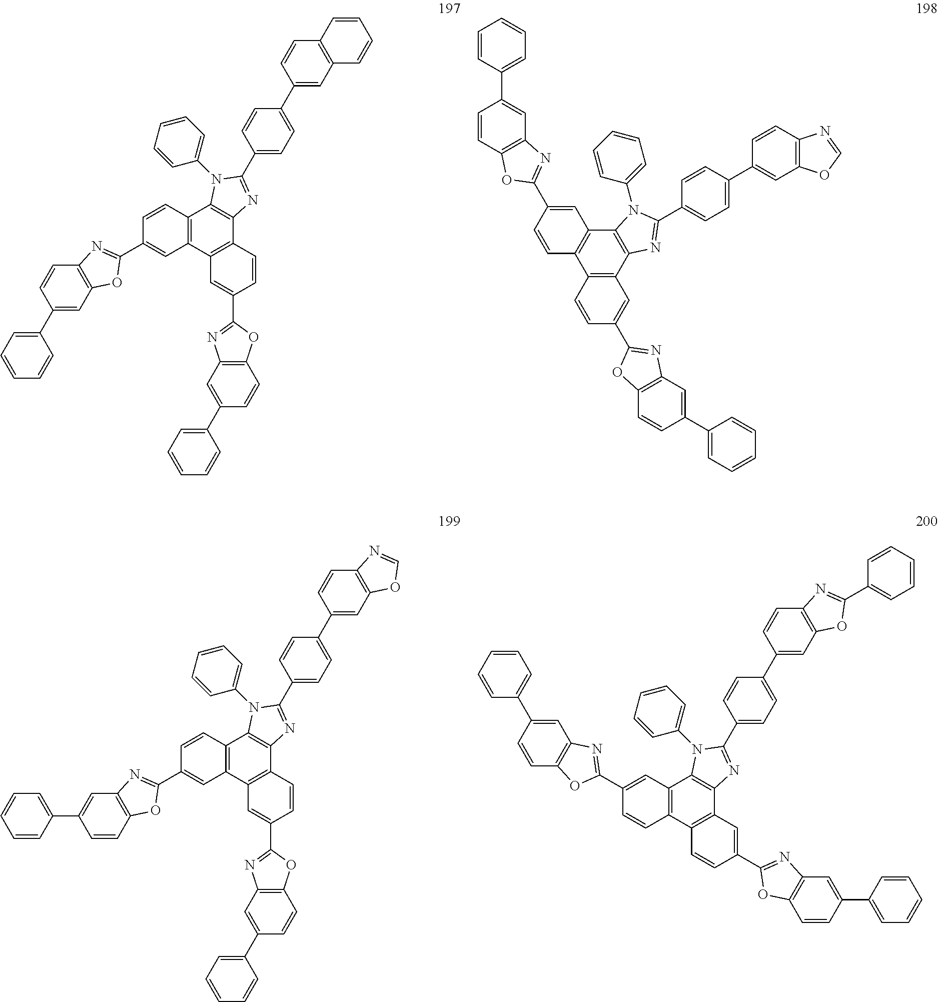 [SMILES: c1ccc(-c2ccc3oc(-c4ccc5c(c4)c4cc(-c6nc7ccc(-c8ccccc8)cc7o6)ccc4c4c5nc(-c5ccc(-c6ccc7ccccc7c6)cc5)n4-c4ccccc4)nc3c2)cc1 ; c1ccc(-c2ccc3oc(-c4ccc5c6ccc(-c7nc8cc(-c9ccccc9)ccc8o7)cc6c6c(nc(-c7ccc(-c8ccc9ncoc9c8)cc7)n6-c6ccccc6)c5c4)nc3c2)cc1 ; c1ccc(-c2ccc3oc(-c4ccc5c(c4)c4cc(-c6nc7cc(-c8ccccc8)ccc7o6)ccc4c4c5nc(-c5ccc(-c6ccc7ncoc7c6)cc5)n4-c4ccccc4)nc3c2)cc1 ; c1ccc(-c2ccc3oc(-c4ccc5c6ccc(-c7nc8cc(-c9ccccc9)ccc8o7)cc6c6c(nc(-c7ccc(-c8ccc9nc(-c%10ccccc%10)oc9c8)cc7)n6-c6ccccc6)c5c4)nc3c2)cc1]

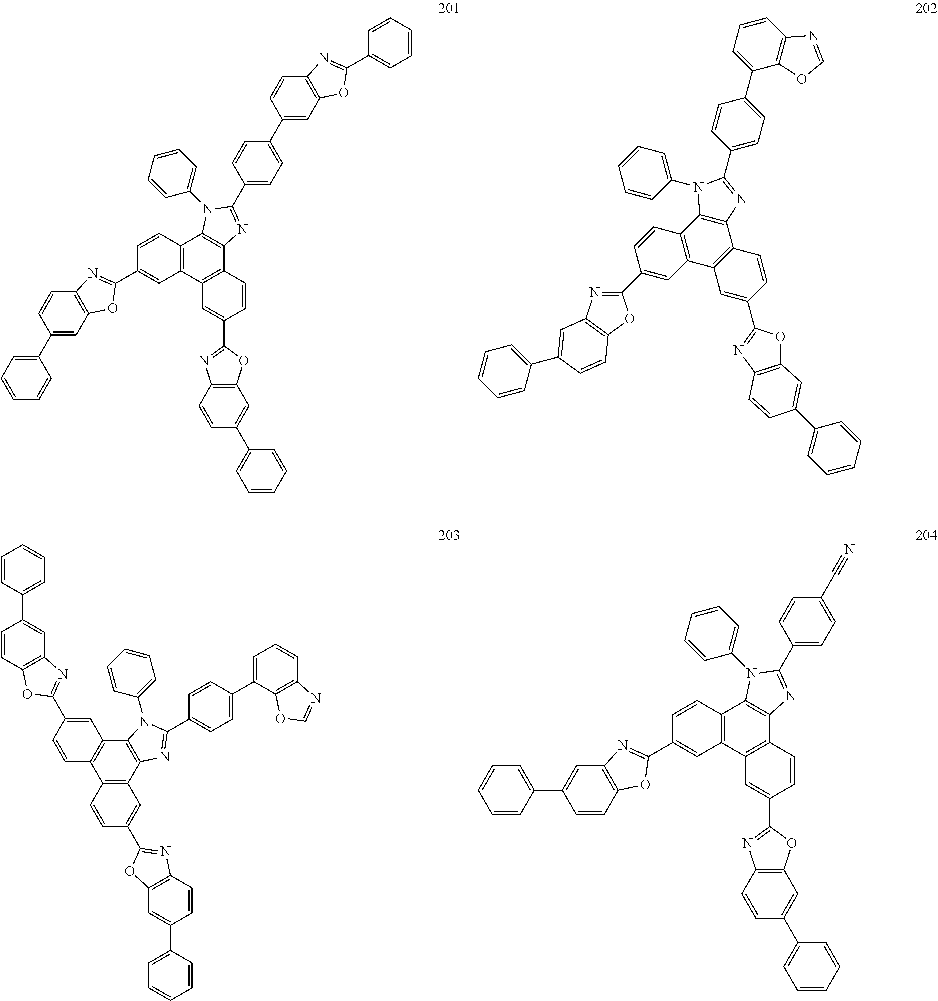 [SMILES: c1ccc(-c2ccc3nc(-c4ccc5c(c4)c4cc(-c6nc7ccc(-c8ccccc8)cc7o6)ccc4c4c5nc(-c5ccc(-c6ccc7nc(-c8ccccc8)oc7c6)cc5)n4-c4ccccc4)oc3c2)cc1 ; c1ccc(-c2ccc3oc(-c4ccc5c(c4)c4cc(-c6nc7ccc(-c8ccccc8)cc7o6)ccc4c4nc(-c6ccc(-c7cccc8ncoc78)cc6)n(-c6ccccc6)c54)nc3c2)cc1 ; c1ccc(-c2ccc3oc(-c4ccc5c6ccc(-c7nc8ccc(-c9ccccc9)cc8o7)cc6c6nc(-c7ccc(-c8cccc9ncoc89)cc7)n(-c7ccccc7)c6c5c4)nc3c2)cc1 ; N#Cc1ccc(-c2nc3c4ccc(-c5nc6ccc(-c7ccccc7)cc6o5)cc4c4cc(-c5nc6cc(-c7ccccc7)ccc6o5)ccc4c3n2-c2ccccc2)cc1]

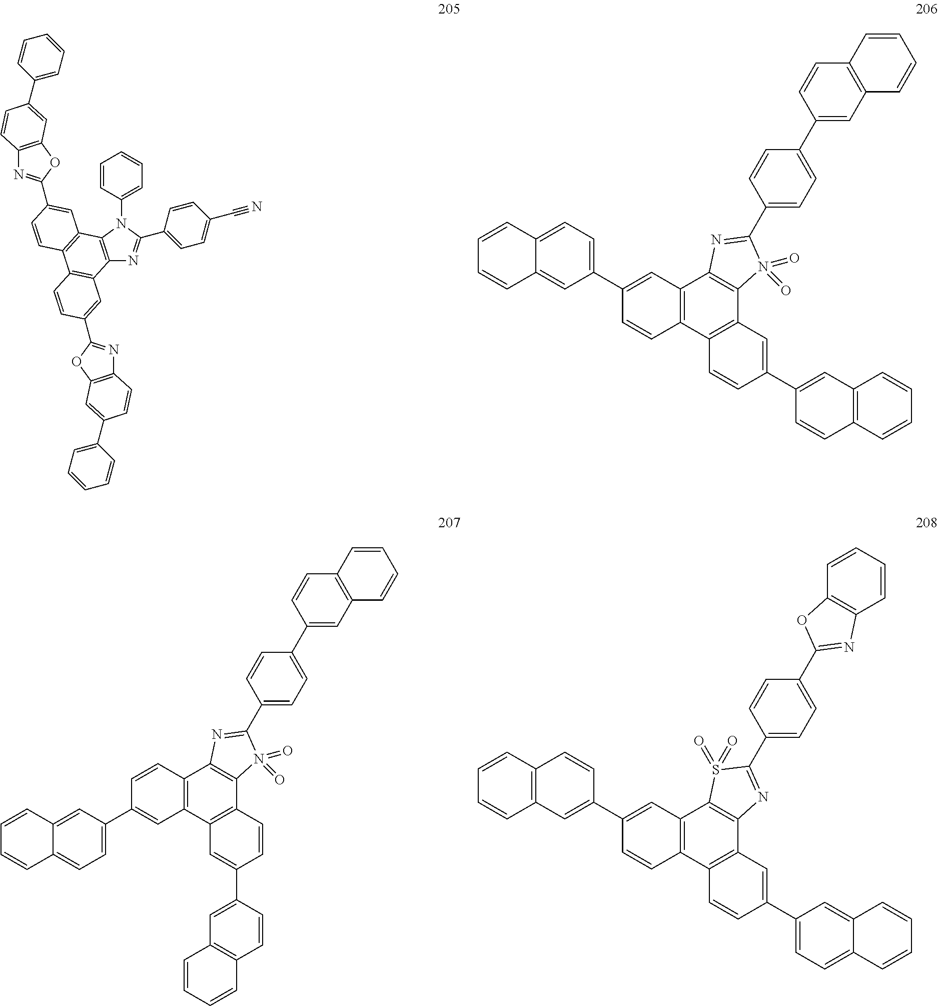

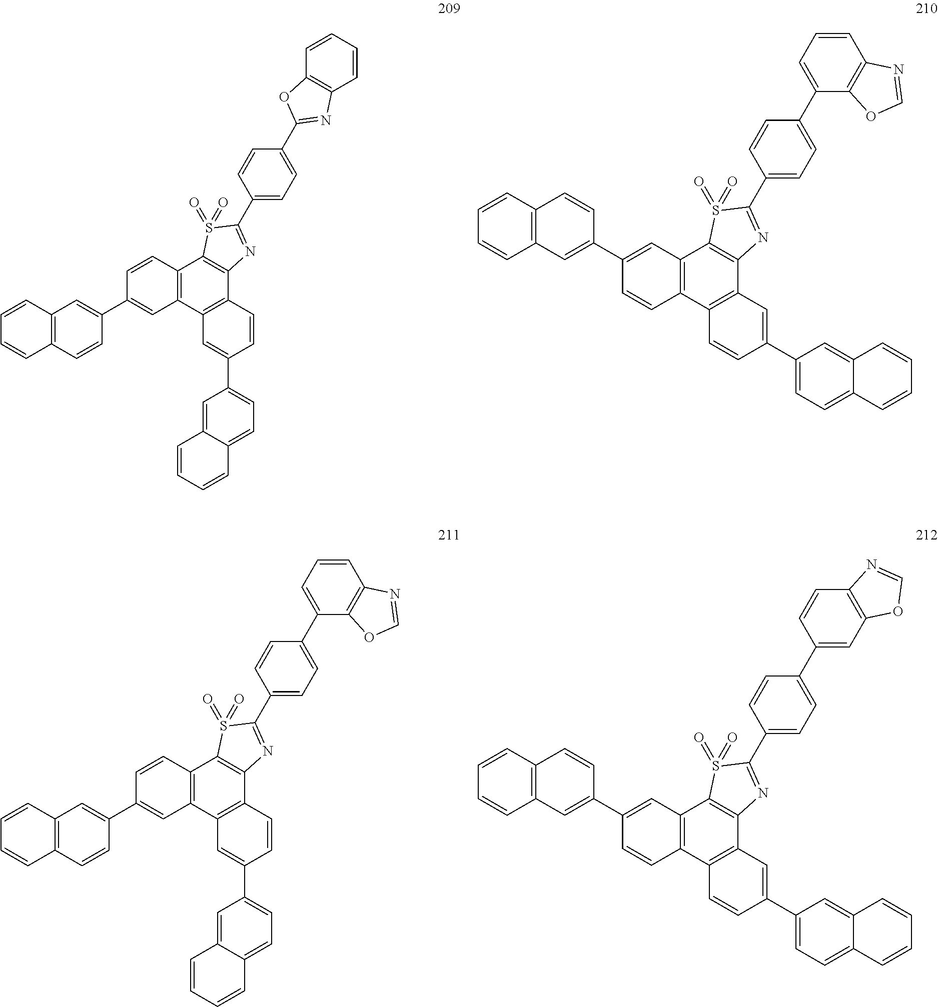 [SMILES: O=S1(=O)C(c2ccc(-c3nc4ccccc4o3)cc2)=Nc2c1c1ccc(-c3ccc4ccccc4c3)cc1c1cc(-c3ccc4ccccc4c3)ccc21 ; O=S1(=O)C(c2ccc(-c3cccc4ncoc34)cc2)=Nc2c1c1cc(-c3ccc4ccccc4c3)ccc1c1ccc(-c3ccc4ccccc4c3)cc21 ; O=S1(=O)C(c2ccc(-c3cccc4ncoc34)cc2)=Nc2c1c1ccc(-c3ccc4ccccc4c3)cc1c1cc(-c3ccc4ccccc4c3)ccc21 ; O=S1(=O)C(c2ccc(-c3ccc4ncoc4c3)cc2)=Nc2c1c1cc(-c3ccc4ccccc4c3)ccc1c1ccc(-c3ccc4ccccc4c3)cc21]

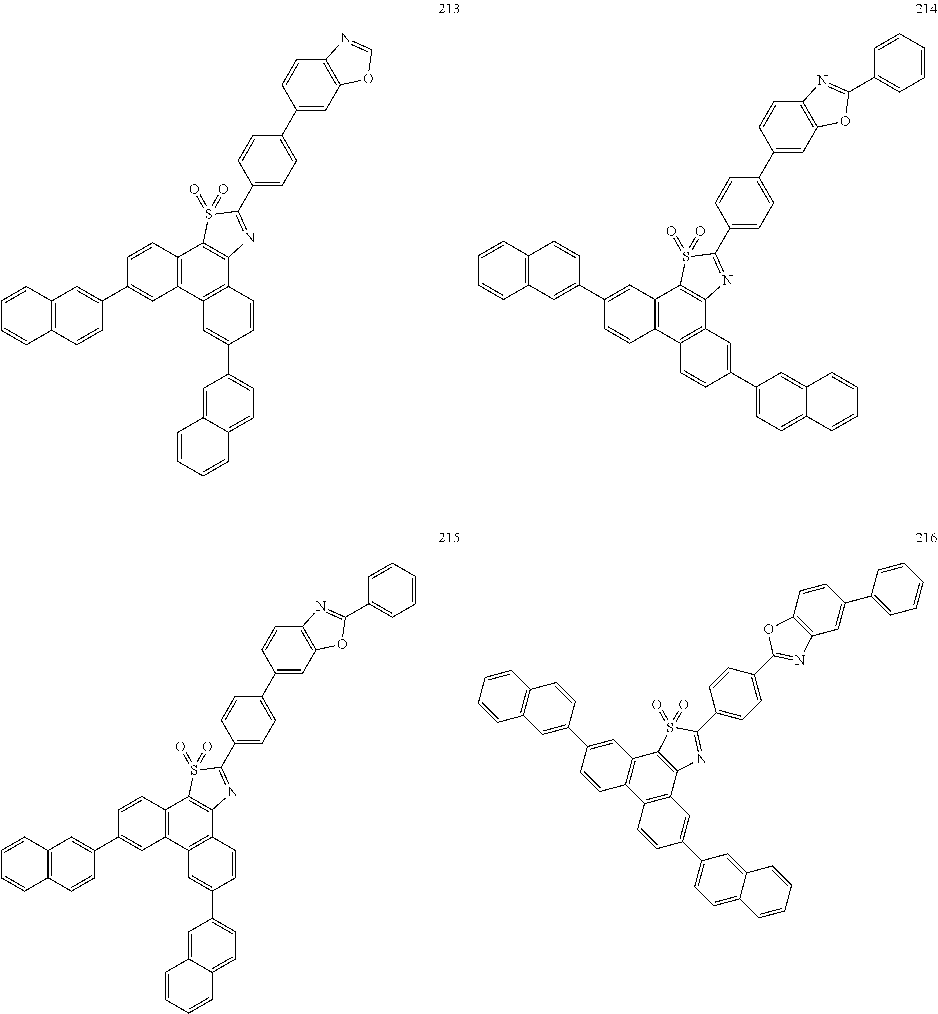 [SMILES: O=S1(=O)C(c2ccc(-c3ccc4ncoc4c3)cc2)=Nc2c1c1ccc(-c3ccc4ccccc4c3)cc1c1cc(-c3ccc4ccccc4c3)ccc21 ; O=S1(=O)C(c2ccc(-c3ccc4nc(-c5ccccc5)oc4c3)cc2)=Nc2c1c1cc(-c3ccc4ccccc4c3)ccc1c1ccc(-c3ccc4ccccc4c3)cc21 ; O=S1(=O)C(c2ccc(-c3ccc4nc(-c5ccccc5)oc4c3)cc2)=Nc2c1c1ccc(-c3ccc4ccccc4c3)cc1c1cc(-c3ccc4ccccc4c3)ccc21 ; O=S1(=O)C(c2ccc(-c3nc4cc(-c5ccccc5)ccc4o3)cc2)=Nc2c1c1cc(-c3ccc4ccccc4c3)ccc1c1ccc(-c3ccc4ccccc4c3)cc21]

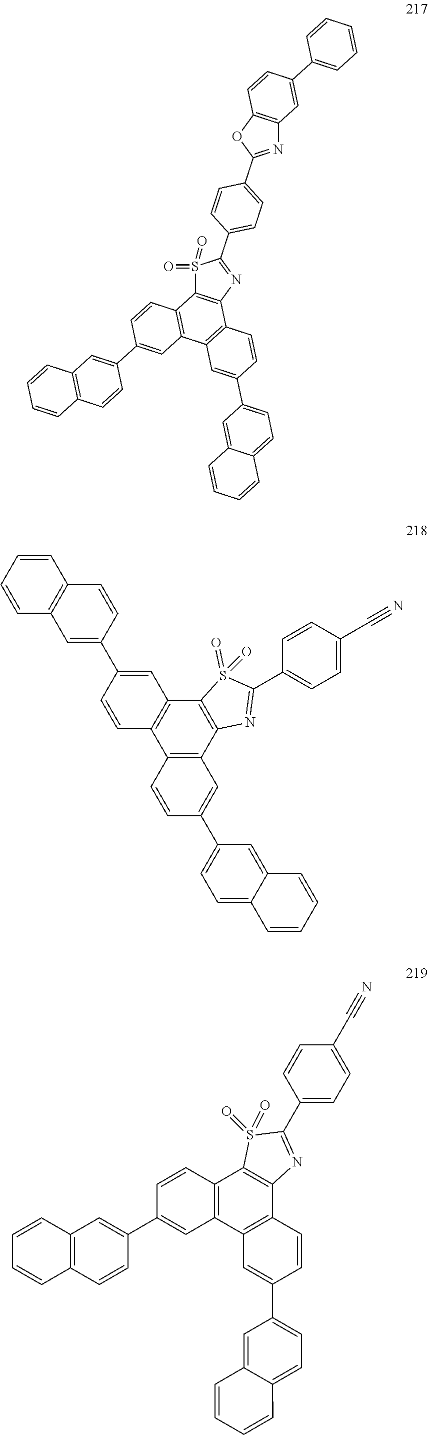 [SMILES: O=S1(=O)C(c2ccc(-c3nc4cc(-c5ccccc5)ccc4o3)cc2)=Nc2c1c1ccc(-c3ccc4ccccc4c3)cc1c1cc(-c3ccc4ccccc4c3)ccc21 ; N#Cc1ccc(C2=Nc3c(c4cc(-c5ccc6ccccc6c5)ccc4c4ccc(-c5ccc6ccccc6c5)cc34)S2(=O)=O)cc1 ; N#Cc1ccc(C2=Nc3c(c4ccc(-c5ccc6ccccc6c5)cc4c4cc(-c5ccc6ccccc6c5)ccc34)S2(=O)=O)cc1]

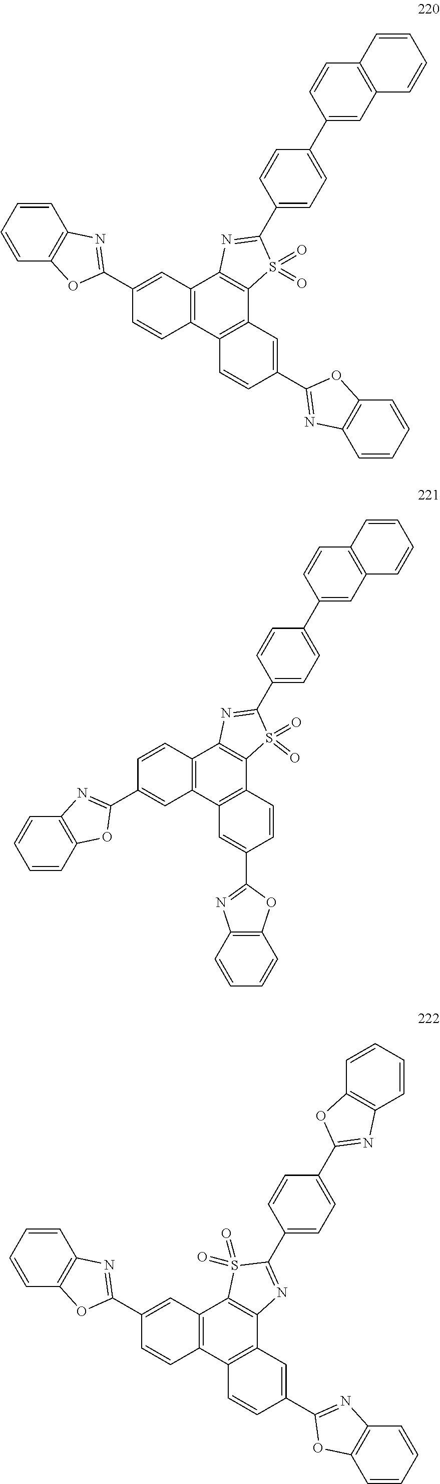 [SMILES: O=S1(=O)C(c2ccc(-c3ccc4ccccc4c3)cc2)=Nc2c1c1cc(-c3nc4ccccc4o3)ccc1c1ccc(-c3nc4ccccc4o3)cc21 ; O=S1(=O)C(c2ccc(-c3ccc4ccccc4c3)cc2)=Nc2c1c1ccc(-c3nc4ccccc4o3)cc1c1cc(-c3nc4ccccc4o3)ccc21 ; O=S1(=O)C(c2ccc(-c3nc4ccccc4o3)cc2)=Nc2c1c1cc(-c3nc4ccccc4o3)ccc1c1ccc(-c3nc4ccccc4o3)cc21]

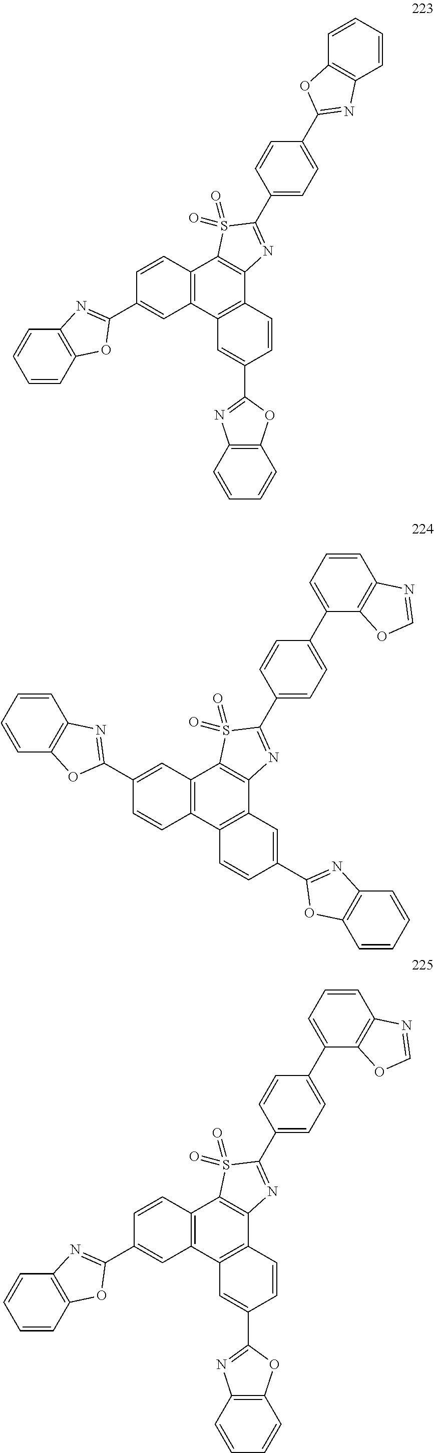

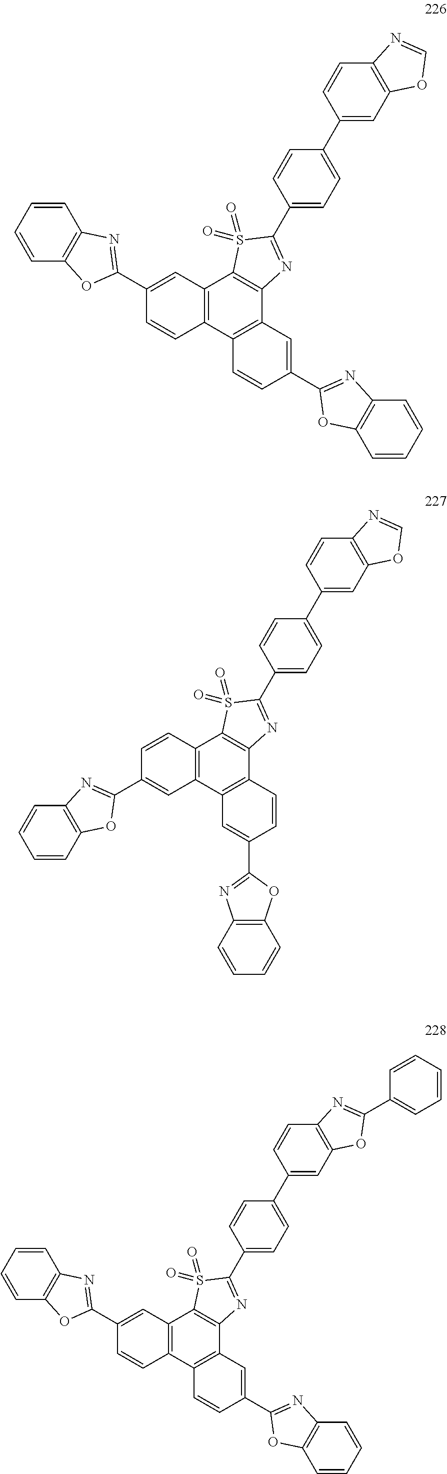 [SMILES: O=S1(=O)C(c2ccc(-c3ccc4ncoc4c3)cc2)=Nc2c1c1cc(-c3nc4ccccc4o3)ccc1c1ccc(-c3nc4ccccc4o3)cc21 ; O=S1(=O)C(c2ccc(-c3ccc4ncoc4c3)cc2)=Nc2c1c1ccc(-c3nc4ccccc4o3)cc1c1cc(-c3nc4ccccc4o3)ccc21 ; O=S1(=O)C(c2ccc(-c3ccc4nc(-c5ccccc5)oc4c3)cc2)=Nc2c1c1cc(-c3nc4ccccc4o3)ccc1c1ccc(-c3nc4ccccc4o3)cc21]

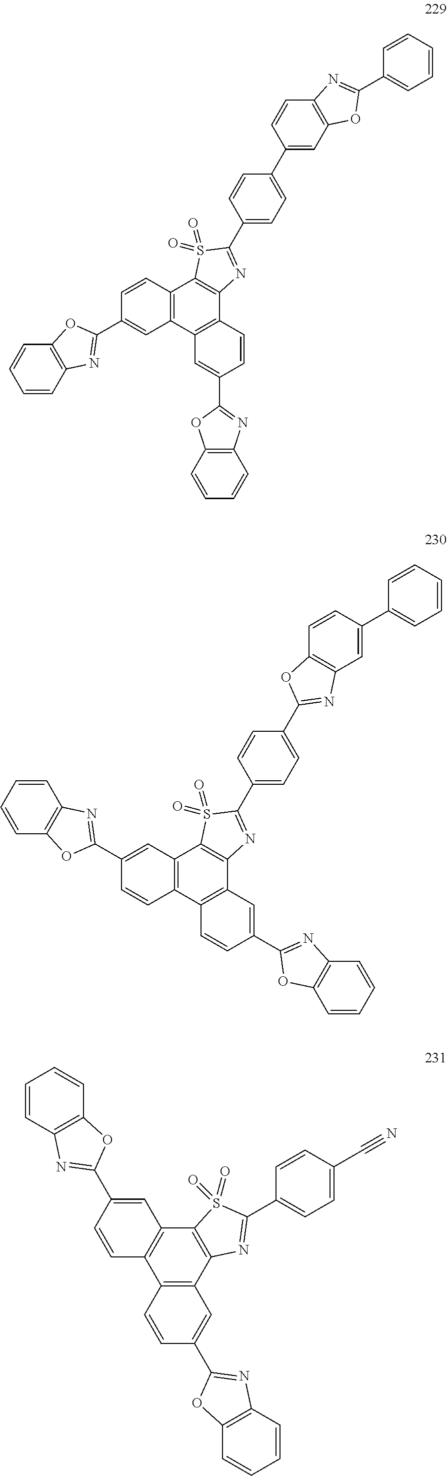 [SMILES: O=S1(=O)C(c2ccc(-c3ccc4nc(-c5ccccc5)oc4c3)cc2)=Nc2c1c1ccc(-c3nc4ccccc4o3)cc1c1cc(-c3nc4ccccc4o3)ccc21 ; O=S1(=O)C(c2ccc(-c3nc4cc(-c5ccccc5)ccc4o3)cc2)=Nc2c1c1cc(-c3nc4ccccc4o3)ccc1c1ccc(-c3nc4ccccc4o3)cc21 ; N#Cc1ccc(C2=Nc3c(c4cc(-c5nc6ccccc6o5)ccc4c4ccc(-c5nc6ccccc6o5)cc34)S2(=O)=O)cc1]

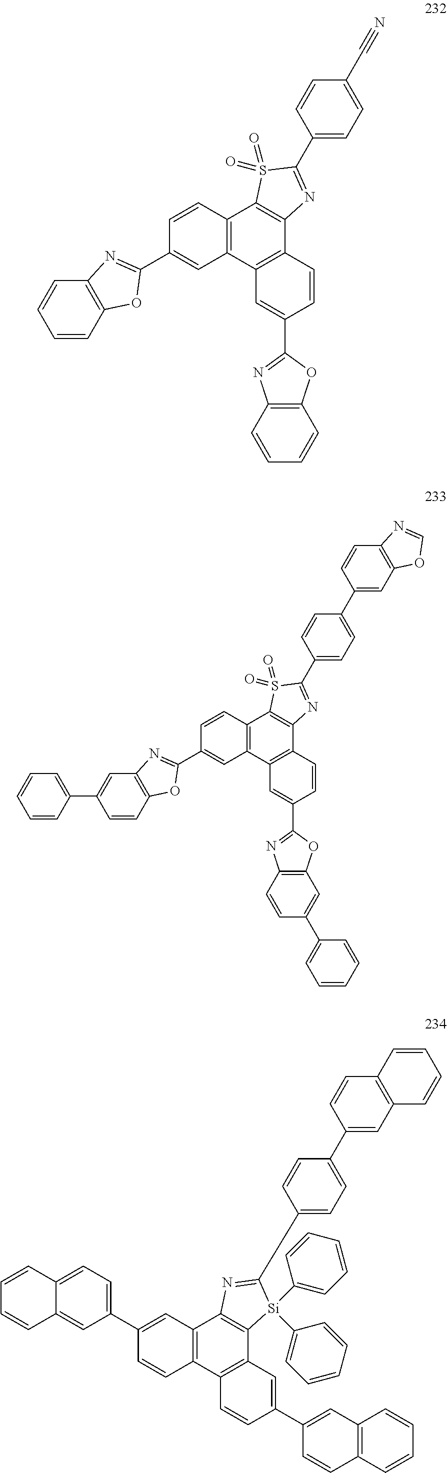 [SMILES: N#Cc1ccc(C2=Nc3c(c4ccc(-c5nc6ccccc6o5)cc4c4cc(-c5nc6ccccc6o5)ccc34)S2(=O)=O)cc1 ; O=S1(=O)C(c2ccc(-c3ccc4ncoc4c3)cc2)=Nc2c1c1ccc(-c3nc4cc(-c5ccccc5)ccc4o3)cc1c1cc(-c3nc4ccc(-c5ccccc5)cc4o3)ccc21 ; c1ccc([Si]2(c3ccccc3)C(c3ccc(-c4ccc5ccccc5c4)cc3)=Nc3c2c2cc(-c4ccc5ccccc5c4)ccc2c2ccc(-c4ccc5ccccc5c4)cc32)cc1]

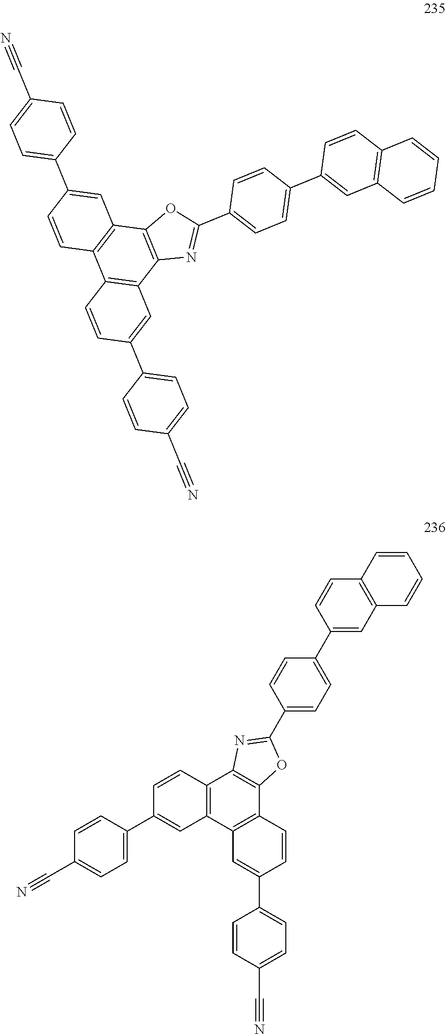 [SMILES: N#Cc1ccc(-c2ccc3c4ccc(-c5ccc(C#N)cc5)cc4c4oc(-c5ccc(-c6ccc7ccccc7c6)cc5)nc4c3c2)cc1 ; N#Cc1ccc(-c2ccc3c(c2)c2cc(-c4ccc(C#N)cc4)ccc2c2oc(-c4ccc(-c5ccc6ccccc6c5)cc4)nc32)cc1]

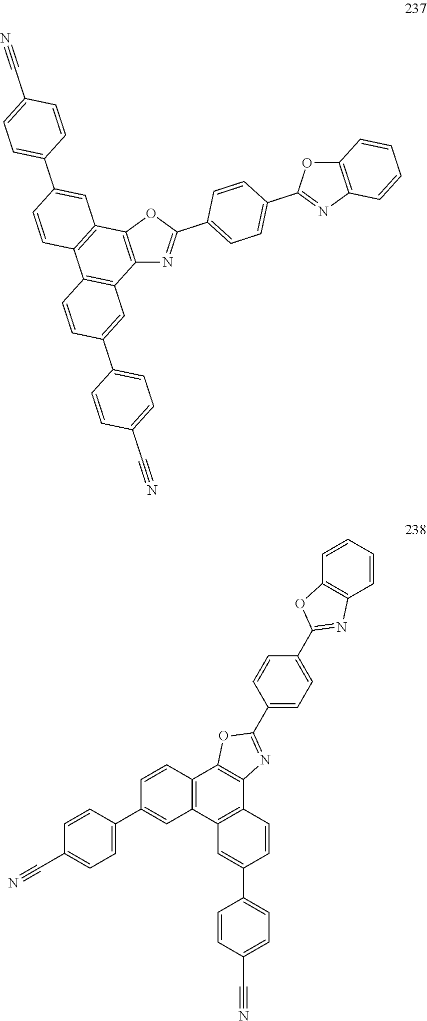 [SMILES: N#Cc1ccc(-c2ccc3c4ccc(-c5ccc(C#N)cc5)cc4c4oc(-c5ccc(-c6nc7ccccc7o6)cc5)nc4c3c2)cc1 ; N#Cc1ccc(-c2ccc3c(c2)c2cc(-c4ccc(C#N)cc4)ccc2c2oc(-c4ccc(-c5nc6ccccc6o5)cc4)nc32)cc1]

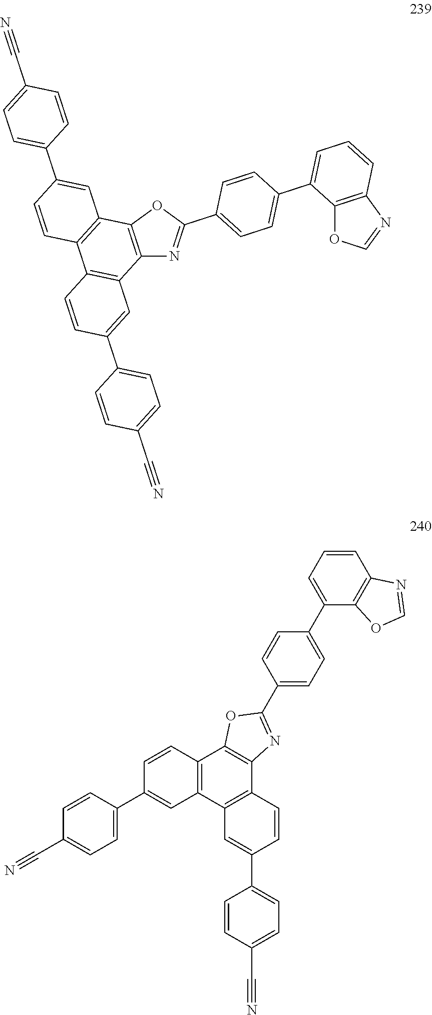 [SMILES: N#Cc1ccc(-c2ccc3c4ccc(-c5ccc(C#N)cc5)cc4c4oc(-c5ccc(-c6cccc7ncoc67)cc5)nc4c3c2)cc1 ; N#Cc1ccc(-c2ccc3c(c2)c2cc(-c4ccc(C#N)cc4)ccc2c2oc(-c4ccc(-c5cccc6ncoc56)cc4)nc32)cc1]

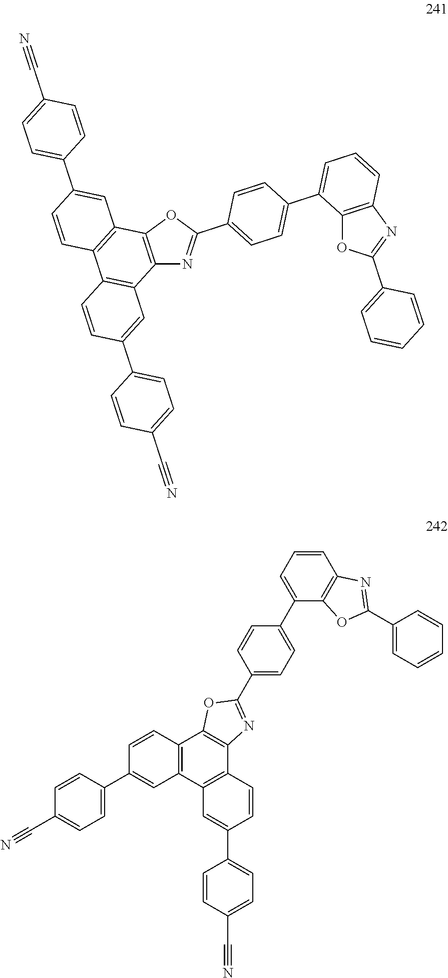 [SMILES: N#Cc1ccc(-c2ccc3c4ccc(-c5ccc(C#N)cc5)cc4c4oc(-c5ccc(-c6cccc7nc(-c8ccccc8)oc67)cc5)nc4c3c2)cc1 ; N#Cc1ccc(-c2ccc3c(c2)c2cc(-c4ccc(C#N)cc4)ccc2c2oc(-c4ccc(-c5cccc6nc(-c7ccccc7)oc56)cc4)nc32)cc1]

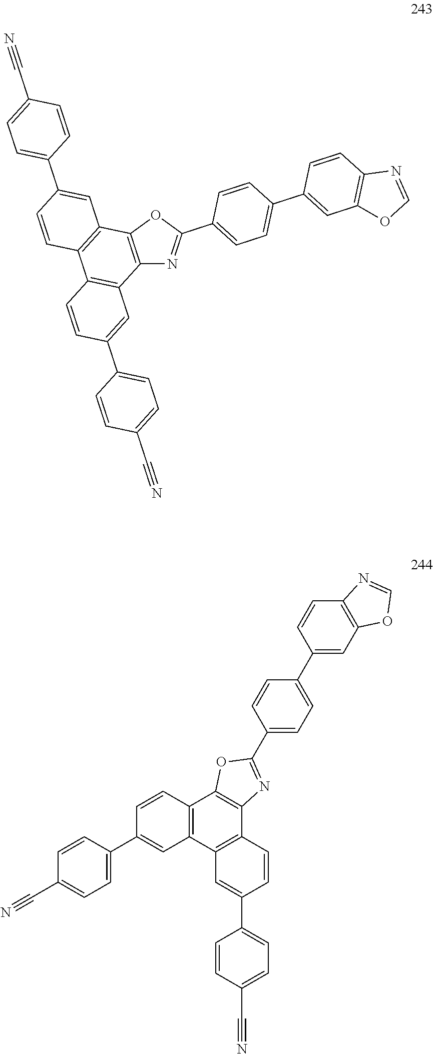 [SMILES: N#Cc1ccc(-c2ccc3c4ccc(-c5ccc(C#N)cc5)cc4c4oc(-c5ccc(-c6ccc7ncoc7c6)cc5)nc4c3c2)cc1 ; N#Cc1ccc(-c2ccc3c(c2)c2cc(-c4ccc(C#N)cc4)ccc2c2oc(-c4ccc(-c5ccc6ncoc6c5)cc4)nc32)cc1]

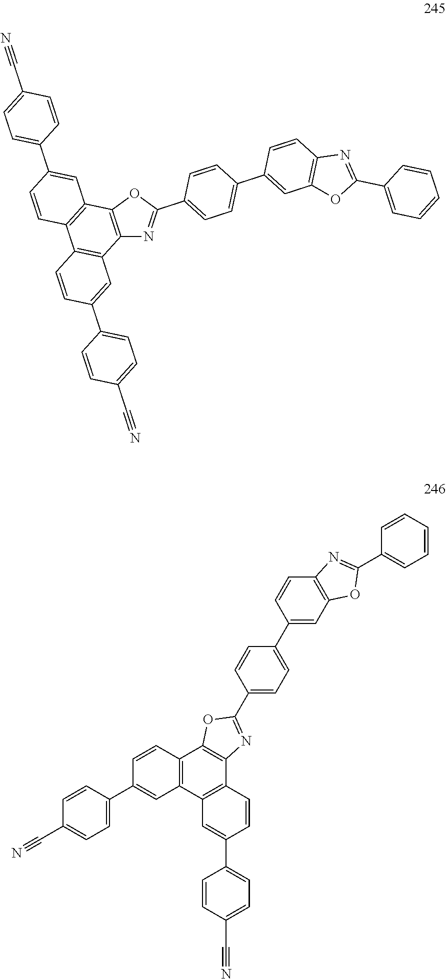 [SMILES: N#Cc1ccc(-c2ccc3c4ccc(-c5ccc(C#N)cc5)cc4c4oc(-c5ccc(-c6ccc7nc(-c8ccccc8)oc7c6)cc5)nc4c3c2)cc1 ; N#Cc1ccc(-c2ccc3c(c2)c2cc(-c4ccc(C#N)cc4)ccc2c2oc(-c4ccc(-c5ccc6nc(-c7ccccc7)oc6c5)cc4)nc32)cc1]

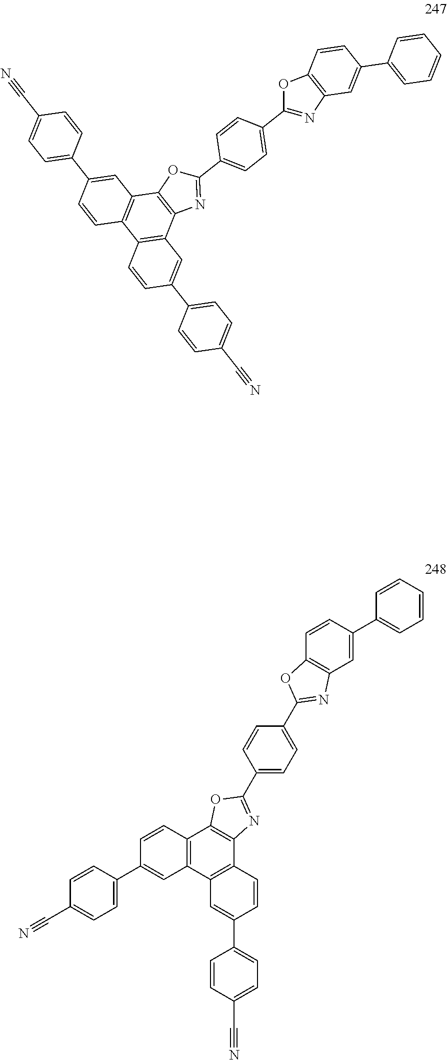 [SMILES: N#Cc1ccc(-c2ccc3c4ccc(-c5ccc(C#N)cc5)cc4c4oc(-c5ccc(-c6nc7cc(-c8ccccc8)ccc7o6)cc5)nc4c3c2)cc1 ; N#Cc1ccc(-c2ccc3c(c2)c2cc(-c4ccc(C#N)cc4)ccc2c2oc(-c4ccc(-c5nc6cc(-c7ccccc7)ccc6o5)cc4)nc32)cc1]

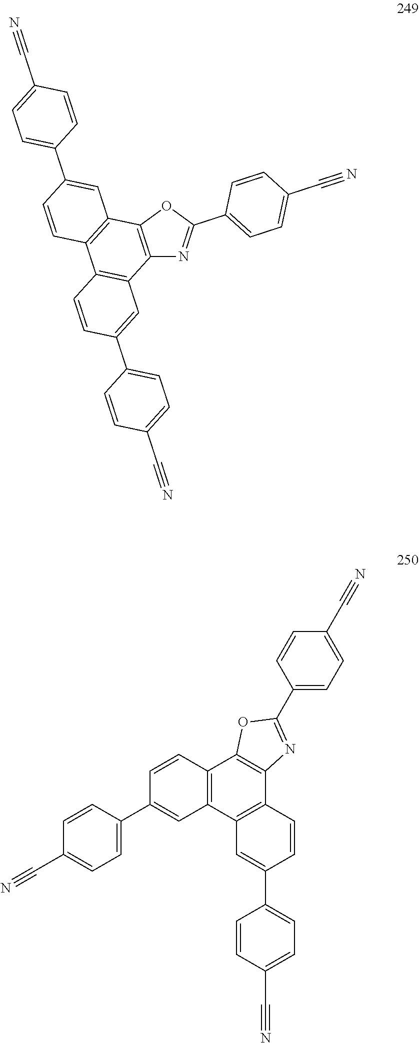 [SMILES: N#Cc1ccc(-c2ccc3c4ccc(-c5ccc(C#N)cc5)cc4c4oc(-c5ccc(C#N)cc5)nc4c3c2)cc1 ; N#Cc1ccc(-c2ccc3c(c2)c2cc(-c4ccc(C#N)cc4)ccc2c2oc(-c4ccc(C#N)cc4)nc32)cc1]

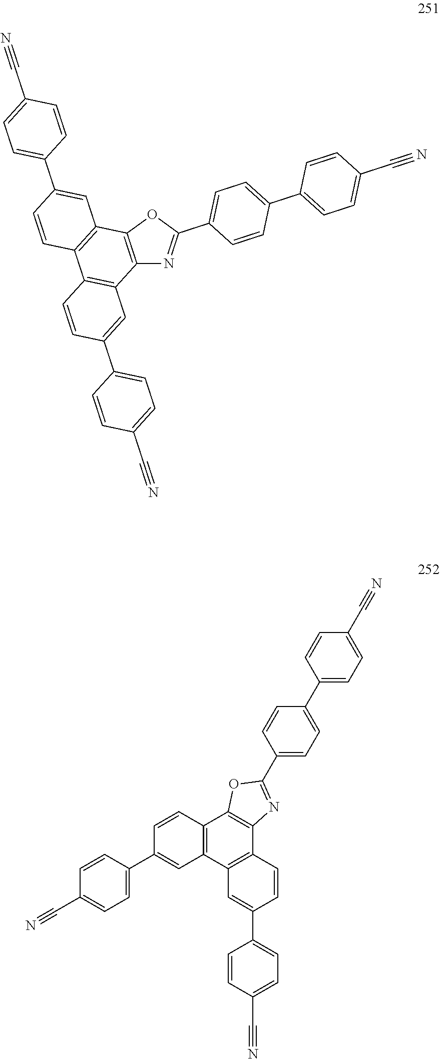 [SMILES: N#Cc1ccc(-c2ccc(-c3nc4c5cc(-c6ccc(C#N)cc6)ccc5c5ccc(-c6ccc(C#N)cc6)cc5c4o3)cc2)cc1 ; N#Cc1ccc(-c2ccc(-c3nc4c5ccc(-c6ccc(C#N)cc6)cc5c5cc(-c6ccc(C#N)cc6)ccc5c4o3)cc2)cc1]

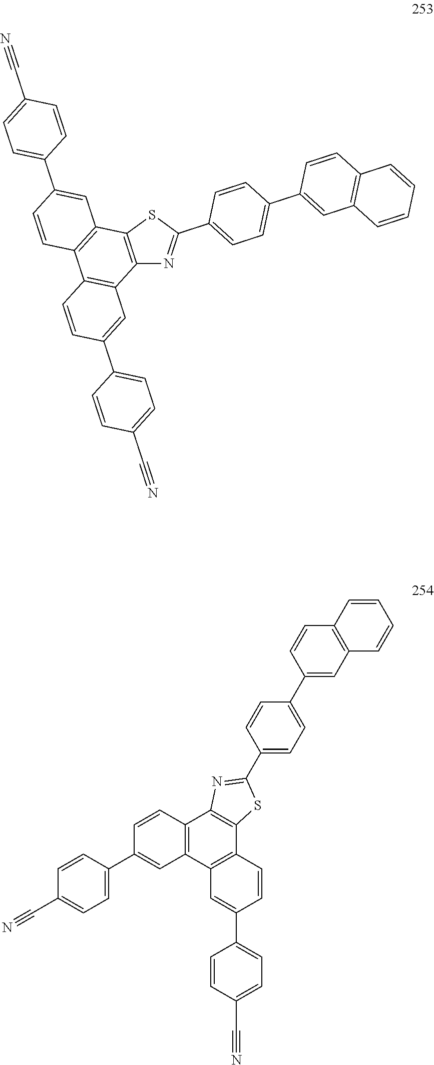 [SMILES: N#Cc1ccc(-c2ccc3c4ccc(-c5ccc(C#N)cc5)cc4c4sc(-c5ccc(-c6ccc7ccccc7c6)cc5)nc4c3c2)cc1 ; N#Cc1ccc(-c2ccc3c(c2)c2cc(-c4ccc(C#N)cc4)ccc2c2sc(-c4ccc(-c5ccc6ccccc6c5)cc4)nc32)cc1]

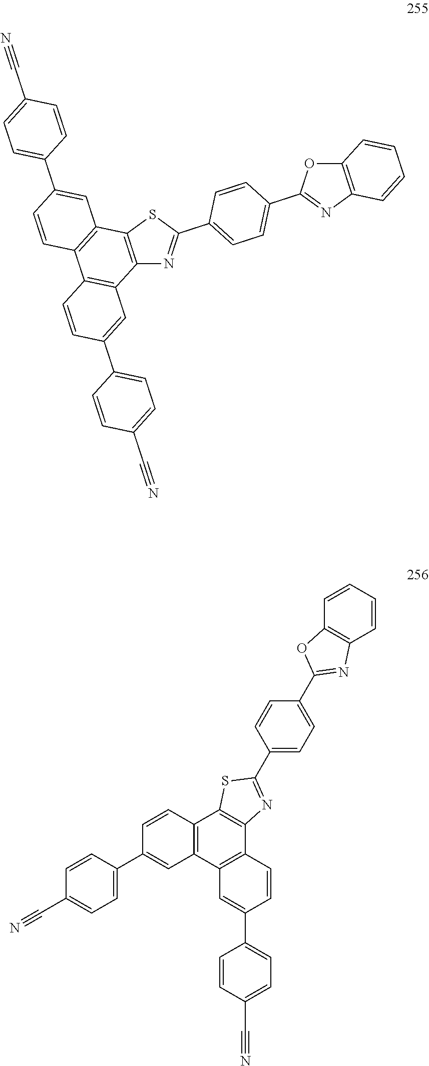 [SMILES: N#Cc1ccc(-c2ccc3c4ccc(-c5ccc(C#N)cc5)cc4c4sc(-c5ccc(-c6nc7ccccc7o6)cc5)nc4c3c2)cc1 ; N#Cc1ccc(-c2ccc3c(c2)c2cc(-c4ccc(C#N)cc4)ccc2c2sc(-c4ccc(-c5nc6ccccc6o5)cc4)nc32)cc1]

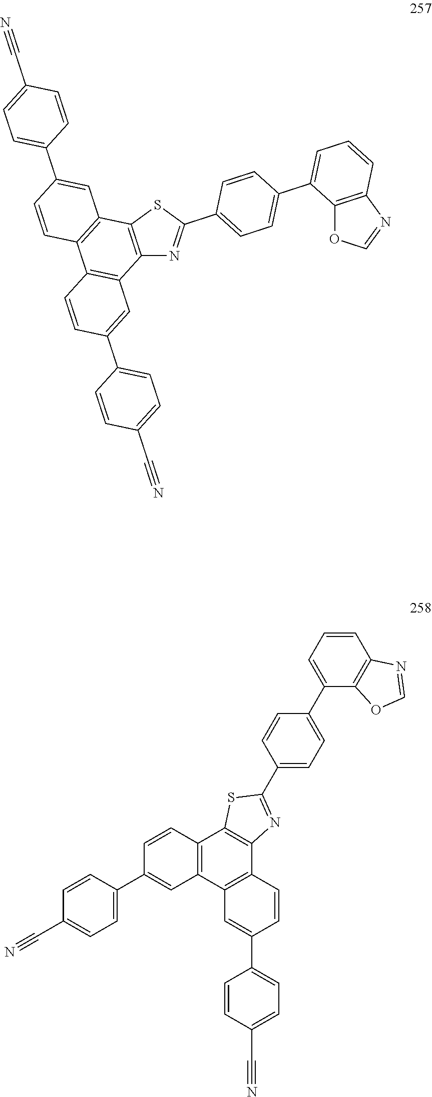 [SMILES: N#Cc1ccc(-c2ccc3c4ccc(-c5ccc(C#N)cc5)cc4c4sc(-c5ccc(-c6cccc7ncoc67)cc5)nc4c3c2)cc1 ; N#Cc1ccc(-c2ccc3c(c2)c2cc(-c4ccc(C#N)cc4)ccc2c2sc(-c4ccc(-c5cccc6ncoc56)cc4)nc32)cc1]

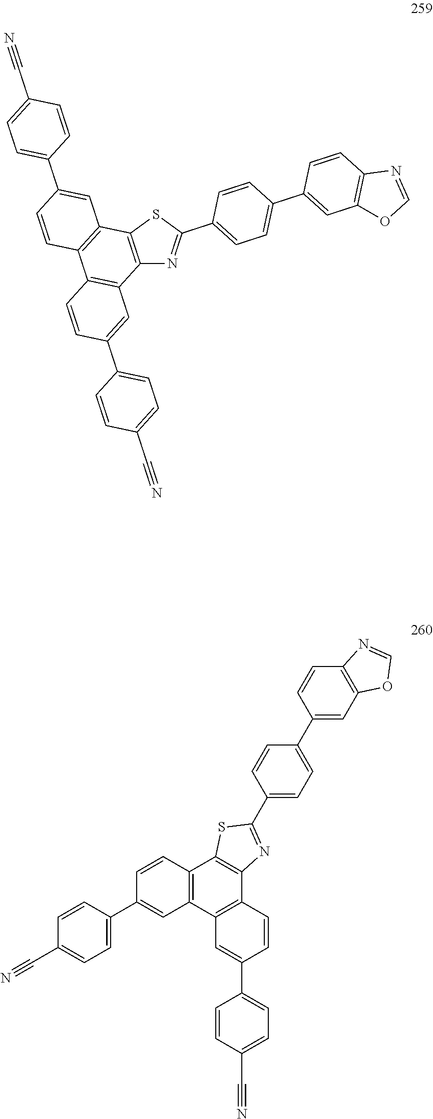 [SMILES: N#Cc1ccc(-c2ccc3c4ccc(-c5ccc(C#N)cc5)cc4c4sc(-c5ccc(-c6ccc7ncoc7c6)cc5)nc4c3c2)cc1 ; N#Cc1ccc(-c2ccc3c(c2)c2cc(-c4ccc(C#N)cc4)ccc2c2sc(-c4ccc(-c5ccc6ncoc6c5)cc4)nc32)cc1]

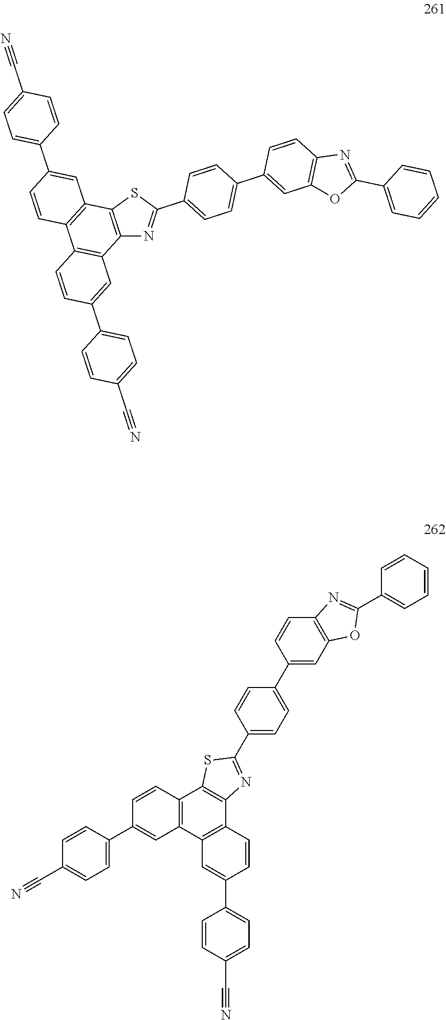 [SMILES: N#Cc1ccc(-c2ccc3c4ccc(-c5ccc(C#N)cc5)cc4c4sc(-c5ccc(-c6ccc7nc(-c8ccccc8)oc7c6)cc5)nc4c3c2)cc1 ; N#Cc1ccc(-c2ccc3c(c2)c2cc(-c4ccc(C#N)cc4)ccc2c2sc(-c4ccc(-c5ccc6nc(-c7ccccc7)oc6c5)cc4)nc32)cc1]

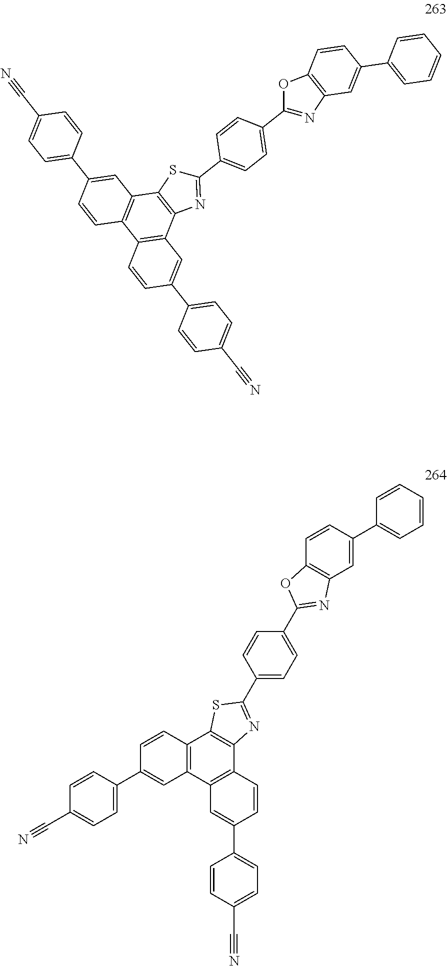 [SMILES: N#Cc1ccc(-c2ccc3c4ccc(-c5ccc(C#N)cc5)cc4c4sc(-c5ccc(-c6nc7cc(-c8ccccc8)ccc7o6)cc5)nc4c3c2)cc1 ; N#Cc1ccc(-c2ccc3c(c2)c2cc(-c4ccc(C#N)cc4)ccc2c2sc(-c4ccc(-c5nc6cc(-c7ccccc7)ccc6o5)cc4)nc32)cc1]

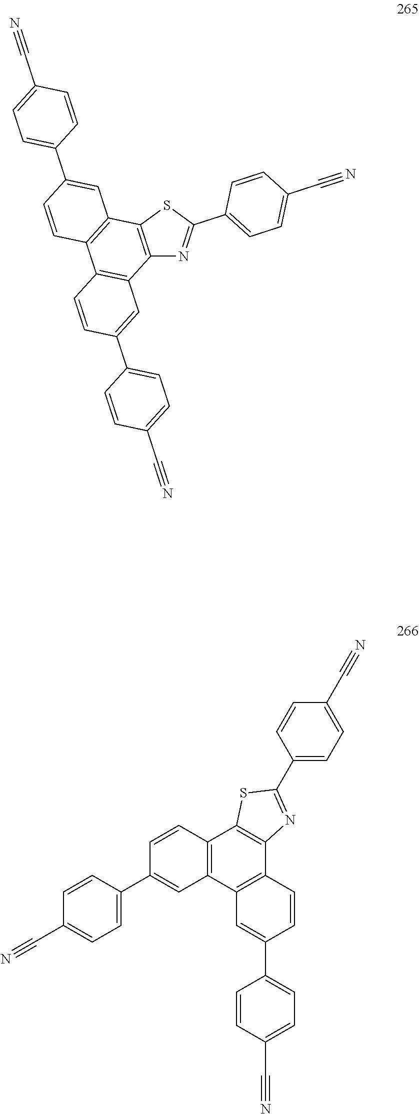 [SMILES: N#Cc1ccc(-c2ccc3c4ccc(-c5ccc(C#N)cc5)cc4c4sc(-c5ccc(C#N)cc5)nc4c3c2)cc1 ; N#Cc1ccc(-c2ccc3c(c2)c2cc(-c4ccc(C#N)cc4)ccc2c2sc(-c4ccc(C#N)cc4)nc32)cc1]

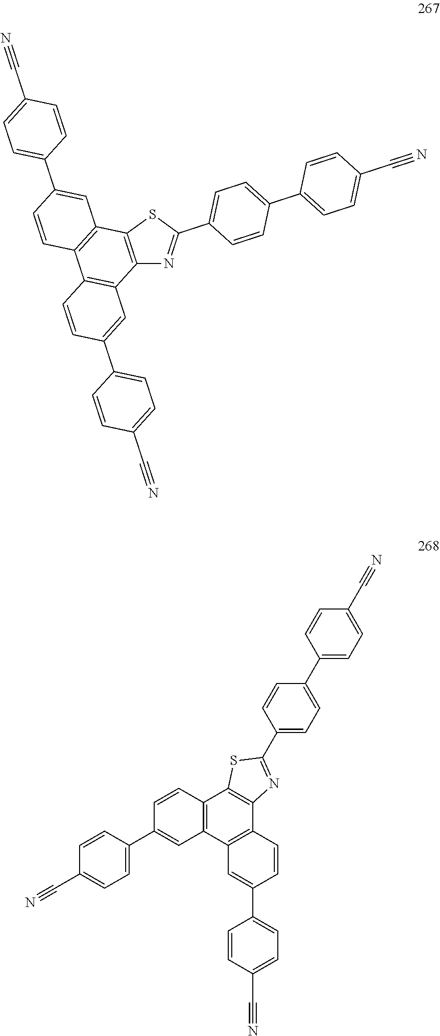 [SMILES: N#Cc1ccc(-c2ccc(-c3nc4c5cc(-c6ccc(C#N)cc6)ccc5c5ccc(-c6ccc(C#N)cc6)cc5c4s3)cc2)cc1 ; N#Cc1ccc(-c2ccc(-c3nc4c5ccc(-c6ccc(C#N)cc6)cc5c5cc(-c6ccc(C#N)cc6)ccc5c4s3)cc2)cc1]

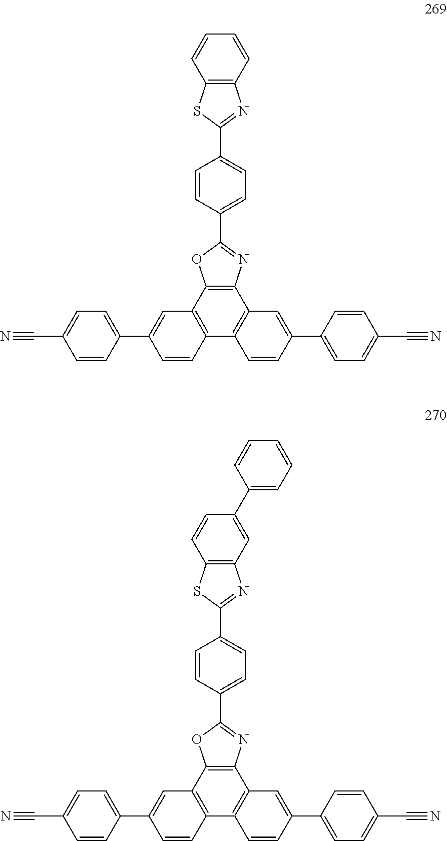 [SMILES: N#Cc1ccc(-c2ccc3c4ccc(-c5ccc(C#N)cc5)cc4c4oc(-c5ccc(-c6nc7ccccc7s6)cc5)nc4c3c2)cc1 ; N#Cc1ccc(-c2ccc3c4ccc(-c5ccc(C#N)cc5)cc4c4oc(-c5ccc(-c6nc7cc(-c8ccccc8)ccc7s6)cc5)nc4c3c2)cc1]

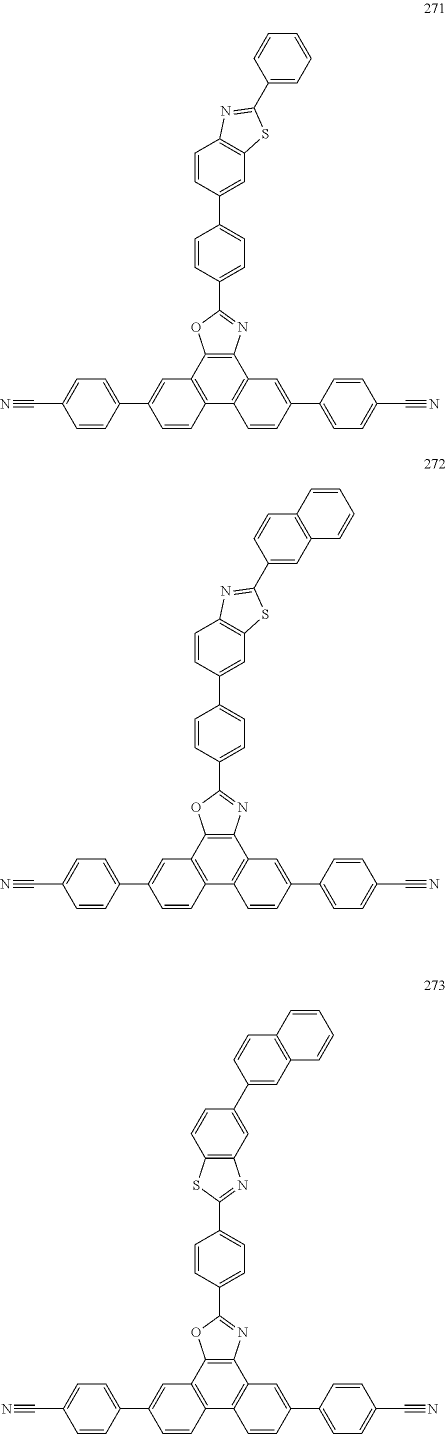 [SMILES: N#Cc1ccc(-c2ccc3c4ccc(-c5ccc(C#N)cc5)cc4c4oc(-c5ccc(-c6ccc7nc(-c8ccccc8)sc7c6)cc5)nc4c3c2)cc1 ; N#Cc1ccc(-c2ccc3c4ccc(-c5ccc(C#N)cc5)cc4c4oc(-c5ccc(-c6ccc7nc(-c8ccc9ccccc9c8)sc7c6)cc5)nc4c3c2)cc1 ; N#Cc1ccc(-c2ccc3c4ccc(-c5ccc(C#N)cc5)cc4c4oc(-c5ccc(-c6nc7cc(-c8ccc9ccccc9c8)ccc7s6)cc5)nc4c3c2)cc1]

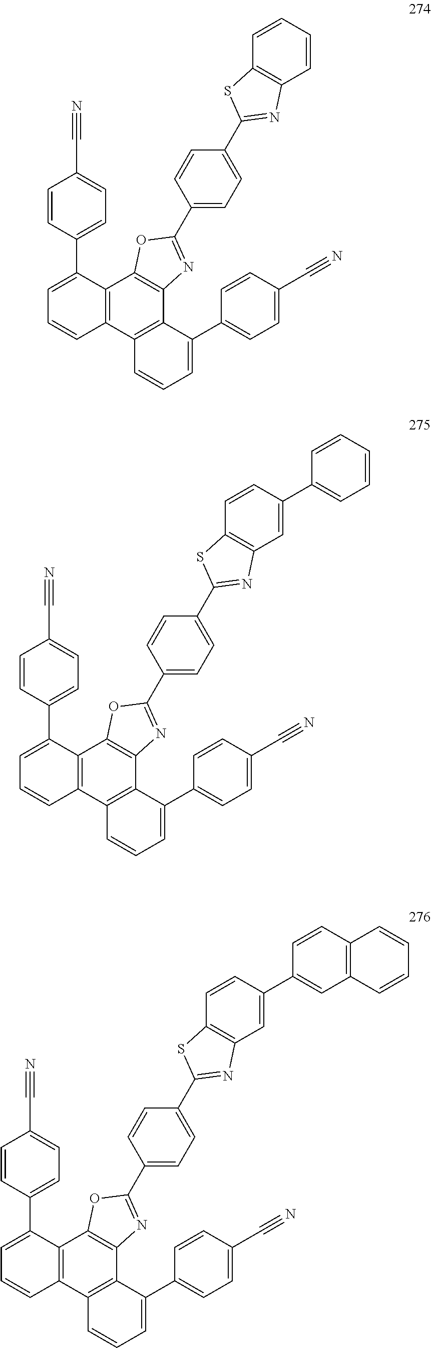 [SMILES: N#Cc1ccc(-c2cccc3c4cccc(-c5ccc(C#N)cc5)c4c4oc(-c5ccc(-c6nc7ccccc7s6)cc5)nc4c23)cc1 ; N#Cc1ccc(-c2cccc3c4cccc(-c5ccc(C#N)cc5)c4c4oc(-c5ccc(-c6nc7cc(-c8ccccc8)ccc7s6)cc5)nc4c23)cc1 ; N#Cc1ccc(-c2cccc3c4cccc(-c5ccc(C#N)cc5)c4c4oc(-c5ccc(-c6nc7cc(-c8ccc9ccccc9c8)ccc7s6)cc5)nc4c23)cc1]

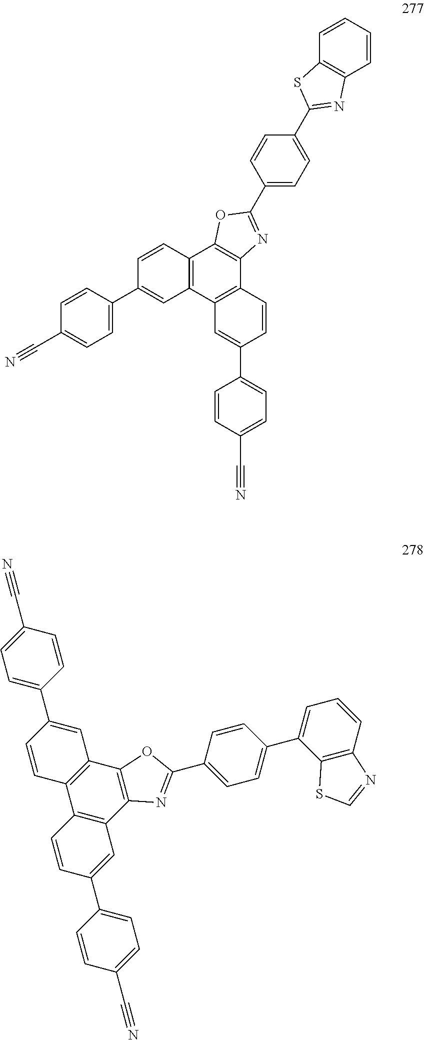 [SMILES: N#Cc1ccc(-c2ccc3c(c2)c2cc(-c4ccc(C#N)cc4)ccc2c2oc(-c4ccc(-c5nc6ccccc6s5)cc4)nc32)cc1 ; N#Cc1ccc(-c2ccc3c4ccc(-c5ccc(C#N)cc5)cc4c4oc(-c5ccc(-c6cccc7ncsc67)cc5)nc4c3c2)cc1]

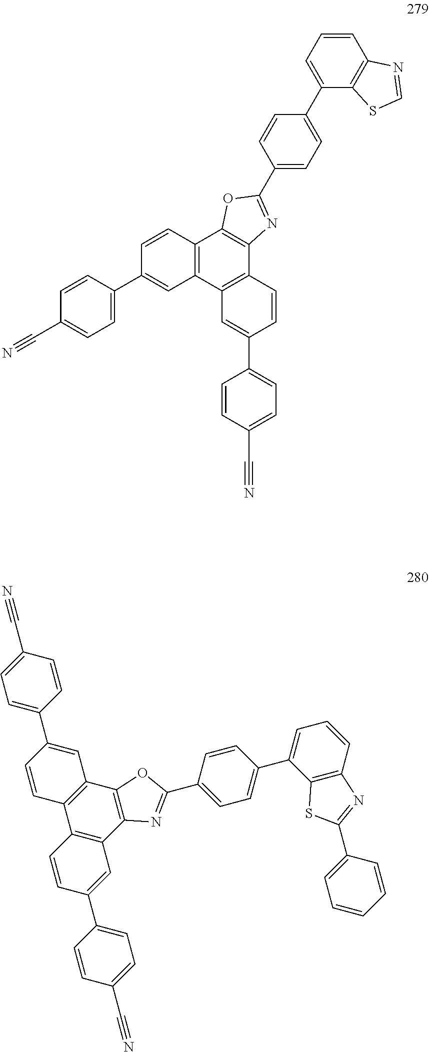 [SMILES: N#Cc1ccc(-c2ccc3c(c2)c2cc(-c4ccc(C#N)cc4)ccc2c2oc(-c4ccc(-c5cccc6ncsc56)cc4)nc32)cc1 ; N#Cc1ccc(-c2ccc3c4ccc(-c5ccc(C#N)cc5)cc4c4oc(-c5ccc(-c6cccc7nc(-c8ccccc8)sc67)cc5)nc4c3c2)cc1]

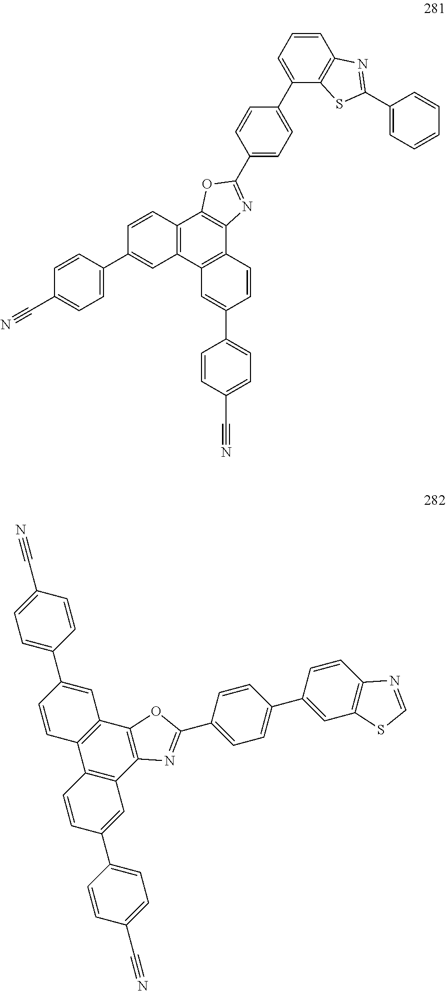 [SMILES: N#Cc1ccc(-c2ccc3c(c2)c2cc(-c4ccc(C#N)cc4)ccc2c2oc(-c4ccc(-c5cccc6nc(-c7ccccc7)sc56)cc4)nc32)cc1 ; N#Cc1ccc(-c2ccc3c4ccc(-c5ccc(C#N)cc5)cc4c4oc(-c5ccc(-c6ccc7ncsc7c6)cc5)nc4c3c2)cc1]

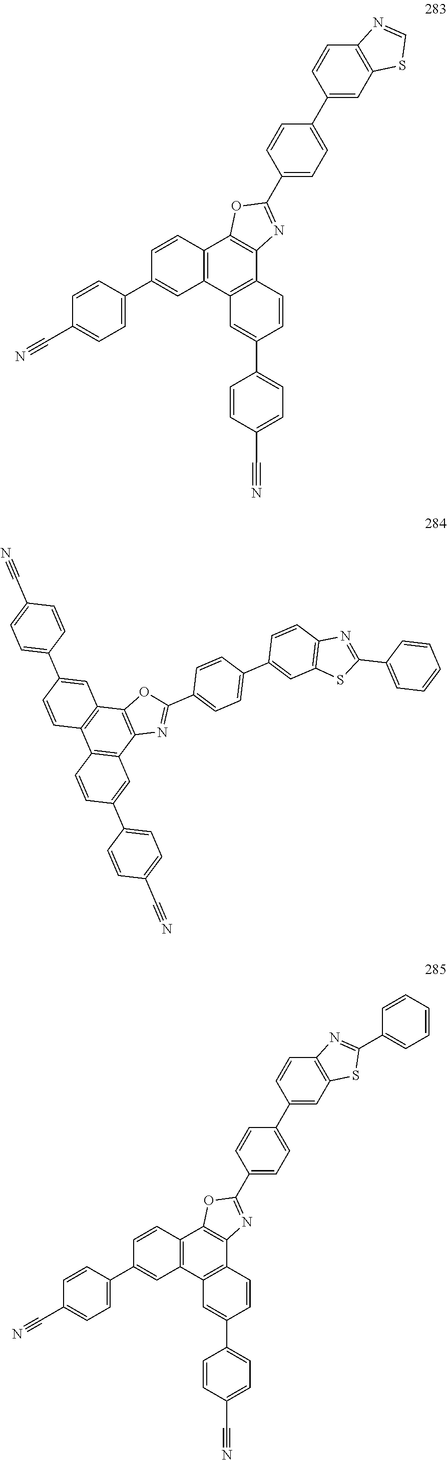 [SMILES: N#Cc1ccc(-c2ccc3c(c2)c2cc(-c4ccc(C#N)cc4)ccc2c2oc(-c4ccc(-c5ccc6ncsc6c5)cc4)nc32)cc1 ; N#Cc1ccc(-c2ccc3c4ccc(-c5ccc(C#N)cc5)cc4c4oc(-c5ccc(-c6ccc7nc(-c8ccccc8)sc7c6)cc5)nc4c3c2)cc1 ; N#Cc1ccc(-c2ccc3c(c2)c2cc(-c4ccc(C#N)cc4)ccc2c2oc(-c4ccc(-c5ccc6nc(-c7ccccc7)sc6c5)cc4)nc32)cc1]

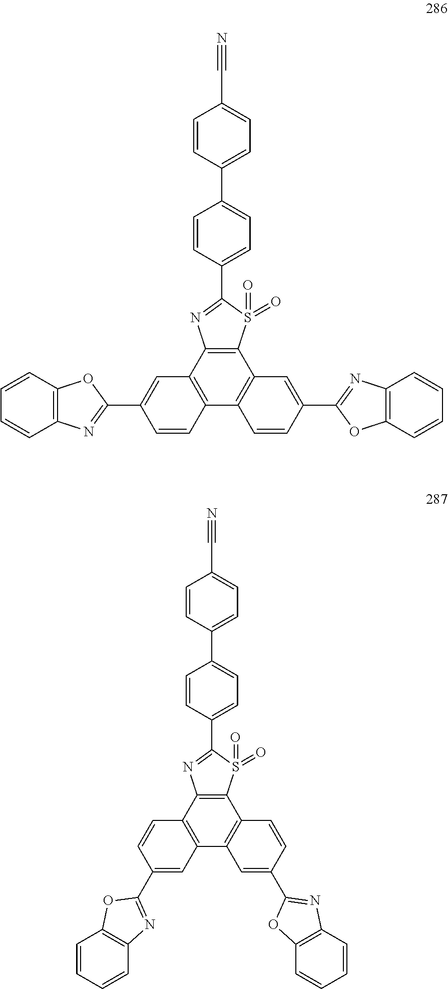 [SMILES: N#Cc1ccc(-c2ccc(C3=Nc4c(c5cc(-c6nc7ccccc7o6)ccc5c5ccc(-c6nc7ccccc7o6)cc45)S3(=O)=O)cc2)cc1 ; N#Cc1ccc(-c2ccc(C3=Nc4c(c5ccc(-c6nc7ccccc7o6)cc5c5cc(-c6nc7ccccc7o6)ccc45)S3(=O)=O)cc2)cc1]

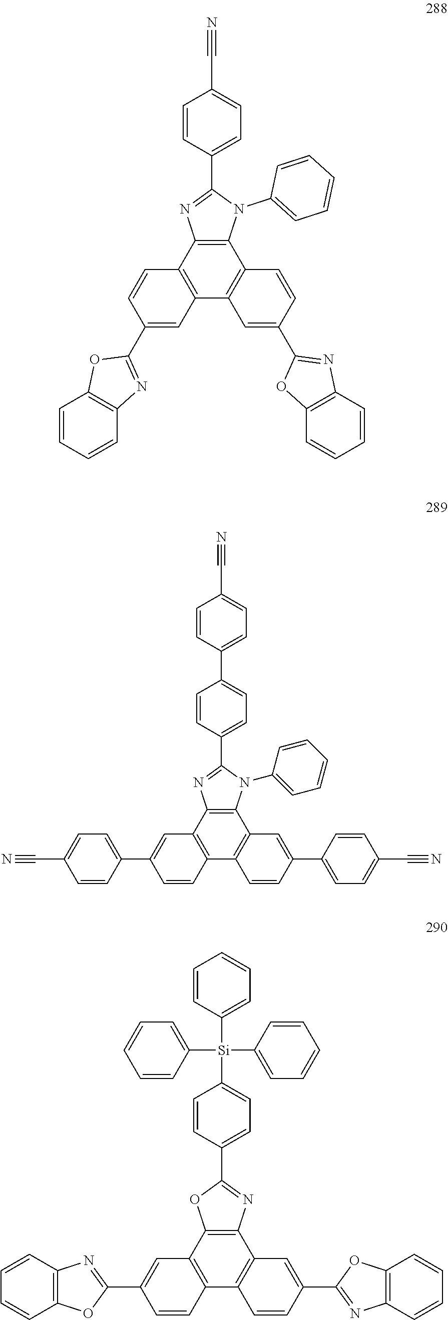 [SMILES: N#Cc1ccc(-c2nc3c4ccc(-c5nc6ccccc6o5)cc4c4cc(-c5nc6ccccc6o5)ccc4c3n2-c2ccccc2)cc1 ; N#Cc1ccc(-c2ccc(-c3nc4c5cc(-c6ccc(C#N)cc6)ccc5c5ccc(-c6ccc(C#N)cc6)cc5c4n3-c3ccccc3)cc2)cc1 ; c1ccc([Si](c2ccccc2)(c2ccccc2)c2ccc(-c3nc4c5cc(-c6nc7ccccc7o6)ccc5c5ccc(-c6nc7ccccc7o6)cc5c4o3)cc2)cc1]

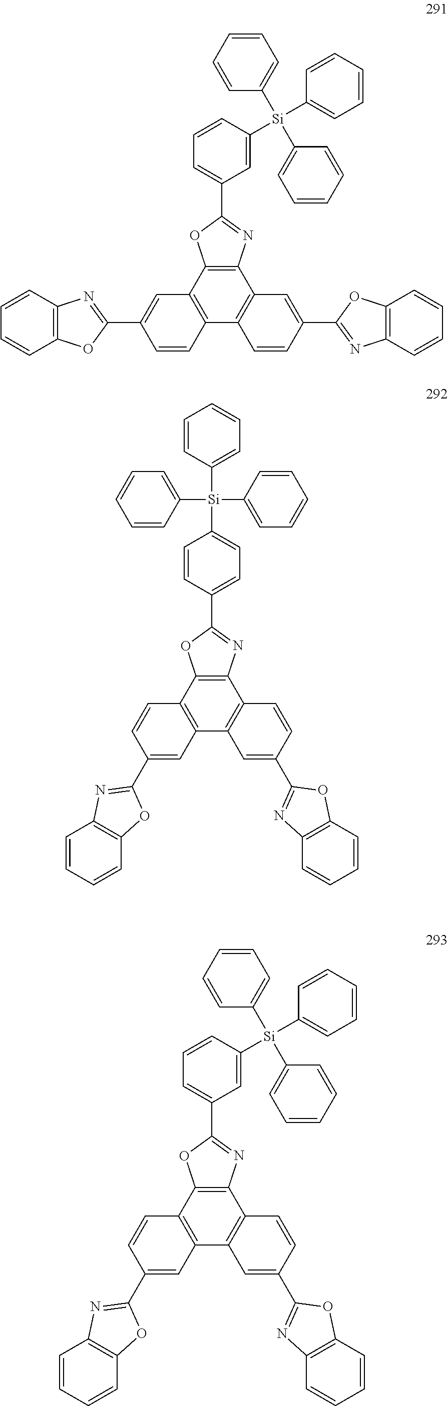 [SMILES: c1ccc([Si](c2ccccc2)(c2ccccc2)c2cccc(-c3nc4c5cc(-c6nc7ccccc7o6)ccc5c5ccc(-c6nc7ccccc7o6)cc5c4o3)c2)cc1 ; c1ccc([Si](c2ccccc2)(c2ccccc2)c2ccc(-c3nc4c5ccc(-c6nc7ccccc7o6)cc5c5cc(-c6nc7ccccc7o6)ccc5c4o3)cc2)cc1 ; c1ccc([Si](c2ccccc2)(c2ccccc2)c2cccc(-c3nc4c5ccc(-c6nc7ccccc7o6)cc5c5cc(-c6nc7ccccc7o6)ccc5c4o3)c2)cc1]

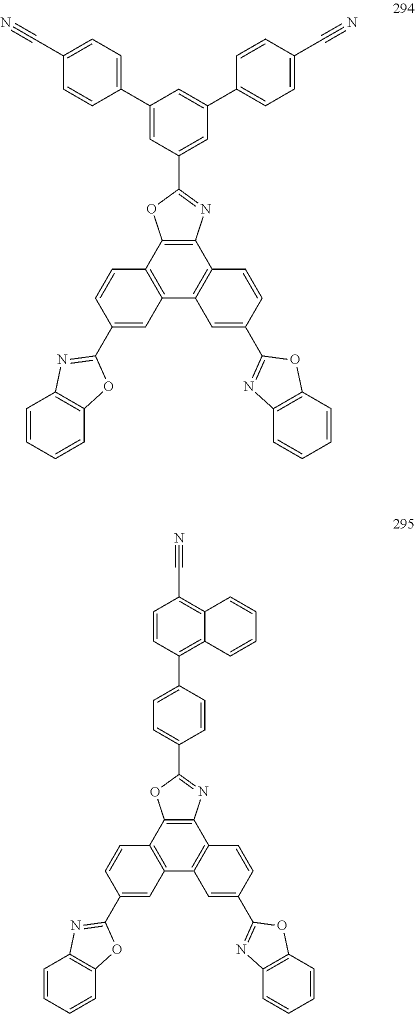 [SMILES: N#Cc1ccc(-c2cc(-c3ccc(C#N)cc3)cc(-c3nc4c5ccc(-c6nc7ccccc7o6)cc5c5cc(-c6nc7ccccc7o6)ccc5c4o3)c2)cc1 ; N#Cc1ccc(-c2ccc(-c3nc4c5ccc(-c6nc7ccccc7o6)cc5c5cc(-c6nc7ccccc7o6)ccc5c4o3)cc2)c2ccccc12]

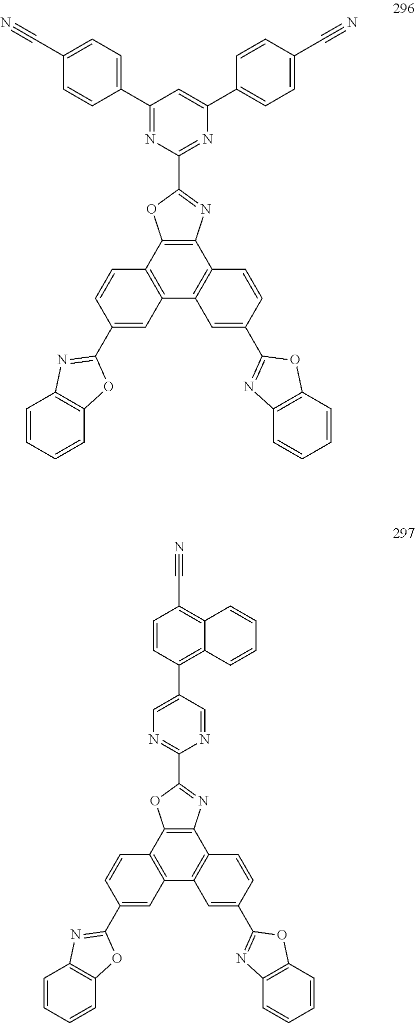 [SMILES: N#Cc1ccc(-c2cc(-c3ccc(C#N)cc3)nc(-c3nc4c5ccc(-c6nc7ccccc7o6)cc5c5cc(-c6nc7ccccc7o6)ccc5c4o3)n2)cc1 ; N#Cc1ccc(-c2cnc(-c3nc4c5ccc(-c6nc7ccccc7o6)cc5c5cc(-c6nc7ccccc7o6)ccc5c4o3)nc2)c2ccccc12]

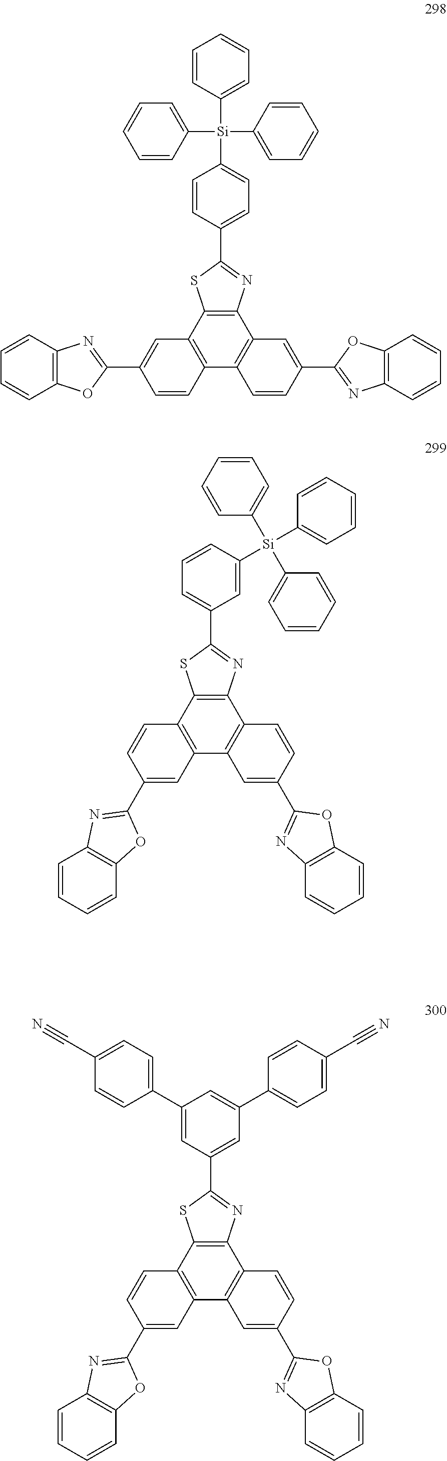 [SMILES: c1ccc([Si](c2ccccc2)(c2ccccc2)c2ccc(-c3nc4c5cc(-c6nc7ccccc7o6)ccc5c5ccc(-c6nc7ccccc7o6)cc5c4s3)cc2)cc1 ; c1ccc([Si](c2ccccc2)(c2ccccc2)c2cccc(-c3nc4c5ccc(-c6nc7ccccc7o6)cc5c5cc(-c6nc7ccccc7o6)ccc5c4s3)c2)cc1 ; N#Cc1ccc(-c2cc(-c3ccc(C#N)cc3)cc(-c3nc4c5ccc(-c6nc7ccccc7o6)cc5c5cc(-c6nc7ccccc7o6)ccc5c4s3)c2)cc1]

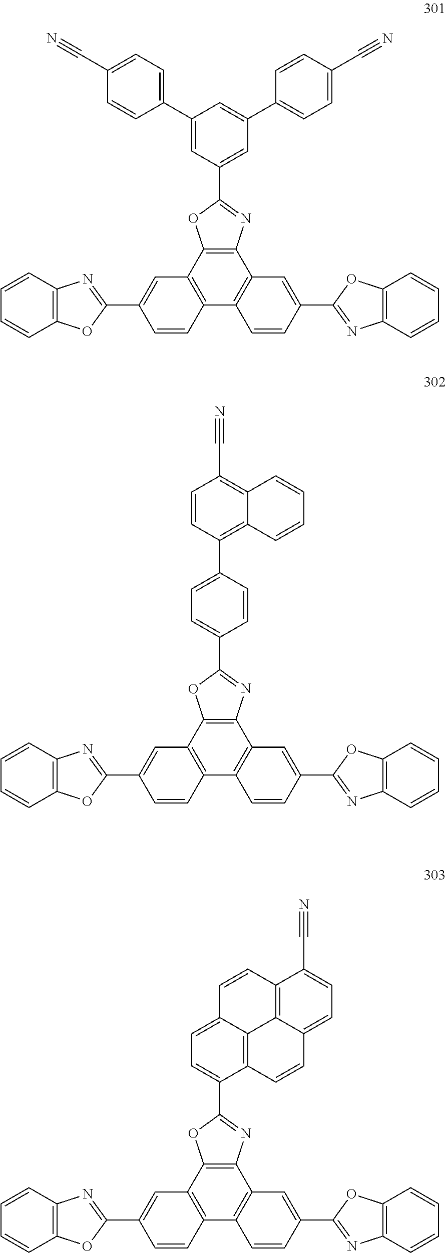 [SMILES: N#Cc1ccc(-c2cc(-c3ccc(C#N)cc3)cc(-c3nc4c5cc(-c6nc7ccccc7o6)ccc5c5ccc(-c6nc7ccccc7o6)cc5c4o3)c2)cc1 ; N#Cc1ccc(-c2ccc(-c3nc4c5cc(-c6nc7ccccc7o6)ccc5c5ccc(-c6nc7ccccc7o6)cc5c4o3)cc2)c2ccccc12 ; N#Cc1ccc2ccc3c(-c4nc5c6cc(-c7nc8ccccc8o7)ccc6c6ccc(-c7nc8ccccc8o7)cc6c5o4)ccc4ccc1c2c43]

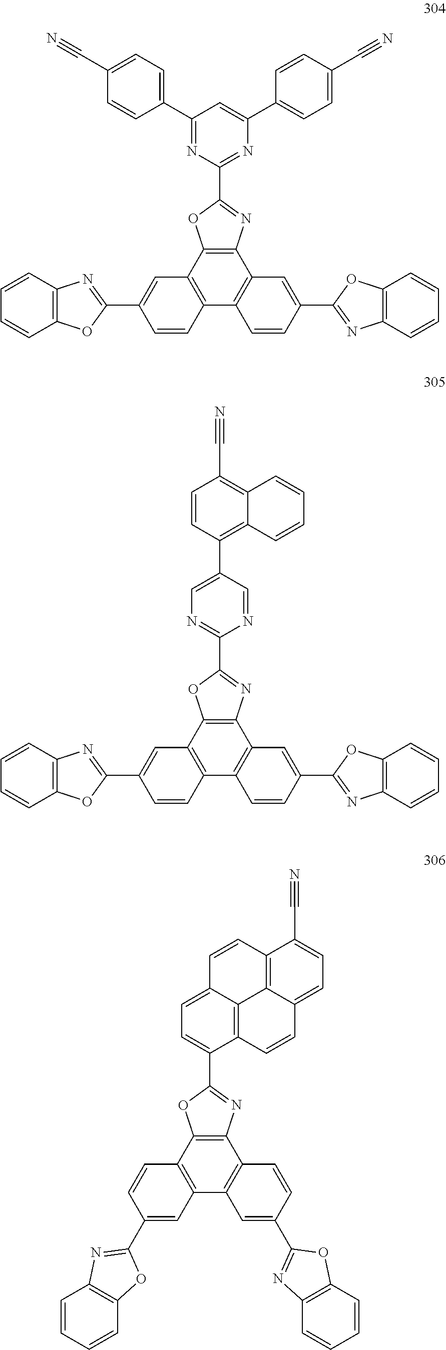 [SMILES: N#Cc1ccc(-c2cc(-c3ccc(C#N)cc3)nc(-c3nc4c5cc(-c6nc7ccccc7o6)ccc5c5ccc(-c6nc7ccccc7o6)cc5c4o3)n2)cc1 ; N#Cc1ccc(-c2cnc(-c3nc4c5cc(-c6nc7ccccc7o6)ccc5c5ccc(-c6nc7ccccc7o6)cc5c4o3)nc2)c2ccccc12 ; N#Cc1ccc2ccc3c(-c4nc5c6ccc(-c7nc8ccccc8o7)cc6c6cc(-c7nc8ccccc8o7)ccc6c5o4)ccc4ccc1c2c43]

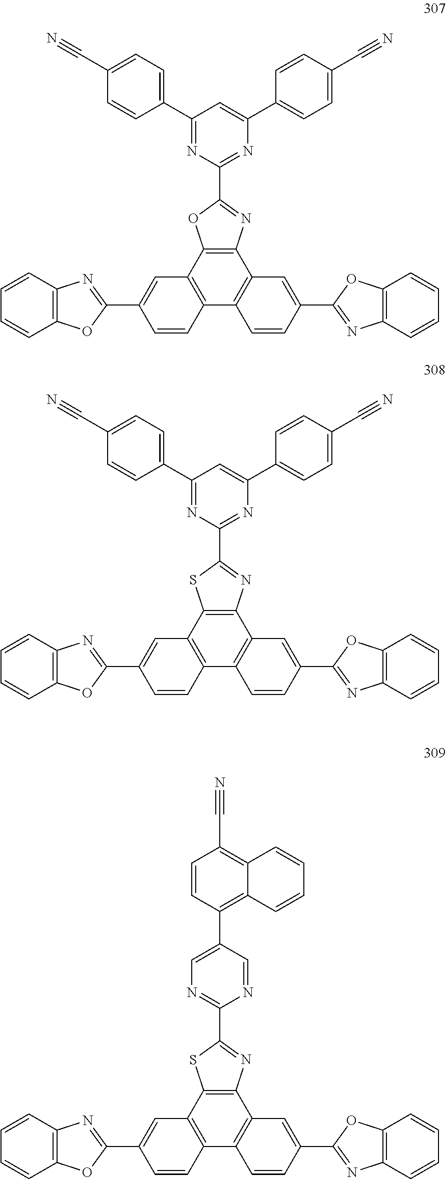 [SMILES: N#Cc1ccc(-c2cc(-c3ccc(C#N)cc3)nc(-c3nc4c5cc(-c6nc7ccccc7o6)ccc5c5ccc(-c6nc7ccccc7o6)cc5c4o3)n2)cc1 ; N#Cc1ccc(-c2cc(-c3ccc(C#N)cc3)nc(-c3nc4c5cc(-c6nc7ccccc7o6)ccc5c5ccc(-c6nc7ccccc7o6)cc5c4s3)n2)cc1 ; N#Cc1ccc(-c2cnc(-c3nc4c5cc(-c6nc7ccccc7o6)ccc5c5ccc(-c6nc7ccccc7o6)cc5c4s3)nc2)c2ccccc12]

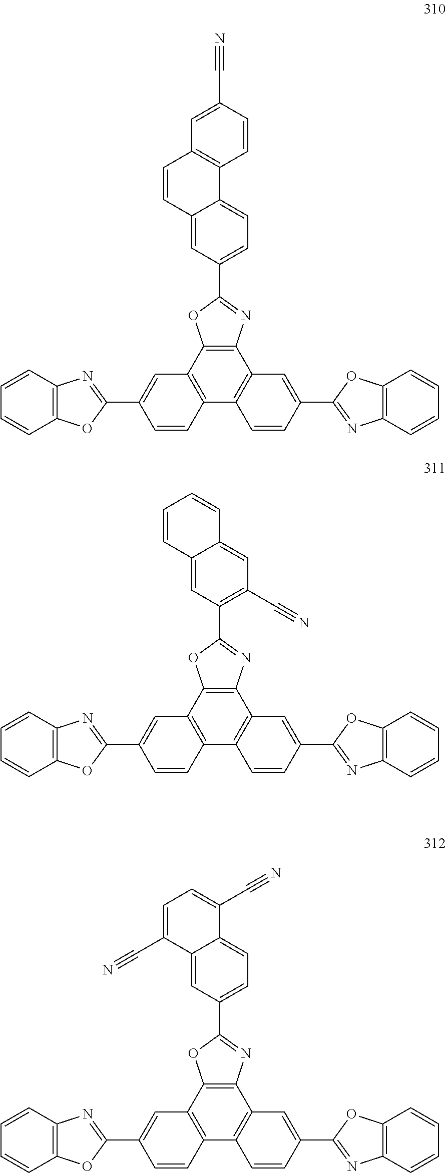 [SMILES: N#Cc1ccc2c(ccc3cc(-c4nc5c6cc(-c7nc8ccccc8o7)ccc6c6ccc(-c7nc8ccccc8o7)cc6c5o4)ccc32)c1 ; N#Cc1cc2ccccc2cc1-c1nc2c3cc(-c4nc5ccccc5o4)ccc3c3ccc(-c4nc5ccccc5o4)cc3c2o1 ; N#Cc1ccc(C#N)c2cc(-c3nc4c5cc(-c6nc7ccccc7o6)ccc5c5ccc(-c6nc7ccccc7o6)cc5c4o3)ccc12]

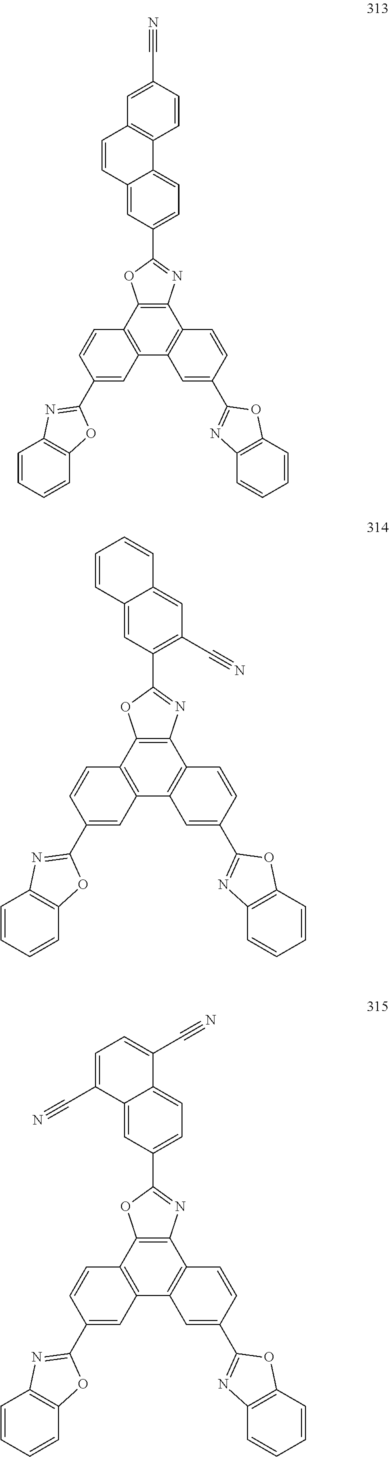 [SMILES: N#Cc1ccc2c(ccc3cc(-c4nc5c6ccc(-c7nc8ccccc8o7)cc6c6cc(-c7nc8ccccc8o7)ccc6c5o4)ccc32)c1 ; N#Cc1cc2ccccc2cc1-c1nc2c3ccc(-c4nc5ccccc5o4)cc3c3cc(-c4nc5ccccc5o4)ccc3c2o1 ; N#Cc1ccc(C#N)c2cc(-c3nc4c5ccc(-c6nc7ccccc7o6)cc5c5cc(-c6nc7ccccc7o6)ccc5c4o3)ccc12]

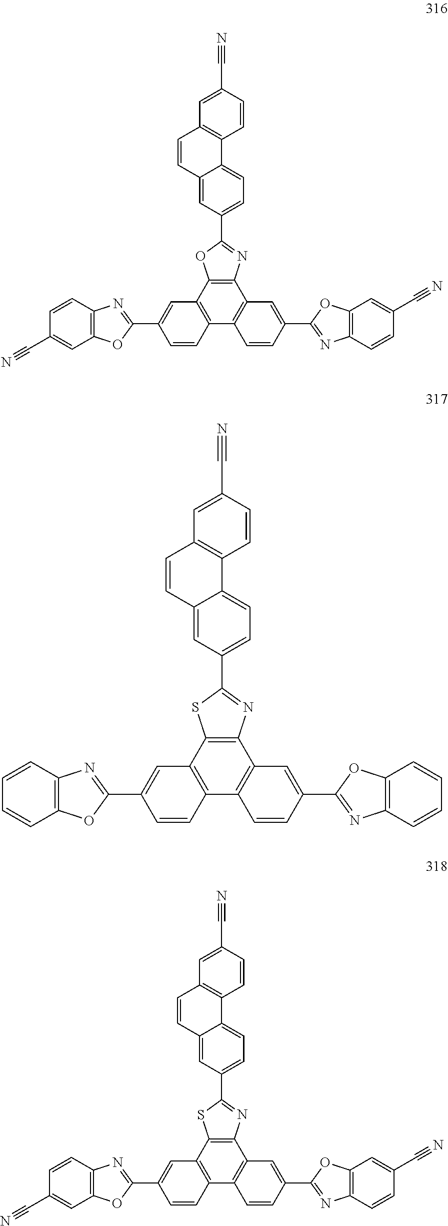 [SMILES: N#Cc1ccc2c(ccc3cc(-c4nc5c6cc(-c7nc8ccc(C#N)cc8o7)ccc6c6ccc(-c7nc8ccc(C#N)cc8o7)cc6c5o4)ccc32)c1 ; N#Cc1ccc2c(ccc3cc(-c4nc5c6cc(-c7nc8ccccc8o7)ccc6c6ccc(-c7nc8ccccc8o7)cc6c5s4)ccc32)c1 ; N#Cc1ccc2c(ccc3cc(-c4nc5c6cc(-c7nc8ccc(C#N)cc8o7)ccc6c6ccc(-c7nc8ccc(C#N)cc8o7)cc6c5s4)ccc32)c1]

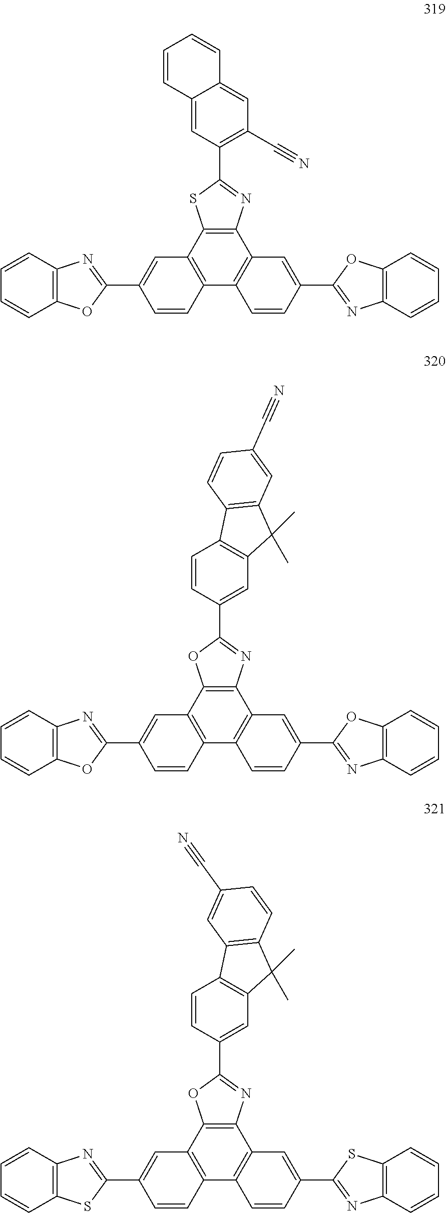 [SMILES: N#Cc1cc2ccccc2cc1-c1nc2c3cc(-c4nc5ccccc5o4)ccc3c3ccc(-c4nc5ccccc5o4)cc3c2s1 ; CC1(C)c2cc(C#N)ccc2-c2ccc(-c3nc4c5cc(-c6nc7ccccc7o6)ccc5c5ccc(-c6nc7ccccc7o6)cc5c4o3)cc21 ; CC1(C)c2ccc(C#N)cc2-c2ccc(-c3nc4c5cc(-c6nc7ccccc7s6)ccc5c5ccc(-c6nc7ccccc7s6)cc5c4o3)cc21]

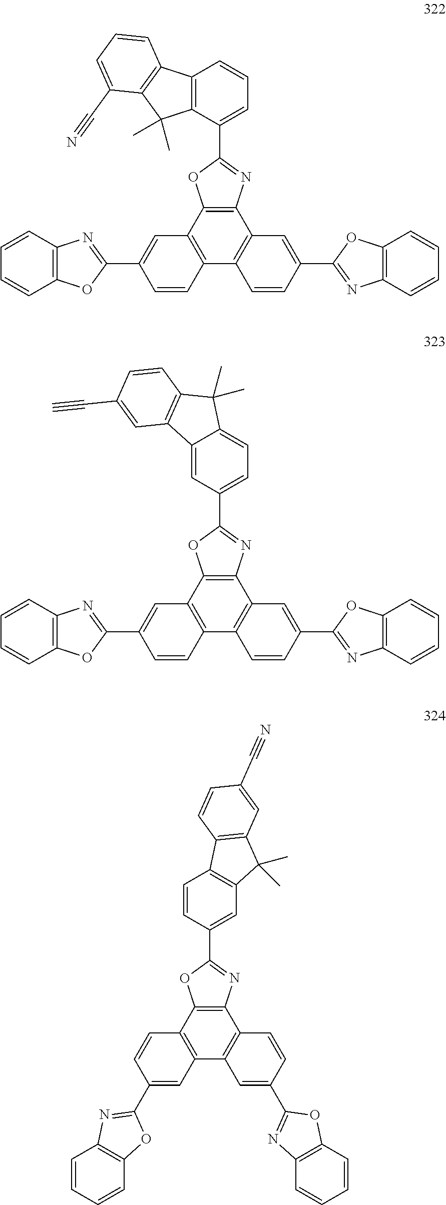 [SMILES: CC1(C)c2c(C#N)cccc2-c2cccc(-c3nc4c5cc(-c6nc7ccccc7o6)ccc5c5ccc(-c6nc7ccccc7o6)cc5c4o3)c21 ; C#Cc1ccc2c(c1)-c1cc(-c3nc4c5cc(-c6nc7ccccc7o6)ccc5c5ccc(-c6nc7ccccc7o6)cc5c4o3)ccc1C2(C)C ; CC1(C)c2cc(C#N)ccc2-c2ccc(-c3nc4c5ccc(-c6nc7ccccc7o6)cc5c5cc(-c6nc7ccccc7o6)ccc5c4o3)cc21]

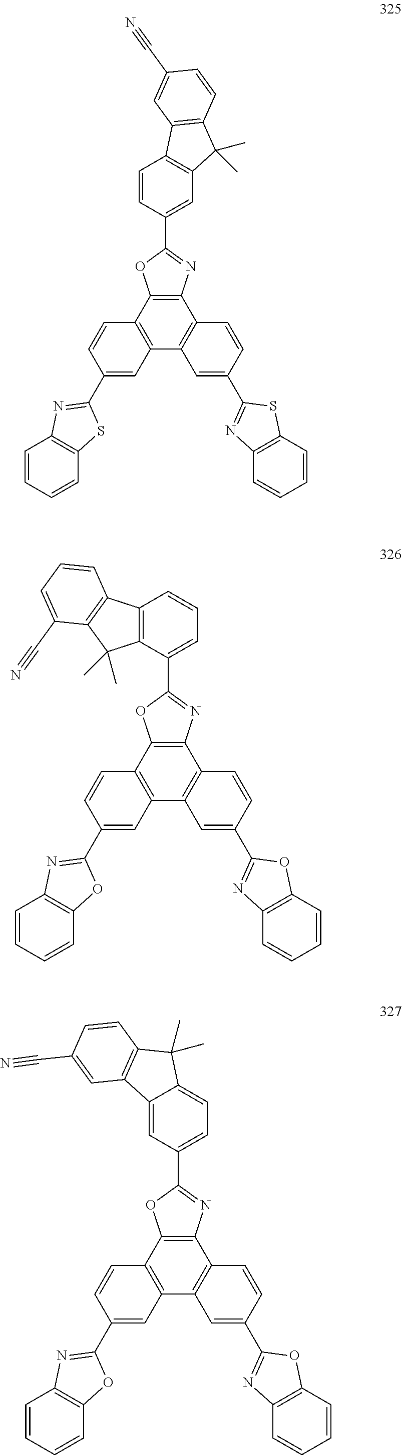 [SMILES: CC1(C)c2ccc(C#N)cc2-c2ccc(-c3nc4c5ccc(-c6nc7ccccc7s6)cc5c5cc(-c6nc7ccccc7s6)ccc5c4o3)cc21 ; CC1(C)c2c(C#N)cccc2-c2cccc(-c3nc4c5ccc(-c6nc7ccccc7o6)cc5c5cc(-c6nc7ccccc7o6)ccc5c4o3)c21 ; CC1(C)c2ccc(C#N)cc2-c2cc(-c3nc4c5ccc(-c6nc7ccccc7o6)cc5c5cc(-c6nc7ccccc7o6)ccc5c4o3)ccc21]

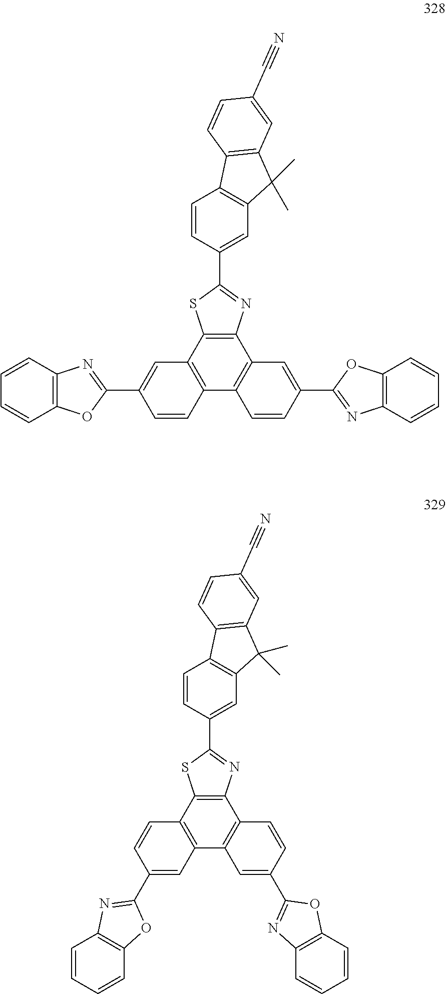 [SMILES: CC1(C)c2cc(C#N)ccc2-c2ccc(-c3nc4c5cc(-c6nc7ccccc7o6)ccc5c5ccc(-c6nc7ccccc7o6)cc5c4s3)cc21 ; CC1(C)c2cc(C#N)ccc2-c2ccc(-c3nc4c5ccc(-c6nc7ccccc7o6)cc5c5cc(-c6nc7ccccc7o6)ccc5c4s3)cc21]

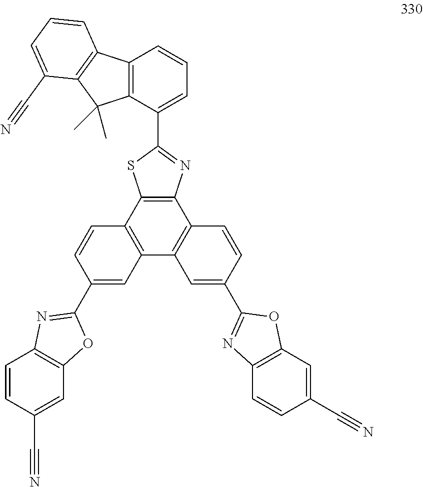 [SMILES: CC1(C)c2c(C#N)cccc2-c2cccc(-c3nc4c5ccc(-c6nc7ccc(C#N)cc7o6)cc5c5cc(-c6nc7ccc(C#N)cc7o6)ccc5c4s3)c21]

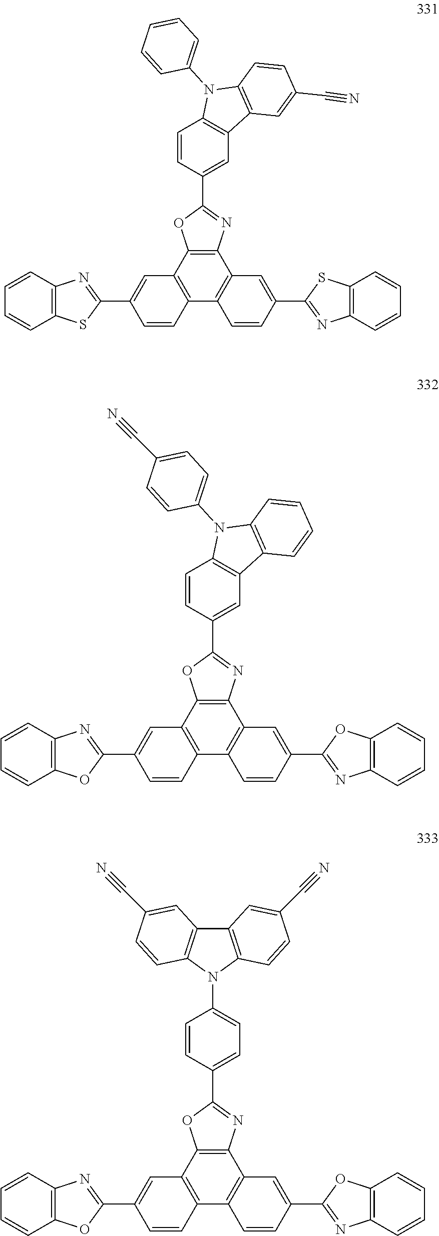 [SMILES: N#Cc1ccc2c(c1)c1cc(-c3nc4c5cc(-c6nc7ccccc7s6)ccc5c5ccc(-c6nc7ccccc7s6)cc5c4o3)ccc1n2-c1ccccc1 ; N#Cc1ccc(-n2c3ccccc3c3cc(-c4nc5c6cc(-c7nc8ccccc8o7)ccc6c6ccc(-c7nc8ccccc8o7)cc6c5o4)ccc32)cc1 ; N#Cc1ccc2c(c1)c1cc(C#N)ccc1n2-c1ccc(-c2nc3c4cc(-c5nc6ccccc6o5)ccc4c4ccc(-c5nc6ccccc6o5)cc4c3o2)cc1]

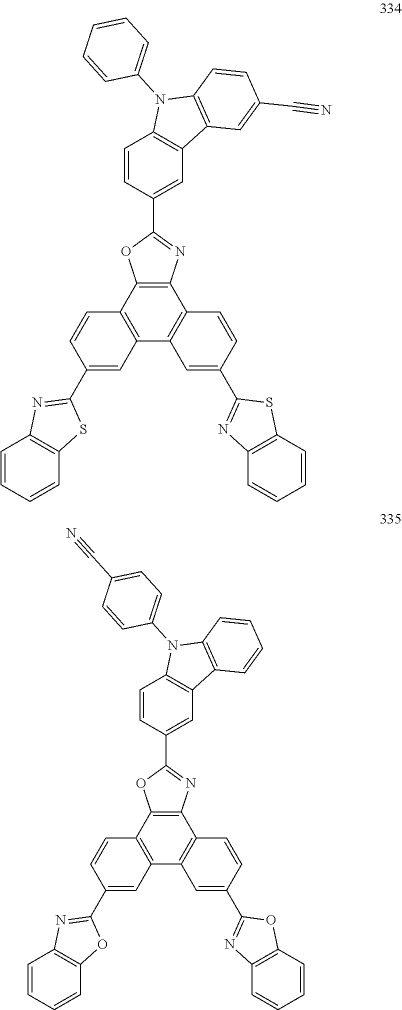 [SMILES: N#Cc1ccc2c(c1)c1cc(-c3nc4c5ccc(-c6nc7ccccc7s6)cc5c5cc(-c6nc7ccccc7s6)ccc5c4o3)ccc1n2-c1ccccc1 ; N#Cc1ccc(-n2c3ccccc3c3cc(-c4nc5c6ccc(-c7nc8ccccc8o7)cc6c6cc(-c7nc8ccccc8o7)ccc6c5o4)ccc32)cc1]

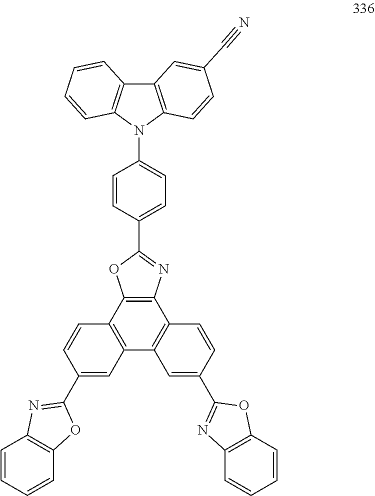 [SMILES: N#Cc1ccc2c(c1)c1ccccc1n2-c1ccc(-c2nc3c4ccc(-c5nc6ccccc6o5)cc4c4cc(-c5nc6ccccc6o5)ccc4c3o2)cc1]

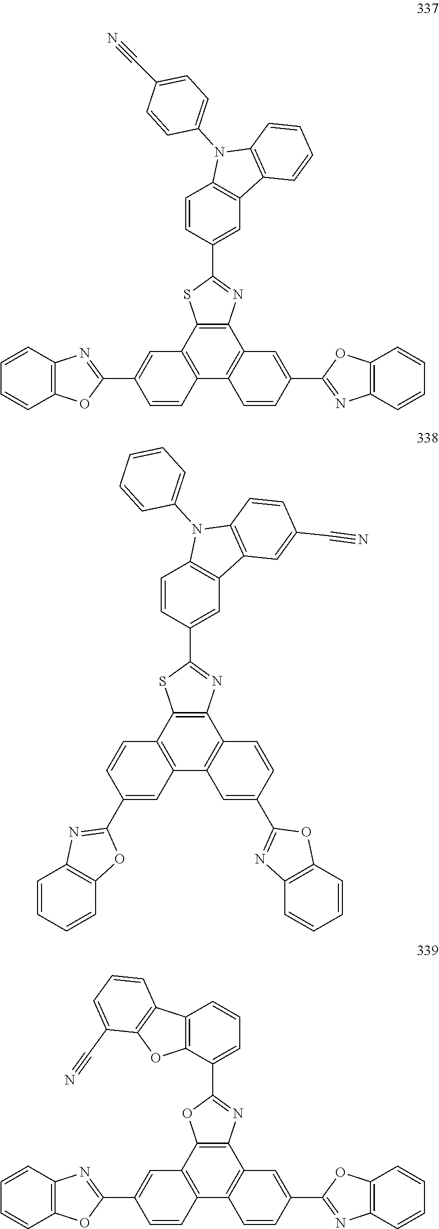 [SMILES: N#Cc1ccc(-n2c3ccccc3c3cc(-c4nc5c6cc(-c7nc8ccccc8o7)ccc6c6ccc(-c7nc8ccccc8o7)cc6c5s4)ccc32)cc1 ; N#Cc1ccc2c(c1)c1cc(-c3nc4c5ccc(-c6nc7ccccc7o6)cc5c5cc(-c6nc7ccccc7o6)ccc5c4s3)ccc1n2-c1ccccc1 ; N#Cc1cccc2c1oc1c(-c3nc4c5cc(-c6nc7ccccc7o6)ccc5c5ccc(-c6nc7ccccc7o6)cc5c4o3)cccc12]

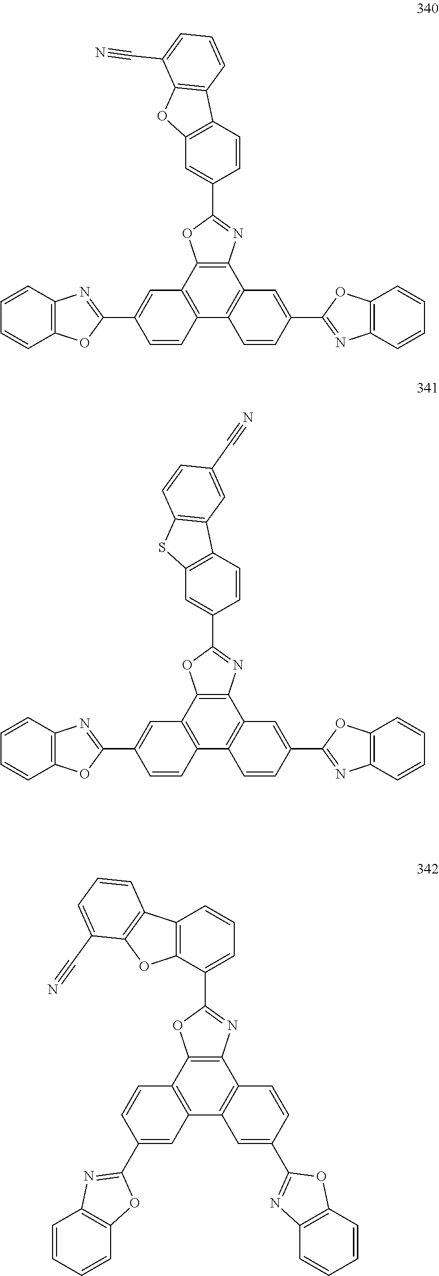 [SMILES: N#Cc1cccc2c1oc1cc(-c3nc4c5cc(-c6nc7ccccc7o6)ccc5c5ccc(-c6nc7ccccc7o6)cc5c4o3)ccc12 ; N#Cc1ccc2sc3cc(-c4nc5c6cc(-c7nc8ccccc8o7)ccc6c6ccc(-c7nc8ccccc8o7)cc6c5o4)ccc3c2c1 ; N#Cc1cccc2c1oc1c(-c3nc4c5ccc(-c6nc7ccccc7o6)cc5c5cc(-c6nc7ccccc7o6)ccc5c4o3)cccc12]

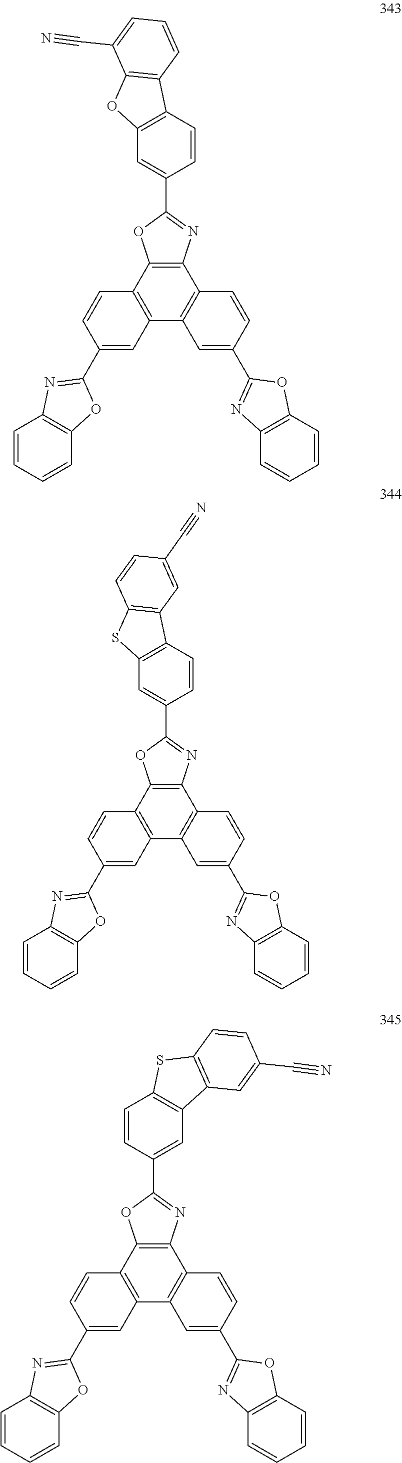 [SMILES: N#Cc1cccc2c1oc1cc(-c3nc4c5ccc(-c6nc7ccccc7o6)cc5c5cc(-c6nc7ccccc7o6)ccc5c4o3)ccc12 ; N#Cc1ccc2sc3cc(-c4nc5c6ccc(-c7nc8ccccc8o7)cc6c6cc(-c7nc8ccccc8o7)ccc6c5o4)ccc3c2c1 ; N#Cc1ccc2sc3ccc(-c4nc5c6ccc(-c7nc8ccccc8o7)cc6c6cc(-c7nc8ccccc8o7)ccc6c5o4)cc3c2c1]

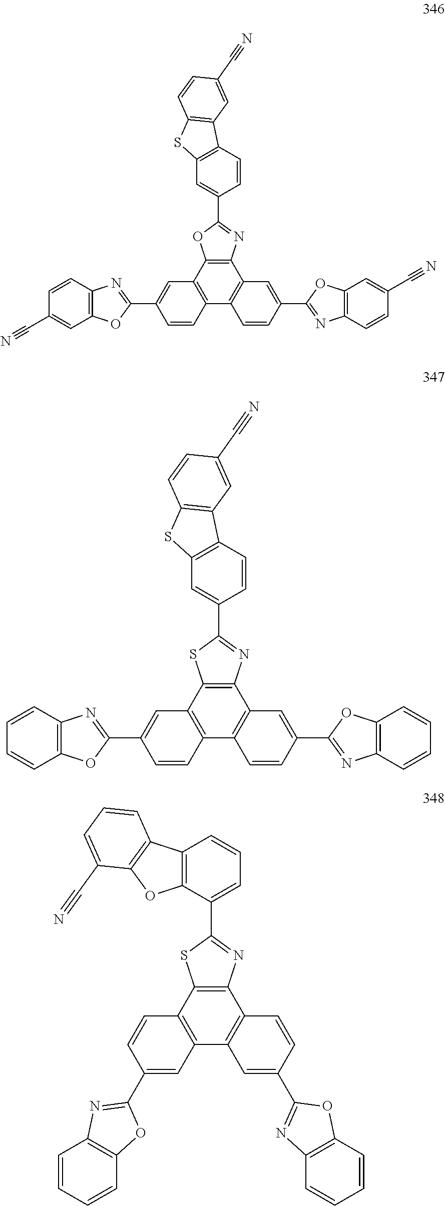 [SMILES: N#Cc1ccc2nc(-c3ccc4c5ccc(-c6nc7ccc(C#N)cc7o6)cc5c5oc(-c6ccc7c(c6)sc6ccc(C#N)cc67)nc5c4c3)oc2c1 ; N#Cc1ccc2sc3cc(-c4nc5c6cc(-c7nc8ccccc8o7)ccc6c6ccc(-c7nc8ccccc8o7)cc6c5s4)ccc3c2c1 ; N#Cc1cccc2c1oc1c(-c3nc4c5ccc(-c6nc7ccccc7o6)cc5c5cc(-c6nc7ccccc7o6)ccc5c4s3)cccc12]

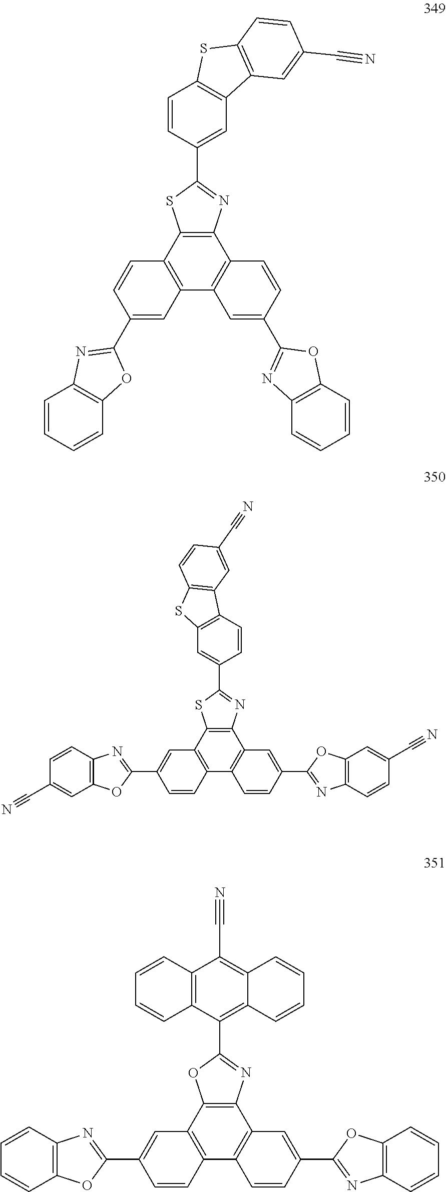 [SMILES: N#Cc1ccc2sc3ccc(-c4nc5c6ccc(-c7nc8ccccc8o7)cc6c6cc(-c7nc8ccccc8o7)ccc6c5s4)cc3c2c1 ; N#Cc1ccc2nc(-c3ccc4c5ccc(-c6nc7ccc(C#N)cc7o6)cc5c5sc(-c6ccc7c(c6)sc6ccc(C#N)cc67)nc5c4c3)oc2c1 ; N#Cc1c2ccccc2c(-c2nc3c4cc(-c5nc6ccccc6o5)ccc4c4ccc(-c5nc6ccccc6o5)cc4c3o2)c2ccccc12]

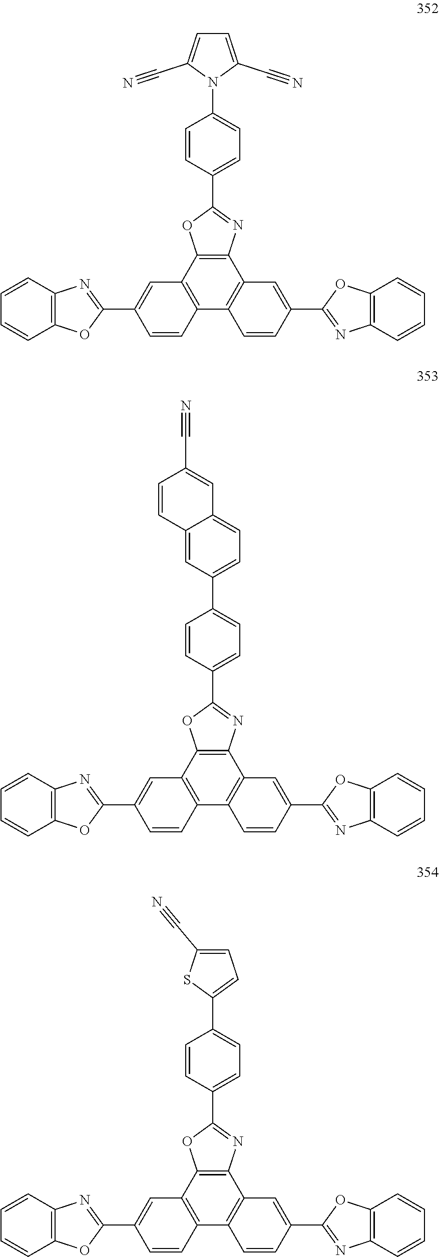 [SMILES: N#Cc1ccc(C#N)n1-c1ccc(-c2nc3c4cc(-c5nc6ccccc6o5)ccc4c4ccc(-c5nc6ccccc6o5)cc4c3o2)cc1 ; N#Cc1ccc2cc(-c3ccc(-c4nc5c6cc(-c7nc8ccccc8o7)ccc6c6ccc(-c7nc8ccccc8o7)cc6c5o4)cc3)ccc2c1 ; N#Cc1ccc(-c2ccc(-c3nc4c5cc(-c6nc7ccccc7o6)ccc5c5ccc(-c6nc7ccccc7o6)cc5c4o3)cc2)s1]

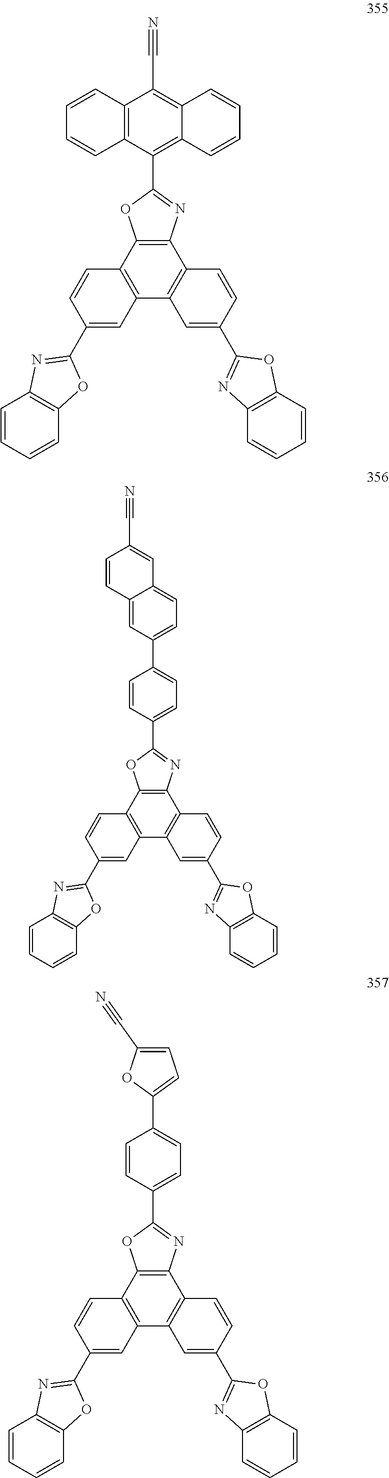 [SMILES: N#Cc1c2ccccc2c(-c2nc3c4ccc(-c5nc6ccccc6o5)cc4c4cc(-c5nc6ccccc6o5)ccc4c3o2)c2ccccc12 ; N#Cc1ccc2cc(-c3ccc(-c4nc5c6ccc(-c7nc8ccccc8o7)cc6c6cc(-c7nc8ccccc8o7)ccc6c5o4)cc3)ccc2c1 ; N#Cc1ccc(-c2ccc(-c3nc4c5ccc(-c6nc7ccccc7o6)cc5c5cc(-c6nc7ccccc7o6)ccc5c4o3)cc2)o1]

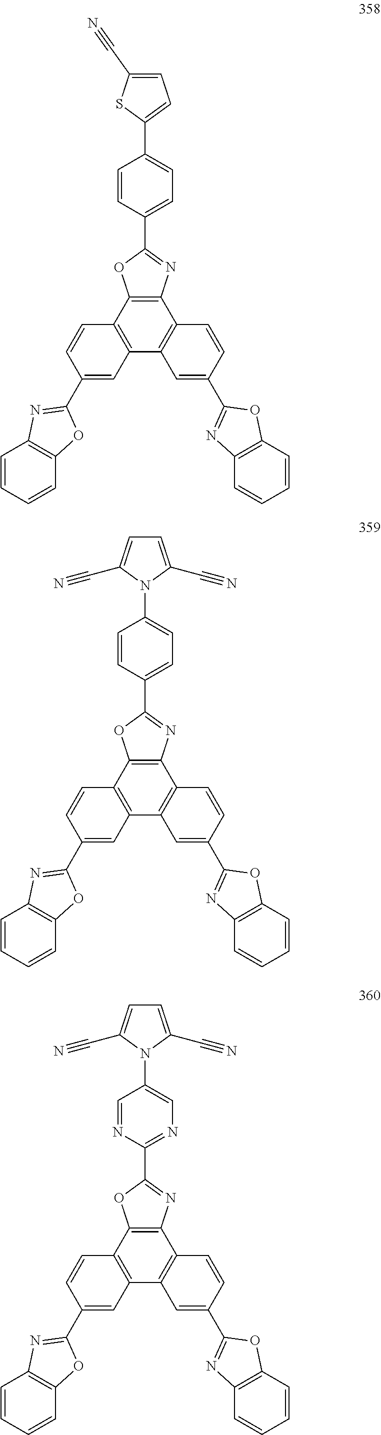 [SMILES: N#Cc1ccc(-c2ccc(-c3nc4c5ccc(-c6nc7ccccc7o6)cc5c5cc(-c6nc7ccccc7o6)ccc5c4o3)cc2)s1 ; N#Cc1ccc(C#N)n1-c1ccc(-c2nc3c4ccc(-c5nc6ccccc6o5)cc4c4cc(-c5nc6ccccc6o5)ccc4c3o2)cc1 ; N#Cc1ccc(C#N)n1-c1cnc(-c2nc3c4ccc(-c5nc6ccccc6o5)cc4c4cc(-c5nc6ccccc6o5)ccc4c3o2)nc1]

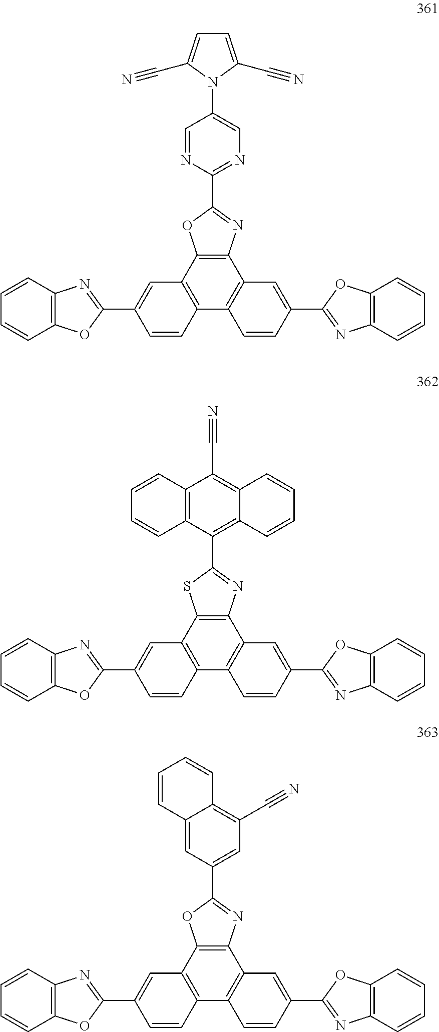 [SMILES: N#Cc1ccc(C#N)n1-c1cnc(-c2nc3c4cc(-c5nc6ccccc6o5)ccc4c4ccc(-c5nc6ccccc6o5)cc4c3o2)nc1 ; N#Cc1c2ccccc2c(-c2nc3c4cc(-c5nc6ccccc6o5)ccc4c4ccc(-c5nc6ccccc6o5)cc4c3s2)c2ccccc12 ; N#Cc1cc(-c2nc3c4cc(-c5nc6ccccc6o5)ccc4c4ccc(-c5nc6ccccc6o5)cc4c3o2)cc2ccccc12]

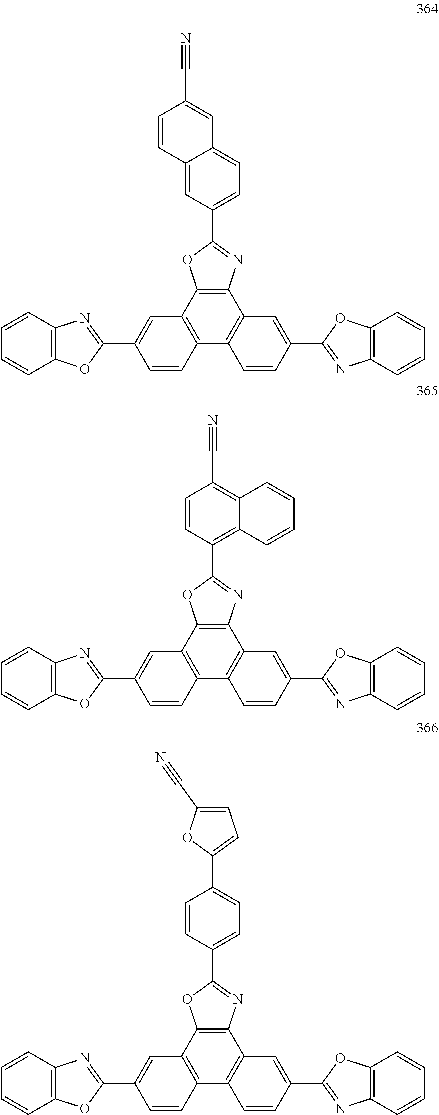 [SMILES: N#Cc1ccc2cc(-c3nc4c5cc(-c6nc7ccccc7o6)ccc5c5ccc(-c6nc7ccccc7o6)cc5c4o3)ccc2c1 ; N#Cc1ccc(-c2nc3c4cc(-c5nc6ccccc6o5)ccc4c4ccc(-c5nc6ccccc6o5)cc4c3o2)c2ccccc12 ; N#Cc1ccc(-c2ccc(-c3nc4c5cc(-c6nc7ccccc7o6)ccc5c5ccc(-c6nc7ccccc7o6)cc5c4o3)cc2)o1]

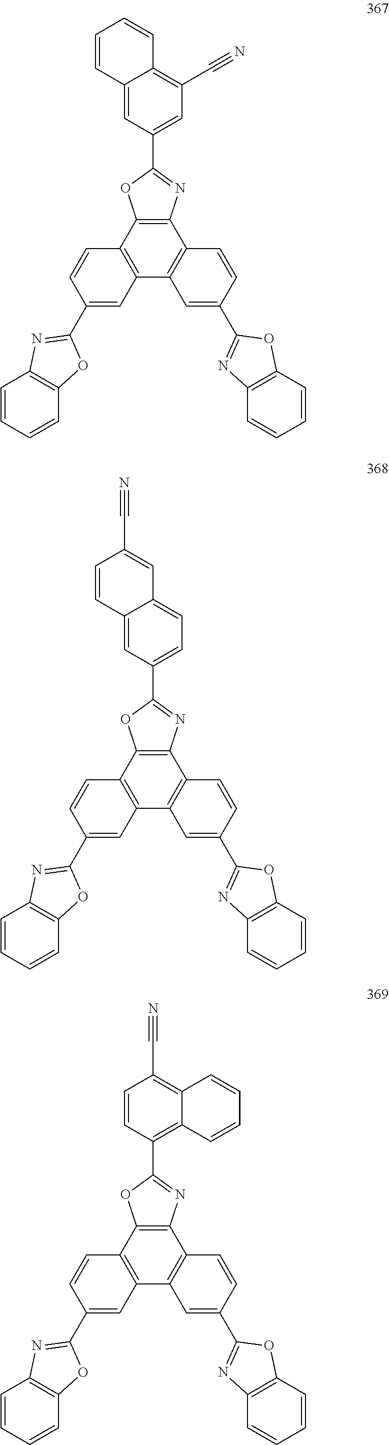 [SMILES: N#Cc1cc(-c2nc3c4ccc(-c5nc6ccccc6o5)cc4c4cc(-c5nc6ccccc6o5)ccc4c3o2)cc2ccccc12 ; N#Cc1ccc2cc(-c3nc4c5ccc(-c6nc7ccccc7o6)cc5c5cc(-c6nc7ccccc7o6)ccc5c4o3)ccc2c1 ; N#Cc1ccc(-c2nc3c4ccc(-c5nc6ccccc6o5)cc4c4cc(-c5nc6ccccc6o5)ccc4c3o2)c2ccccc12]

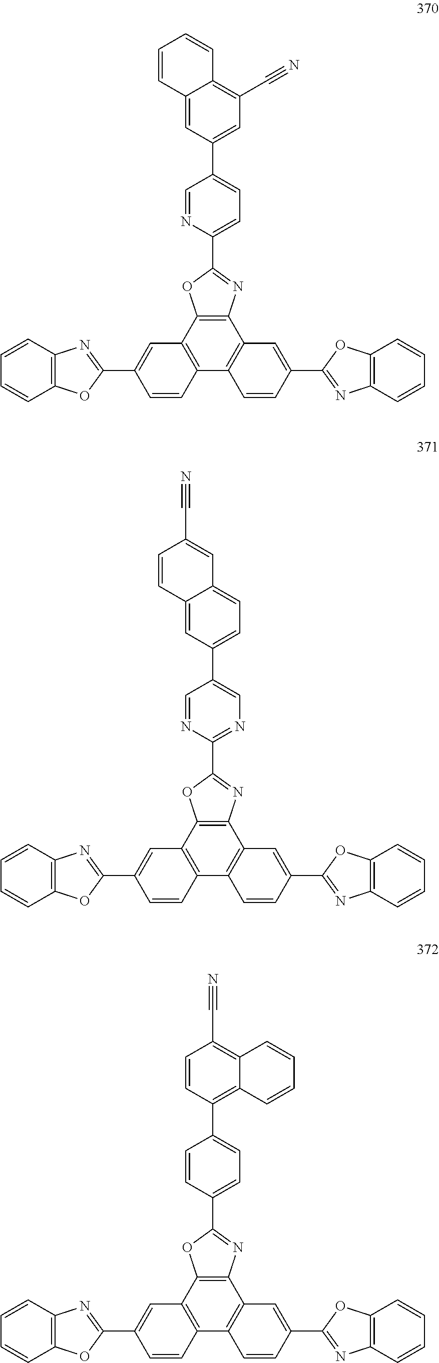 [SMILES: N#Cc1cc(-c2ccc(-c3nc4c5cc(-c6nc7ccccc7o6)ccc5c5ccc(-c6nc7ccccc7o6)cc5c4o3)nc2)cc2ccccc12 ; N#Cc1ccc2cc(-c3cnc(-c4nc5c6cc(-c7nc8ccccc8o7)ccc6c6ccc(-c7nc8ccccc8o7)cc6c5o4)nc3)ccc2c1 ; N#Cc1ccc(-c2ccc(-c3nc4c5cc(-c6nc7ccccc7o6)ccc5c5ccc(-c6nc7ccccc7o6)cc5c4o3)cc2)c2ccccc12]

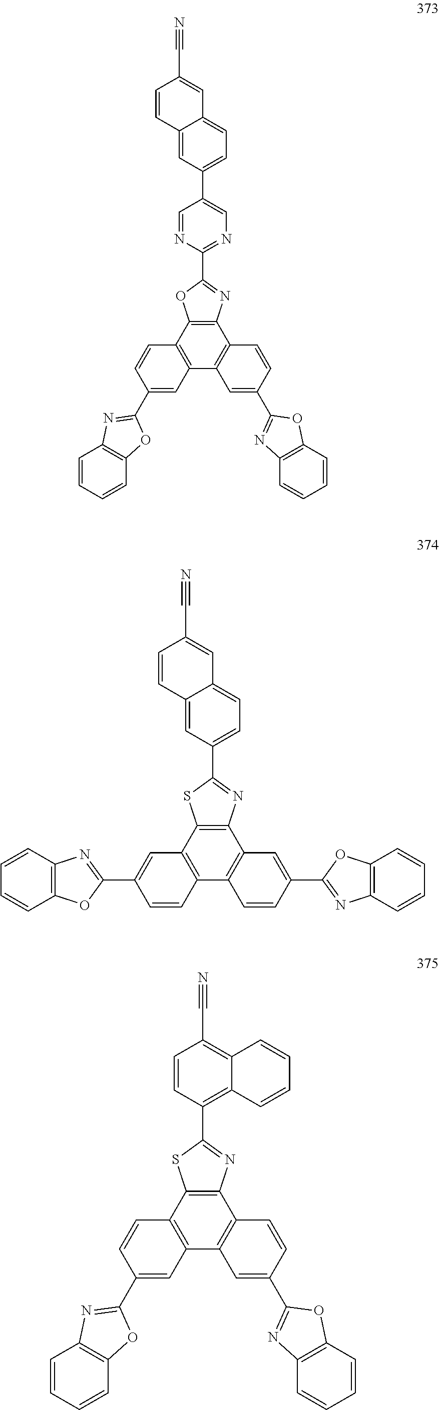 [SMILES: N#Cc1ccc2cc(-c3cnc(-c4nc5c6ccc(-c7nc8ccccc8o7)cc6c6cc(-c7nc8ccccc8o7)ccc6c5o4)nc3)ccc2c1 ; N#Cc1ccc2cc(-c3nc4c5cc(-c6nc7ccccc7o6)ccc5c5ccc(-c6nc7ccccc7o6)cc5c4s3)ccc2c1 ; N#Cc1ccc(-c2nc3c4ccc(-c5nc6ccccc6o5)cc4c4cc(-c5nc6ccccc6o5)ccc4c3s2)c2ccccc12]

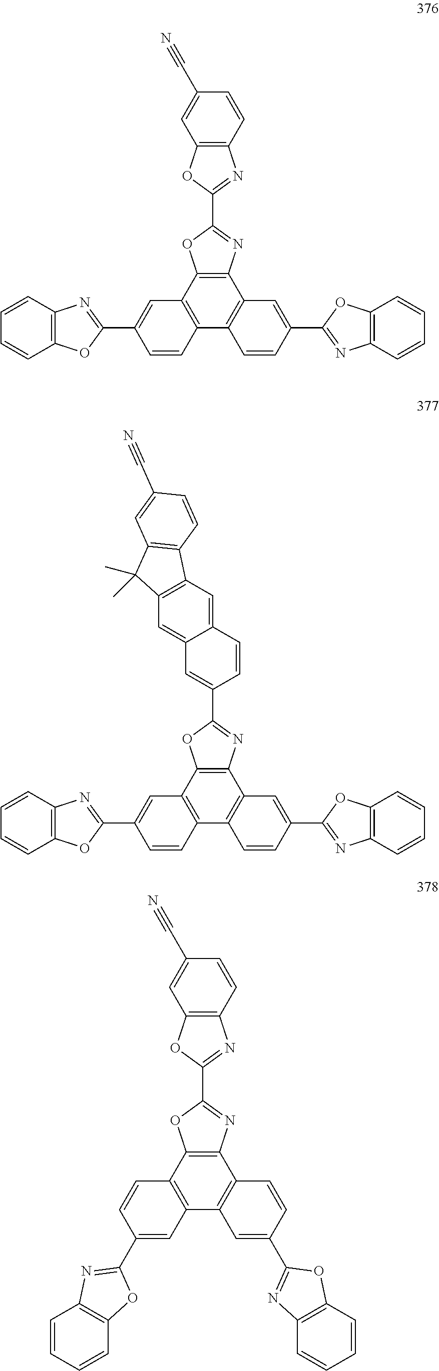 [SMILES: N#Cc1ccc2nc(-c3nc4c5cc(-c6nc7ccccc7o6)ccc5c5ccc(-c6nc7ccccc7o6)cc5c4o3)oc2c1 ; CC1(C)c2cc(C#N)ccc2-c2cc3ccc(-c4nc5c6cc(-c7nc8ccccc8o7)ccc6c6ccc(-c7nc8ccccc8o7)cc6c5o4)cc3cc21 ; N#Cc1ccc2nc(-c3nc4c5ccc(-c6nc7ccccc7o6)cc5c5cc(-c6nc7ccccc7o6)ccc5c4o3)oc2c1]

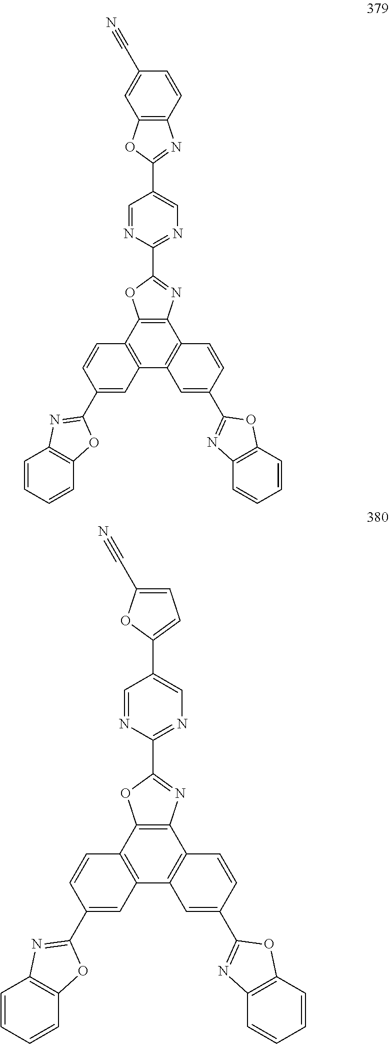 [SMILES: N#Cc1ccc2nc(-c3cnc(-c4nc5c6ccc(-c7nc8ccccc8o7)cc6c6cc(-c7nc8ccccc8o7)ccc6c5o4)nc3)oc2c1 ; N#Cc1ccc(-c2cnc(-c3nc4c5ccc(-c6nc7ccccc7o6)cc5c5cc(-c6nc7ccccc7o6)ccc5c4o3)nc2)o1]

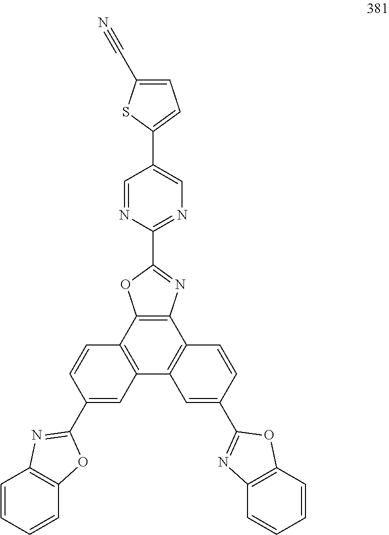 [SMILES: N#Cc1ccc(-c2cnc(-c3nc4c5ccc(-c6nc7ccccc7o6)cc5c5cc(-c6nc7ccccc7o6)ccc5c4o3)nc2)s1]

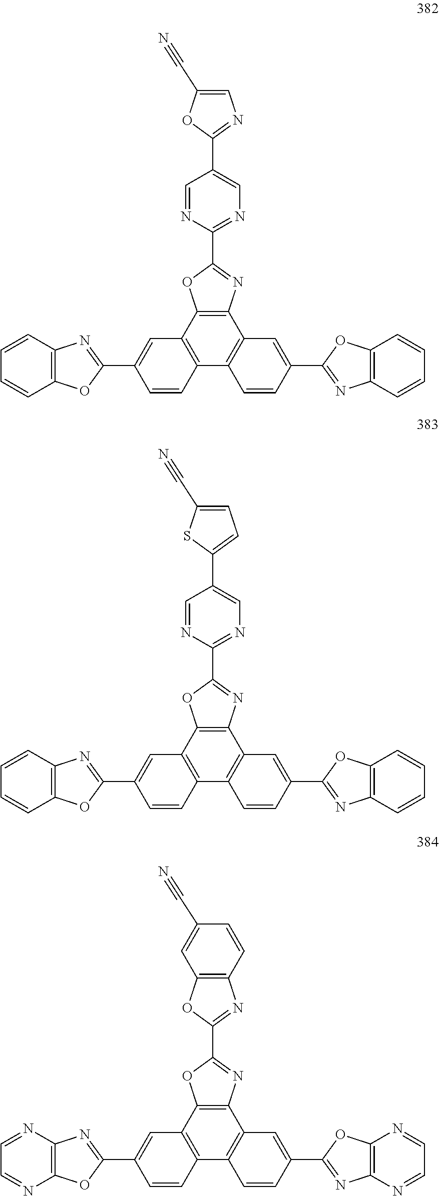 [SMILES: N#Cc1cnc(-c2cnc(-c3nc4c5cc(-c6nc7ccccc7o6)ccc5c5ccc(-c6nc7ccccc7o6)cc5c4o3)nc2)o1 ; N#Cc1ccc(-c2cnc(-c3nc4c5cc(-c6nc7ccccc7o6)ccc5c5ccc(-c6nc7ccccc7o6)cc5c4o3)nc2)s1 ; N#Cc1ccc2nc(-c3nc4c5cc(-c6nc7nccnc7o6)ccc5c5ccc(-c6nc7nccnc7o6)cc5c4o3)oc2c1]

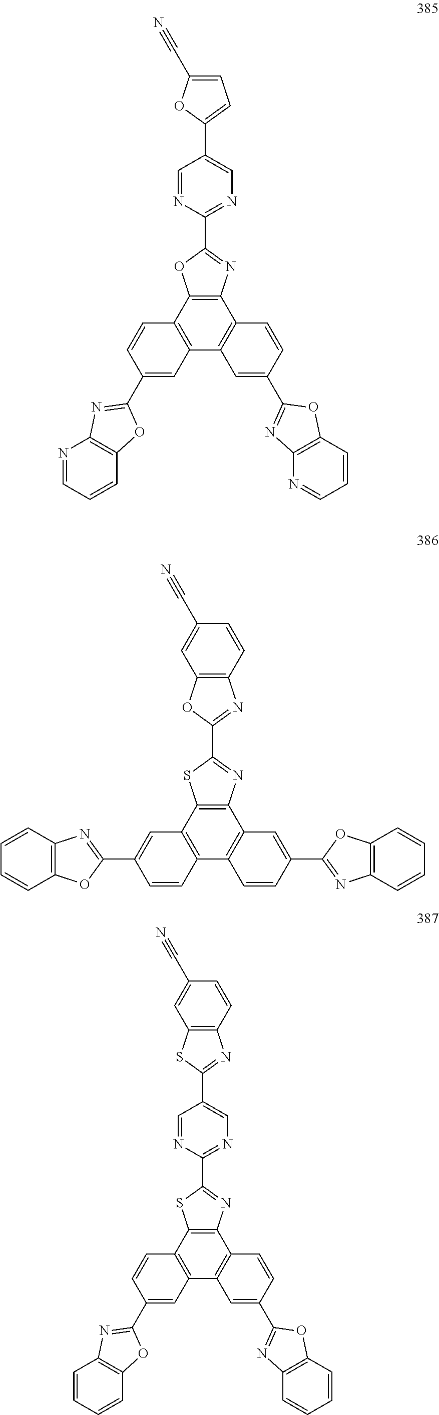 [SMILES: N#Cc1ccc(-c2cnc(-c3nc4c5ccc(-c6nc7ncccc7o6)cc5c5cc(-c6nc7ncccc7o6)ccc5c4o3)nc2)o1 ; N#Cc1ccc2nc(-c3nc4c5cc(-c6nc7ccccc7o6)ccc5c5ccc(-c6nc7ccccc7o6)cc5c4s3)oc2c1 ; N#Cc1ccc2nc(-c3cnc(-c4nc5c6ccc(-c7nc8ccccc8o7)cc6c6cc(-c7nc8ccccc8o7)ccc6c5s4)nc3)sc2c1]

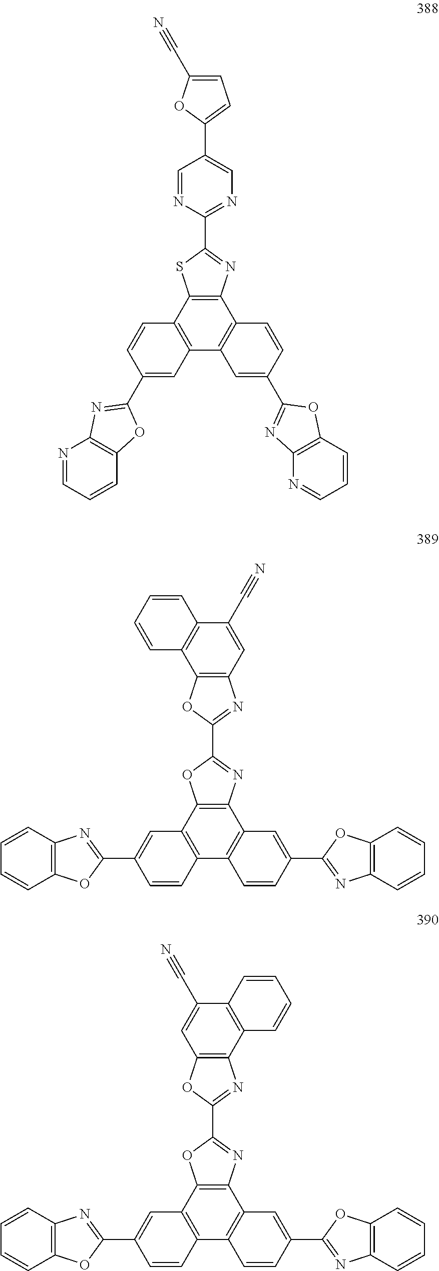 [SMILES: N#Cc1ccc(-c2cnc(-c3nc4c5ccc(-c6nc7ncccc7o6)cc5c5cc(-c6nc7ncccc7o6)ccc5c4s3)nc2)o1 ; N#Cc1cc2nc(-c3nc4c5cc(-c6nc7ccccc7o6)ccc5c5ccc(-c6nc7ccccc7o6)cc5c4o3)oc2c2ccccc12 ; N#Cc1cc2oc(-c3nc4c5cc(-c6nc7ccccc7o6)ccc5c5ccc(-c6nc7ccccc7o6)cc5c4o3)nc2c2ccccc12]

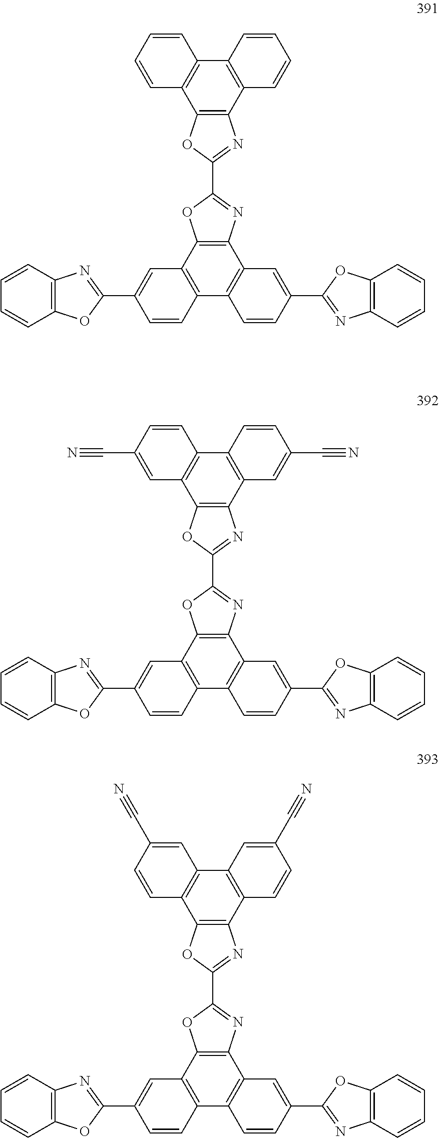 [SMILES: c1ccc2oc(-c3ccc4c5ccc(-c6nc7ccccc7o6)cc5c5oc(-c6nc7c8ccccc8c8ccccc8c7o6)nc5c4c3)nc2c1 ; N#Cc1ccc2c3ccc(C#N)cc3c3oc(-c4nc5c6cc(-c7nc8ccccc8o7)ccc6c6ccc(-c7nc8ccccc8o7)cc6c5o4)nc3c2c1 ; N#Cc1ccc2c(c1)c1cc(C#N)ccc1c1oc(-c3nc4c5cc(-c6nc7ccccc7o6)ccc5c5ccc(-c6nc7ccccc7o6)cc5c4o3)nc21]

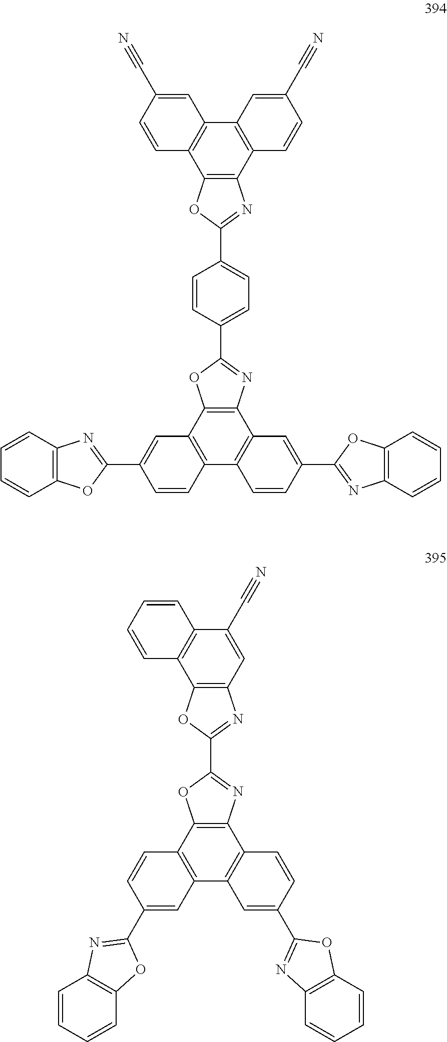 [SMILES: N#Cc1ccc2c(c1)c1cc(C#N)ccc1c1oc(-c3ccc(-c4nc5c6cc(-c7nc8ccccc8o7)ccc6c6ccc(-c7nc8ccccc8o7)cc6c5o4)cc3)nc21 ; N#Cc1cc2nc(-c3nc4c5ccc(-c6nc7ccccc7o6)cc5c5cc(-c6nc7ccccc7o6)ccc5c4o3)oc2c2ccccc12]

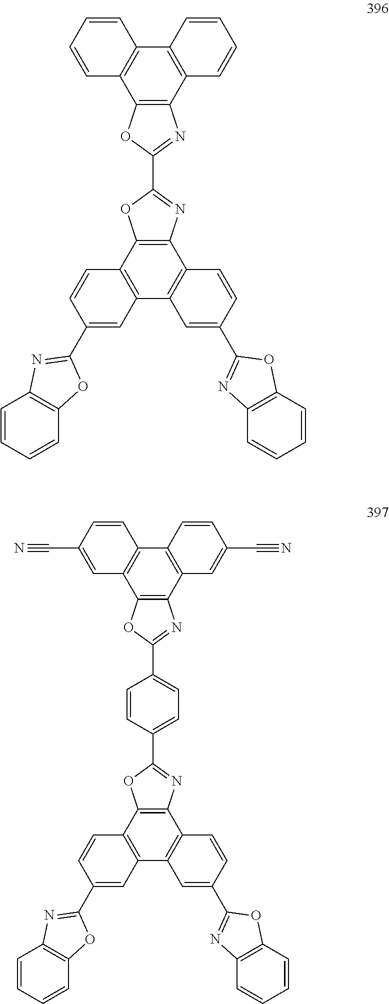 [SMILES: c1ccc2oc(-c3ccc4c(c3)c3cc(-c5nc6ccccc6o5)ccc3c3oc(-c5nc6c7ccccc7c7ccccc7c6o5)nc43)nc2c1 ; N#Cc1ccc2c3ccc(C#N)cc3c3oc(-c4ccc(-c5nc6c7ccc(-c8nc9ccccc9o8)cc7c7cc(-c8nc9ccccc9o8)ccc7c6o5)cc4)nc3c2c1]

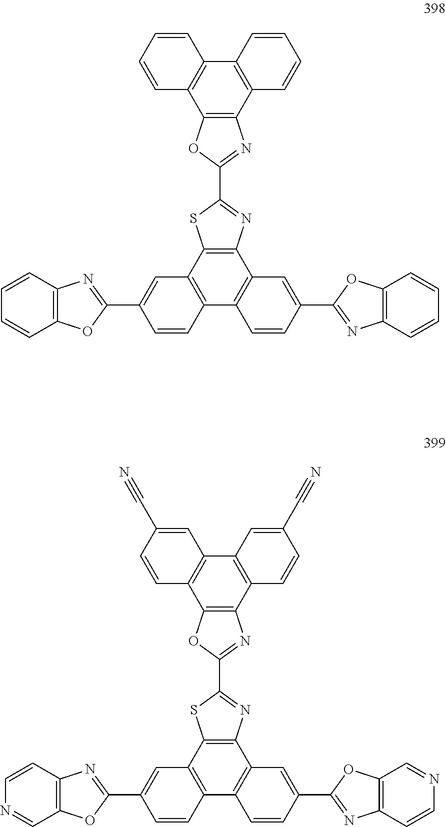 [SMILES: c1ccc2oc(-c3ccc4c5ccc(-c6nc7ccccc7o6)cc5c5sc(-c6nc7c8ccccc8c8ccccc8c7o6)nc5c4c3)nc2c1 ; N#Cc1ccc2c(c1)c1cc(C#N)ccc1c1oc(-c3nc4c5cc(-c6nc7ccncc7o6)ccc5c5ccc(-c6nc7ccncc7o6)cc5c4s3)nc21]

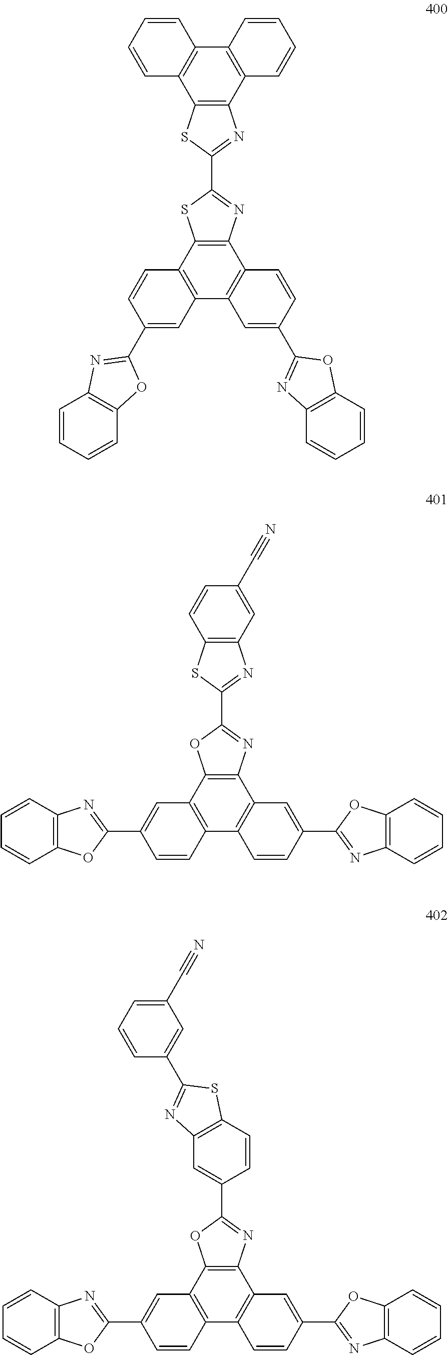 [SMILES: c1ccc2oc(-c3ccc4c(c3)c3cc(-c5nc6ccccc6o5)ccc3c3sc(-c5nc6c7ccccc7c7ccccc7c6s5)nc43)nc2c1 ; N#Cc1ccc2sc(-c3nc4c5cc(-c6nc7ccccc7o6)ccc5c5ccc(-c6nc7ccccc7o6)cc5c4o3)nc2c1 ; N#Cc1cccc(-c2nc3cc(-c4nc5c6cc(-c7nc8ccccc8o7)ccc6c6ccc(-c7nc8ccccc8o7)cc6c5o4)ccc3s2)c1]

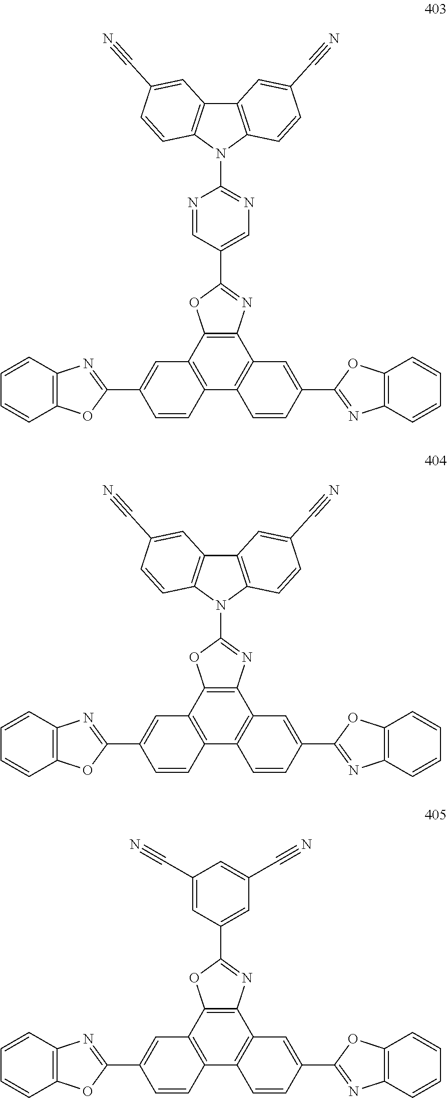 [SMILES: N#Cc1ccc2c(c1)c1cc(C#N)ccc1n2-c1ncc(-c2nc3c4cc(-c5nc6ccccc6o5)ccc4c4ccc(-c5nc6ccccc6o5)cc4c3o2)cn1 ; N#Cc1ccc2c(c1)c1cc(C#N)ccc1n2-c1nc2c3cc(-c4nc5ccccc5o4)ccc3c3ccc(-c4nc5ccccc5o4)cc3c2o1 ; N#Cc1cc(C#N)cc(-c2nc3c4cc(-c5nc6ccccc6o5)ccc4c4ccc(-c5nc6ccccc6o5)cc4c3o2)c1]

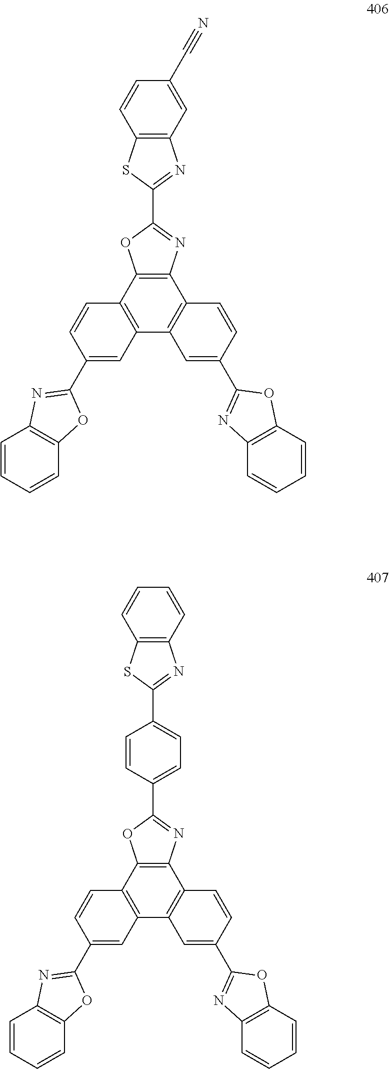 [SMILES: N#Cc1ccc2sc(-c3nc4c5ccc(-c6nc7ccccc7o6)cc5c5cc(-c6nc7ccccc7o6)ccc5c4o3)nc2c1 ; c1ccc2oc(-c3ccc4c(c3)c3cc(-c5nc6ccccc6o5)ccc3c3oc(-c5ccc(-c6nc7ccccc7s6)cc5)nc43)nc2c1]

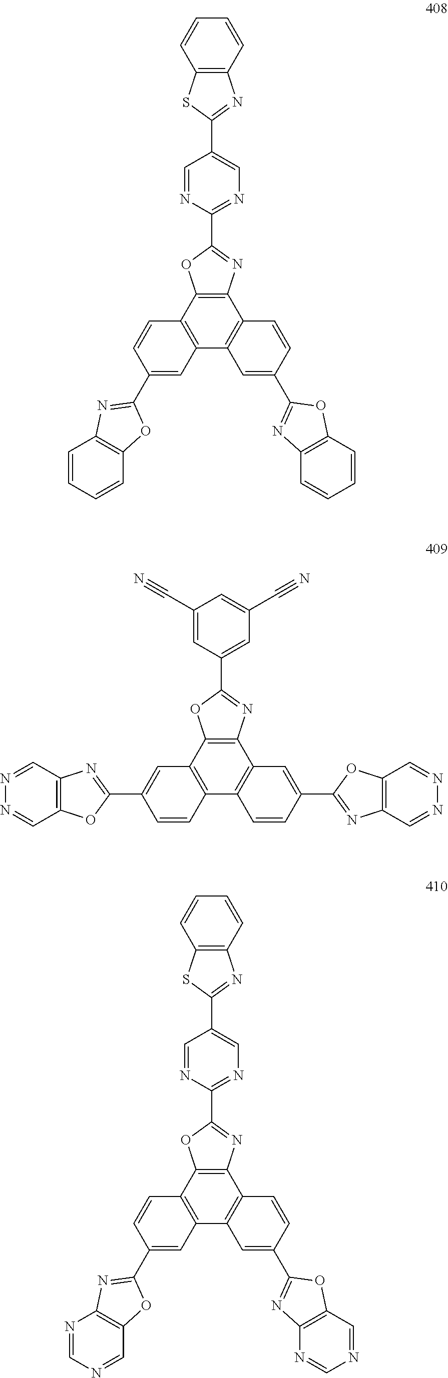 [SMILES: c1ccc2oc(-c3ccc4c(c3)c3cc(-c5nc6ccccc6o5)ccc3c3oc(-c5ncc(-c6nc7ccccc7s6)cn5)nc43)nc2c1 ; N#Cc1cc(C#N)cc(-c2nc3c4cc(-c5nc6cnncc6o5)ccc4c4ccc(-c5nc6cnncc6o5)cc4c3o2)c1 ; c1ccc2sc(-c3cnc(-c4nc5c6ccc(-c7nc8ncncc8o7)cc6c6cc(-c7nc8ncncc8o7)ccc6c5o4)nc3)nc2c1]

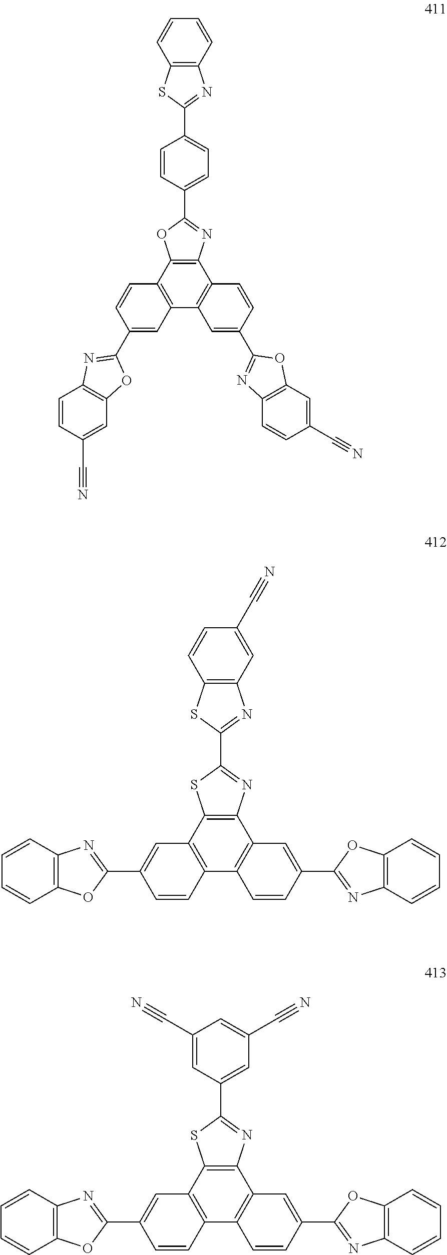 [SMILES: N#Cc1ccc2nc(-c3ccc4c(c3)c3cc(-c5nc6ccc(C#N)cc6o5)ccc3c3oc(-c5ccc(-c6nc7ccccc7s6)cc5)nc43)oc2c1 ; N#Cc1ccc2sc(-c3nc4c5cc(-c6nc7ccccc7o6)ccc5c5ccc(-c6nc7ccccc7o6)cc5c4s3)nc2c1 ; N#Cc1cc(C#N)cc(-c2nc3c4cc(-c5nc6ccccc6o5)ccc4c4ccc(-c5nc6ccccc6o5)cc4c3s2)c1]

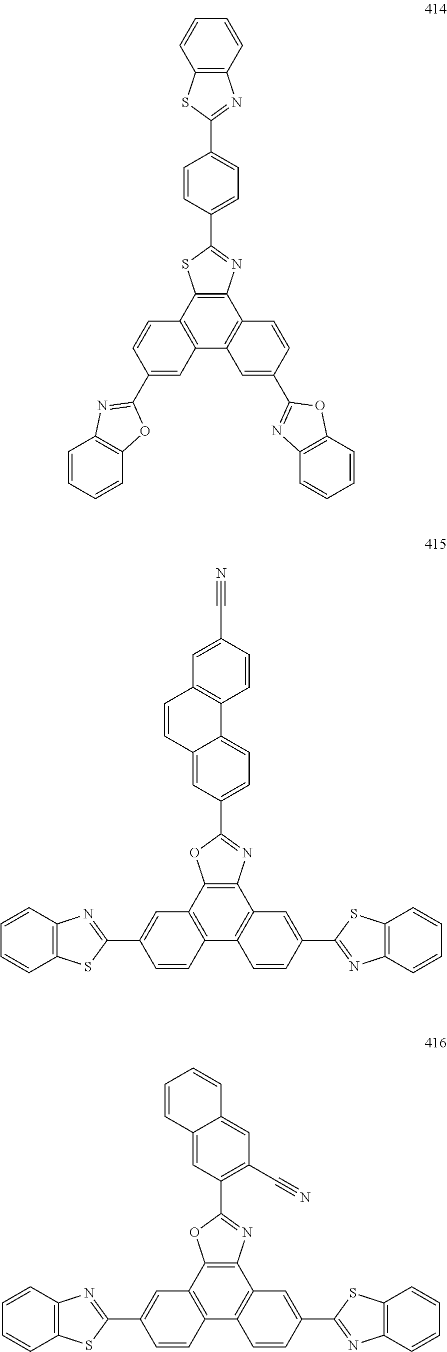 [SMILES: c1ccc2oc(-c3ccc4c(c3)c3cc(-c5nc6ccccc6o5)ccc3c3sc(-c5ccc(-c6nc7ccccc7s6)cc5)nc43)nc2c1 ; N#Cc1ccc2c(ccc3cc(-c4nc5c6cc(-c7nc8ccccc8s7)ccc6c6ccc(-c7nc8ccccc8s7)cc6c5o4)ccc32)c1 ; N#Cc1cc2ccccc2cc1-c1nc2c3cc(-c4nc5ccccc5s4)ccc3c3ccc(-c4nc5ccccc5s4)cc3c2o1]

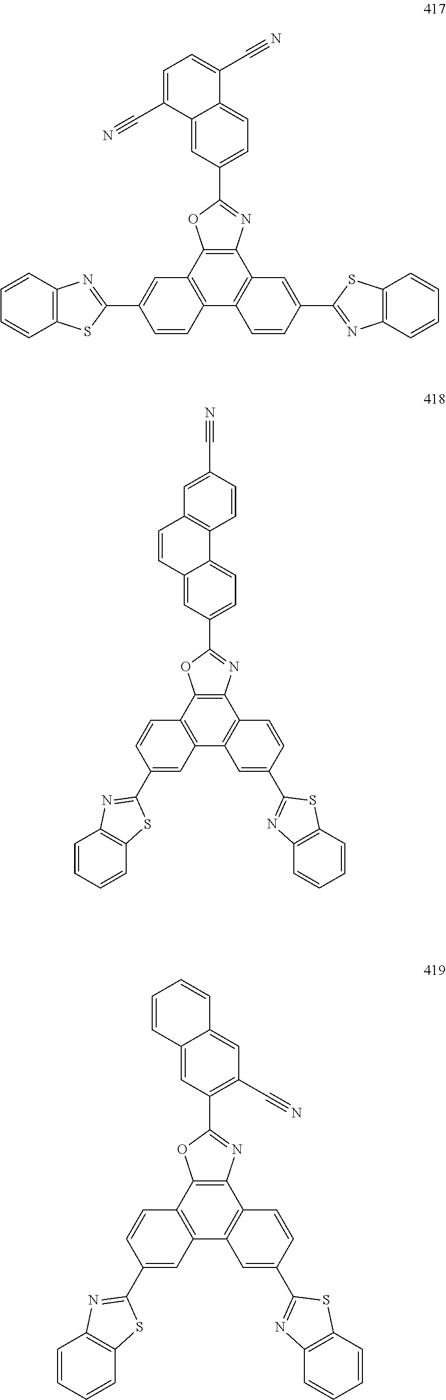 [SMILES: N#Cc1ccc(C#N)c2cc(-c3nc4c5cc(-c6nc7ccccc7s6)ccc5c5ccc(-c6nc7ccccc7s6)cc5c4o3)ccc12 ; N#Cc1ccc2c(ccc3cc(-c4nc5c6ccc(-c7nc8ccccc8s7)cc6c6cc(-c7nc8ccccc8s7)ccc6c5o4)ccc32)c1 ; N#Cc1cc2ccccc2cc1-c1nc2c3ccc(-c4nc5ccccc5s4)cc3c3cc(-c4nc5ccccc5s4)ccc3c2o1]

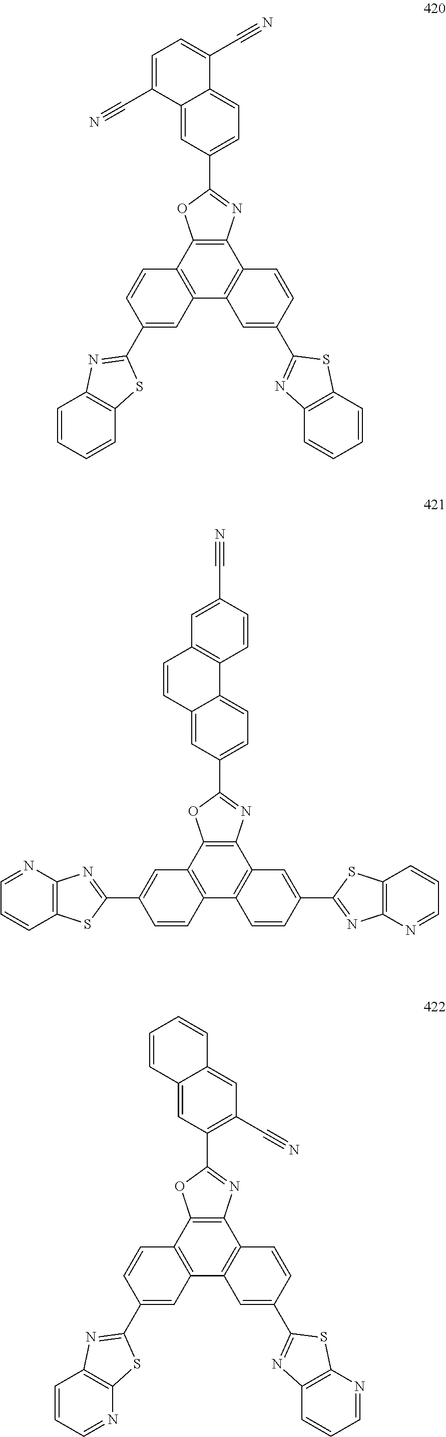 [SMILES: N#Cc1ccc(C#N)c2cc(-c3nc4c5ccc(-c6nc7ccccc7s6)cc5c5cc(-c6nc7ccccc7s6)ccc5c4o3)ccc12 ; N#Cc1ccc2c(ccc3cc(-c4nc5c6cc(-c7nc8ncccc8s7)ccc6c6ccc(-c7nc8ncccc8s7)cc6c5o4)ccc32)c1 ; N#Cc1cc2ccccc2cc1-c1nc2c3ccc(-c4nc5cccnc5s4)cc3c3cc(-c4nc5cccnc5s4)ccc3c2o1]

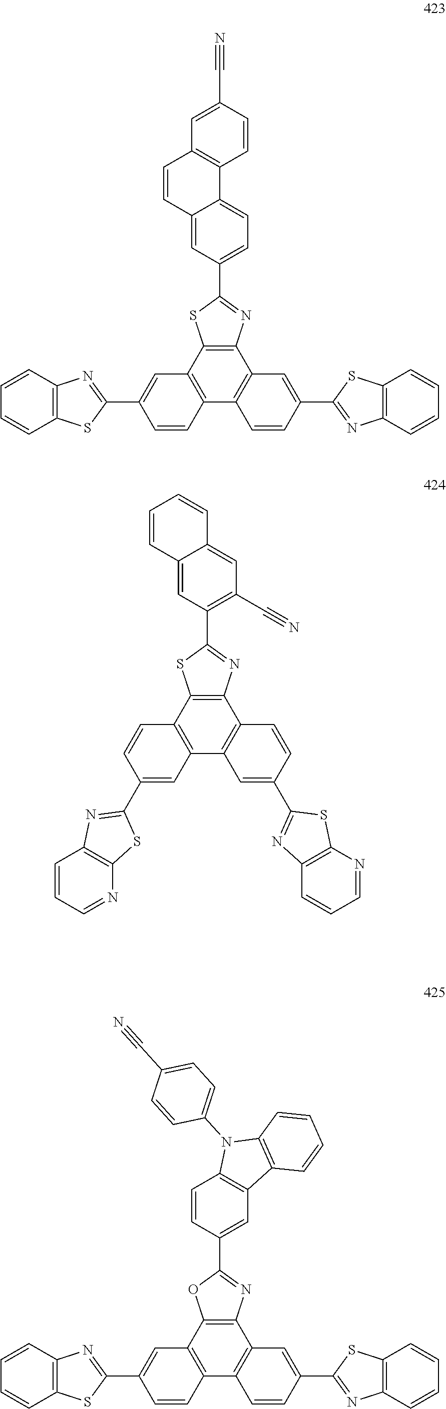 [SMILES: N#Cc1ccc2c(ccc3cc(-c4nc5c6cc(-c7nc8ccccc8s7)ccc6c6ccc(-c7nc8ccccc8s7)cc6c5s4)ccc32)c1 ; N#Cc1cc2ccccc2cc1-c1nc2c3ccc(-c4nc5cccnc5s4)cc3c3cc(-c4nc5cccnc5s4)ccc3c2s1 ; N#Cc1ccc(-n2c3ccccc3c3cc(-c4nc5c6cc(-c7nc8ccccc8s7)ccc6c6ccc(-c7nc8ccccc8s7)cc6c5o4)ccc32)cc1]

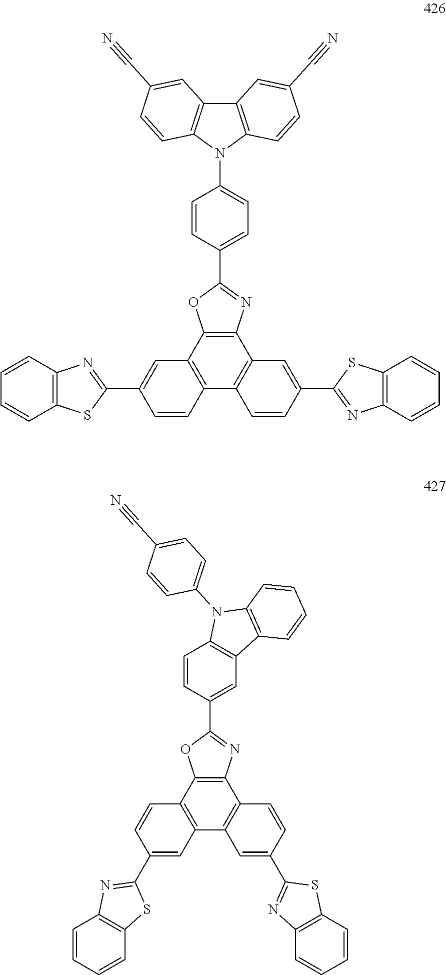 [SMILES: N#Cc1ccc2c(c1)c1cc(C#N)ccc1n2-c1ccc(-c2nc3c4cc(-c5nc6ccccc6s5)ccc4c4ccc(-c5nc6ccccc6s5)cc4c3o2)cc1 ; N#Cc1ccc(-n2c3ccccc3c3cc(-c4nc5c6ccc(-c7nc8ccccc8s7)cc6c6cc(-c7nc8ccccc8s7)ccc6c5o4)ccc32)cc1]

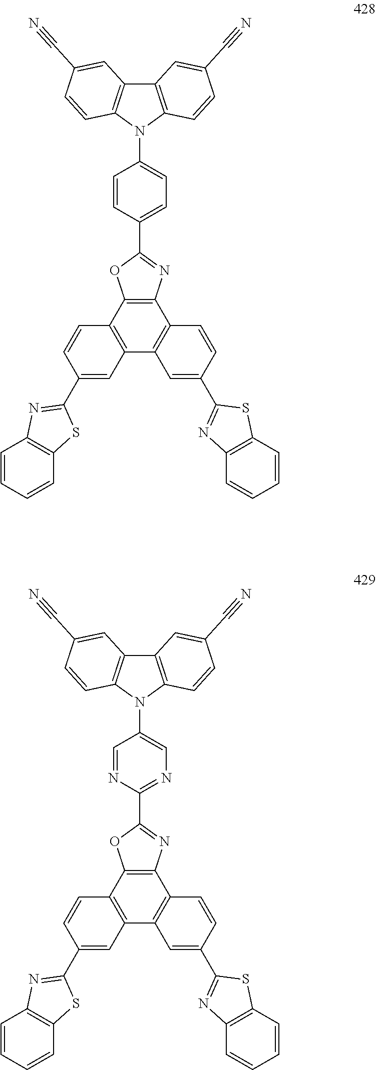 [SMILES: N#Cc1ccc2c(c1)c1cc(C#N)ccc1n2-c1ccc(-c2nc3c4ccc(-c5nc6ccccc6s5)cc4c4cc(-c5nc6ccccc6s5)ccc4c3o2)cc1 ; N#Cc1ccc2c(c1)c1cc(C#N)ccc1n2-c1cnc(-c2nc3c4ccc(-c5nc6ccccc6s5)cc4c4cc(-c5nc6ccccc6s5)ccc4c3o2)nc1]

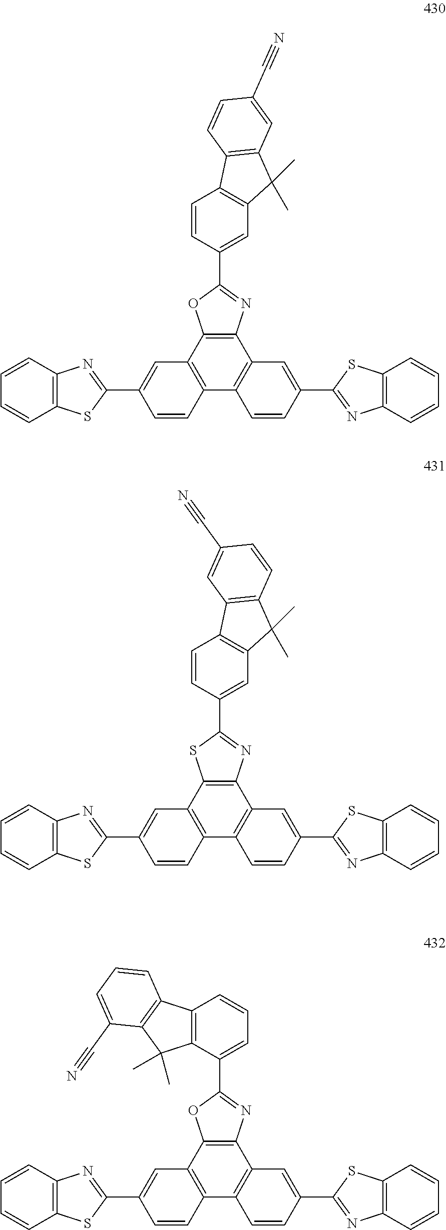 [SMILES: CC1(C)c2cc(C#N)ccc2-c2ccc(-c3nc4c5cc(-c6nc7ccccc7s6)ccc5c5ccc(-c6nc7ccccc7s6)cc5c4o3)cc21 ; CC1(C)c2ccc(C#N)cc2-c2ccc(-c3nc4c5cc(-c6nc7ccccc7s6)ccc5c5ccc(-c6nc7ccccc7s6)cc5c4s3)cc21 ; CC1(C)c2c(C#N)cccc2-c2cccc(-c3nc4c5cc(-c6nc7ccccc7s6)ccc5c5ccc(-c6nc7ccccc7s6)cc5c4o3)c21]

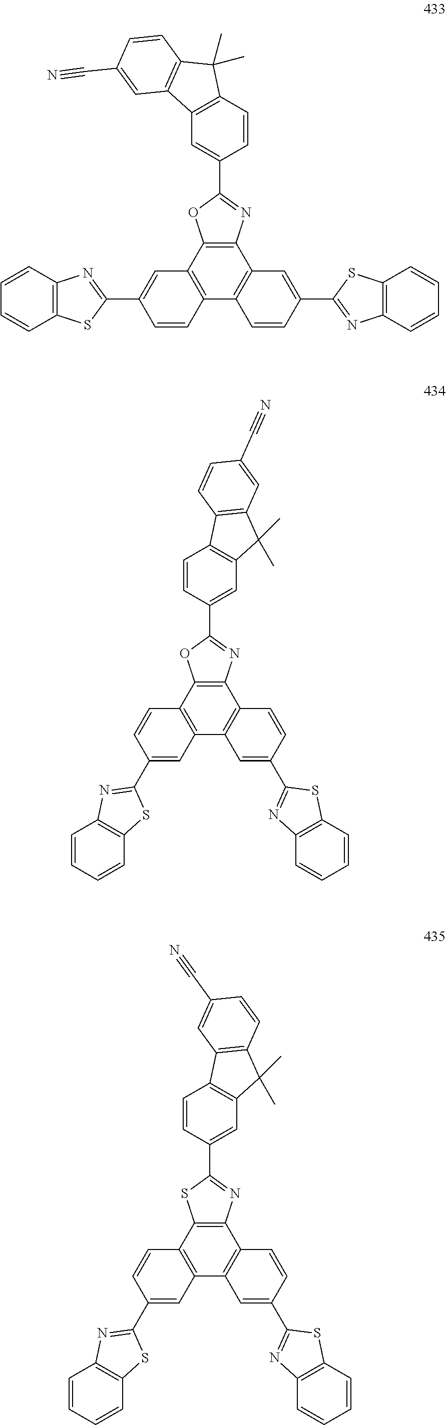 [SMILES: CC1(C)c2ccc(C#N)cc2-c2cc(-c3nc4c5cc(-c6nc7ccccc7s6)ccc5c5ccc(-c6nc7ccccc7s6)cc5c4o3)ccc21 ; CC1(C)c2cc(C#N)ccc2-c2ccc(-c3nc4c5ccc(-c6nc7ccccc7s6)cc5c5cc(-c6nc7ccccc7s6)ccc5c4o3)cc21 ; CC1(C)c2ccc(C#N)cc2-c2ccc(-c3nc4c5ccc(-c6nc7ccccc7s6)cc5c5cc(-c6nc7ccccc7s6)ccc5c4s3)cc21]

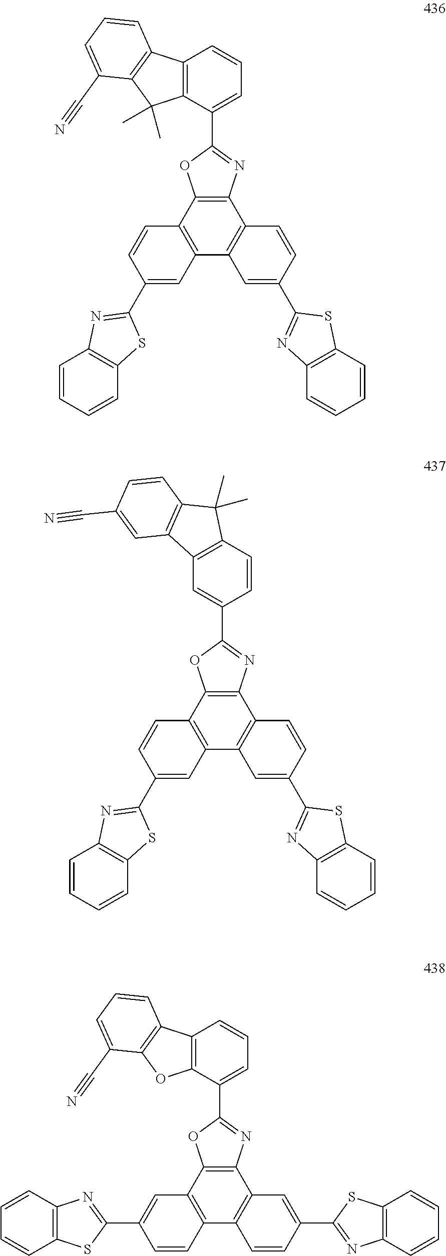 [SMILES: CC1(C)c2c(C#N)cccc2-c2cccc(-c3nc4c5ccc(-c6nc7ccccc7s6)cc5c5cc(-c6nc7ccccc7s6)ccc5c4o3)c21 ; CC1(C)c2ccc(C#N)cc2-c2cc(-c3nc4c5ccc(-c6nc7ccccc7s6)cc5c5cc(-c6nc7ccccc7s6)ccc5c4o3)ccc21 ; N#Cc1cccc2c1oc1c(-c3nc4c5cc(-c6nc7ccccc7s6)ccc5c5ccc(-c6nc7ccccc7s6)cc5c4o3)cccc12]

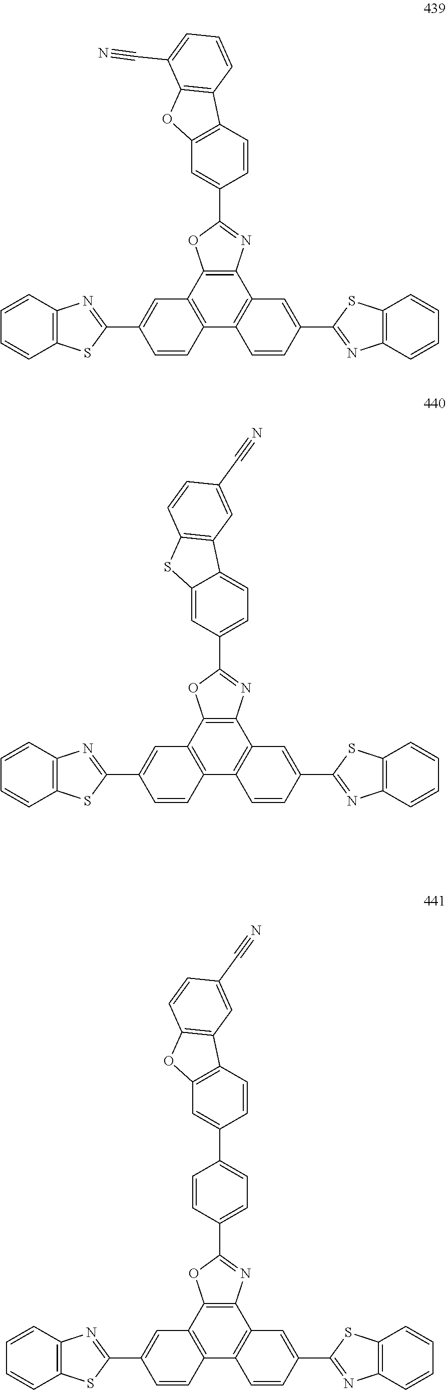 [SMILES: N#Cc1cccc2c1oc1cc(-c3nc4c5cc(-c6nc7ccccc7s6)ccc5c5ccc(-c6nc7ccccc7s6)cc5c4o3)ccc12 ; N#Cc1ccc2sc3cc(-c4nc5c6cc(-c7nc8ccccc8s7)ccc6c6ccc(-c7nc8ccccc8s7)cc6c5o4)ccc3c2c1 ; N#Cc1ccc2oc3cc(-c4ccc(-c5nc6c7cc(-c8nc9ccccc9s8)ccc7c7ccc(-c8nc9ccccc9s8)cc7c6o5)cc4)ccc3c2c1]

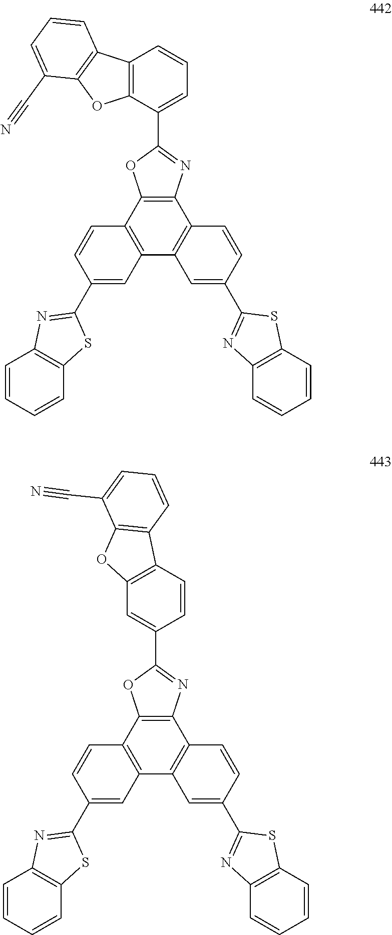 [SMILES: N#Cc1cccc2c1oc1c(-c3nc4c5ccc(-c6nc7ccccc7s6)cc5c5cc(-c6nc7ccccc7s6)ccc5c4o3)cccc12 ; N#Cc1cccc2c1oc1cc(-c3nc4c5ccc(-c6nc7ccccc7s6)cc5c5cc(-c6nc7ccccc7s6)ccc5c4o3)ccc12]

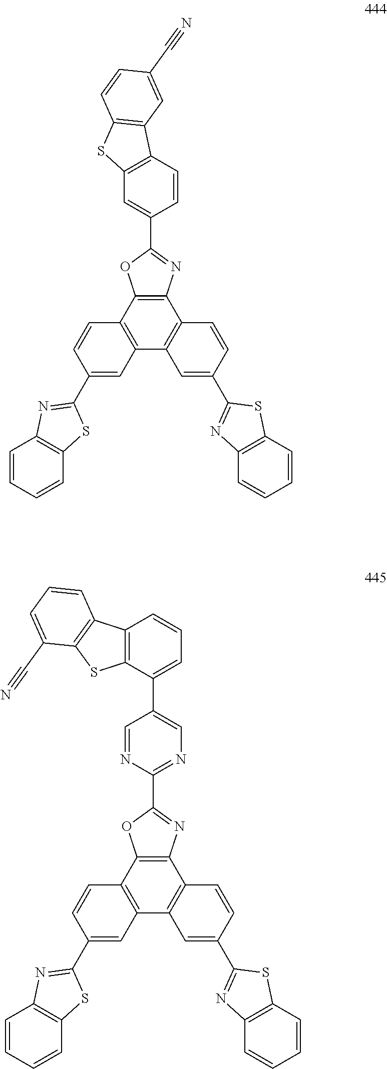 [SMILES: N#Cc1ccc2sc3cc(-c4nc5c6ccc(-c7nc8ccccc8s7)cc6c6cc(-c7nc8ccccc8s7)ccc6c5o4)ccc3c2c1 ; N#Cc1cccc2c1sc1c(-c3cnc(-c4nc5c6ccc(-c7nc8ccccc8s7)cc6c6cc(-c7nc8ccccc8s7)ccc6c5o4)nc3)cccc12]

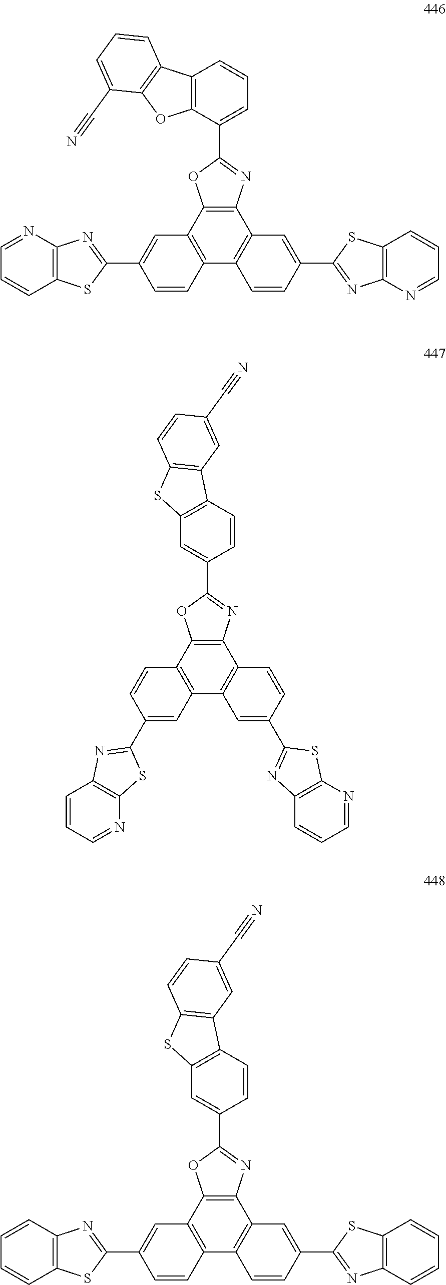 [SMILES: N#Cc1cccc2c1oc1c(-c3nc4c5cc(-c6nc7ncccc7s6)ccc5c5ccc(-c6nc7ncccc7s6)cc5c4o3)cccc12 ; N#Cc1ccc2sc3cc(-c4nc5c6ccc(-c7nc8cccnc8s7)cc6c6cc(-c7nc8cccnc8s7)ccc6c5o4)ccc3c2c1 ; N#Cc1ccc2sc3cc(-c4nc5c6cc(-c7nc8ccccc8s7)ccc6c6ccc(-c7nc8ccccc8s7)cc6c5o4)ccc3c2c1]

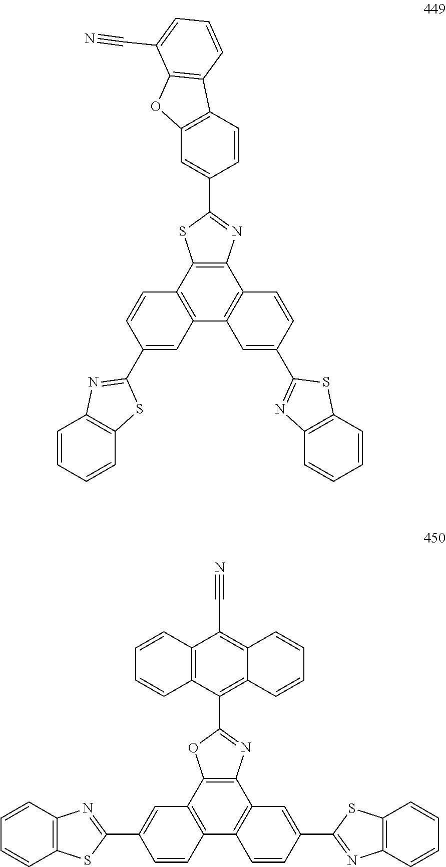 [SMILES: N#Cc1cccc2c1oc1cc(-c3nc4c5ccc(-c6nc7ccccc7s6)cc5c5cc(-c6nc7ccccc7s6)ccc5c4s3)ccc12 ; N#Cc1c2ccccc2c(-c2nc3c4cc(-c5nc6ccccc6s5)ccc4c4ccc(-c5nc6ccccc6s5)cc4c3o2)c2ccccc12]

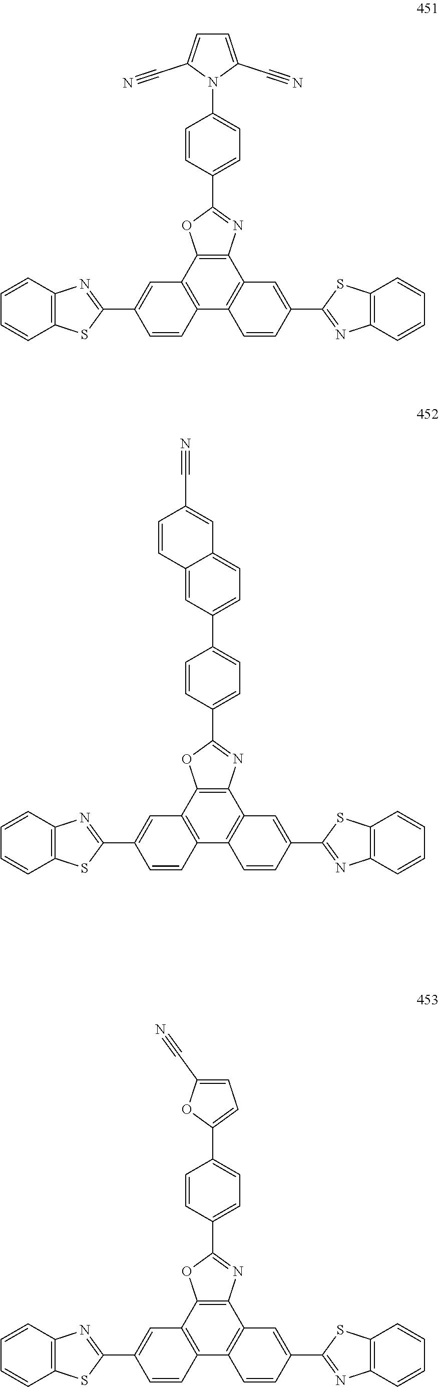 [SMILES: N#Cc1ccc(C#N)n1-c1ccc(-c2nc3c4cc(-c5nc6ccccc6s5)ccc4c4ccc(-c5nc6ccccc6s5)cc4c3o2)cc1 ; N#Cc1ccc2cc(-c3ccc(-c4nc5c6cc(-c7nc8ccccc8s7)ccc6c6ccc(-c7nc8ccccc8s7)cc6c5o4)cc3)ccc2c1 ; N#Cc1ccc(-c2ccc(-c3nc4c5cc(-c6nc7ccccc7s6)ccc5c5ccc(-c6nc7ccccc7s6)cc5c4o3)cc2)o1]

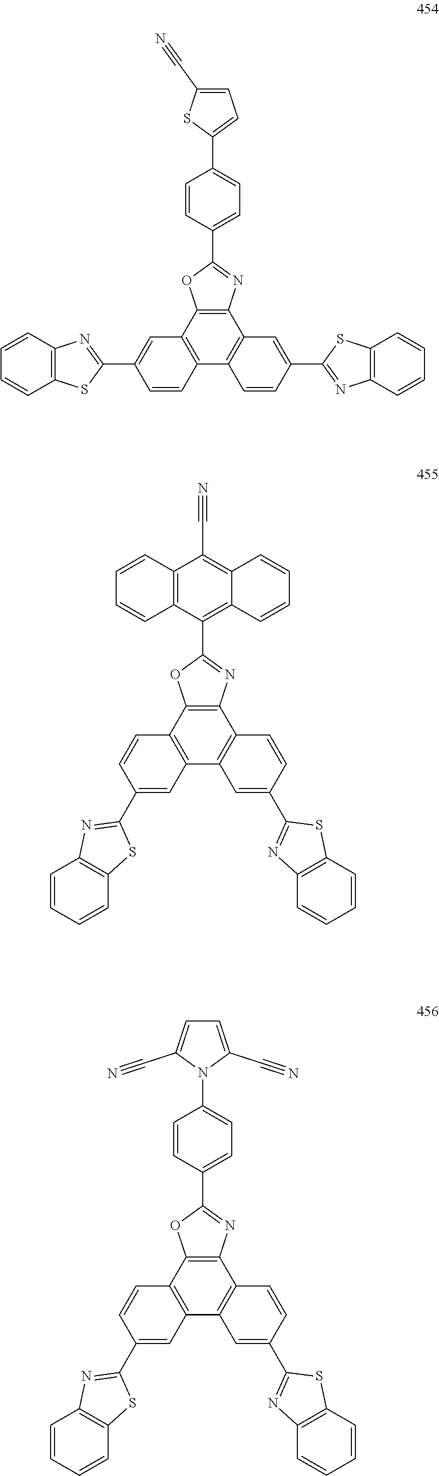 [SMILES: N#Cc1ccc(-c2ccc(-c3nc4c5cc(-c6nc7ccccc7s6)ccc5c5ccc(-c6nc7ccccc7s6)cc5c4o3)cc2)s1 ; N#Cc1c2ccccc2c(-c2nc3c4ccc(-c5nc6ccccc6s5)cc4c4cc(-c5nc6ccccc6s5)ccc4c3o2)c2ccccc12 ; N#Cc1ccc(C#N)n1-c1ccc(-c2nc3c4ccc(-c5nc6ccccc6s5)cc4c4cc(-c5nc6ccccc6s5)ccc4c3o2)cc1]

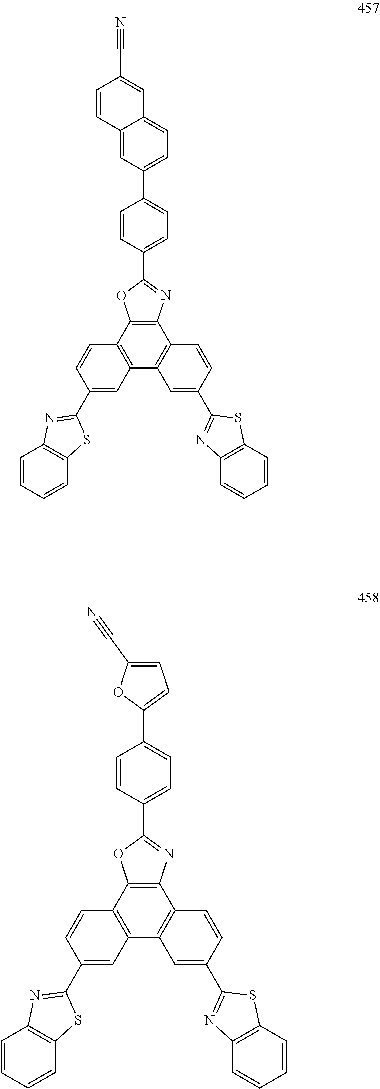 [SMILES: N#Cc1ccc2cc(-c3ccc(-c4nc5c6ccc(-c7nc8ccccc8s7)cc6c6cc(-c7nc8ccccc8s7)ccc6c5o4)cc3)ccc2c1 ; N#Cc1ccc(-c2ccc(-c3nc4c5ccc(-c6nc7ccccc7s6)cc5c5cc(-c6nc7ccccc7s6)ccc5c4o3)cc2)o1]

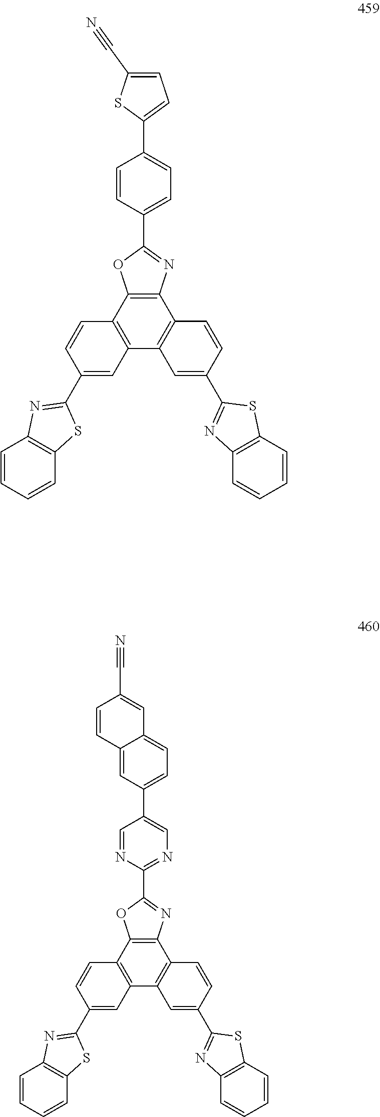 [SMILES: N#Cc1ccc(-c2ccc(-c3nc4c5ccc(-c6nc7ccccc7s6)cc5c5cc(-c6nc7ccccc7s6)ccc5c4o3)cc2)s1 ; N#Cc1ccc2cc(-c3cnc(-c4nc5c6ccc(-c7nc8ccccc8s7)cc6c6cc(-c7nc8ccccc8s7)ccc6c5o4)nc3)ccc2c1]

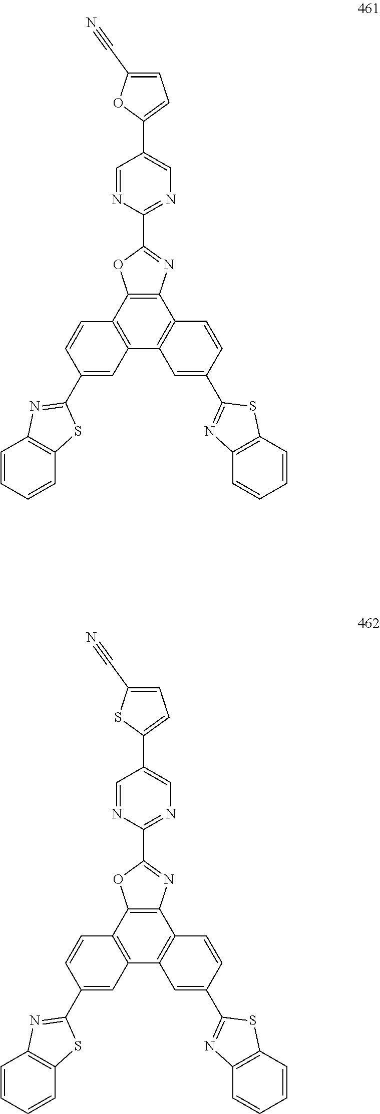 [SMILES: N#Cc1ccc(-c2cnc(-c3nc4c5ccc(-c6nc7ccccc7s6)cc5c5cc(-c6nc7ccccc7s6)ccc5c4o3)nc2)o1 ; N#Cc1ccc(-c2cnc(-c3nc4c5ccc(-c6nc7ccccc7s6)cc5c5cc(-c6nc7ccccc7s6)ccc5c4o3)nc2)s1]

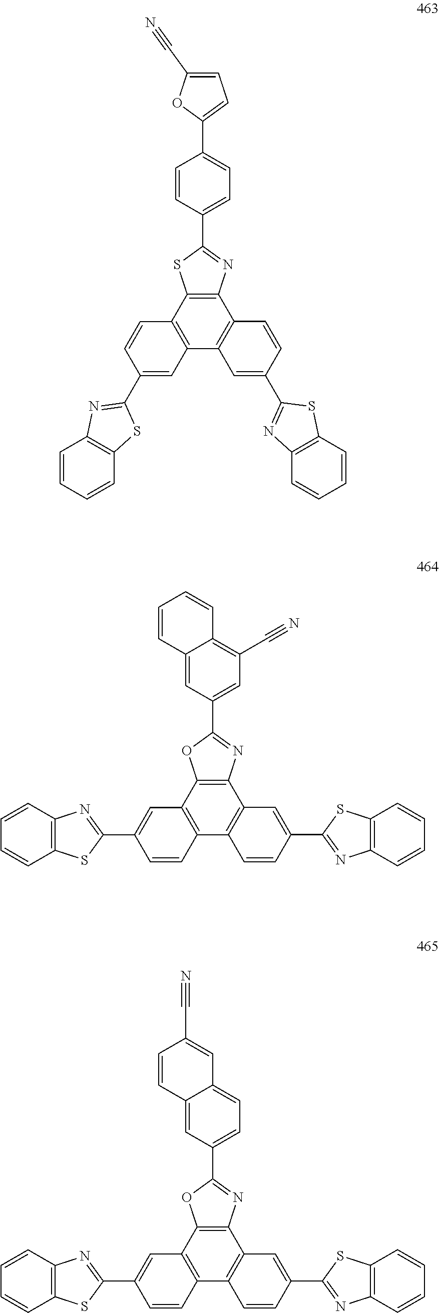 [SMILES: N#Cc1ccc(-c2ccc(-c3nc4c5ccc(-c6nc7ccccc7s6)cc5c5cc(-c6nc7ccccc7s6)ccc5c4s3)cc2)o1 ; N#Cc1cc(-c2nc3c4cc(-c5nc6ccccc6s5)ccc4c4ccc(-c5nc6ccccc6s5)cc4c3o2)cc2ccccc12 ; N#Cc1ccc2cc(-c3nc4c5cc(-c6nc7ccccc7s6)ccc5c5ccc(-c6nc7ccccc7s6)cc5c4o3)ccc2c1]

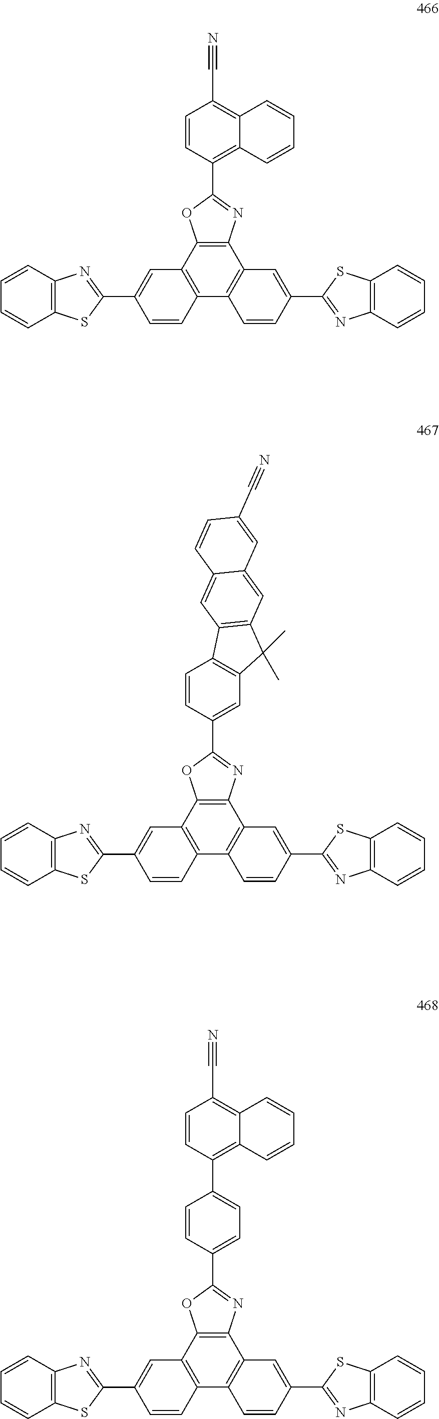 [SMILES: N#Cc1ccc(-c2nc3c4cc(-c5nc6ccccc6s5)ccc4c4ccc(-c5nc6ccccc6s5)cc4c3o2)c2ccccc12 ; CC1(C)c2cc(-c3nc4c5cc(-c6nc7ccccc7s6)ccc5c5ccc(-c6nc7ccccc7s6)cc5c4o3)ccc2-c2cc3ccc(C#N)cc3cc21 ; N#Cc1ccc(-c2ccc(-c3nc4c5cc(-c6nc7ccccc7s6)ccc5c5ccc(-c6nc7ccccc7s6)cc5c4o3)cc2)c2ccccc12]

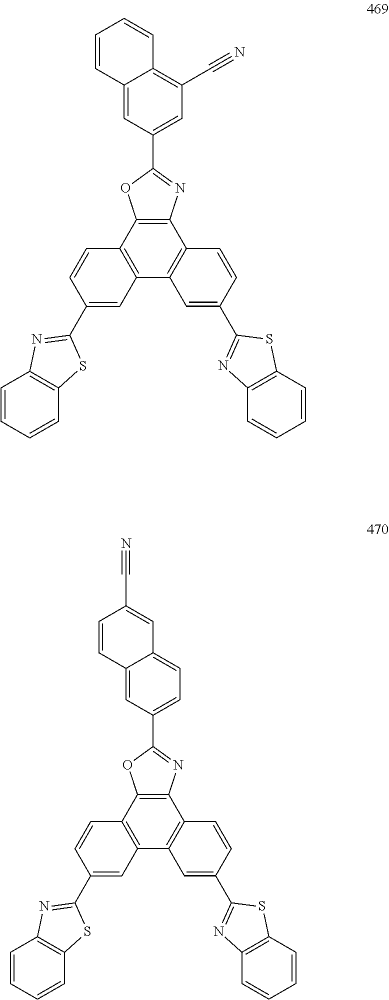 [SMILES: N#Cc1cc(-c2nc3c4ccc(-c5nc6ccccc6s5)cc4c4cc(-c5nc6ccccc6s5)ccc4c3o2)cc2ccccc12 ; N#Cc1ccc2cc(-c3nc4c5ccc(-c6nc7ccccc7s6)cc5c5cc(-c6nc7ccccc7s6)ccc5c4o3)ccc2c1]

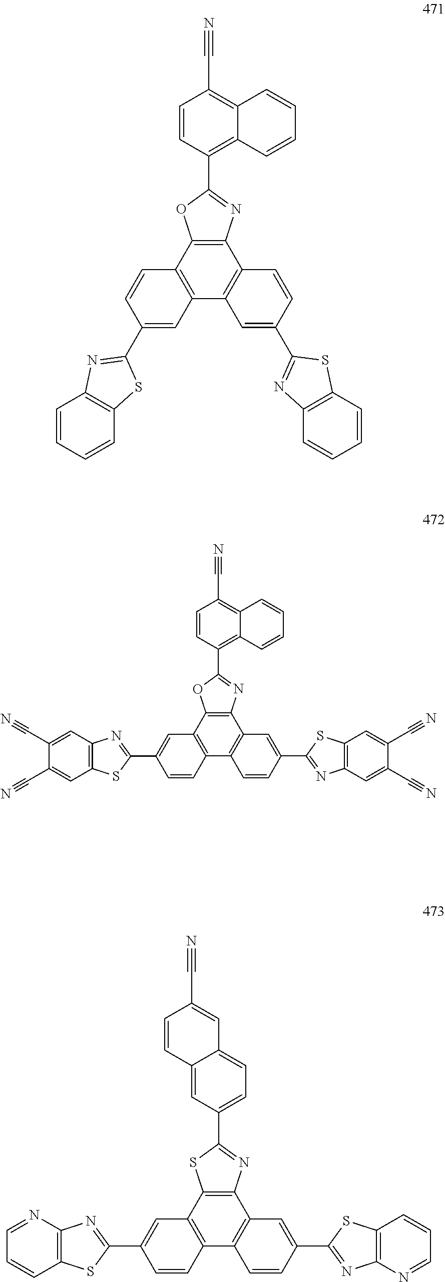 [SMILES: N#Cc1ccc(-c2nc3c4ccc(-c5nc6ccccc6s5)cc4c4cc(-c5nc6ccccc6s5)ccc4c3o2)c2ccccc12 ; N#Cc1cc2nc(-c3ccc4c5ccc(-c6nc7cc(C#N)c(C#N)cc7s6)cc5c5oc(-c6ccc(C#N)c7ccccc67)nc5c4c3)sc2cc1C#N ; N#Cc1ccc2cc(-c3nc4c5cc(-c6nc7ncccc7s6)ccc5c5ccc(-c6nc7ncccc7s6)cc5c4s3)ccc2c1]

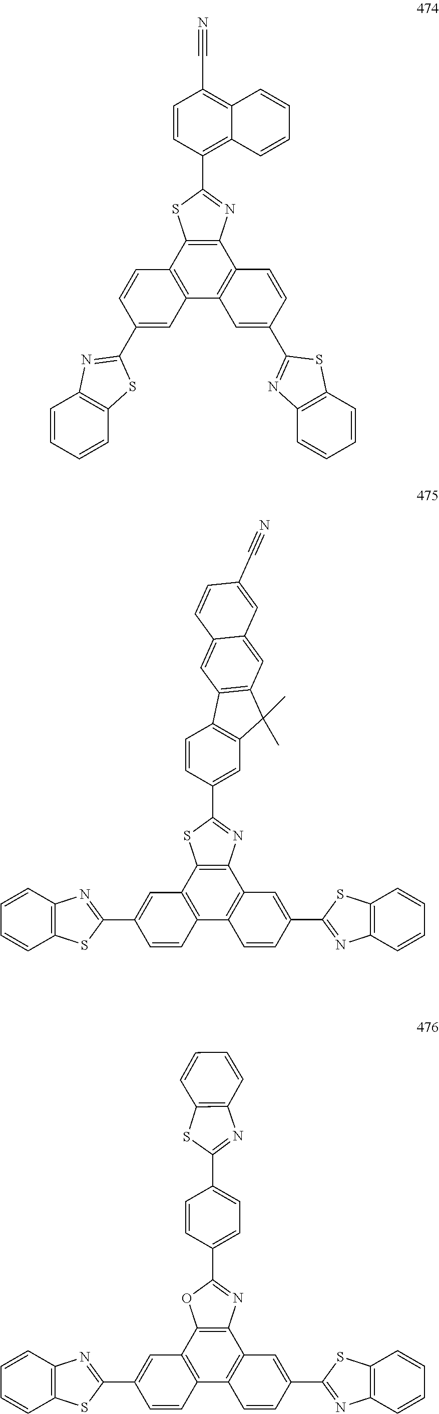 [SMILES: N#Cc1ccc(-c2nc3c4ccc(-c5nc6ccccc6s5)cc4c4cc(-c5nc6ccccc6s5)ccc4c3s2)c2ccccc12 ; CC1(C)c2cc(-c3nc4c5cc(-c6nc7ccccc7s6)ccc5c5ccc(-c6nc7ccccc7s6)cc5c4s3)ccc2-c2cc3ccc(C#N)cc3cc21 ; c1ccc2sc(-c3ccc(-c4nc5c6cc(-c7nc8ccccc8s7)ccc6c6ccc(-c7nc8ccccc8s7)cc6c5o4)cc3)nc2c1]

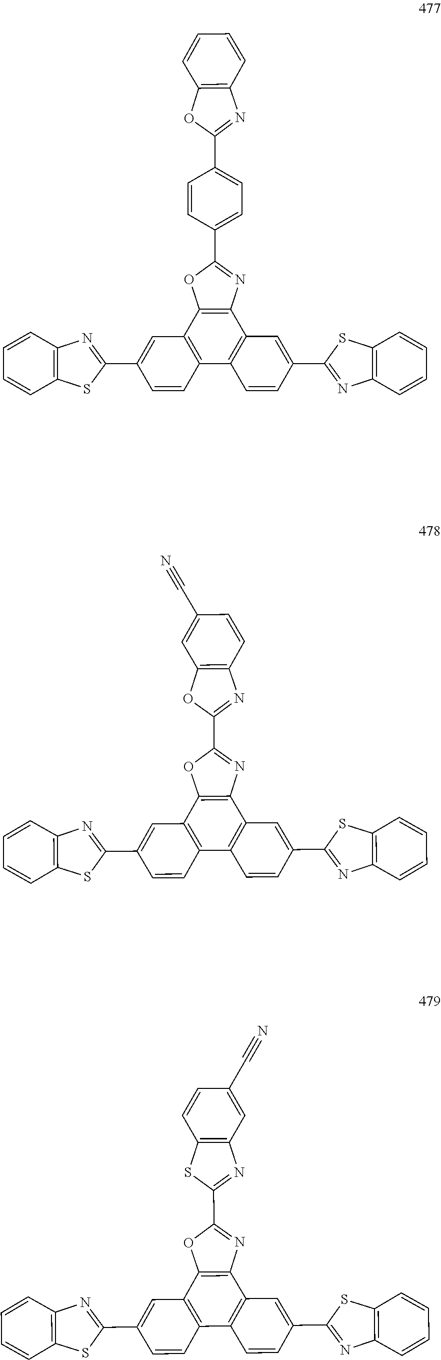 [SMILES: c1ccc2oc(-c3ccc(-c4nc5c6cc(-c7nc8ccccc8s7)ccc6c6ccc(-c7nc8ccccc8s7)cc6c5o4)cc3)nc2c1 ; N#Cc1ccc2nc(-c3nc4c5cc(-c6nc7ccccc7s6)ccc5c5ccc(-c6nc7ccccc7s6)cc5c4o3)oc2c1 ; N#Cc1ccc2sc(-c3nc4c5cc(-c6nc7ccccc7s6)ccc5c5ccc(-c6nc7ccccc7s6)cc5c4o3)nc2c1]

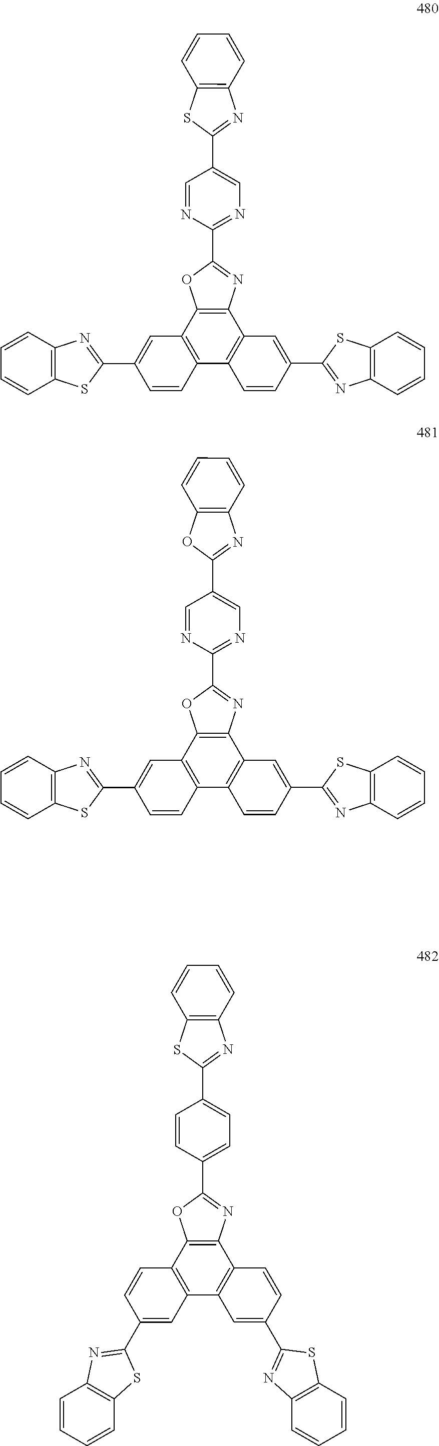 [SMILES: c1ccc2sc(-c3cnc(-c4nc5c6cc(-c7nc8ccccc8s7)ccc6c6ccc(-c7nc8ccccc8s7)cc6c5o4)nc3)nc2c1 ; c1ccc2oc(-c3cnc(-c4nc5c6cc(-c7nc8ccccc8s7)ccc6c6ccc(-c7nc8ccccc8s7)cc6c5o4)nc3)nc2c1 ; c1ccc2sc(-c3ccc(-c4nc5c6ccc(-c7nc8ccccc8s7)cc6c6cc(-c7nc8ccccc8s7)ccc6c5o4)cc3)nc2c1]

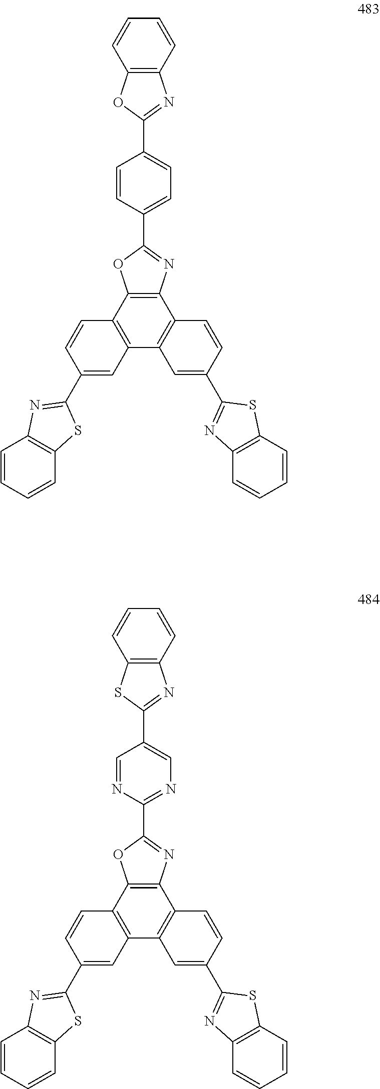 [SMILES: c1ccc2oc(-c3ccc(-c4nc5c6ccc(-c7nc8ccccc8s7)cc6c6cc(-c7nc8ccccc8s7)ccc6c5o4)cc3)nc2c1 ; c1ccc2sc(-c3cnc(-c4nc5c6ccc(-c7nc8ccccc8s7)cc6c6cc(-c7nc8ccccc8s7)ccc6c5o4)nc3)nc2c1]

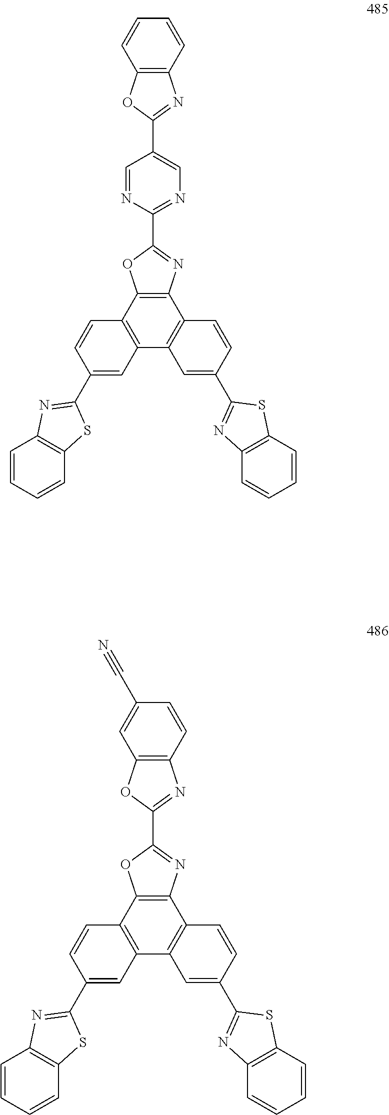 [SMILES: c1ccc2oc(-c3cnc(-c4nc5c6ccc(-c7nc8ccccc8s7)cc6c6cc(-c7nc8ccccc8s7)ccc6c5o4)nc3)nc2c1 ; N#Cc1ccc2nc(-c3nc4c5ccc(-c6nc7ccccc7s6)cc5c5cc(-c6nc7ccccc7s6)ccc5c4o3)oc2c1]

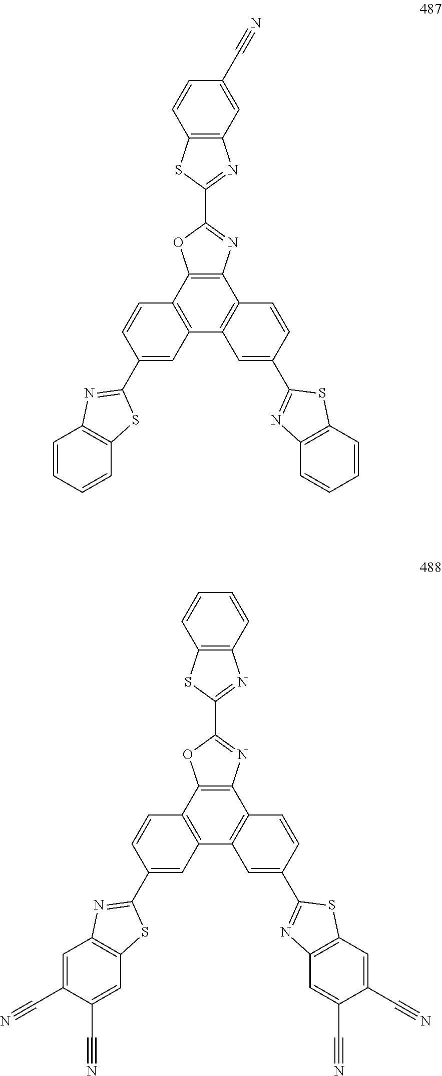 [SMILES: N#Cc1ccc2sc(-c3nc4c5ccc(-c6nc7ccccc7s6)cc5c5cc(-c6nc7ccccc7s6)ccc5c4o3)nc2c1 ; N#Cc1cc2nc(-c3ccc4c(c3)c3cc(-c5nc6cc(C#N)c(C#N)cc6s5)ccc3c3oc(-c5nc6ccccc6s5)nc43)sc2cc1C#N]

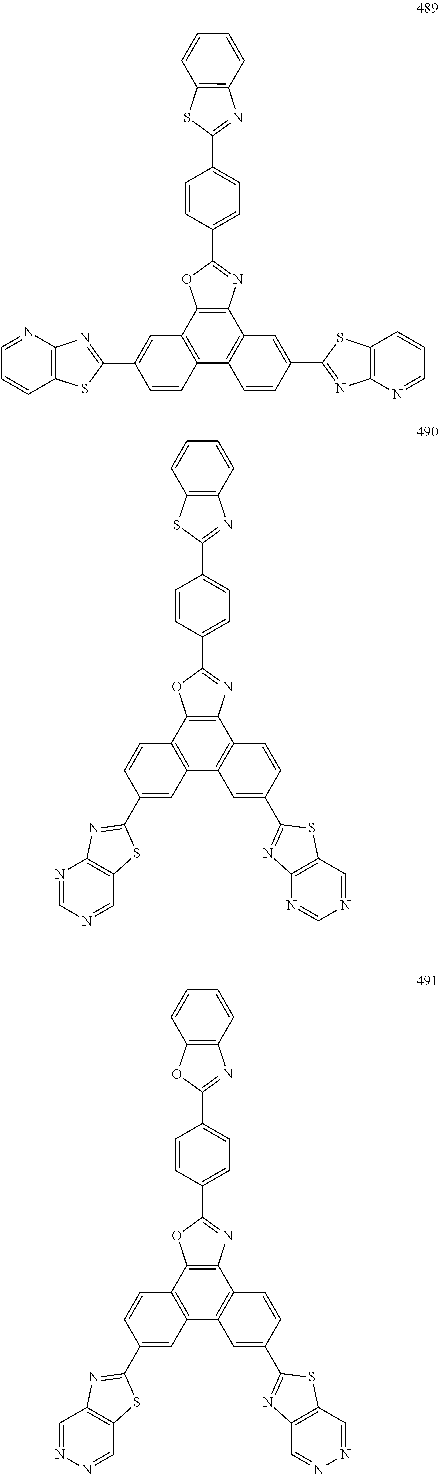 [SMILES: c1ccc2sc(-c3ccc(-c4nc5c6cc(-c7nc8ncccc8s7)ccc6c6ccc(-c7nc8ncccc8s7)cc6c5o4)cc3)nc2c1 ; c1ccc2sc(-c3ccc(-c4nc5c6ccc(-c7nc8ncncc8s7)cc6c6cc(-c7nc8ncncc8s7)ccc6c5o4)cc3)nc2c1 ; c1ccc2oc(-c3ccc(-c4nc5c6ccc(-c7nc8cnncc8s7)cc6c6cc(-c7nc8cnncc8s7)ccc6c5o4)cc3)nc2c1]

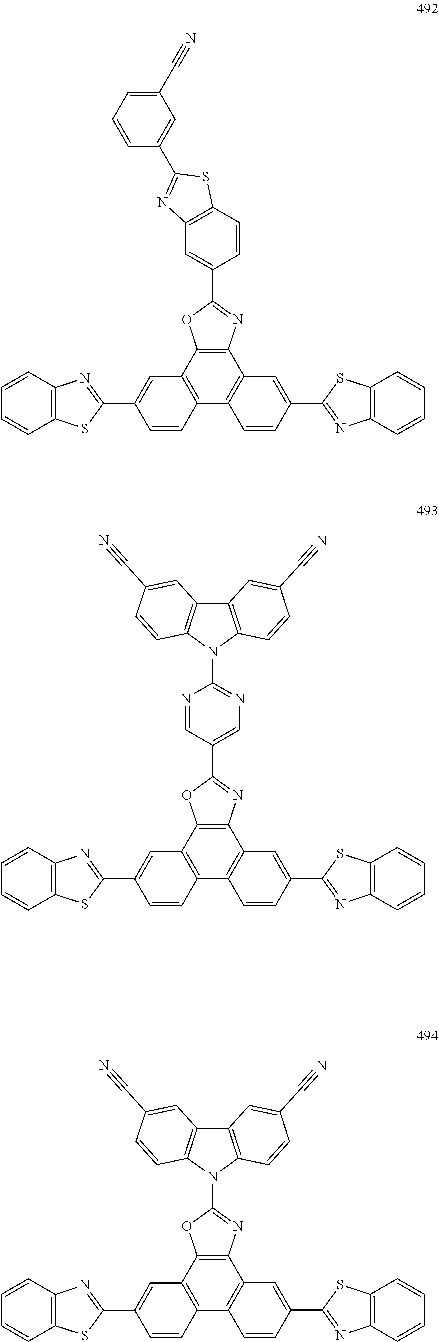 [SMILES: N#Cc1cccc(-c2nc3cc(-c4nc5c6cc(-c7nc8ccccc8s7)ccc6c6ccc(-c7nc8ccccc8s7)cc6c5o4)ccc3s2)c1 ; N#Cc1ccc2c(c1)c1cc(C#N)ccc1n2-c1ncc(-c2nc3c4cc(-c5nc6ccccc6s5)ccc4c4ccc(-c5nc6ccccc6s5)cc4c3o2)cn1 ; N#Cc1ccc2c(c1)c1cc(C#N)ccc1n2-c1nc2c3cc(-c4nc5ccccc5s4)ccc3c3ccc(-c4nc5ccccc5s4)cc3c2o1]

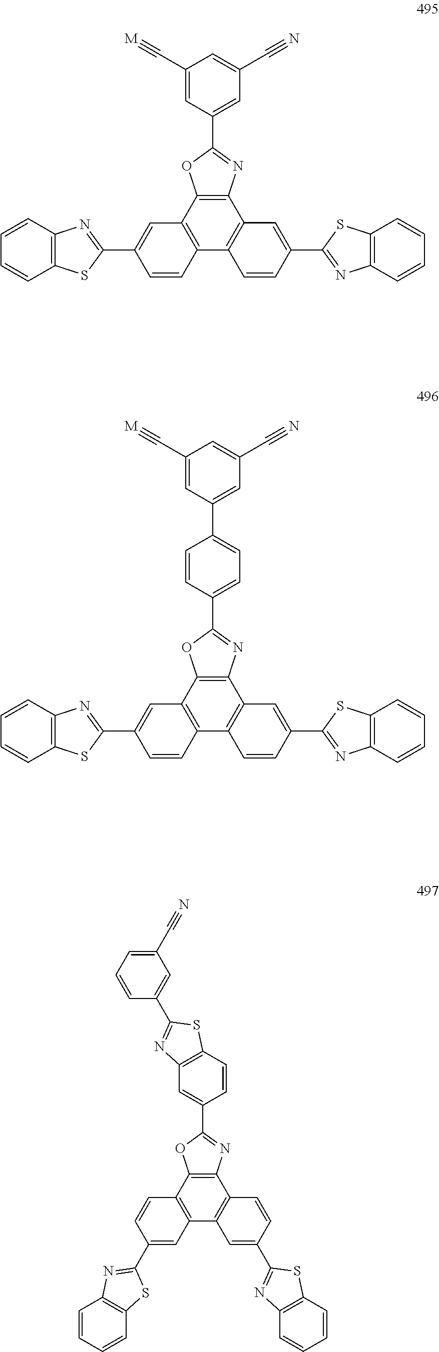

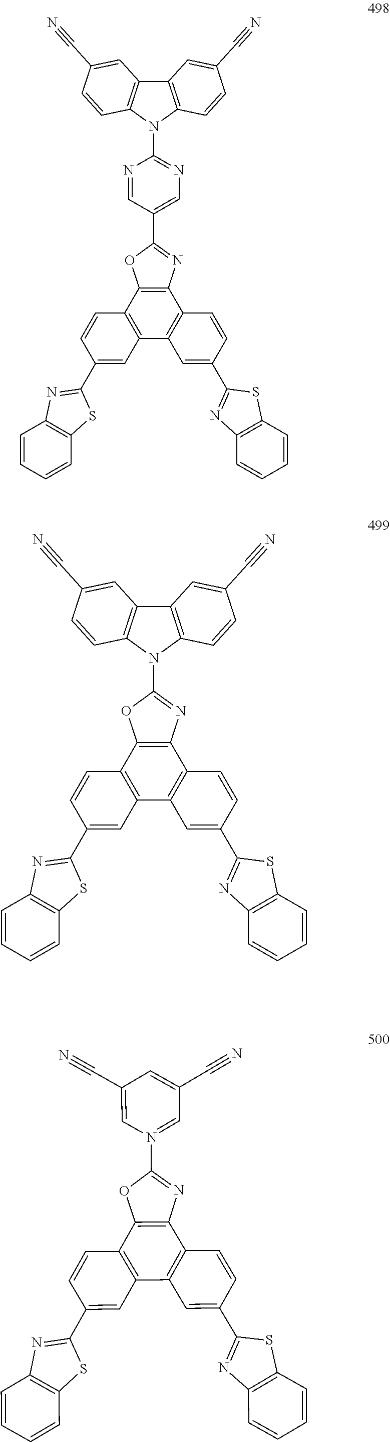

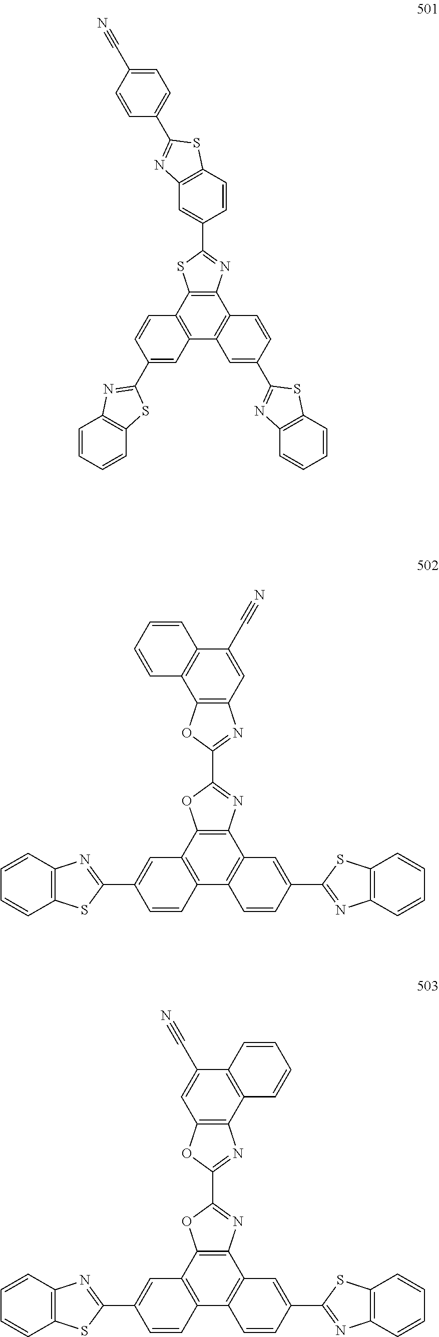 [SMILES: N#Cc1ccc(-c2nc3cc(-c4nc5c6ccc(-c7nc8ccccc8s7)cc6c6cc(-c7nc8ccccc8s7)ccc6c5s4)ccc3s2)cc1 ; N#Cc1cc2nc(-c3nc4c5cc(-c6nc7ccccc7s6)ccc5c5ccc(-c6nc7ccccc7s6)cc5c4o3)oc2c2ccccc12 ; N#Cc1cc2oc(-c3nc4c5cc(-c6nc7ccccc7s6)ccc5c5ccc(-c6nc7ccccc7s6)cc5c4o3)nc2c2ccccc12]

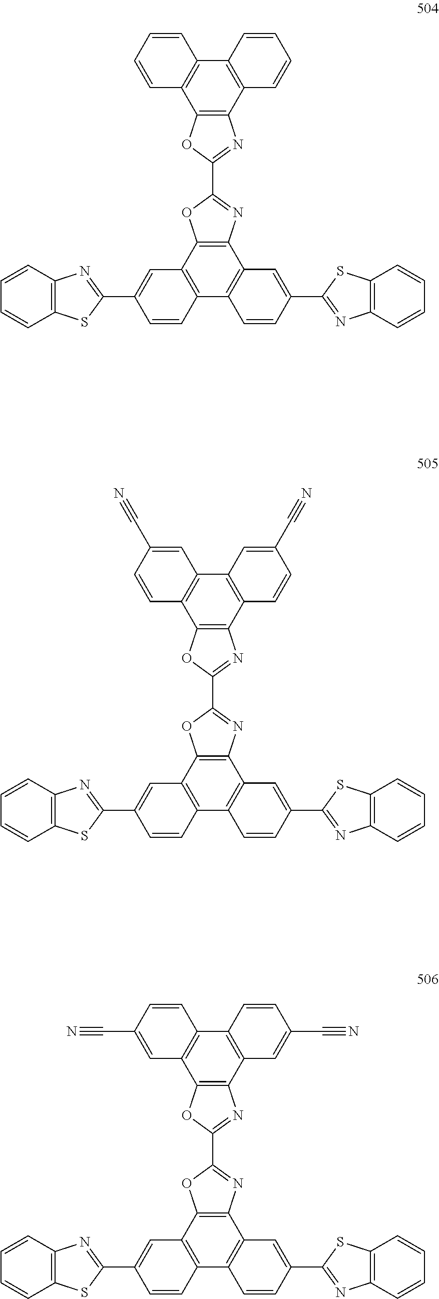 [SMILES: c1ccc2sc(-c3ccc4c5ccc(-c6nc7ccccc7s6)cc5c5oc(-c6nc7c8ccccc8c8ccccc8c7o6)nc5c4c3)nc2c1 ; N#Cc1ccc2c(c1)c1cc(C#N)ccc1c1oc(-c3nc4c5cc(-c6nc7ccccc7s6)ccc5c5ccc(-c6nc7ccccc7s6)cc5c4o3)nc21 ; N#Cc1ccc2c3ccc(C#N)cc3c3oc(-c4nc5c6cc(-c7nc8ccccc8s7)ccc6c6ccc(-c7nc8ccccc8s7)cc6c5o4)nc3c2c1]

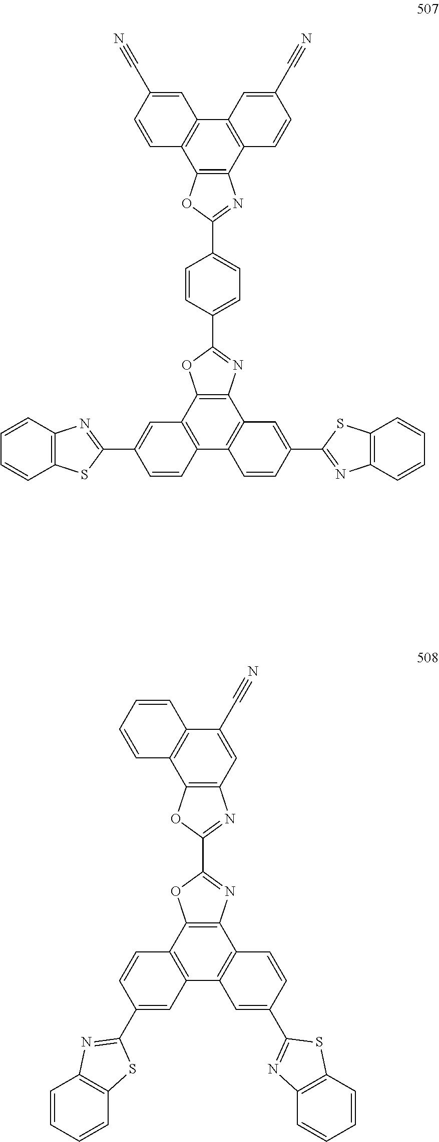 [SMILES: N#Cc1ccc2c(c1)c1cc(C#N)ccc1c1oc(-c3ccc(-c4nc5c6cc(-c7nc8ccccc8s7)ccc6c6ccc(-c7nc8ccccc8s7)cc6c5o4)cc3)nc21 ; N#Cc1cc2nc(-c3nc4c5ccc(-c6nc7ccccc7s6)cc5c5cc(-c6nc7ccccc7s6)ccc5c4o3)oc2c2ccccc12]

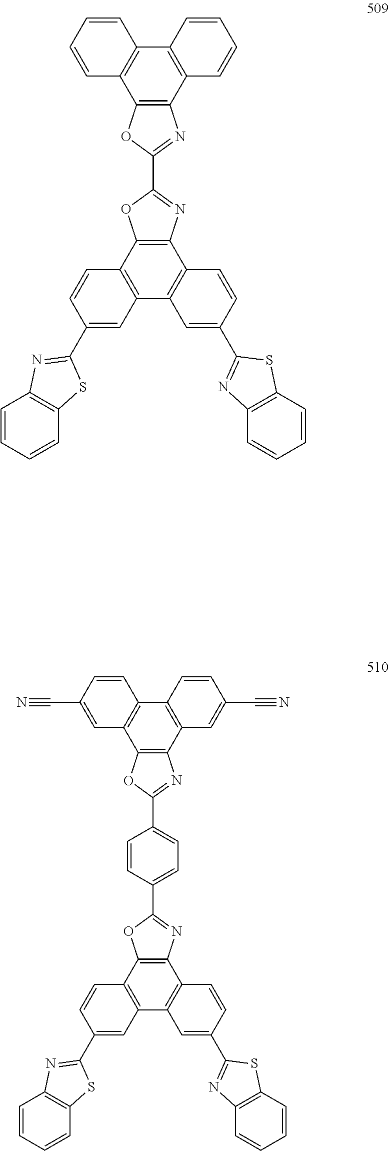 [SMILES: c1ccc2sc(-c3ccc4c(c3)c3cc(-c5nc6ccccc6s5)ccc3c3oc(-c5nc6c7ccccc7c7ccccc7c6o5)nc43)nc2c1 ; N#Cc1ccc2c3ccc(C#N)cc3c3oc(-c4ccc(-c5nc6c7ccc(-c8nc9ccccc9s8)cc7c7cc(-c8nc9ccccc9s8)ccc7c6o5)cc4)nc3c2c1]

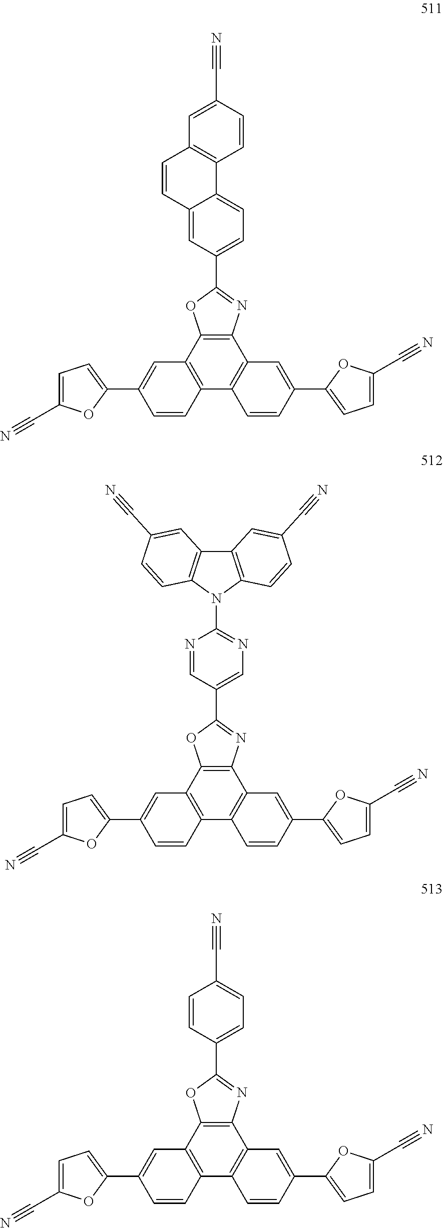 [SMILES: N#Cc1ccc2c(ccc3cc(-c4nc5c6cc(-c7ccc(C#N)o7)ccc6c6ccc(-c7ccc(C#N)o7)cc6c5o4)ccc32)c1 ; N#Cc1ccc2c(c1)c1cc(C#N)ccc1n2-c1ncc(-c2nc3c4cc(-c5ccc(C#N)o5)ccc4c4ccc(-c5ccc(C#N)o5)cc4c3o2)cn1 ; N#Cc1ccc(-c2nc3c4cc(-c5ccc(C#N)o5)ccc4c4ccc(-c5ccc(C#N)o5)cc4c3o2)cc1]

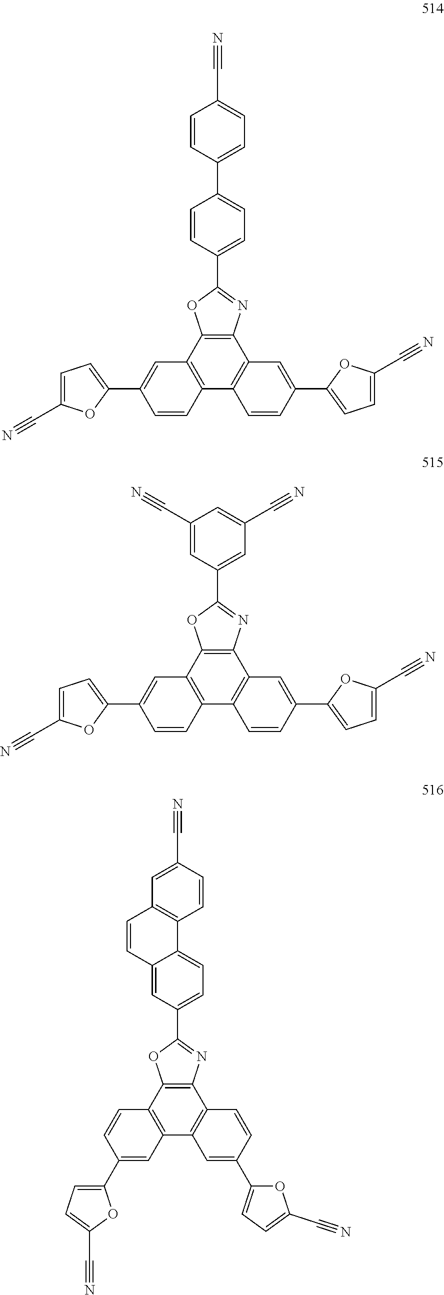 [SMILES: N#Cc1ccc(-c2ccc(-c3nc4c5cc(-c6ccc(C#N)o6)ccc5c5ccc(-c6ccc(C#N)o6)cc5c4o3)cc2)cc1 ; N#Cc1cc(C#N)cc(-c2nc3c4cc(-c5ccc(C#N)o5)ccc4c4ccc(-c5ccc(C#N)o5)cc4c3o2)c1 ; N#Cc1ccc2c(ccc3cc(-c4nc5c6ccc(-c7ccc(C#N)o7)cc6c6cc(-c7ccc(C#N)o7)ccc6c5o4)ccc32)c1]

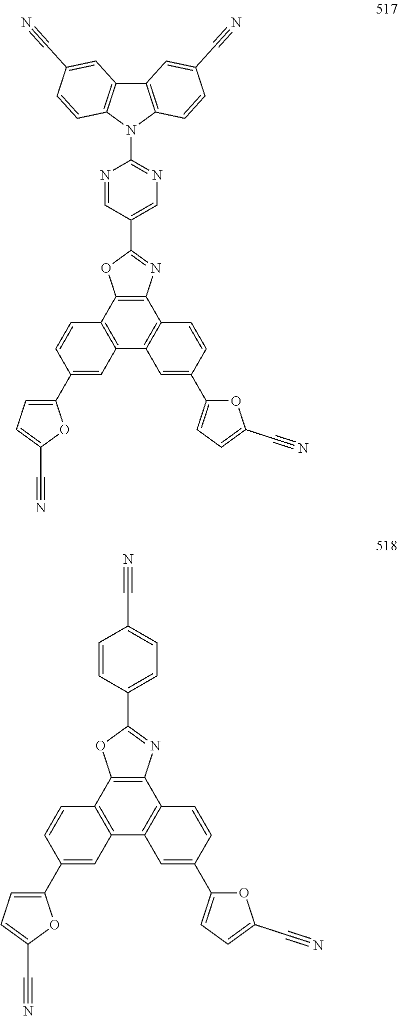 [SMILES: N#Cc1ccc2c(c1)c1cc(C#N)ccc1n2-c1ncc(-c2nc3c4ccc(-c5ccc(C#N)o5)cc4c4cc(-c5ccc(C#N)o5)ccc4c3o2)cn1 ; N#Cc1ccc(-c2nc3c4ccc(-c5ccc(C#N)o5)cc4c4cc(-c5ccc(C#N)o5)ccc4c3o2)cc1]

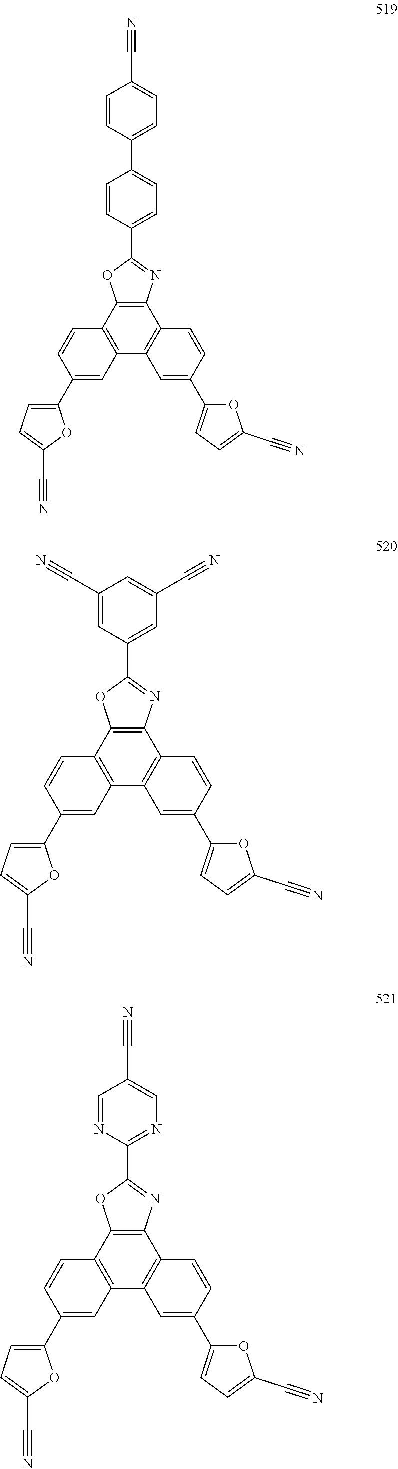 [SMILES: N#Cc1ccc(-c2ccc(-c3nc4c5ccc(-c6ccc(C#N)o6)cc5c5cc(-c6ccc(C#N)o6)ccc5c4o3)cc2)cc1 ; N#Cc1cc(C#N)cc(-c2nc3c4ccc(-c5ccc(C#N)o5)cc4c4cc(-c5ccc(C#N)o5)ccc4c3o2)c1 ; N#Cc1cnc(-c2nc3c4ccc(-c5ccc(C#N)o5)cc4c4cc(-c5ccc(C#N)o5)ccc4c3o2)nc1]

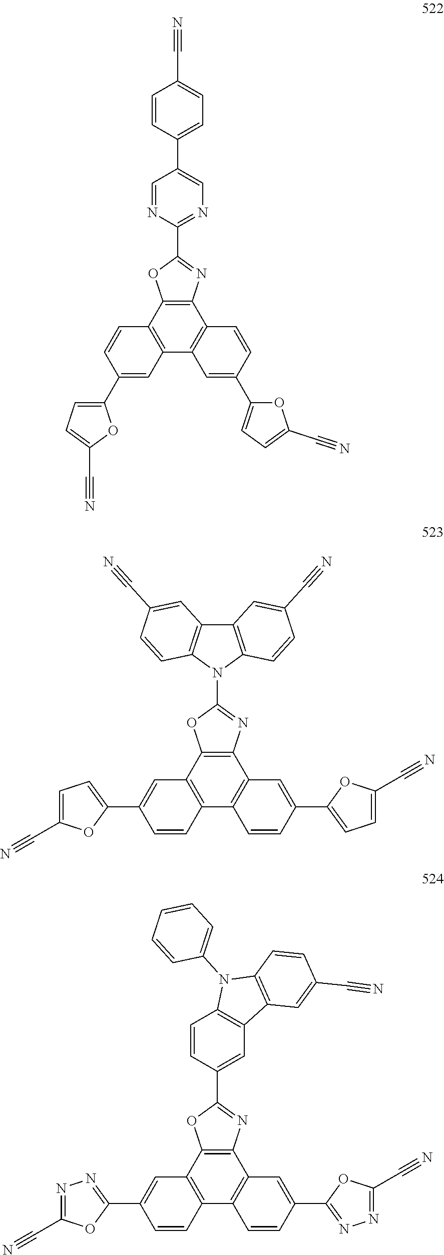 [SMILES: N#Cc1ccc(-c2cnc(-c3nc4c5ccc(-c6ccc(C#N)o6)cc5c5cc(-c6ccc(C#N)o6)ccc5c4o3)nc2)cc1 ; N#Cc1ccc2c(c1)c1cc(C#N)ccc1n2-c1nc2c3cc(-c4ccc(C#N)o4)ccc3c3ccc(-c4ccc(C#N)o4)cc3c2o1 ; N#Cc1ccc2c(c1)c1cc(-c3nc4c5cc(-c6nnc(C#N)o6)ccc5c5ccc(-c6nnc(C#N)o6)cc5c4o3)ccc1n2-c1ccccc1]

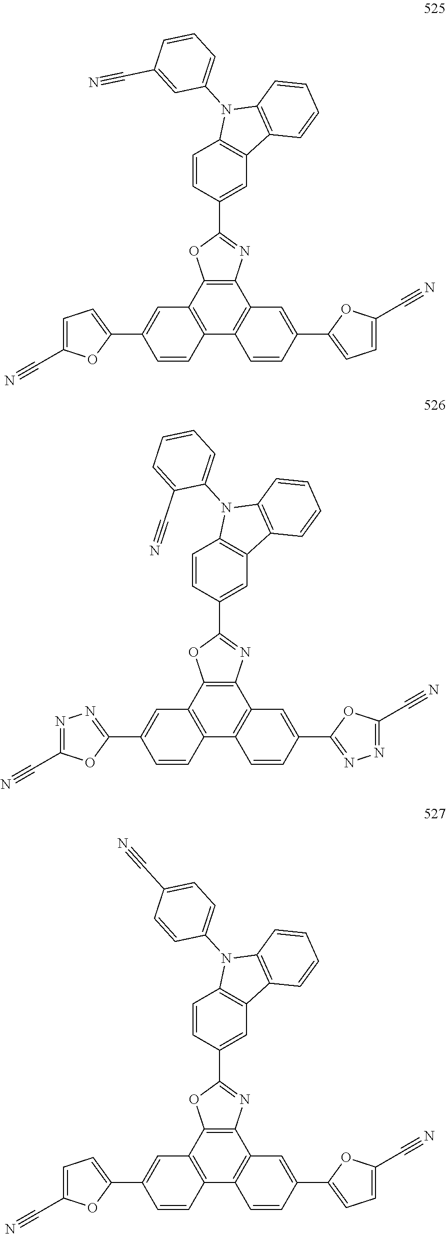 [SMILES: N#Cc1cccc(-n2c3ccccc3c3cc(-c4nc5c6cc(-c7ccc(C#N)o7)ccc6c6ccc(-c7ccc(C#N)o7)cc6c5o4)ccc32)c1 ; N#Cc1nnc(-c2ccc3c4ccc(-c5nnc(C#N)o5)cc4c4oc(-c5ccc6c(c5)c5ccccc5n6-c5ccccc5C#N)nc4c3c2)o1 ; N#Cc1ccc(-n2c3ccccc3c3cc(-c4nc5c6cc(-c7ccc(C#N)o7)ccc6c6ccc(-c7ccc(C#N)o7)cc6c5o4)ccc32)cc1]

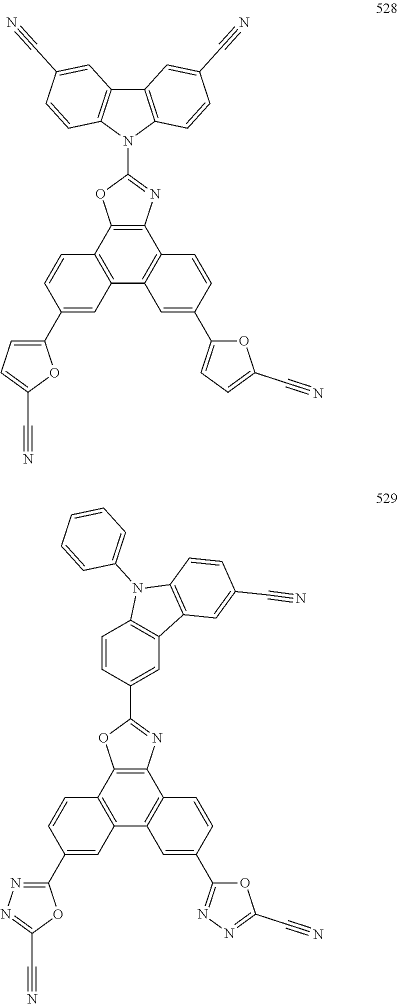 [SMILES: N#Cc1ccc2c(c1)c1cc(C#N)ccc1n2-c1nc2c3ccc(-c4ccc(C#N)o4)cc3c3cc(-c4ccc(C#N)o4)ccc3c2o1 ; N#Cc1ccc2c(c1)c1cc(-c3nc4c5ccc(-c6nnc(C#N)o6)cc5c5cc(-c6nnc(C#N)o6)ccc5c4o3)ccc1n2-c1ccccc1]

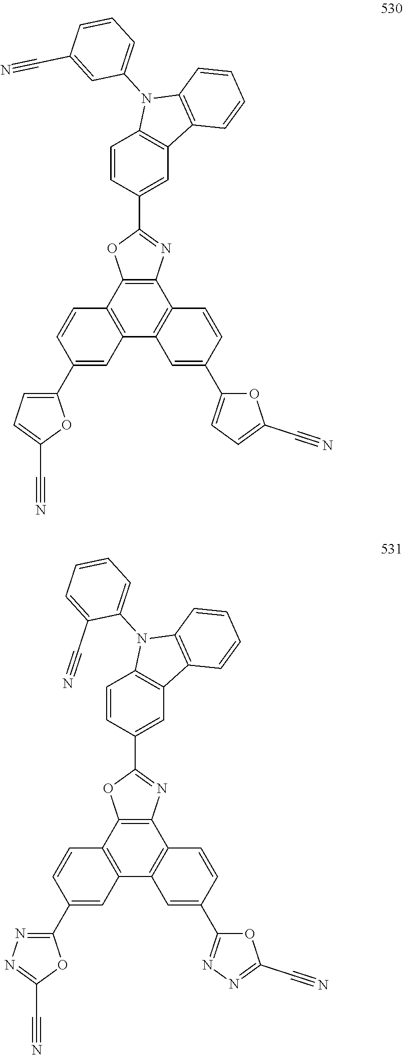 [SMILES: N#Cc1cccc(-n2c3ccccc3c3cc(-c4nc5c6ccc(-c7ccc(C#N)o7)cc6c6cc(-c7ccc(C#N)o7)ccc6c5o4)ccc32)c1 ; N#Cc1nnc(-c2ccc3c(c2)c2cc(-c4nnc(C#N)o4)ccc2c2oc(-c4ccc5c(c4)c4ccccc4n5-c4ccccc4C#N)nc32)o1]

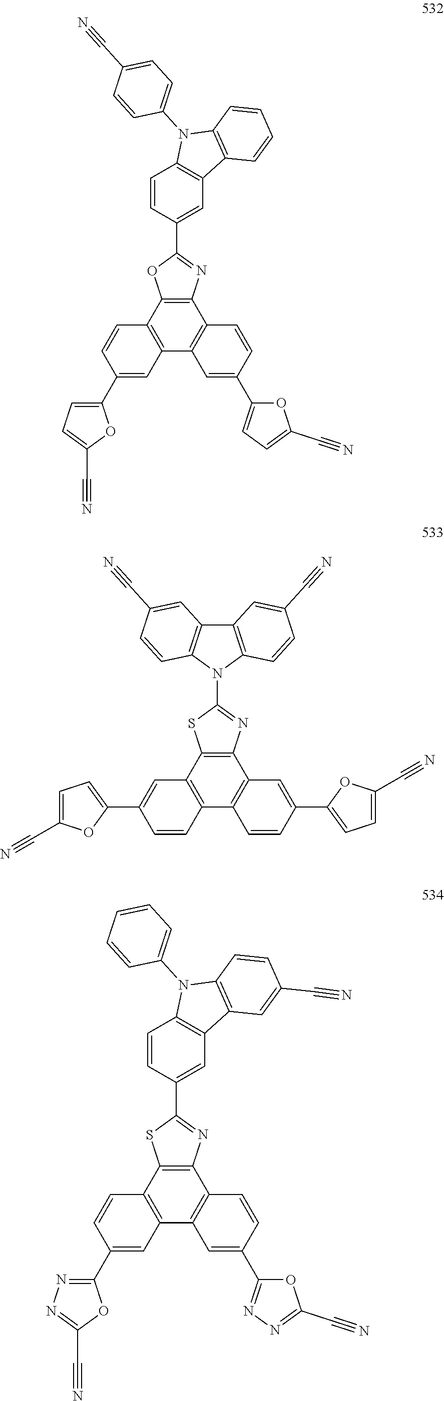 [SMILES: N#Cc1ccc(-n2c3ccccc3c3cc(-c4nc5c6ccc(-c7ccc(C#N)o7)cc6c6cc(-c7ccc(C#N)o7)ccc6c5o4)ccc32)cc1 ; N#Cc1ccc2c(c1)c1cc(C#N)ccc1n2-c1nc2c3cc(-c4ccc(C#N)o4)ccc3c3ccc(-c4ccc(C#N)o4)cc3c2s1 ; N#Cc1ccc2c(c1)c1cc(-c3nc4c5ccc(-c6nnc(C#N)o6)cc5c5cc(-c6nnc(C#N)o6)ccc5c4s3)ccc1n2-c1ccccc1]

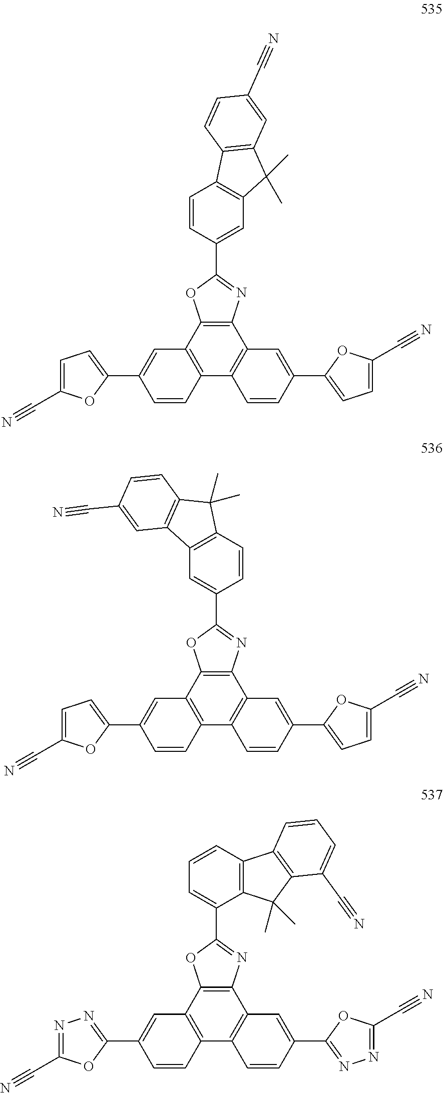 [SMILES: CC1(C)c2cc(C#N)ccc2-c2ccc(-c3nc4c5cc(-c6ccc(C#N)o6)ccc5c5ccc(-c6ccc(C#N)o6)cc5c4o3)cc21 ; CC1(C)c2ccc(C#N)cc2-c2cc(-c3nc4c5cc(-c6ccc(C#N)o6)ccc5c5ccc(-c6ccc(C#N)o6)cc5c4o3)ccc21 ; CC1(C)c2c(C#N)cccc2-c2cccc(-c3nc4c5cc(-c6nnc(C#N)o6)ccc5c5ccc(-c6nnc(C#N)o6)cc5c4o3)c21]

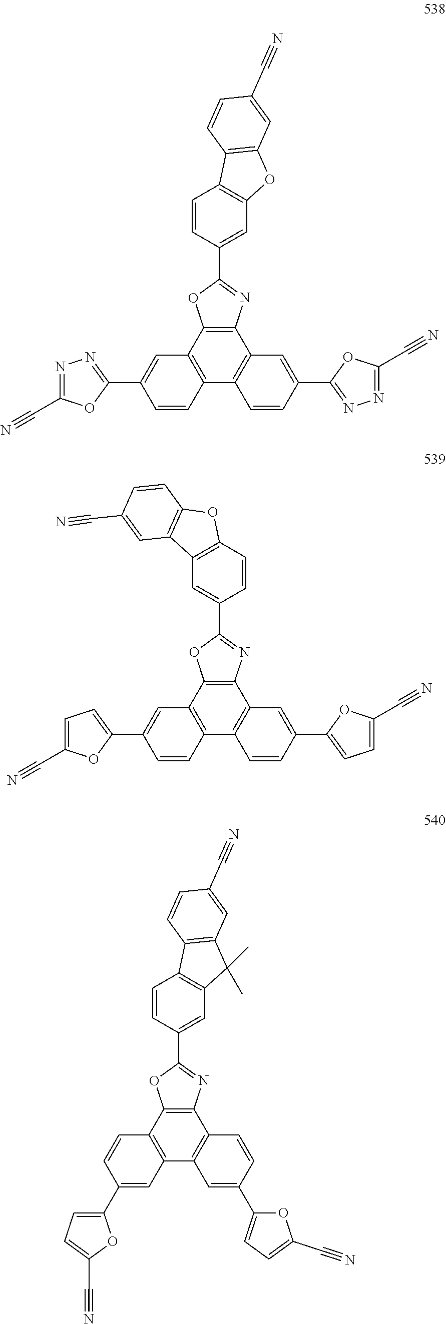 [SMILES: N#Cc1ccc2c(c1)oc1cc(-c3nc4c5cc(-c6nnc(C#N)o6)ccc5c5ccc(-c6nnc(C#N)o6)cc5c4o3)ccc12 ; N#Cc1ccc2oc3ccc(-c4nc5c6cc(-c7ccc(C#N)o7)ccc6c6ccc(-c7ccc(C#N)o7)cc6c5o4)cc3c2c1 ; CC1(C)c2cc(C#N)ccc2-c2ccc(-c3nc4c5ccc(-c6ccc(C#N)o6)cc5c5cc(-c6ccc(C#N)o6)ccc5c4o3)cc21]

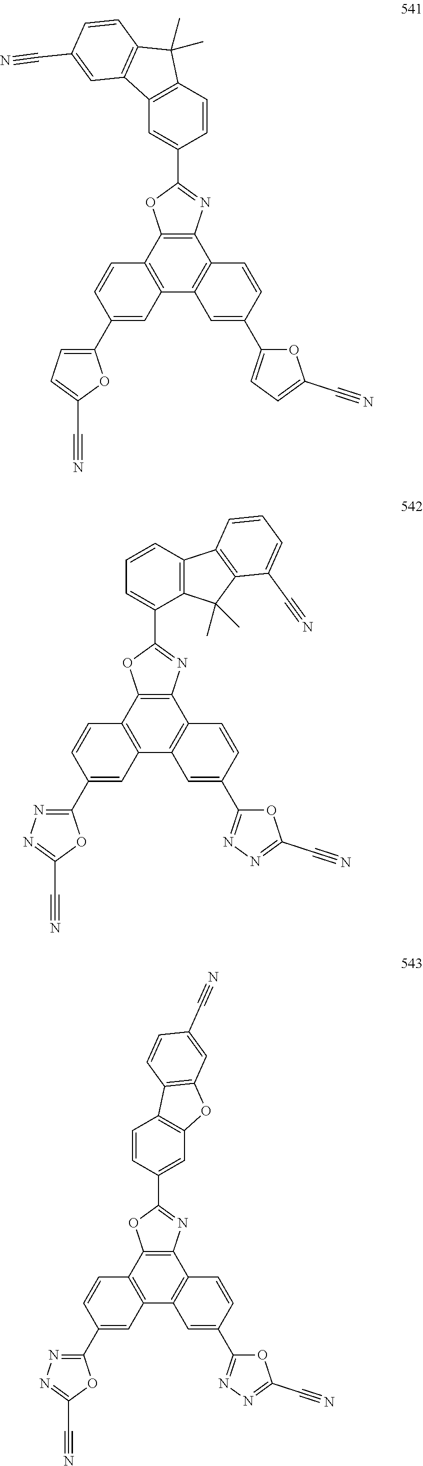 [SMILES: CC1(C)c2ccc(C#N)cc2-c2cc(-c3nc4c5ccc(-c6ccc(C#N)o6)cc5c5cc(-c6ccc(C#N)o6)ccc5c4o3)ccc21 ; CC1(C)c2c(C#N)cccc2-c2cccc(-c3nc4c5ccc(-c6nnc(C#N)o6)cc5c5cc(-c6nnc(C#N)o6)ccc5c4o3)c21 ; N#Cc1ccc2c(c1)oc1cc(-c3nc4c5ccc(-c6nnc(C#N)o6)cc5c5cc(-c6nnc(C#N)o6)ccc5c4o3)ccc12]

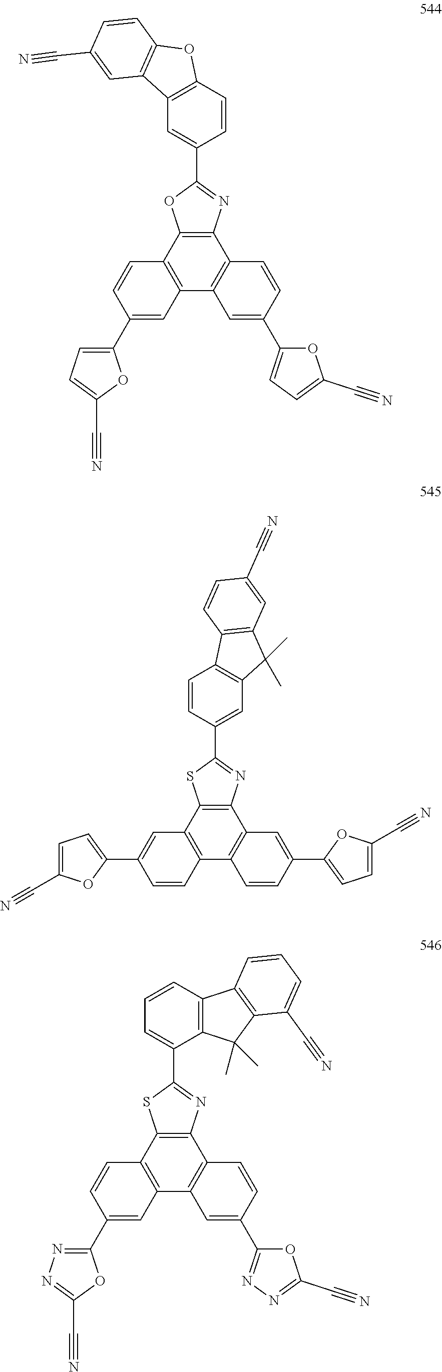 [SMILES: N#Cc1ccc2oc3ccc(-c4nc5c6ccc(-c7ccc(C#N)o7)cc6c6cc(-c7ccc(C#N)o7)ccc6c5o4)cc3c2c1 ; CC1(C)c2cc(C#N)ccc2-c2ccc(-c3nc4c5cc(-c6ccc(C#N)o6)ccc5c5ccc(-c6ccc(C#N)o6)cc5c4s3)cc21 ; CC1(C)c2c(C#N)cccc2-c2cccc(-c3nc4c5ccc(-c6nnc(C#N)o6)cc5c5cc(-c6nnc(C#N)o6)ccc5c4s3)c21]

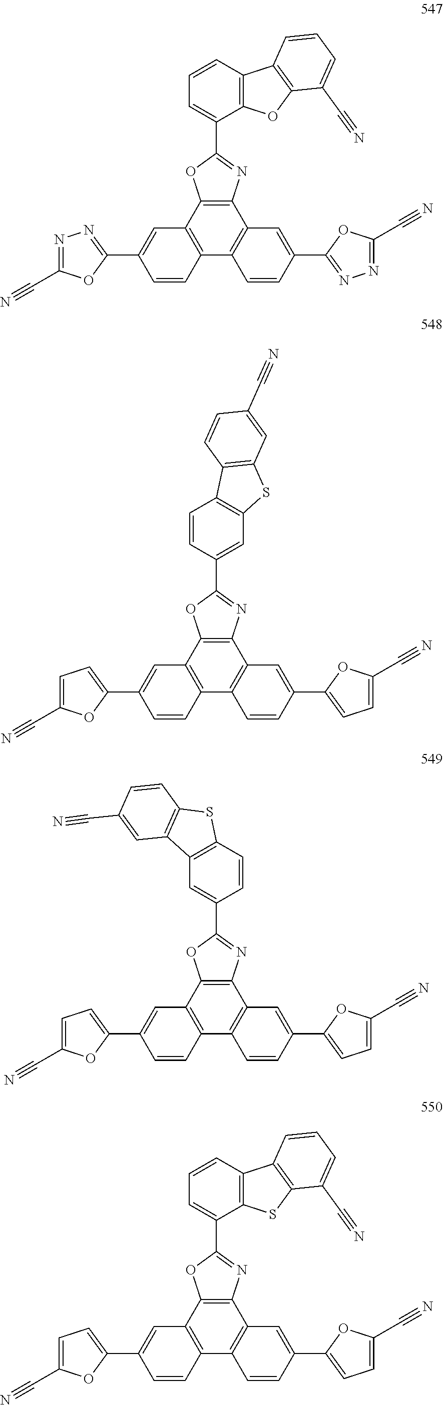 [SMILES: N#Cc1nnc(-c2ccc3c4ccc(-c5nnc(C#N)o5)cc4c4oc(-c5cccc6c5oc5c(C#N)cccc56)nc4c3c2)o1 ; N#Cc1ccc2c(c1)sc1cc(-c3nc4c5cc(-c6ccc(C#N)o6)ccc5c5ccc(-c6ccc(C#N)o6)cc5c4o3)ccc12 ; N#Cc1ccc2sc3ccc(-c4nc5c6cc(-c7ccc(C#N)o7)ccc6c6ccc(-c7ccc(C#N)o7)cc6c5o4)cc3c2c1 ; N#Cc1ccc(-c2ccc3c4ccc(-c5ccc(C#N)o5)cc4c4oc(-c5cccc6c5sc5c(C#N)cccc56)nc4c3c2)o1]

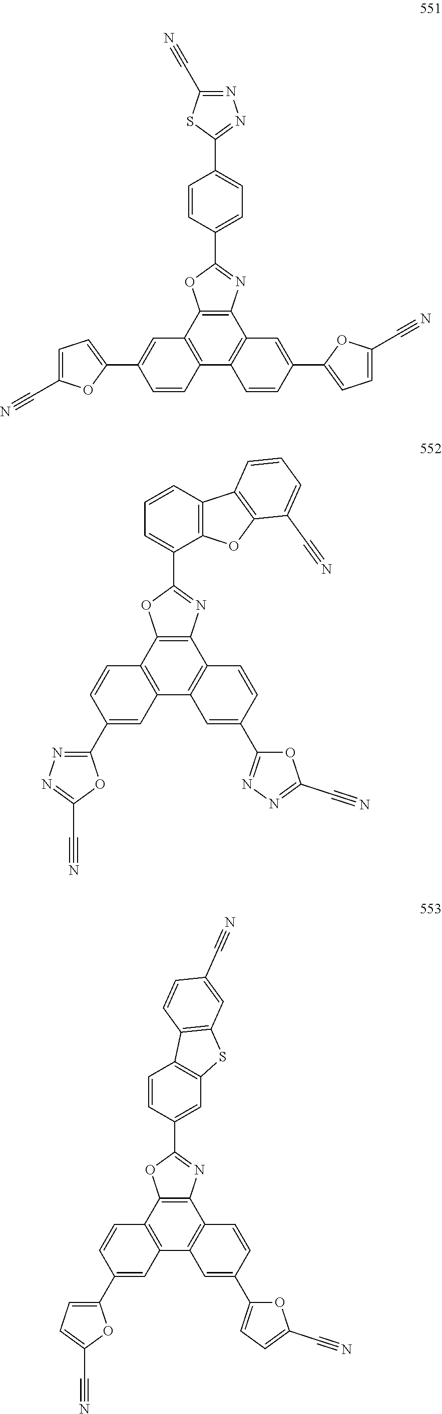 [SMILES: N#Cc1ccc(-c2ccc3c4ccc(-c5ccc(C#N)o5)cc4c4oc(-c5ccc(-c6nnc(C#N)s6)cc5)nc4c3c2)o1 ; N#Cc1nnc(-c2ccc3c(c2)c2cc(-c4nnc(C#N)o4)ccc2c2oc(-c4cccc5c4oc4c(C#N)cccc45)nc32)o1 ; N#Cc1ccc2c(c1)sc1cc(-c3nc4c5ccc(-c6ccc(C#N)o6)cc5c5cc(-c6ccc(C#N)o6)ccc5c4o3)ccc12]

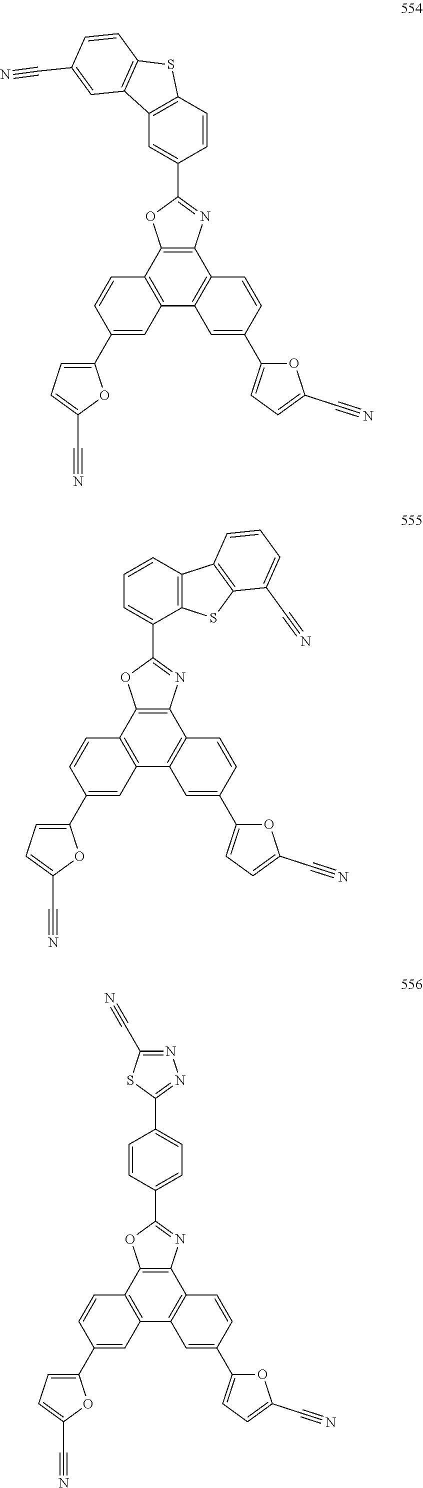 [SMILES: N#Cc1ccc2sc3ccc(-c4nc5c6ccc(-c7ccc(C#N)o7)cc6c6cc(-c7ccc(C#N)o7)ccc6c5o4)cc3c2c1 ; N#Cc1ccc(-c2ccc3c(c2)c2cc(-c4ccc(C#N)o4)ccc2c2oc(-c4cccc5c4sc4c(C#N)cccc45)nc32)o1 ; N#Cc1ccc(-c2ccc3c(c2)c2cc(-c4ccc(C#N)o4)ccc2c2oc(-c4ccc(-c5nnc(C#N)s5)cc4)nc32)o1]

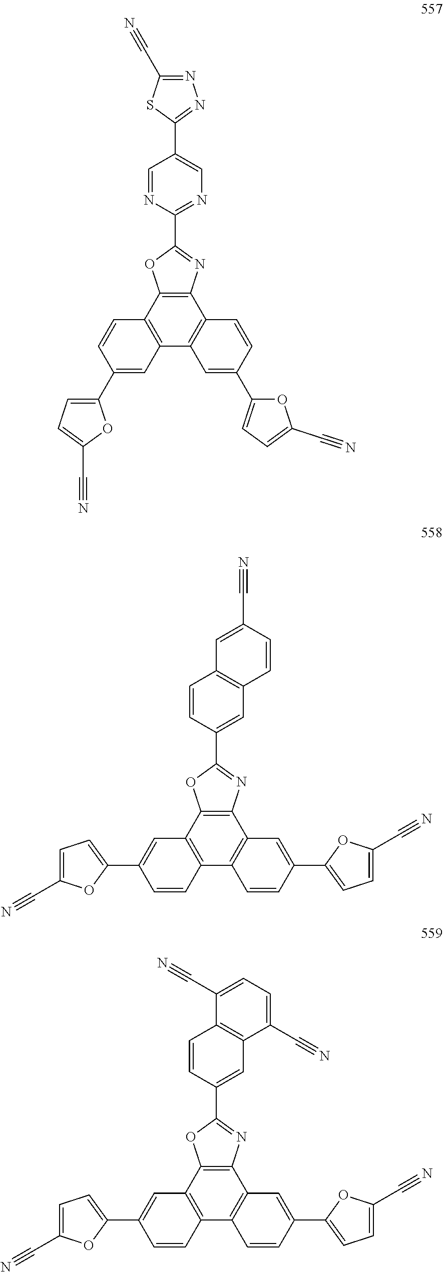 [SMILES: N#Cc1ccc(-c2ccc3c(c2)c2cc(-c4ccc(C#N)o4)ccc2c2oc(-c4ncc(-c5nnc(C#N)s5)cn4)nc32)o1 ; N#Cc1ccc2cc(-c3nc4c5cc(-c6ccc(C#N)o6)ccc5c5ccc(-c6ccc(C#N)o6)cc5c4o3)ccc2c1 ; N#Cc1ccc(-c2ccc3c4ccc(-c5ccc(C#N)o5)cc4c4oc(-c5ccc6c(C#N)ccc(C#N)c6c5)nc4c3c2)o1]

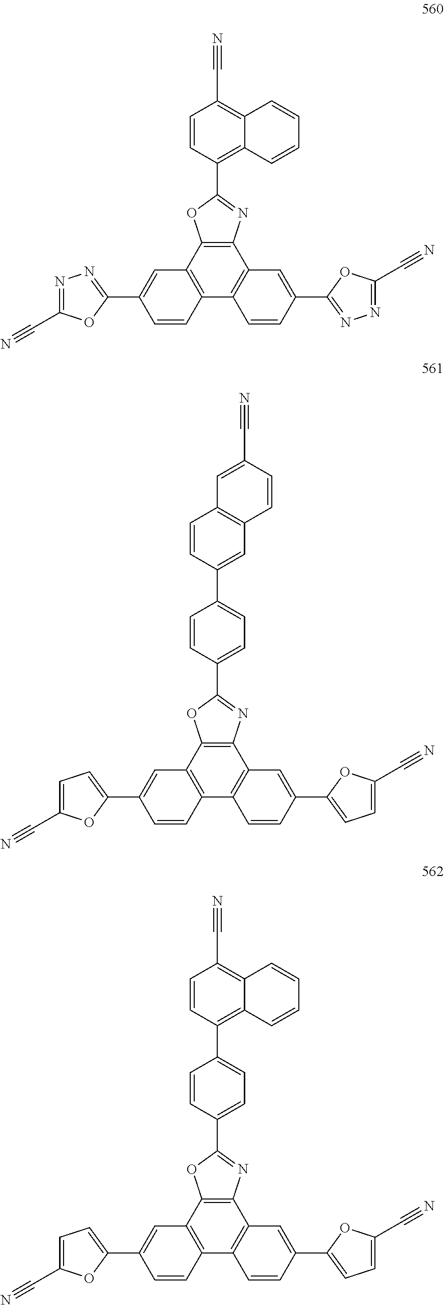 [SMILES: N#Cc1nnc(-c2ccc3c4ccc(-c5nnc(C#N)o5)cc4c4oc(-c5ccc(C#N)c6ccccc56)nc4c3c2)o1 ; N#Cc1ccc2cc(-c3ccc(-c4nc5c6cc(-c7ccc(C#N)o7)ccc6c6ccc(-c7ccc(C#N)o7)cc6c5o4)cc3)ccc2c1 ; N#Cc1ccc(-c2ccc3c4ccc(-c5ccc(C#N)o5)cc4c4oc(-c5ccc(-c6ccc(C#N)c7ccccc67)cc5)nc4c3c2)o1]

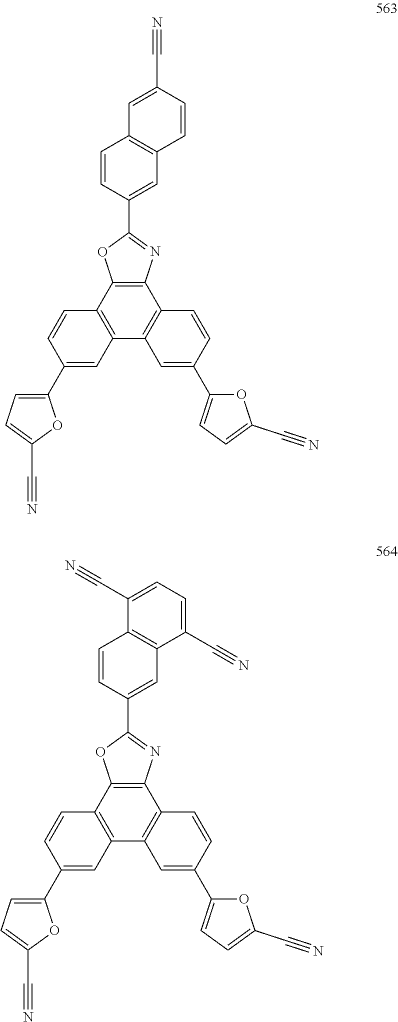 [SMILES: N#Cc1ccc2cc(-c3nc4c5ccc(-c6ccc(C#N)o6)cc5c5cc(-c6ccc(C#N)o6)ccc5c4o3)ccc2c1 ; N#Cc1ccc(-c2ccc3c(c2)c2cc(-c4ccc(C#N)o4)ccc2c2oc(-c4ccc5c(C#N)ccc(C#N)c5c4)nc32)o1]

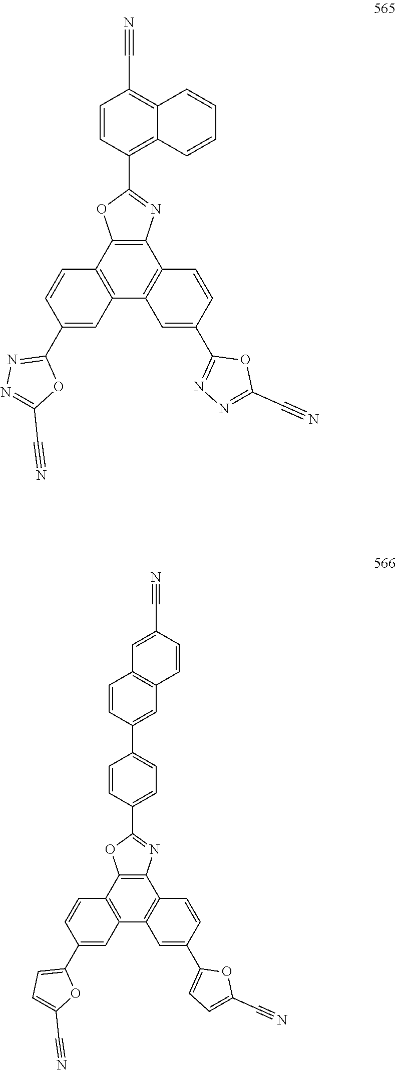 [SMILES: N#Cc1nnc(-c2ccc3c(c2)c2cc(-c4nnc(C#N)o4)ccc2c2oc(-c4ccc(C#N)c5ccccc45)nc32)o1 ; N#Cc1ccc2cc(-c3ccc(-c4nc5c6ccc(-c7ccc(C#N)o7)cc6c6cc(-c7ccc(C#N)o7)ccc6c5o4)cc3)ccc2c1]

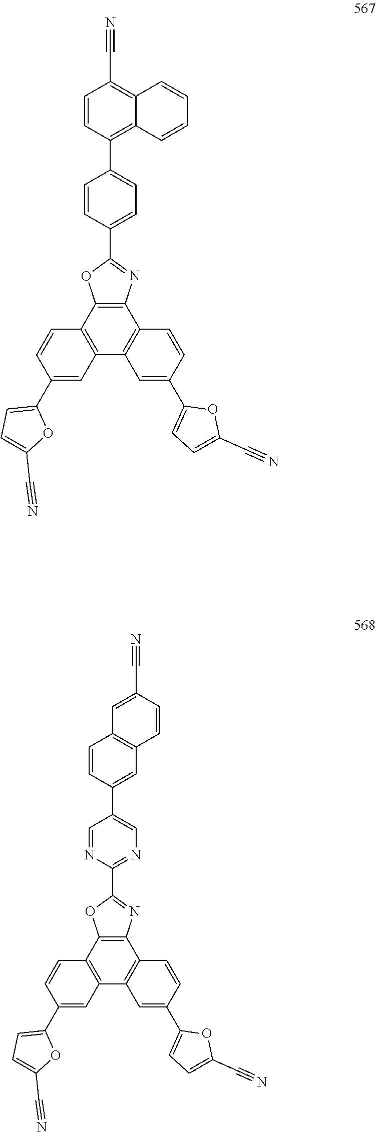 [SMILES: N#Cc1ccc(-c2ccc3c(c2)c2cc(-c4ccc(C#N)o4)ccc2c2oc(-c4ccc(-c5ccc(C#N)c6ccccc56)cc4)nc32)o1 ; N#Cc1ccc2cc(-c3cnc(-c4nc5c6ccc(-c7ccc(C#N)o7)cc6c6cc(-c7ccc(C#N)o7)ccc6c5o4)nc3)ccc2c1]

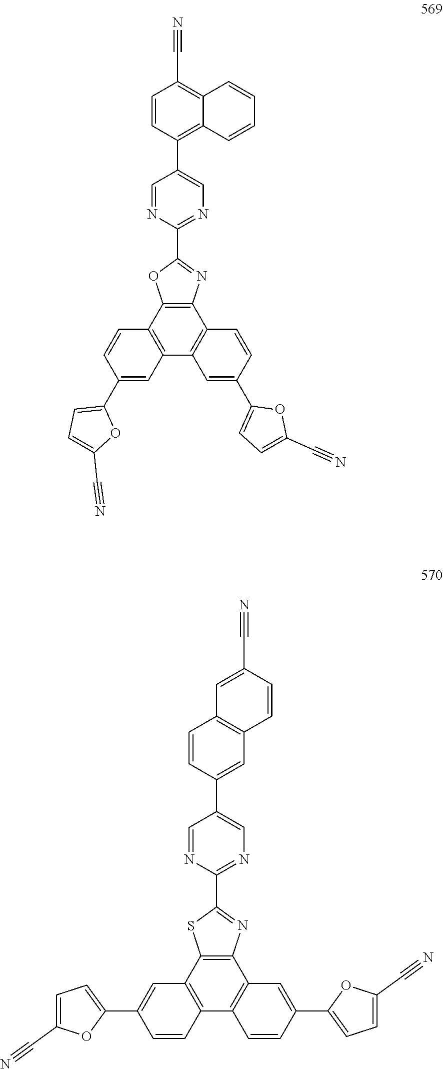 [SMILES: N#Cc1ccc(-c2ccc3c(c2)c2cc(-c4ccc(C#N)o4)ccc2c2oc(-c4ncc(-c5ccc(C#N)c6ccccc56)cn4)nc32)o1 ; N#Cc1ccc2cc(-c3cnc(-c4nc5c6cc(-c7ccc(C#N)o7)ccc6c6ccc(-c7ccc(C#N)o7)cc6c5s4)nc3)ccc2c1]

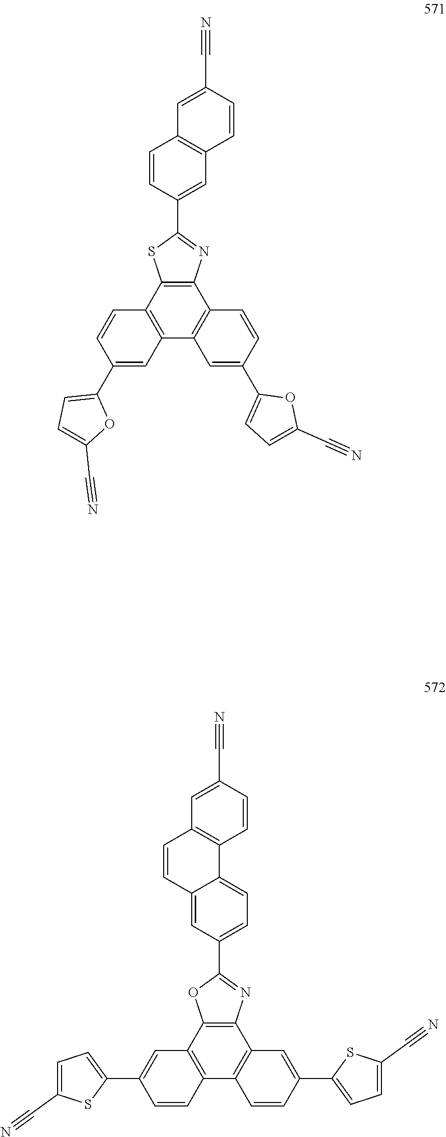 [SMILES: N#Cc1ccc2cc(-c3nc4c5ccc(-c6ccc(C#N)o6)cc5c5cc(-c6ccc(C#N)o6)ccc5c4s3)ccc2c1 ; N#Cc1ccc2c(ccc3cc(-c4nc5c6cc(-c7ccc(C#N)s7)ccc6c6ccc(-c7ccc(C#N)s7)cc6c5o4)ccc32)c1]

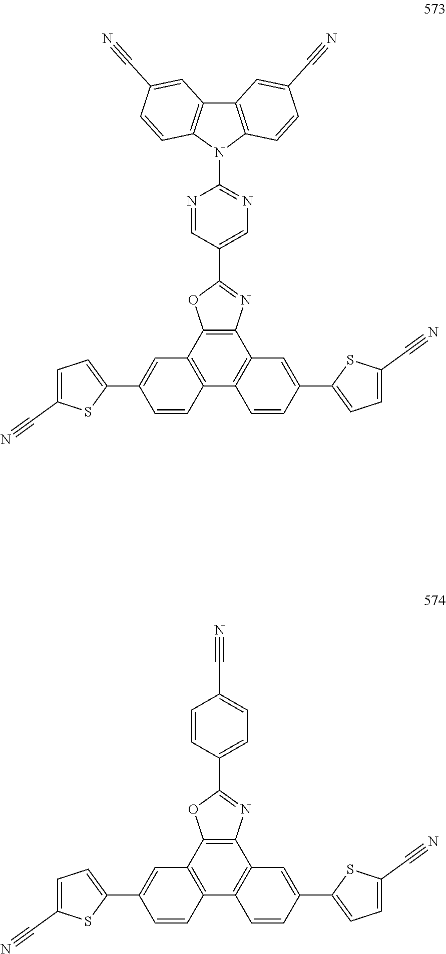 [SMILES: N#Cc1ccc2c(c1)c1cc(C#N)ccc1n2-c1ncc(-c2nc3c4cc(-c5ccc(C#N)s5)ccc4c4ccc(-c5ccc(C#N)s5)cc4c3o2)cn1 ; N#Cc1ccc(-c2nc3c4cc(-c5ccc(C#N)s5)ccc4c4ccc(-c5ccc(C#N)s5)cc4c3o2)cc1]

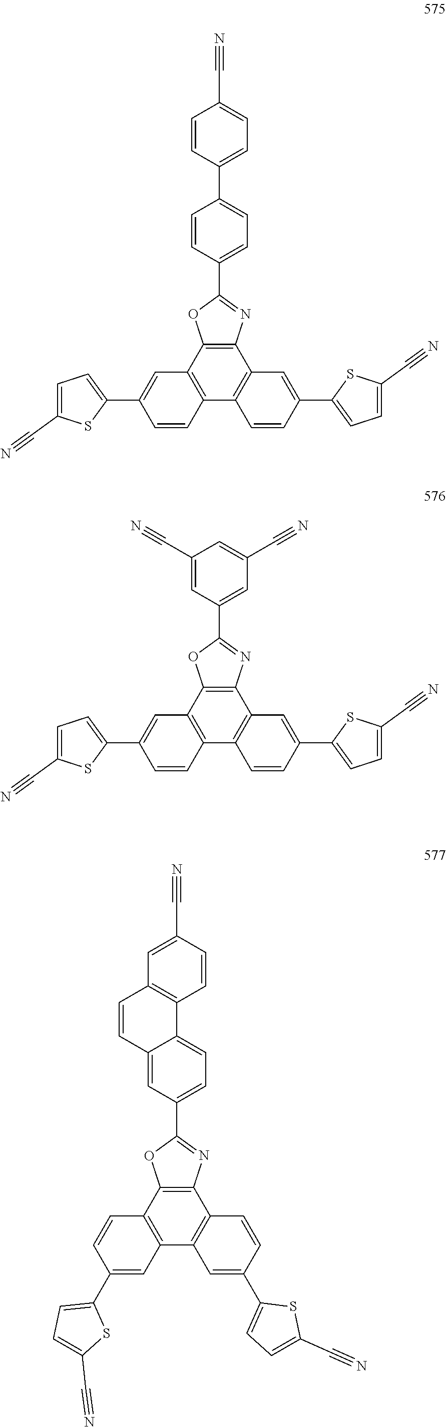 [SMILES: N#Cc1ccc(-c2ccc(-c3nc4c5cc(-c6ccc(C#N)s6)ccc5c5ccc(-c6ccc(C#N)s6)cc5c4o3)cc2)cc1 ; N#Cc1cc(C#N)cc(-c2nc3c4cc(-c5ccc(C#N)s5)ccc4c4ccc(-c5ccc(C#N)s5)cc4c3o2)c1 ; N#Cc1ccc2c(ccc3cc(-c4nc5c6ccc(-c7ccc(C#N)s7)cc6c6cc(-c7ccc(C#N)s7)ccc6c5o4)ccc32)c1]

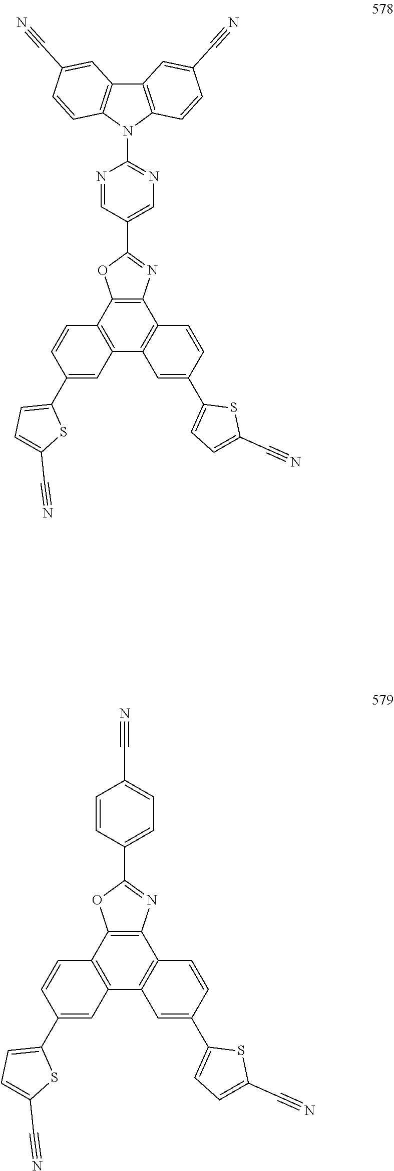 [SMILES: N#Cc1ccc2c(c1)c1cc(C#N)ccc1n2-c1ncc(-c2nc3c4ccc(-c5ccc(C#N)s5)cc4c4cc(-c5ccc(C#N)s5)ccc4c3o2)cn1 ; N#Cc1ccc(-c2nc3c4ccc(-c5ccc(C#N)s5)cc4c4cc(-c5ccc(C#N)s5)ccc4c3o2)cc1]

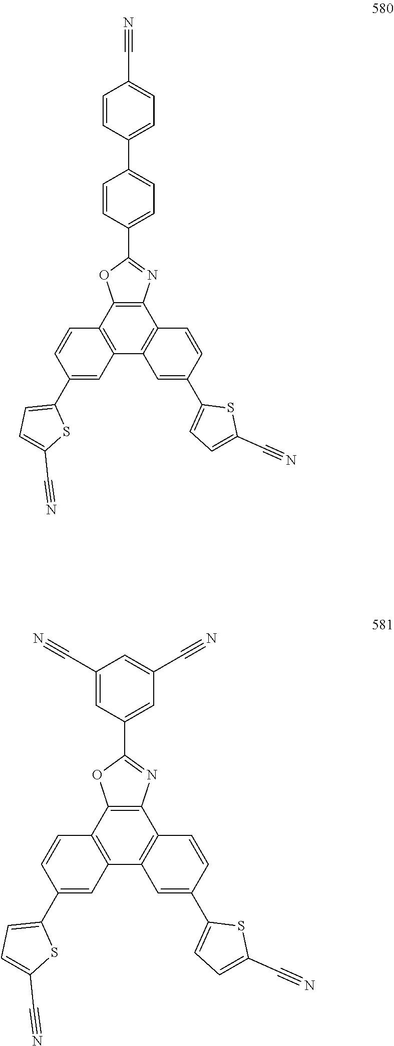 [SMILES: N#Cc1ccc(-c2ccc(-c3nc4c5ccc(-c6ccc(C#N)s6)cc5c5cc(-c6ccc(C#N)s6)ccc5c4o3)cc2)cc1 ; N#Cc1cc(C#N)cc(-c2nc3c4ccc(-c5ccc(C#N)s5)cc4c4cc(-c5ccc(C#N)s5)ccc4c3o2)c1]

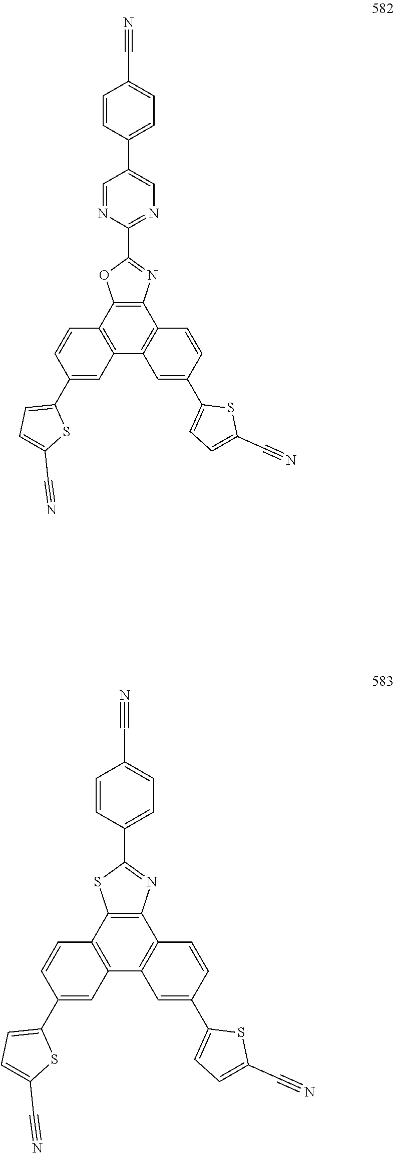 [SMILES: N#Cc1ccc(-c2cnc(-c3nc4c5ccc(-c6ccc(C#N)s6)cc5c5cc(-c6ccc(C#N)s6)ccc5c4o3)nc2)cc1 ; N#Cc1ccc(-c2nc3c4ccc(-c5ccc(C#N)s5)cc4c4cc(-c5ccc(C#N)s5)ccc4c3s2)cc1]

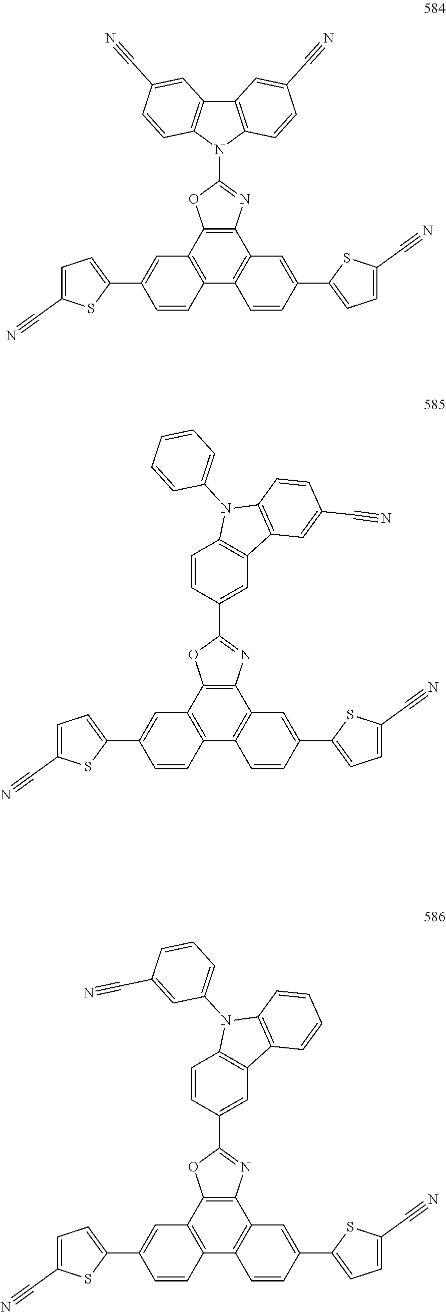 [SMILES: N#Cc1ccc2c(c1)c1cc(C#N)ccc1n2-c1nc2c3cc(-c4ccc(C#N)s4)ccc3c3ccc(-c4ccc(C#N)s4)cc3c2o1 ; N#Cc1ccc2c(c1)c1cc(-c3nc4c5cc(-c6ccc(C#N)s6)ccc5c5ccc(-c6ccc(C#N)s6)cc5c4o3)ccc1n2-c1ccccc1 ; N#Cc1cccc(-n2c3ccccc3c3cc(-c4nc5c6cc(-c7ccc(C#N)s7)ccc6c6ccc(-c7ccc(C#N)s7)cc6c5o4)ccc32)c1]

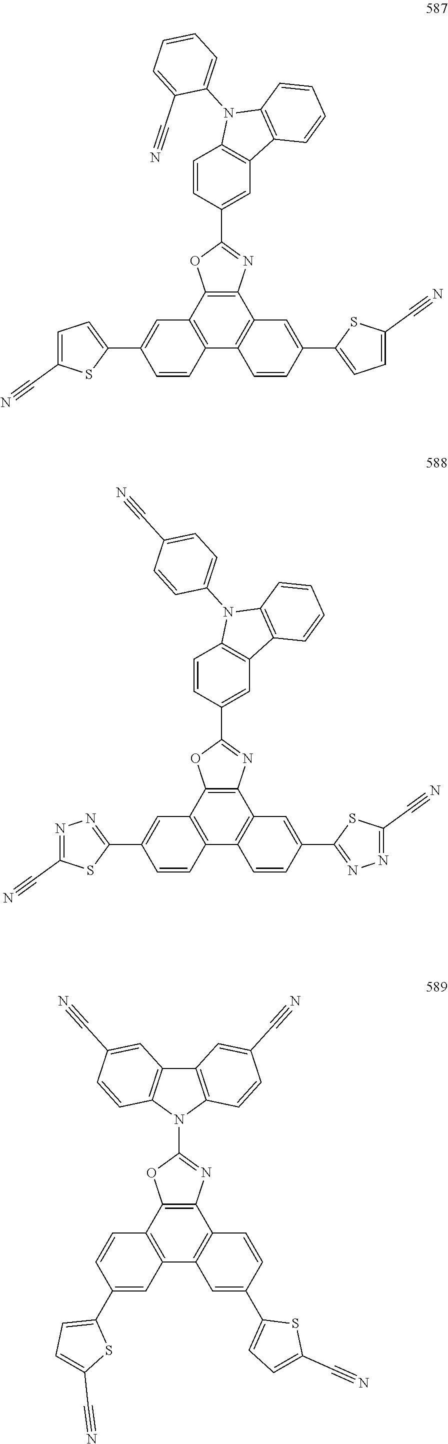 [SMILES: N#Cc1ccc(-c2ccc3c4ccc(-c5ccc(C#N)s5)cc4c4oc(-c5ccc6c(c5)c5ccccc5n6-c5ccccc5C#N)nc4c3c2)s1 ; N#Cc1ccc(-n2c3ccccc3c3cc(-c4nc5c6cc(-c7nnc(C#N)s7)ccc6c6ccc(-c7nnc(C#N)s7)cc6c5o4)ccc32)cc1 ; N#Cc1ccc2c(c1)c1cc(C#N)ccc1n2-c1nc2c3ccc(-c4ccc(C#N)s4)cc3c3cc(-c4ccc(C#N)s4)ccc3c2o1]

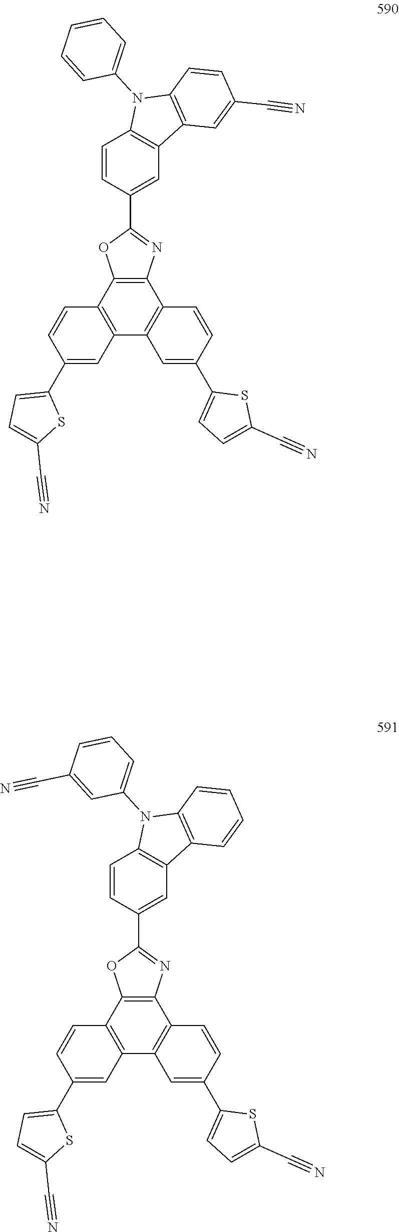 [SMILES: N#Cc1ccc2c(c1)c1cc(-c3nc4c5ccc(-c6ccc(C#N)s6)cc5c5cc(-c6ccc(C#N)s6)ccc5c4o3)ccc1n2-c1ccccc1 ; N#Cc1cccc(-n2c3ccccc3c3cc(-c4nc5c6ccc(-c7ccc(C#N)s7)cc6c6cc(-c7ccc(C#N)s7)ccc6c5o4)ccc32)c1]

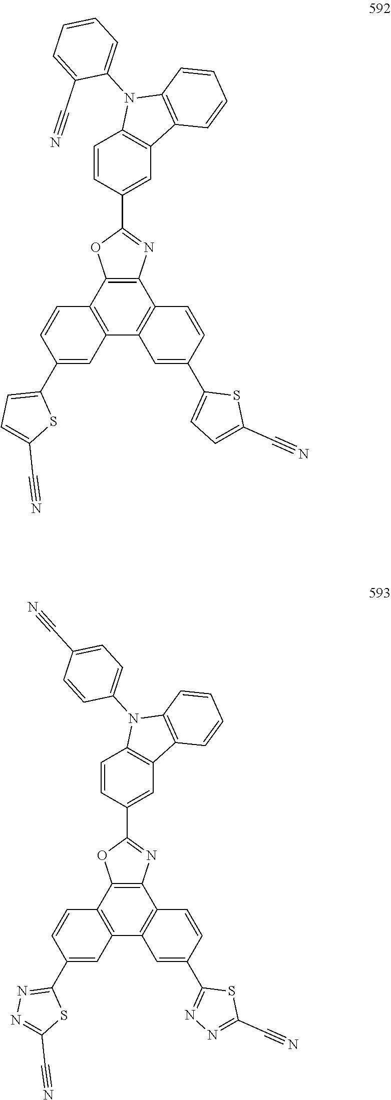 [SMILES: N#Cc1ccc(-c2ccc3c(c2)c2cc(-c4ccc(C#N)s4)ccc2c2oc(-c4ccc5c(c4)c4ccccc4n5-c4ccccc4C#N)nc32)s1 ; N#Cc1ccc(-n2c3ccccc3c3cc(-c4nc5c6ccc(-c7nnc(C#N)s7)cc6c6cc(-c7nnc(C#N)s7)ccc6c5o4)ccc32)cc1]

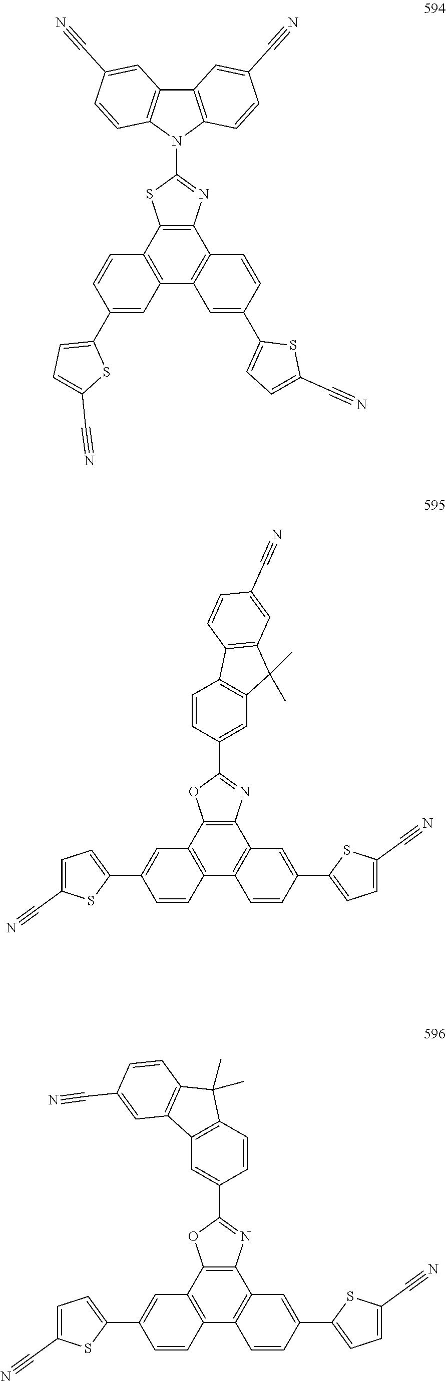 [SMILES: N#Cc1ccc2c(c1)c1cc(C#N)ccc1n2-c1nc2c3ccc(-c4ccc(C#N)s4)cc3c3cc(-c4ccc(C#N)s4)ccc3c2s1 ; CC1(C)c2cc(C#N)ccc2-c2ccc(-c3nc4c5cc(-c6ccc(C#N)s6)ccc5c5ccc(-c6ccc(C#N)s6)cc5c4o3)cc21 ; CC1(C)c2ccc(C#N)cc2-c2cc(-c3nc4c5cc(-c6ccc(C#N)s6)ccc5c5ccc(-c6ccc(C#N)s6)cc5c4o3)ccc21]

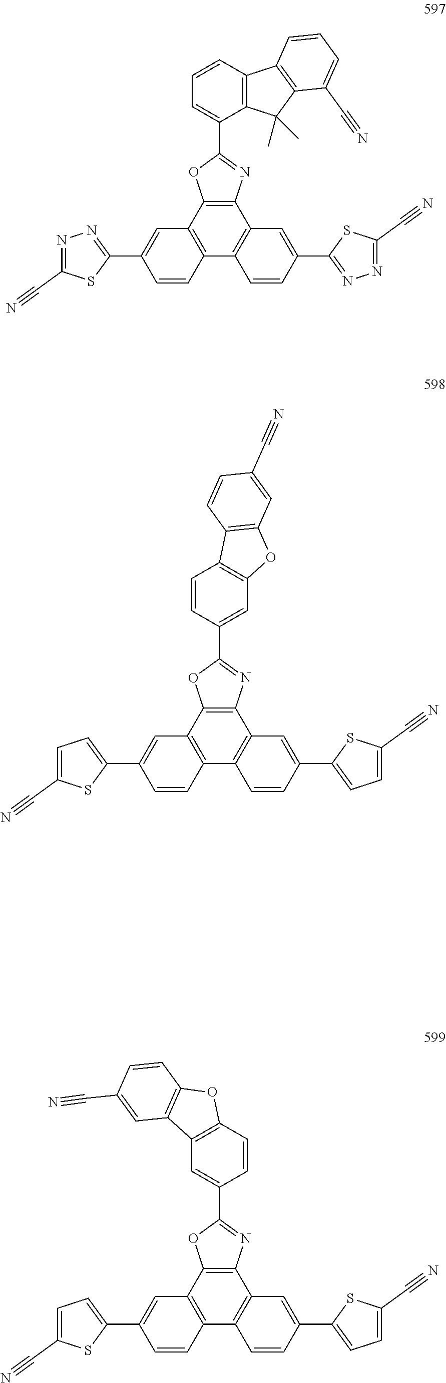 [SMILES: CC1(C)c2c(C#N)cccc2-c2cccc(-c3nc4c5cc(-c6nnc(C#N)s6)ccc5c5ccc(-c6nnc(C#N)s6)cc5c4o3)c21 ; N#Cc1ccc2c(c1)oc1cc(-c3nc4c5cc(-c6ccc(C#N)s6)ccc5c5ccc(-c6ccc(C#N)s6)cc5c4o3)ccc12 ; N#Cc1ccc2oc3ccc(-c4nc5c6cc(-c7ccc(C#N)s7)ccc6c6ccc(-c7ccc(C#N)s7)cc6c5o4)cc3c2c1]

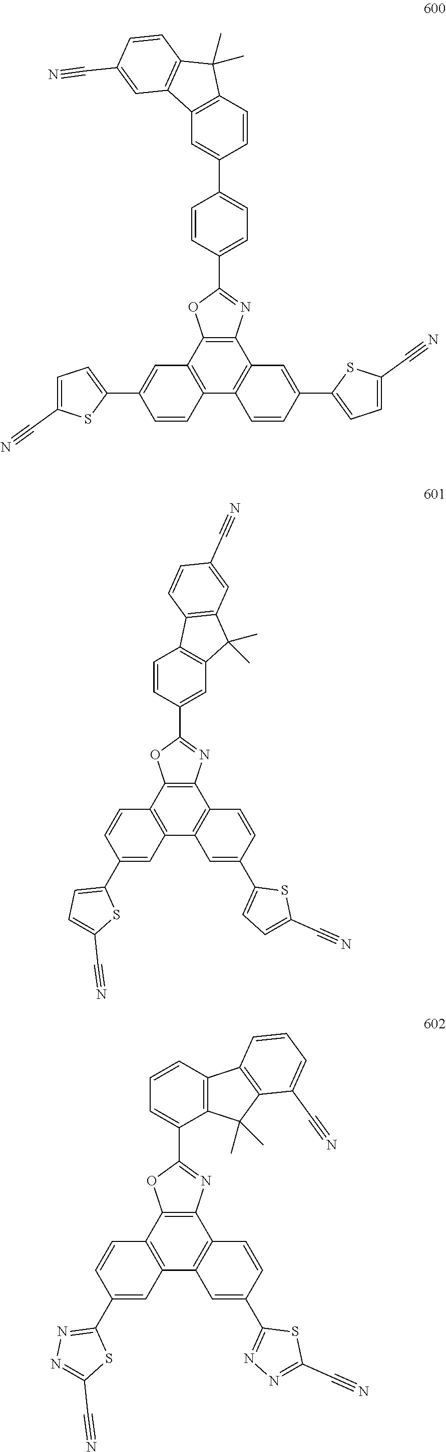 [SMILES: CC1(C)c2ccc(C#N)cc2-c2cc(-c3ccc(-c4nc5c6cc(-c7ccc(C#N)s7)ccc6c6ccc(-c7ccc(C#N)s7)cc6c5o4)cc3)ccc21 ; CC1(C)c2cc(C#N)ccc2-c2ccc(-c3nc4c5ccc(-c6ccc(C#N)s6)cc5c5cc(-c6ccc(C#N)s6)ccc5c4o3)cc21 ; CC1(C)c2c(C#N)cccc2-c2cccc(-c3nc4c5ccc(-c6nnc(C#N)s6)cc5c5cc(-c6nnc(C#N)s6)ccc5c4o3)c21]

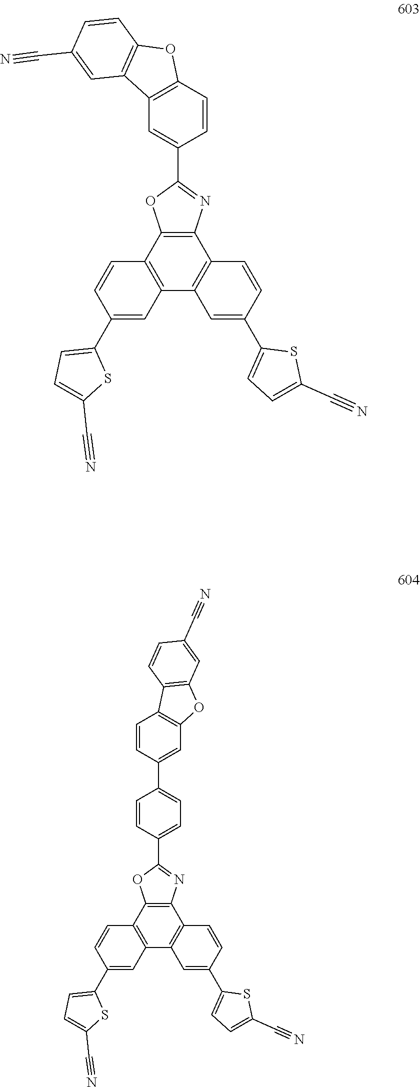 [SMILES: N#Cc1ccc2oc3ccc(-c4nc5c6ccc(-c7ccc(C#N)s7)cc6c6cc(-c7ccc(C#N)s7)ccc6c5o4)cc3c2c1 ; N#Cc1ccc2c(c1)oc1cc(-c3ccc(-c4nc5c6ccc(-c7ccc(C#N)s7)cc6c6cc(-c7ccc(C#N)s7)ccc6c5o4)cc3)ccc12]

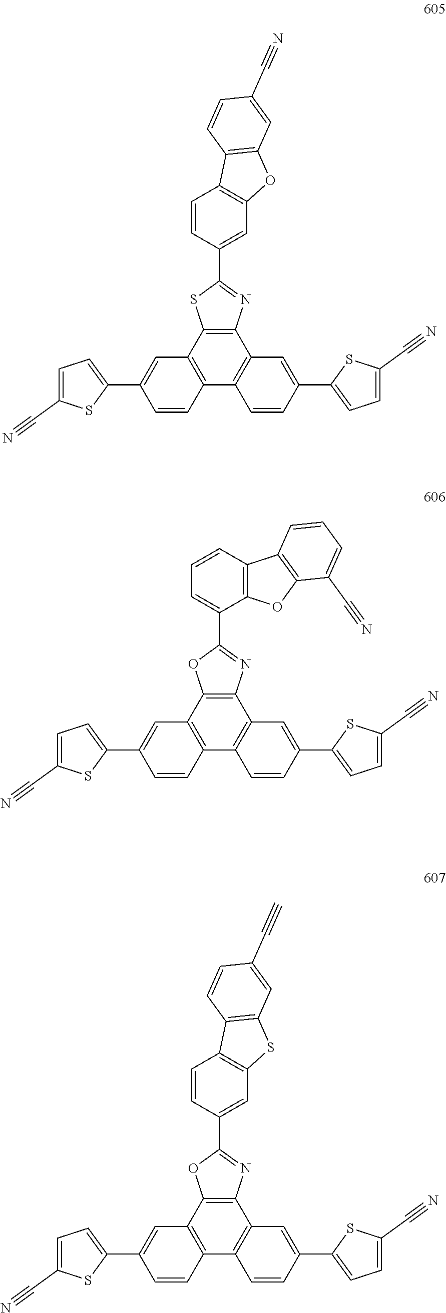 [SMILES: N#Cc1ccc2c(c1)oc1cc(-c3nc4c5cc(-c6ccc(C#N)s6)ccc5c5ccc(-c6ccc(C#N)s6)cc5c4s3)ccc12 ; N#Cc1ccc(-c2ccc3c4ccc(-c5ccc(C#N)s5)cc4c4oc(-c5cccc6c5oc5c(C#N)cccc56)nc4c3c2)s1 ; C#Cc1ccc2c(c1)sc1cc(-c3nc4c5cc(-c6ccc(C#N)s6)ccc5c5ccc(-c6ccc(C#N)s6)cc5c4o3)ccc12]

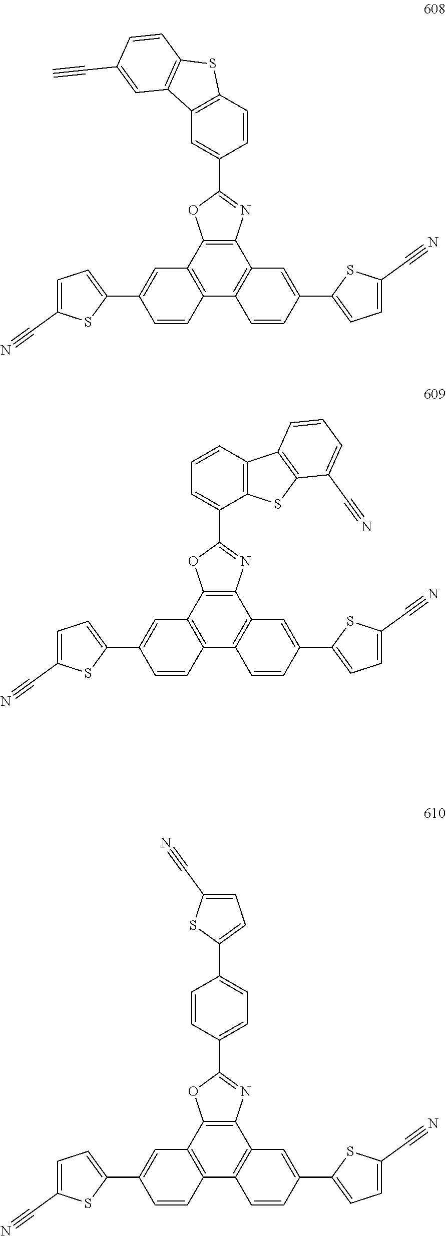 [SMILES: C#Cc1ccc2sc3ccc(-c4nc5c6cc(-c7ccc(C#N)s7)ccc6c6ccc(-c7ccc(C#N)s7)cc6c5o4)cc3c2c1 ; N#Cc1ccc(-c2ccc3c4ccc(-c5ccc(C#N)s5)cc4c4oc(-c5cccc6c5sc5c(C#N)cccc56)nc4c3c2)s1 ; N#Cc1ccc(-c2ccc(-c3nc4c5cc(-c6ccc(C#N)s6)ccc5c5ccc(-c6ccc(C#N)s6)cc5c4o3)cc2)s1]

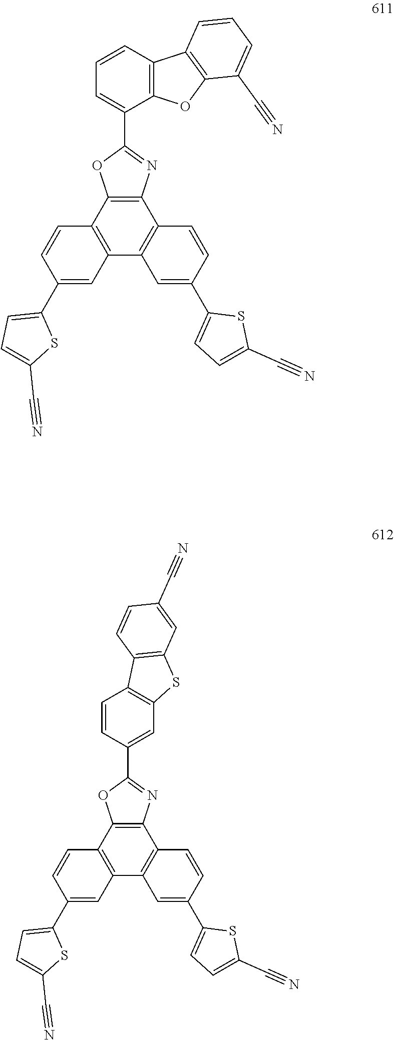 [SMILES: N#Cc1ccc(-c2ccc3c(c2)c2cc(-c4ccc(C#N)s4)ccc2c2oc(-c4cccc5c4oc4c(C#N)cccc45)nc32)s1 ; N#Cc1ccc2c(c1)sc1cc(-c3nc4c5ccc(-c6ccc(C#N)s6)cc5c5cc(-c6ccc(C#N)s6)ccc5c4o3)ccc12]

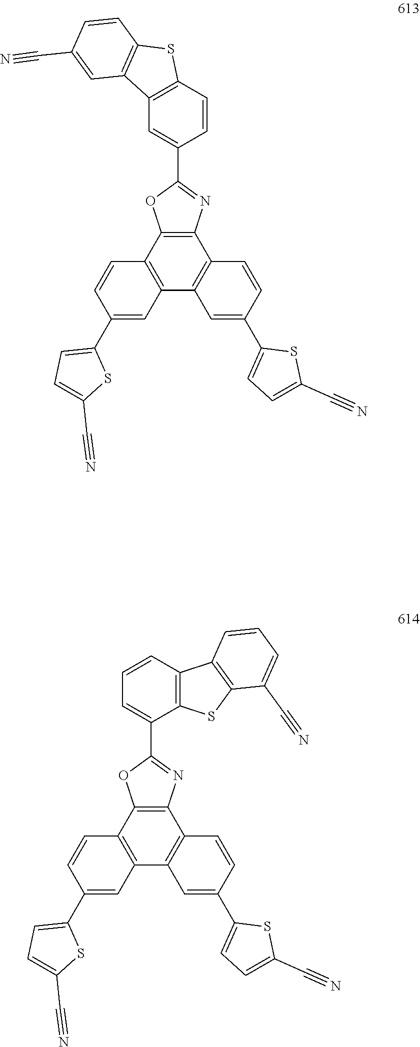 [SMILES: N#Cc1ccc2sc3ccc(-c4nc5c6ccc(-c7ccc(C#N)s7)cc6c6cc(-c7ccc(C#N)s7)ccc6c5o4)cc3c2c1 ; N#Cc1ccc(-c2ccc3c(c2)c2cc(-c4ccc(C#N)s4)ccc2c2oc(-c4cccc5c4sc4c(C#N)cccc45)nc32)s1]

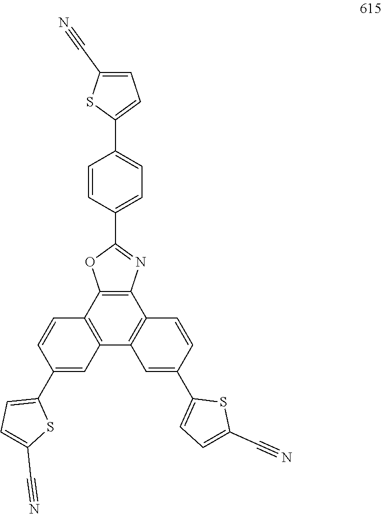 [SMILES: N#Cc1ccc(-c2ccc(-c3nc4c5ccc(-c6ccc(C#N)s6)cc5c5cc(-c6ccc(C#N)s6)ccc5c4o3)cc2)s1]

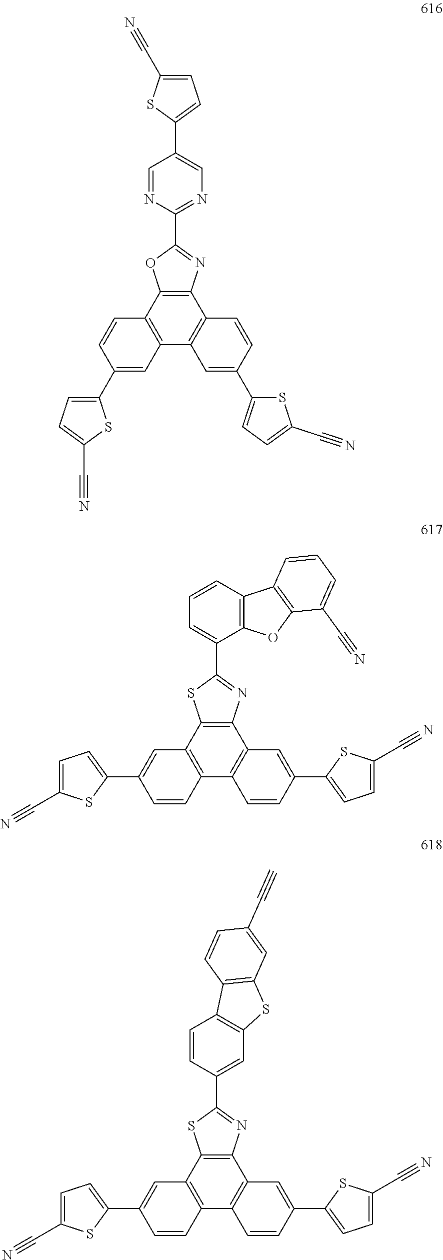 [SMILES: N#Cc1ccc(-c2cnc(-c3nc4c5ccc(-c6ccc(C#N)s6)cc5c5cc(-c6ccc(C#N)s6)ccc5c4o3)nc2)s1 ; N#Cc1ccc(-c2ccc3c4ccc(-c5ccc(C#N)s5)cc4c4sc(-c5cccc6c5oc5c(C#N)cccc56)nc4c3c2)s1 ; C#Cc1ccc2c(c1)sc1cc(-c3nc4c5cc(-c6ccc(C#N)s6)ccc5c5ccc(-c6ccc(C#N)s6)cc5c4s3)ccc12]

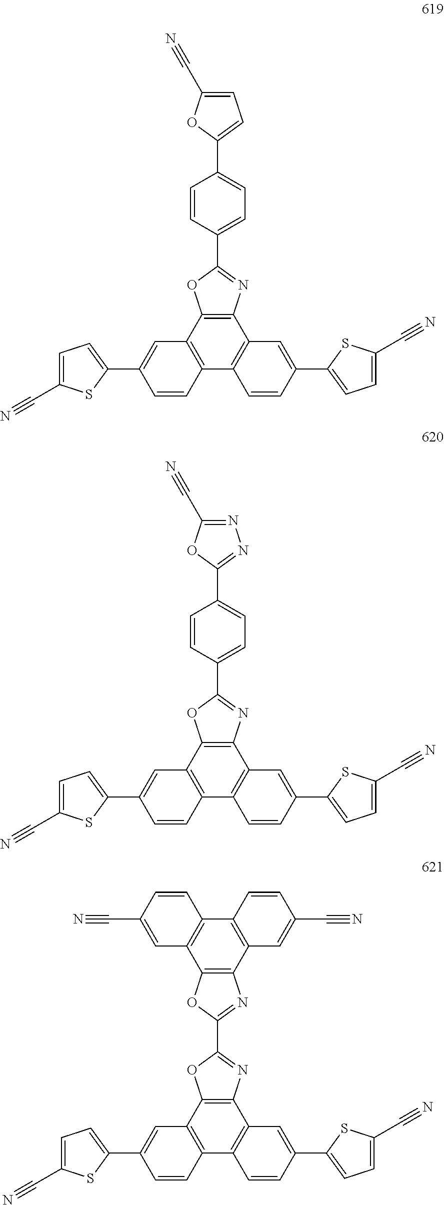 [SMILES: N#Cc1ccc(-c2ccc(-c3nc4c5cc(-c6ccc(C#N)s6)ccc5c5ccc(-c6ccc(C#N)s6)cc5c4o3)cc2)o1 ; N#Cc1nnc(-c2ccc(-c3nc4c5cc(-c6ccc(C#N)s6)ccc5c5ccc(-c6ccc(C#N)s6)cc5c4o3)cc2)o1 ; N#Cc1ccc2c3ccc(C#N)cc3c3oc(-c4nc5c6cc(-c7ccc(C#N)s7)ccc6c6ccc(-c7ccc(C#N)s7)cc6c5o4)nc3c2c1]

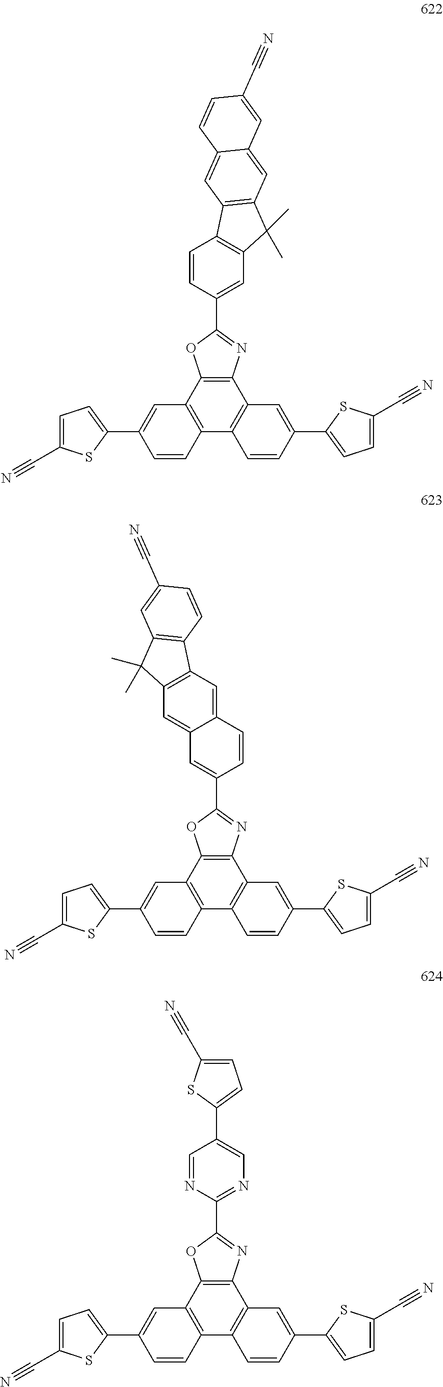 [SMILES: CC1(C)c2cc(-c3nc4c5cc(-c6ccc(C#N)s6)ccc5c5ccc(-c6ccc(C#N)s6)cc5c4o3)ccc2-c2cc3ccc(C#N)cc3cc21 ; CC1(C)c2cc(C#N)ccc2-c2cc3ccc(-c4nc5c6cc(-c7ccc(C#N)s7)ccc6c6ccc(-c7ccc(C#N)s7)cc6c5o4)cc3cc21 ; N#Cc1ccc(-c2cnc(-c3nc4c5cc(-c6ccc(C#N)s6)ccc5c5ccc(-c6ccc(C#N)s6)cc5c4o3)nc2)s1]

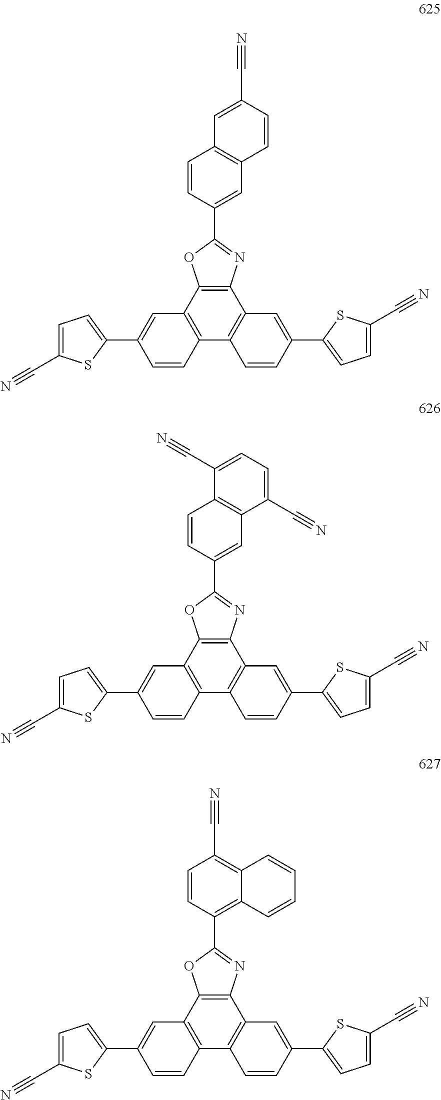 [SMILES: N#Cc1ccc2cc(-c3nc4c5cc(-c6ccc(C#N)s6)ccc5c5ccc(-c6ccc(C#N)s6)cc5c4o3)ccc2c1 ; N#Cc1ccc(-c2ccc3c4ccc(-c5ccc(C#N)s5)cc4c4oc(-c5ccc6c(C#N)ccc(C#N)c6c5)nc4c3c2)s1 ; N#Cc1ccc(-c2ccc3c4ccc(-c5ccc(C#N)s5)cc4c4oc(-c5ccc(C#N)c6ccccc56)nc4c3c2)s1]

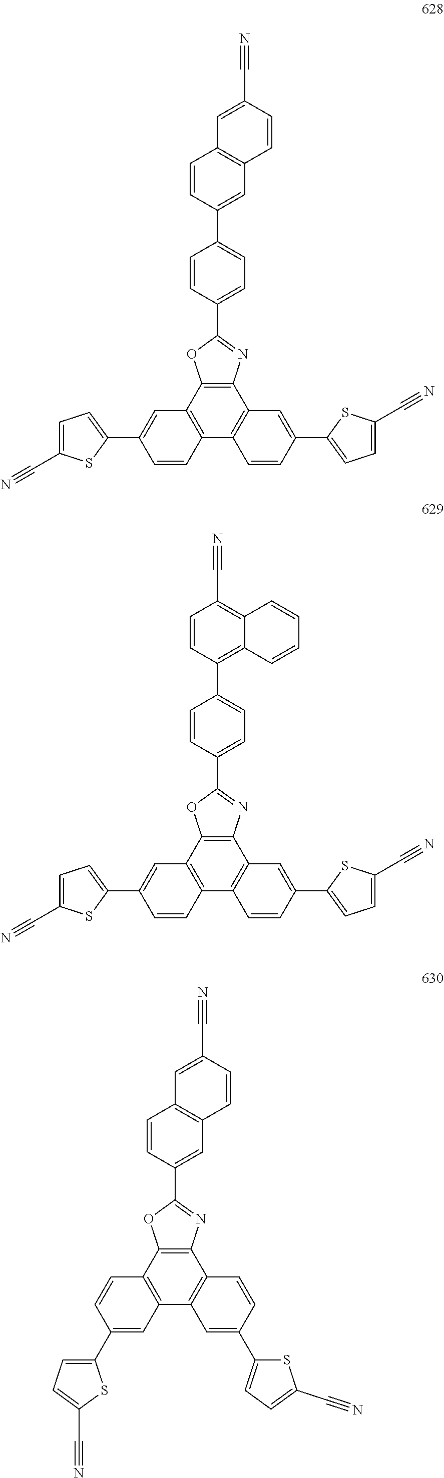 [SMILES: N#Cc1ccc2cc(-c3ccc(-c4nc5c6cc(-c7ccc(C#N)s7)ccc6c6ccc(-c7ccc(C#N)s7)cc6c5o4)cc3)ccc2c1 ; N#Cc1ccc(-c2ccc3c4ccc(-c5ccc(C#N)s5)cc4c4oc(-c5ccc(-c6ccc(C#N)c7ccccc67)cc5)nc4c3c2)s1 ; N#Cc1ccc2cc(-c3nc4c5ccc(-c6ccc(C#N)s6)cc5c5cc(-c6ccc(C#N)s6)ccc5c4o3)ccc2c1]

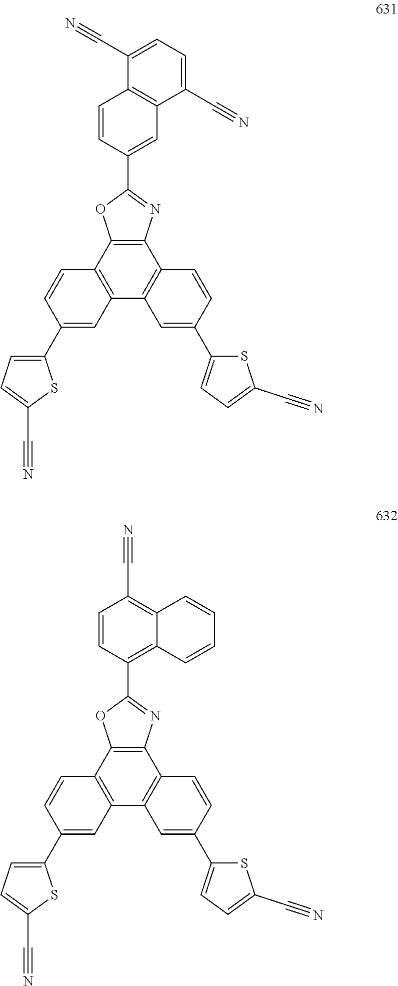 [SMILES: N#Cc1ccc(-c2ccc3c(c2)c2cc(-c4ccc(C#N)s4)ccc2c2oc(-c4ccc5c(C#N)ccc(C#N)c5c4)nc32)s1 ; N#Cc1ccc(-c2ccc3c(c2)c2cc(-c4ccc(C#N)s4)ccc2c2oc(-c4ccc(C#N)c5ccccc45)nc32)s1]

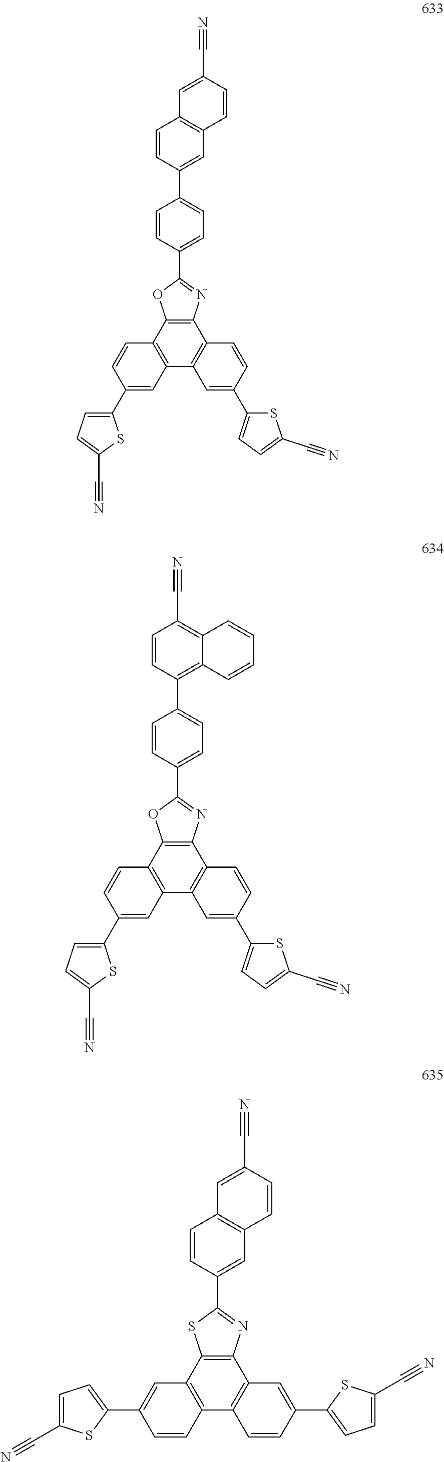 [SMILES: N#Cc1ccc2cc(-c3ccc(-c4nc5c6ccc(-c7ccc(C#N)s7)cc6c6cc(-c7ccc(C#N)s7)ccc6c5o4)cc3)ccc2c1 ; N#Cc1ccc(-c2ccc3c(c2)c2cc(-c4ccc(C#N)s4)ccc2c2oc(-c4ccc(-c5ccc(C#N)c6ccccc56)cc4)nc32)s1 ; N#Cc1ccc2cc(-c3nc4c5cc(-c6ccc(C#N)s6)ccc5c5ccc(-c6ccc(C#N)s6)cc5c4s3)ccc2c1]

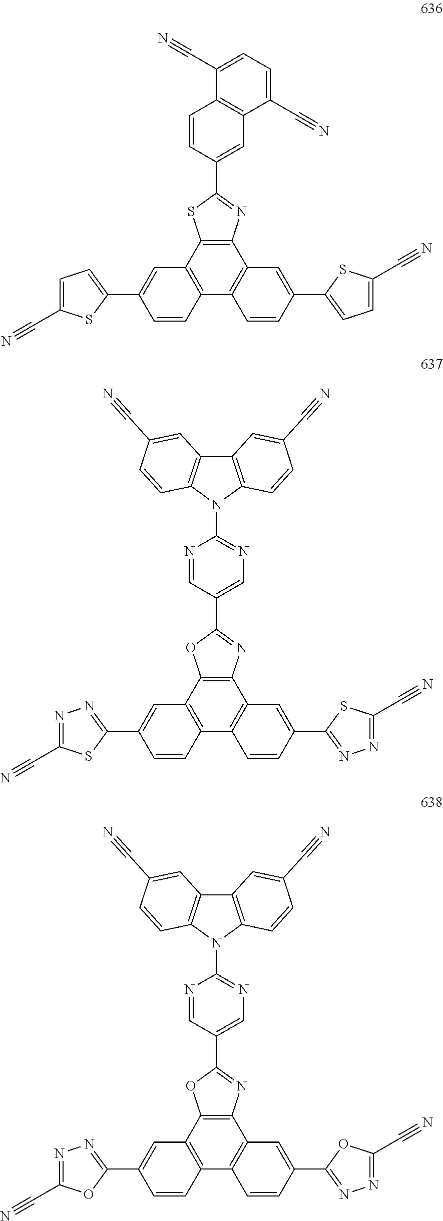 [SMILES: N#Cc1ccc(-c2ccc3c4ccc(-c5ccc(C#N)s5)cc4c4sc(-c5ccc6c(C#N)ccc(C#N)c6c5)nc4c3c2)s1 ; N#Cc1ccc2c(c1)c1cc(C#N)ccc1n2-c1ncc(-c2nc3c4cc(-c5nnc(C#N)s5)ccc4c4ccc(-c5nnc(C#N)s5)cc4c3o2)cn1 ; N#Cc1ccc2c(c1)c1cc(C#N)ccc1n2-c1ncc(-c2nc3c4cc(-c5nnc(C#N)o5)ccc4c4ccc(-c5nnc(C#N)o5)cc4c3o2)cn1]

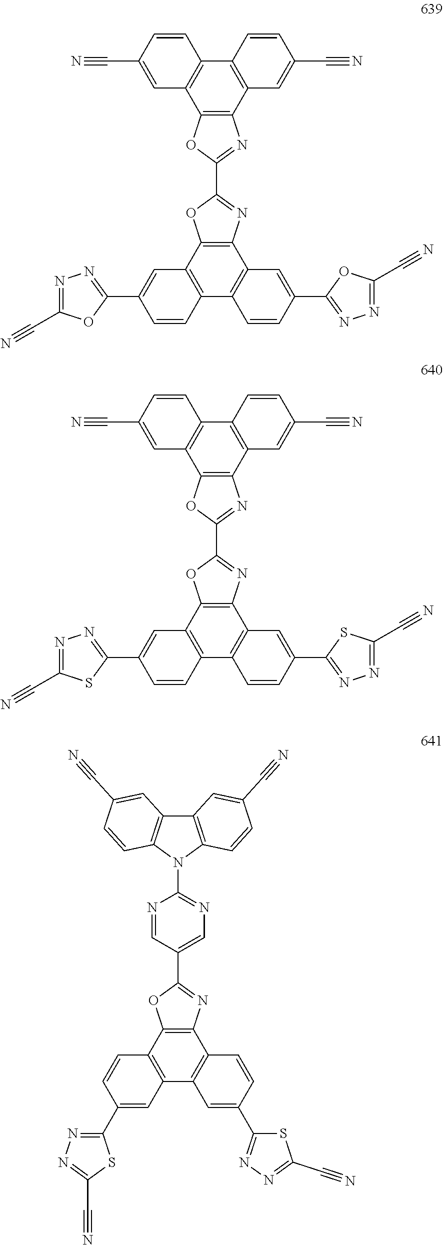 [SMILES: N#Cc1ccc2c3ccc(C#N)cc3c3oc(-c4nc5c6cc(-c7nnc(C#N)o7)ccc6c6ccc(-c7nnc(C#N)o7)cc6c5o4)nc3c2c1 ; N#Cc1ccc2c3ccc(C#N)cc3c3oc(-c4nc5c6cc(-c7nnc(C#N)s7)ccc6c6ccc(-c7nnc(C#N)s7)cc6c5o4)nc3c2c1 ; N#Cc1ccc2c(c1)c1cc(C#N)ccc1n2-c1ncc(-c2nc3c4ccc(-c5nnc(C#N)s5)cc4c4cc(-c5nnc(C#N)s5)ccc4c3o2)cn1]

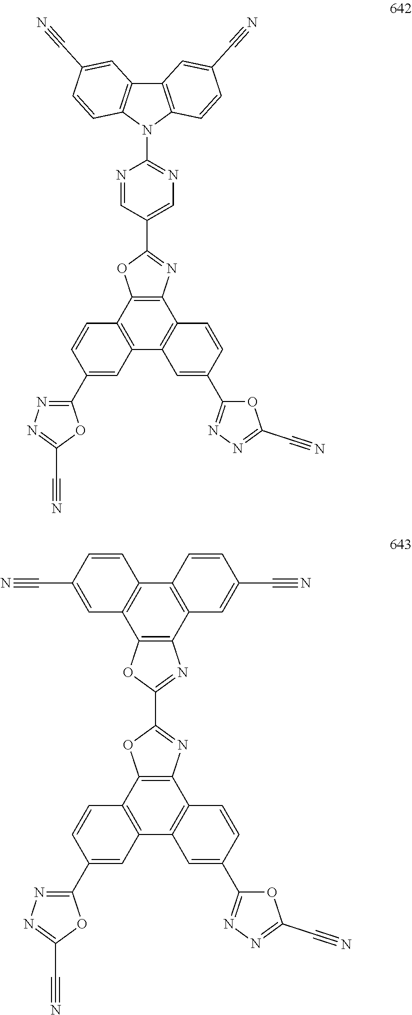 [SMILES: N#Cc1ccc2c(c1)c1cc(C#N)ccc1n2-c1ncc(-c2nc3c4ccc(-c5nnc(C#N)o5)cc4c4cc(-c5nnc(C#N)o5)ccc4c3o2)cn1 ; N#Cc1ccc2c3ccc(C#N)cc3c3oc(-c4nc5c6ccc(-c7nnc(C#N)o7)cc6c6cc(-c7nnc(C#N)o7)ccc6c5o4)nc3c2c1]

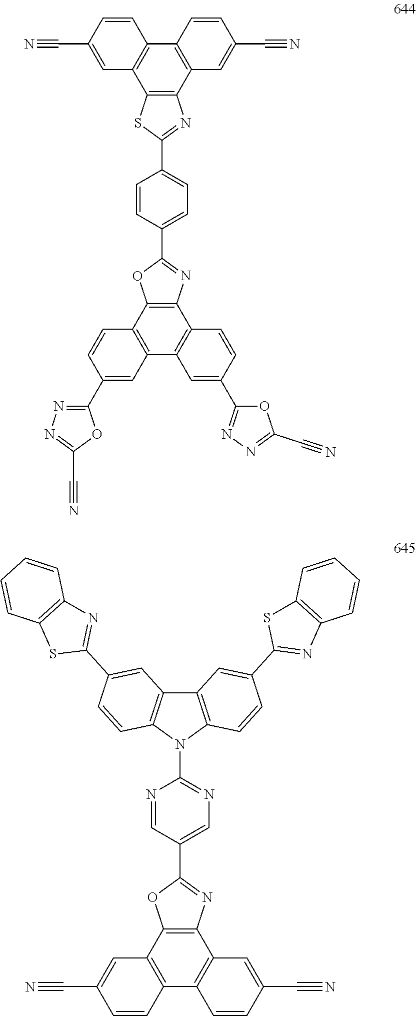 [SMILES: N#Cc1ccc2c3ccc(C#N)cc3c3sc(-c4ccc(-c5nc6c7ccc(-c8nnc(C#N)o8)cc7c7cc(-c8nnc(C#N)o8)ccc7c6o5)cc4)nc3c2c1 ; N#Cc1ccc2c3ccc(C#N)cc3c3oc(-c4cnc(-n5c6ccc(-c7nc8ccccc8s7)cc6c6cc(-c7nc8ccccc8s7)ccc65)nc4)nc3c2c1]

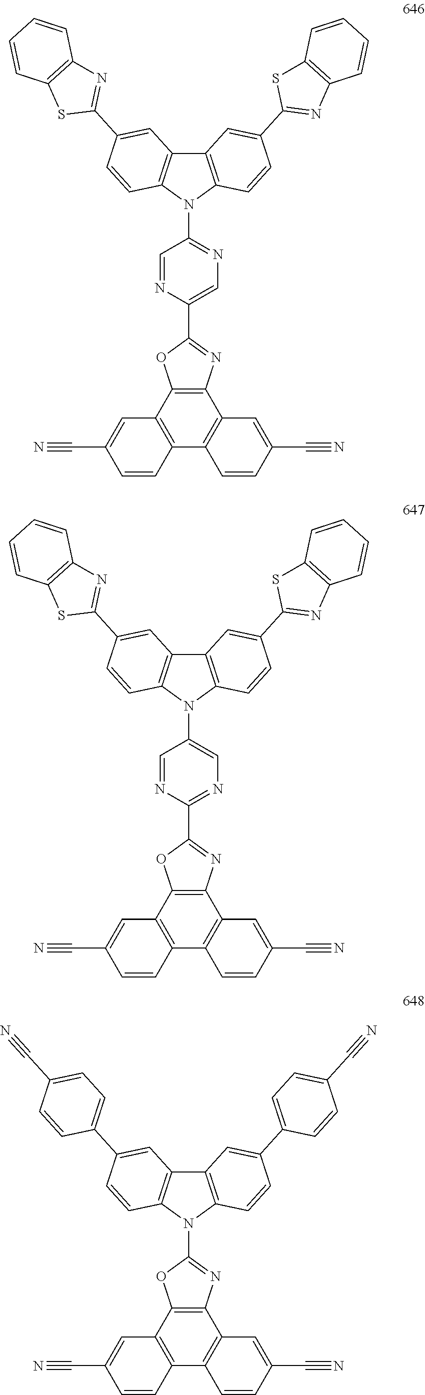 [SMILES: N#Cc1ccc2c3ccc(C#N)cc3c3oc(-c4cnc(-n5c6ccc(-c7nc8ccccc8s7)cc6c6cc(-c7nc8ccccc8s7)ccc65)cn4)nc3c2c1 ; N#Cc1ccc2c3ccc(C#N)cc3c3oc(-c4ncc(-n5c6ccc(-c7nc8ccccc8s7)cc6c6cc(-c7nc8ccccc8s7)ccc65)cn4)nc3c2c1 ; N#Cc1ccc(-c2ccc3c(c2)c2cc(-c4ccc(C#N)cc4)ccc2n3-c2nc3c4cc(C#N)ccc4c4ccc(C#N)cc4c3o2)cc1]

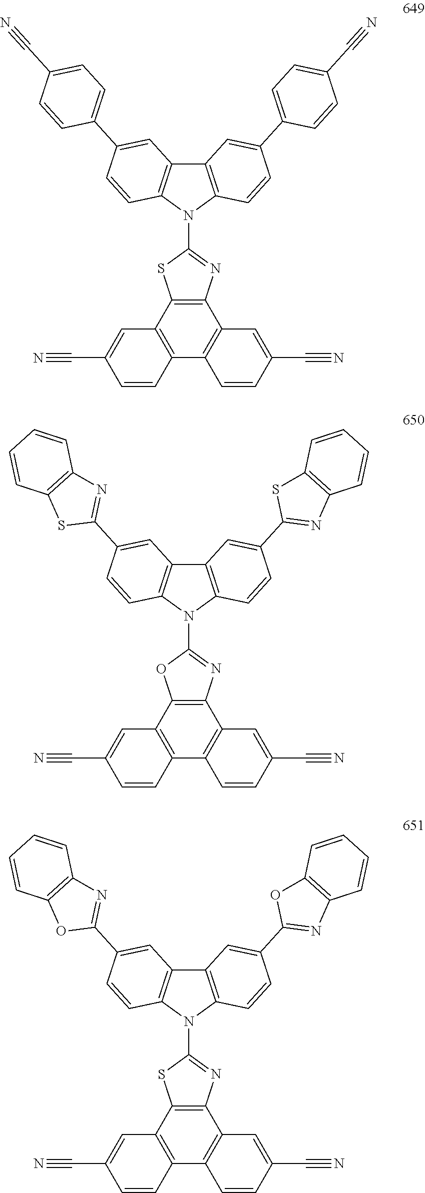 [SMILES: N#Cc1ccc(-c2ccc3c(c2)c2cc(-c4ccc(C#N)cc4)ccc2n3-c2nc3c4cc(C#N)ccc4c4ccc(C#N)cc4c3s2)cc1 ; N#Cc1ccc2c3ccc(C#N)cc3c3oc(-n4c5ccc(-c6nc7ccccc7s6)cc5c5cc(-c6nc7ccccc7s6)ccc54)nc3c2c1 ; N#Cc1ccc2c3ccc(C#N)cc3c3sc(-n4c5ccc(-c6nc7ccccc7o6)cc5c5cc(-c6nc7ccccc7o6)ccc54)nc3c2c1]

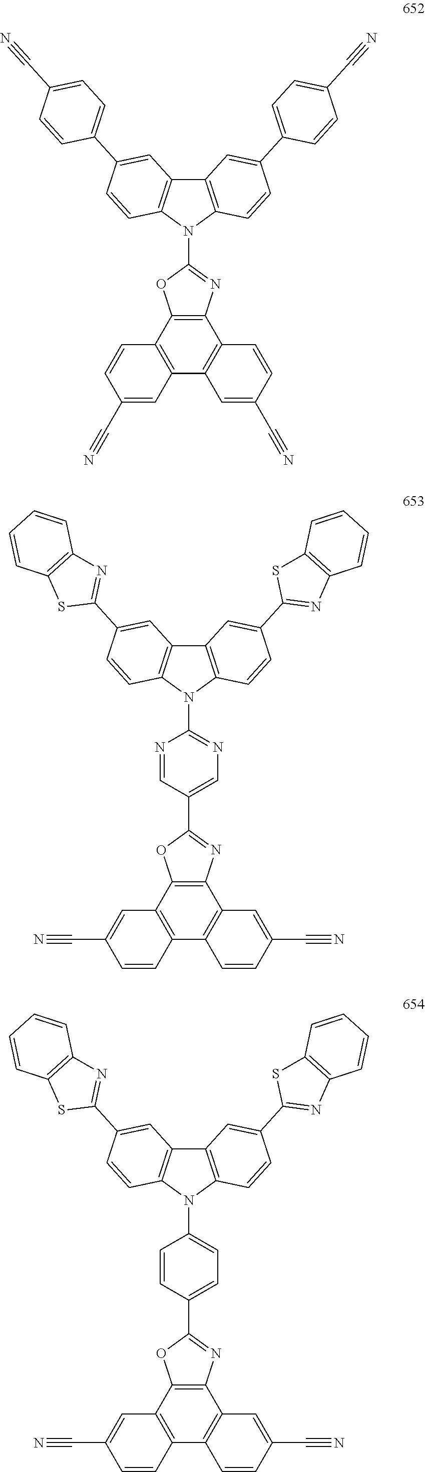 [SMILES: N#Cc1ccc(-c2ccc3c(c2)c2cc(-c4ccc(C#N)cc4)ccc2n3-c2nc3c4ccc(C#N)cc4c4cc(C#N)ccc4c3o2)cc1 ; N#Cc1ccc2c3ccc(C#N)cc3c3oc(-c4cnc(-n5c6ccc(-c7nc8ccccc8s7)cc6c6cc(-c7nc8ccccc8s7)ccc65)nc4)nc3c2c1 ; N#Cc1ccc2c3ccc(C#N)cc3c3oc(-c4ccc(-n5c6ccc(-c7nc8ccccc8s7)cc6c6cc(-c7nc8ccccc8s7)ccc65)cc4)nc3c2c1]

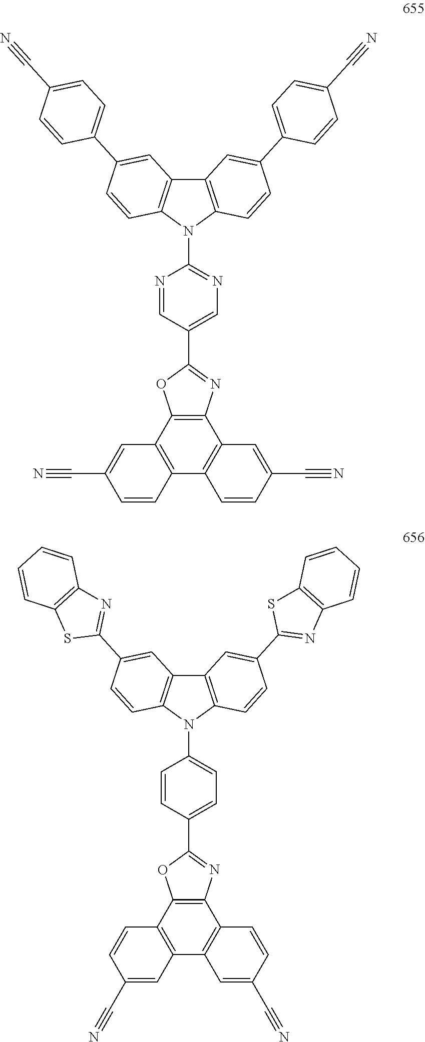 [SMILES: N#Cc1ccc(-c2ccc3c(c2)c2cc(-c4ccc(C#N)cc4)ccc2n3-c2ncc(-c3nc4c5cc(C#N)ccc5c5ccc(C#N)cc5c4o3)cn2)cc1 ; N#Cc1ccc2c(c1)c1cc(C#N)ccc1c1oc(-c3ccc(-n4c5ccc(-c6nc7ccccc7s6)cc5c5cc(-c6nc7ccccc7s6)ccc54)cc3)nc21]

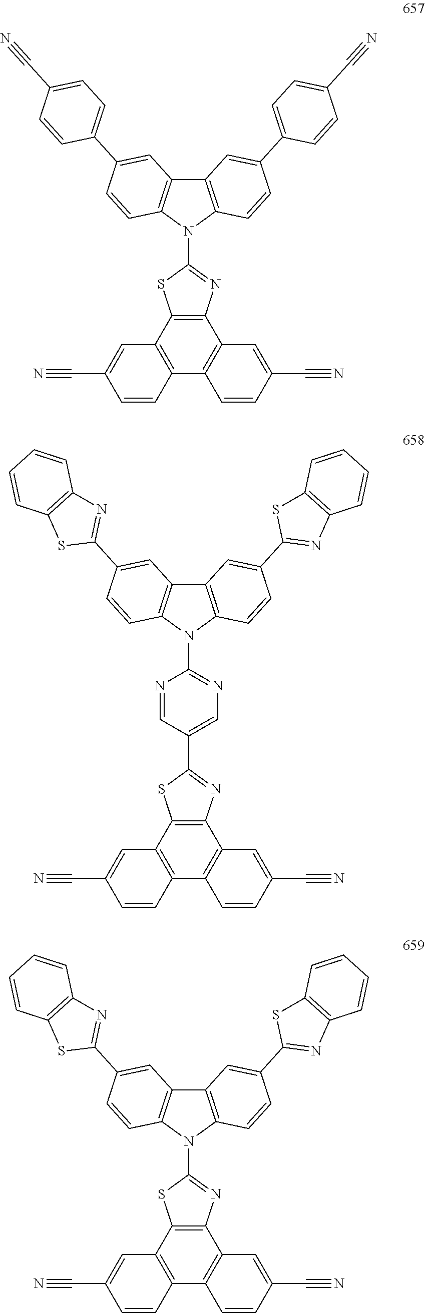 [SMILES: N#Cc1ccc(-c2ccc3c(c2)c2cc(-c4ccc(C#N)cc4)ccc2n3-c2nc3c4cc(C#N)ccc4c4ccc(C#N)cc4c3s2)cc1 ; N#Cc1ccc2c3ccc(C#N)cc3c3sc(-c4cnc(-n5c6ccc(-c7nc8ccccc8s7)cc6c6cc(-c7nc8ccccc8s7)ccc65)nc4)nc3c2c1 ; N#Cc1ccc2c3ccc(C#N)cc3c3sc(-n4c5ccc(-c6nc7ccccc7s6)cc5c5cc(-c6nc7ccccc7s6)ccc54)nc3c2c1]

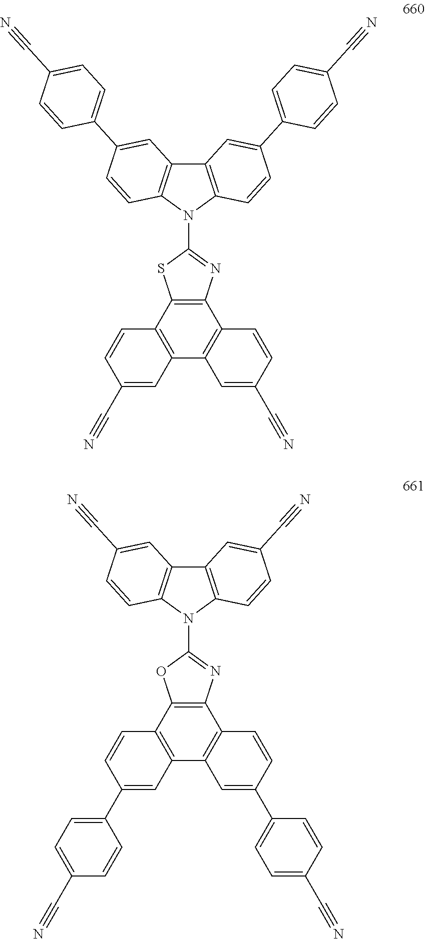 [SMILES: N#Cc1ccc(-c2ccc3c(c2)c2cc(-c4ccc(C#N)cc4)ccc2n3-c2nc3c4ccc(C#N)cc4c4cc(C#N)ccc4c3s2)cc1 ; N#Cc1ccc(-c2ccc3c(c2)c2cc(-c4ccc(C#N)cc4)ccc2c2oc(-n4c5ccc(C#N)cc5c5cc(C#N)ccc54)nc32)cc1]

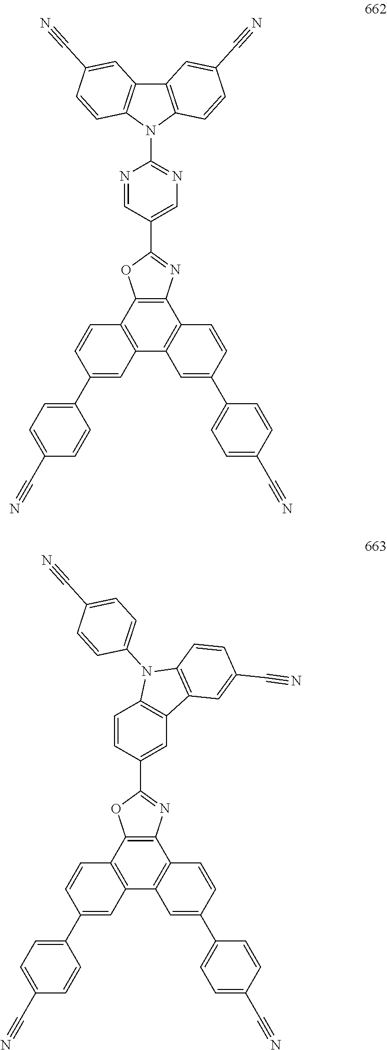 [SMILES: N#Cc1ccc(-c2ccc3c(c2)c2cc(-c4ccc(C#N)cc4)ccc2c2oc(-c4cnc(-n5c6ccc(C#N)cc6c6cc(C#N)ccc65)nc4)nc32)cc1 ; N#Cc1ccc(-c2ccc3c(c2)c2cc(-c4ccc(C#N)cc4)ccc2c2oc(-c4ccc5c(c4)c4cc(C#N)ccc4n5-c4ccc(C#N)cc4)nc32)cc1]

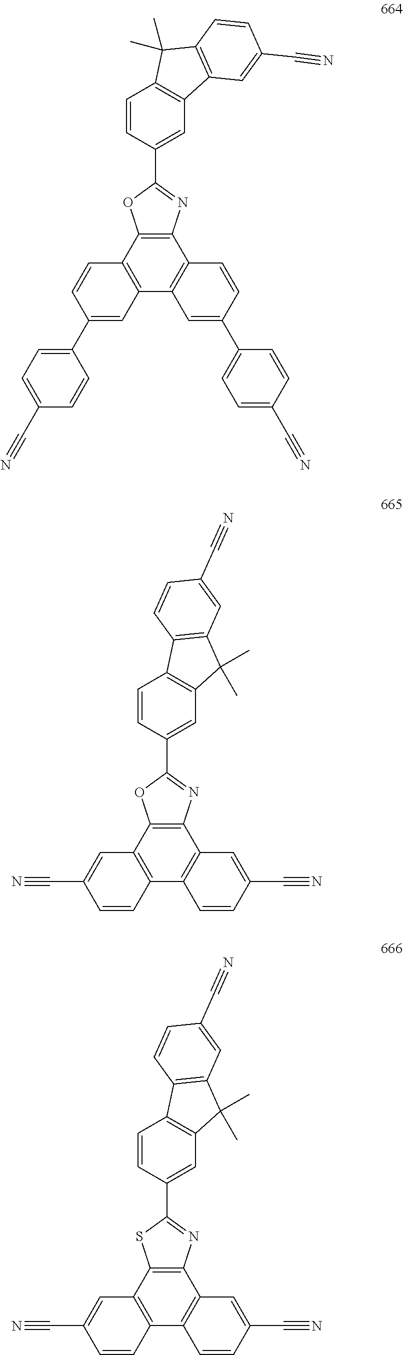 [SMILES: CC1(C)c2ccc(C#N)cc2-c2cc(-c3nc4c5ccc(-c6ccc(C#N)cc6)cc5c5cc(-c6ccc(C#N)cc6)ccc5c4o3)ccc21 ; CC1(C)c2cc(C#N)ccc2-c2ccc(-c3nc4c5cc(C#N)ccc5c5ccc(C#N)cc5c4o3)cc21 ; CC1(C)c2cc(C#N)ccc2-c2ccc(-c3nc4c5cc(C#N)ccc5c5ccc(C#N)cc5c4s3)cc21]

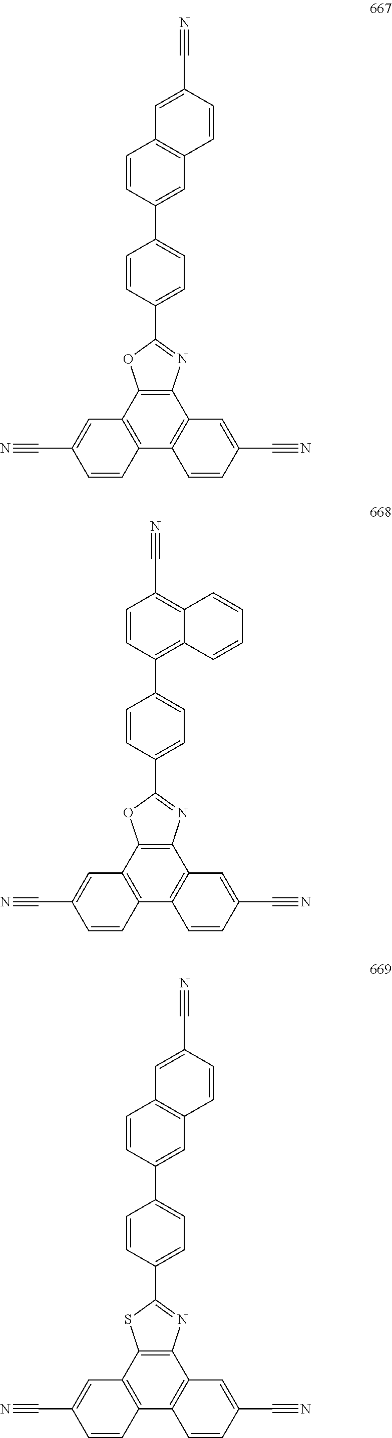 [SMILES: N#Cc1ccc2cc(-c3ccc(-c4nc5c6cc(C#N)ccc6c6ccc(C#N)cc6c5o4)cc3)ccc2c1 ; N#Cc1ccc2c3ccc(C#N)cc3c3oc(-c4ccc(-c5ccc(C#N)c6ccccc56)cc4)nc3c2c1 ; N#Cc1ccc2cc(-c3ccc(-c4nc5c6cc(C#N)ccc6c6ccc(C#N)cc6c5s4)cc3)ccc2c1]

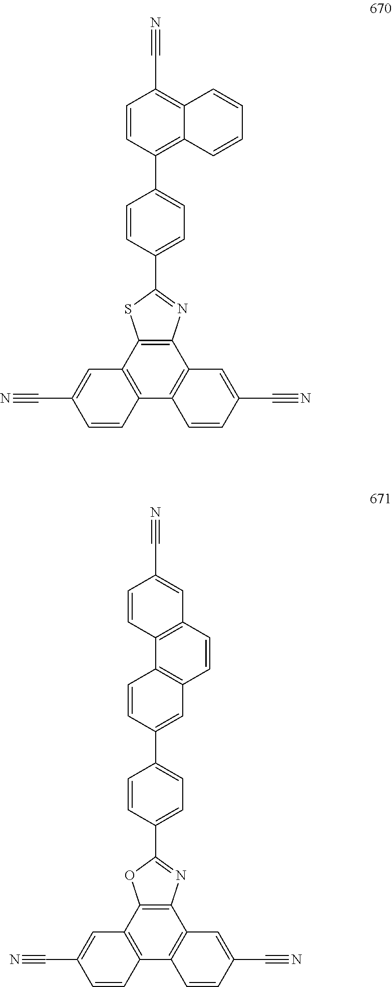 [SMILES: N#Cc1ccc2c3ccc(C#N)cc3c3sc(-c4ccc(-c5ccc(C#N)c6ccccc56)cc4)nc3c2c1 ; N#Cc1ccc2c(ccc3cc(-c4ccc(-c5nc6c7cc(C#N)ccc7c7ccc(C#N)cc7c6o5)cc4)ccc32)c1]

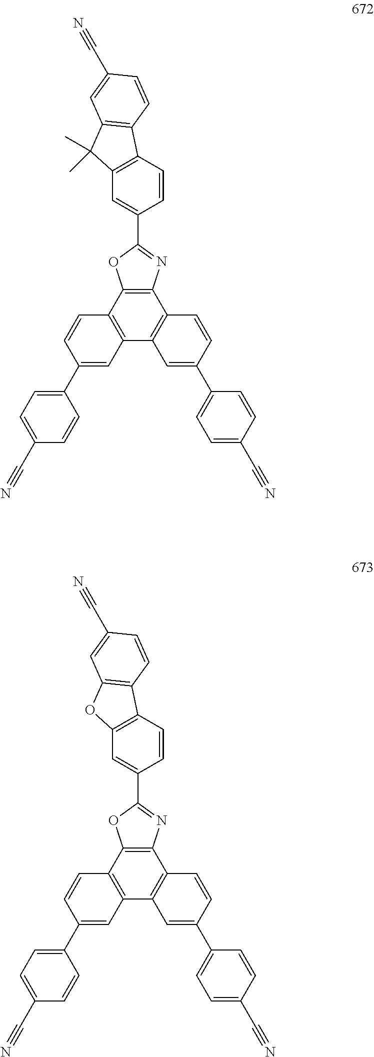 [SMILES: CC1(C)c2cc(C#N)ccc2-c2ccc(-c3nc4c5ccc(-c6ccc(C#N)cc6)cc5c5cc(-c6ccc(C#N)cc6)ccc5c4o3)cc21 ; N#Cc1ccc(-c2ccc3c(c2)c2cc(-c4ccc(C#N)cc4)ccc2c2oc(-c4ccc5c(c4)oc4cc(C#N)ccc45)nc32)cc1]

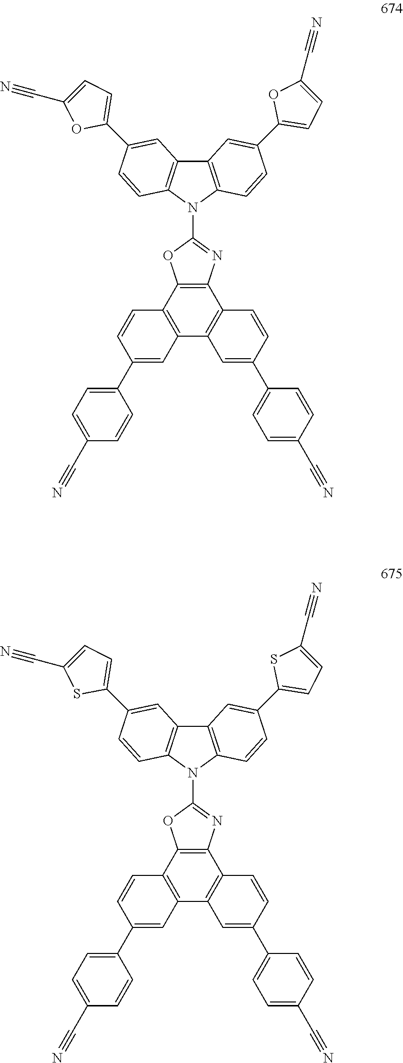 [SMILES: N#Cc1ccc(-c2ccc3c(c2)c2cc(-c4ccc(C#N)cc4)ccc2c2oc(-n4c5ccc(-c6ccc(C#N)o6)cc5c5cc(-c6ccc(C#N)o6)ccc54)nc32)cc1 ; N#Cc1ccc(-c2ccc3c(c2)c2cc(-c4ccc(C#N)cc4)ccc2c2oc(-n4c5ccc(-c6ccc(C#N)s6)cc5c5cc(-c6ccc(C#N)s6)ccc54)nc32)cc1]

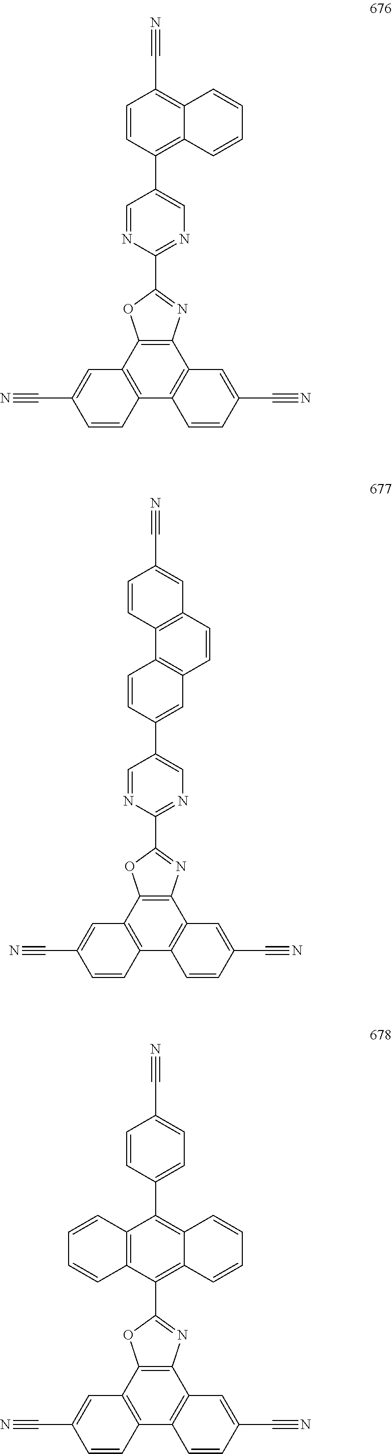 [SMILES: N#Cc1ccc2c3ccc(C#N)cc3c3oc(-c4ncc(-c5ccc(C#N)c6ccccc56)cn4)nc3c2c1 ; N#Cc1ccc2c(ccc3cc(-c4cnc(-c5nc6c7cc(C#N)ccc7c7ccc(C#N)cc7c6o5)nc4)ccc32)c1 ; N#Cc1ccc(-c2c3ccccc3c(-c3nc4c5cc(C#N)ccc5c5ccc(C#N)cc5c4o3)c3ccccc23)cc1]

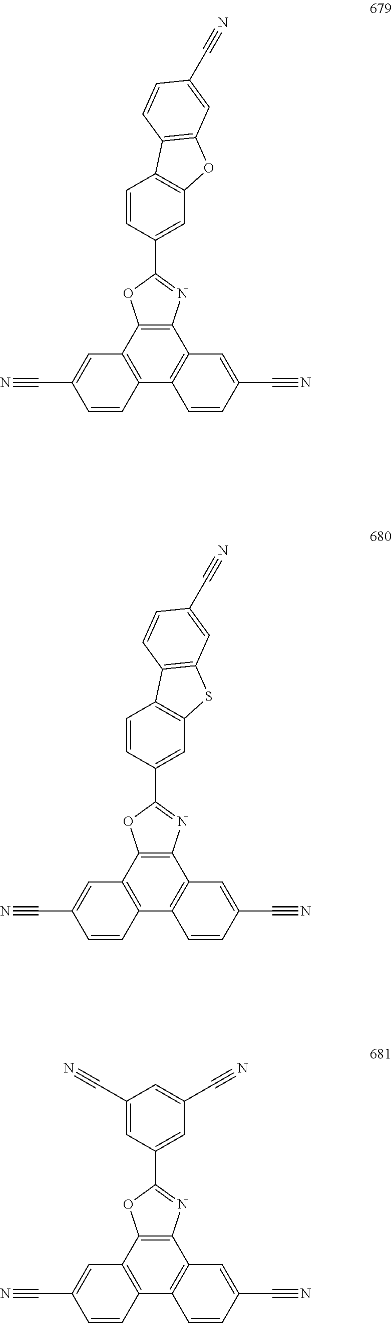 [SMILES: N#Cc1ccc2c(c1)oc1cc(-c3nc4c5cc(C#N)ccc5c5ccc(C#N)cc5c4o3)ccc12 ; N#Cc1ccc2c(c1)sc1cc(-c3nc4c5cc(C#N)ccc5c5ccc(C#N)cc5c4o3)ccc12 ; N#Cc1cc(C#N)cc(-c2nc3c4cc(C#N)ccc4c4ccc(C#N)cc4c3o2)c1]

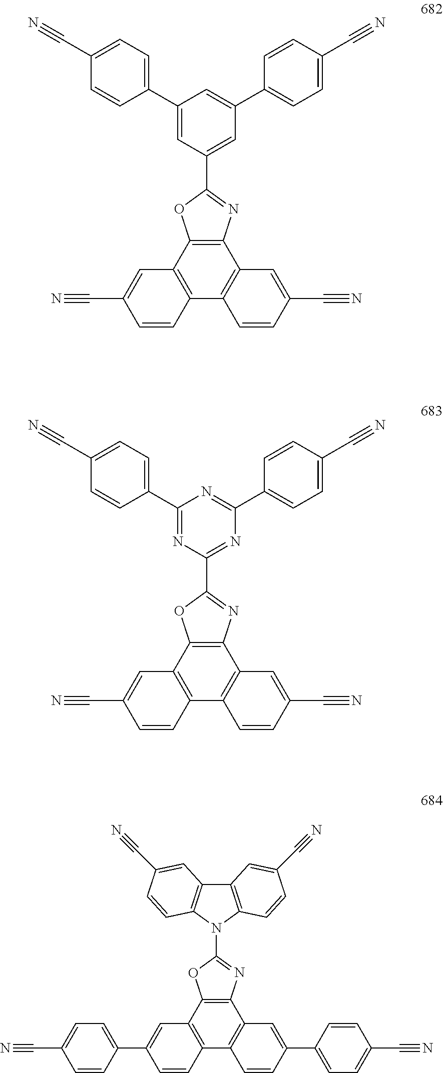 [SMILES: N#Cc1ccc(-c2cc(-c3ccc(C#N)cc3)cc(-c3nc4c5cc(C#N)ccc5c5ccc(C#N)cc5c4o3)c2)cc1 ; N#Cc1ccc(-c2nc(-c3ccc(C#N)cc3)nc(-c3nc4c5cc(C#N)ccc5c5ccc(C#N)cc5c4o3)n2)cc1 ; N#Cc1ccc(-c2ccc3c4ccc(-c5ccc(C#N)cc5)cc4c4oc(-n5c6ccc(C#N)cc6c6cc(C#N)ccc65)nc4c3c2)cc1]

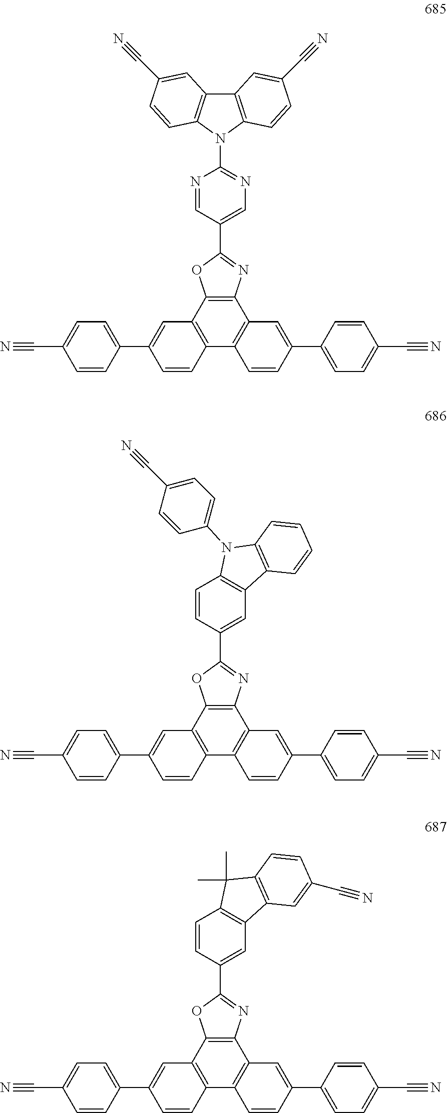 [SMILES: N#Cc1ccc(-c2ccc3c4ccc(-c5ccc(C#N)cc5)cc4c4oc(-c5cnc(-n6c7ccc(C#N)cc7c7cc(C#N)ccc76)nc5)nc4c3c2)cc1 ; N#Cc1ccc(-c2ccc3c4ccc(-c5ccc(C#N)cc5)cc4c4oc(-c5ccc6c(c5)c5ccccc5n6-c5ccc(C#N)cc5)nc4c3c2)cc1 ; CC1(C)c2ccc(C#N)cc2-c2cc(-c3nc4c5cc(-c6ccc(C#N)cc6)ccc5c5ccc(-c6ccc(C#N)cc6)cc5c4o3)ccc21]

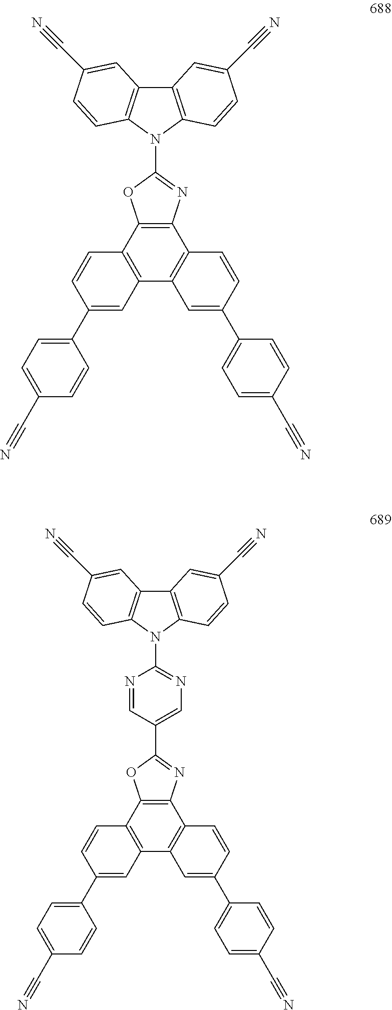 [SMILES: N#Cc1ccc(-c2ccc3c(c2)c2cc(-c4ccc(C#N)cc4)ccc2c2oc(-n4c5ccc(C#N)cc5c5cc(C#N)ccc54)nc32)cc1 ; N#Cc1ccc(-c2ccc3c(c2)c2cc(-c4ccc(C#N)cc4)ccc2c2oc(-c4cnc(-n5c6ccc(C#N)cc6c6cc(C#N)ccc65)nc4)nc32)cc1]

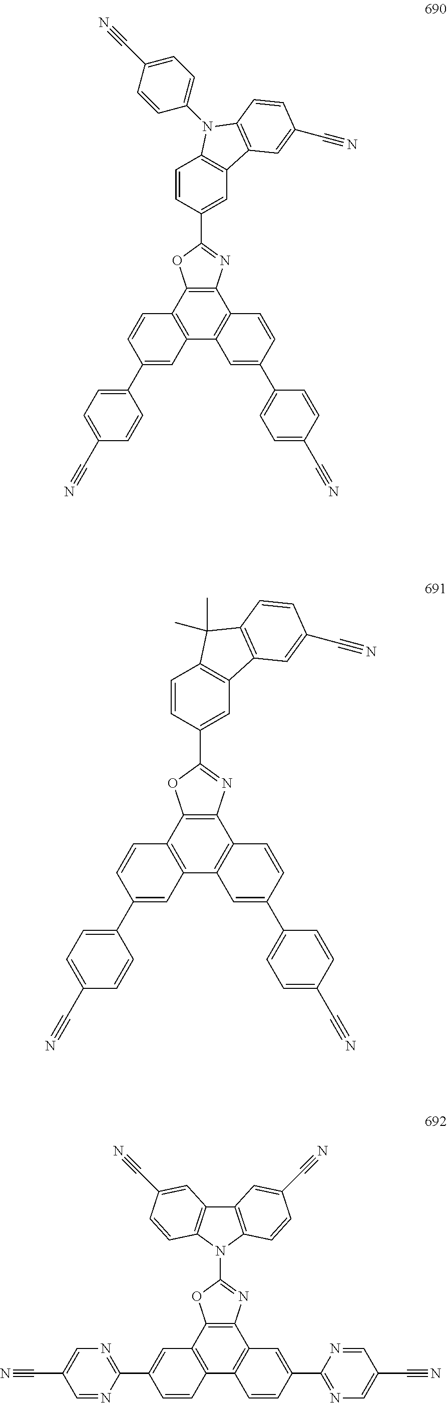 [SMILES: N#Cc1ccc(-c2ccc3c(c2)c2cc(-c4ccc(C#N)cc4)ccc2c2oc(-c4ccc5c(c4)c4cc(C#N)ccc4n5-c4ccc(C#N)cc4)nc32)cc1 ; CC1(C)c2ccc(C#N)cc2-c2cc(-c3nc4c5ccc(-c6ccc(C#N)cc6)cc5c5cc(-c6ccc(C#N)cc6)ccc5c4o3)ccc21 ; N#Cc1cnc(-c2ccc3c4ccc(-c5ncc(C#N)cn5)cc4c4oc(-n5c6ccc(C#N)cc6c6cc(C#N)ccc65)nc4c3c2)nc1]

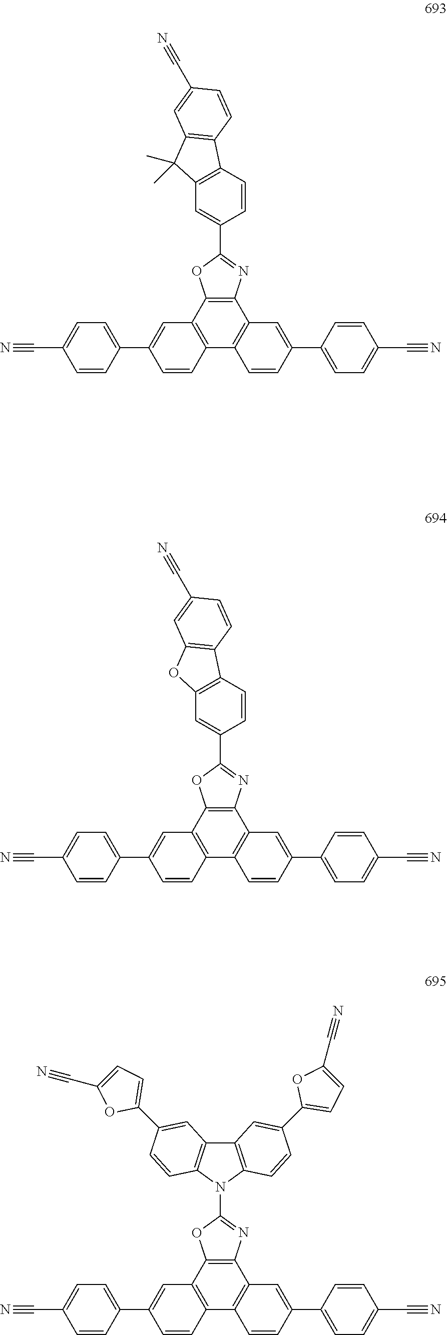 [SMILES: CC1(C)c2cc(C#N)ccc2-c2ccc(-c3nc4c5cc(-c6ccc(C#N)cc6)ccc5c5ccc(-c6ccc(C#N)cc6)cc5c4o3)cc21 ; N#Cc1ccc(-c2ccc3c4ccc(-c5ccc(C#N)cc5)cc4c4oc(-c5ccc6c(c5)oc5cc(C#N)ccc56)nc4c3c2)cc1 ; N#Cc1ccc(-c2ccc3c4ccc(-c5ccc(C#N)cc5)cc4c4oc(-n5c6ccc(-c7ccc(C#N)o7)cc6c6cc(-c7ccc(C#N)o7)ccc65)nc4c3c2)cc1]

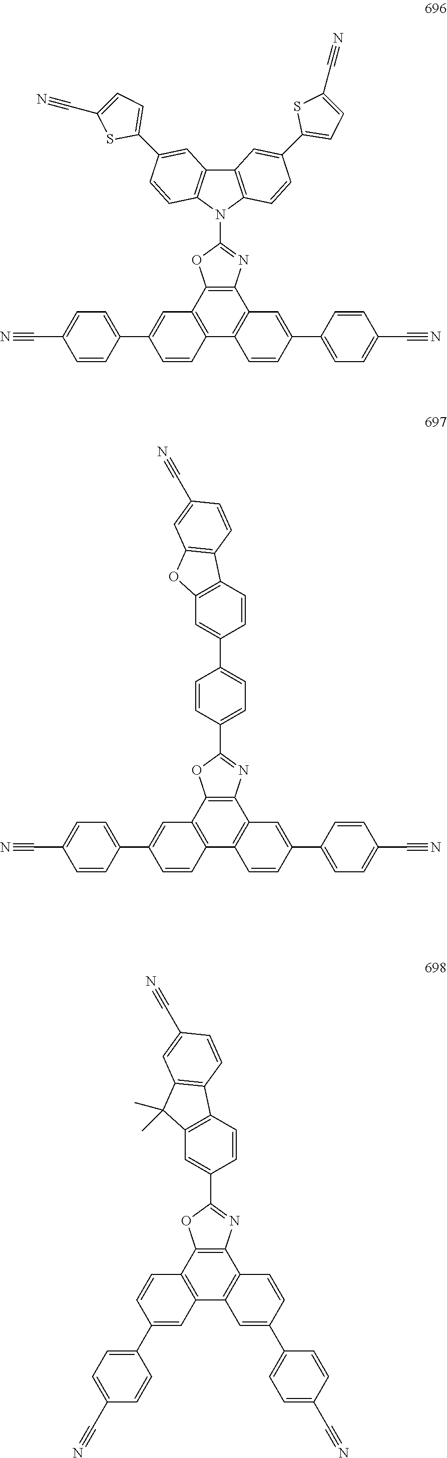 [SMILES: N#Cc1ccc(-c2ccc3c4ccc(-c5ccc(C#N)cc5)cc4c4oc(-n5c6ccc(-c7ccc(C#N)s7)cc6c6cc(-c7ccc(C#N)s7)ccc65)nc4c3c2)cc1 ; N#Cc1ccc(-c2ccc3c4ccc(-c5ccc(C#N)cc5)cc4c4oc(-c5ccc(-c6ccc7c(c6)oc6cc(C#N)ccc67)cc5)nc4c3c2)cc1 ; CC1(C)c2cc(C#N)ccc2-c2ccc(-c3nc4c5ccc(-c6ccc(C#N)cc6)cc5c5cc(-c6ccc(C#N)cc6)ccc5c4o3)cc21]

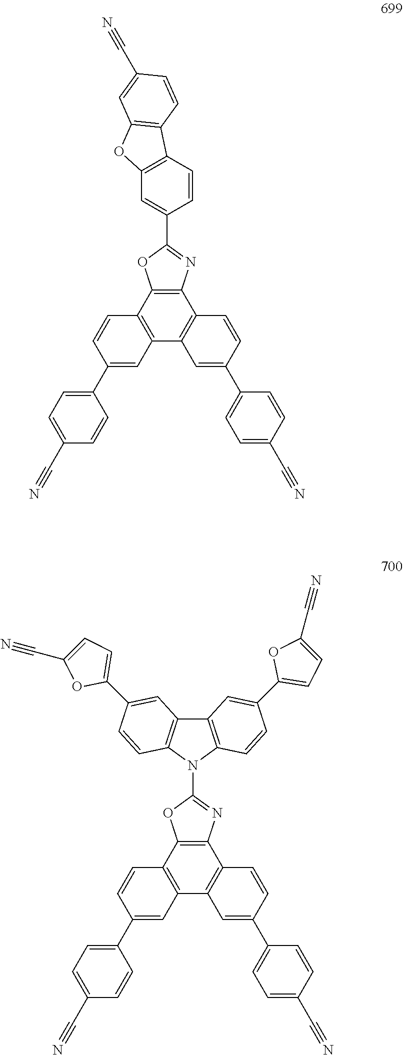 [SMILES: N#Cc1ccc(-c2ccc3c(c2)c2cc(-c4ccc(C#N)cc4)ccc2c2oc(-c4ccc5c(c4)oc4cc(C#N)ccc45)nc32)cc1 ; N#Cc1ccc(-c2ccc3c(c2)c2cc(-c4ccc(C#N)cc4)ccc2c2oc(-n4c5ccc(-c6ccc(C#N)o6)cc5c5cc(-c6ccc(C#N)o6)ccc54)nc32)cc1]

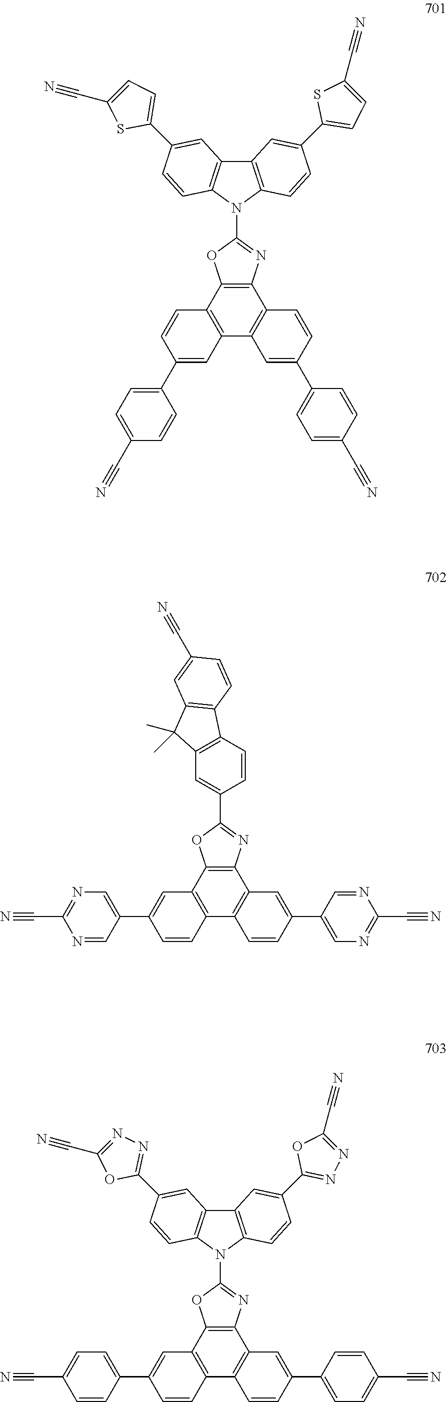 [SMILES: N#Cc1ccc(-c2ccc3c(c2)c2cc(-c4ccc(C#N)cc4)ccc2c2oc(-n4c5ccc(-c6ccc(C#N)s6)cc5c5cc(-c6ccc(C#N)s6)ccc54)nc32)cc1 ; CC1(C)c2cc(C#N)ccc2-c2ccc(-c3nc4c5cc(-c6cnc(C#N)nc6)ccc5c5ccc(-c6cnc(C#N)nc6)cc5c4o3)cc21 ; N#Cc1ccc(-c2ccc3c4ccc(-c5ccc(C#N)cc5)cc4c4oc(-n5c6ccc(-c7nnc(C#N)o7)cc6c6cc(-c7nnc(C#N)o7)ccc65)nc4c3c2)cc1]

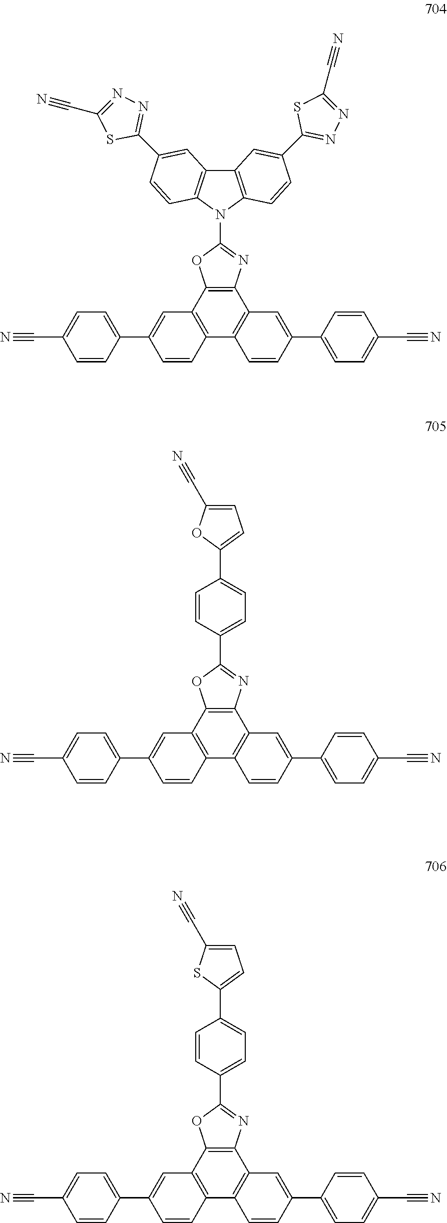 [SMILES: N#Cc1ccc(-c2ccc3c4ccc(-c5ccc(C#N)cc5)cc4c4oc(-n5c6ccc(-c7nnc(C#N)s7)cc6c6cc(-c7nnc(C#N)s7)ccc65)nc4c3c2)cc1 ; N#Cc1ccc(-c2ccc3c4ccc(-c5ccc(C#N)cc5)cc4c4oc(-c5ccc(-c6ccc(C#N)o6)cc5)nc4c3c2)cc1 ; N#Cc1ccc(-c2ccc3c4ccc(-c5ccc(C#N)cc5)cc4c4oc(-c5ccc(-c6ccc(C#N)s6)cc5)nc4c3c2)cc1]

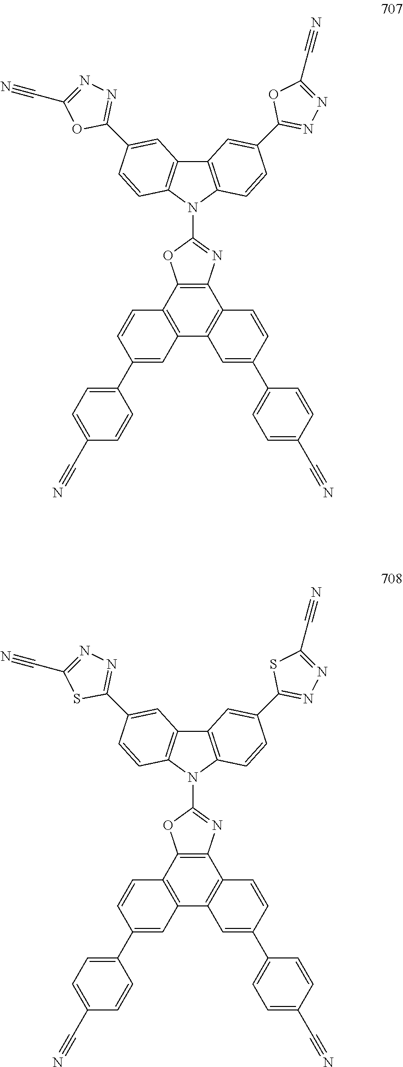 [SMILES: N#Cc1ccc(-c2ccc3c(c2)c2cc(-c4ccc(C#N)cc4)ccc2c2oc(-n4c5ccc(-c6nnc(C#N)o6)cc5c5cc(-c6nnc(C#N)o6)ccc54)nc32)cc1 ; N#Cc1ccc(-c2ccc3c(c2)c2cc(-c4ccc(C#N)cc4)ccc2c2oc(-n4c5ccc(-c6nnc(C#N)s6)cc5c5cc(-c6nnc(C#N)s6)ccc54)nc32)cc1]

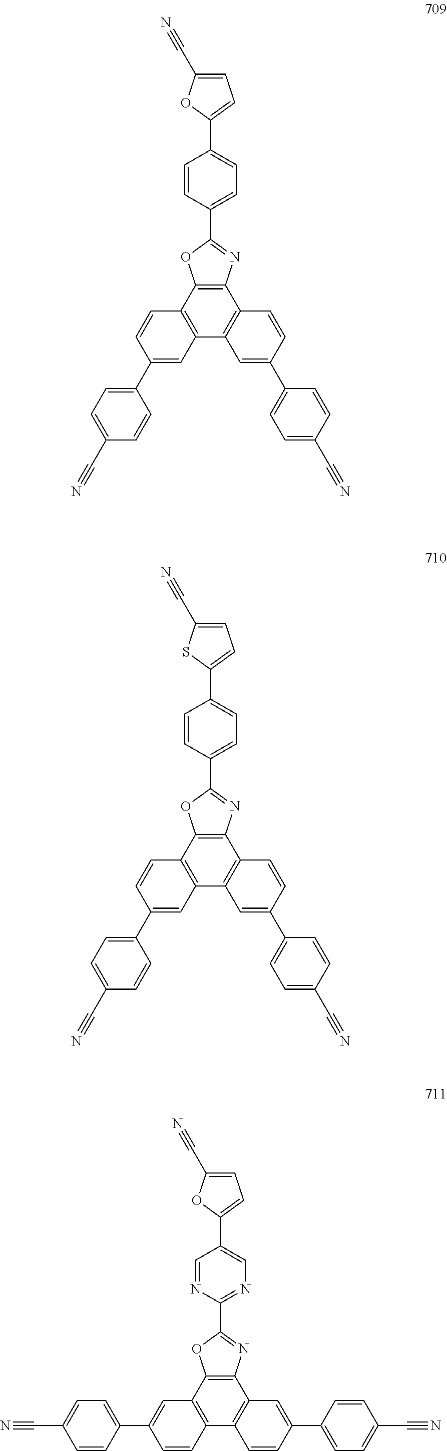

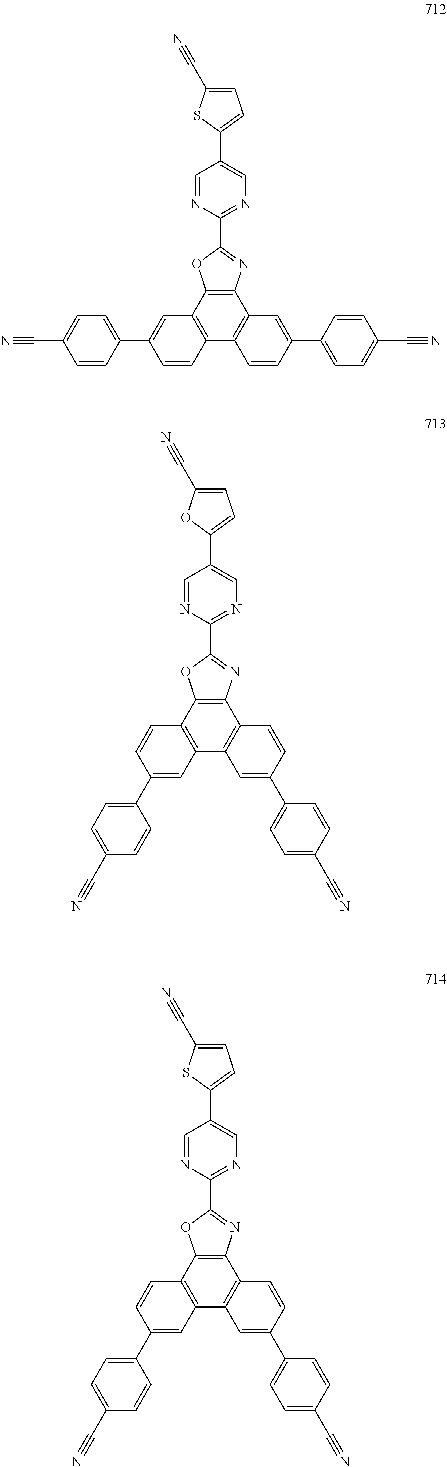

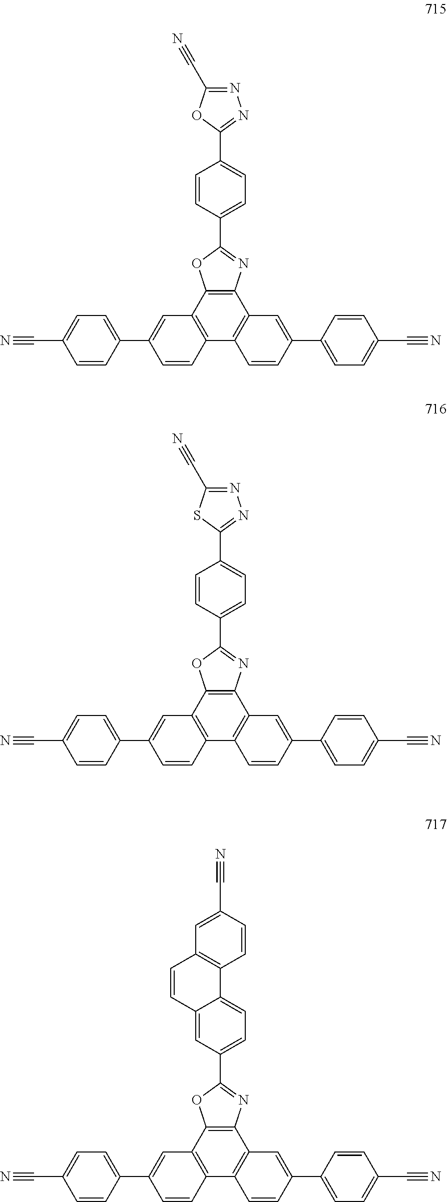

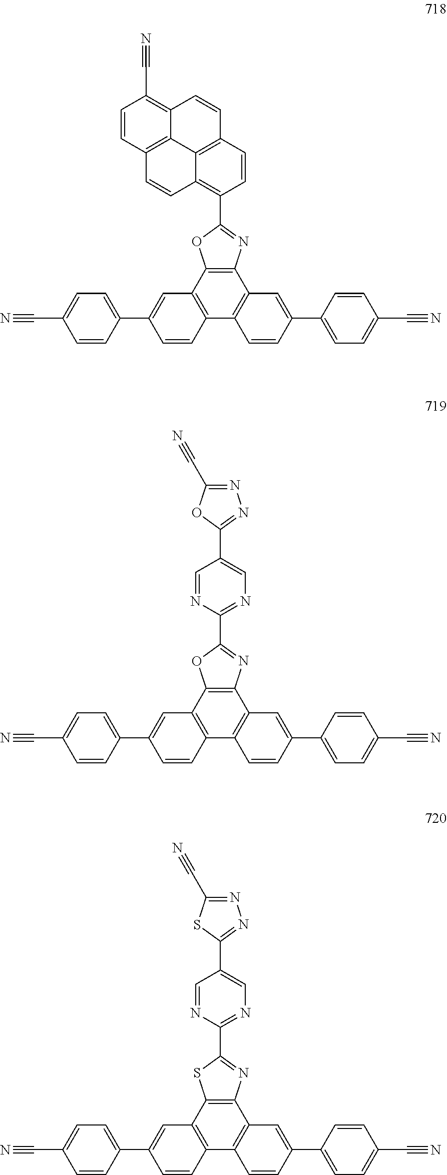

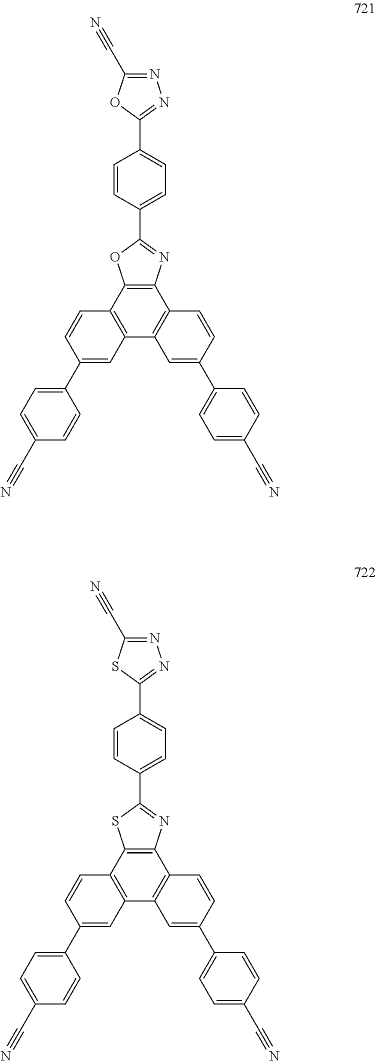

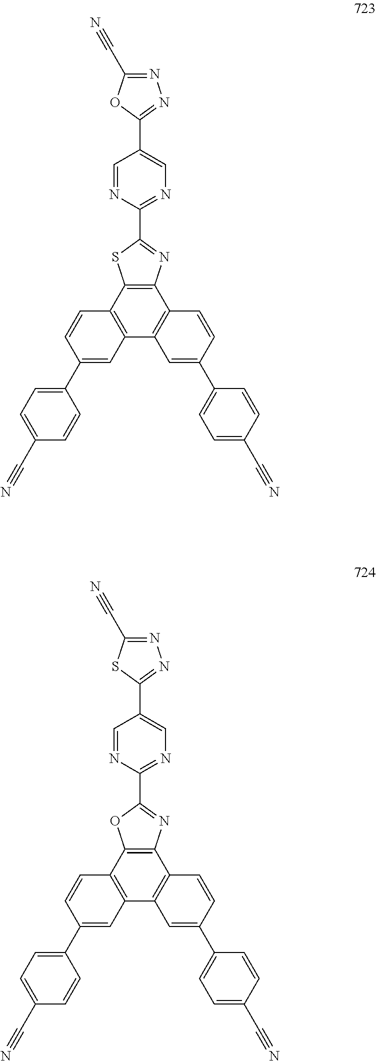

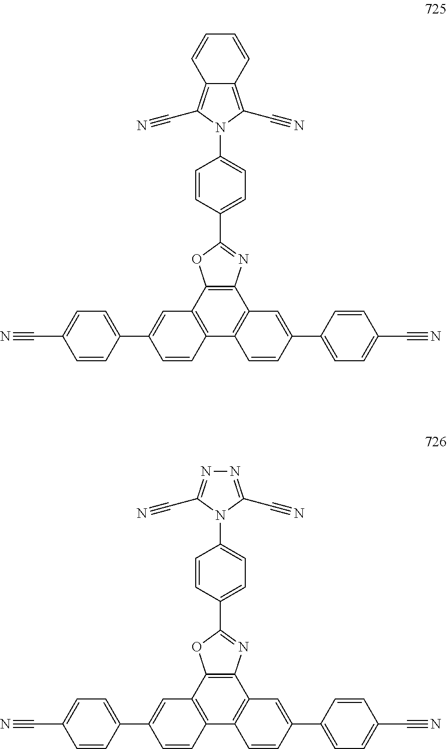

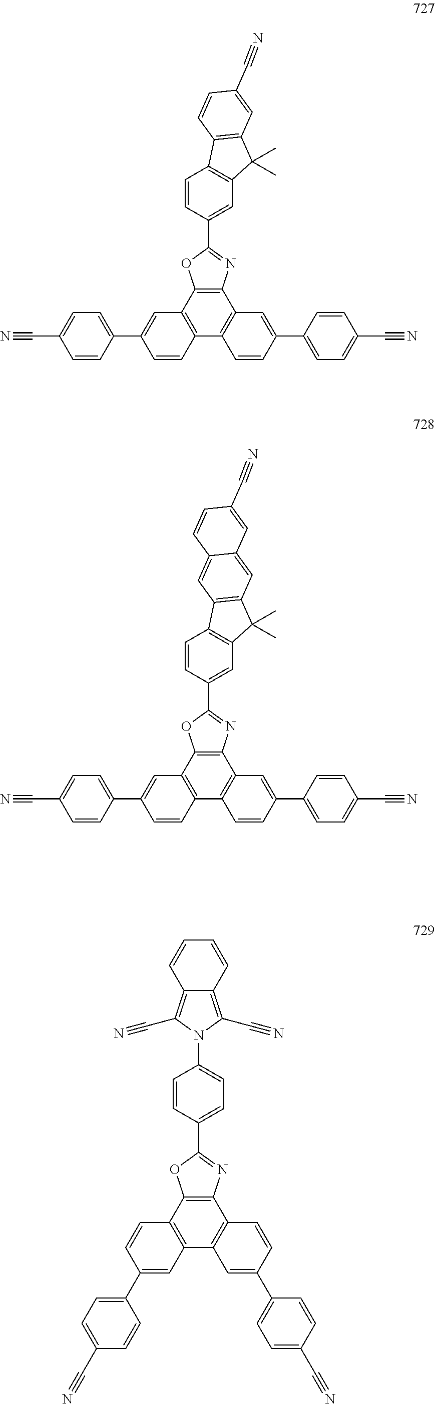 [SMILES: CC1(C)c2cc(C#N)ccc2-c2ccc(-c3nc4c5cc(-c6ccc(C#N)cc6)ccc5c5ccc(-c6ccc(C#N)cc6)cc5c4o3)cc21 ; CC1(C)c2cc(-c3nc4c5cc(-c6ccc(C#N)cc6)ccc5c5ccc(-c6ccc(C#N)cc6)cc5c4o3)ccc2-c2cc3ccc(C#N)cc3cc21 ; N#Cc1ccc(-c2ccc3c(c2)c2cc(-c4ccc(C#N)cc4)ccc2c2oc(-c4ccc(-n5c(C#N)c6ccccc6c5C#N)cc4)nc32)cc1]

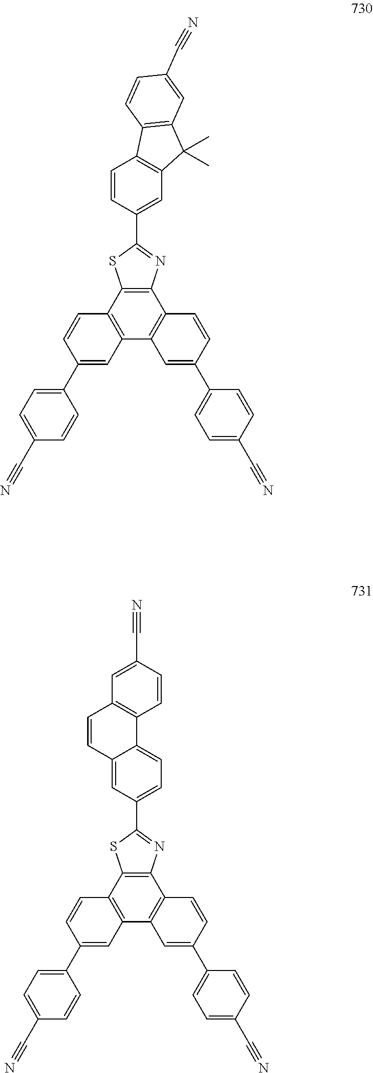 [SMILES: CC1(C)c2cc(C#N)ccc2-c2ccc(-c3nc4c5ccc(-c6ccc(C#N)cc6)cc5c5cc(-c6ccc(C#N)cc6)ccc5c4s3)cc21 ; N#Cc1ccc(-c2ccc3c(c2)c2cc(-c4ccc(C#N)cc4)ccc2c2sc(-c4ccc5c(ccc6cc(C#N)ccc65)c4)nc32)cc1]

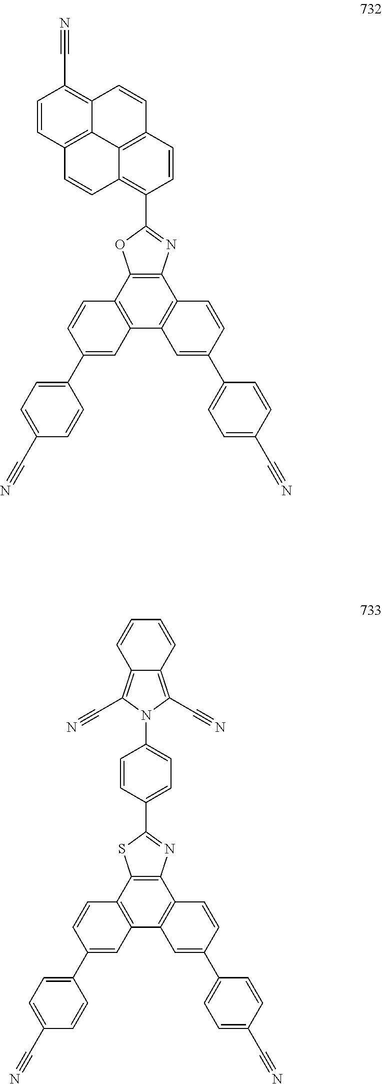 [SMILES: N#Cc1ccc(-c2ccc3c(c2)c2cc(-c4ccc(C#N)cc4)ccc2c2oc(-c4ccc5ccc6c(C#N)ccc7ccc4c5c76)nc32)cc1 ; N#Cc1ccc(-c2ccc3c(c2)c2cc(-c4ccc(C#N)cc4)ccc2c2sc(-c4ccc(-n5c(C#N)c6ccccc6c5C#N)cc4)nc32)cc1]

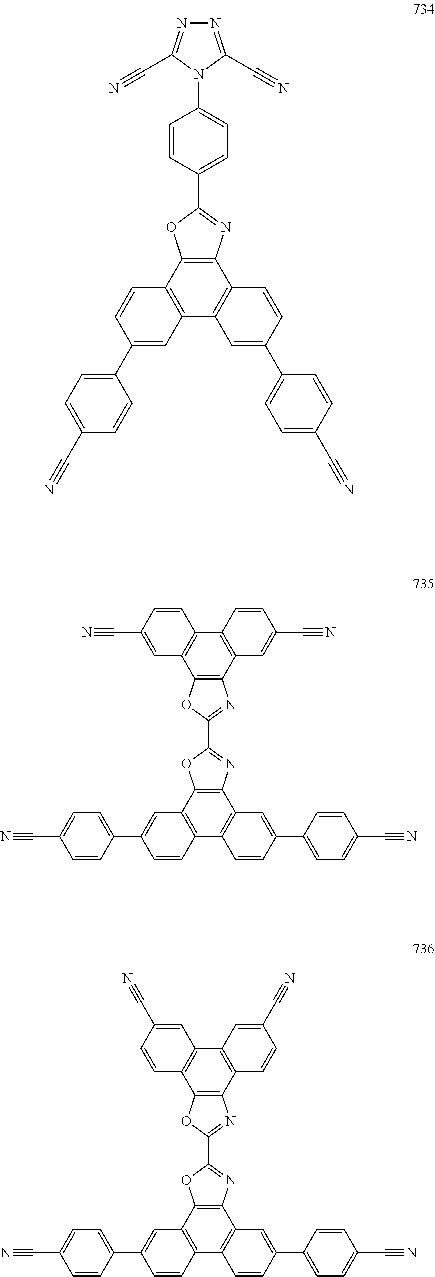 [SMILES: N#Cc1ccc(-c2ccc3c(c2)c2cc(-c4ccc(C#N)cc4)ccc2c2oc(-c4ccc(-n5c(C#N)nnc5C#N)cc4)nc32)cc1 ; N#Cc1ccc(-c2ccc3c4ccc(-c5ccc(C#N)cc5)cc4c4oc(-c5nc6c7cc(C#N)ccc7c7ccc(C#N)cc7c6o5)nc4c3c2)cc1 ; N#Cc1ccc(-c2ccc3c4ccc(-c5ccc(C#N)cc5)cc4c4oc(-c5nc6c7ccc(C#N)cc7c7cc(C#N)ccc7c6o5)nc4c3c2)cc1]

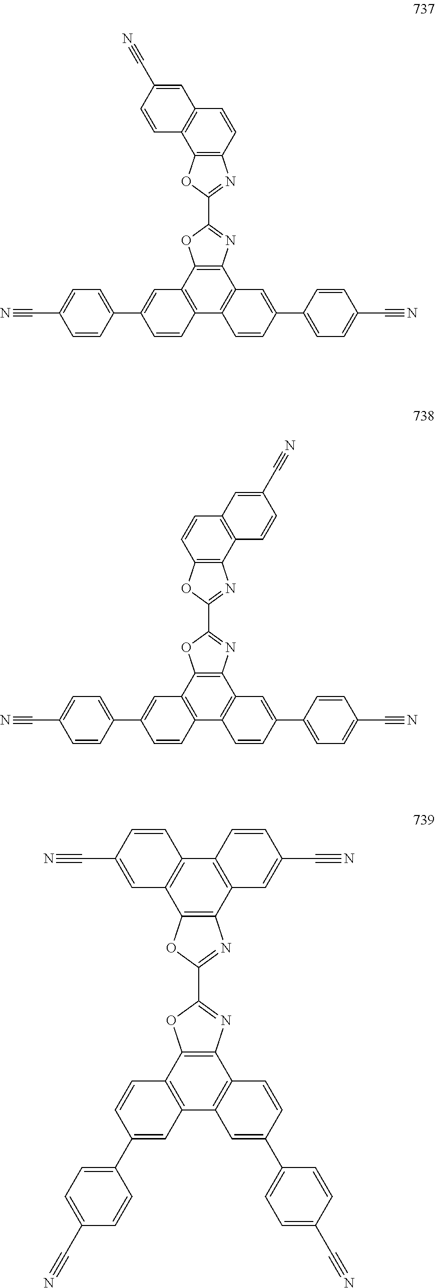 [SMILES: N#Cc1ccc(-c2ccc3c4ccc(-c5ccc(C#N)cc5)cc4c4oc(-c5nc6ccc7cc(C#N)ccc7c6o5)nc4c3c2)cc1 ; N#Cc1ccc(-c2ccc3c4ccc(-c5ccc(C#N)cc5)cc4c4oc(-c5nc6c(ccc7cc(C#N)ccc76)o5)nc4c3c2)cc1 ; N#Cc1ccc(-c2ccc3c(c2)c2cc(-c4ccc(C#N)cc4)ccc2c2oc(-c4nc5c6cc(C#N)ccc6c6ccc(C#N)cc6c5o4)nc32)cc1]

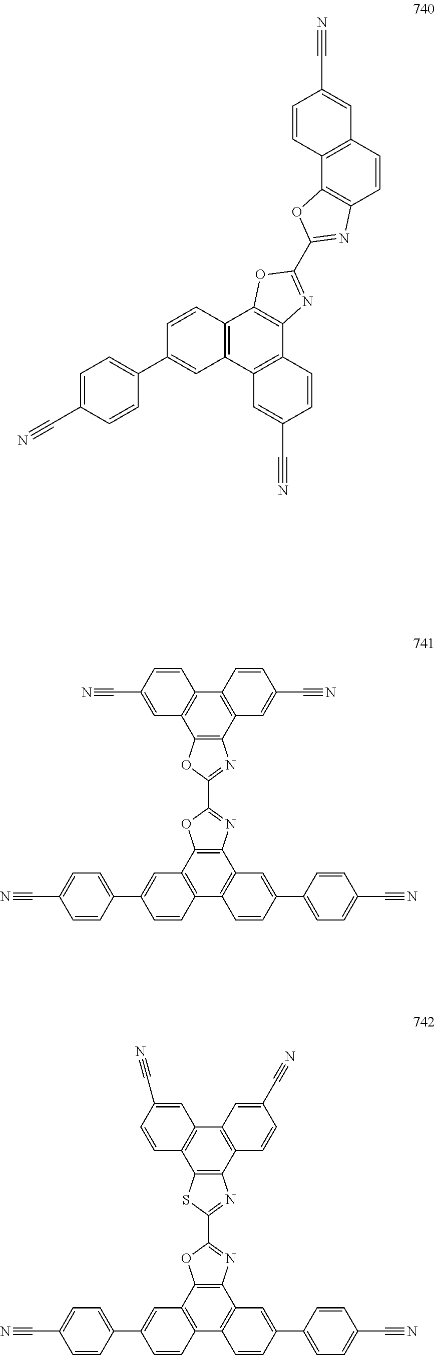 [SMILES: N#Cc1ccc(-c2ccc3c(c2)c2cc(C#N)ccc2c2nc(-c4nc5ccc6cc(C#N)ccc6c5o4)oc32)cc1 ; N#Cc1ccc(-c2ccc3c4ccc(-c5ccc(C#N)cc5)cc4c4oc(-c5nc6c7cc(C#N)ccc7c7ccc(C#N)cc7c6o5)nc4c3c2)cc1 ; N#Cc1ccc(-c2ccc3c4ccc(-c5ccc(C#N)cc5)cc4c4oc(-c5nc6c7ccc(C#N)cc7c7cc(C#N)ccc7c6s5)nc4c3c2)cc1]

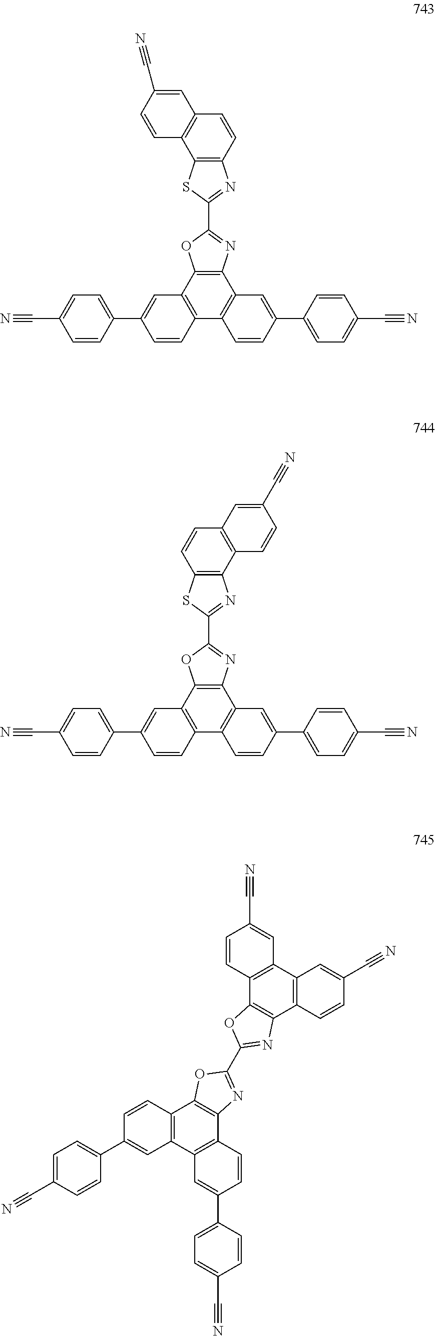 [SMILES: N#Cc1ccc(-c2ccc3c4ccc(-c5ccc(C#N)cc5)cc4c4oc(-c5nc6ccc7cc(C#N)ccc7c6s5)nc4c3c2)cc1 ; N#Cc1ccc(-c2ccc3c4ccc(-c5ccc(C#N)cc5)cc4c4oc(-c5nc6c(ccc7cc(C#N)ccc76)s5)nc4c3c2)cc1 ; N#Cc1ccc(-c2ccc3c(c2)c2cc(-c4ccc(C#N)cc4)ccc2c2oc(-c4nc5c6ccc(C#N)cc6c6cc(C#N)ccc6c5o4)nc32)cc1]

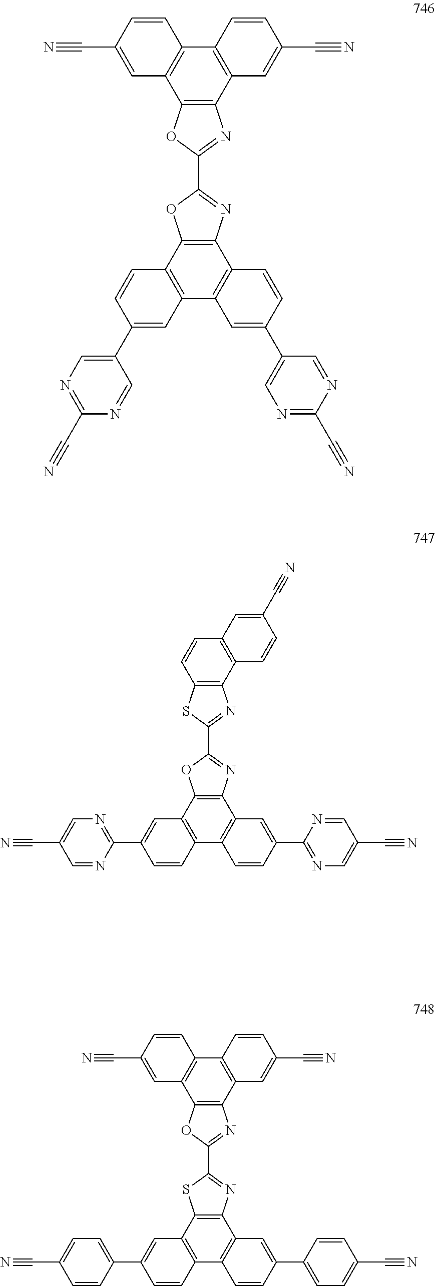 [SMILES: N#Cc1ccc2c3ccc(C#N)cc3c3oc(-c4nc5c6ccc(-c7cnc(C#N)nc7)cc6c6cc(-c7cnc(C#N)nc7)ccc6c5o4)nc3c2c1 ; N#Cc1cnc(-c2ccc3c4ccc(-c5ncc(C#N)cn5)cc4c4oc(-c5nc6c(ccc7cc(C#N)ccc76)s5)nc4c3c2)nc1 ; N#Cc1ccc(-c2ccc3c4ccc(-c5ccc(C#N)cc5)cc4c4sc(-c5nc6c7cc(C#N)ccc7c7ccc(C#N)cc7c6o5)nc4c3c2)cc1]

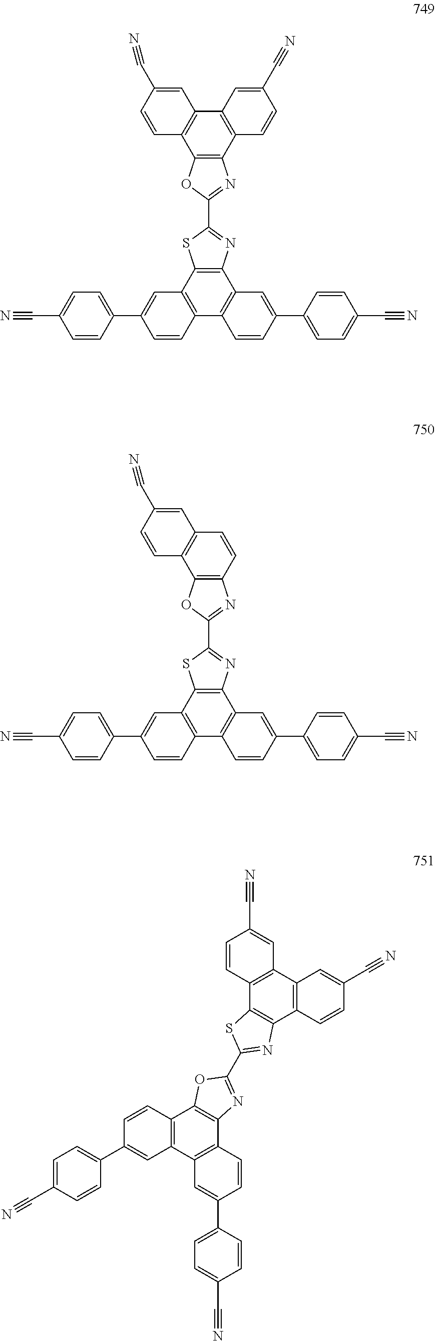 [SMILES: N#Cc1ccc(-c2ccc3c4ccc(-c5ccc(C#N)cc5)cc4c4sc(-c5nc6c7ccc(C#N)cc7c7cc(C#N)ccc7c6o5)nc4c3c2)cc1 ; N#Cc1ccc(-c2ccc3c4ccc(-c5ccc(C#N)cc5)cc4c4sc(-c5nc6ccc7cc(C#N)ccc7c6o5)nc4c3c2)cc1 ; N#Cc1ccc(-c2ccc3c(c2)c2cc(-c4ccc(C#N)cc4)ccc2c2oc(-c4nc5c6ccc(C#N)cc6c6cc(C#N)ccc6c5s4)nc32)cc1]

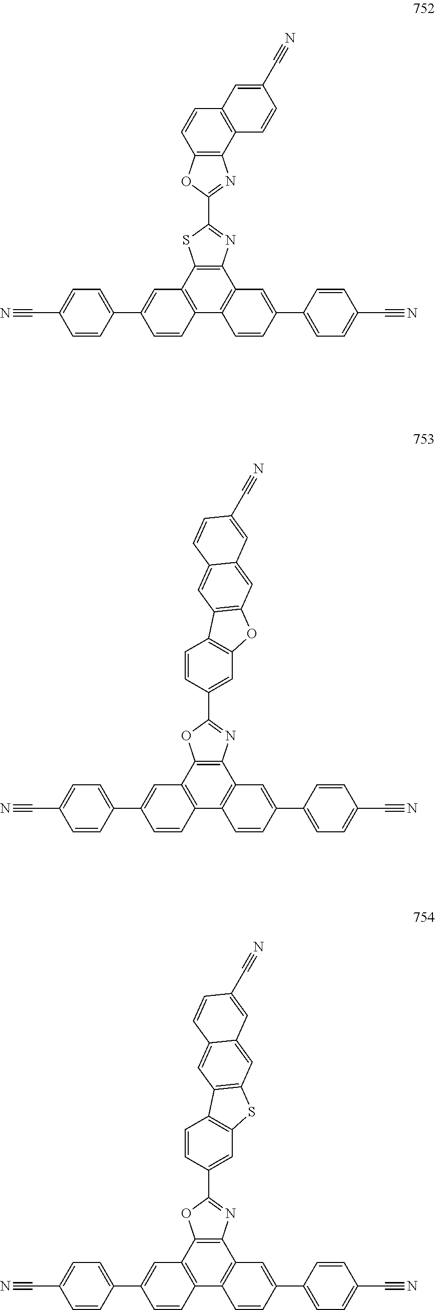 [SMILES: N#Cc1ccc(-c2ccc3c4ccc(-c5ccc(C#N)cc5)cc4c4sc(-c5nc6c(ccc7cc(C#N)ccc76)o5)nc4c3c2)cc1 ; N#Cc1ccc(-c2ccc3c4ccc(-c5ccc(C#N)cc5)cc4c4oc(-c5ccc6c(c5)oc5cc7cc(C#N)ccc7cc56)nc4c3c2)cc1 ; N#Cc1ccc(-c2ccc3c4ccc(-c5ccc(C#N)cc5)cc4c4oc(-c5ccc6c(c5)sc5cc7cc(C#N)ccc7cc56)nc4c3c2)cc1]

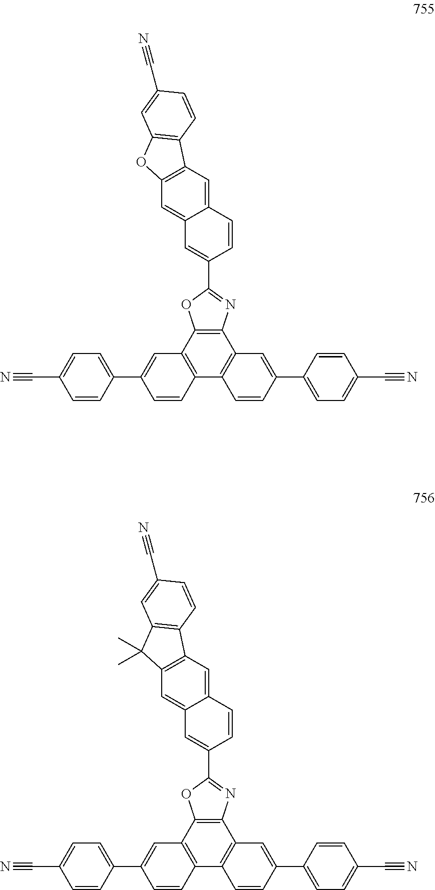 [SMILES: N#Cc1ccc(-c2ccc3c4ccc(-c5ccc(C#N)cc5)cc4c4oc(-c5ccc6cc7c(cc6c5)oc5cc(C#N)ccc57)nc4c3c2)cc1 ; CC1(C)c2cc(C#N)ccc2-c2cc3ccc(-c4nc5c6cc(-c7ccc(C#N)cc7)ccc6c6ccc(-c7ccc(C#N)cc7)cc6c5o4)cc3cc21]

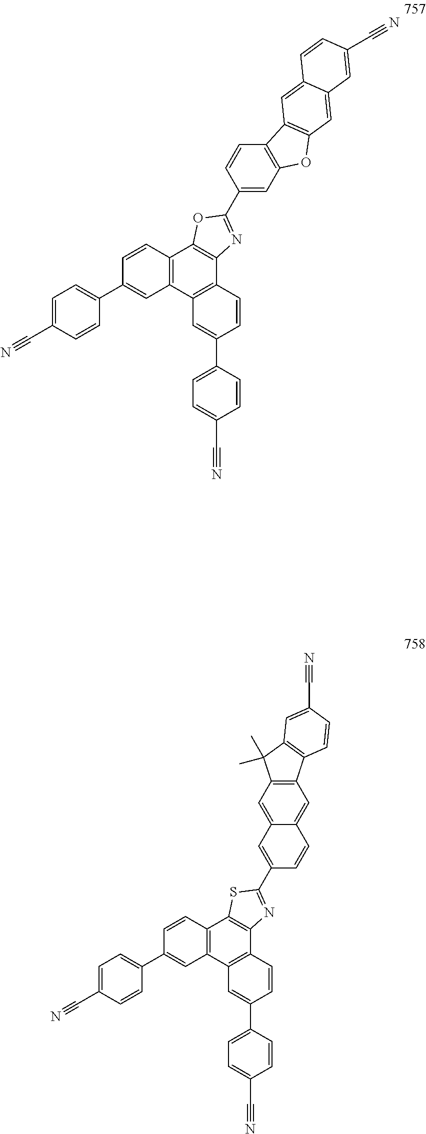 [SMILES: N#Cc1ccc(-c2ccc3c(c2)c2cc(-c4ccc(C#N)cc4)ccc2c2oc(-c4ccc5c(c4)oc4cc6cc(C#N)ccc6cc45)nc32)cc1 ; CC1(C)c2cc(C#N)ccc2-c2cc3ccc(-c4nc5c6ccc(-c7ccc(C#N)cc7)cc6c6cc(-c7ccc(C#N)cc7)ccc6c5s4)cc3cc21]

9. An organic light-emitting device comprising a first electrode, a second electrode, and an organic material layer having one or more layers disposed between the first electrode and the second electrode,
further comprising a light efficiency improving layer (capping layer) formed on at least one side opposite to the organic layer at the top or bottom of the first electrode and the second electrode,
wherein the light efficiency improving layer comprises the compound represented by [Formula I] above.

10. The organic light-emitting device of claim 9, wherein the light efficiency improving layer is formed on at least one of the bottom of the first electrode and the top of the second electrode.
